(19) United States
(12) Patent Application Publication
(10) Pub. No.: US 2024/0341167 A1
(43) Pub. Date: Oct. 10, 2024

(54) ORGANIC ELECTROLUMINESCENT MATERIALS AND DEVICES

(71) Applicant: Universal Display Corporation, Ewing, NJ (US)

(72) Inventors: Tyler FLEETHAM, Yardley, PA (US); Ting-Chih WANG, Lawrenceville, NJ (US); Gregory CENDER, Philadelphia, PA (US)

(73) Assignee: Universal Display Corporation, Ewing, NJ (US)

(21) Appl. No.: 18/602,200

(22) Filed: Mar. 12, 2024

Related U.S. Application Data
(63) Continuation-in-part of application No. 18/491,009, filed on Oct. 20, 2023.
Application No. 18/491,009 is a continuation-in-part of application No. 18/319,182, filed on May 17, 2023.
(60) Provisional application No. 63/506,149, filed on Jun. 5, 2023.
(60) Provisional application No. 63/490,065, filed on Mar. 14, 2023.

(51) Int. Cl. H10K 85/30; C07F 15/00; C09K 11/06; H10K 50/11; H10K 85/40

(52) U.S. Cl. CPC H10K 85/346; C07F 15/0033; C07F 15/0086; C09K 11/06; H10K 85/342; H10K 85/40; C07B 2200/05; C09K 2211/1029; C09K 2211/1033; C09K 2211/1037; C09K 2211/1044; C09K 2211/1048; C09K 2211/1059; C09K 2211/1062; C09K 2211/1074; C09K 2211/185; H10K 50/11

(57) ABSTRACT

An organic light emitting device having an anode; an emissive region; and a cathode is provided. The emissive region can be disposed between the anode and the cathode. The emissive region can include a first compound, D1, which is a first metal complex; and a second compound, D2, which is a second metal complex, where D1 and D2 are different. The emission from the OLED does not primarily originate from D2 or from an exciplex between D1 and D2, the emission from the OLED has an emission peak maximum between 300 to 2000 nm; and, when the emission from the OLED has a peak between 550 and 700 nm, then D2 is not a Be, Al, or Ir complex. Consumer products containing the OLED are also provided.

### CROSS-REFERENCE TO RELATED APPLICATIONS

This application is a continuation-in-part of co-pending U.S. patent application Ser. No. 18/491,009, filed on Oct. 20, 2023, which is a continuation-in-part of U.S. patent application Ser. No. 18/319,182, filed on May 17, 2023. This application also claims priority under 35 U.S.C. § 119(e) to U.S. Provisional Application No. 63/506,149, filed on Jun. 5, 2023, and 63/490,065, filed on Mar. 14, 2023, the entire contents of all the above referenced applications are incorporated herein by reference.

### FIELD

The present invention relates to devices and techniques for fabricating organic emissive devices, such as organic light emitting diodes, and devices and techniques including the same.

### BACKGROUND

Opto-electronic devices that make use of organic materials are becoming increasingly desirable for a number of reasons. Many of the materials used to make such devices are relatively inexpensive, so organic opto-electronic devices have the potential for cost advantages over inorganic devices. In addition, the inherent properties of organic materials, such as their flexibility, may make them well suited for particular applications such as fabrication on a flexible substrate. Examples of organic opto-electronic devices include organic light emitting diodes/devices (OLEDs), organic phototransistors, organic scintillators, organic photovoltaic cells, and organic photodetectors. For OLEDs, the organic materials may have performance advantages over conventional materials.

OLEDs make use of thin organic films that emit light when voltage is applied across the device. OLEDs are becoming an increasingly interesting technology for use in applications such as displays, illumination, and backlighting.

One application for emissive molecules is a full color display. Industry standards for such a display call for pixels adapted to emit particular colors, referred to as “saturated” colors. In particular, these standards call for saturated red, green, and blue pixels. Alternatively, the OLED can be designed to emit white light. In conventional liquid crystal displays emission from a white backlight is filtered using absorption filters to produce red, green and blue emission. The same technique can also be used with OLEDs. The white OLED can be either a single emissive layer (EML) device or a stack structure. Color may be measured using CIE coordinates, which are well known to the art.

### SUMMARY

According to an embodiment, an organic light emitting diode/device (OLED) is also provided. The OLED can include an anode, a cathode, and an organic layer, disposed between the anode and the cathode. According to an embodiment, the organic light emitting device is incorporated into one or more device selected from a consumer product, an electronic component module, and/or a lighting panel.

### BRIEF DESCRIPTION OF THE DRAWINGS

FIG. 1 shows an organic light emitting device.

FIG. 2 shows an inverted organic light emitting device that does not have a separate electron transport layer.

### DETAILED DESCRIPTION

### A. Terminology

Unless otherwise specified, the below terms used herein are defined as follows:

As used herein, “top” means furthest away from the substrate, while “bottom” means closest to the substrate. Where a first layer is described as “disposed over” a second layer, the first layer is disposed further away from substrate. There may be other layers between the first and second layer, unless it is specified that the first layer is “in contact with” the second layer. For example, a cathode may be described as “disposed over” an anode, even though there are various organic layers in between.

As used herein, “solution processable” means capable of being dissolved, dispersed, or transported in and/or deposited from a liquid medium, either in solution or suspension form.

As used herein, and as would be generally understood by one skilled in the art, a first “Highest Occupied Molecular Orbital” (HOMO) or “Lowest Unoccupied Molecular Orbital” (LUMO) energy level is “greater than” or “higher than” a second HOMO or LUMO energy level if the first energy level is closer to the vacuum energy level. Since ionization potentials (IP) are measured as a negative energy relative to a vacuum level, a higher HOMO energy level corresponds to an IP having a smaller absolute value (an IP that is less negative). Similarly, a higher LUMO energy level corresponds to an electron affinity (EA) having a smaller absolute value (an EA that is less negative). On a conventional energy level diagram, with the vacuum level at the top, the LUMO energy level of a material is higher than the HOMO energy level of the same material. A “higher” HOMO or LUMO energy level appears closer to the top of such a diagram than a “lower” HOMO or LUMO energy level.

As used herein, and as would be generally understood by one skilled in the art, a first work function is “greater than” or “higher than” a second work function if the first work function has a higher absolute value. Because work functions are generally measured as negative numbers relative to vacuum level, this means that a “higher” work function is more negative. On a conventional energy level diagram, with the vacuum level at the top, a “higher” work function is illustrated as further away from the vacuum level in the downward direction. Thus, the definitions of HOMO and LUMO energy levels follow a different convention than work functions.

The terms “halo,” “halogen,” and “halide” are used interchangeably and refer to fluorine, chlorine, bromine, and iodine.

The term “acyl” refers to a substituted carbonyl group (—C(O)—Rₛ).

The term “ester” refers to a substituted oxycarbonyl (—O—C(O)—Rₛ or —C(O)—O—Rₛ) group.

The term “ether” refers to an —ORₛ group.

The terms “sulfanyl” or “thio-ether” are used interchangeably and refer to a —SRₛ group.

The term “selenyl” refers to a —SeRₛ group.

The term “sulfinyl” refers to a —S(O)—Rₛ group.

The term “sulfonyl” refers to a —SO₂—Rₛ group.

The term “phosphino” refers to a group containing at least one phosphorus atom bonded to the relevant structure. Common examples of phosphino groups include, but are not limited to, groups such as a —P(Rₛ)₂ group or a —PO(Rₛ)₂ group, wherein each Rₛ can be same or different.

The term “silyl” refers to a group containing at least one silicon atom bonded to the relevant structure. Common examples of silyl groups include, but are not limited to, groups such as a —Si(Rₛ)₃ group, wherein each Rₛ can be same or different.

The term “germyl” refers to a group containing at least one germanium atom bonded to the relevant structure. Common examples of germyl groups include, but are not limited to, groups such as a —Ge(Rₛ)₃ group, wherein each Rₛ can be same or different.

The term “boryl” refers to a group containing at least one boron atom bonded to the relevant structure. Common examples of boryl groups include, but are not limited to, groups such as a —B(Rₛ)₂ group or its Lewis adduct —B(Rₛ)₃ group, wherein Rₛ can be same or different.

In each of the above, Rₛ can be hydrogen or a substituent selected from the group consisting of the general substituents as defined in this application. Preferred Rₛ is selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, and combination thereof. More preferably Rₛ is selected from the group consisting of alkyl, cycloalkyl, aryl, heteroaryl, and combination thereof.

The term “alkyl” refers to and includes both straight and branched chain alkyl groups having an alkyl carbon atom bonded to the relevant structure. Preferred alkyl groups are those containing from one to fifteen carbon atoms, preferably one to nine carbon atoms, and includes methyl, ethyl, propyl, 1-methylethyl, butyl, 1-methylpropyl, 2-methylpropyl, pentyl, 1-methylbutyl, 2-methylbutyl, 3-methylbutyl, 1,1-dimethylpropyl, 1,2-dimethylpropyl, 2,2-dimethylpropyl, and the like. Additionally, the alkyl group can be further substituted.

The term “cycloalkyl” refers to and includes monocyclic, polycyclic, and spiro alkyl groups having a ring alkyl carbon atom bonded to the relevant structure. Preferred cycloalkyl groups are those containing 3 to 12 ring carbon atoms and includes cyclopropyl, cyclopentyl, cyclohexyl, bicyclo[3.1.1]heptyl, spiro[4.5]decyl, spiro[5.5]undecyl, adamantyl, and the like. Additionally, the cycloalkyl group can be further substituted.

The terms “heteroalkyl” or “heterocycloalkyl” refer to an alkyl or a cycloalkyl group, respectively, having at least one carbon atom replaced by a heteroatom. Optionally the at least one heteroatom is selected from O, S, N, P, B, Si, Ge and Se, preferably, O, S or N. Additionally, the heteroalkyl or heterocycloalkyl group can be further substituted.

The term “alkenyl” refers to and includes both straight and branched chain alkene groups. Alkenyl groups are essentially alkyl groups that include at least one carbon-carbon double bond in the alkyl chain with one carbon atom from the carbon-carbon double bond that is bonded to the relevant structure. Cycloalkenyl groups are essentially cycloalkyl groups that include at least one carbon-carbon double bond in the cycloalkyl ring. The term “heteroalkenyl” as used herein refers to an alkenyl group having at least one carbon atom replaced by a heteroatom. Optionally the at least one heteroatom is selected from O, S, N, P, B, Si, Ge, and Se, preferably, O, S, or N. Preferred alkenyl, cycloalkenyl, or heteroalkenyl groups are those containing two to fifteen carbon atoms. Additionally, the alkenyl, cycloalkenyl, or heteroalkenyl group can be further substituted.

The term “alkynyl” refers to and includes both straight and branched chain alkyne groups. Alkynyl groups are essentially alkyl groups that include at least one carbon-carbon triple bond in the alkyl chain with one carbon atom from the carbon-carbon triple bond that is bonded to the relevant structure. Preferred alkynyl groups are those containing two to fifteen carbon atoms. Additionally, the alkynyl group can be further substituted.

The terms “aralkyl” or “arylalkyl” are used interchangeably and refer to an aryl-substituted alkyl group having an alkyl carbon atom bonded to the relevant structure. Additionally, the aralkyl group can be further substituted.

The term “heterocyclic group” refers to and includes aromatic and non-aromatic cyclic groups containing at least one heteroatom. Optionally the at least one heteroatom is selected from O, S, Se, N, P, B, Si, Ge, and Se, preferably, O, S, N, or B. Hetero-aromatic cyclic groups may be used interchangeably with heteroaryl. Preferred hetero-non-aromatic cyclic groups are those containing 3 to 10 ring atoms, preferably those containing 3 to 7 ring atoms, which includes at least one hetero atom, and includes cyclic amines such as morpholino, piperidino, pyrrolidino, and the like, and cyclic ethers/thio-ethers, such as tetrahydrofuran, tetrahydropyran, tetrahydrothiophene, and the like. Additionally, the heterocyclic group can be further substituted or fused.

The term “aryl” refers to and includes both single-ring and polycyclic aromatic hydrocarbyl groups. The polycyclic rings may have two or more rings in which two carbons are common to two adjoining rings (the rings are “fused”). Preferred aryl groups are those containing six to thirty carbon atoms, preferably six to twenty-four carbon atoms, six to eighteen carbon atoms, and more preferably six to twelve carbon atoms. Especially preferred is an aryl group having six carbons, ten carbons, twelve carbons, fourteen carbons, or eighteen carbons. Suitable aryl groups include phenyl, biphenyl, triphenyl, triphenylene, tetraphenylene, naphthalene, anthracene, phenalene, phenanthrene, pyrene, chrysene, perylene, and azulene, preferably phenyl, biphenyl, triphenyl, triphenylene, and naphthalene. Additionally, the aryl group can be further substituted or fused, such as, without limitation, fluorene.

The term “heteroaryl” refers to and includes both single-ring aromatic groups and polycyclic aromatic ring systems that include at least one heteroatom. The heteroatoms include, but are not limited to O, S, Se, N, P, B, Si, Ge, and Se. In many instances, O, S, N, or B are the preferred heteroatoms. Hetero-single ring aromatic systems are preferably single rings with 5 or 6 ring atoms, and the ring can have from one to six heteroatoms. The hetero-polycyclic ring systems can have two or more aromatic rings in which two atoms are common to two adjoining rings (the rings are “fused”) wherein at least one of the rings is a heteroaryl. The hetero-polycyclic aromatic ring systems can have from one to six heteroatoms per ring of the polycyclic aromatic ring system. Preferred heteroaryl groups are those containing three to thirty carbon atoms, preferably three to twenty-four carbon atoms, three to eighteen carbon atoms, and more preferably three to twelve carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, selenophenodipyridine, azaborine, borazine, 5λ²,9λ²-diaza-13b-boranaphtho[2,3,4-de]anthracene, 5λ²-benzo[d]benzo[4,5]imidazo[3,2-a]imidazole, and 5,9-dioxa-13b-boranaphtho[3,2,1-de]anthracene; preferably dibenzothiophene, dibenzofuran, dibenzoselenophene, carbazole, indolocarbazole, imidazole, pyridine, triazine, benzimidazole, 5λ²,9λ²-diaza-13b-boranaphtho[2,3,4-de]anthracene, 5λ²-benzo[d]benzo[4,5]imidazo[3,2-a]imidazole, and 5,9-dioxa-13b-boranaphtho[3,2,1-de]anthracene. Additionally, the heteroaryl group can be further substituted or fused.

Of the aryl and heteroaryl groups listed above, the groups of triphenylene, naphthalene, anthracene, dibenzothiophene, dibenzofuran, dibenzoselenophene, carbazole, indolocarbazole, imidazole, pyridine, pyrazine, pyrimidine, triazine, benzimidazole, 5λ²,9λ²-diaza-13b-boranaphtho[2,3,4-de]anthracene, 5λ²-benzo[d]benzo[4,5]imidazo[3,2-a]imidazole, 5,9-dioxa-13b-boranaphtho[3,2,1-de]anthracene, and the respective aza-analogs of each thereof are of particular interest.

In many instances, the General Substituents are selected from the group consisting of deuterium, halogen, alkyl, cycloalkyl, heteroalkyl, heterocycloalkyl, arylalkyl, alkoxy, aryloxy, amino, silyl, germyl, boryl, alkenyl, cycloalkenyl, heteroalkenyl, alkynyl, aryl, heteroaryl, acyl, carboxylic acid, ether, ester, nitrile, isonitrile, sulfanyl, selenyl, sulfinyl, sulfonyl, phosphino, and combinations thereof.

In some instances, the Preferred General Substituents are selected from the group consisting of deuterium, fluorine, alkyl, cycloalkyl, heteroalkyl, alkoxy, aryloxy, amino, silyl, germyl, boryl, alkenyl, cycloalkenyl, heteroalkenyl, aryl, heteroaryl, nitrile, isonitrile, sulfanyl, and combinations thereof.

In some instances, the More Preferred General Substituents are selected from the group consisting of deuterium, fluorine, alkyl, cycloalkyl, alkoxy, aryloxy, amino, silyl, germyl, boryl, aryl, heteroaryl, nitrile, sulfanyl, and combinations thereof.

In some instances, the Even More Preferred General Substituents are selected from the group consisting of deuterium, fluorine, alkyl, cycloalkyl, silyl, aryl, heteroaryl, nitrile, and combinations thereof.

In yet other instances, the Most Preferred General Substituents are selected from the group consisting of deuterium, alkyl, cycloalkyl, aryl, heteroaryl, and combinations thereof.

The terms “substituted” and “substitution” refer to a substituent other than H that is bonded to the relevant position, e.g., a carbon or nitrogen. For example, when R¹ represents mono-substitution, then one R must be other than H (i.e., a substitution). Similarly, when R¹ represents di-substitution, then two of R¹ must be other than H. Similarly, when R¹ represents zero or no substitution, R¹, for example, can be a hydrogen for all available valencies of ring atoms, as in carbon atoms for benzene and the nitrogen atom in pyrrole, or simply represents nothing for ring atoms with fully filled valencies, e.g., the nitrogen atom in pyridine. The maximum number of substitutions possible in a ring structure will depend on the total number of available valencies in the ring atoms.

As used herein, “combinations thereof” indicates that one or more members of the applicable list are combined to form a known or chemically stable arrangement that one of ordinary skill in the art can envision from the applicable list. For example, an alkyl and deuterium can be combined to form a partial or fully deuterated alkyl group; a halogen and alkyl can be combined to form a halogenated alkyl substituent; and a halogen, alkyl, and aryl can be combined to form a halogenated arylalkyl. In one instance, the term substitution includes a combination of two to four of the listed groups. In another instance, the term substitution includes a combination of two to three groups. In yet another instance, the term substitution includes a combination of two groups. Preferred combinations of substituent groups are those that contain up to fifty atoms that are not hydrogen or deuterium, or those which include up to forty atoms that are not hydrogen or deuterium, or those that include up to thirty atoms that are not hydrogen or deuterium. In many instances, a preferred combination of substituent groups will include up to twenty atoms that are not hydrogen or deuterium.

The “aza” designation in the fragments described herein, i.e. aza-dibenzofuran, aza-dibenzothiophene, etc. means that one or more of the C—H groups in the respective aromatic ring can be replaced by a nitrogen atom, for example, and without any limitation, azatriphenylene encompasses both dibenzo[f;h]quinoxaline and dibenzo[f;h]quinoline. One of ordinary skill in the art can readily envision other nitrogen analogs of the aza-derivatives described above, and all such analogs are intended to be encompassed by the terms as set forth herein.

As used herein, “deuterium” refers to an isotope of hydrogen. Deuterated compounds can be readily prepared using methods known in the art. For example, U.S. Pat. No. 8,557,400, Patent Pub. No. WO 2006/095951, and U.S. Pat. Application Pub. No. US 2011/0037057, which are hereby incorporated by reference in their entireties, describe the making of deuterium-substituted organometallic complexes. Further reference is made to Ming Yan, et al., Tetrahedron 2015, 71, 1425-30 and Atzrodt et al., Angew. Chem. Int. Ed. (Reviews) 2007, 46, 7744-65, which are incorporated by reference in their entireties, describe the deuteration of the methylene hydrogens in benzyl amines and efficient pathways to replace aromatic ring hydrogens with deuterium, respectively.

As used herein, any specifically listed substituent, such as, without limitation, methyl, phenyl, pyridyl, etc. includes undeuterated, partially deuterated, and fully deuterated versions thereof. Similarly, classes of substituents such as, without limitation, alkyl, aryl, cycloalkyl, heteroaryl, etc. also include undeuterated, partially deuterated, and fully deuterated versions thereof. Unless otherwise specified, atoms in chemical structures without valences fully filled by H or D should be considered to include undeuterated, partially deuterated, and fully deuterated versions thereof. For example, the chemical structure of

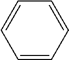

implies to include C₆H₆, C₆D₆, C₆H₃D₃, and any other partially deuterated variants thereof. Some common basic partially or fully deuterated group include, without limitation, CD₃, CD₂C(CH₃)₃, C(CD₃)₃, and C₆D₅.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or attached fragment are considered to be equivalent.

In some instances, a pair of substituents in the molecule can be optionally joined or fused into a ring. The preferred ring is a five to nine-membered carbocyclic or heterocyclic ring, includes both instances where the portion of the ring formed by the pair of substituents is saturated and where the portion of the ring formed by the pair of substituents is unsaturated. In yet other instances, a pair of adjacent substituents can be optionally joined or fused into a ring. As used herein, “adjacent” means that the two substituents involved can be on the same ring next to each other, or on two neighboring rings having the two closest available substitutable positions, such as 2, 2′ positions in a biphenyl, or 1, 8 position in a naphthalene.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

Layers, materials, regions, and devices may be described herein in reference to the color of light they emit. In general, as used herein, an emissive region that is described as producing a specific color of light may include one or more emissive layers disposed over each other in a stack.

As used herein, a “red” layer, material, region, or device refers to one that emits light in the range of about 580-700 nm or having a highest peak in its emission spectrum in that region. Similarly, a “green” layer, material, region, or device refers to one that emits or has an emission spectrum with a peak wavelength in the range of about 500-600 nm; a “blue” layer, material, or device refers to one that emits or has an emission spectrum with a peak wavelength in the range of about 400-500 nm; and a “yellow” layer, material, region, or device refers to one that has an emission spectrum with a peak wavelength in the range of about 540-600 nm. In some arrangements, separate regions, layers, materials, regions, or devices may provide separate “deep blue” and a “light blue” light. As used herein, in arrangements that provide separate “light blue” and “deep blue”, the “deep blue” component refers to one having a peak emission wavelength that is at least about 4 nm less than the peak emission wavelength of the “light blue” component. Typically, a “light blue” component has a peak emission wavelength in the range of about 465-S00 nm, and a “deep blue” component has a peak emission wavelength in the range of about 400-470 nm, though these ranges may vary for some configurations. Similarly, a color altering layer refers to a layer that converts or modifies another color of light to light having a wavelength as specified for that color. For example, a “red” color filter refers to a filter that results in light having a wavelength in the range of about 580-700 nm. In general, there are two classes of color altering layers: color filters that modify a spectrum by removing unwanted wavelengths of light, and color changing layers that convert photons of higher energy to lower energy. A component “of a color” refers to a component that, when activated or used, produces or otherwise emits light having a particular color as previously described. For example, a “first emissive region of a first color” and a “second emissive region of a second color different than the first color” describes two emissive regions that, when activated within a device, emit two different colors as previously described.

As used herein, emissive materials, layers, and regions may be distinguished from one another and from other structures based upon light initially generated by the material, layer or region, as opposed to light eventually emitted by the same or a different structure. The initial light generation typically is the result of an energy level change resulting in emission of a photon. For example, an organic emissive material may initially generate blue light, which may be converted by a color filter, quantum dot or other structure to red or green light, such that a complete emissive stack or sub-pixel emits the red or green light. In this case the initial emissive material or layer may be referred to as a “blue” component, even though the sub-pixel is a “red” or “green” component.

In some cases, it may be preferable to describe the color of a component such as an emissive region, sub-pixel, color altering layer, or the like, in terms of 1931 CIE coordinates. For example, a yellow emissive material may have multiple peak emission wavelengths, one in or near an edge of the “green” region, and one within or near an edge of the “red” region as previously described. Accordingly, as used herein, each color term also corresponds to a shape in the 1931 CIE coordinate color space. The shape in 1931 CIE color space is constructed by following the locus between two color points and any additional interior points. For example, interior shape parameters for red, green, blue, and yellow may be defined as shown below:

| Color | CIE Shape Parameters |
|---|---|
| Central Red | Locus: [0.6270, 0.3725]; [0.7347, 0.2653]; |
| | Interior: [0.5086, 0.2657] |
| Central Green | Locus: [0.0326, 0.3530]; [0.3731, 0.6245]; |
| | Interior: [0.2268, 0.3321 |
| Central Blue | Locus: [0.1746, 0.0052]; [0.0326, 0.3530]; |
| | Interior: [0.2268, 0.3321] |
| Central Yellow | Locus: [0.3731, 0.6245]; [0.6270, 0.3725]; |
| | Interior: [0.3700, 0.4087]; [0.2886, 0.4572] |

More details on OLEDs, and the definitions described above, can be found in U.S. Pat. No. 7,279,704, which is incorporated herein by reference in its entirety.

Generally, an OLED comprises at least one organic layer disposed between and electrically connected to an anode and a cathode. When a current is applied, the anode injects holes and the cathode injects electrons into the organic layer(s). The injected holes and electrons each migrate toward the oppositely charged electrode. When an electron and hole localize on the same molecule, an “exciton,” which is a localized electron-hole pair having an excited energy state, is formed. Light is emitted when the exciton relaxes via a photoemissive mechanism. In some cases, the exciton may be localized on an excimer or an exciplex. Non-radiative mechanisms, such as thermal relaxation, may also occur, but are generally considered undesirable.

The initial OLEDs used emissive molecules that emitted light from their singlet states (“fluorescence”) as disclosed, for example, in U.S. Pat. No. 4,769,292, which is incorporated by reference in its entirety. Fluorescent emission generally occurs in a time frame of less than 10 nanoseconds.

More recently, OLEDs having emissive materials that emit light from triplet states (“phosphorescence”) have been demonstrated. Baldo et al., “Highly Efficient Phosphorescent Emission from Organic Electroluminescent Devices,” Nature, vol. 395, 151-154, 1998; (“Baldo-I”) and Baldo et al., “Very high-efficiency green organic light-emitting devices based on electrophosphorescence,” Appl. Phys. Lett., vol. 75, No. 3, 4-6 (1999) (“Baldo-II”), are incorporated by reference in their entireties. Phosphorescence is described in more detail in U.S. Pat. No. 7,279,704 at cols. 5-6, which are incorporated by reference.

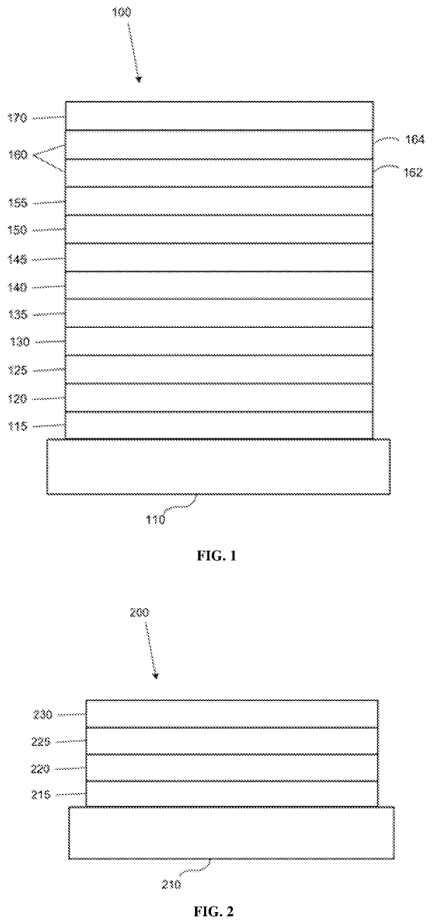

FIG. 1 shows an organic light emitting device 100. The figures are not necessarily drawn to scale. Device 100 may include a substrate 110, an anode 115, a hole injection layer 120, a hole transport layer 125, an electron blocking layer 130, an emissive layer 135, a hole blocking layer 140, an electron transport layer 145, an electron injection layer 150, a protective layer 155, a cathode 160, and a barrier layer 170. Cathode 160 is a compound cathode having a first conductive layer 162 and a second conductive layer 164. Device 100 may be fabricated by depositing the layers described, in order. The properties and functions of these various layers, as well as example materials, are described in more detail in U.S. Pat. No. 7,279,704 at cols. 6-10, which are incorporated by reference.

More examples for each of these layers are available. For example, a flexible and transparent substrate-anode combination is disclosed in U.S. Pat. No. 5,844,363, which is incorporated by reference in its entirety. An example of a p-doped hole transport layer is m-MTDATA doped with F₄-TCNQ at a molar ratio of 50:1, as disclosed in U.S. Patent Application Publication No. 2003/0230980, which is incorporated by reference in its entirety. Examples of emissive and host materials are disclosed in U.S. Pat. No. 6,303,238 to Thompson et al., which is incorporated by reference in its entirety. An example of an n-doped electron transport layer is BPhen doped with Li at a molar ratio of 1:1, as disclosed in U.S. Patent Application Publication No. 2003/0230980, which is incorporated by reference in its entirety. U.S. Pat. Nos. 5,703,436 and 5,707,745, which are incorporated by reference in their entireties, disclose examples of cathodes including compound cathodes having a thin layer of metal such as Mg:Ag with an overlying transparent, electrically-conductive, sputter-deposited ITO layer. The theory and use of blocking layers is described in more detail in U.S. Pat. No. 6,097,147 and U.S. Patent Application Publication No. 2003/0230980, which are incorporated by reference in their entireties. Examples of injection layers are provided in U.S. Patent Application Publication No. 2004/0174116, which is incorporated by reference in its entirety. Barrier layer 170 may be a single- or multi-layer barrier and may cover or surround the other layers of the device. The barrier layer 170 may also surround the substrate 110, and/or it may be arranged between the substrate and the other layers of the device. The barrier also may be referred to as an encapsulant, encapsulation layer, protective layer, or permeation barrier, and typically provides protection against permeation by moisture, ambient air, and other similar materials through to the other layers of the device. Examples of barrier layer materials and structures are provided in U.S. Pat. Nos. 6,537,688, 6,597,111, 6,664,137, 6,835,950, 6,888,305, 6,888,307, 6,897,474, 7,187,119, and 7,683,534, each of which is incorporated by reference in its entirety.

FIG. 2 shows an inverted OLED 200. The device includes a substrate 210, a cathode 215, an emissive layer 220, a hole transport layer 225, and an anode 230. Device 200 may be fabricated by depositing the layers described, in order. Because the most common OLED configuration has a cathode disposed over the anode, and device 200 has cathode 215 disposed under anode 230, device 200 may be referred to as an “inverted” OLED. Materials similar to those described with respect to device 100 may be used in the corresponding layers of device 200. FIG. 2 provides one example of how some layers may be omitted from the structure of device 100.

The simple layered structure illustrated in FIGS. 1 and 2 is provided by way of non-limiting example, and it is understood that embodiments of the invention may be used in connection with a wide variety of other structures. The specific materials and structures described are exemplary in nature, and other materials and structures may be used. Functional OLEDs may be achieved by combining the various layers described in different ways, or layers may be omitted entirely, based on design, performance, and cost factors. Other layers not specifically described may also be included. Materials other than those specifically described may be used. Although many of the examples provided herein describe various layers as comprising a single material, it is understood that combinations of materials, such as a mixture of host and dopant, or more generally a mixture, may be used. Also, the layers may have various sublayers. The names given to the various layers herein are not intended to be strictly limiting. For example, in device 200, hole transport layer 225 transports holes and injects holes into emissive layer 220, and may be described as a hole transport layer or a hole injection layer. In one embodiment, an OLED may be described as having an “organic layer” disposed between a cathode and an anode. This organic layer may comprise a single layer, or may further comprise multiple layers of different organic materials as described, for example, with respect to FIGS. 1 and 2.

Structures and materials not specifically described may also be used, such as OLEDs comprised of polymeric materials (PLEDs) such as disclosed in U.S. Pat. No. 5,247,190 to Friend et al., which is incorporated by reference in its entirety. By way of further example, OLEDs having a single organic layer may be used. OLEDs may be stacked, for example as described in U.S. Pat. No. 5,707,745 to Forrest et al, which is incorporated by reference in its entirety. The OLED structure may deviate from the simple layered structure illustrated in FIGS. 1 and 2. For example, the substrate may include an angled reflective surface to improve out-coupling, such as a mesa structure as described in U.S. Pat. No. 6,091,195 to Forrest et al., and/or a pit structure as described in U.S. Pat. No. 5,834,893 to Bulovic et al., which are incorporated by reference in their entireties.

In some embodiments disclosed herein, emissive layers or materials, such as emissive layer 135 and emissive layer 220 shown in FIGS. 1-2, respectively, may include quantum dots. An “emissive layer” or “emissive material” as disclosed herein may include an organic emissive material and/or an emissive material that contains quantum dots or equivalent structures, unless indicated to the contrary explicitly or by context according to the understanding of one of skill in the art. In general, an emissive layer includes emissive material within a host matrix. Such an emissive layer may include only a quantum dot material which converts light emitted by a separate emissive material or other emitter, or it may also include the separate emissive material or other emitter, or it may emit light itself directly from the application of an electric current. Similarly, a color altering layer, color filter, upconversion, or downconversion layer or structure may include a material containing quantum dots, though such layer may not be considered an “emissive layer” as disclosed herein. In general, an “emissive layer” or material is one that emits an initial light based on an injected electrical charge, where the initial light may be altered by another layer such as a color filter or other color altering layer that does not itself emit an initial light within the device, but may re-emit altered light of a different spectra content based upon absorption of the initial light emitted by the emissive layer and downconversion to a lower energy light emission. In some embodiments disclosed herein, the color altering layer, color filter, upconversion, and/or downconversion layer may be disposed outside of an OLED device, such as above or below an electrode of the OLED device.

Unless otherwise specified, any of the layers of the various embodiments may be deposited by any suitable method. For the organic layers, preferred methods include thermal evaporation, ink-jet, such as described in U.S. Pat. Nos. 6,013,982 and 6,087,196, which are incorporated by reference in their entireties, organic vapor phase deposition (OVPD), such as described in U.S. Pat. No. 6,337,102 to Forrest et al., which is incorporated by reference in its entirety, and deposition by organic vapor jet printing (OVJP), such as described in U.S. Pat. No. 7,431,968, which is incorporated by reference in its entirety. Other suitable deposition methods include spin coating and other solution based processes. Solution based processes are preferably carried out in nitrogen or an inert atmosphere. For the other layers, preferred methods include thermal evaporation. Preferred patterning methods include deposition through a mask, cold welding such as described in U.S. Pat. Nos. 6,294,398 and 6,468,819, which are incorporated by reference in their entireties, and patterning associated with some of the deposition methods such as ink-jet and OVJD. Other methods may also be used. The materials to be deposited may be modified to make them compatible with a particular deposition method. For example, substituents such as alkyl and aryl groups, branched or unbranched, and preferably containing at least 3 carbons, may be used in small molecules to enhance their ability to undergo solution processing. Substituents having 20 carbons or more may be used, and 3-20 carbons is a preferred range. Materials with asymmetric structures may have better solution processibility than those having symmetric structures, because asymmetric materials may have a lower tendency to recrystallize. Dendrimer substituents may be used to enhance the ability of small molecules to undergo solution processing.

Devices fabricated in accordance with embodiments of the present invention may further optionally comprise a barrier layer. One purpose of the barrier layer is to protect the electrodes and organic layers from damaging exposure to harmful species in the environment including moisture, vapor and/or gases, etc. The barrier layer may be deposited over, under or next to a substrate, an electrode, or over any other parts of a device including an edge. The barrier layer may comprise a single layer, or multiple layers. The barrier layer may be formed by various known chemical vapor deposition techniques and may include compositions having a single phase as well as compositions having multiple phases. Any suitable material or combination of materials may be used for the barrier layer. The barrier layer may incorporate an inorganic or an organic compound or both. The preferred barrier layer comprises a mixture of a polymeric material and a non-polymeric material as described in U.S. Pat. No. 7,968,146, PCT Pat. Application Nos. PCT/US2007/023098 and PCT/US2009/042829, which are herein incorporated by reference in their entireties. To be considered a “mixture”, the aforesaid polymeric and non-polymeric materials comprising the barrier layer should be deposited under the same reaction conditions and/or at the same time. The weight ratio of polymeric to non-polymeric material may be in the range of 95:5 to 5:95. The polymeric material and the non-polymeric material may be created from the same precursor material. In one example, the mixture of a polymeric material and a non-polymeric material consists essentially of polymeric silicon and inorganic silicon.

In some embodiments, at least one of the anode, the cathode, or a new layer disposed over the organic emissive layer functions as an enhancement layer. The enhancement layer comprises a plasmonic material exhibiting surface plasmon resonance that non-radiatively couples to the emitter material and transfers excited state energy from the emitter material to non-radiative mode of surface plasmon polariton. The enhancement layer is provided no more than a threshold distance away from the organic emissive layer, wherein the emitter material has a total non-radiative decay rate constant and a total radiative decay rate constant due to the presence of the enhancement layer and the threshold distance is where the total non-radiative decay rate constant is equal to the total radiative decay rate constant. In some embodiments, the OLED further comprises an outcoupling layer. In some embodiments, the outcoupling layer is disposed over the enhancement layer on the opposite side of the organic emissive layer. In some embodiments, the outcoupling layer is disposed on opposite side of the emissive layer from the enhancement layer but still outcouples energy from the surface plasmon mode of the enhancement layer. The outcoupling layer scatters the energy from the surface plasmon polaritons. In some embodiments this energy is scattered as photons to free space. In other embodiments, the energy is scattered from the surface plasmon mode into other modes of the device such as but not limited to the organic waveguide mode, the substrate mode, or another waveguiding mode. If energy is scattered to the non-free space mode of the OLED other outcoupling schemes could be incorporated to extract that energy to free space. In some embodiments, one or more intervening layer can be disposed between the enhancement layer and the outcoupling layer. The examples for intervening layer(s) can be dielectric materials, including organic, inorganic, perovskites, oxides, and may include stacks and/or mixtures of these materials.

The enhancement layer modifies the effective properties of the medium in which the emitter material resides resulting in any or all of the following: a decreased rate of emission, a modification of emission line-shape, a change in emission intensity with angle, a change in the stability of the emitter material, a change in the efficiency of the OLED, and reduced efficiency roll-off of the OLED device. Placement of the enhancement layer on the cathode side, anode side, or on both sides results in OLED devices which take advantage of any of the above-mentioned effects. In addition to the specific functional layers mentioned herein and illustrated in the various OLED examples shown in the figures, the OLEDs according to the present disclosure may include any of the other functional layers often found in OLEDs.

The enhancement layer can be comprised of plasmonic materials, optically active metamaterials, or hyperbolic metamaterials. As used herein, a plasmonic material is a material in which the real part of the dielectric constant crosses zero in the visible or ultraviolet region of the electromagnetic spectrum. In some embodiments, the plasmonic material includes at least one metal. In such embodiments the metal may include at least one of Ag, Al, Au, Ir, Pt, Ni, Cu, W, Ta, Fe, Cr, Mg, Ga, Rh, Ti, Ru, Pd, In, Bi, Ca alloys or mixtures of these materials, and stacks of these materials. In general, a metamaterial is a medium composed of different materials where the medium as a whole acts differently than the sum of its material parts. In particular, we define optically active metamaterials as materials which have both negative permittivity and negative permeability. Hyperbolic metamaterials, on the other hand, are anisotropic media in which the permittivity or permeability are of different sign for different spatial directions. Optically active metamaterials and hyperbolic metamaterials are strictly distinguished from many other photonic structures such as Distributed Bragg Reflectors (“DBRs”) in that the medium should appear uniform in the direction of propagation on the length scale of the wavelength of light. Using terminology that one skilled in the art can understand: the dielectric constant of the metamaterials in the direction of propagation can be described with the effective medium approximation. Plasmonic materials and metamaterials provide methods for controlling the propagation of light that can enhance OLED performance in a number of ways.

In some embodiments, the enhancement layer is provided as a planar layer. In other embodiments, the enhancement layer has wavelength-sized features that are arranged periodically, quasi-periodically, or randomly, or sub-wavelength-sized features that are arranged periodically, quasi-periodically, or randomly. In some embodiments, the wavelength-sized features and the sub-wavelength-sized features have sharp edges.

In some embodiments, the outcoupling layer has wavelength-sized features that are arranged periodically, quasi-periodically, or randomly, or sub-wavelength-sized features that are arranged periodically, quasi-periodically, or randomly. In some embodiments, the outcoupling layer may be composed of a plurality of nanoparticles and in other embodiments the outcoupling layer is composed of a plurality of nanoparticles disposed over a material. In these embodiments the outcoupling may be tunable by at least one of varying a size of the plurality of nanoparticles, varying a shape of the plurality of nanoparticles, changing a material of the plurality of nanoparticles, adjusting a thickness of the material, changing the refractive index of the material or an additional layer disposed on the plurality of nanoparticles, varying a thickness of the enhancement layer, and/or varying the material of the enhancement layer. The plurality of nanoparticles of the device may be formed from at least one of metal, dielectric material, semiconductor materials, an alloy of metal, a mixture of dielectric materials, a stack or layering of one or more materials, and/or a core of one type of material and that is coated with a shell of a different type of material. In some embodiments, the outcoupling layer is composed of at least metal nanoparticles wherein the metal is selected from the group consisting of Ag, Al, Au, Ir, Pt, Ni, Cu, W, Ta, Fe, Cr, Mg, Ga, Rh, Ti, Ru, Pd, In, Bi, Ca, alloys or mixtures of these materials, and stacks of these materials. The plurality of nanoparticles may have additional layer disposed over them. In some embodiments, the polarization of the emission can be tuned using the outcoupling layer. Varying the dimensionality and periodicity of the outcoupling layer can select a type of polarization that is preferentially outcoupled to air. In some embodiments the outcoupling layer also acts as an electrode of the device.

It is believed that the internal quantum efficiency (IQE) of fluorescent OLEDs can exceed the 25% spin statistics limit through delayed fluorescence. As used herein, there are two types of delayed fluorescence, i.e. P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the thermal population between the triplet states and the singlet excited states. Compounds that are capable of generating E-type delayed fluorescence are required to have very small singlet-triplet gaps. Thermal energy can activate the transition from the triplet state back to the singlet state. This type of delayed fluorescence is also known as thermally activated delayed fluorescence (TADF). A distinctive feature of TADF is that the delayed component increases as temperature rises due to the increased thermal energy. If the reverse intersystem crossing rate is fast enough to minimize the non-radiative decay from the triplet state, the fraction of back populated singlet excited states can potentially reach 75%. The total singlet fraction can be 100%, far exceeding the spin statistics limit for electrically generated excitons.

E-type delayed fluorescence characteristics can be found in an exciplex system or in a single compound. Without being bound by theory, it is believed that E-type delayed fluorescence requires the luminescent material to have a small singlet-triplet energy gap (ΔES-T). Organic, non-metal containing, donor-acceptor luminescent materials may be able to achieve this. The emission in these materials is often characterized as a donor-acceptor charge-transfer (CT) type emission. The spatial separation of the HOMO and LUMO in these donor-acceptor type compounds often results in small ΔES-T. These states may involve CT states. Often, donor-acceptor luminescent materials are constructed by connecting an electron donor moiety such as amino- or carbazole-derivatives and an electron acceptor moiety such as N-containing six-membered aromatic ring.

Devices fabricated in accordance with embodiments of the invention can be incorporated into a wide variety of electronic component modules (or units) that can be incorporated into a variety of electronic products or intermediate components. Examples of such electronic products or intermediate components include display screens, lighting devices such as discrete light source devices or lighting panels, etc. that can be utilized by the end-user product manufacturers. Such electronic component modules can optionally include the driving electronics and/or power source(s). Devices fabricated in accordance with embodiments of the invention can be incorporated into a wide variety of consumer products that have one or more of the electronic component modules (or units) incorporated therein. A consumer product comprising an OLED that includes the compound of the present disclosure in the organic layer in the OLED is disclosed. Such consumer products would include any kind of products that include one or more light source(s) and/or one or more of some type of visual displays. Some examples of such consumer products include a flat panel display, a curved display, a computer monitor, a medical monitor, a television, a billboard, a light for interior or exterior illumination and/or signaling, a heads-up display, a fully or partially transparent display, a flexible display, a rollable display, a foldable display, a stretchable display, a laser printer, a telephone, a cell phone, tablet, a phablet, a personal digital assistant (PDA), a wearable device, a laptop computer, a digital camera, a camcorder, a viewfinder, a micro-display that is less than 2 inches diagonal, a 3-D display, a virtual reality or augmented reality display, a vehicle, a video walls comprising multiple displays tiled together, a theater or stadium screen, and a sign. Various control mechanisms may be used to control devices fabricated in accordance with the present invention, including passive matrix and active matrix. Many of the devices are intended for use in a temperature range comfortable to humans, such as 18° C. to 30° C., and more preferably at room temperature (20-25 C), but could be used outside this temperature range, for example, from −40° C. to 80° C.

The materials and structures described herein may have applications in devices other than OLEDs. For example, other optoelectronic devices such as organic solar cells and organic photodetectors may employ the materials and structures. More generally, organic devices, such as organic transistors, may employ the materials and structures.

The present disclosure relates to OLEDs that include an emissive region comprising at least two different metal complexes. The different metal complexes may comprise the same or different metals. In particular, a palladium complexes may serve as a host, emitter, or a dopant in conjunction with another metal complex in the emissive region of an OLED.

In general, Pd-complexes are harder to oxidize than their Pt analogs making them less thermodynamically favorable hole traps or recombination centers in an OLED. This property limits their utility as an emitting material. However, it has been determined that the combination of a Pd-complex with another phosphor, such as an Ir or Pt complex, can have a beneficial effect on device performance. For example, in the emissive regions described herein, the Pd-complex can serve as an intermediate energy level to facilitate charge transport or could serve as a high triplet energy T₁ host material in a device.

In one aspect, an OLED comprising an anode; an cathode emissive region; and a cathode is provided. The emissive region can be disposed between the anode and the. The emissive region can include a first compound, D1, which is a first metal complex; and a second compound, D2, which is a second metal complex, where D1 and D2 are different. The emission from the OLED does not primarily originate from D2 or from an exciplex between D1 and D2, the emission from the OLED has an emission peak maximum between 300 to 2000 nm; and, when the emission from the OLED has a peak between 550 and 700 nm, then D2 is not a Be, Al, or Ir complex.

As used herein, exciplex is defined between a first molecule D and a second molecule A, wherein the first molecule D has a HOMO energy of HOMO_{D}, a LUMO energy of LUMO_{D}, and a lowest triplet energy of T1_{D}; the second molecule A has a HOMO energy of HOMO_{A}, a LUMO energy of LUMO_{A}, and a lowest triplet energy of T1_{A}; wherein |HOMO_{D}|<|HOMO_{A}|, |LUMO_{D}|<|LUMO_{A}|, |HOMO_{D}−LUMO_{A}|<T1_{D}, and |HOMO_{D}−LUMO_{A}|<T1_{A}. The HOMO and LUMO energies are measured by solution electrochemistry as described herein. The lowest triplet energy T1 can be obtained from the short-wavelength band of a phosphorescence spectrum by determining the wavelength at which the onset of the shortest-wavelength peak of the phosphorescence spectrum achieves 10% of the intensity of the peak of the gated emission of a frozen sample in 2-MeTHF at 77 K.

In some embodiments, |HOMO_{D}| is less than |HOMO_{A}| by at least 10, 20, 50, 100, 150, 200, 250, or 300 meV. In some embodiments, |LUMO_{D}| is less than |LUMO_{A}| by at least 10, 20, 50, 100, 150, 200, 250, or 300 meV. In some embodiments, |HOMO_{D}−LUMO_{A}| is less than T1_{D} and/or T1_{A} by at least 10, 20, 50, 100, 150, 200, 250, or 300 meV. When the exciplex is placed in a matrix system, such as in an EML, the emission from the exciplex may be observable or may not be observable.

In some embodiments, the emission from D2 is less than 10% of the emission from the OLED. In some embodiments, the emission from the OLED has a peak between 550 and 700 nm, and D2 is not a Pt complex. For instance, in some embodiments, if the emission from the OLED has a peak between 550 and 700 nm, then D2 is not a Pt complex.

In some embodiments, D2 comprises a metal M2 selected from the group consisting of Ir, Pt, Pd, Au, Ag, Cu, Zn, Zr, Ru, Re, Co, Ti, Os, Rh, Be, Mn, Mg, Ni, Fe, Tl, Pb, Po, At, Sb, Te, Al, Ga, In, Bi, and Sn. In some embodiments, D2 is a Pt complex. In some embodiments, D2 is a Pd complex.

In some embodiments, D1 comprises a metal M1 selected from the group consisting of Ir, Os, Rh, Pt, Pd, Au, Ag, and Cu. In some embodiments, D1 is an Ir complex. In some embodiments, D1 is a Pt complex.

In some embodiments, D1 is an Ir complex, and D2 is a Pd complex. In some embodiments, D1 is a Pt complex, and D2 is a Pd complex.

In some embodiments, M1 and M2 are the same. In some embodiments, M1 and M2 are different.

In some embodiments, D1 has at least one ligand with the same structure as a ligand in D2.

In some embodiments, each ligand in D1 has the same structure as the corresponding ligand in D2. In some such embodiments, D1 is a Pt complex, and D2 is a Pd complex.

In some embodiments, at least one ligand of D1 is not the same as at least one ligand in D2.

In some embodiments, the emissive region comprises more than one layer. In some such embodiments, D1 and D2 are in the same layer of the emissive region. In some embodiments, D1 and D2 are not in the same layer of the emissive region.

In some embodiments, D1 is responsible for at least 70% of the emissions from the OLED.

In some embodiments, D1 is responsible for at least 80% of the emissions from the OLED.

In some embodiments, D1 is responsible for at least 90% of the emissions from the OLED.

In some embodiments, D1 is responsible for at least 99% of the emissions from the OLED. In some embodiments, D2 is less than 30%. In some embodiments, D2 is less than 20%. In some embodiments, D2 is less than 15%. In some embodiments, D2 is less than 10%. In some embodiments, D2 is less than 5%. In some embodiments, D2 is less than 1%.

In some embodiments, D2 is a host material.

In some embodiments where D2 is a host material, D2 has the formula of M(L¹)ₓ(L²)_{y}(L³)_{z};
wherein L¹, L², and L³ can be the same or different;
wherein x is 1, 2, or 3;
wherein y is 0, 1, or 2;
wherein z is 0, 1, or 2;
wherein x+y+z is the oxidation state of the metal M;
wherein L¹ is selected from the group consisting of the structures of the following LIST 1:

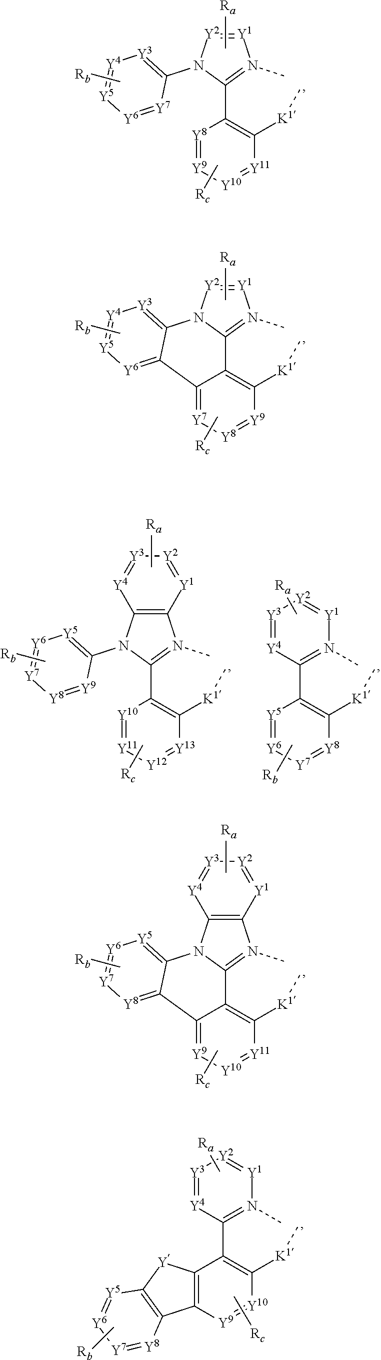

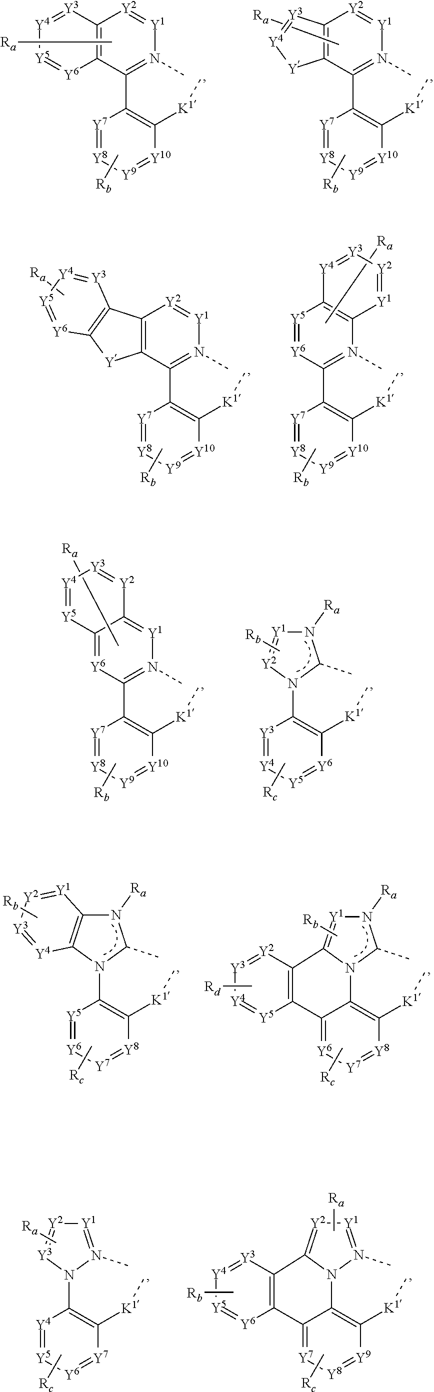

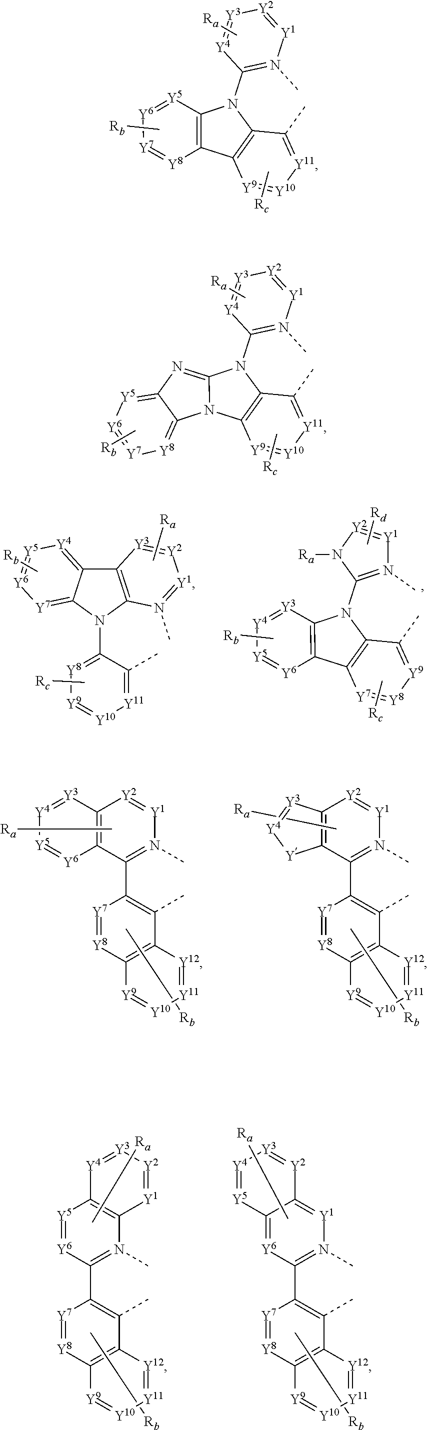

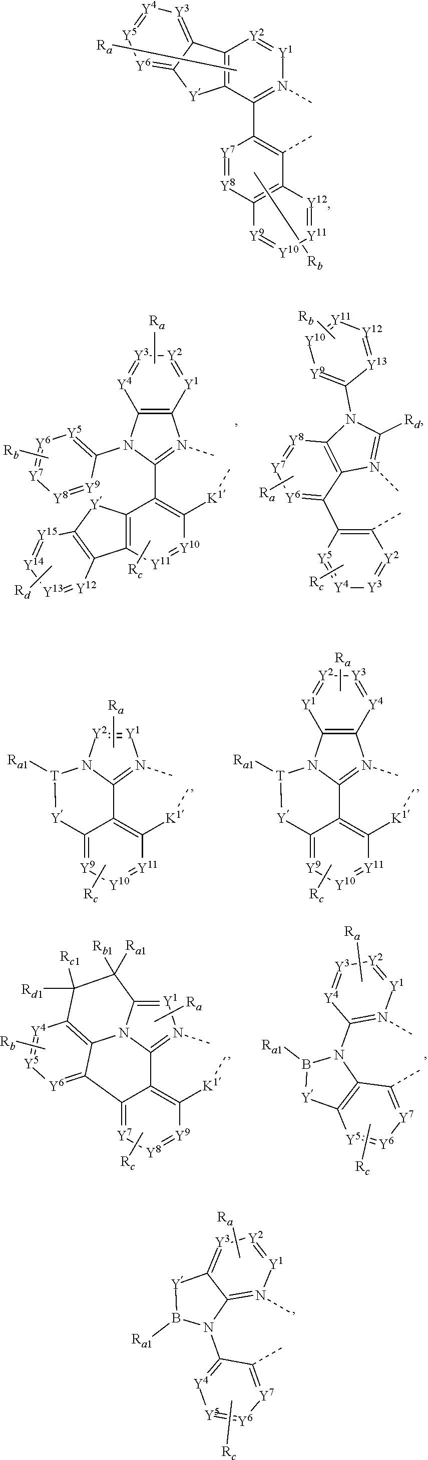

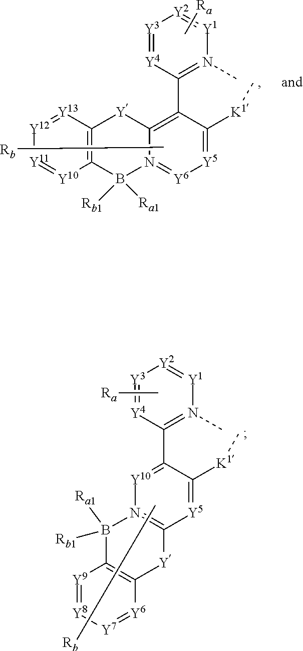

wherein L² and L³ are independently selected from the group consisting of

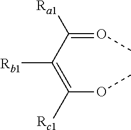

and the structures of LIST 1 defined herein;
   wherein:
T is selected from the group consisting of B, Al, Ga, and In;
K^{1′} is a direct bond or is selected from the group consisting of NRₑ, PRₑ, O, S, and Se;
each Y¹ to Y¹³ are independently selected from the group consisting of carbon and nitrogen;
Y′ is selected from the group consisting of BRₑ, NRₑ, P Rₑ, O, S, Se, C═O, S═O, SO₂, CRₑR_{f}, SiRₑR_{f}, and GeRₑR_{f};
Rₑ and R_{f} can be fused or joined to form a ring;
each Rₐ, R_{b}, R_{c}, and R_{d} can independently represent from mono to the maximum possible number of substitutions, or no substitution;
each Rₐ₁, R_{b1}, R_{c1}, R_{d1}, Rₐ, R_{b}, R_{c}, R_{d}, Rₑ, and R_{f} is independently a hydrogen or a substituent selected from the group consisting of deuterium, halogen, alkyl, cycloalkyl, heteroalkyl, heterocycloalkyl, boryl, arylalkyl, alkoxy, aryloxy, amino, silyl, germyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof; and
any two adjacent substituents of Rₐ₁, R_{b1}, R_{c1}, R_{d1}, Rₐ, R_{b}, R_{c}, and R_{d} can be fused or joined to form a ring or form a multidentate ligand.

In some embodiments where D2 is a host material, D2 has a structure of Formula I:

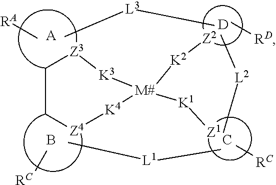

wherein:
M^{#} is Pd or Pt;
moieties A, B, C, and D are each independently monocyclic or polycyclic ring structure comprising 5-membered and/or 6-membered carbocyclic or heterocyclic rings;
Z¹, Z², Z³, and Z⁴ are each independently C or N;
K¹, K², K³, and K⁴ are each independently selected from the group consisting of a direct bond, O, and S, wherein at least two of K¹, K², K³, and K⁴ are direct bonds;
L¹, L², and L³ are each independently selected from the group consisting of absent a bond, a direct bond, BR, BRR′, NR, PR, P(O)R, O, S, Se, C═O, C═S, C═Se, C═NR, C═CRR′, S═O, SO₂, CR, CRR′, SiRR′, GeRR′, alkylene, cycloalkyl, aryl, cycloalkylene, arylene, heteroarylene, and combinations thereof, wherein at least one of L¹ and L² is present;
R^{A}, R^{B}, R^{C} and R^{D} each independently represents zero, mono, or up to a maximum allowed number of substitutions to its associated ring;
each of R, R′, R^{A}, R^{B}, R^{C}, and R^{D} is independently a hydrogen or a substituent selected from the group consisting of deuterium, fluorine, alkyl, cycloalkyl, heteroalkyl, alkoxy, aryloxy, amino, silyl, boryl, alkenyl, cycloalkenyl, heteroalkenyl, aryl, heteroaryl, nitrile, isonitrile, sulfanyl, and combinations thereof; and
any two R, R′, R^{A}, R^{B}, R^{C}, R^{E}, and R^{F} can be joined or fused together to form a ring.

In some such embodiments, each of moieties A, B, C, and D is independently selected from the group consisting of benzene, pyridine, pyrimidine, pyridazine, pyrazine, triazine, imidazole, pyrazole, pyrrole, oxazole, furan, thiophene, triazole, thiazole, naphthalene, quinoline, isoquinoline, quinazoline, benzofuran, benzoxazole, benzothiophene, benzothiazole, benzoselenophene, indene, indole, benzimidazole, carbazole, dibenzofuran, dibenzothiophene, quinoxaline, phthalazine, phenanthrene, phenanthridine, and fluorene.

In some embodiments, D2 may have a structure of Formula II or Formula III:

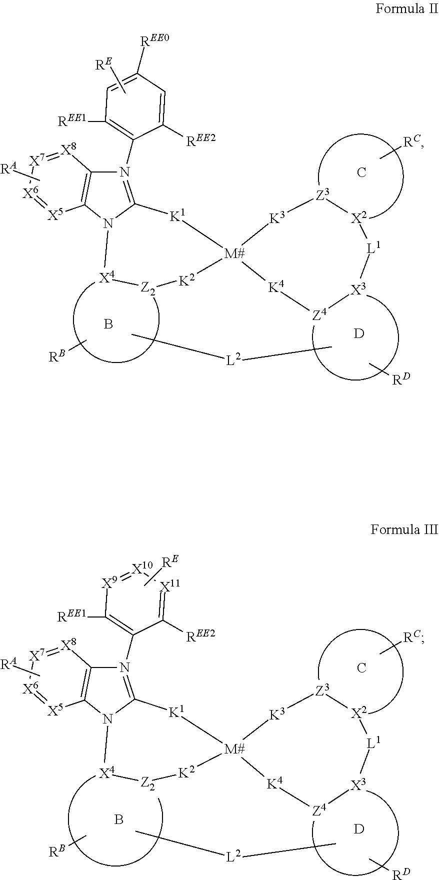

wherein:
X⁵ to X¹¹ are each independently C or N;
R^{E} represents mono to the maximum allowable substitutions, or no substitutions;
each of R^{E}, R^{EE0}, R^{EE1} and R^{EE2} is independently hydrogen or a substituent selected from the group consisting of the General Substituents defined herein; and
any two substituents may be joined or fused to form a ring.

In some embodiments, no R^{E} is joined or fused with R^{EE1} or R^{EE2} to form a ring.

In some embodiments, R^{EE0} is selected from the group consisting of halogen, alkyl, cycloalkyl, heteroalkyl, heterocycloalkyl, arylalkyl, alkoxy, aryloxy, amino, silyl, germyl, boryl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof. In some embodiments, R^{EE0} is not H or D. In some embodiments, R^{EE0} is alkyl, cycloalkyl, aryl, or heteroaryl. In some embodiments, R^{EE0} is C₆H₅, C₆D₅, C(CH₃)₃, C(CD₃)₃, CD₂C(CH₃)₃, CH₃, CD₃, cyclopentyl, cyclohexyl, or neopentyl.

In some embodiments, X⁵ to X¹¹ are each C. In some embodiments, one of X⁵ to X¹¹ is N. In some embodiments, two of X⁵ to X¹¹ are N. In some embodiments, one of X⁵ to X⁸ is N. In some embodiments, one of X⁹ and X¹¹ is N. In some embodiments, X¹⁰ is N.

In some embodiments, R^{EE1} is the same as R^{EE2}. In some embodiments, R^{EE1} is different from R^{EE2}.

In some embodiments, at least one of R^{EE1} or R^{EE2} comprises a chemical group containing at least three 6-membered aromatic rings that are not fused next to each other. In some embodiments, at least one of R^{EE1} or R^{EE2} comprises a chemical group containing at least four 6-membered aromatic rings that are not fused next to each other. In some embodiments, at least one of R^{EE1} or R^{EE2} comprises a chemical group containing at least five 6-membered aromatic rings that are not fused next to each other. In some embodiments, at least one of R^{EE1} or R^{EE2} comprises a chemical group containing at least six 6-membered aromatic rings that are not fused next to each other. In some embodiments, each of R^{EE1} and R^{EE2} independently comprises a chemical group containing at least three to six 6-membered aromatic rings that are not fused next to each other.

In some embodiments, at least one of R^{EE1} or R^{EE2} comprises a group R^{W}, where R^{W} has a structure selected from the group consisting of: Formula IIIA, ---Q^{A}(R^{1a})(R^{2a})ₐ(R^{3a})_{b}, Formula IIIB,

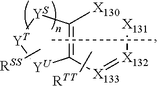

### and Formula IIIC,

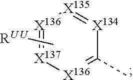

wherein:
each of X¹³⁰ to X¹³⁸ is independently C or N;
each of Y^{S}, Y^{T}, and Y^{U} is independently CRR′, SiRR′ or GeRR′;
n is an integer from 1 to 8,
when n is more than 1, each Y^{S} can be same or different;
Q^{A} is selected from the group consisting of C, Si, Ge, N, P, O, S, Se, and B;
   each of a and b is independently 0 or 1;
   if Q^{A} is C, Si, or Ge, then a+b=2;
   if Q^{A} is N or P, then a+b=1;
if Q^{A} is B, then a+b can be 1 or 2;
if Q^{A} is O, S, or Se, then a+b=0;
each of R^{SS}, R^{TT}, and R^{UU} independently represents mono to the maximum allowable number of substitutions, or no substitution;
each R, R′, R^{1a}, R^{2a}, R^{3a}, R^{SS}, R^{TT}, and R^{UU} is independently hydrogen or a substituent selected from the group consisting of the General Substituents defined herein; and
any two substituents may be optionally fused or joined to form a ring.

In some embodiments, at least one Y^{S}, Y^{T}, or Y^{U} is SiRR′ or GeRR′. In some embodiments, each Y^{S}, Y^{T}, and Y^{U} is CRR′.

In some embodiments, at least one of R^{EE1} and R^{EE2} comprises a group R^{W}. In some embodiments, each of R^{EE1} and R^{EE2} comprises a group R^{W}. In some embodiments, each of R^{EE1} and R^{EE2} comprises Formula IIIA. In some embodiments, each of R^{EE1} and R^{EE2} comprises Formula IIIB. In some embodiments, each of R^{EE1} and R^{EE2} comprises Formula IIIC. In some embodiments, either R^{EE1} or R^{EE2} comprises Formula IIIA, and the other one of R^{EE1} and R^{EE2} comprises Formula IIIB. In some embodiments, either R^{EE1} or R^{EE2} comprises Formula IIIA, and the other one of R^{EE1} and R^{EE2} comprises Formula IIIC. In some embodiments, either R^{EE1} or R^{EE2} comprises Formula IIIB, and the other one of R^{EE1} and R^{EE2} comprises Formula IIIC.

In some embodiments, R^{EE1} has a molecular weight (MW) greater than 15 g/mol and R^{EE2} has a molecular weight greater than that of R^{EE1}. In some embodiments, R^{EE1} has a molecular weight (MW) greater than 56 g/mol and R^{EE2} has a molecular weight greater than that of R^{EE1}. In some embodiments, R^{EE1} has a molecular weight (MW) greater than 76 g/mol and R^{EE2} has a molecular weight greater than that of R^{EE1} In some embodiments, R^{EE1} has a molecular weight (MW) greater than 81 g/mol and R^{EE2} has a molecular weight greater than that of R^{EE1}. In some embodiments, R^{EE1} or R^{EE2} has a molecular weight (MW) greater than 165 g/mol. In some embodiments, R^{EE1} or R^{EE2} has a molecular weight (MW) greater than 166 g/mol. In some embodiments, R^{EE1} or R^{EE2} has a molecular weight (MW) greater than 182 g/mol.

In some embodiments, R^{EE1} has one more 6-membered aromatic ring than R^{EE2}. In some embodiments, R^{EE1} has two more 6-membered aromatic rings than R^{EE2}. In some embodiments, R^{EE1} has three more 6-membered aromatic rings than R^{EE2}. In some embodiments, R^{EE1} has four more 6-membered aromatic rings than R^{EE2}. In some embodiments, R^{EE1} has five more 6-membered aromatic rings than R^{EE2}.

In some embodiments, R^{EE1} comprises at least one heteroatom and R^{EE2} consists of hydrocarbon and deuterated variant thereof. In some embodiments, R^{EE1} comprises at least two heteroatoms and R^{EE2} consists of hydrocarbon and deuterated variant thereof. In some embodiments, R^{EE1} comprises at least three heteroatoms and R^{EE2} consists of hydrocarbon and deuterated variant thereof. In some embodiments, R^{EE1} comprises exactly one heteroatom and R^{EE2} consists of hydrocarbon and deuterated variant thereof. In some embodiments, R^{EE1} comprises exactly two heteroatoms and R^{EE2} consists of hydrocarbon and deuterated variant thereof. In some embodiments, R^{EE1} comprises exactly three heteroatoms and R^{EE2} consists of hydrocarbon and deuterated variant thereof. In some embodiments, R^{EE1} comprises exactly one heteroatom and R^{EE2} comprises exactly one heteroatom that is different from the heteroatom in R^{EE1}. In some embodiments, R^{EE1} comprises exactly one heteroatom and R^{EE2} comprises exactly one heteroatom that is same as the heteroatom in R^{EE1}.

In some embodiments, R^{EE1} comprises exactly two heteroatoms and R^{EE2} comprises exactly one heteroatom. In some embodiments, R^{EE1} comprises exactly two heteroatoms and R^{EE2} comprises exactly two heteroatoms. In some embodiments, R^{EE1} comprises exactly three heteroatoms and R^{EE2} comprises exactly one heteroatom. In some embodiments, R^{EE1} comprises exactly three heteroatoms and R^{EE2} comprises exactly two heteroatoms. In some embodiments, R^{EE1} comprises exactly three heteroatoms and R^{EE2} comprises exactly three heteroatoms.

In some embodiments, at least one of R^{EE1} and R^{EE2} comprises an aromatic ring fused to a non-aromatic ring. In some embodiments, both R^{EE1} and R^{EE2} comprise an aromatic ring fused to a non-aromatic ring. In some embodiments, the aromatic ring is a phenyl ring and the non-aromatic ring is a cycloalkyl ring.

In some embodiments, at least one of R^{EE1} and R^{EE2} is partially or fully deuterated. In some embodiments, both R^{EE1} and R^{EE2} are partially or fully deuterated.

In some embodiments, one of the R^{EE1} and R^{EE2} is joined with R^{A} to form a cyclic ring. In some embodiments, D1 may have a structure of Formula IV or Formula V:

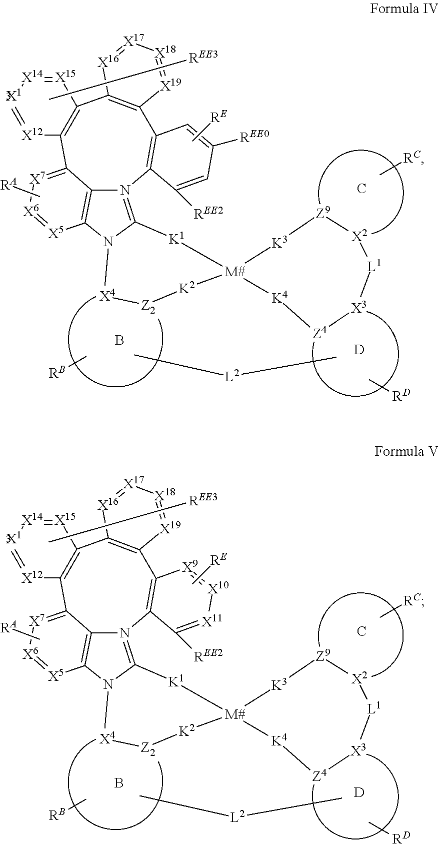

wherein
X¹² to X¹⁹ are each independently C or N; R^{EE3} is independently hydrogen or a substituent selected from the group consisting of the General Substituents defined herein; and any two substituents may be joined or fused to form a ring.

In some embodiments, X¹² to X¹⁹ are each C. In some embodiments, one of X¹² to X¹⁹ is N. In some embodiments, two of X¹² to X¹⁹ are N. In some embodiments, one of X¹² to X¹⁵ is N. In some embodiments, one of X¹⁶ to X¹⁹ is N.

In some embodiments where D2 is a host material, D2 has a structure selected from the group consisting of the structures of the following LIST 2:

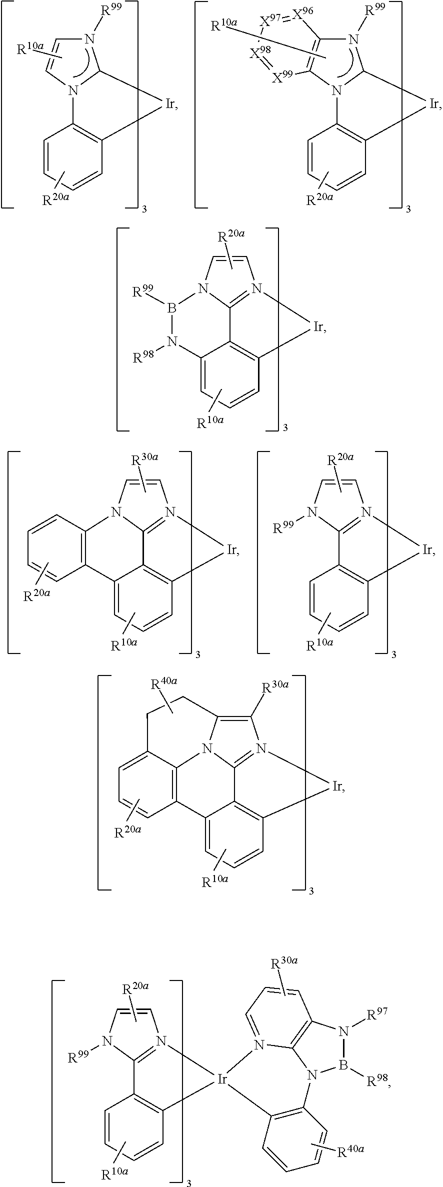

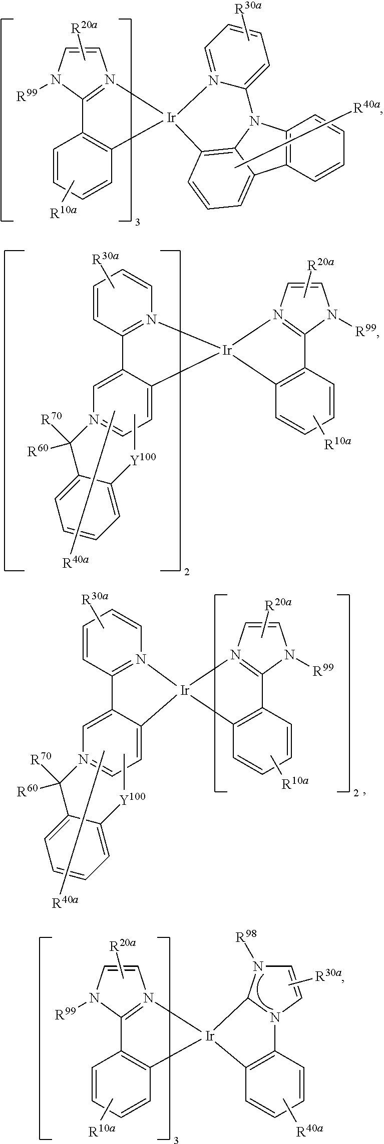

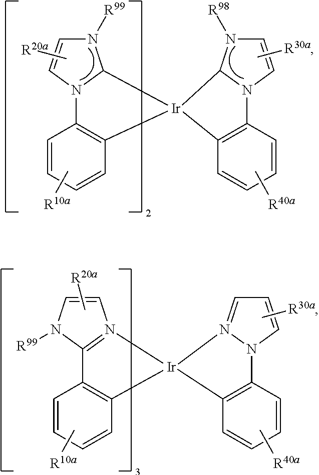

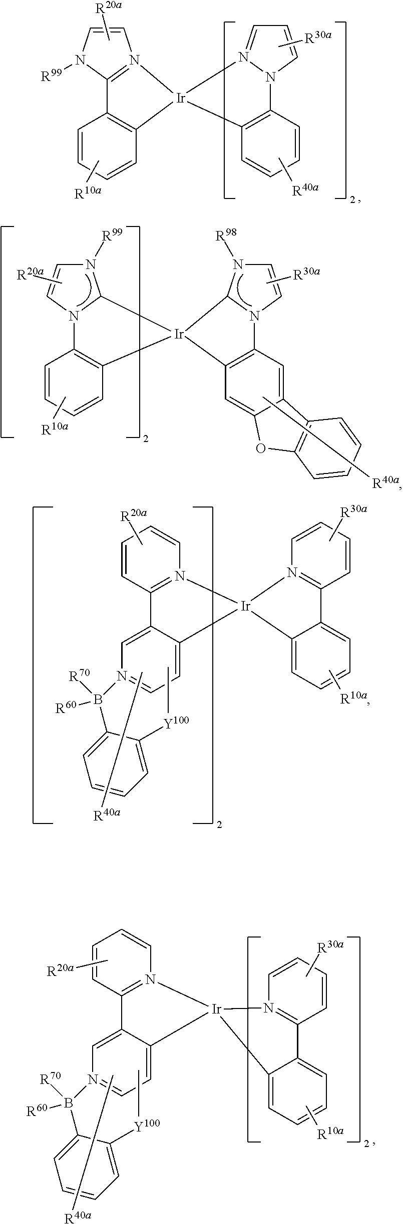

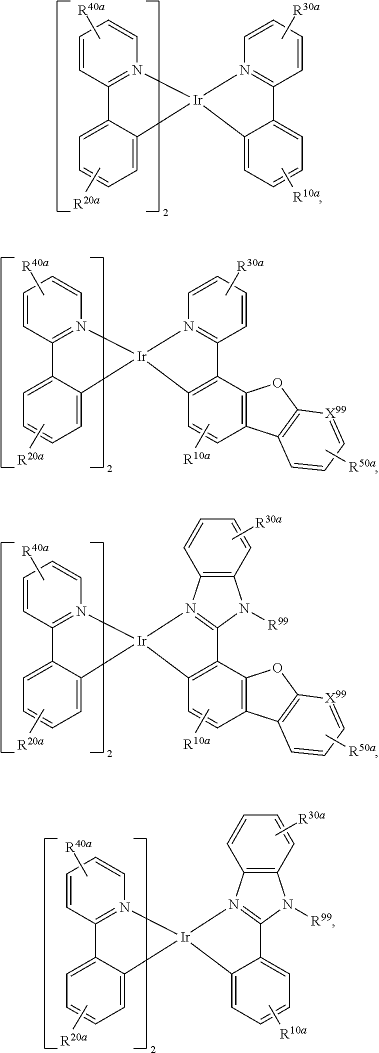

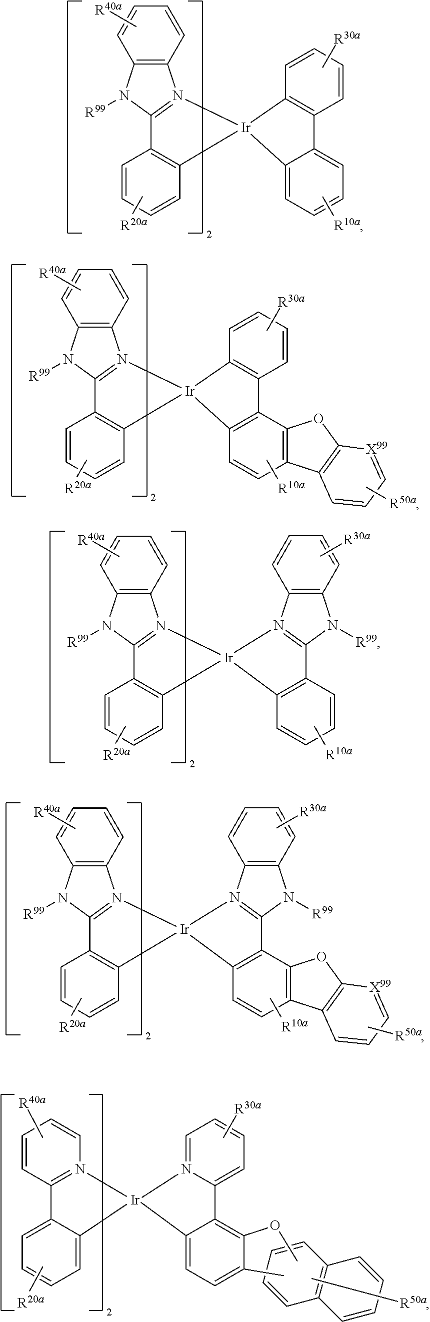

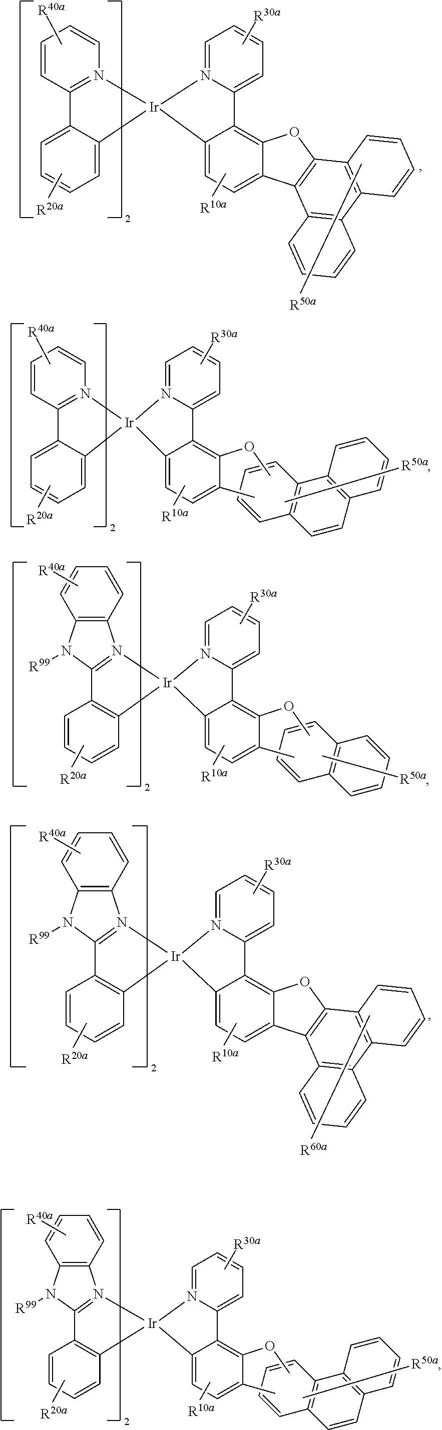

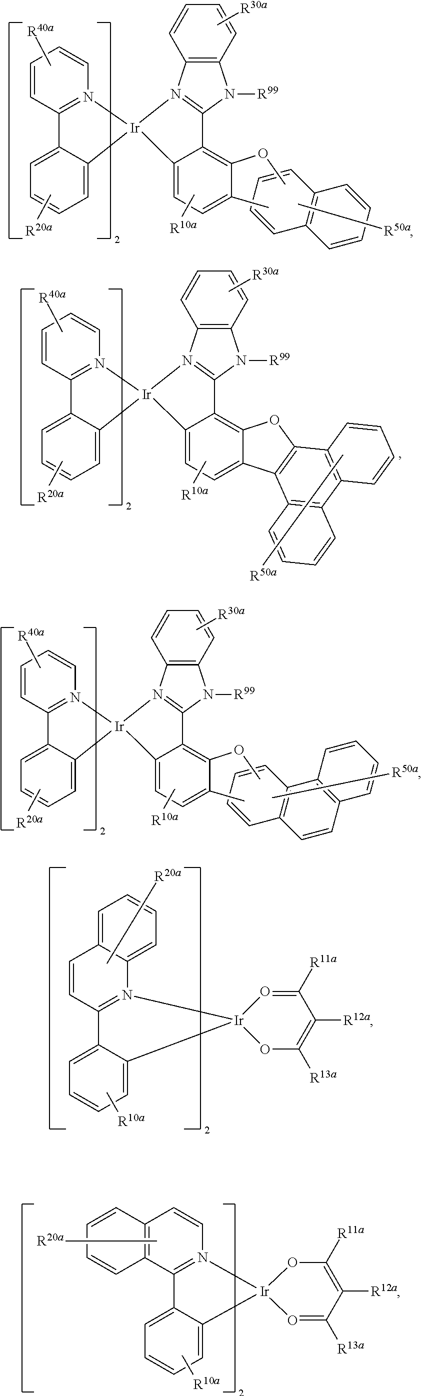

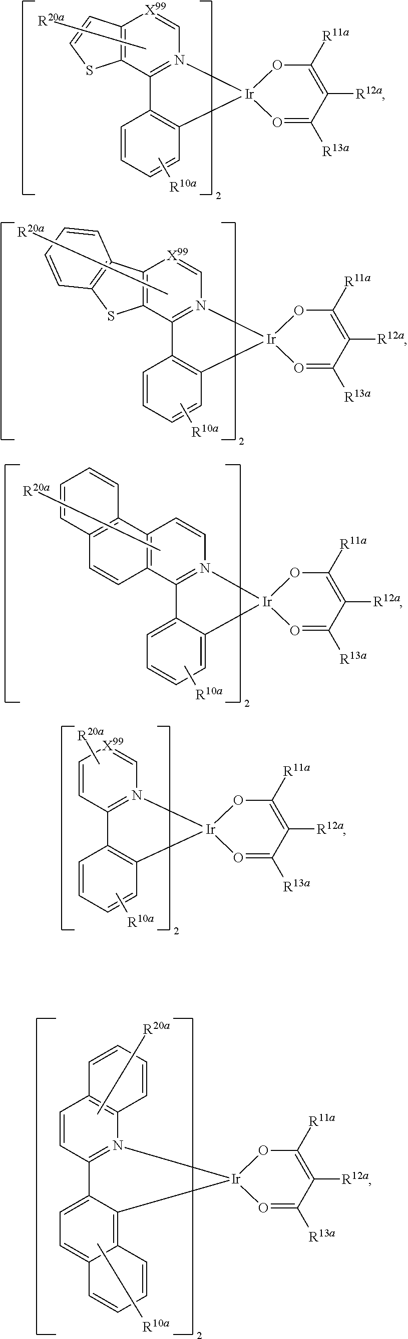

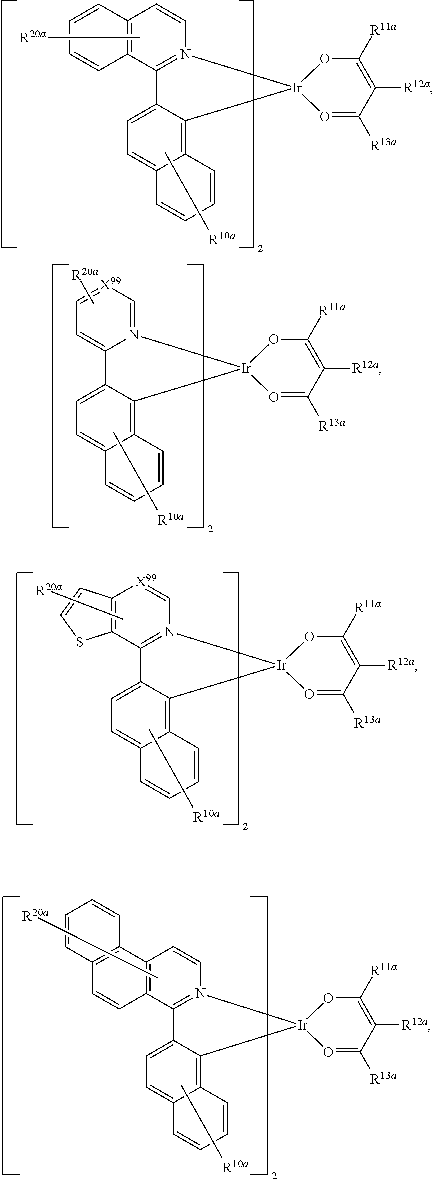

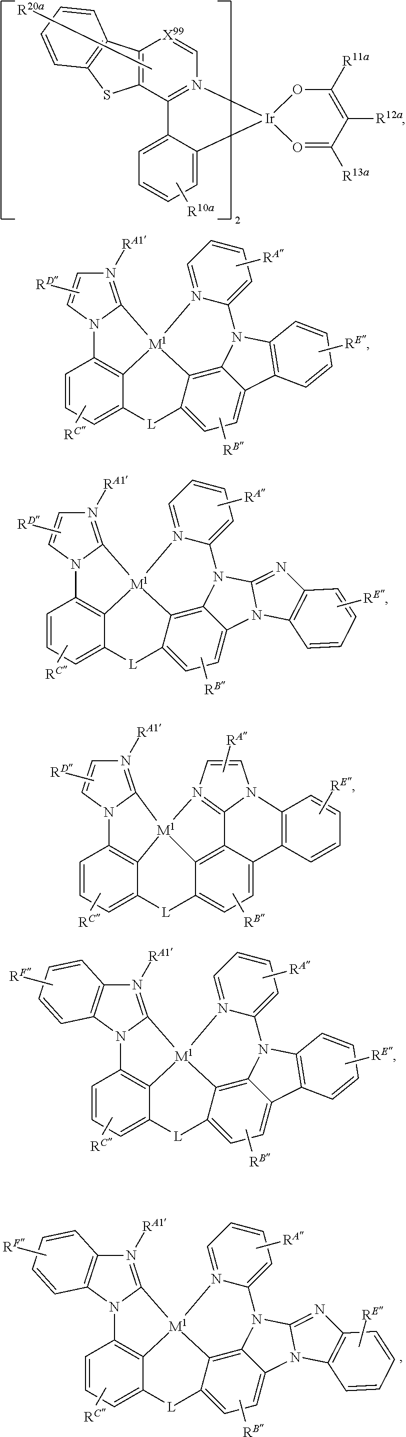

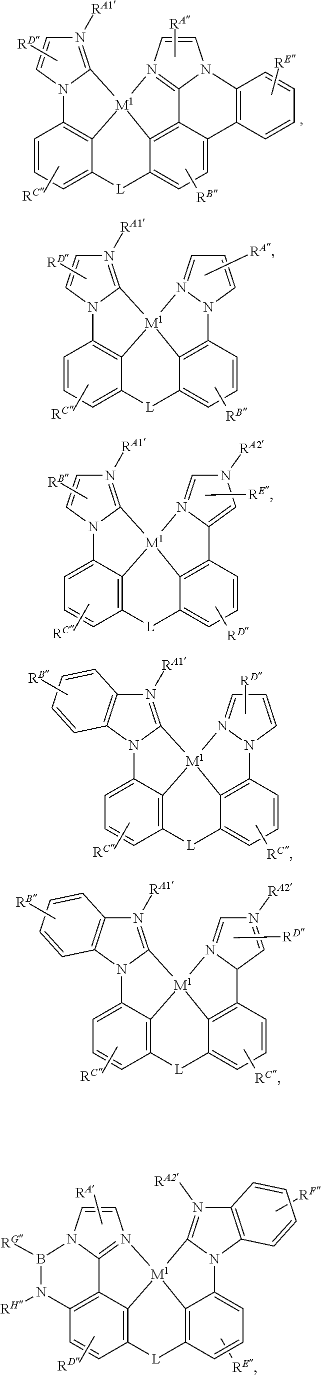

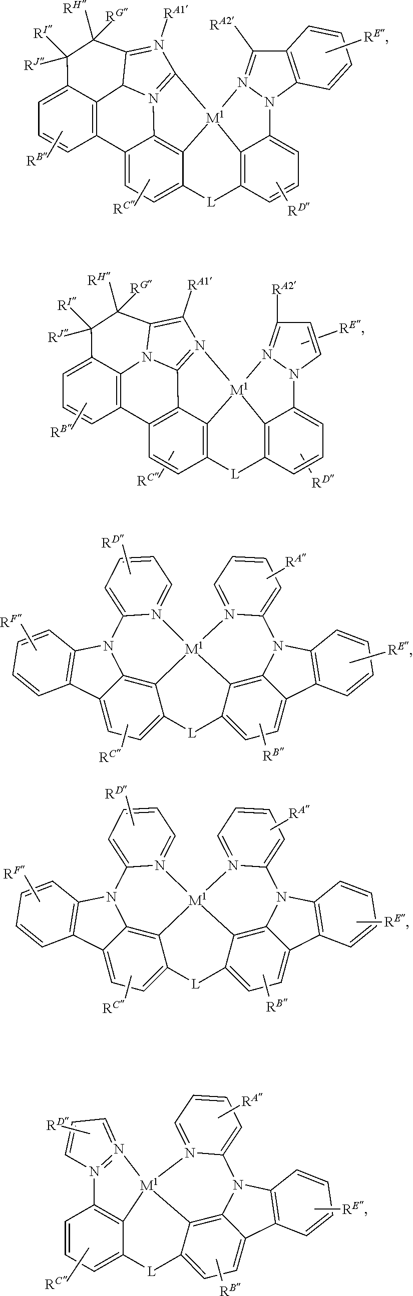

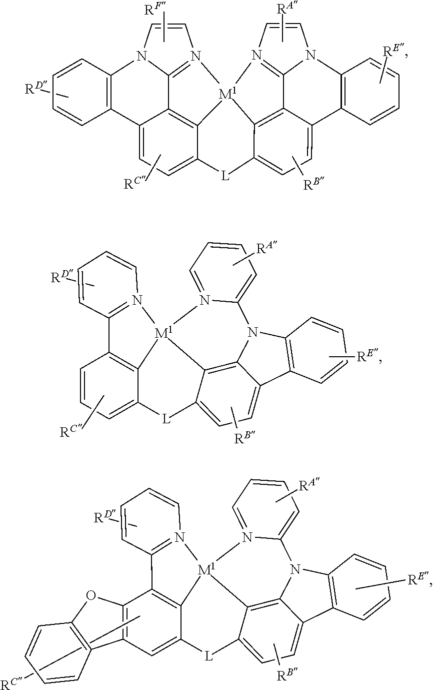

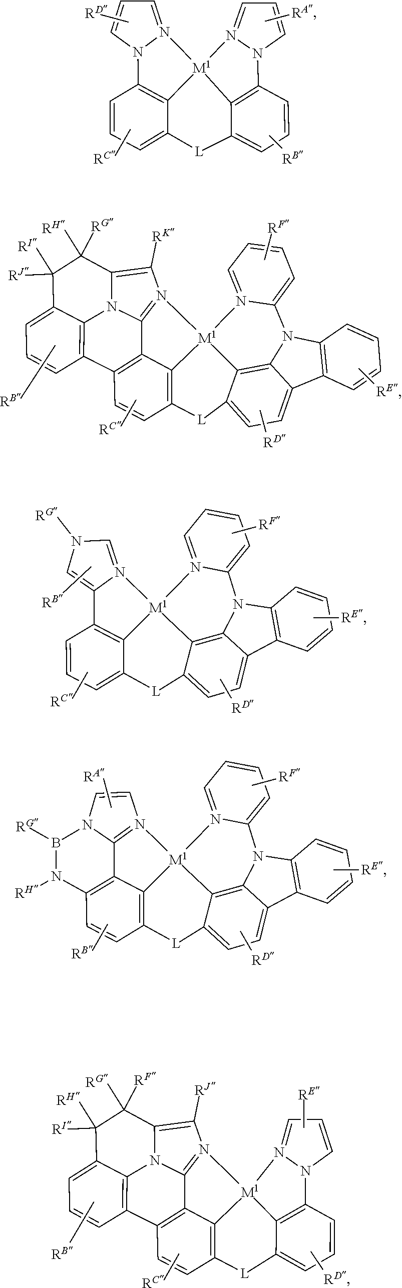

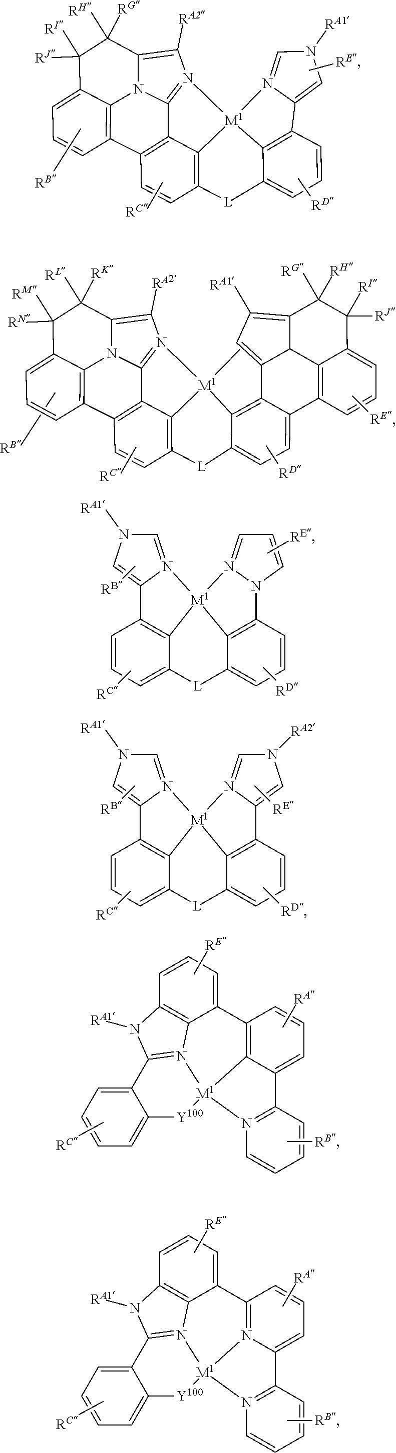

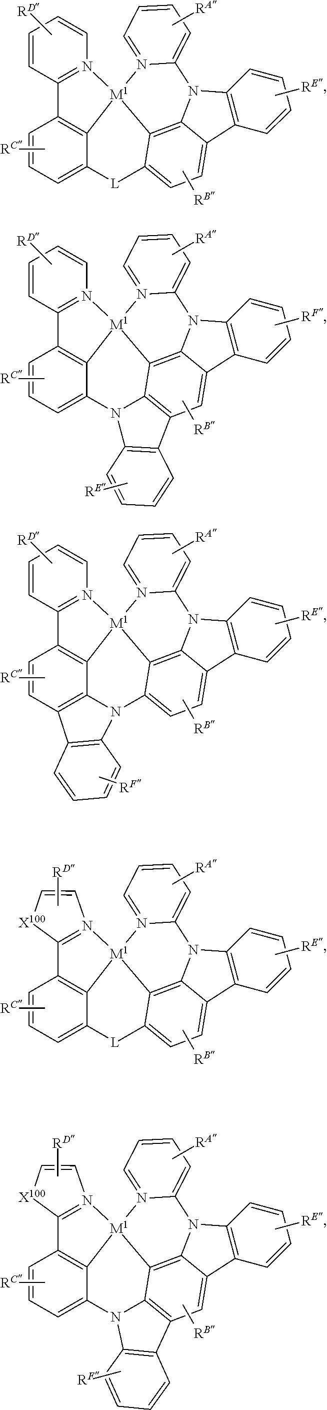

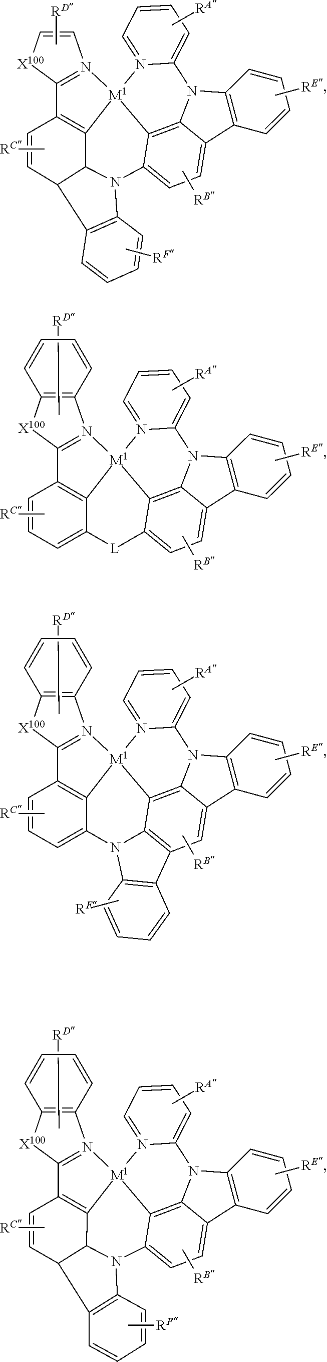

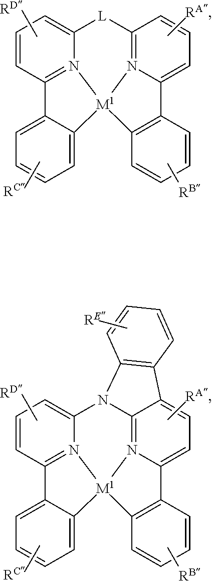

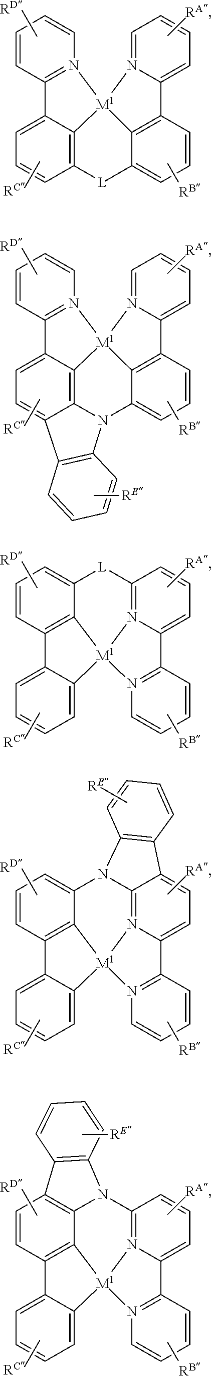

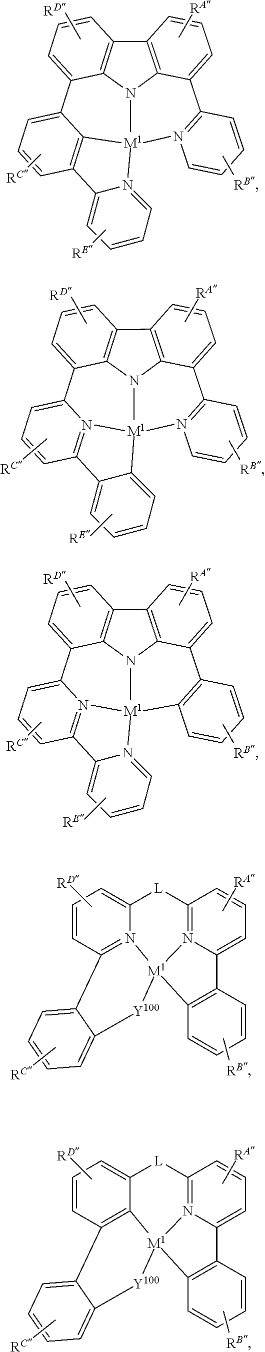

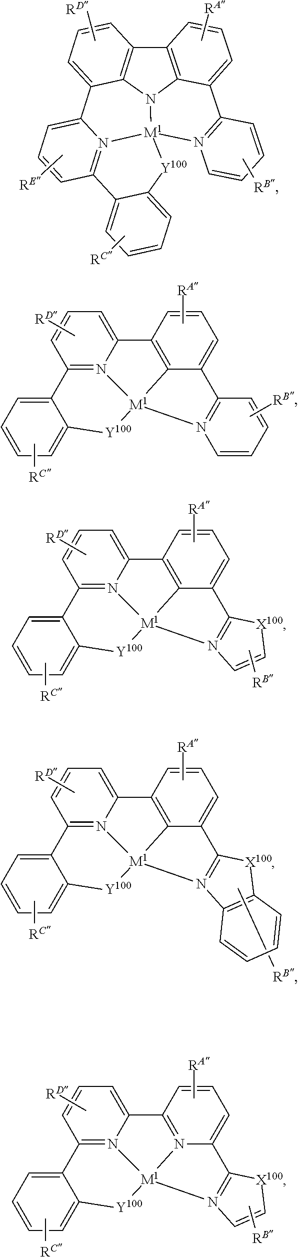

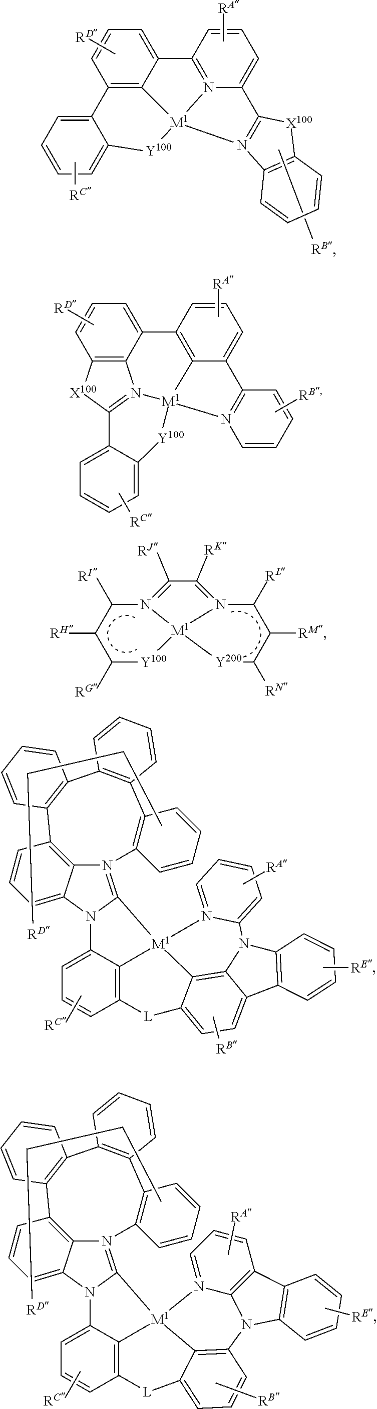

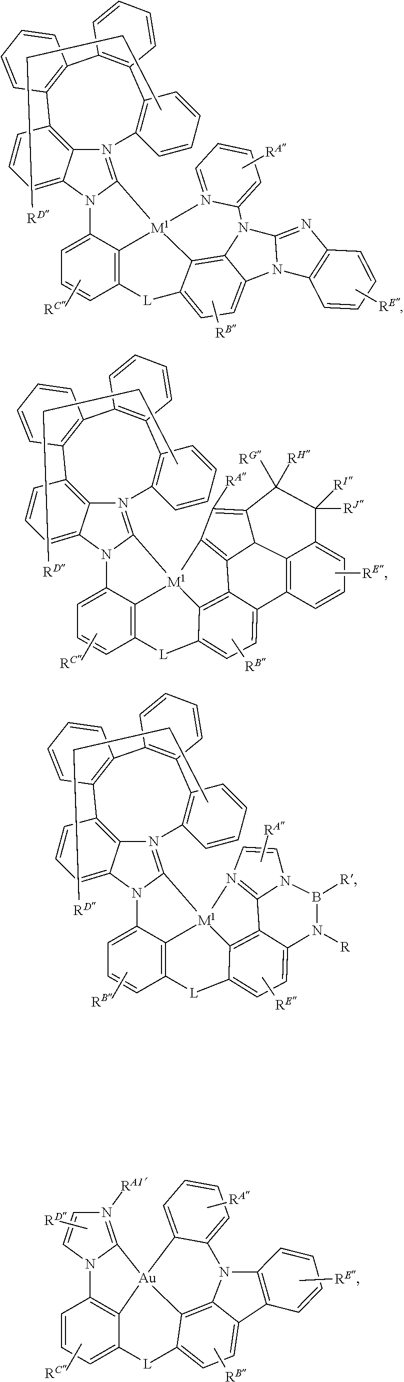

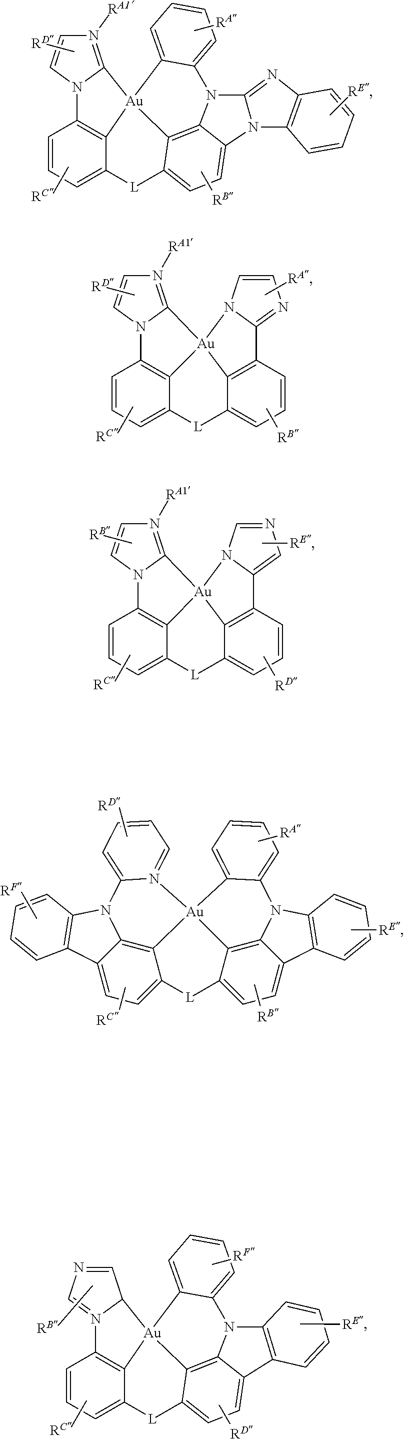

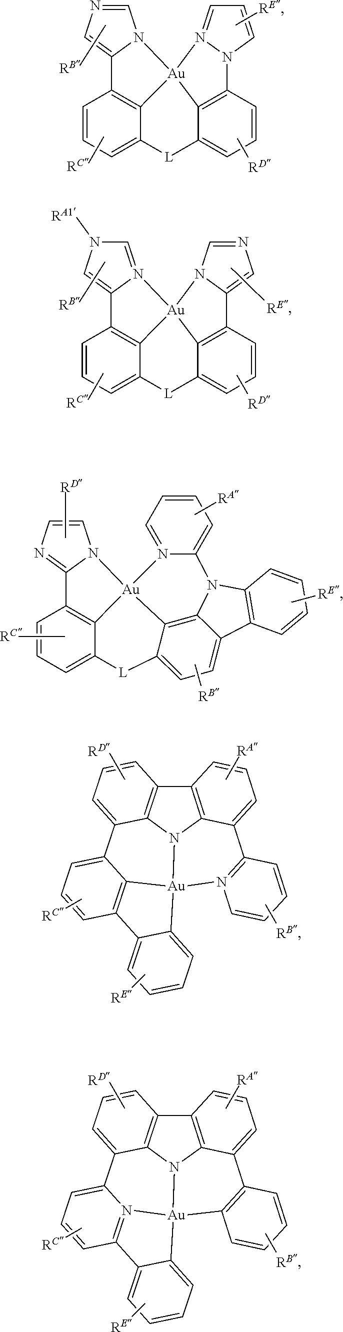

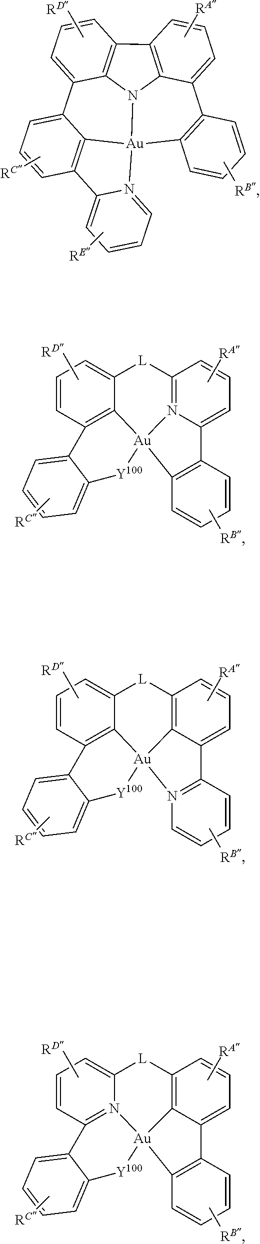

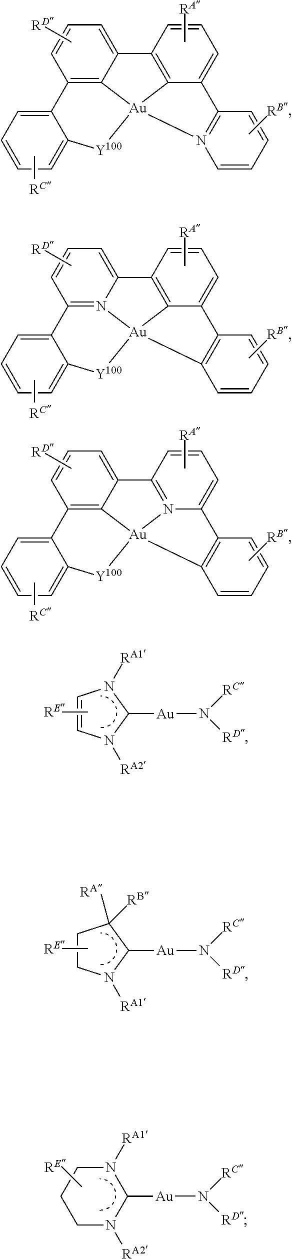

wherein:
M1 is selected from the group consisting of Pt, Pd, and Ni;
each of X⁹⁶ to X⁹⁹ is independently C or N;
each of Y¹⁰⁰ and Y²⁰⁰ is independently selected from the group consisting of a NR″, O, S, and Se;
L is independently selected from the group consisting of a direct bond, BR″, BR″R′″, NR″, PR″, O, S, Se, C═O, C═S, C═Se, C═NR″, C═CR″R′″, S═O, SO₂, CR″, CR″R′″, SiR″R′″, GeR″R′″, alkyl, cycloalkyl, aryl, heteroaryl, and combinations thereof;
X¹⁰⁰ for each occurrence is selected from the group consisting of O, S, Se, NR″, and CR″R′″;
each R¹⁰, R^{20a}, R^{30a}, R^{40a}, R^{50a}, R^{A″}, R^{B″}, R^{C″}, R^{D″}, R^{E″}, and R^{F″} independently represents mono-, up to the maximum substitutions, or no substitutions;
each R, R′, R″, R′″, R^{10a}, R^{11a}, R^{12a}, R^{13a}, R^{20a}, R^{30a}, R^{40a}, R^{50a}, R⁶⁰, R⁷⁰, R⁹⁷, R⁹⁸, R⁹⁹, R^{A1′}, R^{A2′}, R^{A″}, R^{B″}, R^{C″}, R^{D″}, R^{E″}, R^{F″}, R^{G″}, R^{H″}, R^{I″}, R^{J″}, R^{K″}, R^{L″}, R^{M″}, and R^{N″} is independently a hydrogen or a substituent selected from the group consisting of deuterium, halogen, alkyl, cycloalkyl, heteroalkyl, heterocycloalkyl, boryl, arylalkyl, alkoxy, aryloxy, amino, silyl, germyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof; and
any two adjacent R, R′, R″, R′″, R^{10a}, R^{11a}, R^{12a}, R^{13a}, R^{20a}, R^{30a}, R^{40a}, R^{50a}, R⁶⁰, R⁷⁰, R⁹⁷, R⁹⁸, R⁹⁹, R^{A1′}, R^{A2′}, R^{A″}, R^{B″}, R^{C″}, R^{D″}, R^{E″}, R^{F″}, R^{G″}, R^{H″}, R^{I″}, R^{J″}, R^{K″}, R^{L″}, R^{M″}, and R^{N″} are optionally joined or fused to form a ring.

In some embodiments of LIST 2, each unsubstituted aromatic carbon atom can be replaced with N to form an aza-ring. In some embodiments, the maximum number of N atom in one ring is 1 or 2. In some embodiments of LIST 2, Pt atom in each formula can be replaced by Pd atom.

In some embodiments, compound D2 has a structure selected from the group consisting of the structures of the following LIST 3:

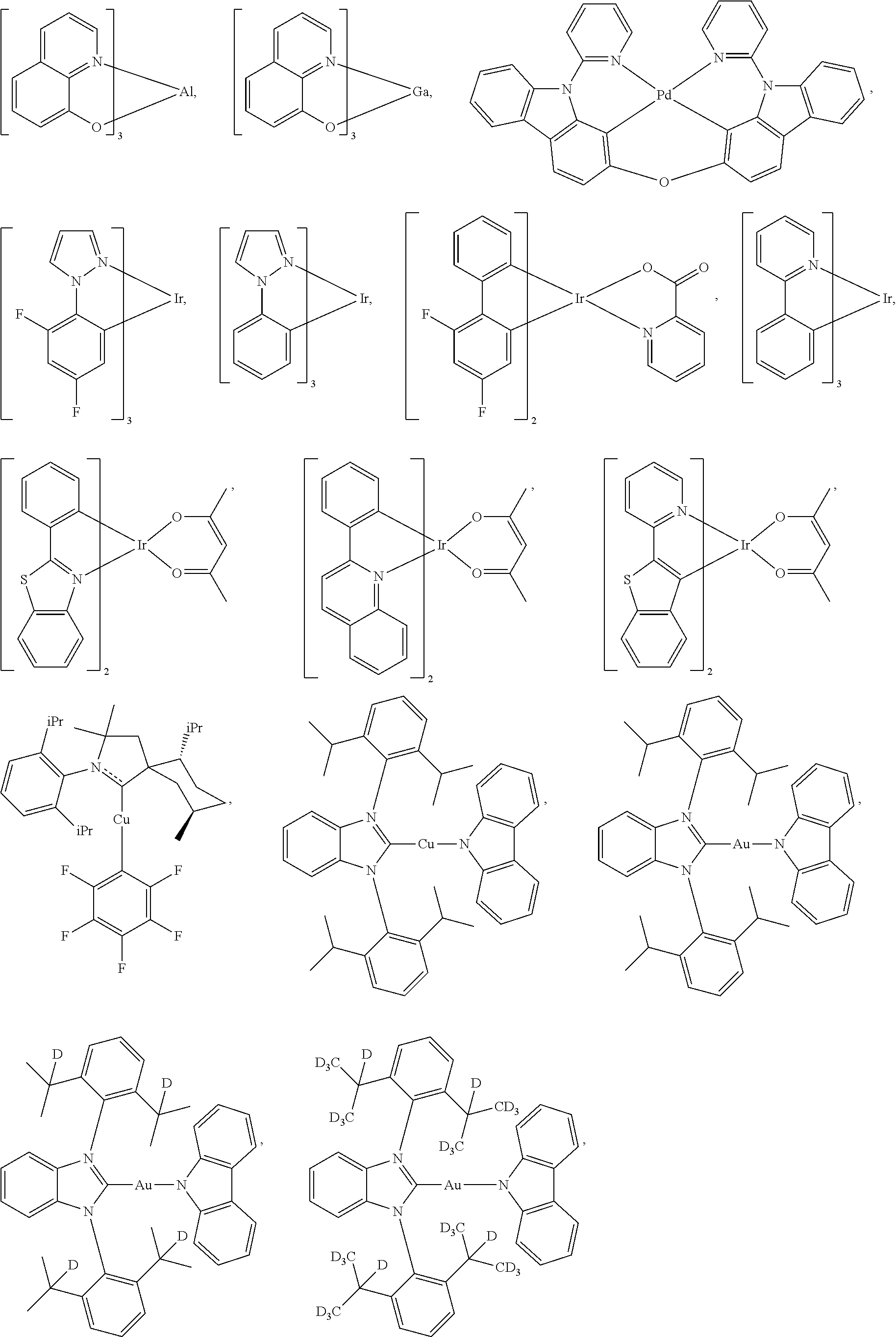

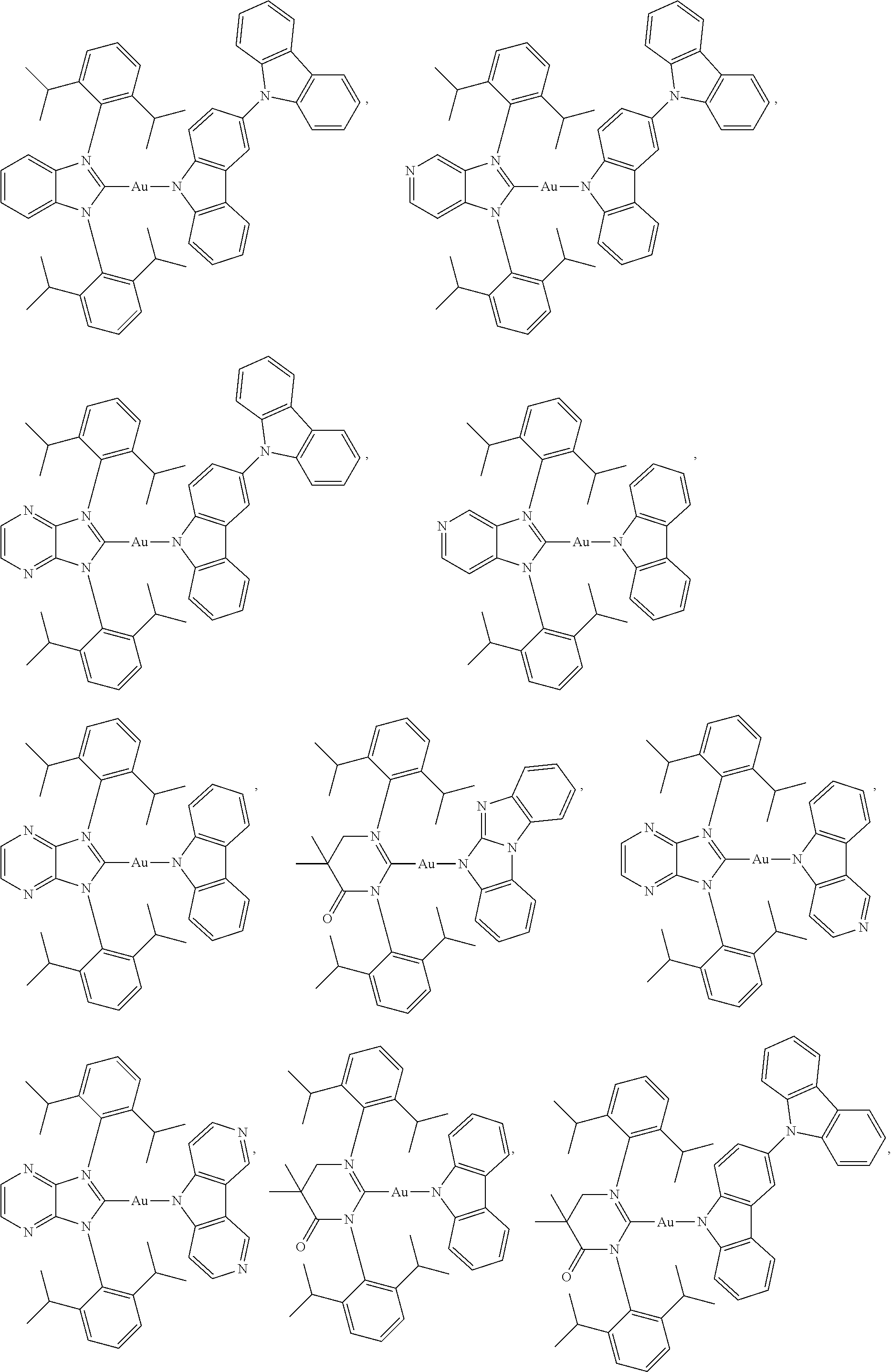

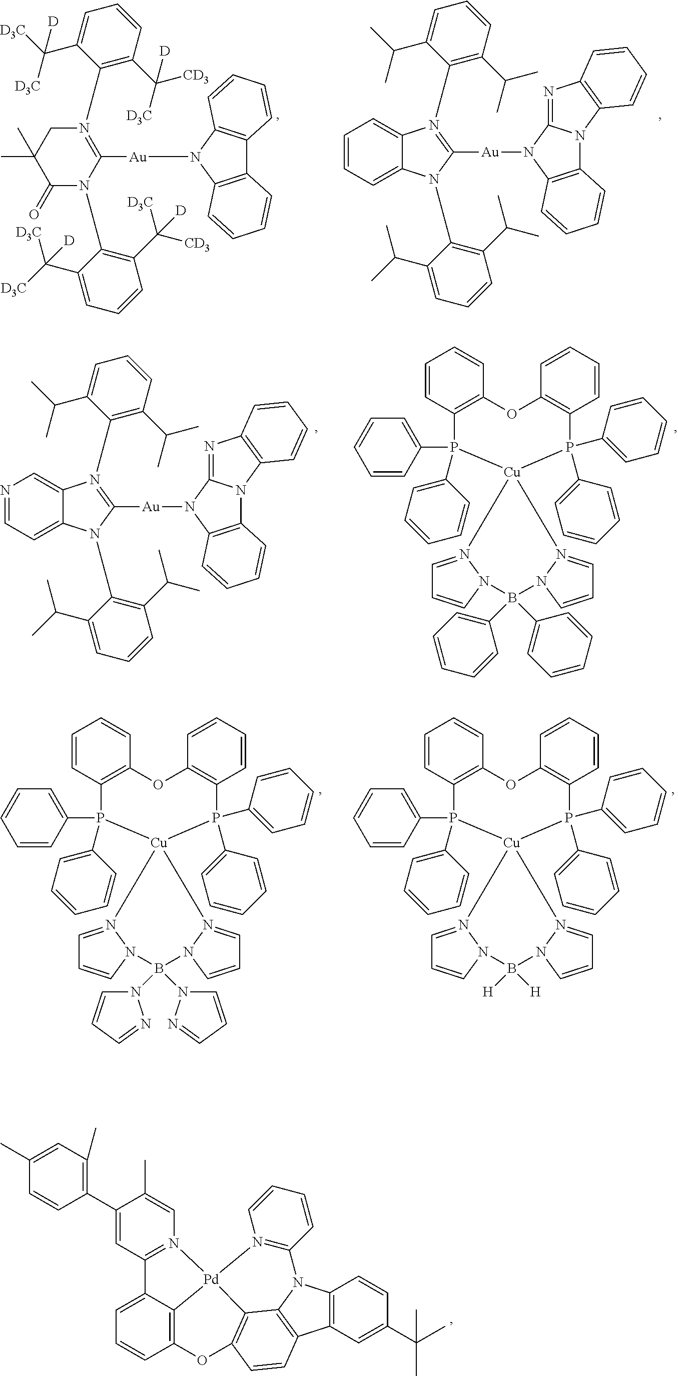

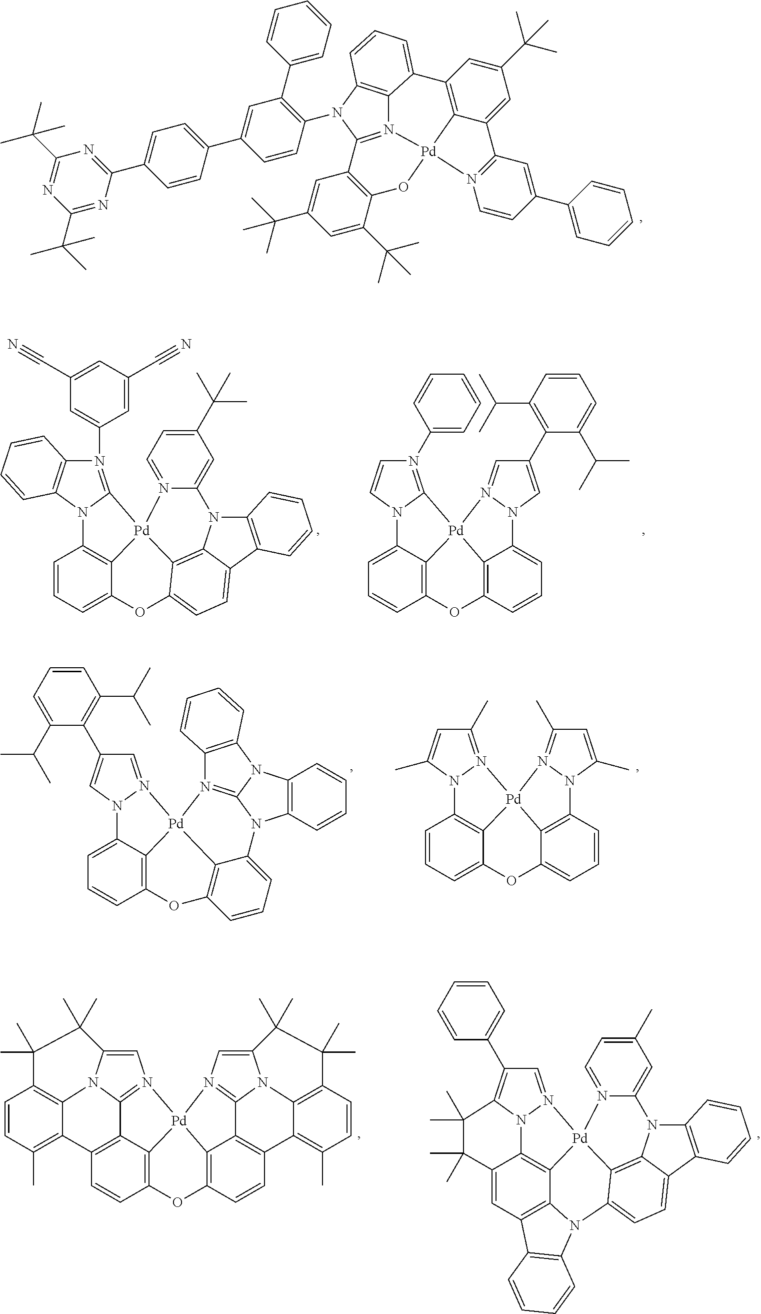

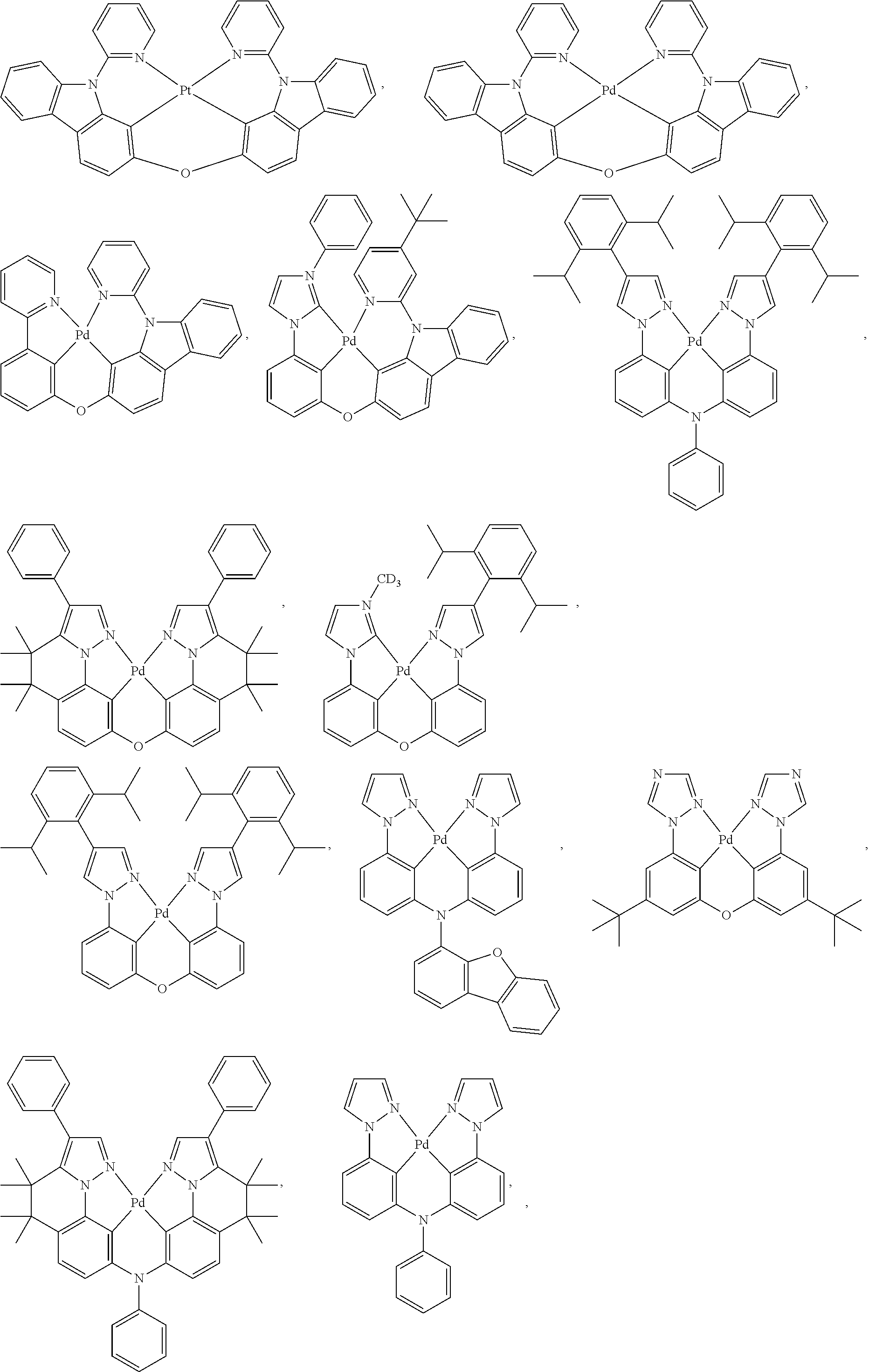

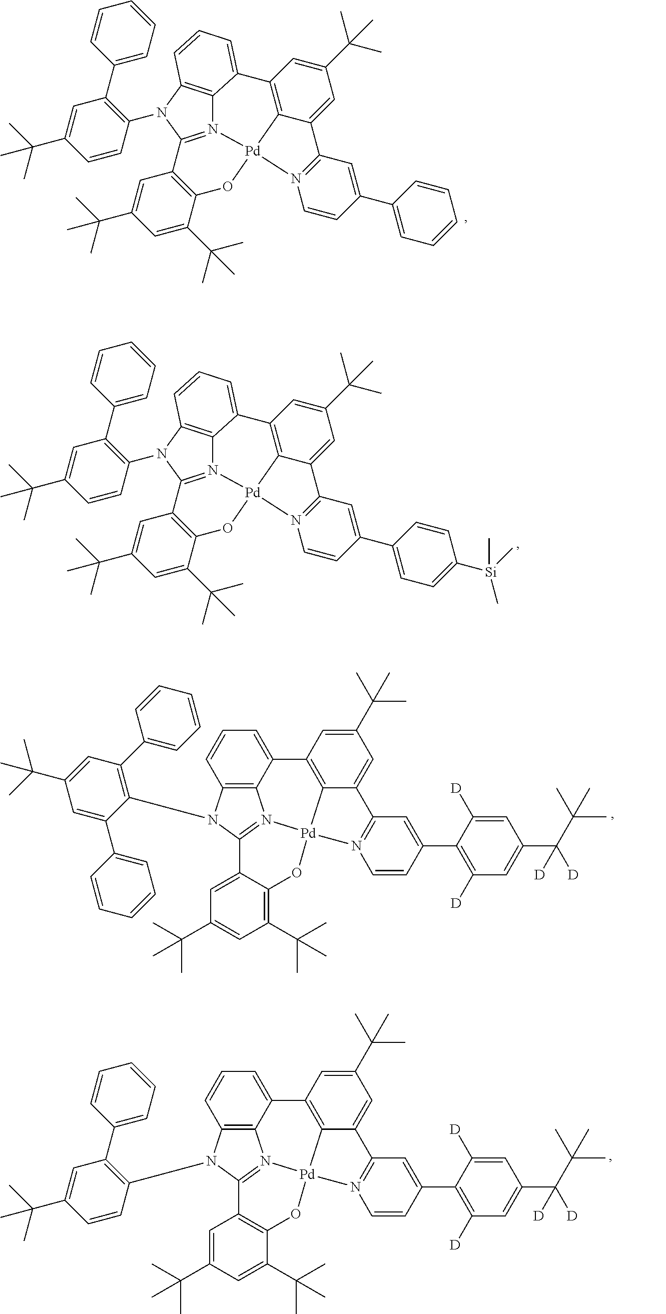

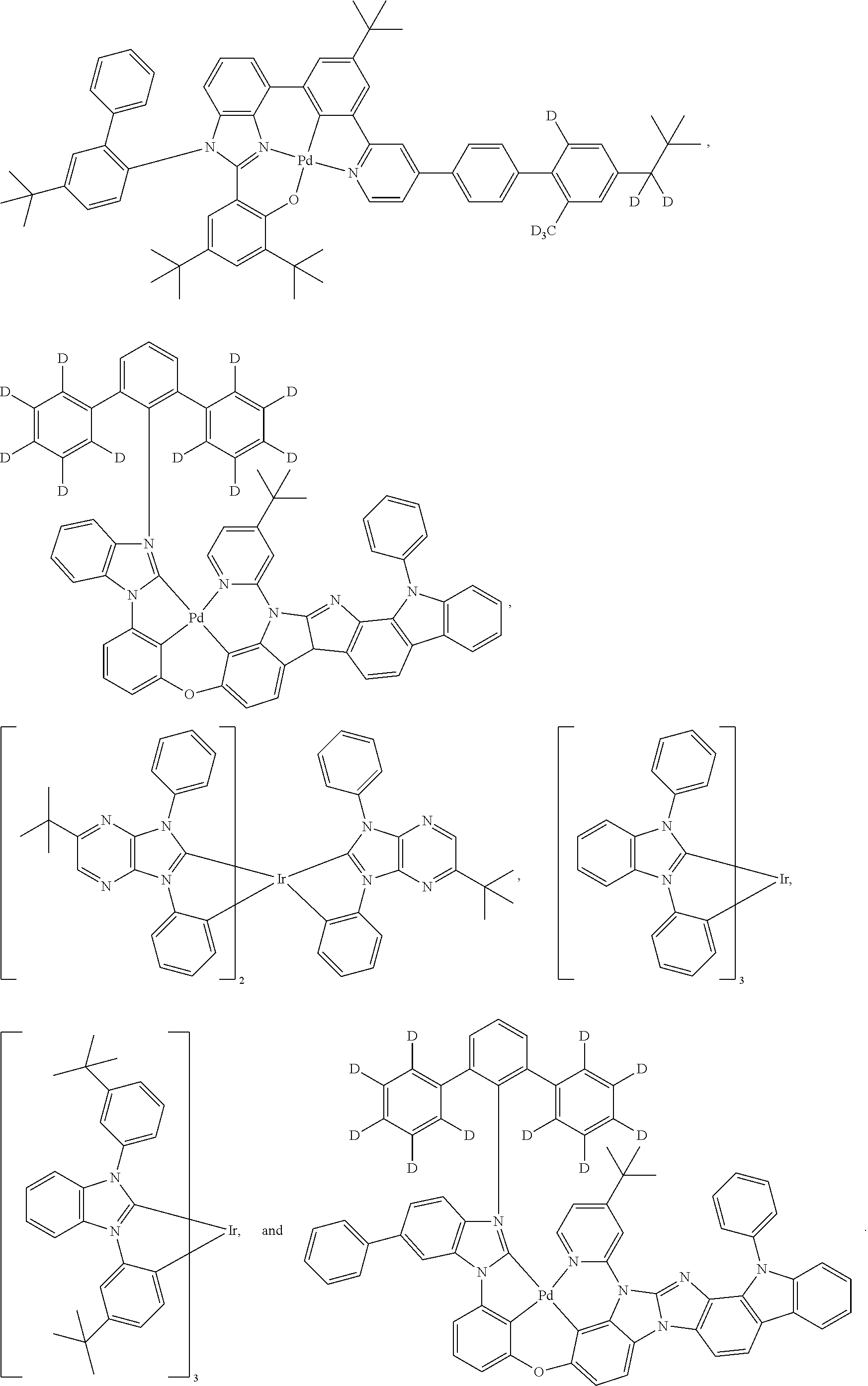

In some such embodiments, D2 is a host material,

In some embodiments of LIST 3, Pd atom in those structures having Pd atom may be replaced by Pt atom.

In some embodiments, the OLED further comprises a first host compound H1.

In some embodiments, the emissive region further comprises the first host compound H1.

In some embodiments, the first host compound H1 comprises at least one chemical moiety selected from the group consisting of triphenylene, carbazole, indolocarbazole, dibenzothiophene, dibenzofuran, dibenzoselenophene, 5λ²-benzo[d]benzo[4,5]imidazo[3,2-a]imidazole, 5,9-dioxa-13b-boranaphtho[3,2,1-de]anthracene, triazine, boryl, silyl, aza-triphenylene, aza-carbazole, aza-indolocarbazole, aza-dibenzothiophene, aza-dibenzofuran, aza-dibenzoselenophene, aza-5λ²-benzo[d]benzo[4,5]imidazo[3,2-a]imidazole, and aza-(5,9-dioxa-13b-boranaphtho[3,2,1-de]anthracene).

In some embodiments, the first host compound H1 is selected from the group consisting of the structures of the following HOST Group 1:

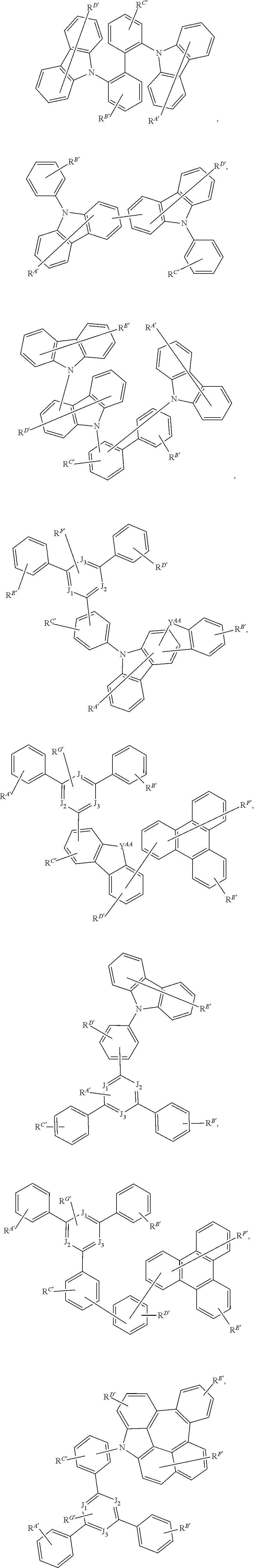

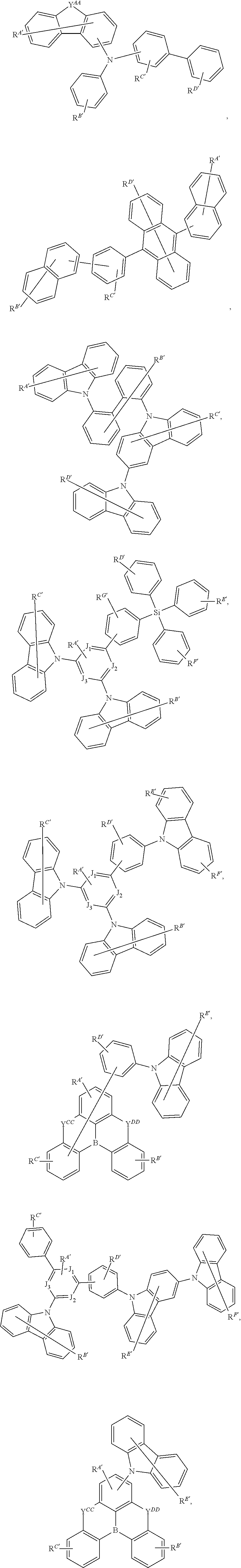

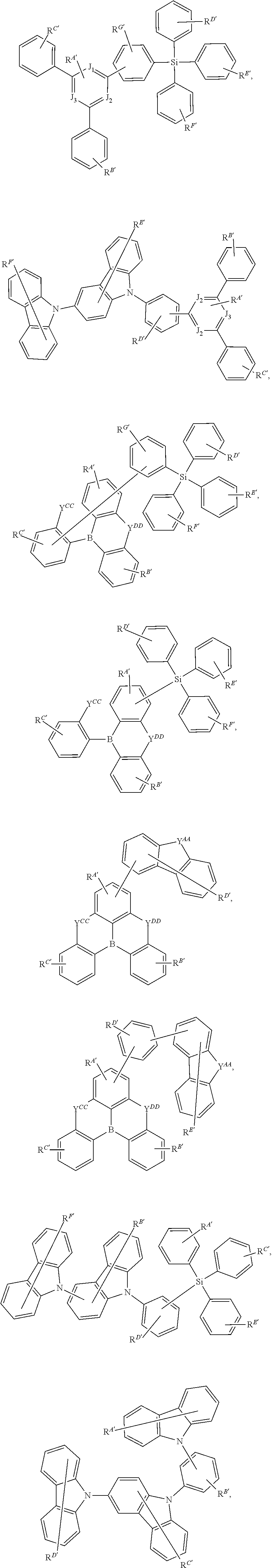

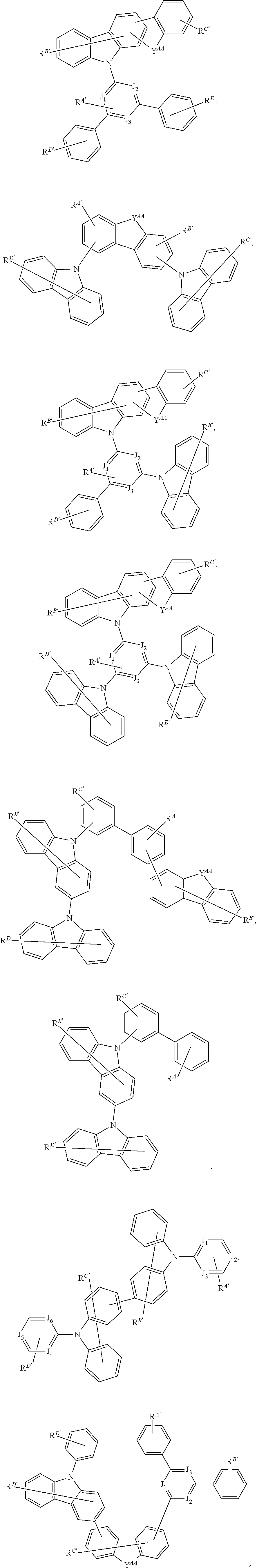

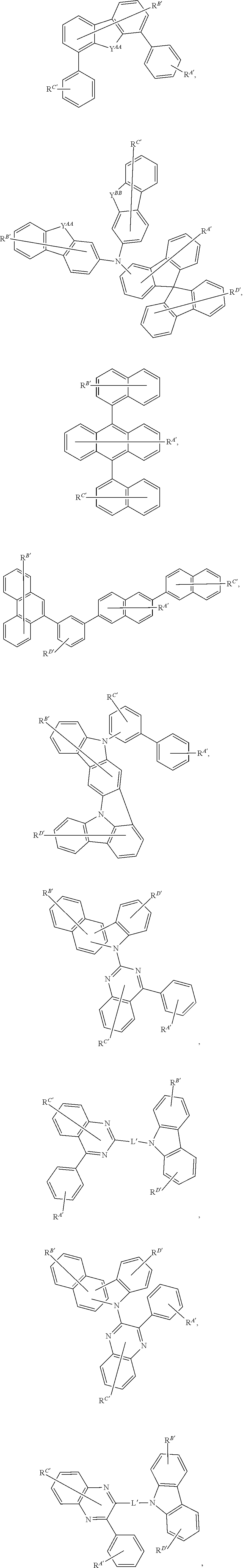

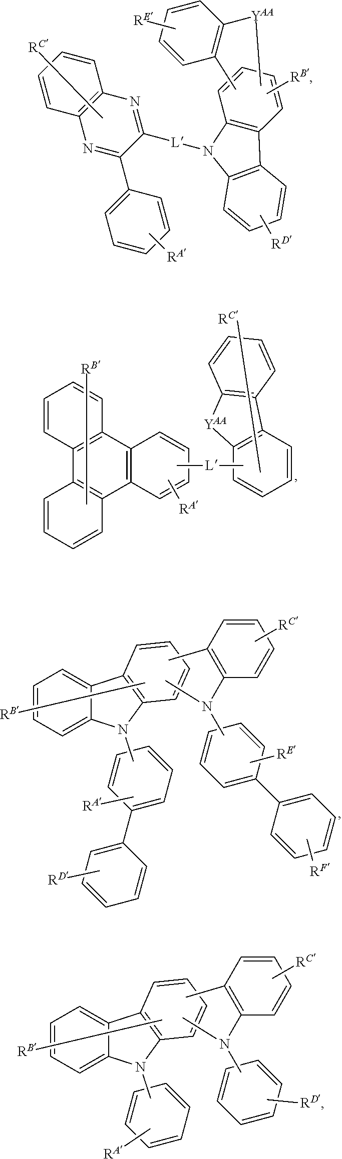

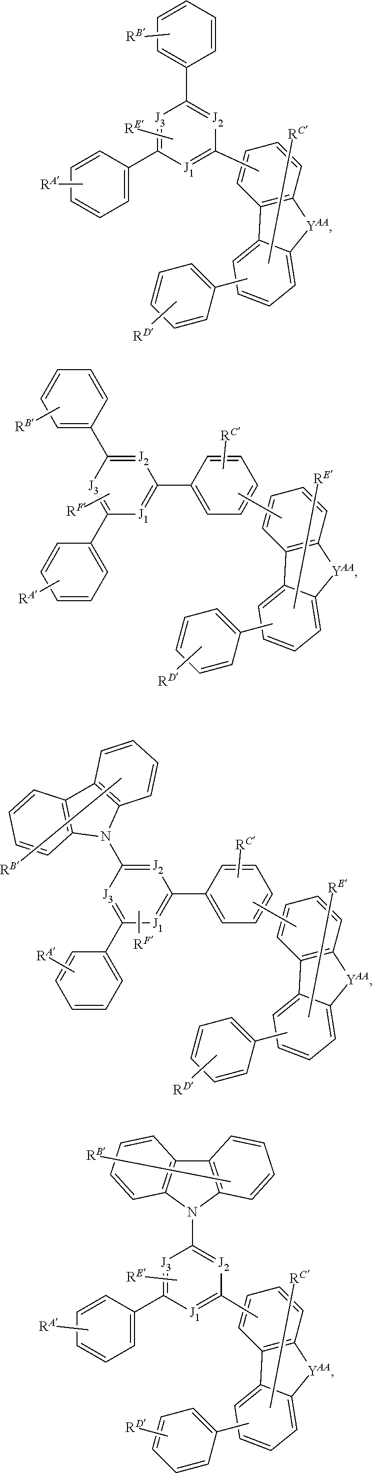

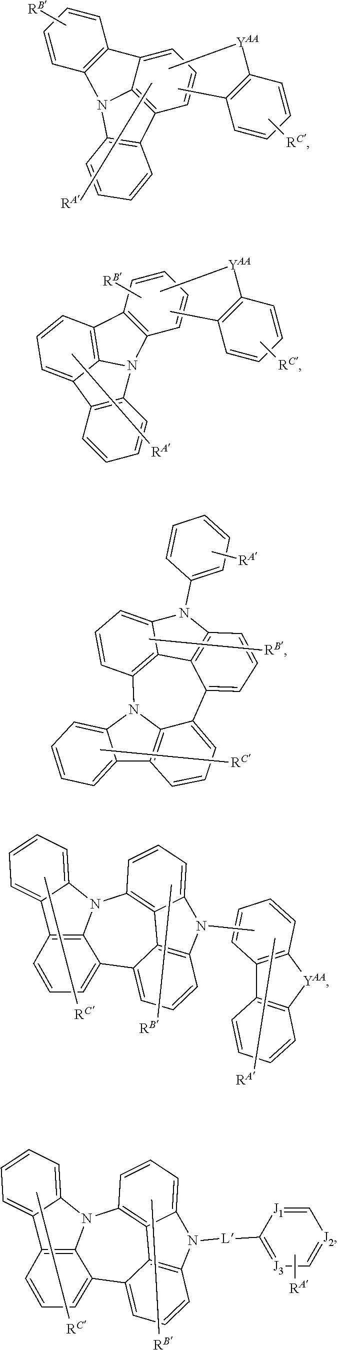

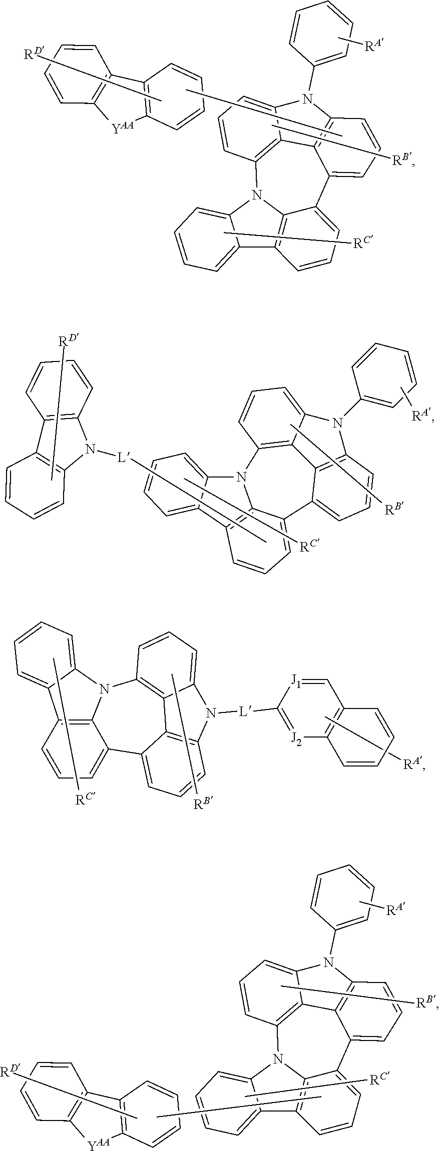

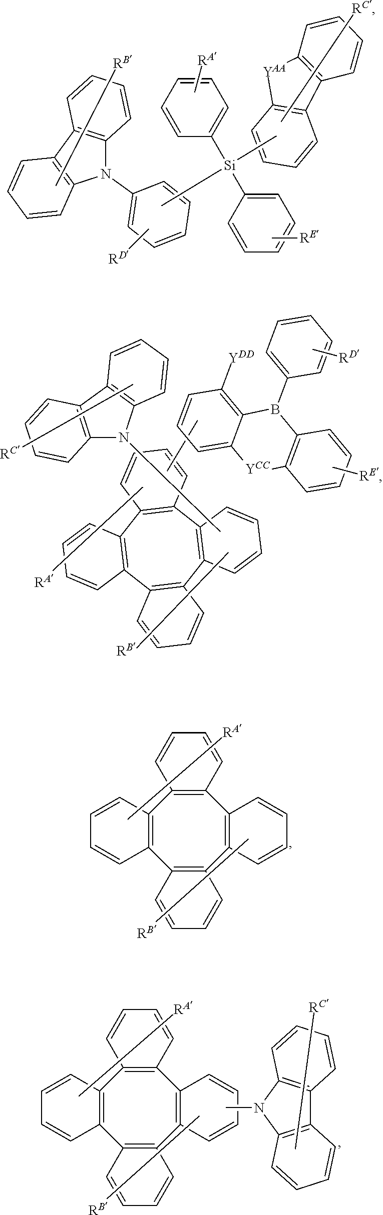

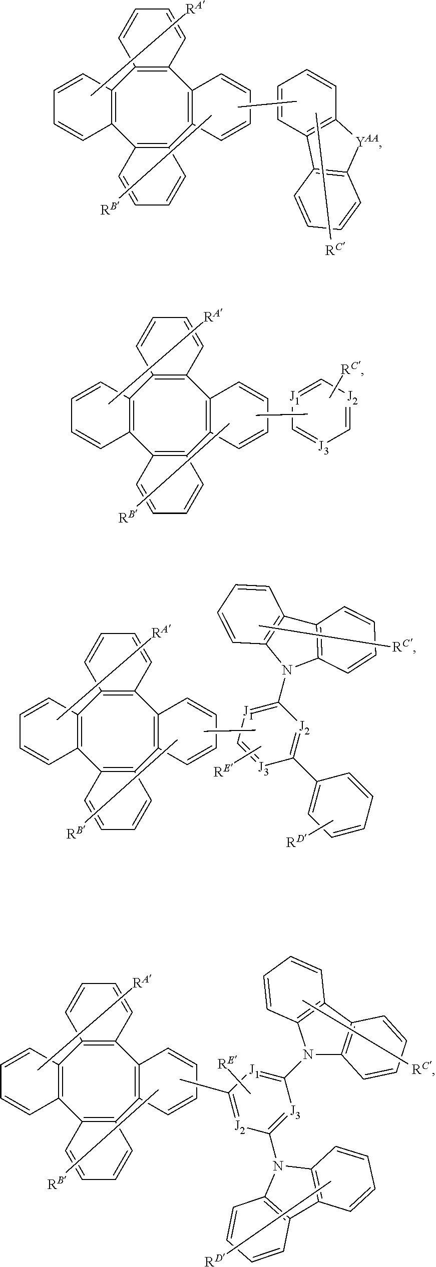

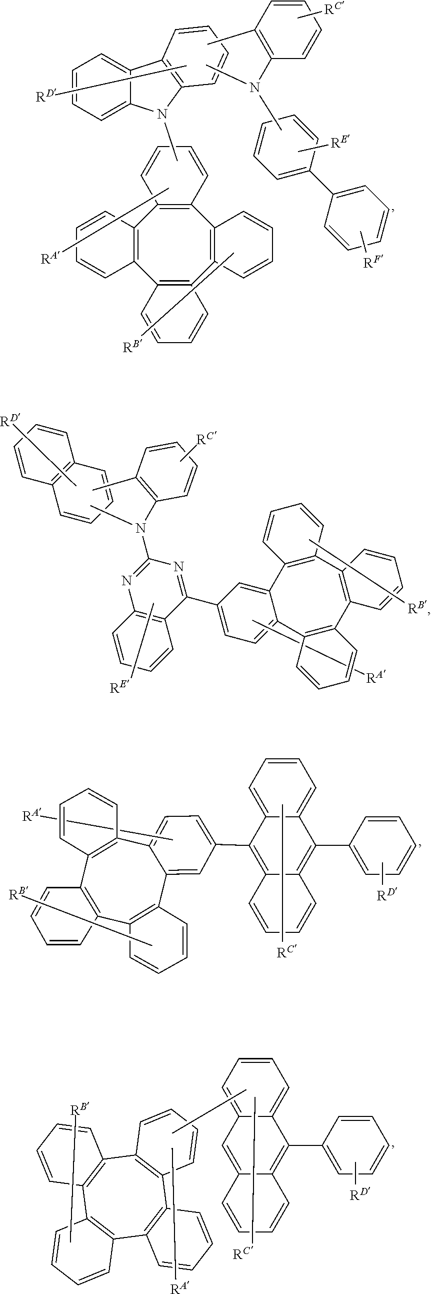

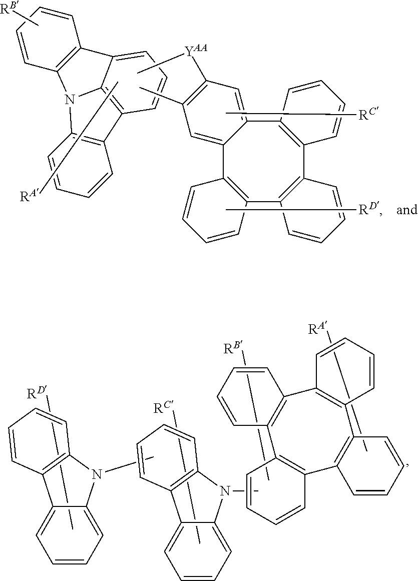

wherein:
each of J₁ to J₆ is independently C or N;
L′ is a direct bond or an organic linker;
each Y^{AA}, Y^{BB}, Y^{CC} and Y^{DD} is independently selected from the group consisting of absent a bond, direct bond, O, S, Se, CRR′, SiRR′, GeRR′, NR, BR, BRR′;
each of R^{A′}, R^{B′}, R^{C′}, R^{D′}, R^{E′}, R^{F′}, and R^{G′} independently represents mono, up to the maximum substitutions, or no substitutions;
each R, R′, R^{A′}, R^{B′}, R^{C′}, R^{D′}, R^{E′}, R^{F′}, and R^{G′} is independently a hydrogen or a substituent selected from the group consisting of the General Substituents as defined herein; any two substituents can be joined or fused to form a ring;
and where possible, each unsubstituted aromatic carbon atom is optionally replaced with N to form an aza-substituted ring.

In some embodiments at least one of J₁ to J₃ is N. In some embodiments at least two of J₁ to J₃ are N. In some embodiments, all three of J₁ to J₃ are N. In some embodiments, each Y^{CC} and Y^{DD} is independently O, S, or SiRR′, or more preferably O or S. In some embodiments, at least one unsubstituted aromatic carbon atom is replaced with N to form an aza-ring.

In some embodiments, H1 may be selected from the group consisting of EG1-MG1-EG1 to EG53-MG27-EG53 with a formula of EGa-MGb-EGc, or EG1-EG1 to EG53-EG53 with a formula of EGa-EGc when MGb is absent, wherein a is an integer from 1 to 53, b is an integer from 1 to 27, c is an integer from 1 to 53. The structure of EG1 to EG53 is shown below:

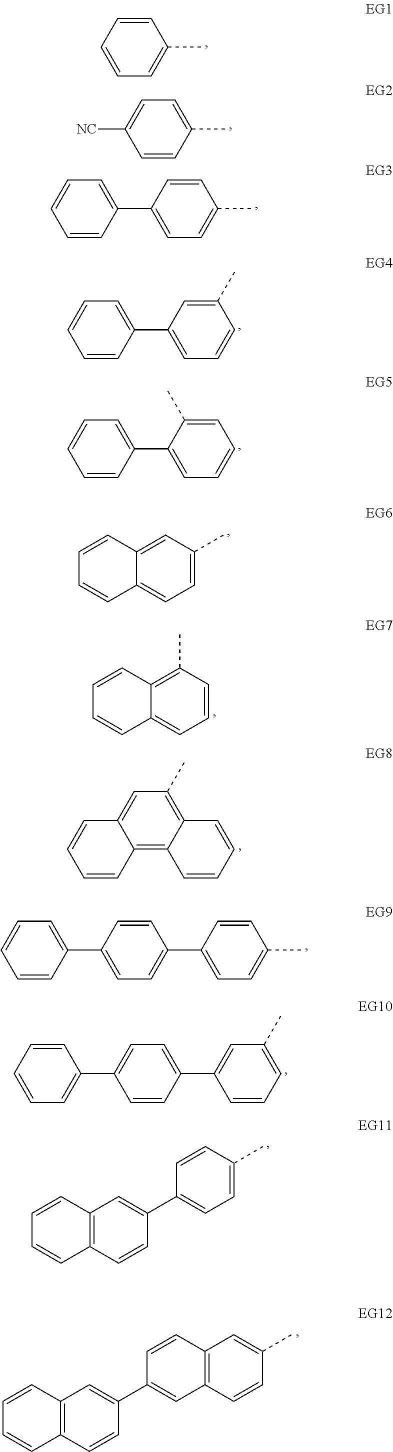

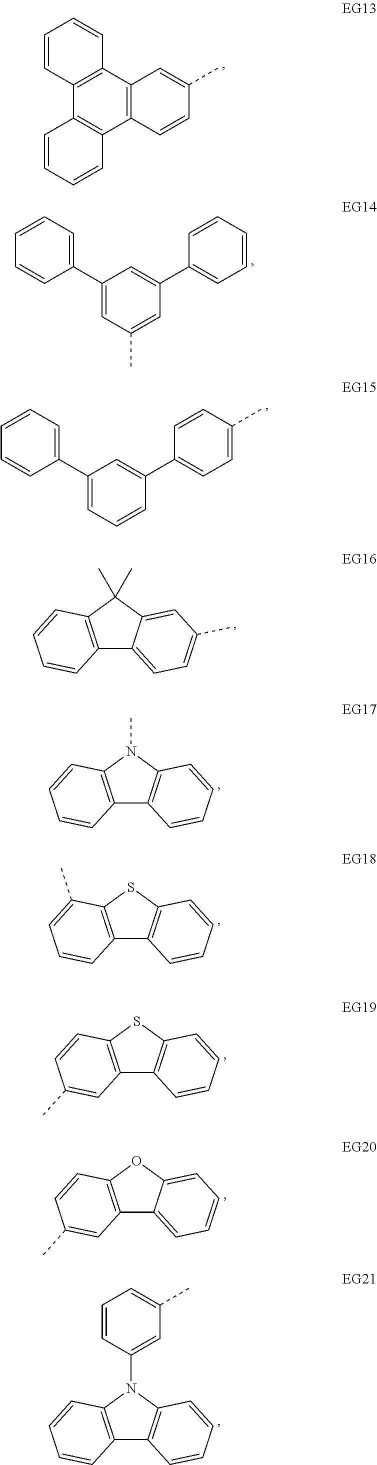

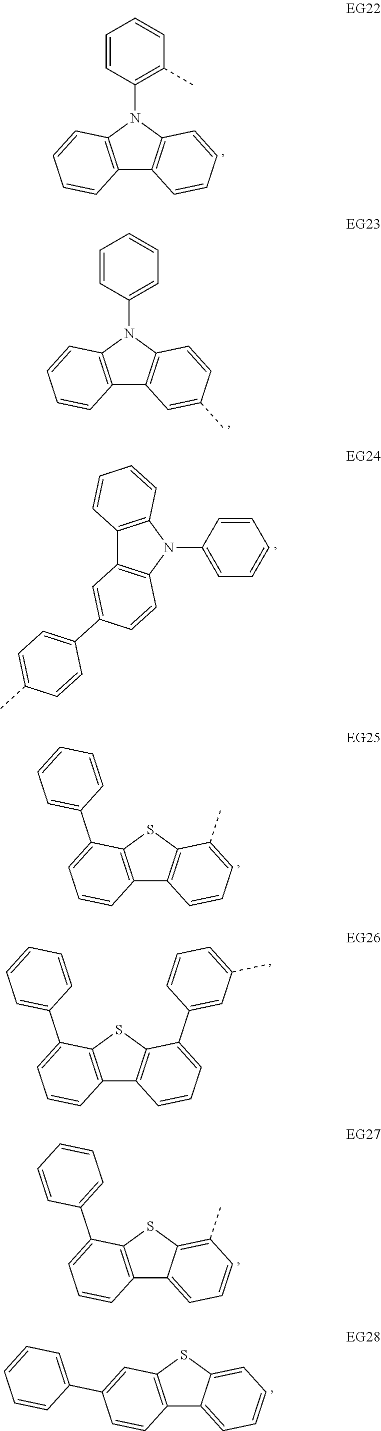

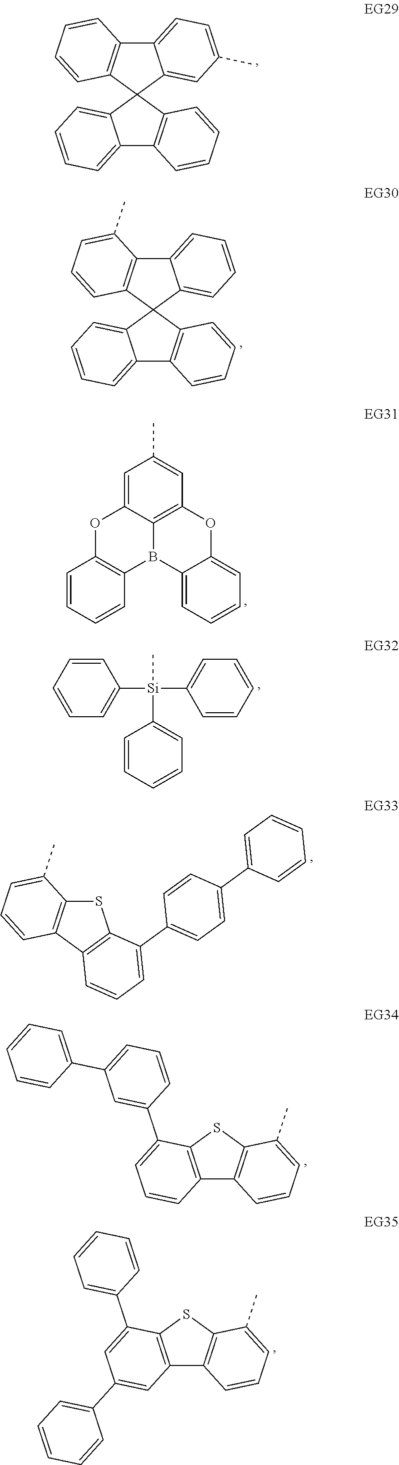

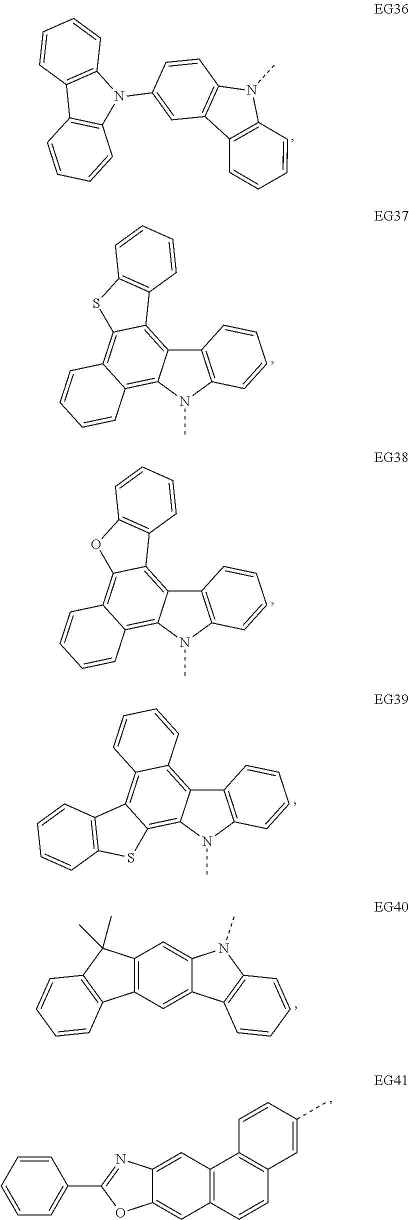

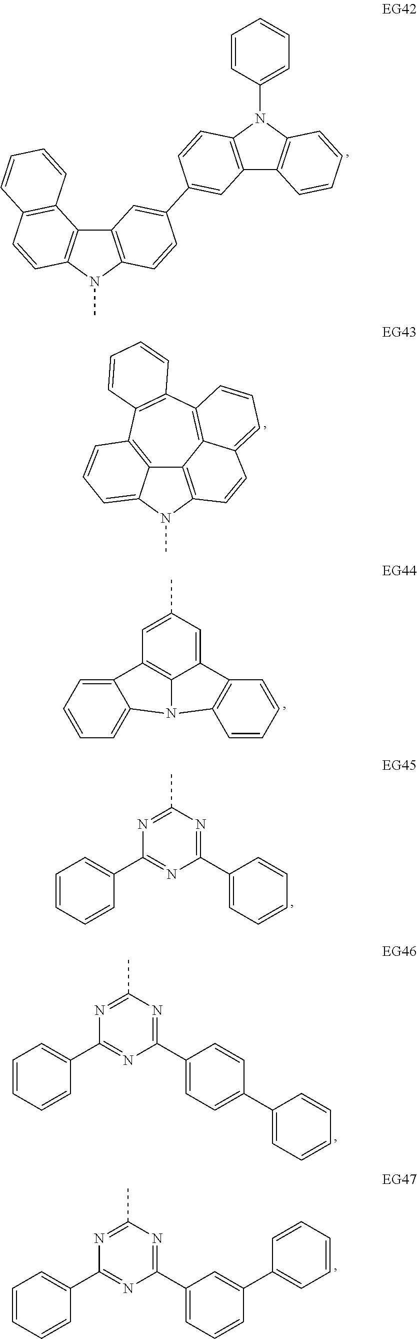

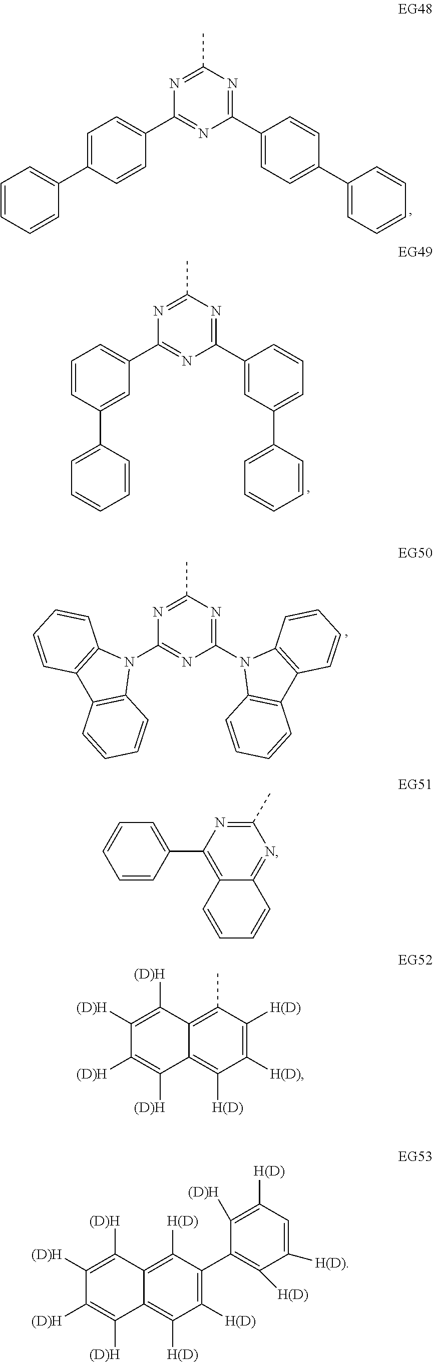

The structure of MG1 to MG27 is shown below:

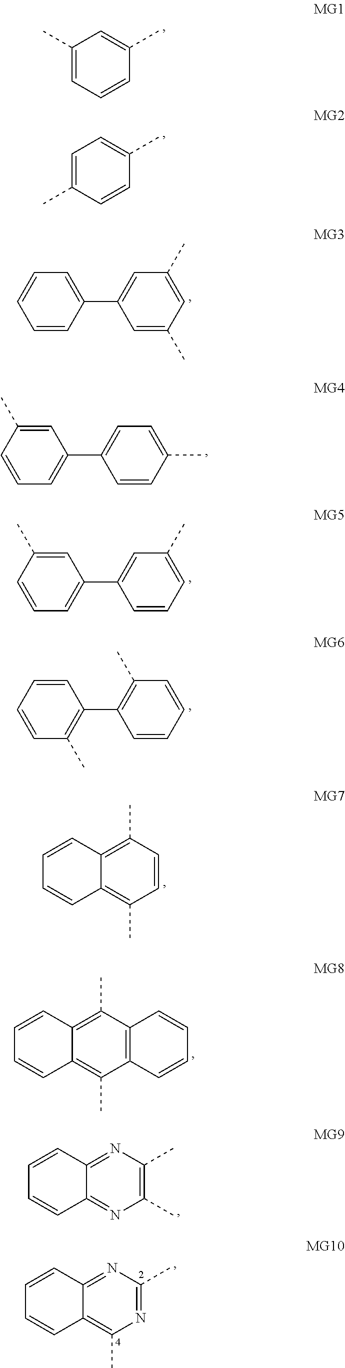

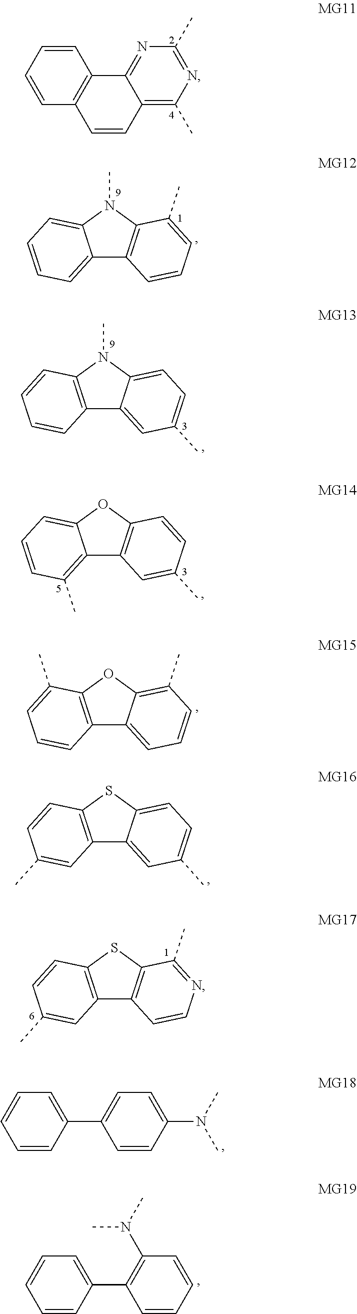

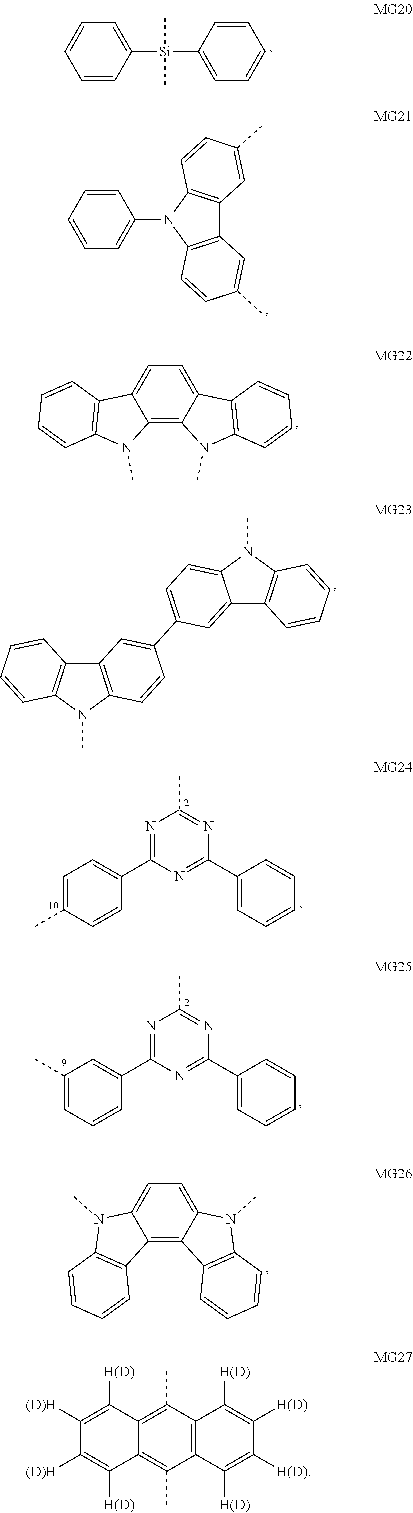

In the MGb structures shown above, the two bonding positions in the asymmetric structures MG10, MG11, MG12, MG13, MG14, MG17, MG24, and MG25 are labeled with numbers for identification purposes.

In some embodiments, H1 may be any of the aza-substituted variants thereof, fully or partially deuterated variants thereof, and combinations thereof. In some embodiments, the host has formula EGa-MGb-Egc and is selected from the group consisting of h1 to h112 defined in the following HOST Group 2 list, where each of MGb, EGa, and EGc are defined as follows:

| h | MGb | EGa | EGc |
|---|---|---|---|
| h1 | MG1 | EG3 | EG36 |
| h2 | MG1 | EG8 | EG12 |
| h3 | MG1 | EG13 | EG14 |
| h4 | MG1 | EG13 | EG18 |
| h5 | MG1 | EG13 | EG25 |
| h6 | MG1 | EG13 | EG36 |
| h7 | MG1 | EG22 | EG36 |
| h8 | MG1 | EG25 | EG46 |
| h9 | MG1 | EG27 | EG46 |
| h10 | MG1 | EG27 | EG48 |
| h11 | MG1 | EG32 | EG50 |
| h12 | MG1 | EG35 | EG46 |
| h13 | MG1 | EG36 | EG45 |
| h14 | MG1 | EG36 | EG49 |
| h15 | MG1 | EG40 | EG45 |
| h16 | MG2 | EG3 | EG36 |
| h17 | MG2 | EG25 | EG31 |
| h18 | MG2 | EG31 | EG33 |
| h19 | MG2 | EG36 | EG45 |
| h20 | MG2 | EG36 | EG46 |
| h21 | MG3 | EG4 | EG36 |
| h22 | MG3 | EG34 | EG45 |
| h23 | MG4 | EG13 | EG17 |
| h24 | MG5 | EG13 | EG45 |
| h25 | MG5 | EG17 | EG36 |
| h26 | MG5 | EG18 | EG36 |
| h27 | MG6 | EG17 | EG17 |
| h28 | MG7 | EG43 | EG45 |
| h29 | MG8 | EG1 | EG28 |
| h30 | MG8 | EG6 | EG7 |
| h31 | MG8 | EG7 | EG7 |
| h32 | MG8 | EG7 | EG11 |
| h33 | MG9 | EG1 | EG43 |
| h34 | MG10 | 4-EG1 | 2-EG37 |
| h35 | MG10 | 4-EG1 | 2-EG38 |
| h36 | MG10 | EG1 | EG42 |
| h37 | MG11 | 4-EG1 | 2-EG39 |
| h38 | MG12 | 1-EG17 | 9-EG31 |
| h39 | MG13 | 3-EG17 | 9-EG4 |
| h40 | MG13 | 3-EG17 | 9-EG13 |
| h41 | MG13 | 3-EG17 | 9-EG31 |
| h42 | MG13 | 3-EG17 | 9-EG45 |
| h43 | MG13 | 3-EG17 | 9-EG46 |
| h44 | MG13 | 3-EG17 | 9-EG48 |
| h45 | MG13 | 3-EG17 | 9-EG49 |
| h46 | MG13 | 3-EG32 | 9-EG31 |
| h47 | MG13 | 3-EG44 | 9-EG3 |
| h48 | MG14 | 3-EG13 | 5-EG45 |
| h49 | MG14 | 3-EG23 | 5-EG45 |
| h50 | MG15 | EG3 | EG48 |
| h51 | MG15 | EG17 | EG31 |
| h52 | MG15 | EG31 | EG36 |
| h53 | MG16 | EG17 | EG17 |
| h54 | MG17 | EG17 | EG17 |
| h55 | MG18 | EG16 | EG24 |
| h56 | MG18 | EG16 | EG30 |
| h57 | MG18 | EG20 | EG41 |
| h58 | MG19 | EG16 | EG29 |
| h59 | MG20 | EG1 | EG31 |
| h60 | MG20 | EG17 | EG18 |
| h61 | MG21 | EG23 | EG23 |
| h62 | MG22 | EG1 | EG45 |
| h63 | MG22 | EG1 | EG46 |
| h64 | MG22 | EG3 | EG46 |
| h65 | MG22 | EG4 | EG46 |
| h66 | MG22 | EG4 | EG47 |
| h67 | MG22 | EG9 | EG45 |
| h68 | MG23 | EG1 | EG3 |
| h69 | MG23 | EG1 | EG6 |
| h70 | MG23 | EG1 | EG14 |
| h71 | MG23 | EG1 | EG18 |
| h72 | MG23 | EG1 | EG19 |
| h73 | MG23 | EG1 | EG23 |
| h74 | MG23 | EG1 | EG51 |
| h75 | MG23 | EG2 | EG18 |
| h76 | MG23 | EG3 | EG3 |
| h77 | MG23 | EG3 | EG4 |
| h78 | MG23 | EG3 | EG5 |
| h79 | MG23 | EG4 | EG4 |
| h80 | MG23 | EG4 | EG5 |
| h81 | MG24 | 2-EG1 | 10-EG33 |
| h82 | MG24 | 2-EG4 | 10-EG36 |
| h83 | MG24 | 2-EG21 | 10-EG36 |
| h84 | MG24 | 2-EG23 | 10-EG36 |
| h85 | MG25 | 2-EG1 | 9-EG33 |
| h86 | MG25 | 2-EG3 | 9-EG36 |
| h87 | MG25 | 2-EG4 | 9-EG36 |
| h88 | MG25 | 2-EG17 | 9-EG27 |
| h89 | MG25 | 2-EG17 | 9-EG36 |
| h90 | MG25 | 2-EG21 | 9-EG36 |
| h91 | MG25 | 2-EG23 | 9-EG27 |
| h92 | MG25 | 2-EG23 | 9-EG36 |
| h93 | MG26 | EG1 | EG9 |
| h94 | MG26 | EG1 | EG10 |
| h95 | MG26 | EG1 | EG21 |
| h96 | MG26 | EG1 | EG23 |
| h97 | MG26 | EG1 | EG26 |
| h98 | MG26 | EG3 | EG3 |
| h99 | MG26 | EG3 | EG9 |
| h100 | MG26 | EG3 | EG23 |
| h101 | MG26 | EG3 | EG26 |
| h102 | MG26 | EG4 | EG10 |
| h103 | MG26 | EG5 | EG10 |
| h104 | MG26 | EG6 | EG10 |
| h105 | MG26 | EG10 | EG10 |
| h106 | MG26 | EG10 | EG14 |
| h107 | MG26 | EG10 | EG15 |
| h108 | MG27 | EG52 | EG53 |
| h109 | — | EG13 | EG18 |
| h110 | — | EG17 | EG31 |
| h111 | — | EG17 | EG50 |
| h112 | — | EG40 | EG45 |

In the table above, the EGa and EGc structures that are bonded to one of the asymmetric structures MG10, MG11, MG12, MG13, MG14, MG17, MG24, and MG25, are noted with a numeric prefix identifying their bonding position in the MGb structure.

In some embodiments, the first host compound H1 is selected from the group consisting of the structures of the structures of the following HOST Group 3:

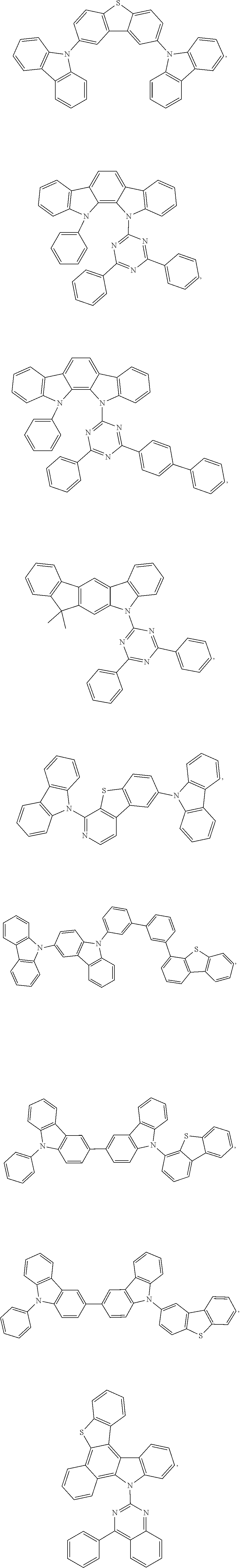

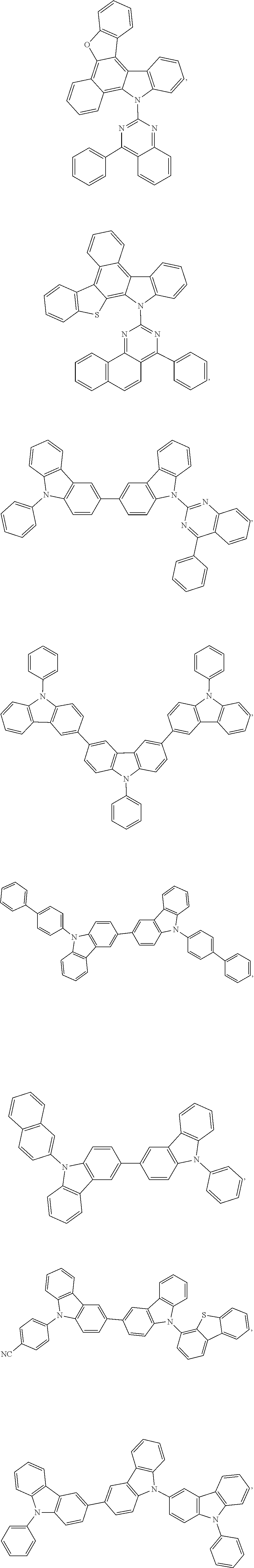

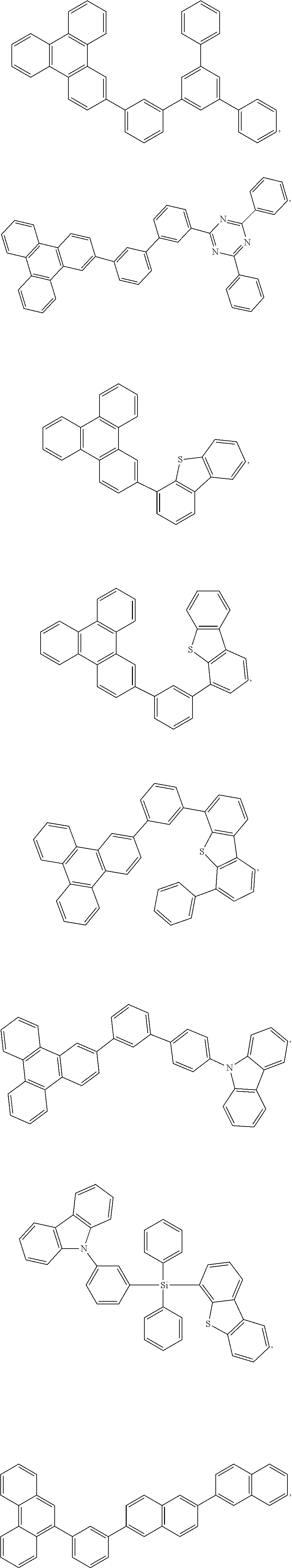

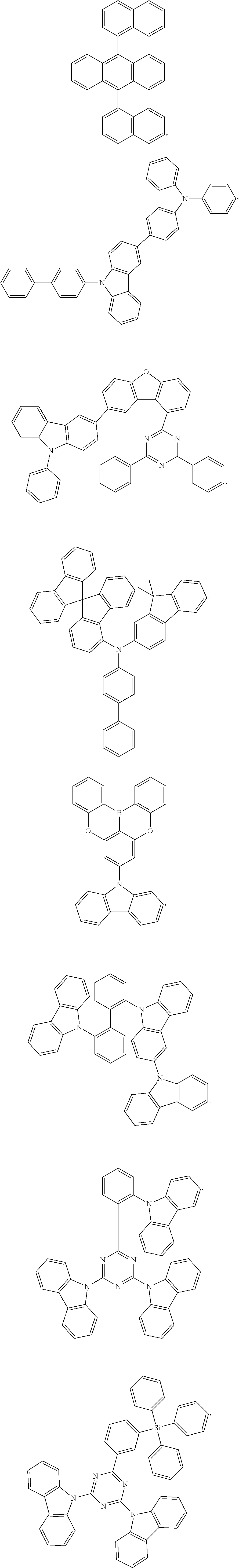

and combinations thereof.

In some embodiments, the OLED further comprises a second host compound H2.

In some embodiments, the emissive region further comprises the second host compound H2.

In some embodiments, the second host compound H2 comprises at least one chemical moiety selected from the group consisting of triphenylene, carbazole, indolocarbazole, dibenzothiophene, dibenzofuran, dibenzoselenophene, 5λ²-benzo[d]benzo[4,5]imidazo[3,2-a]imidazole, 5,9-dioxa-13b-boranaphtho[3,2,1-de]anthracene, triazine, boryl, silyl, aza-triphenylene, aza-carbazole, aza-indolocarbazole, aza-dibenzothiophene, aza-dibenzofuran, aza-dibenzoselenophene, aza-5λ²-benzo[d]benzo[4,5]imidazo[3,2-a]imidazole, and aza-(5,9-dioxa-13b-boranaphtho[3,2,1-de]anthracene).

In some embodiments, the second host compound H2 is selected from the group consisting of the structures of HOST Group 1 defined herein. In some embodiments, the second host compound H2 is selected from the group consisting of the structures of HOST Group 2 defined herein.

In some embodiments, the OLED emits a luminescent radiation at room temperature when a voltage is applied across the device; wherein the luminescent radiation comprises a first radiation component contributed from the first compound D1 with an emission λₘₐₓ of 380-480 nm.

In some embodiments, the OLED emits a luminescent radiation at room temperature when a voltage is applied across the device; wherein the luminescent radiation comprises a first radiation component contributed from the first compound D1 with an emission λₘₐₓ of 480-550 nm.

In some embodiments, the OLED emits a luminescent radiation at room temperature when a voltage is applied across the device; wherein the luminescent radiation comprises a first radiation component contributed from the first compound D1 with an emission max of 550-700 nm.

In some embodiments, the second compound D2 comprises a metal M2 selected from the group consisting of Pt, Pd, Au, Ag, Cu, Zn, Zr, Ru, Re, Co, Ti, Os, Ga, Al, Rh, Mn, Mg, Ni, Fe, Tl, Pb, Po, At, Sb, Te, In, Bi, and Sn. In some embodiments, the metal M2 is selected from the group consisting of Pt, Pd, Au, Ag, Cu, Zn, and Ni.

In some embodiments, the OLED emits a luminescent radiation at room temperature when a voltage is applied across the device; wherein the luminescent radiation comprises a first radiation component contributed from the first compound D1 with an emission max of 700-1000 nm.

In some embodiments, the second compound D2 comprises a metal M2 selected from the group consisting of Ir, Pt, Pt, Pd, Au, Al, Ag, Cu, Zn, and Ni.

In some embodiments, a concentration of D1 in the emissive region is less than a concentration of D2 in the emissive region.

In some embodiments, each of D1 and D2 is present in the emissive region in a concentration greater than 3% by volume in the emissive region. In some embodiments, each of D1 and D2 is present in the emissive region in a concentration greater than 5% by volume in the emissive region. In some embodiments, each of D1 and D2 is present in the emissive region in a concentration greater than 8% by volume in the emissive region. In some embodiments, each of D1 and D2 is present in the emissive region in a concentration greater than 10% by volume in the emissive region.

In some embodiments, D2 serves as a host for D1 in the emissive region. In some such embodiments, a HOMO level of D1, E_{HOMO,D1}, is lower than a HOMO level of D2, E_{HOMO,D2}.

In some embodiments, a concentration of D2 in the emissive region is less than a concentration of the D1 in the emissive region. In some embodiments, the concentration of D2 in the emissive region is less than 5% by volume. In some embodiments, the concentration of D2 in the emissive region is less than 3% by volume. In some embodiments, the concentration of D2 in the emissive region is less than 2% by volume. In some embodiments, the concentration of D2 in the emissive region is less than 1% by volume. In some embodiments, the concentration of D2 in the emissive region is less than 0.1% by volume.

In some embodiments, D1 and D2 are deposited to form the emissive region using a pre-mixed source.

In some embodiments, a triplet energy of D2, E_{T,D2}, is greater than a triplet energy of D1, E_{T,D1}.

In some embodiments, the emissive region comprises at least one material with a HOMO level higher than a HOMO level of D2, E_{HOMO,D2}.

In some embodiments, a HOMO level of D2, E_{HOMO,D2}, is lower than a HOMO level of D1, E_{HOMO,D1}.

In some embodiments, the emissive region comprises a first host material, H1 and a second host material, H2, and a HOMO level of D2, E_{HOMO,D2}, is lower than at least one of a HOMO level of H1, E_{HOMO,H1}, and a HOMO level of H2, E_{HOMO,H2}.

In some embodiments, a HOMO level of D2, E_{HOMO,D2}, is the lowest HOMO level of any material in the emissive region.

In some embodiments, the emissive region comprises at least one material with a LUMO level lower than a LUMO level of D2, E_{LUMO,D2}.

In some embodiments, a LUMO level of D2, E_{LUMO,D2}, is higher than a LUMO level of D1, E_{LUMO,D1}.

In some embodiments, the emissive region comprises a first host material, H1 and a second host material, H2, and wherein a LUMO level of D2, E_{LUMO,D2}, is higher than at least one of a LUMO level of H1, E_{LUMO,H1}, and a LUMO level of H2, E_{LUMO,H2}.

In some embodiments, a LUMO level of D2, E_{LUMO,D2}, is the highest LUMO level of any materials in the emissive region.

In some embodiments, the compound D1 is a metal coordination complex having a metal-carbon bond.

In some embodiments, the compound D1 is a metal coordination complex having a metal-nitrogen bond.

In some embodiments, the compound D1 is a metal coordination complex having a metal-oxygen bond.

In some embodiments, the compound D1 comprises a metal M1 selected from the group consisting of Ir, Rh, Re, Ru, Os, Pt, Pd, Au, and Cu. In some embodiments, M1 is Ir. In some embodiments, M1 is Pt.

In some embodiments, the compound D1 has the formula of M(L¹)ₓ(L²)_{y}(L³)_{z}; wherein L¹, L², and L³ can be the same or different;
wherein x is 1, 2, or 3;
wherein y is 0, 1, or 2;
wherein z is 0, 1, or 2;
wherein x+y+z is the oxidation state of the metal M;
wherein L¹ is selected from the group consisting of the structures of LIST 1 defined herein;
wherein L² and L³ are independently selected from the group consisting of

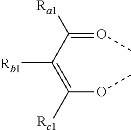

and the structures of LIST 1 defined herein;
   wherein:
T is selected from the group consisting of B, Al, Ga, and In;
K^{1′} is a direct bond or is selected from the group consisting of NRₑ, PRₑ, O, S, and Se;
each Y¹ to Y¹³ are independently selected from the group consisting of carbon and nitrogen;
Y′ is selected from the group consisting of BRₑ, NRₑ, P Rₑ, O, S, Se, C═O, S═O, SO₂, CRₑR_{f}, SiRₑR_{f}, and GeRₑR_{f};
Rₑ and R_{f} can be fused or joined to form a ring;
each Rₐ, R_{b}, R_{c}, and R_{d} can independently represent from mono to the maximum possible number of substitutions, or no substitution;
each Rₐ₁, R_{b1}, R_{c1}, R_{d1}, Rₐ, R_{b}, R_{c}, R_{d}, Rₑ, and R_{f} is independently a hydrogen or a substituent selected from the group consisting of deuterium, halogen, alkyl, cycloalkyl, heteroalkyl, heterocycloalkyl, boryl, arylalkyl, alkoxy, aryloxy, amino, silyl, germyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof; and
any two adjacent substituents of Rₐ₁, R_{b1}, R_{c1}, R_{d1}, Rₐ, R_{b}, R_{c}, and R_{d} can be fused or joined to form a ring or form a multidentate ligand.

In some embodiments, D1 is selected from the group consisting of the structures of LIST 2 defined herein;

wherein:
each of X⁹⁶ to X⁹⁹ is independently C or N;
each of Y¹⁰⁰ and Y²⁰⁰ is independently selected from the group consisting of a NR″, O, S, and Se;
L is independently selected from the group consisting of a direct bond, BR″, BR″R′″, NR″, PR″, O, S, Se, C═O, C═S, C═Se, C═NR″, C═CR″R′″, S═O, SO₂, CR″, CR″R′″, SiR″R′″, GeR″R′″, alkyl, cycloalkyl, aryl, heteroaryl, and combinations thereof;
X¹⁰⁰ for each occurrence is selected from the group consisting of O, S, Se, NR″, and CR″R′″;
each R^{10a}, R^{20a}, R^{30a}, R^{40a}, R^{50a}, R^{A″}, R^{B″}, R^{C″}, R^{D″}, R^{E″}, and R^{F}″ independently represents mono-, up to the maximum substitutions, or no substitutions;
each R, R′, R″, R′″, R^{10a}, R^{11a}, R^{12a}, R^{13a}, R^{20a}, R^{30a}, R^{40a}, R^{50a}, R⁶⁰, R⁷⁰, R⁹⁷, R⁹⁸, R⁹⁹, R^{A1′}, R^{A2′}, R^{A″}, R^{B″}, R^{C″}, R^{D″}, R^{E″}, R^{F″}, R^{G″}, R^{H″}, R^{I″}, R^{J″}, R^{K″}, R^{L″}, R^{M″}, and R^{N}″ is independently a hydrogen or a substituent selected from the group consisting of deuterium, halogen, alkyl, cycloalkyl, heteroalkyl, heterocycloalkyl, boryl, arylalkyl, alkoxy, aryloxy, amino, silyl, germyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof; and any two substituents can be joined or fused into a ring.

In some embodiments, the compound D1 is selected from the group consisting of the structures of the following LIST 4:

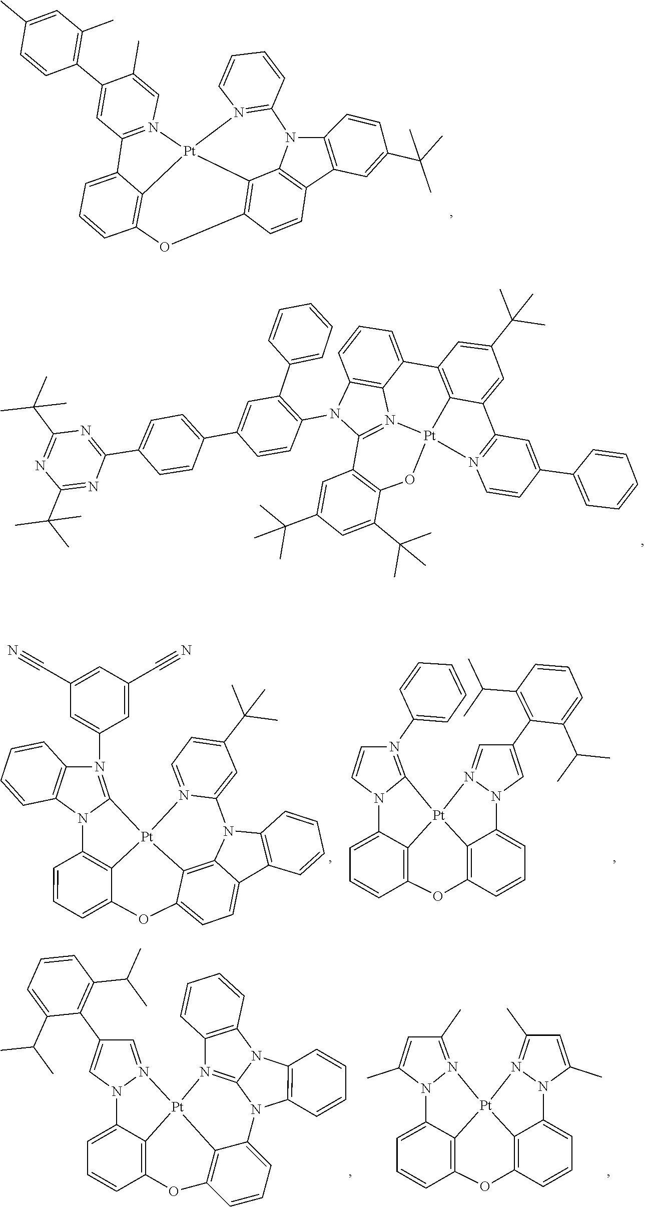

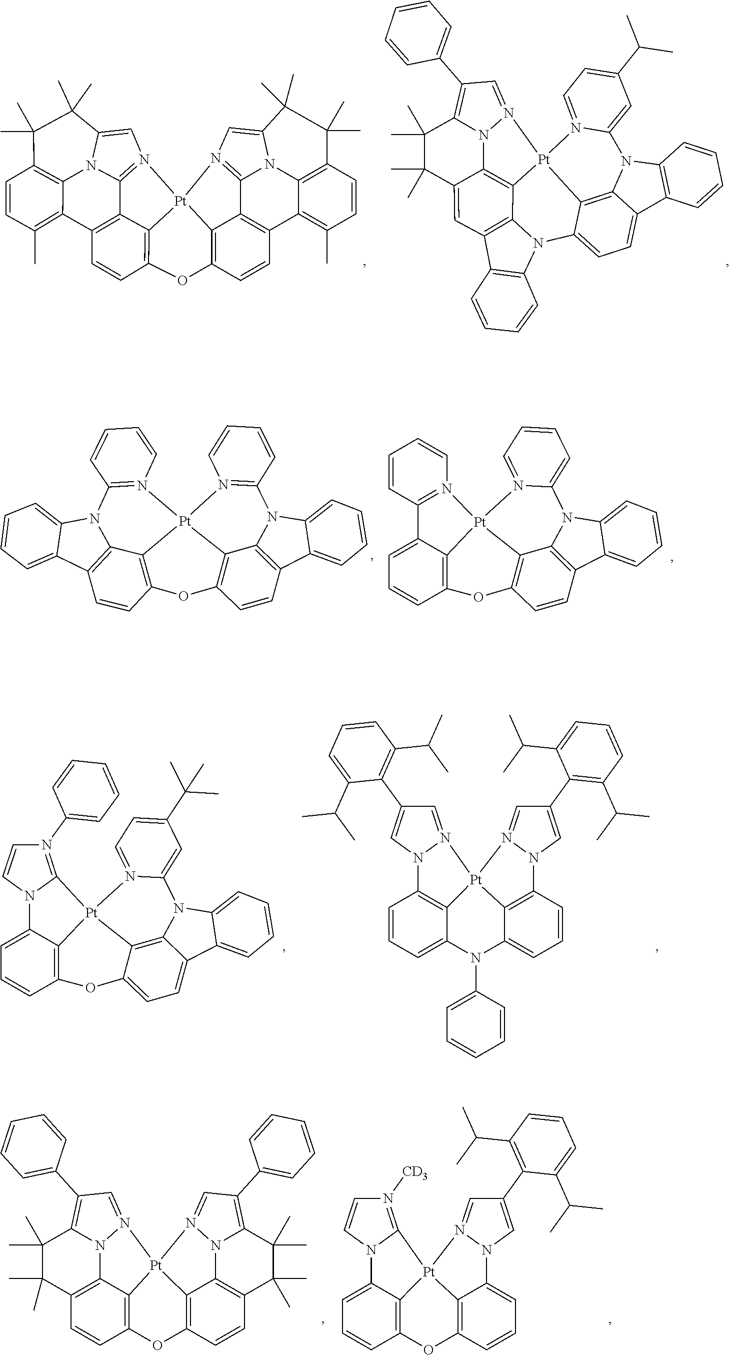

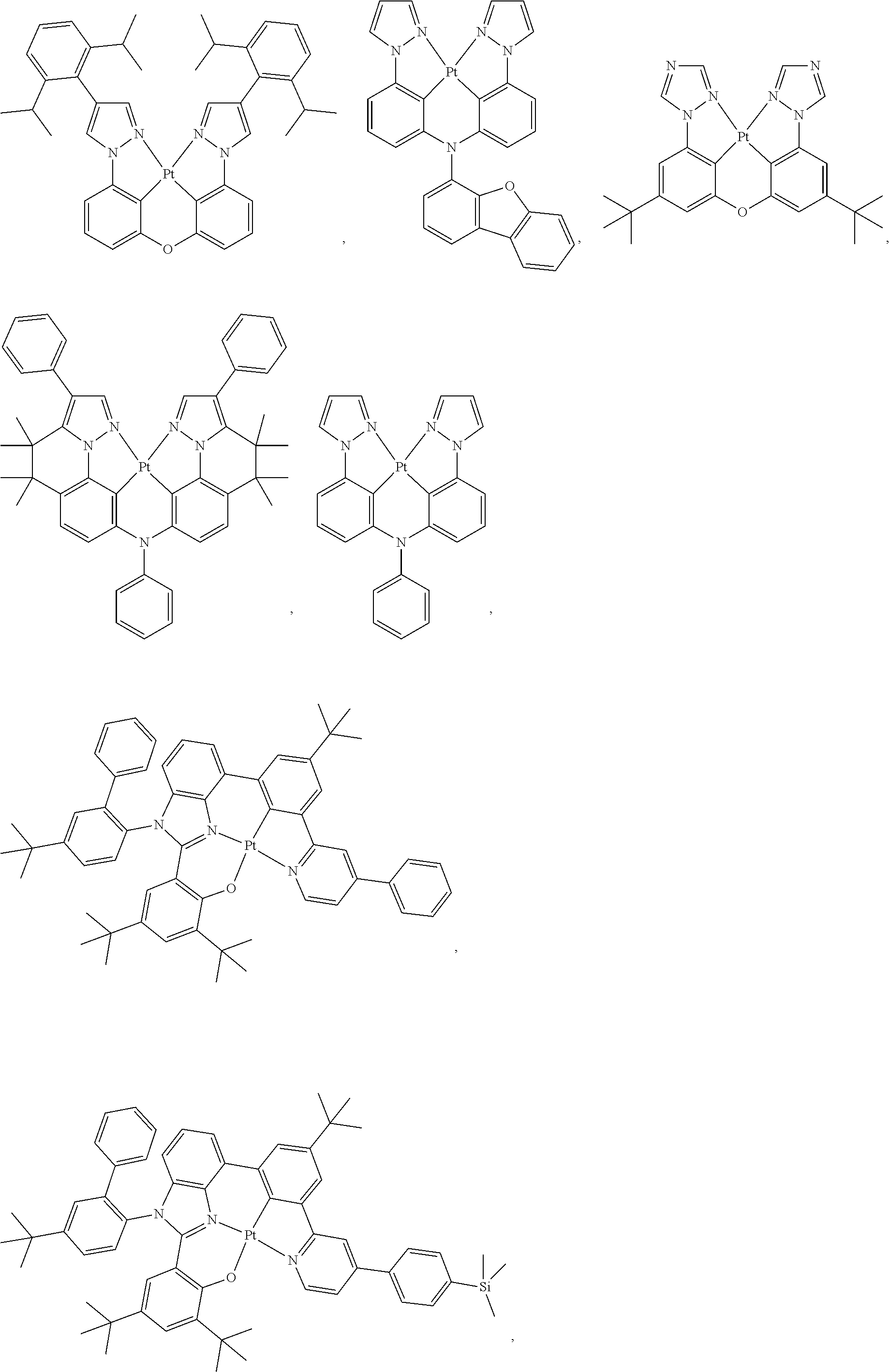

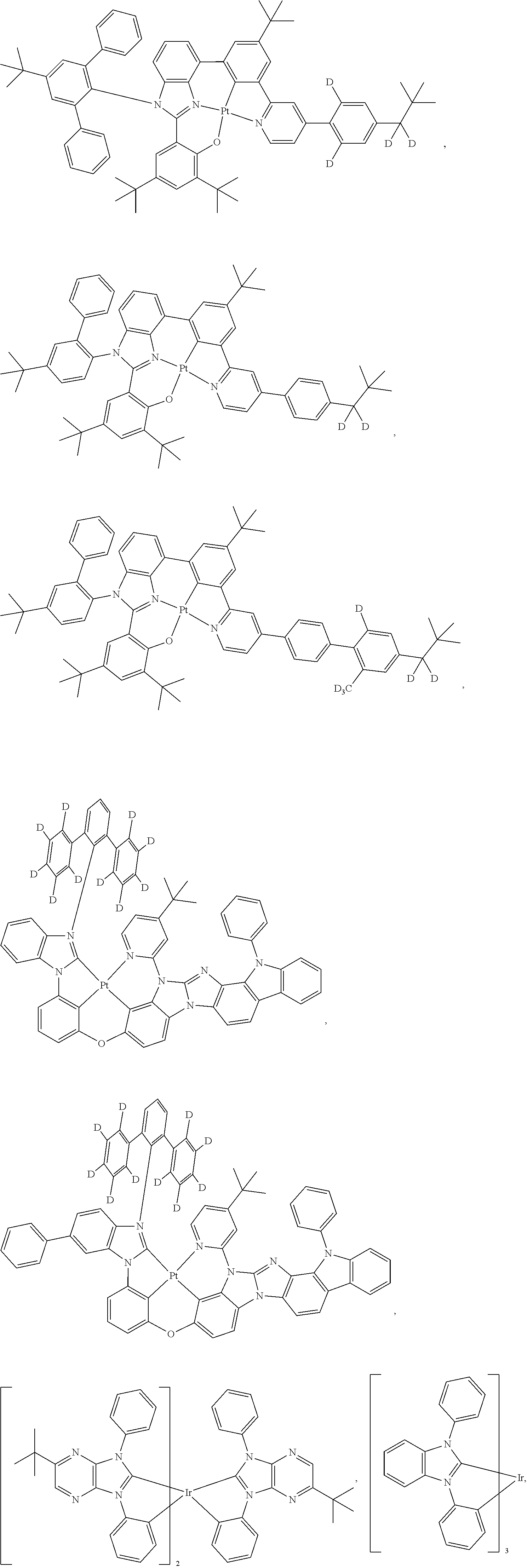

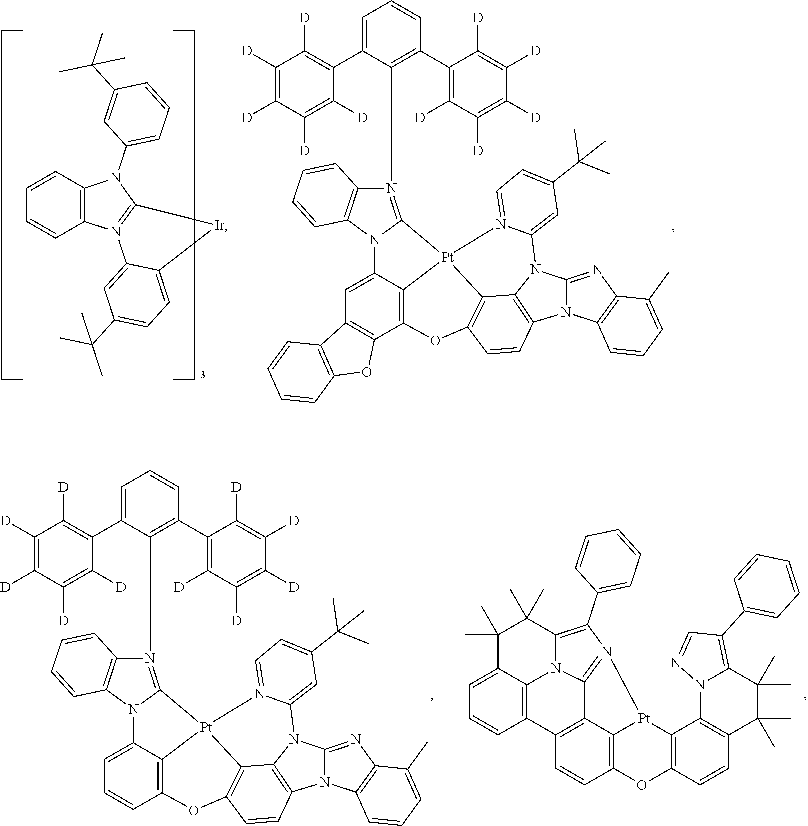

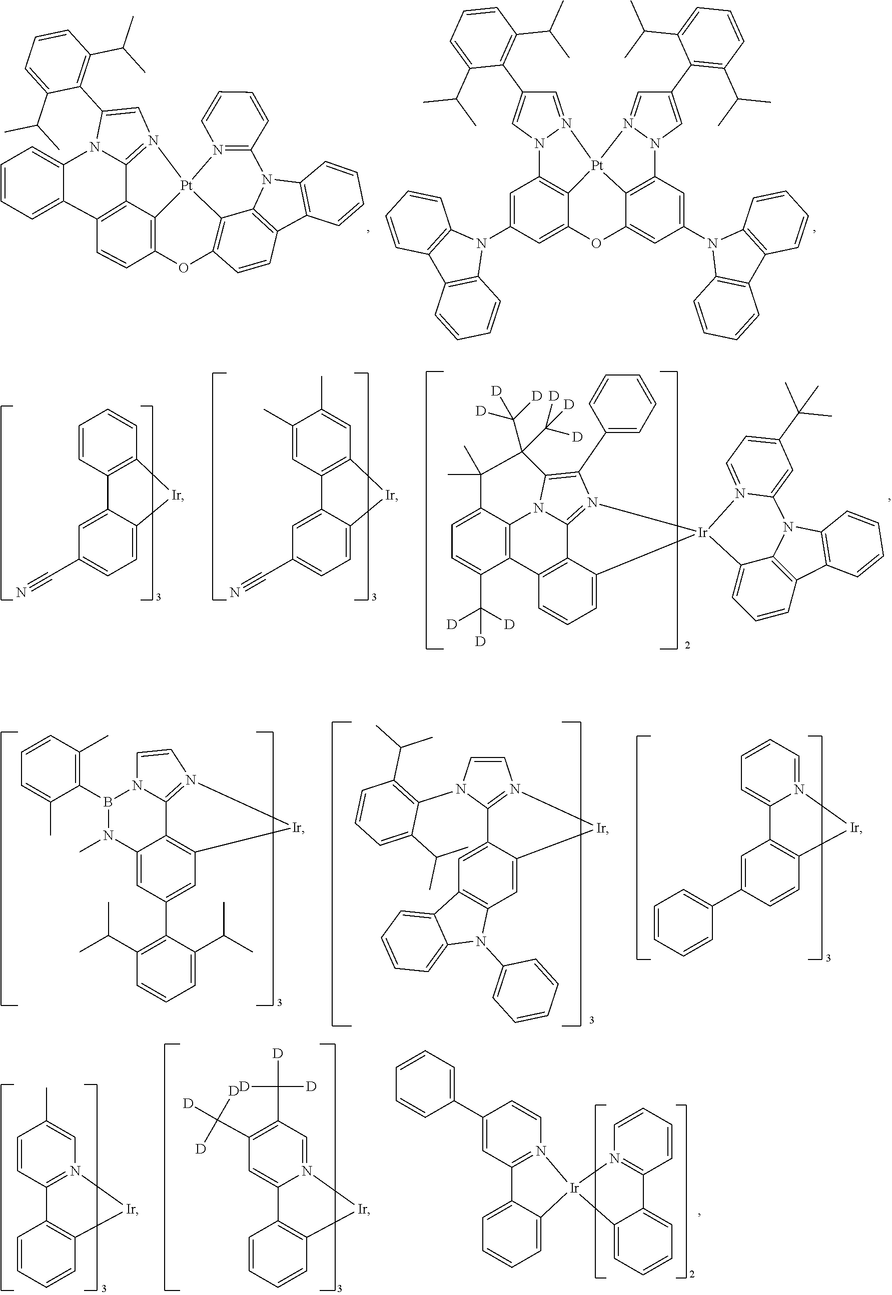

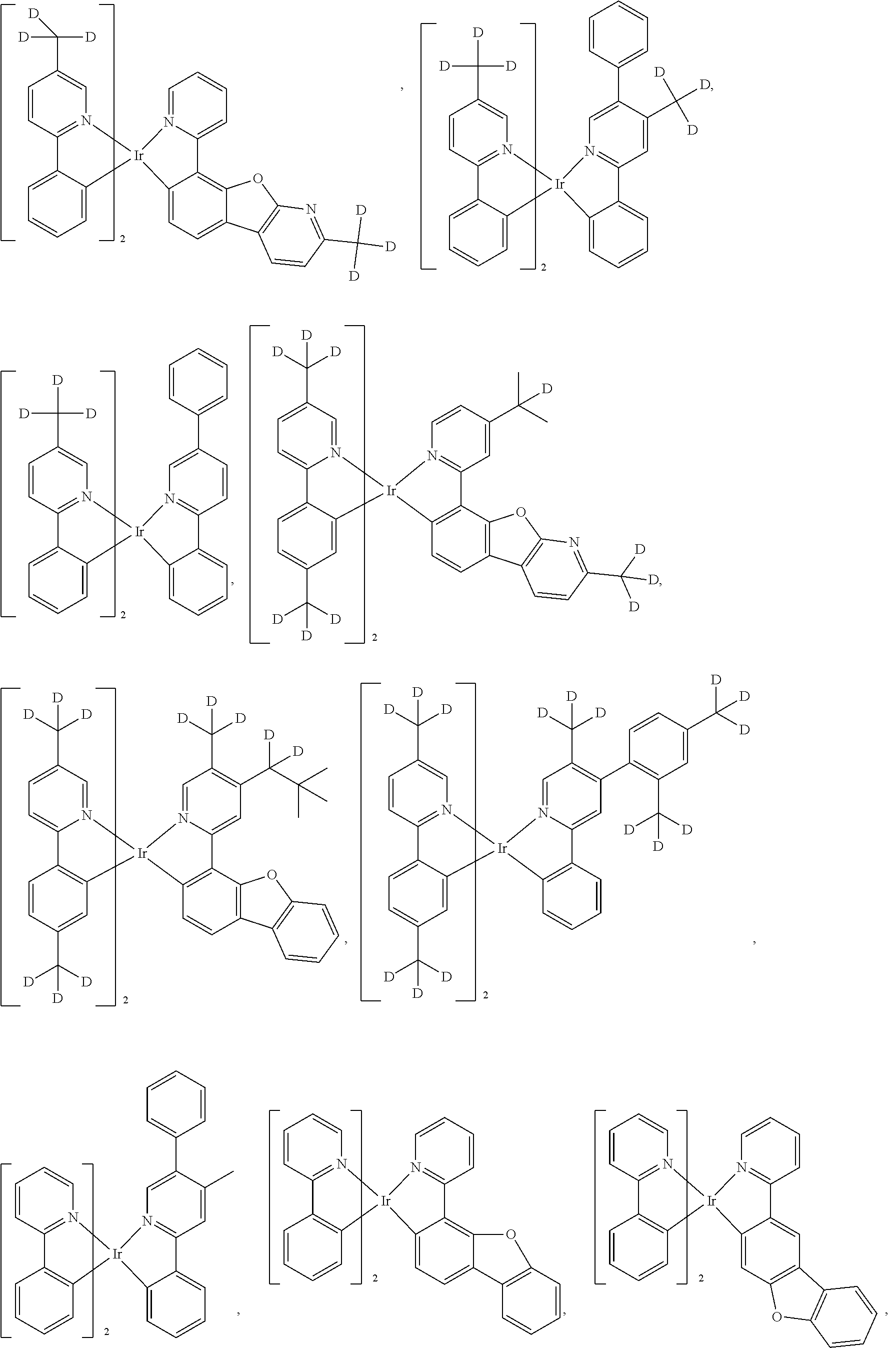

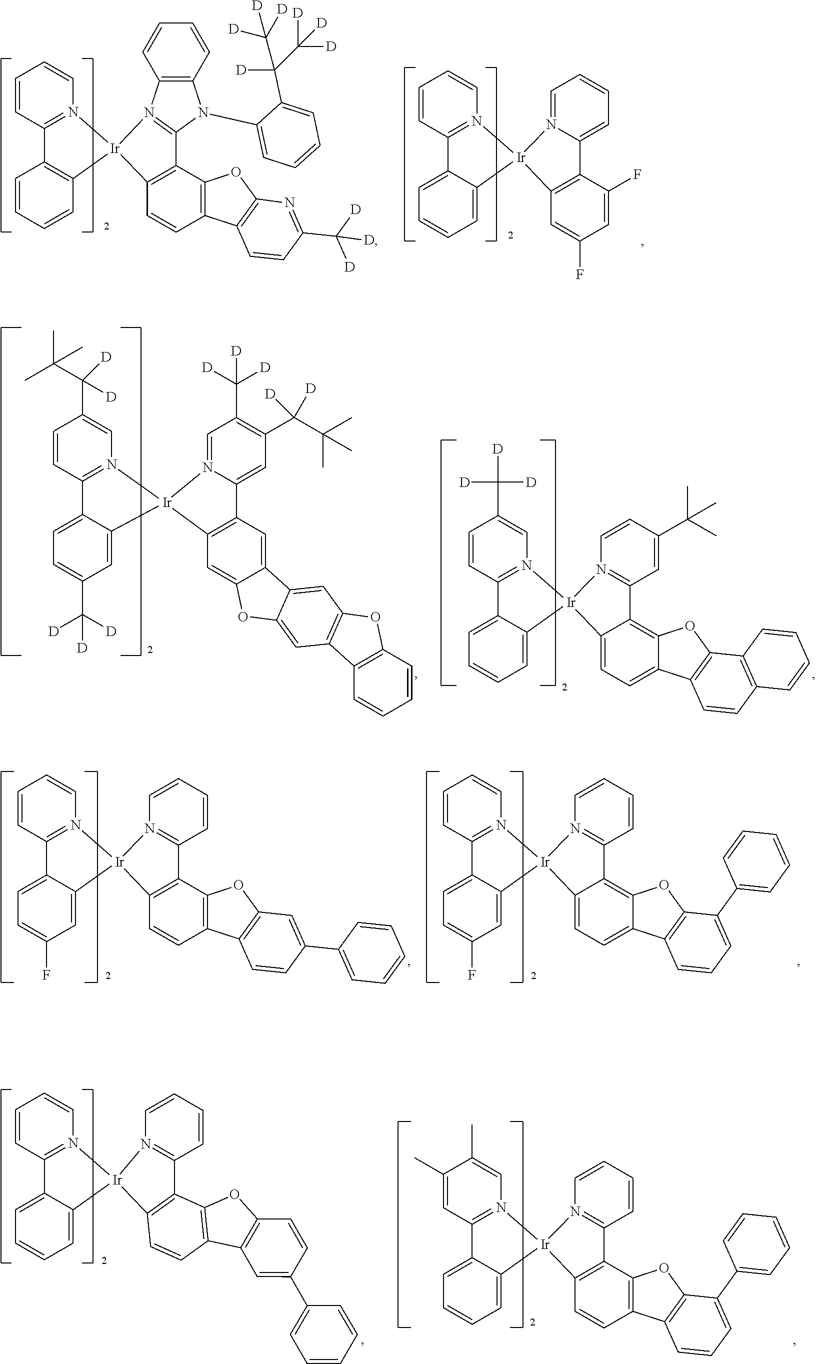

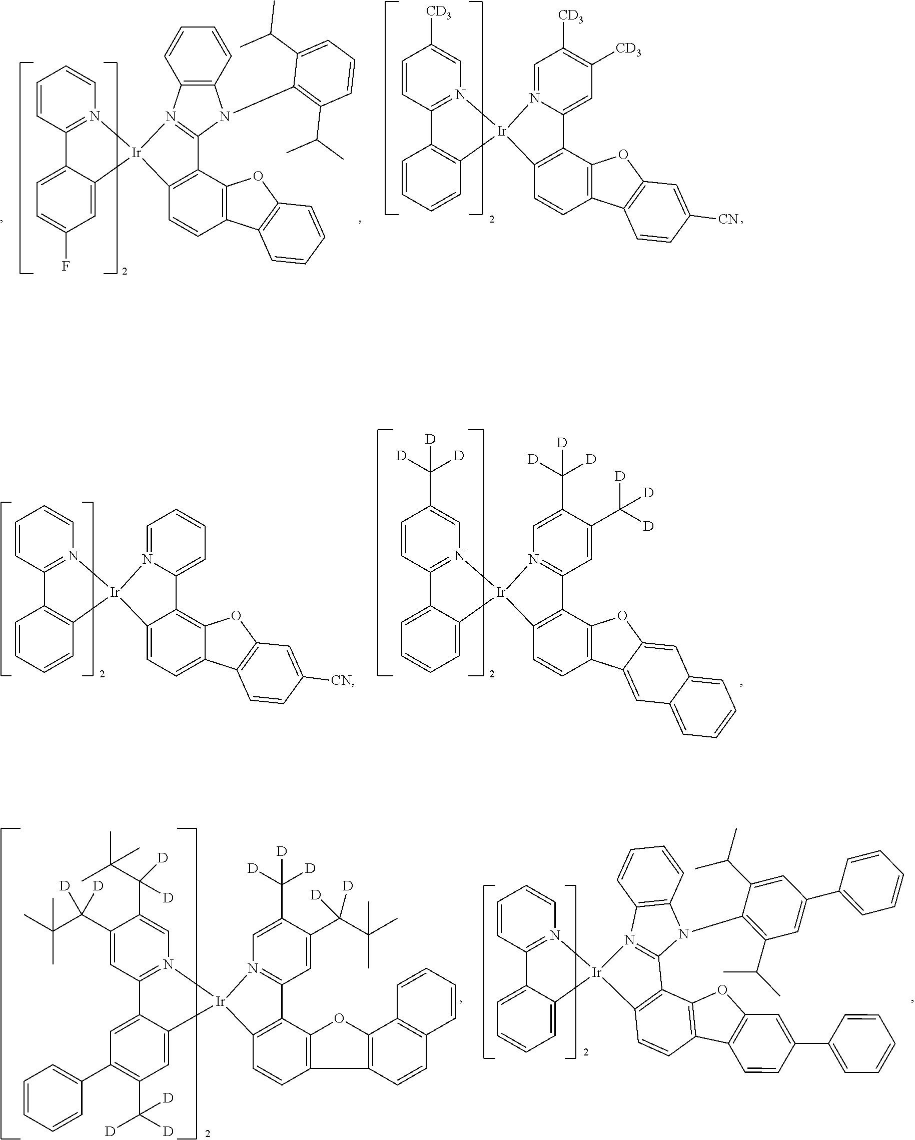

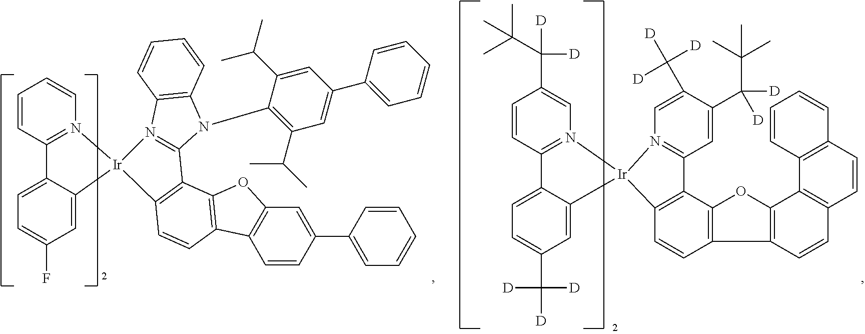

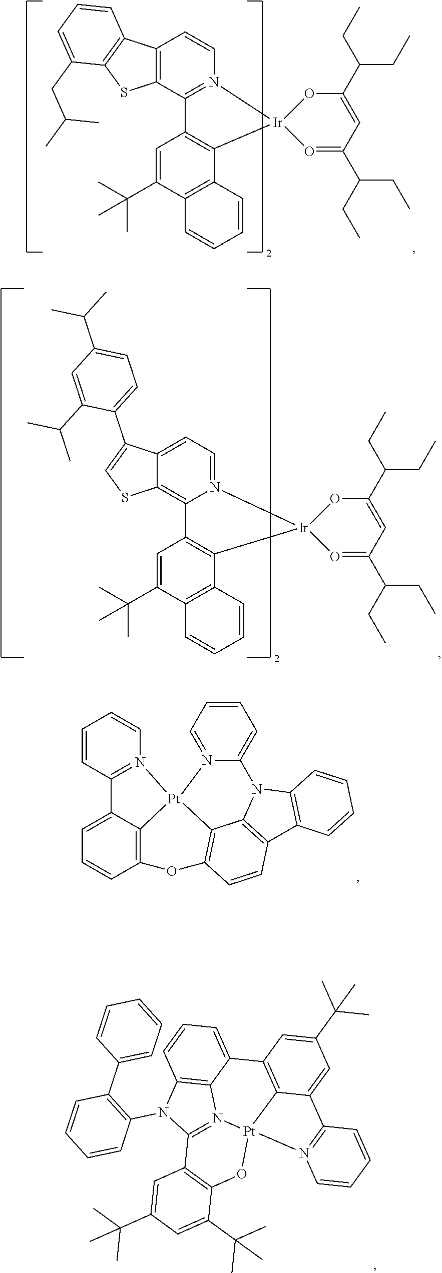

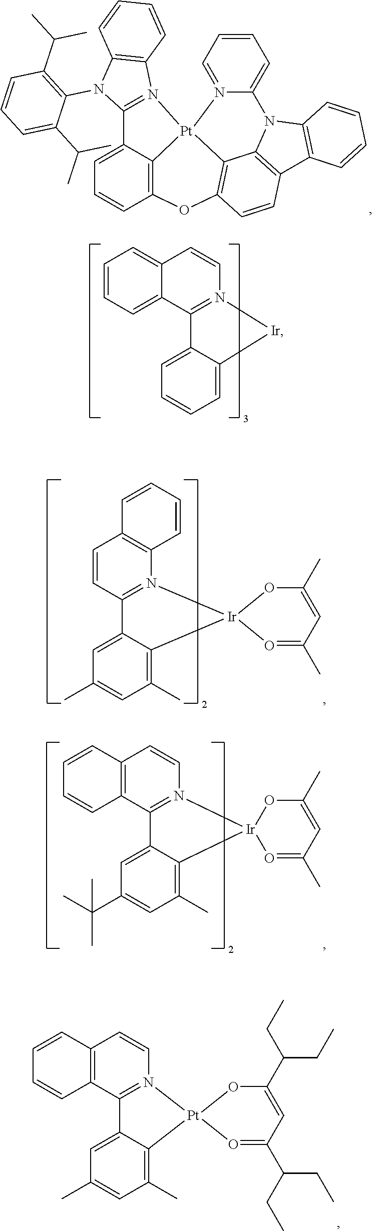

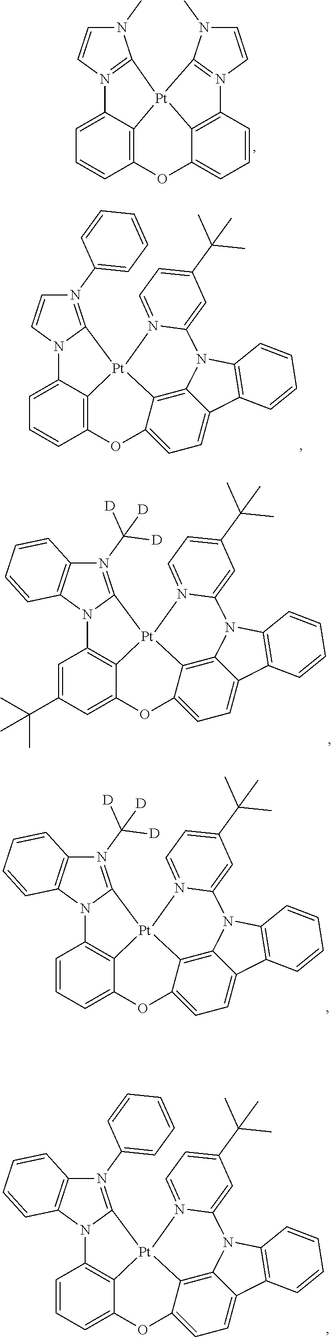

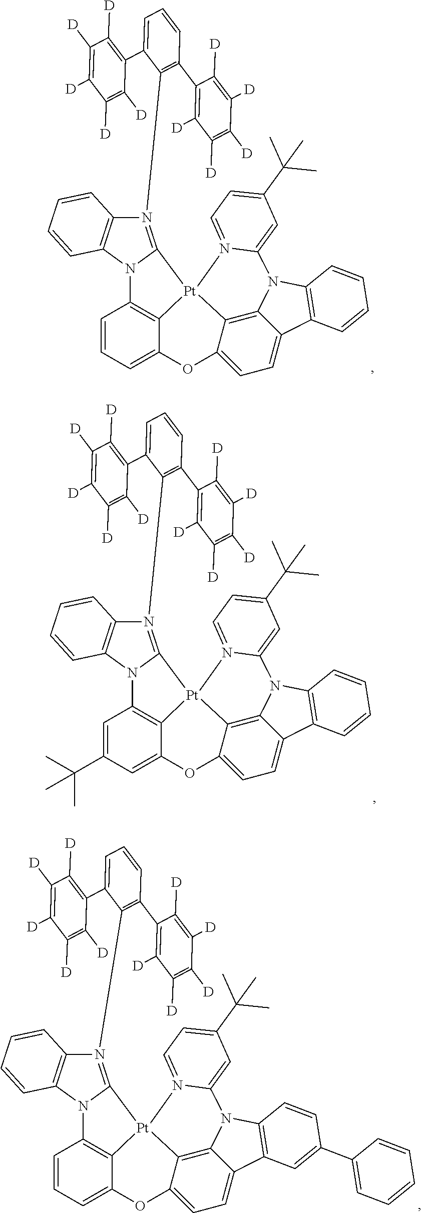

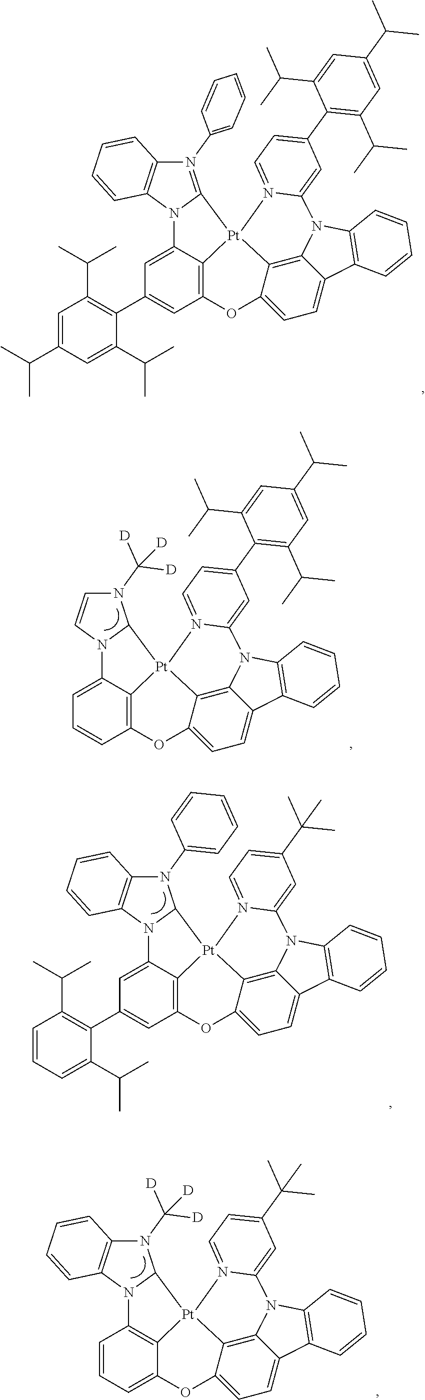

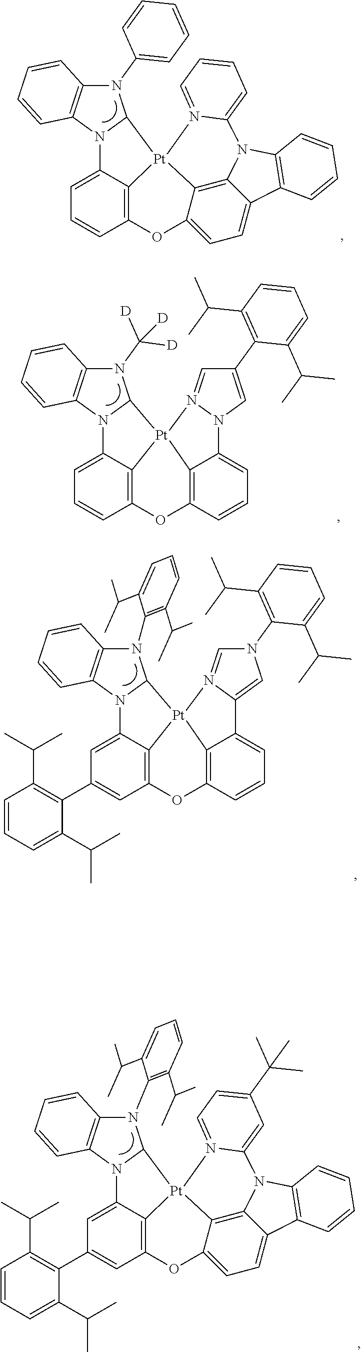

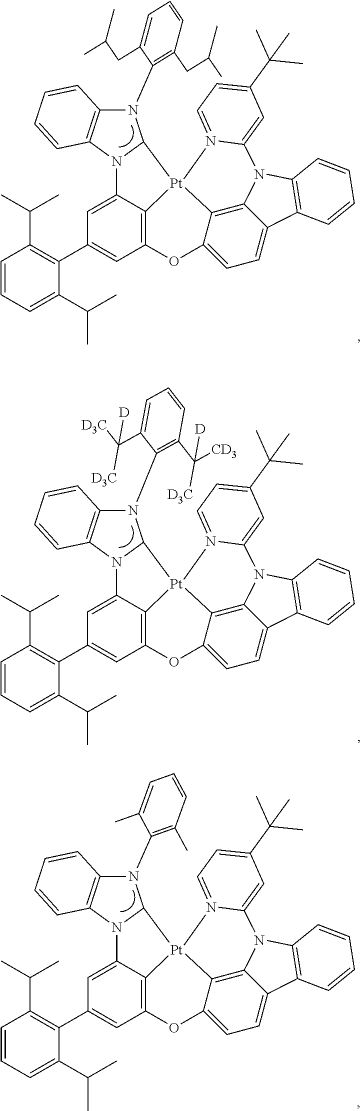

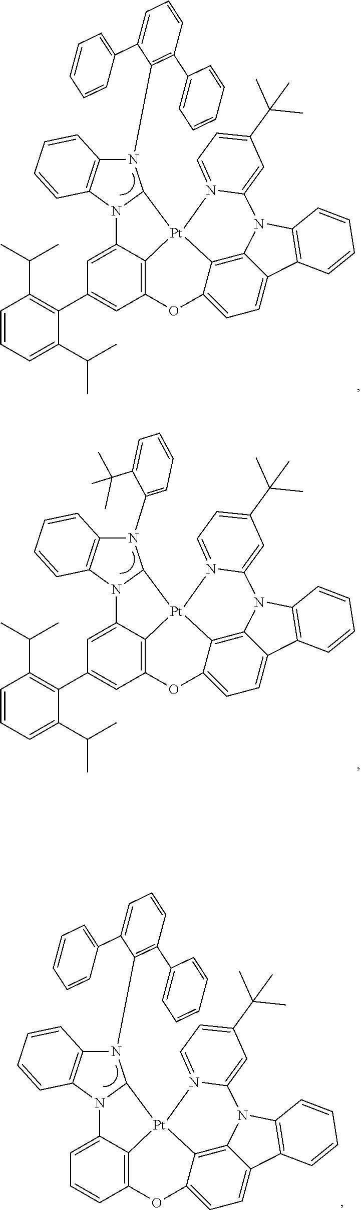

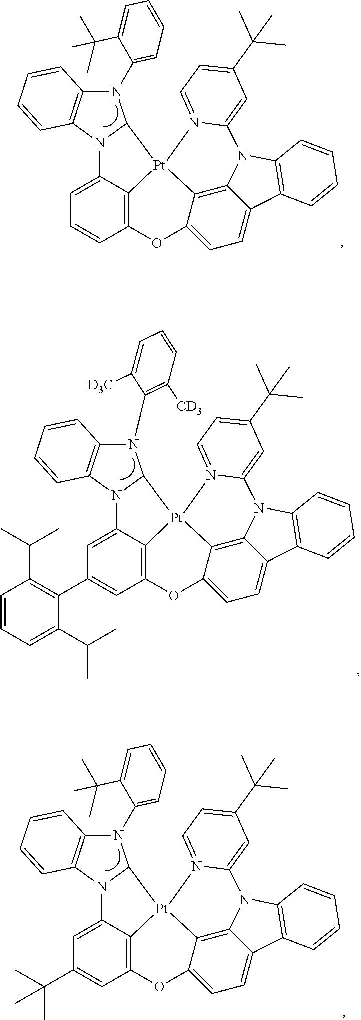

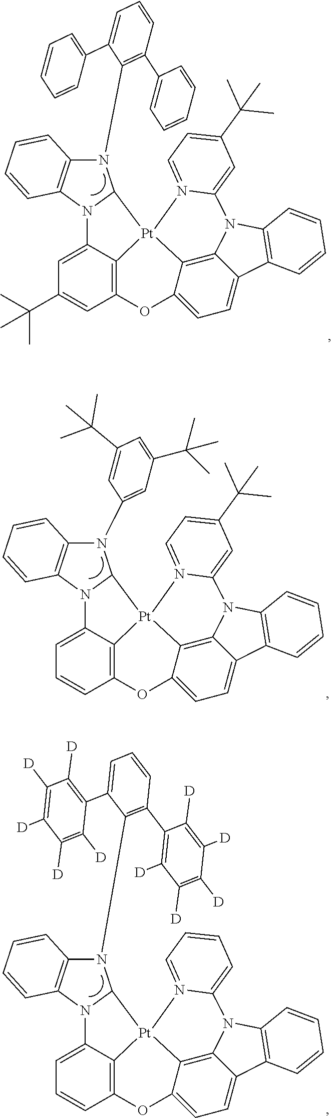

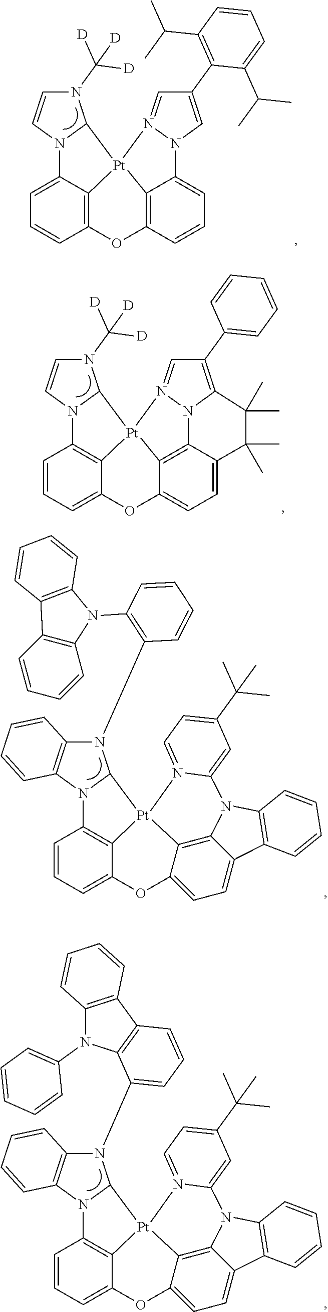

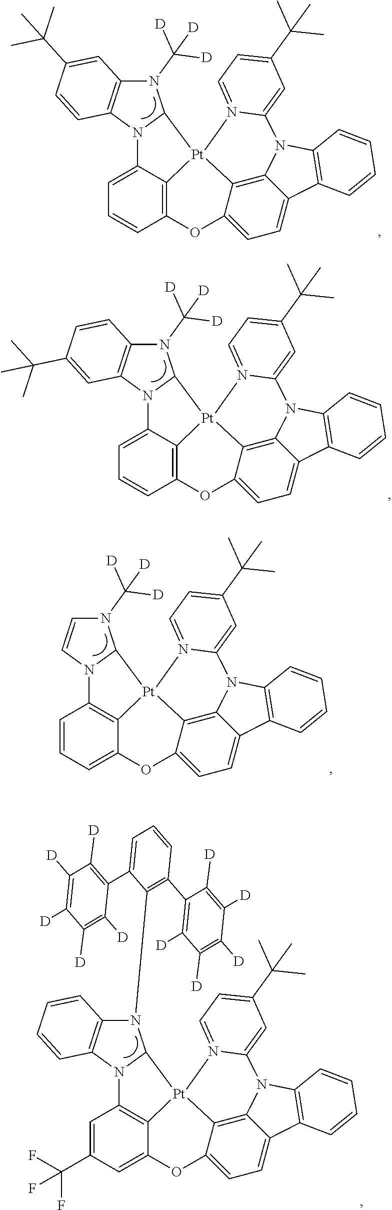

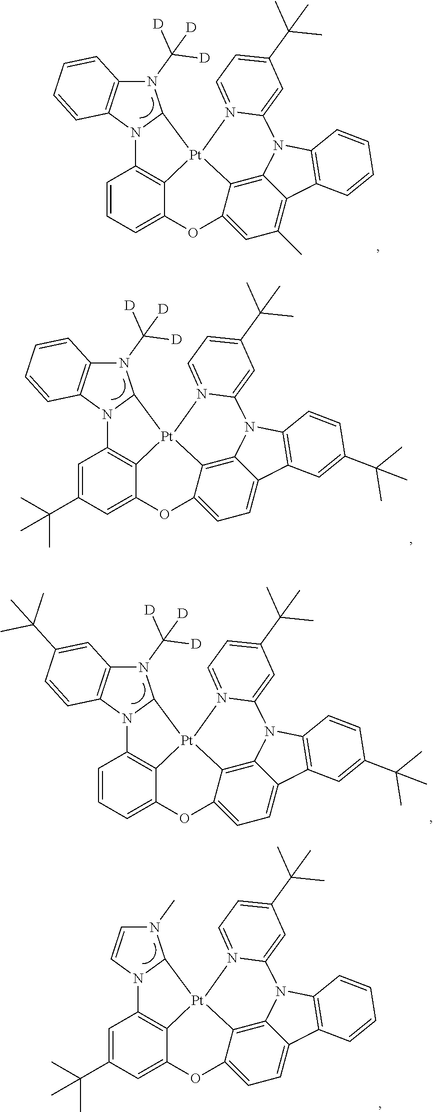

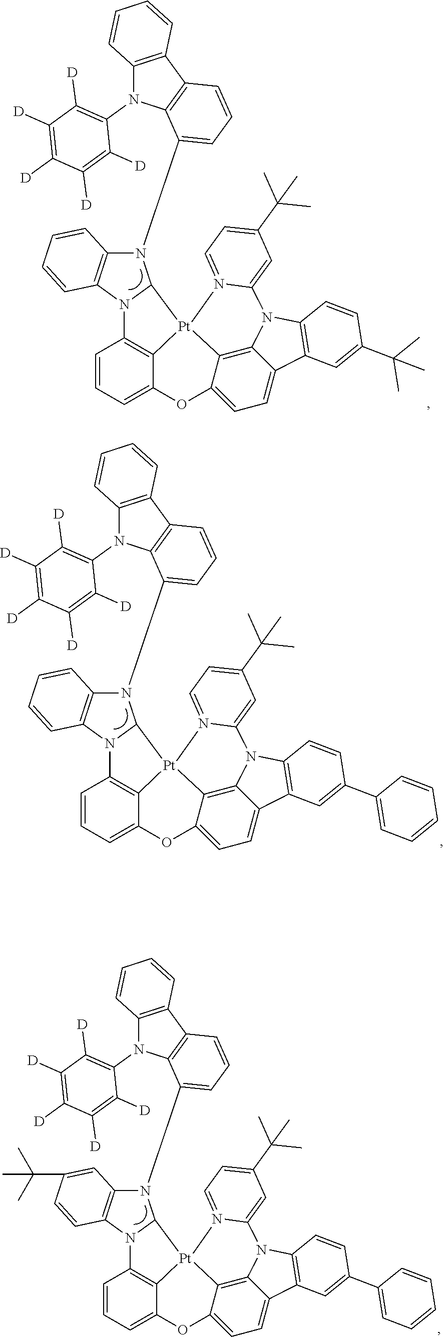

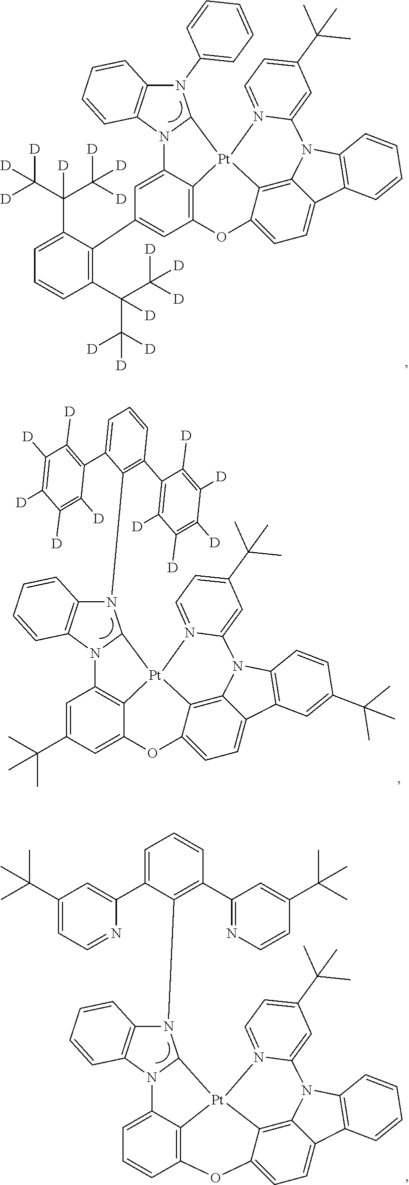

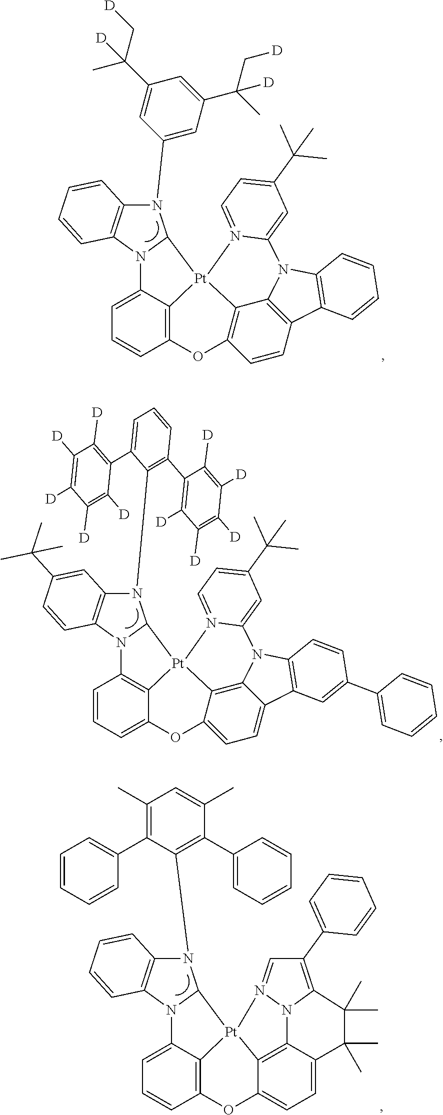

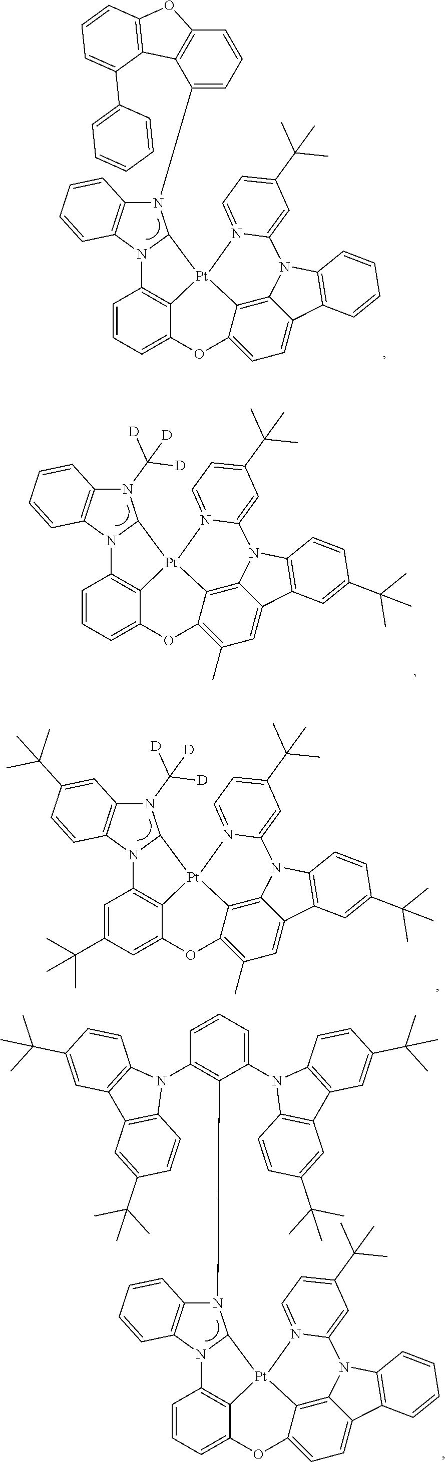

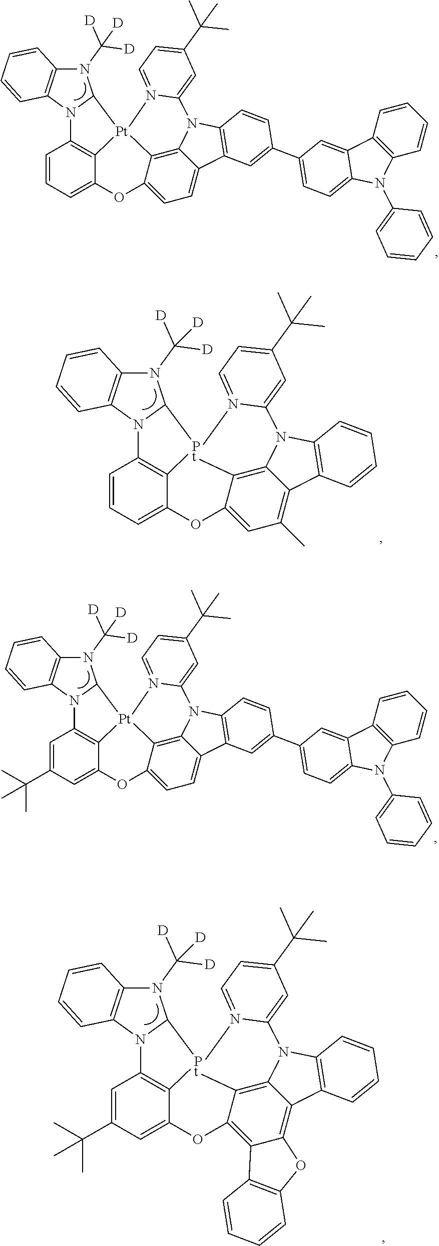

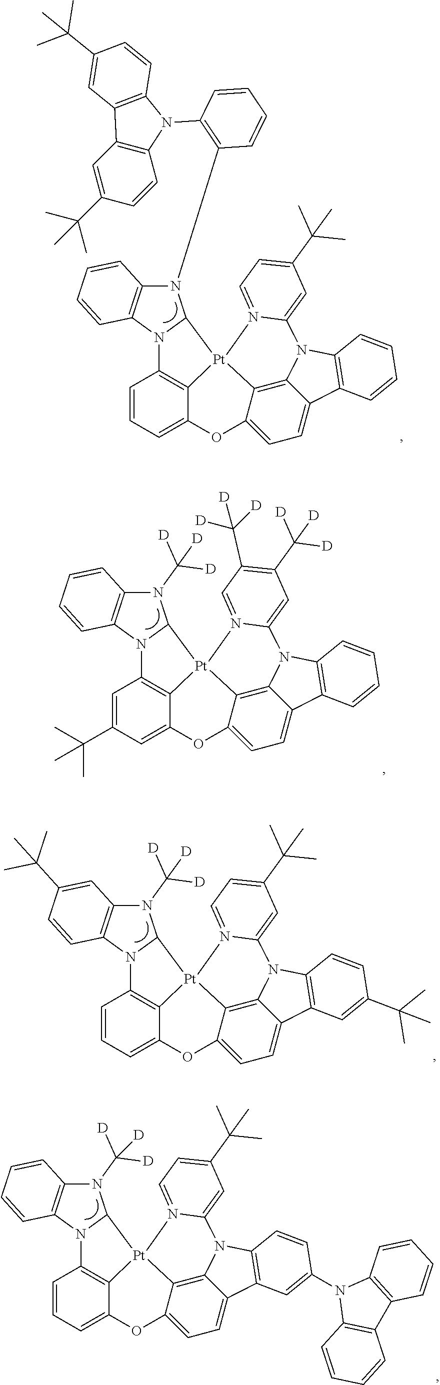

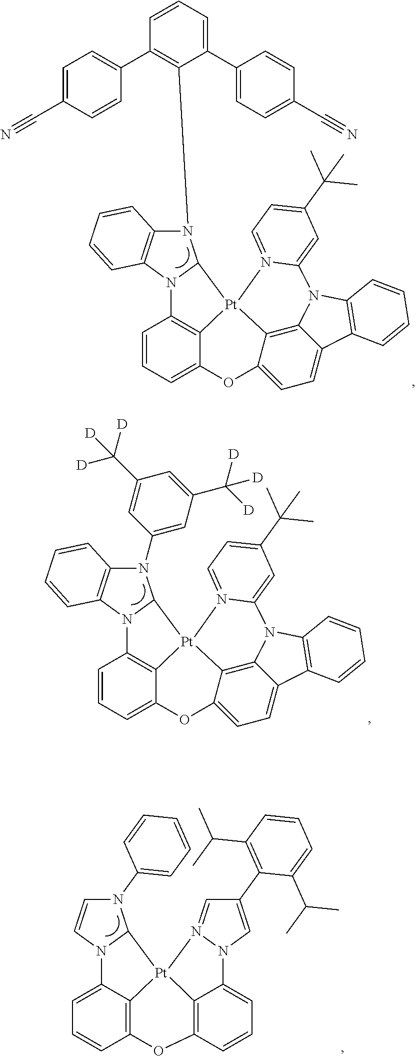

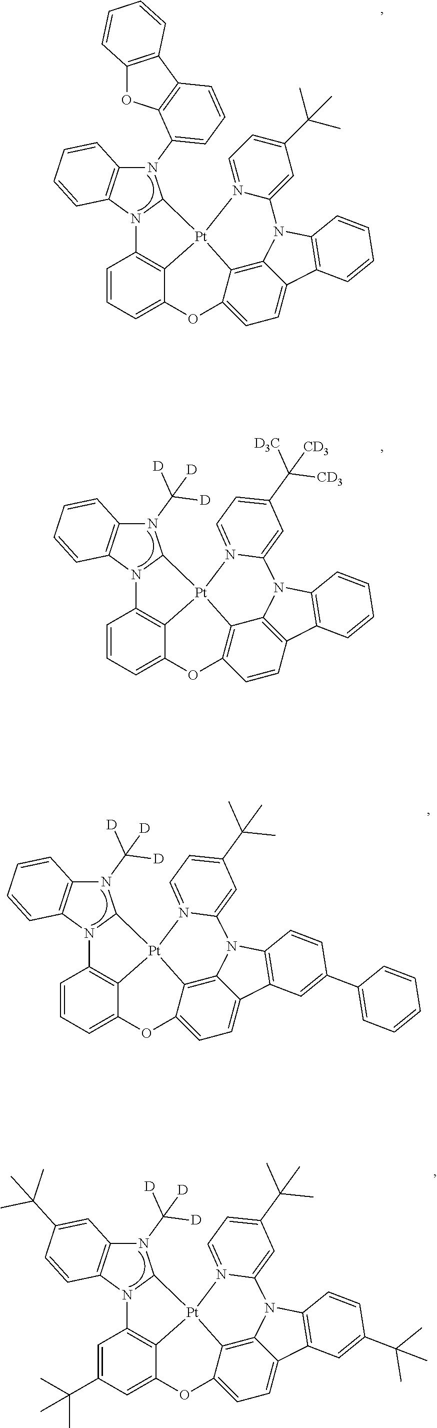

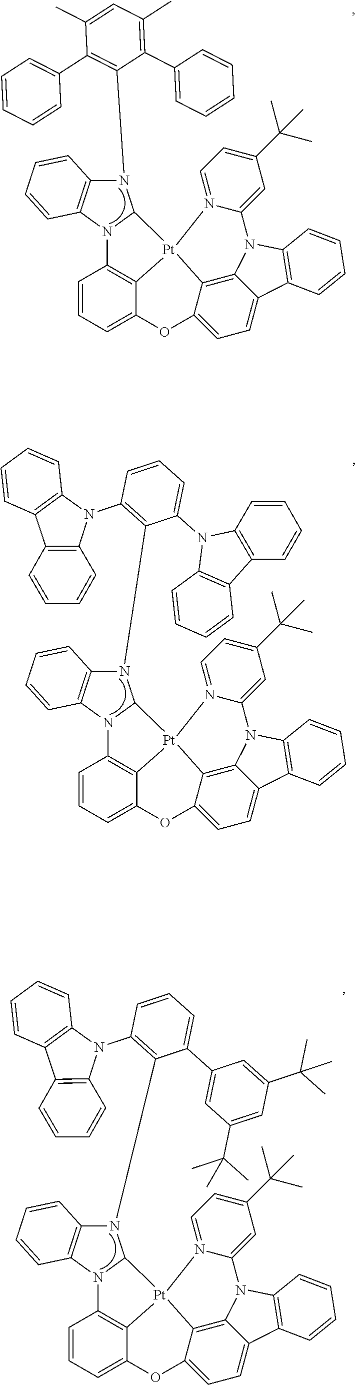

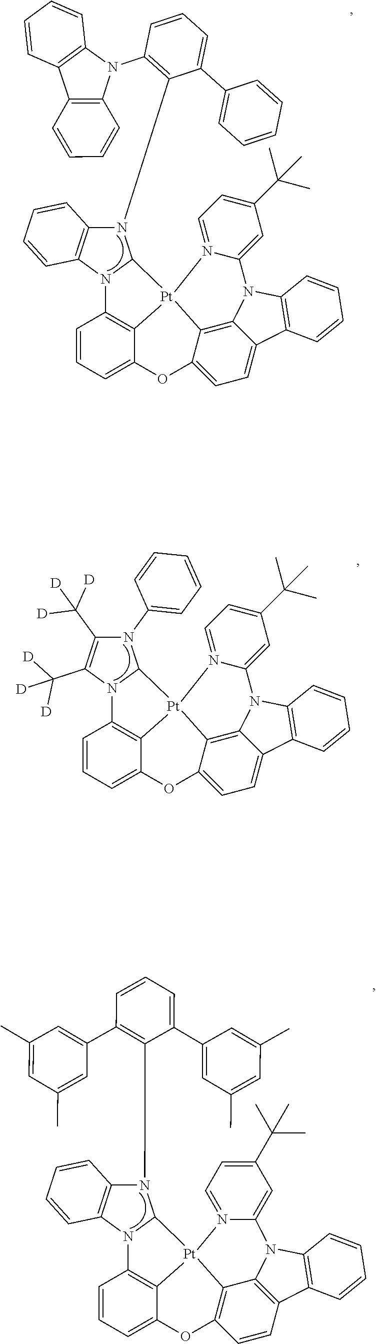

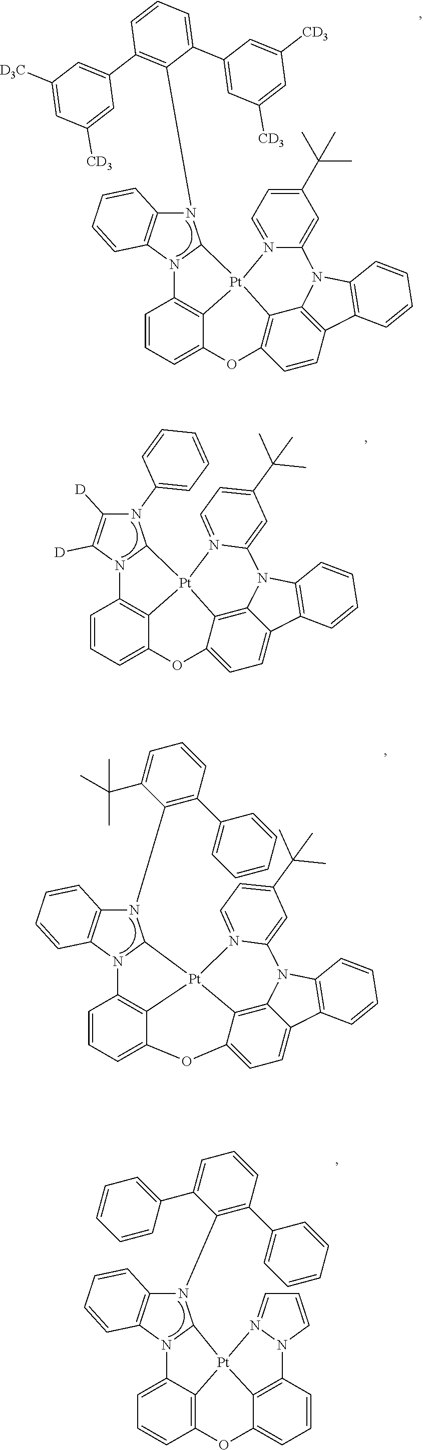

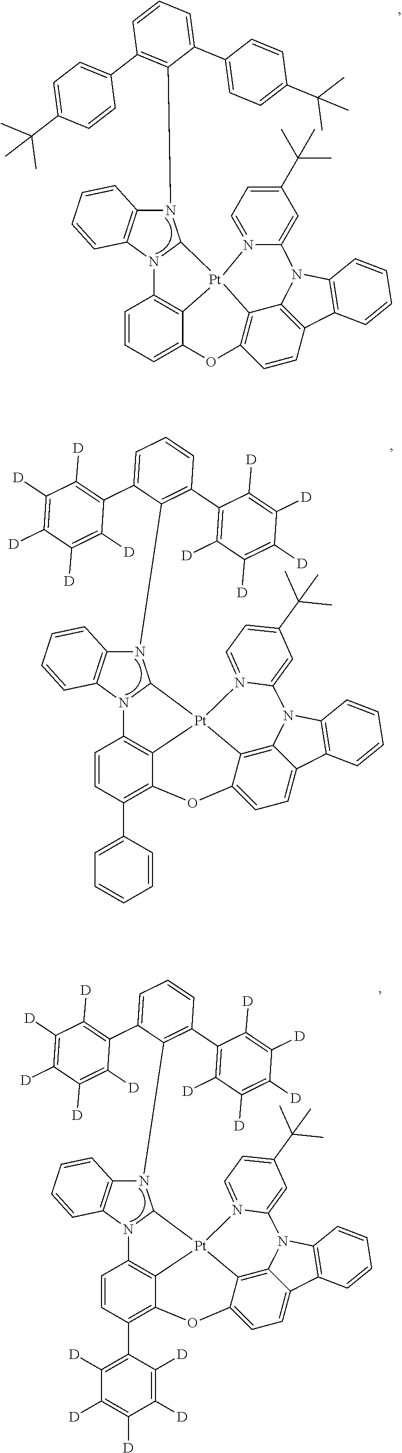

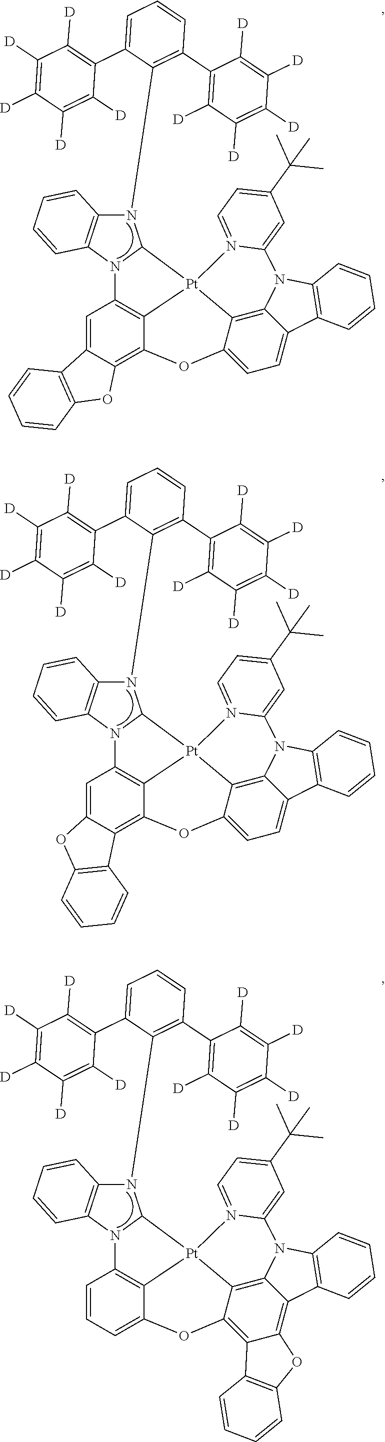

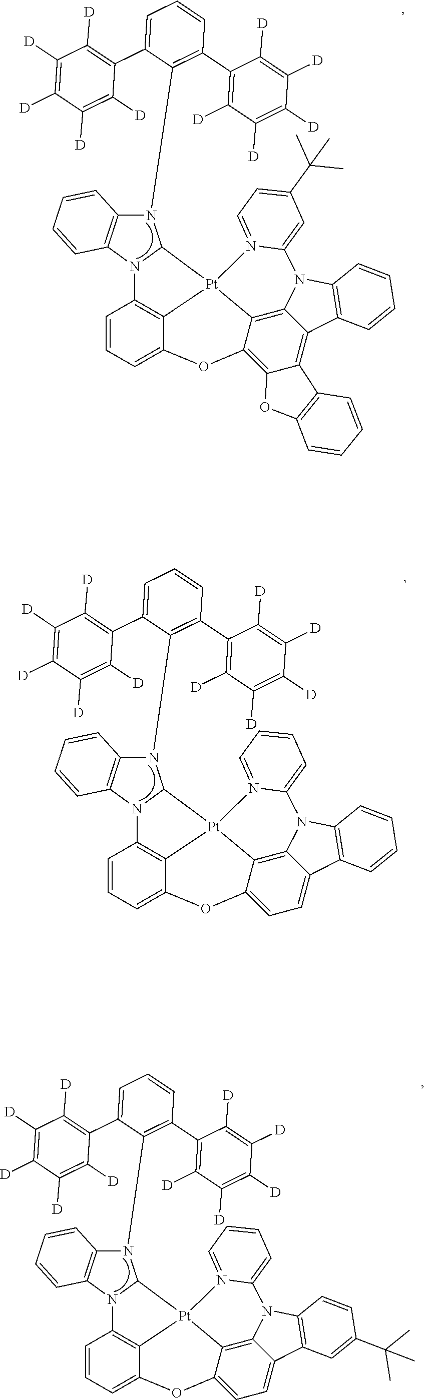

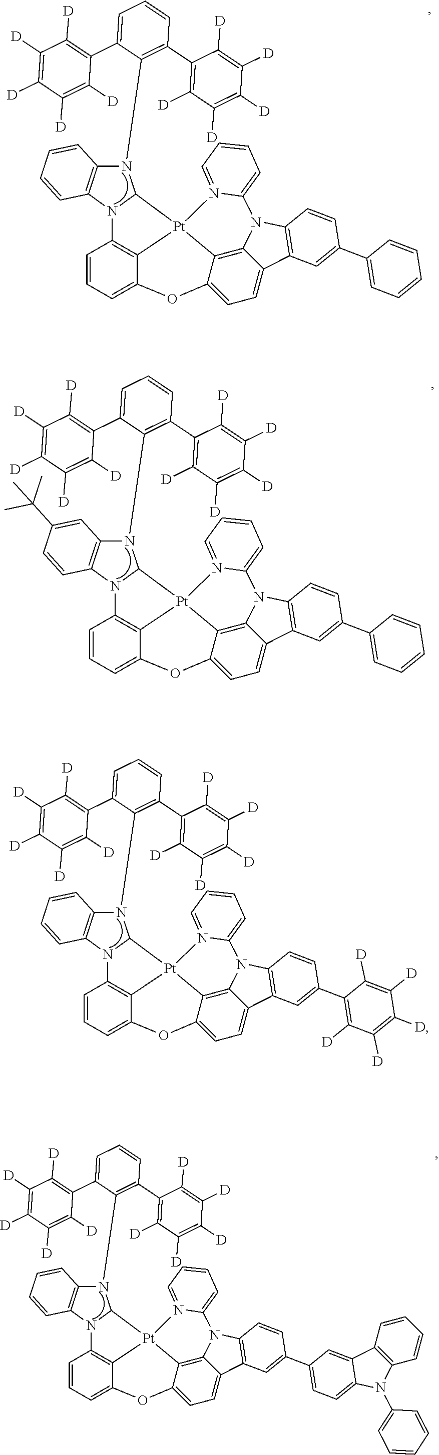

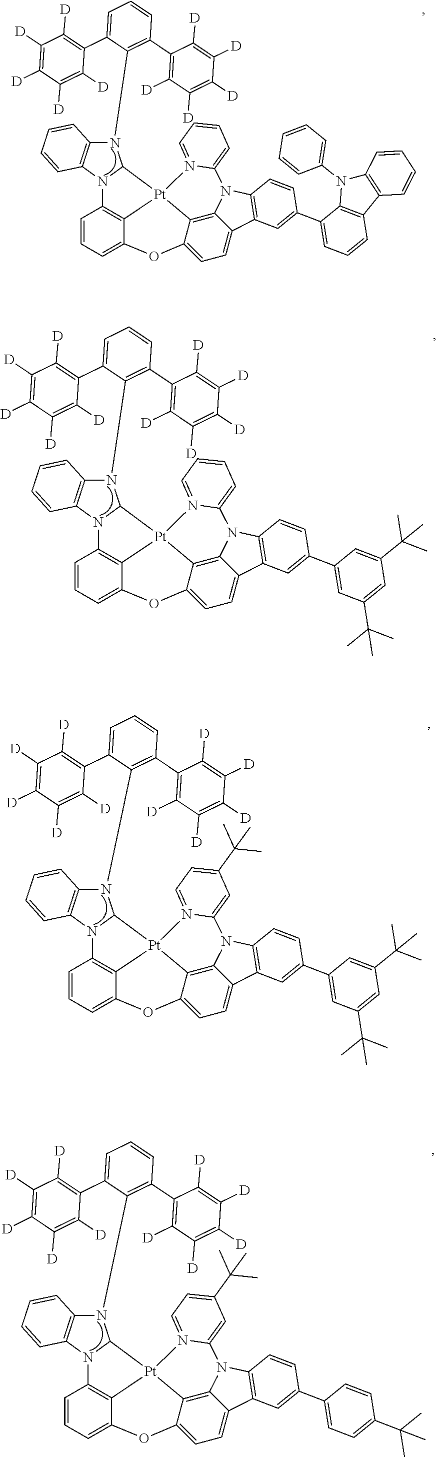

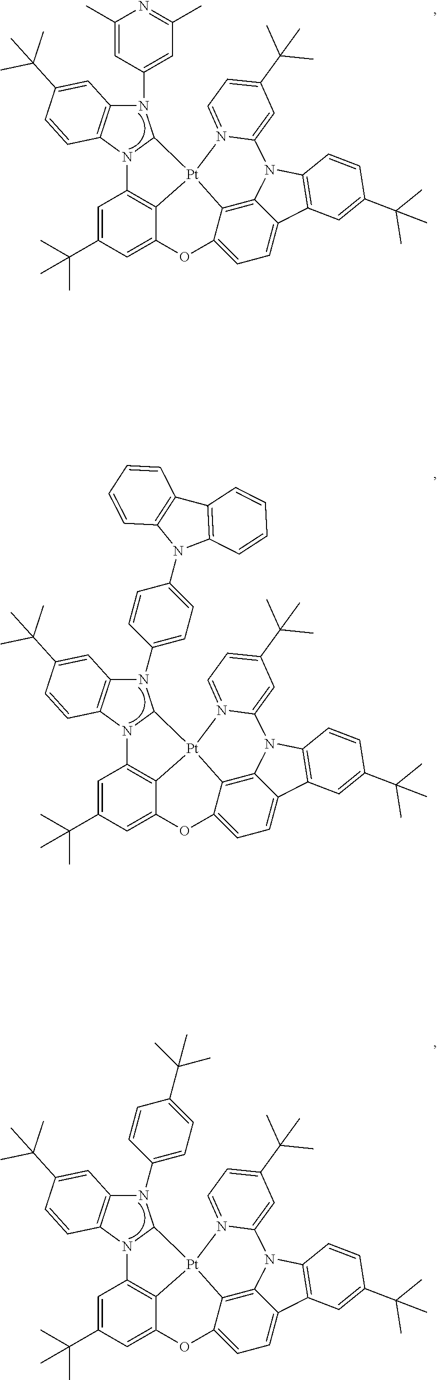

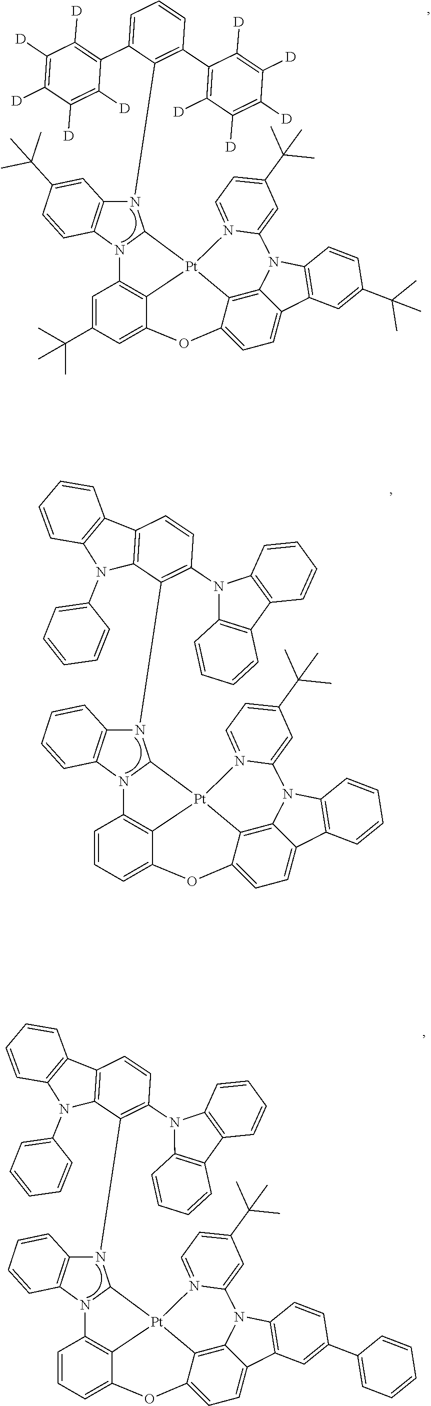

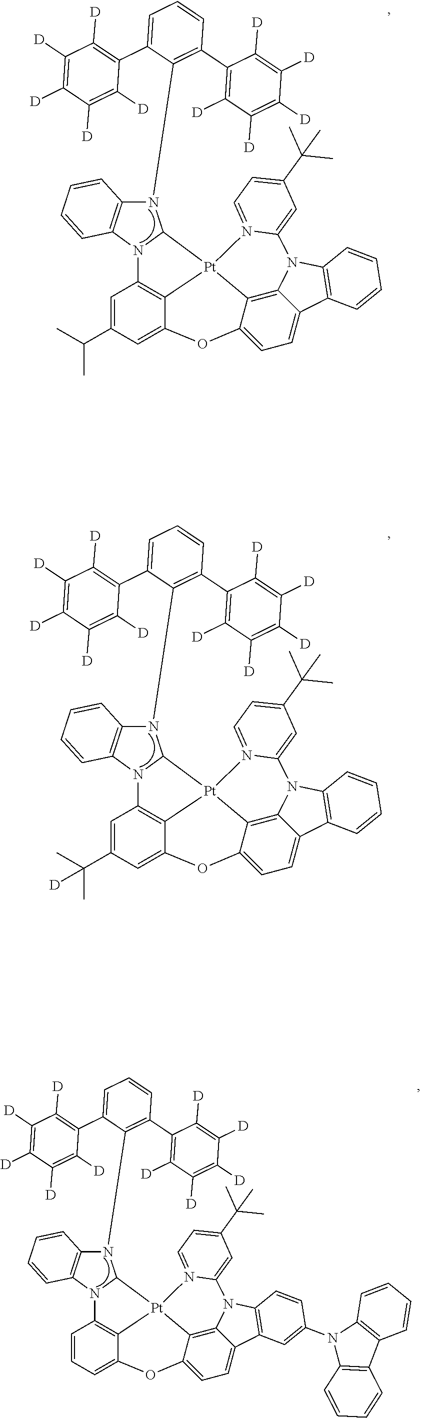

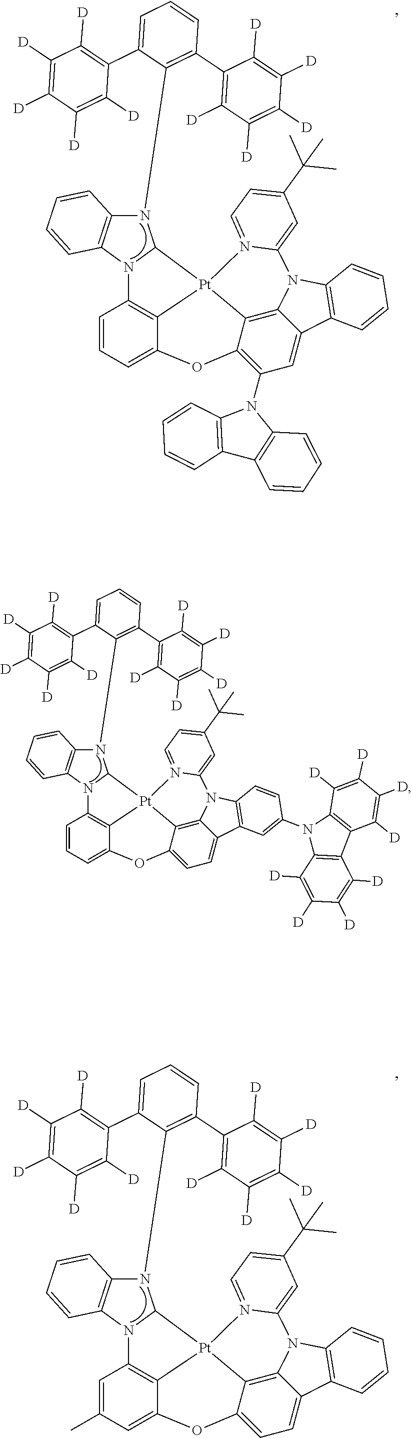

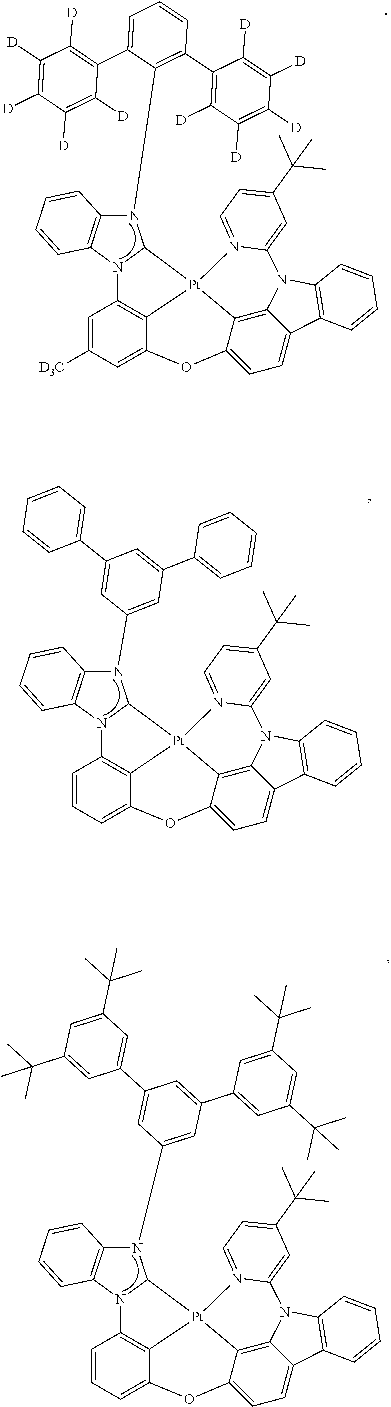

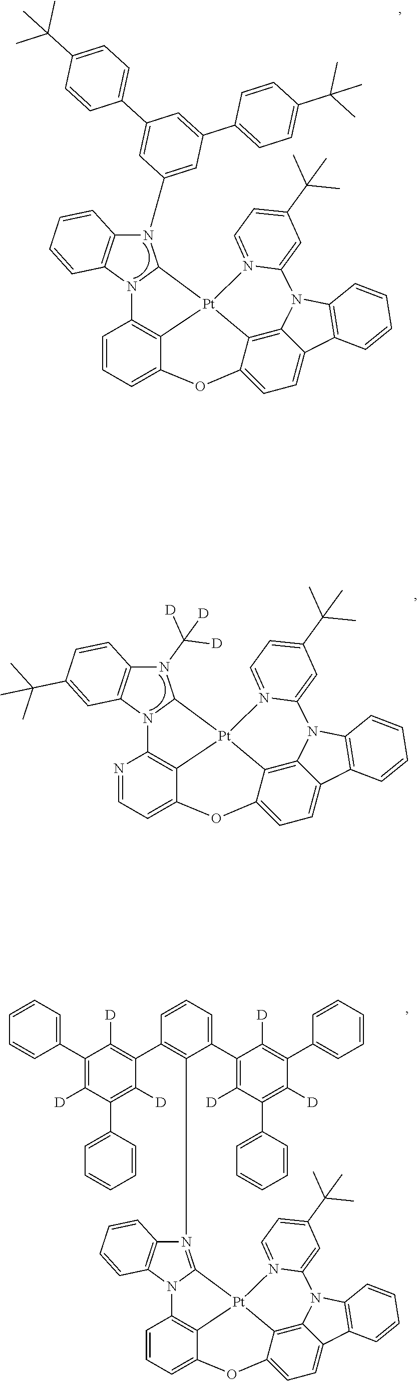

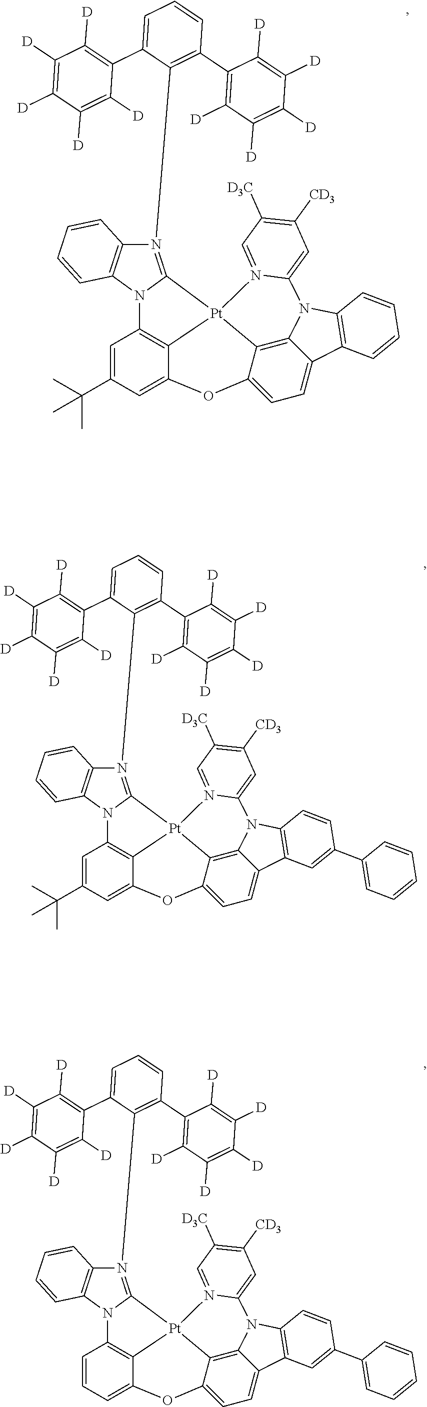

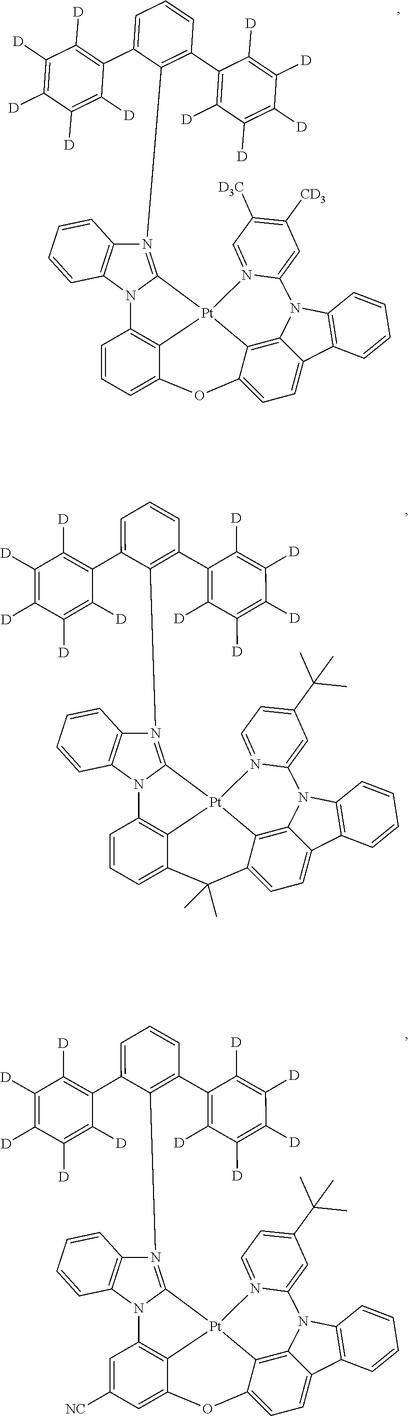

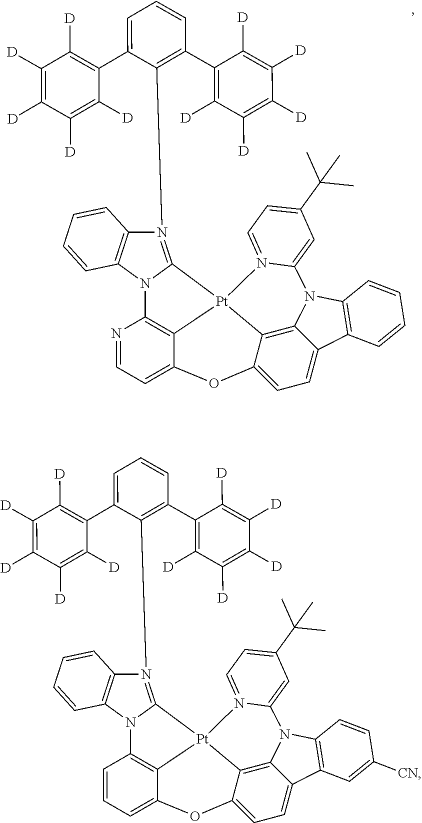

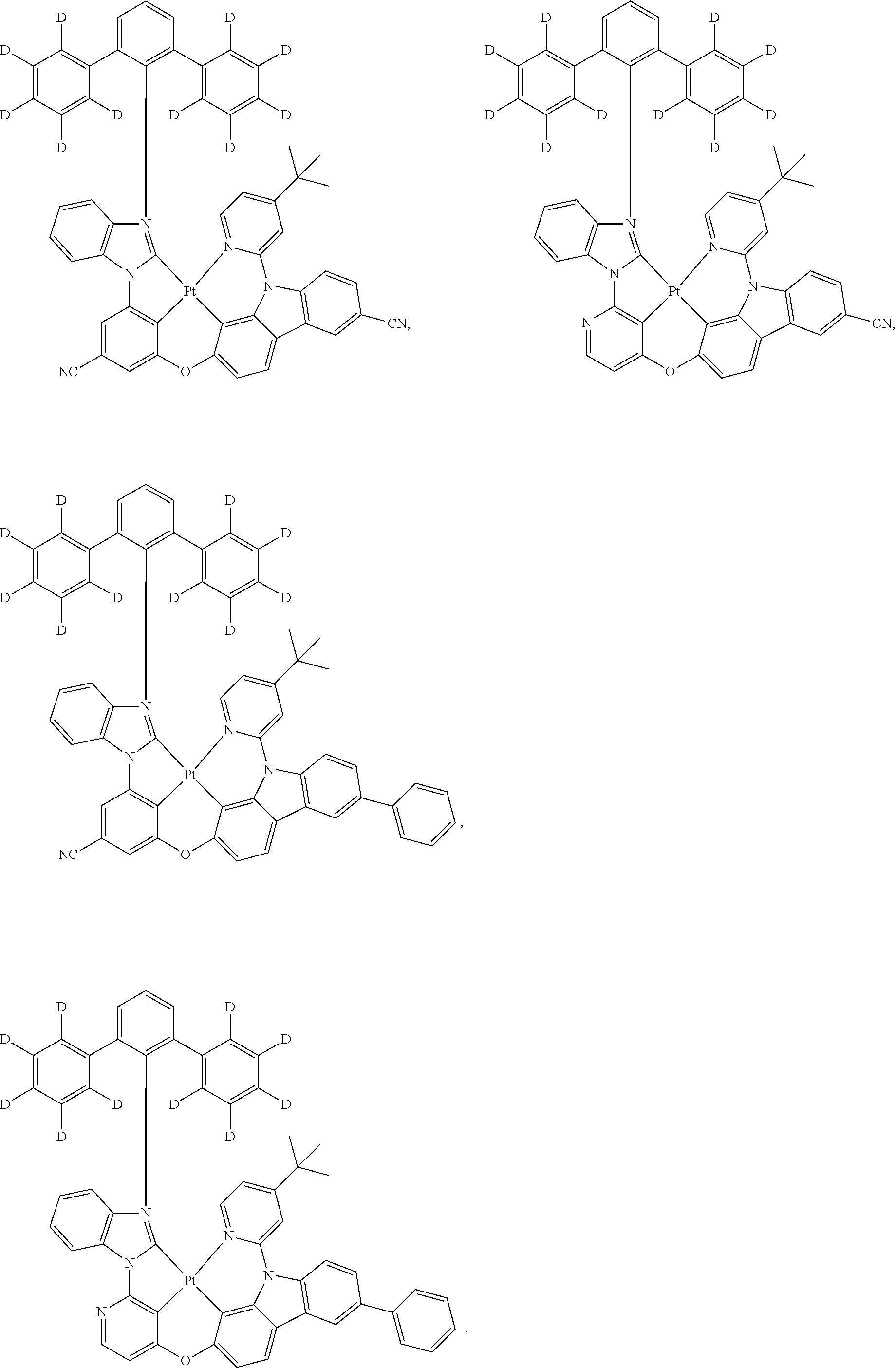

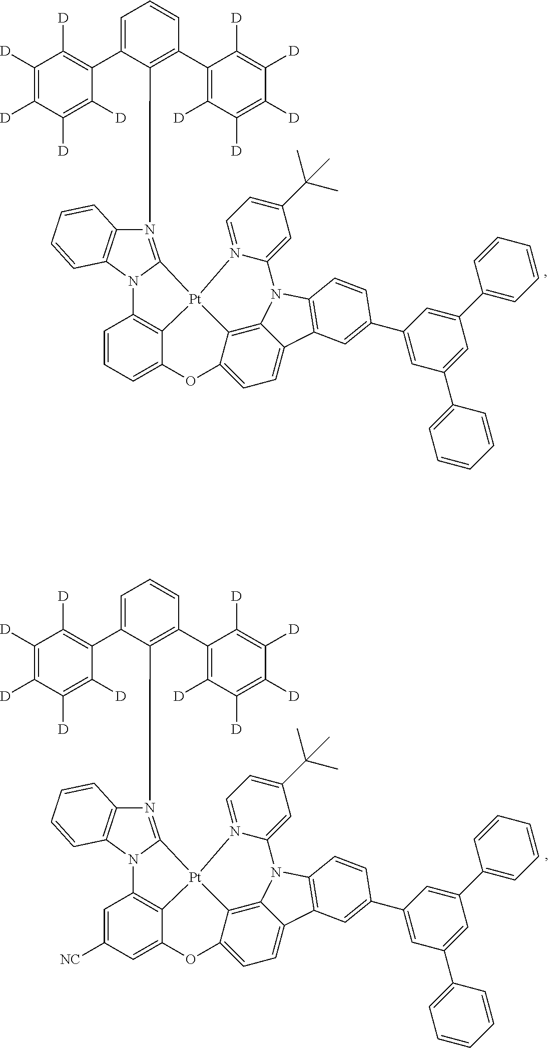

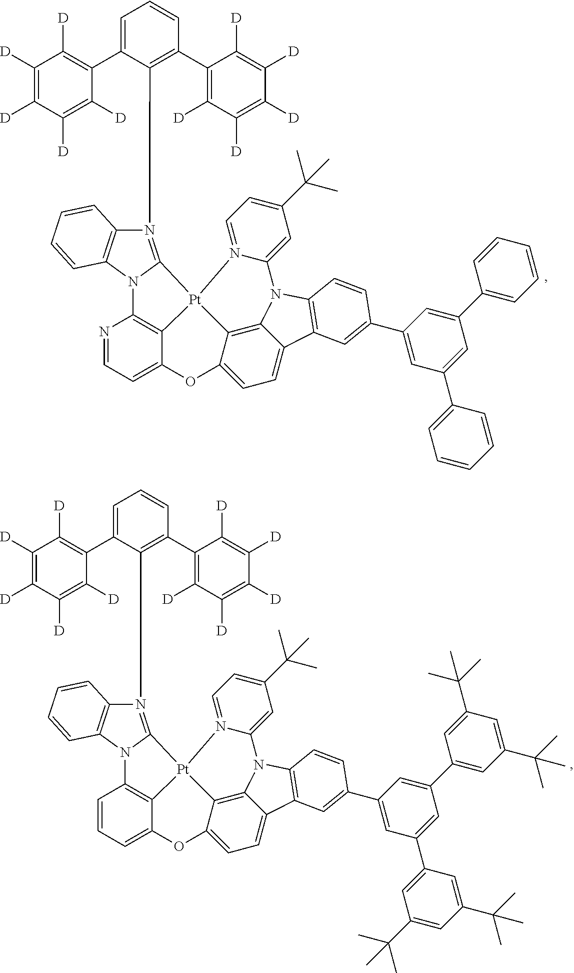

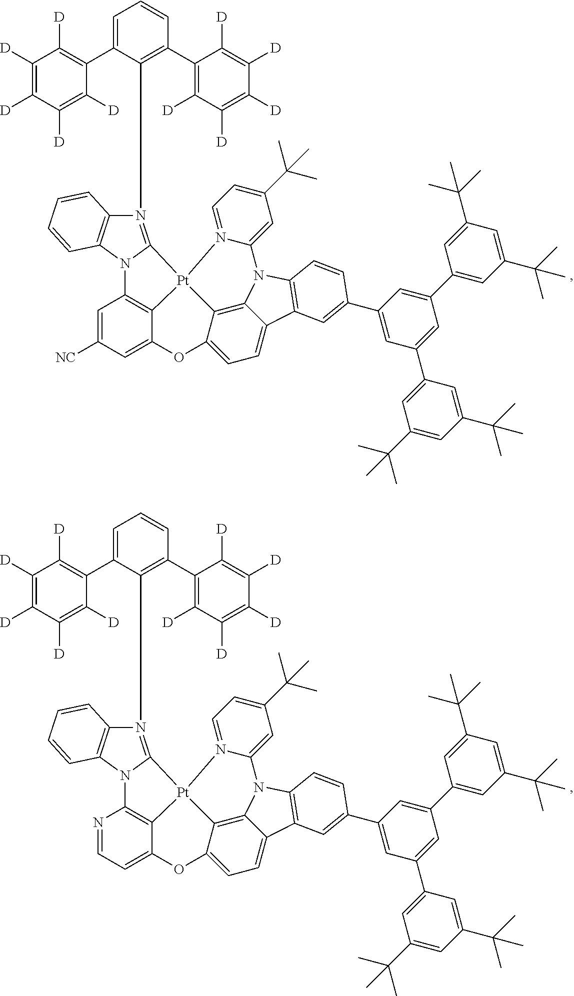

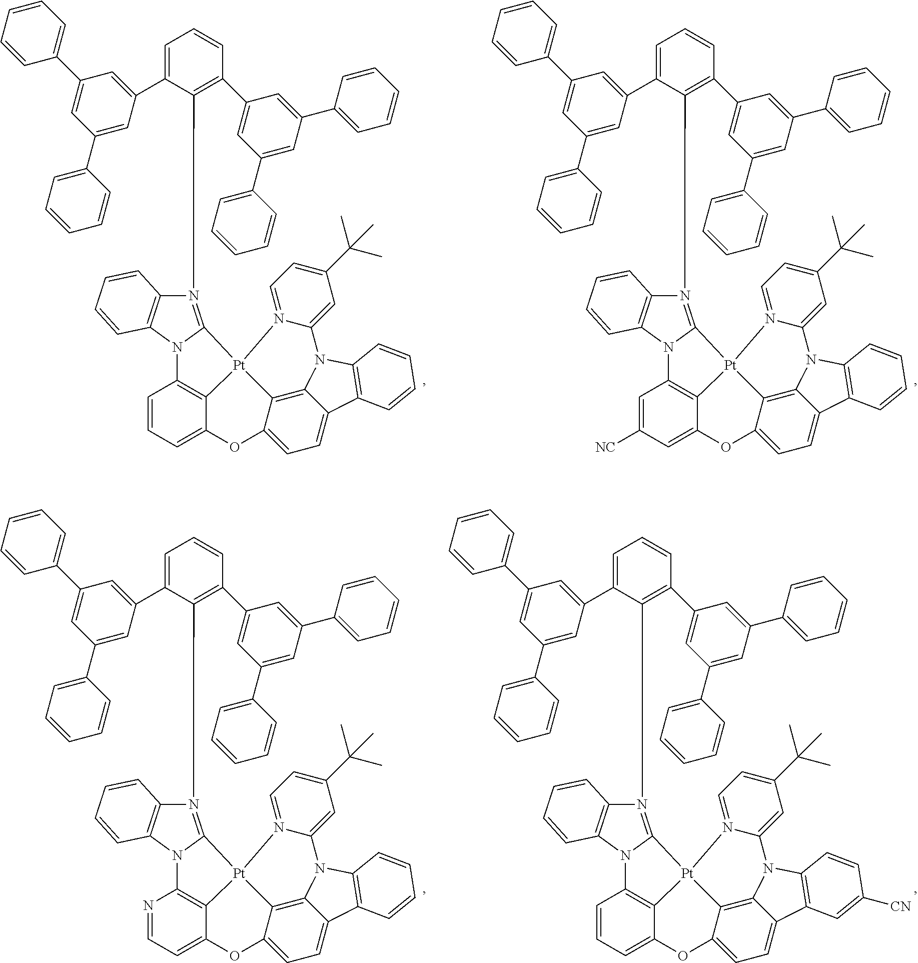

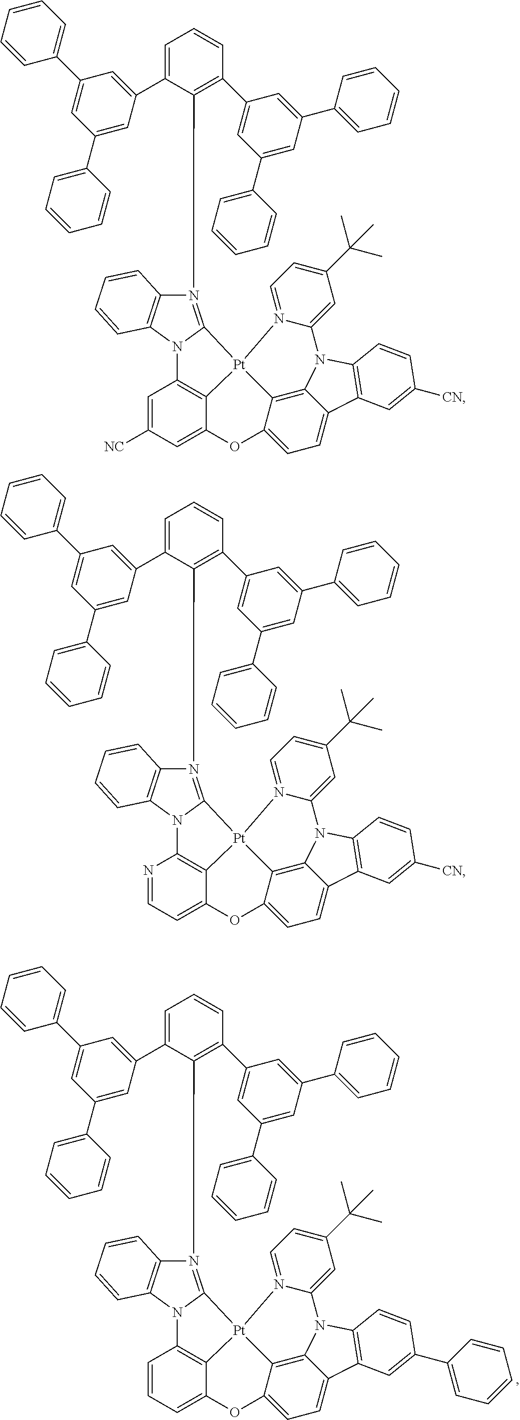

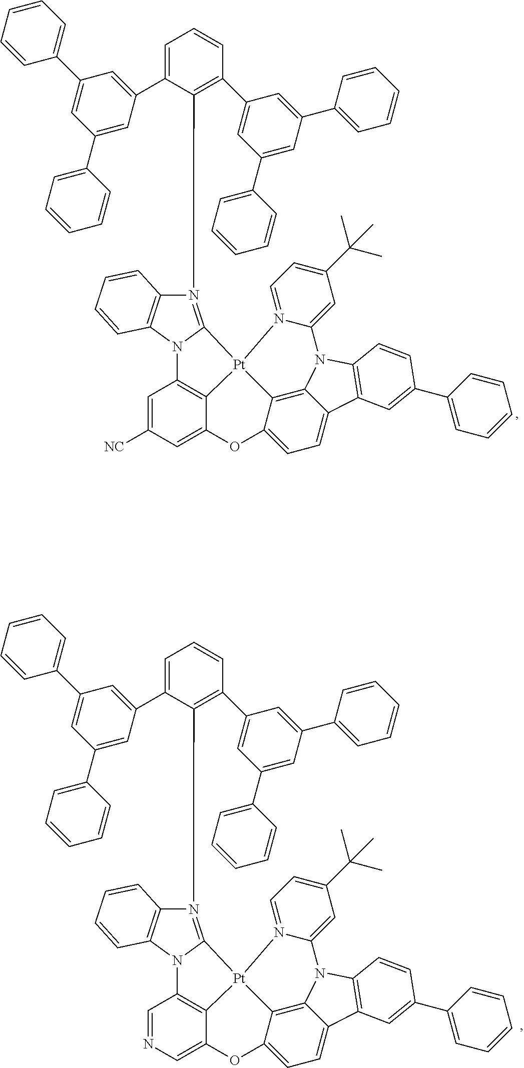

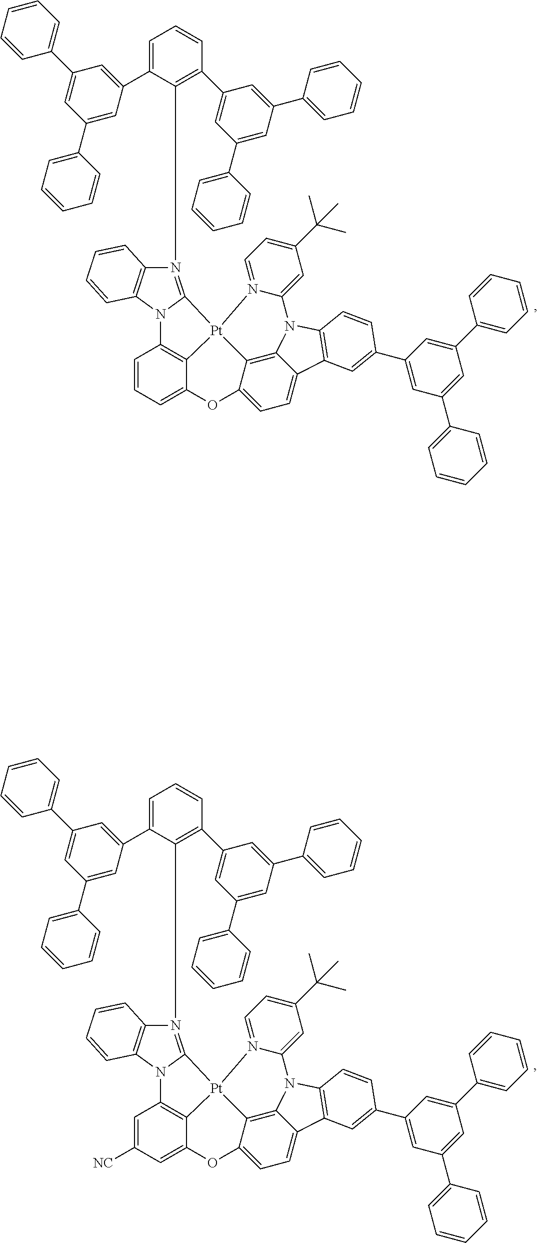

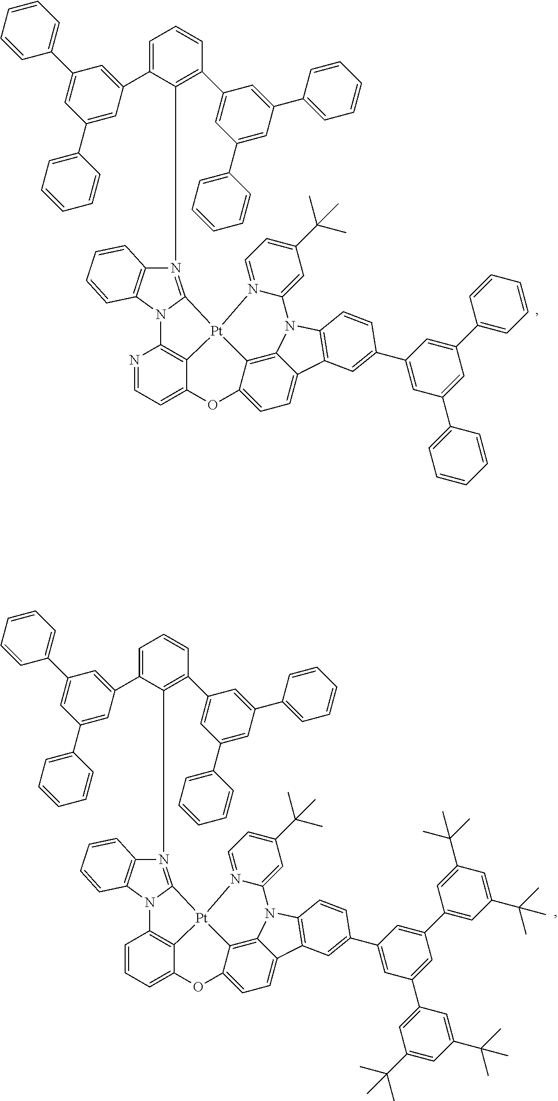

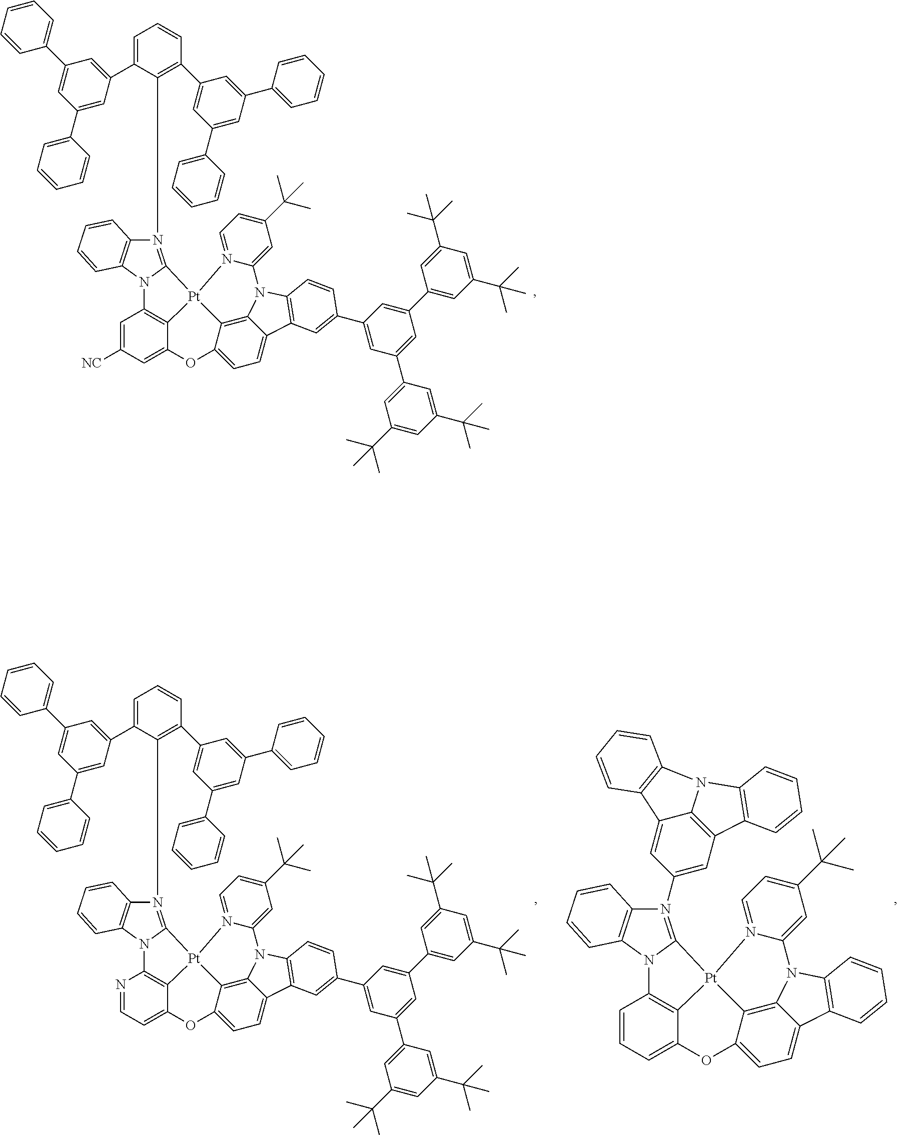

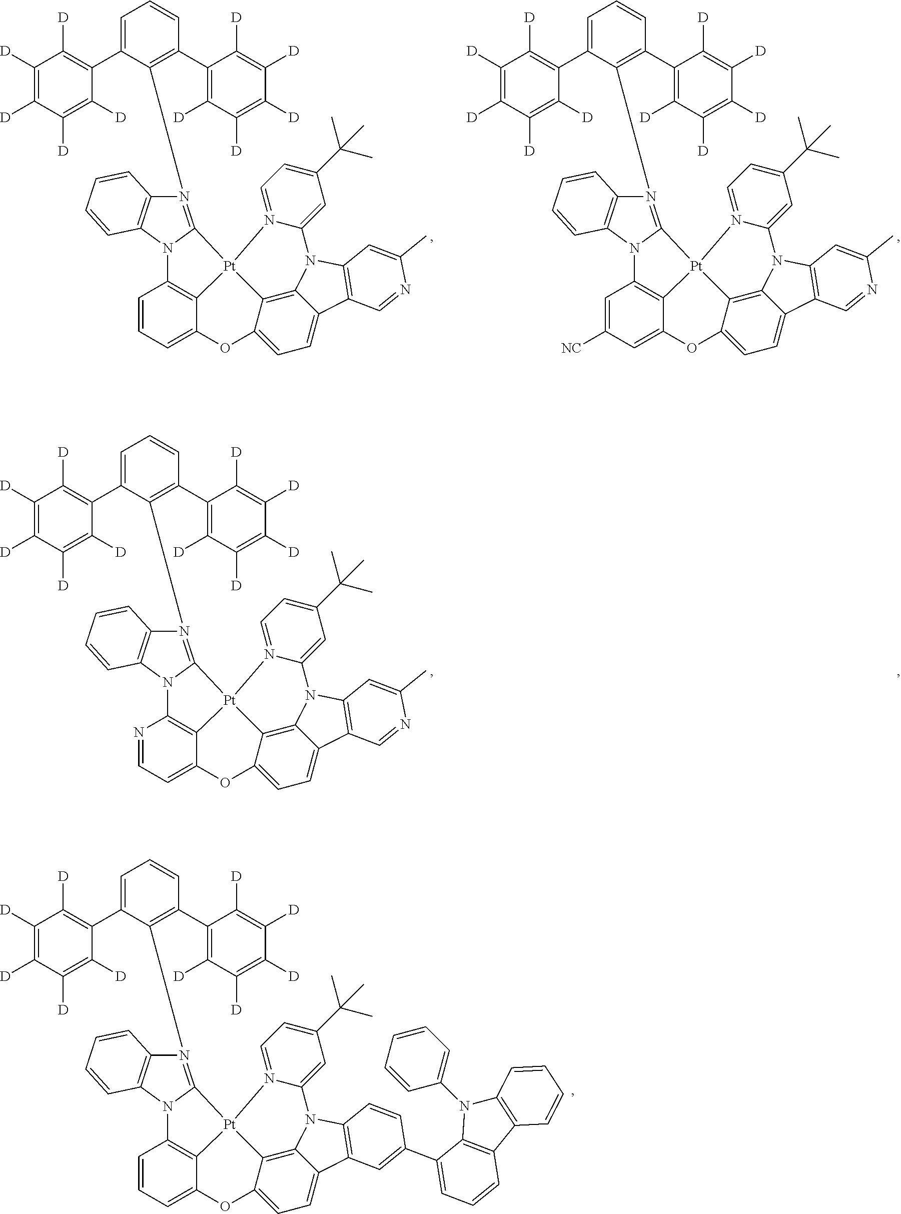

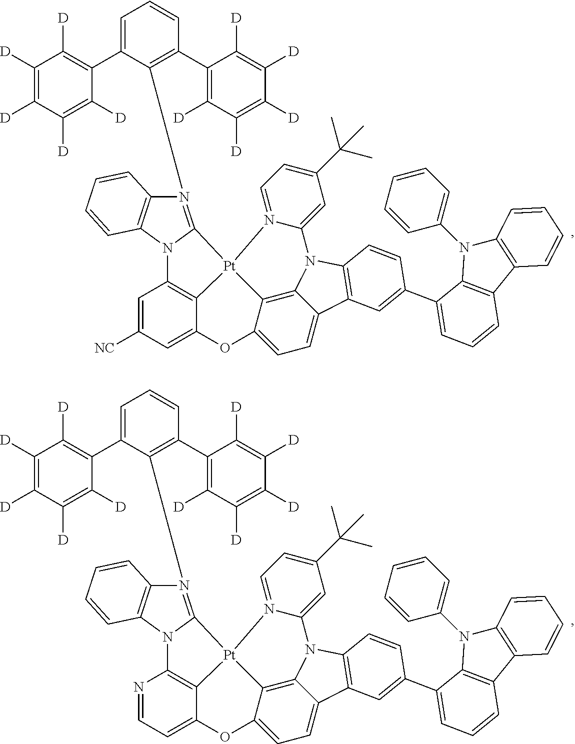

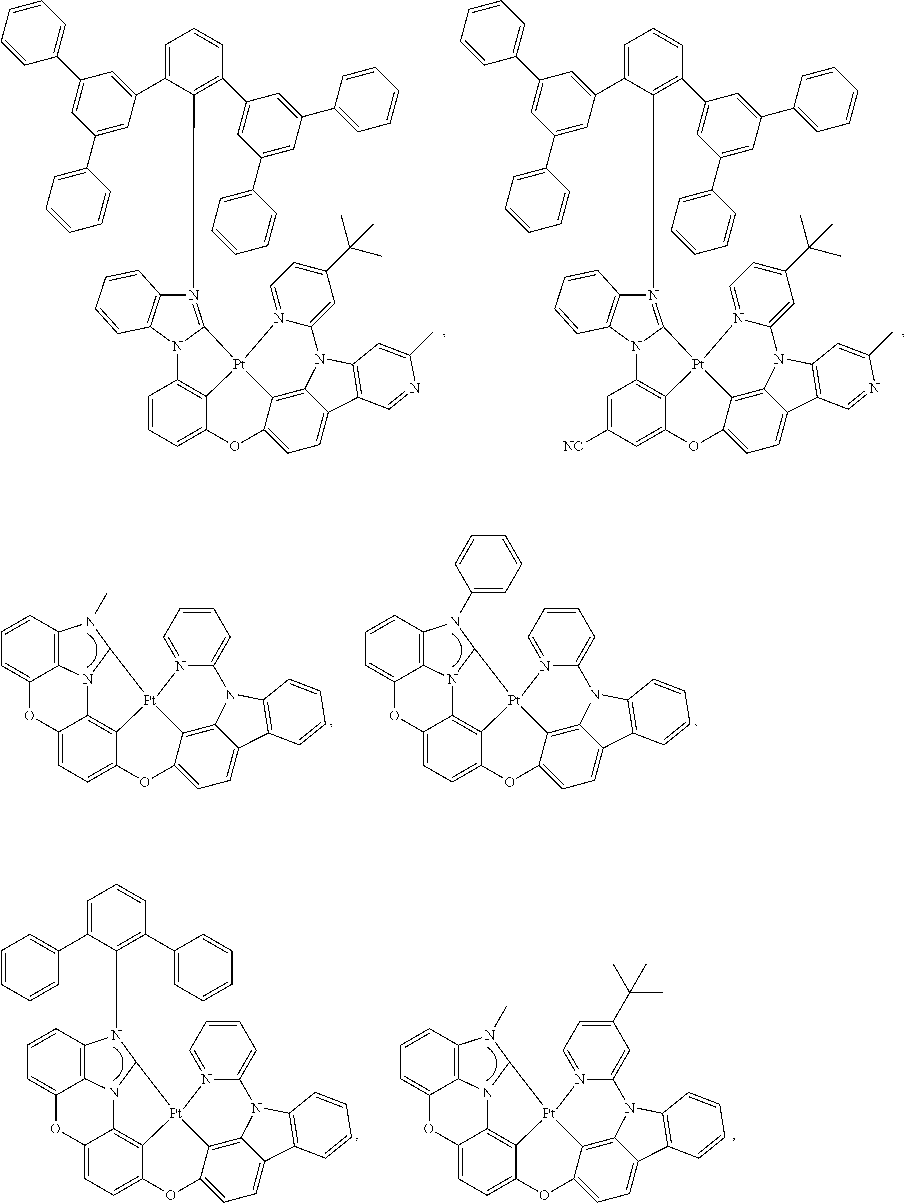

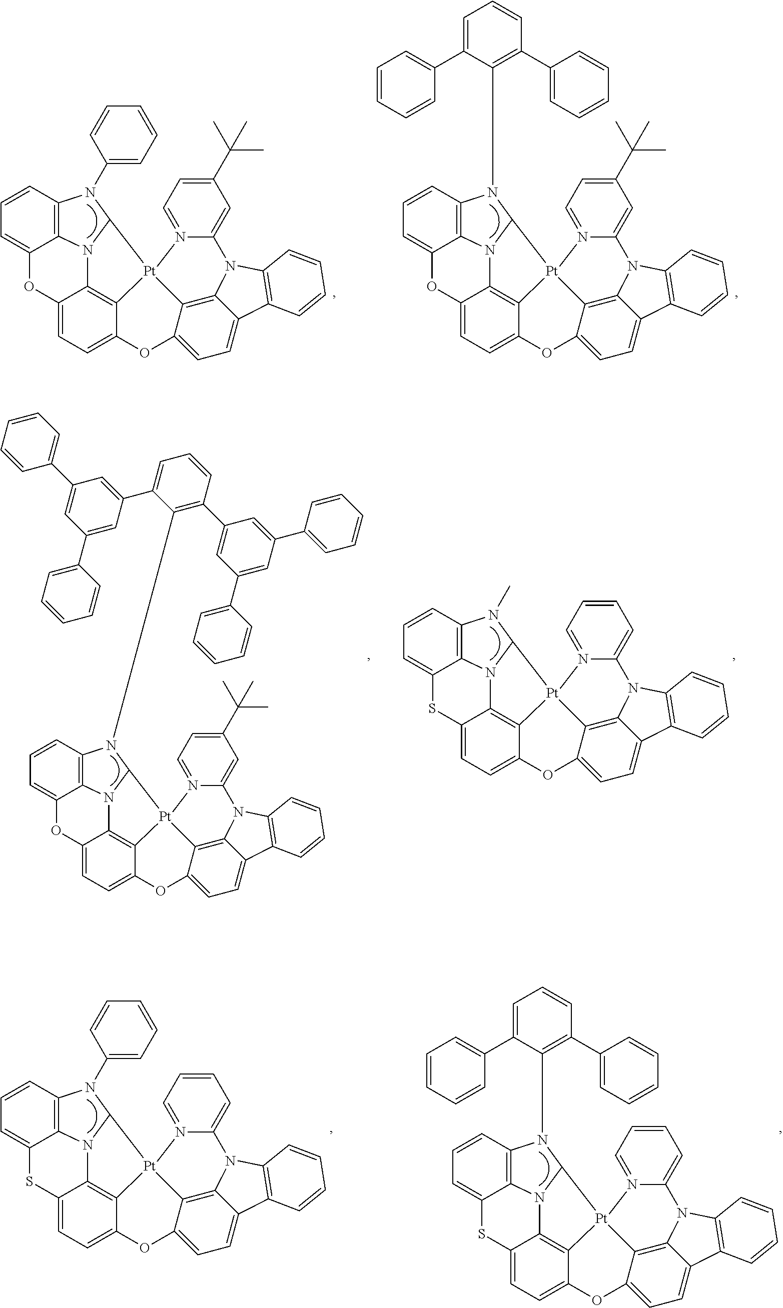

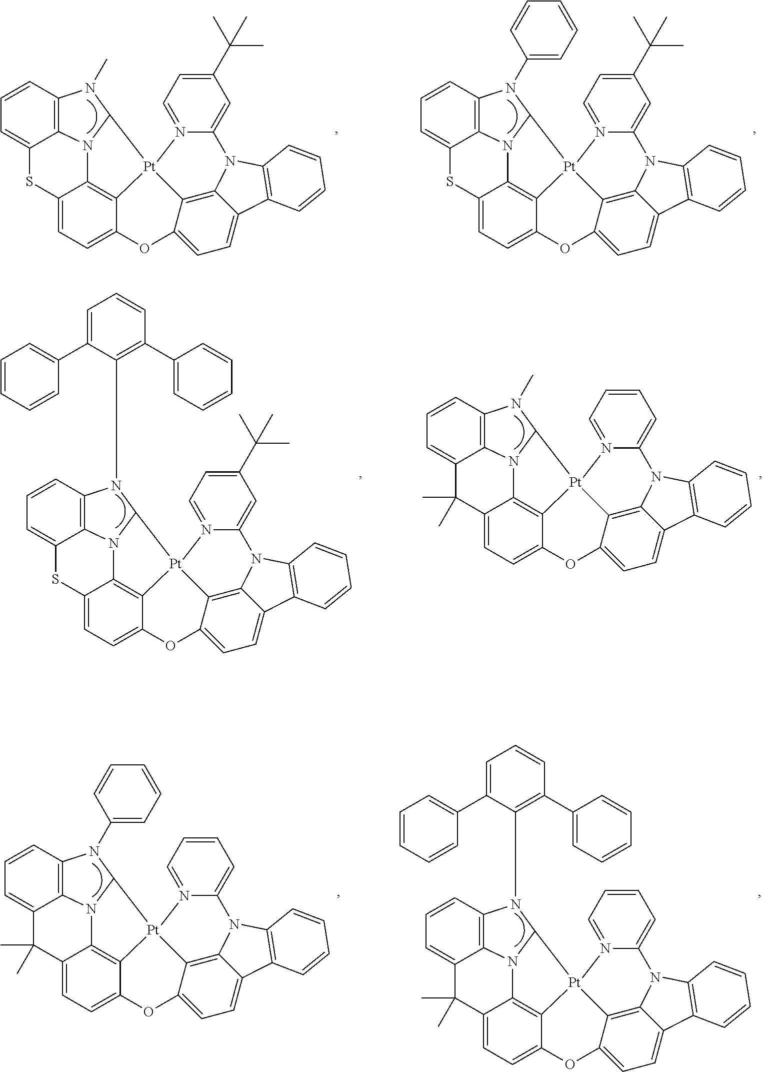

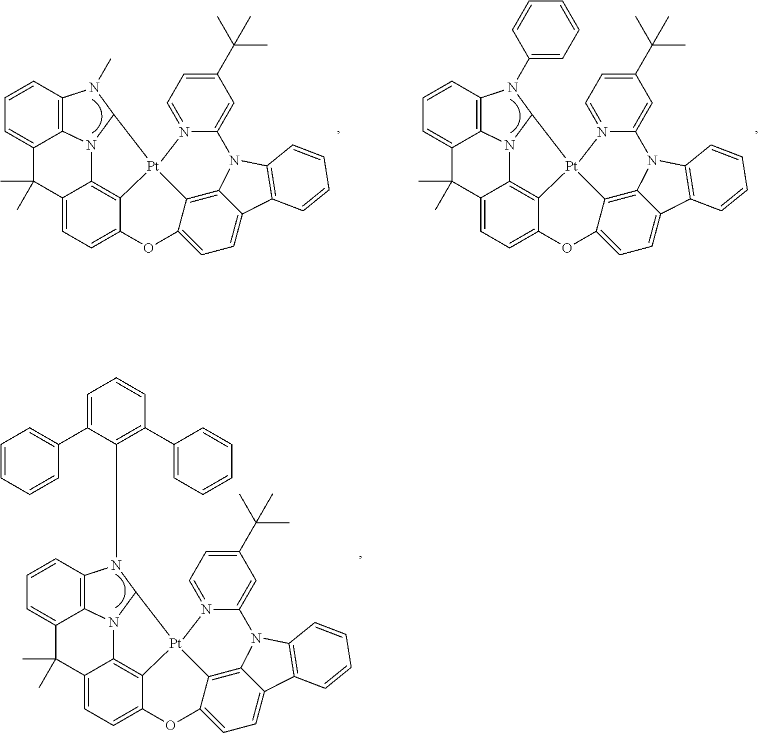

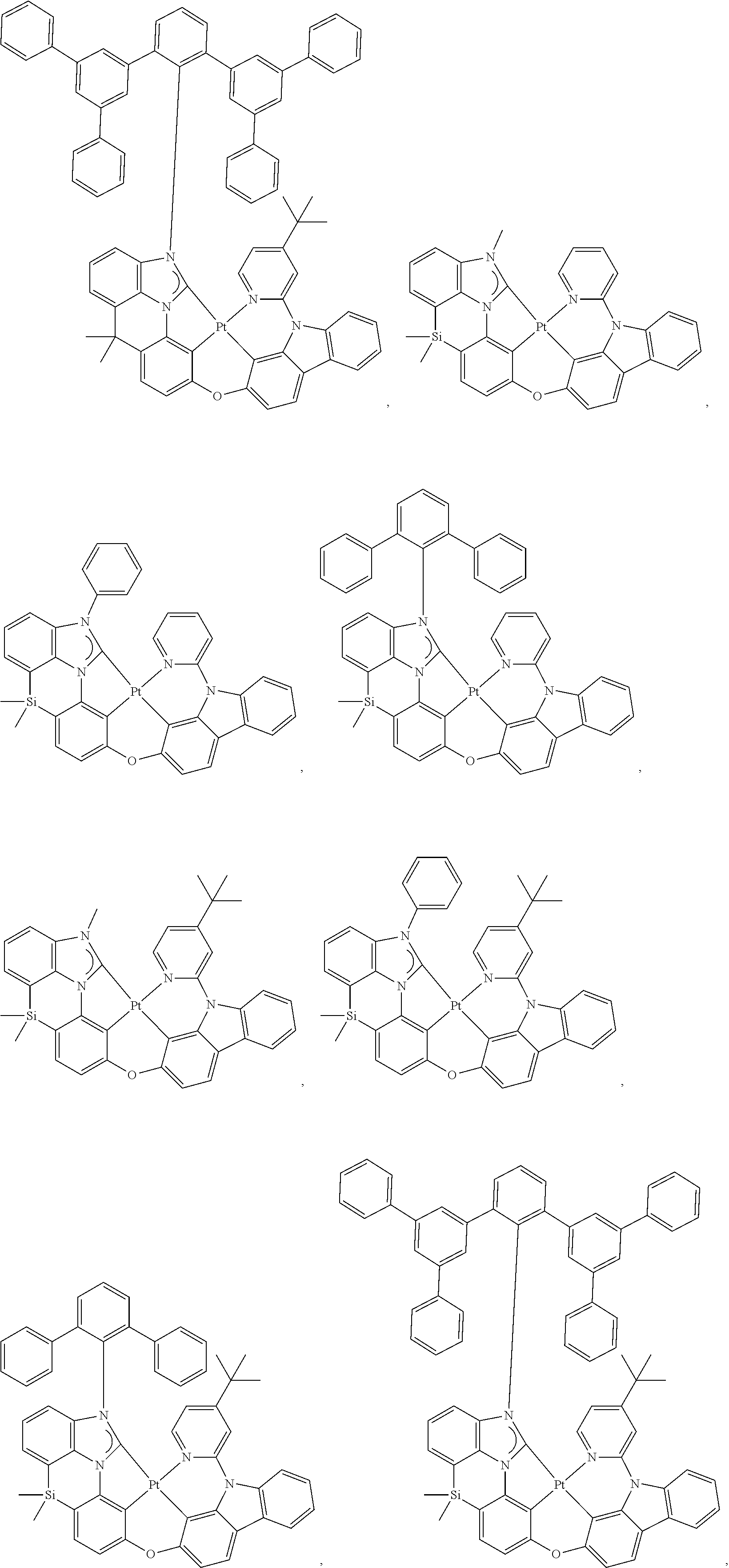

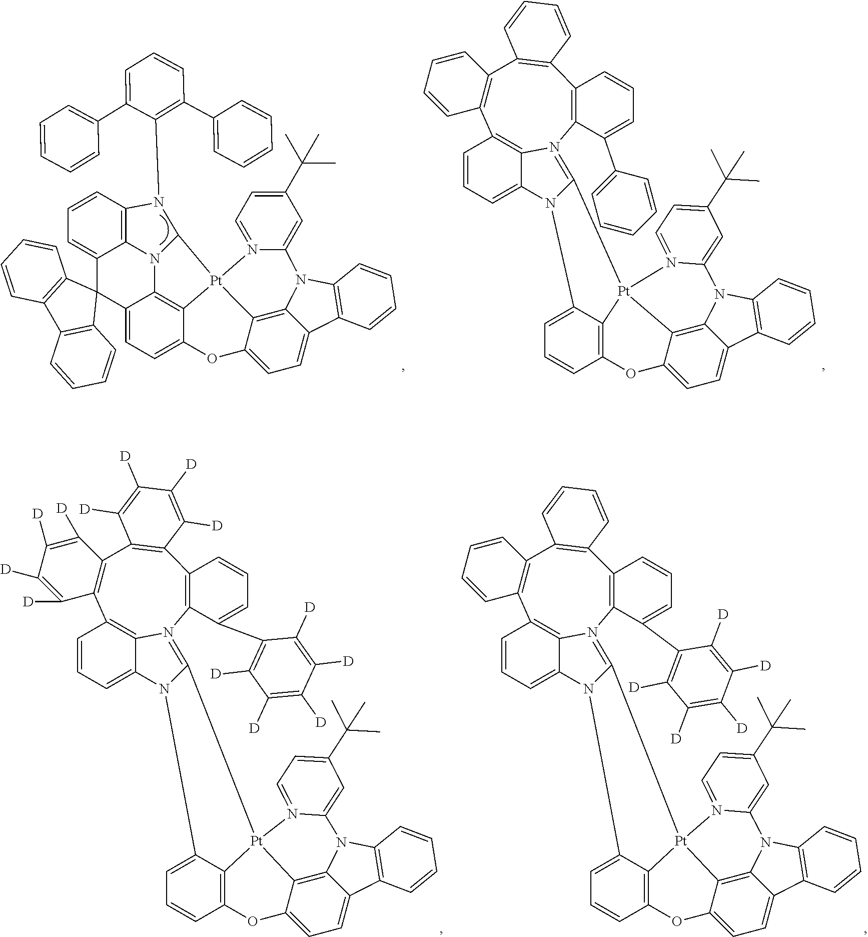

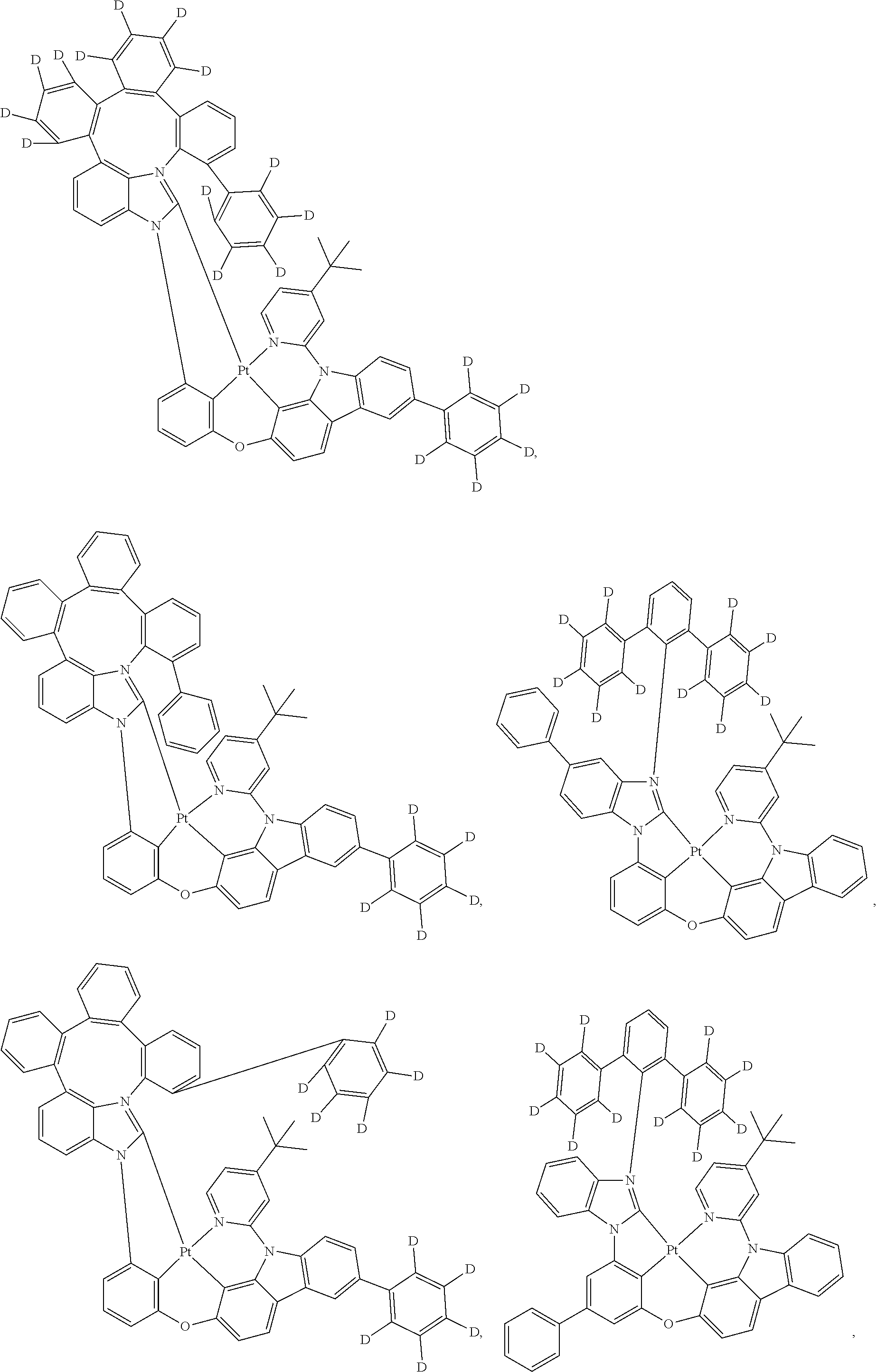

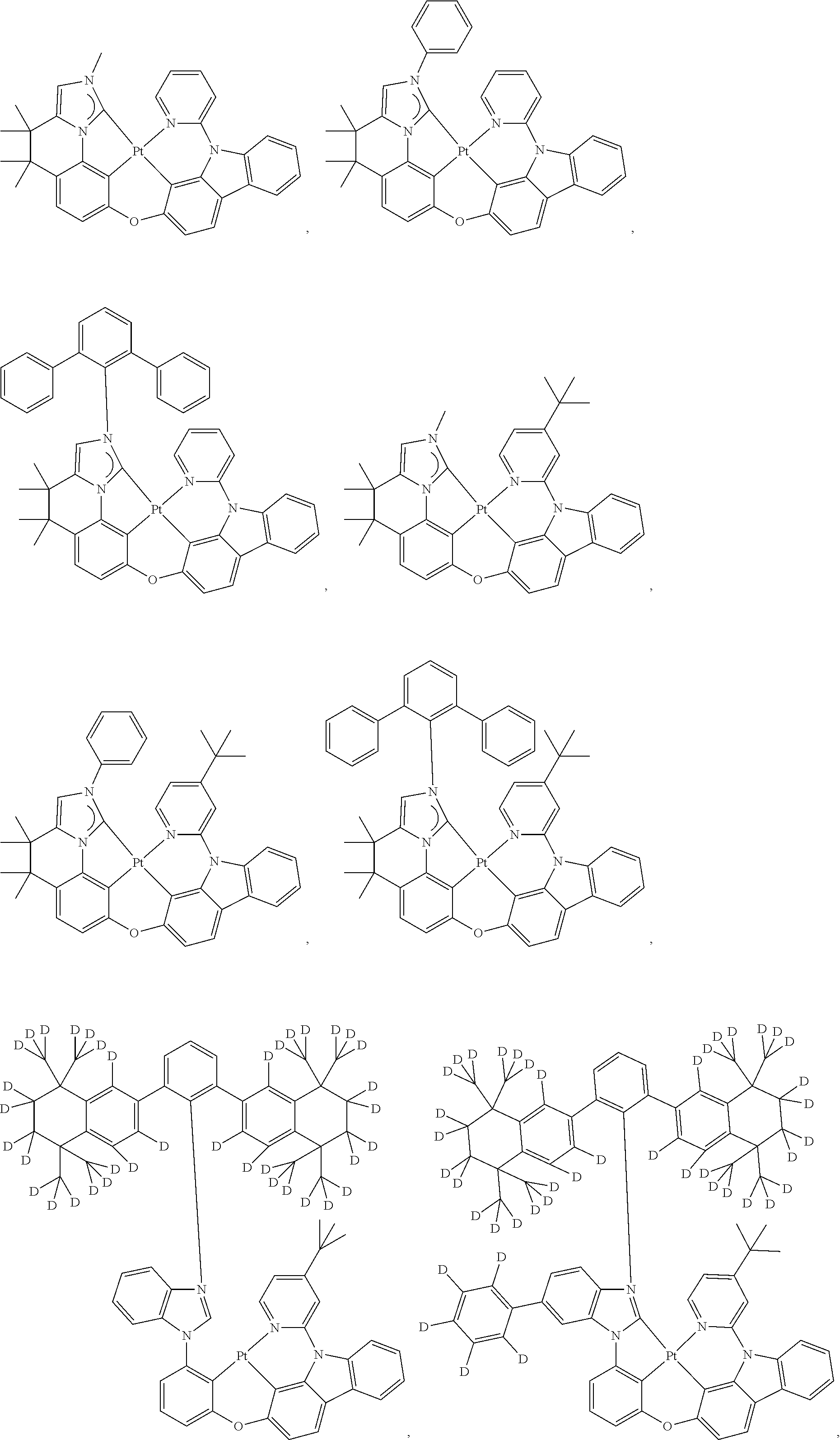

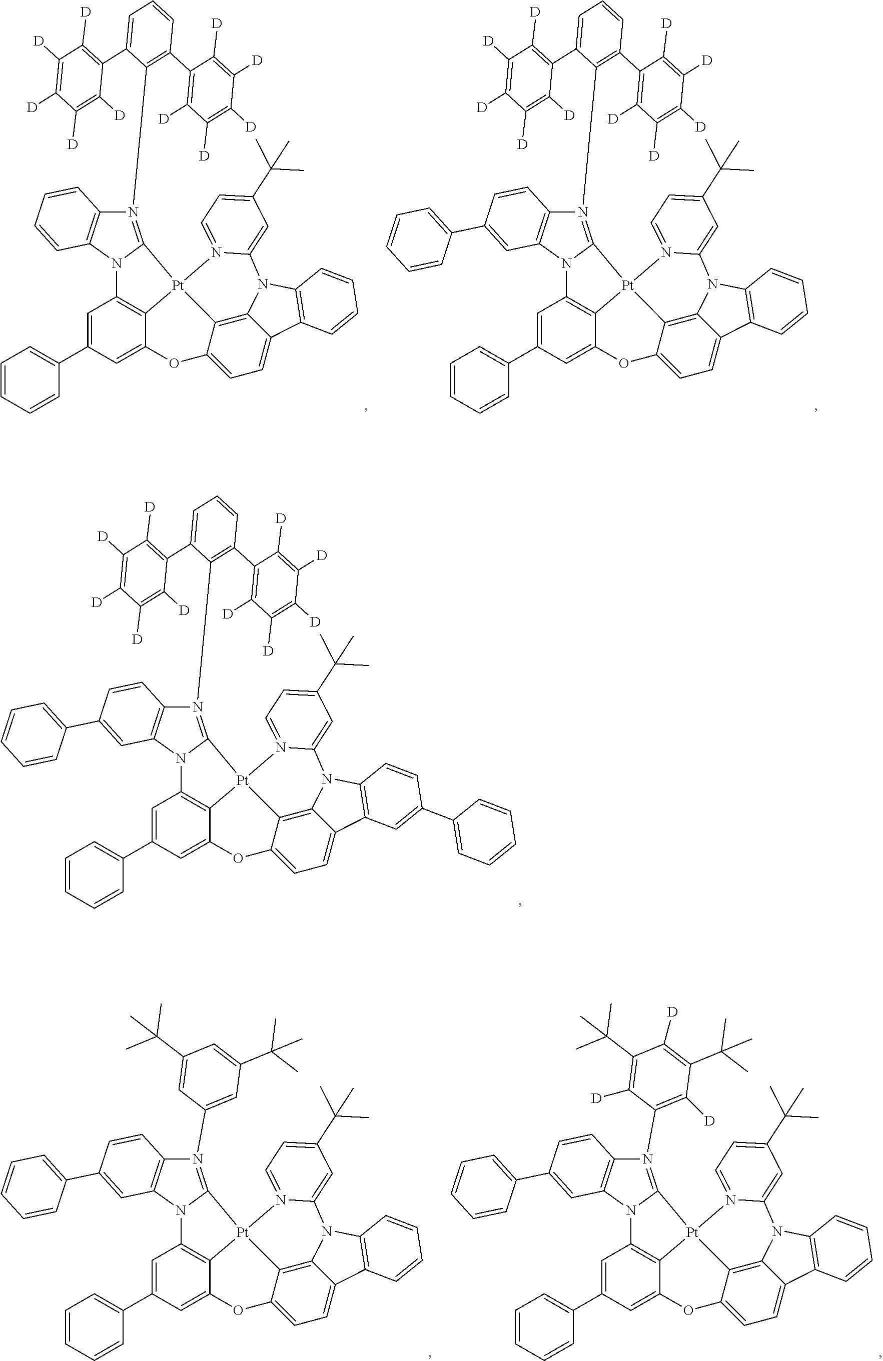

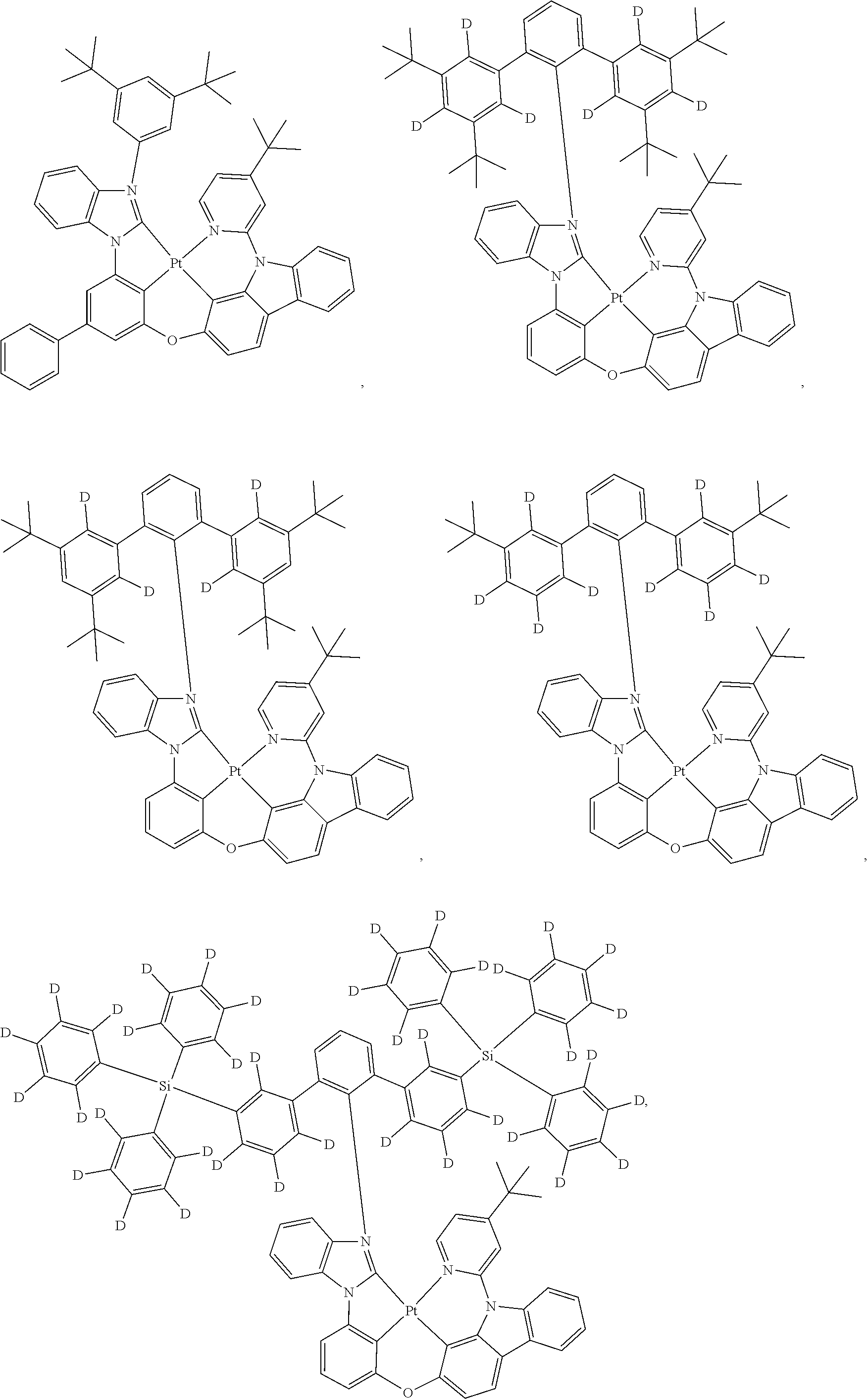

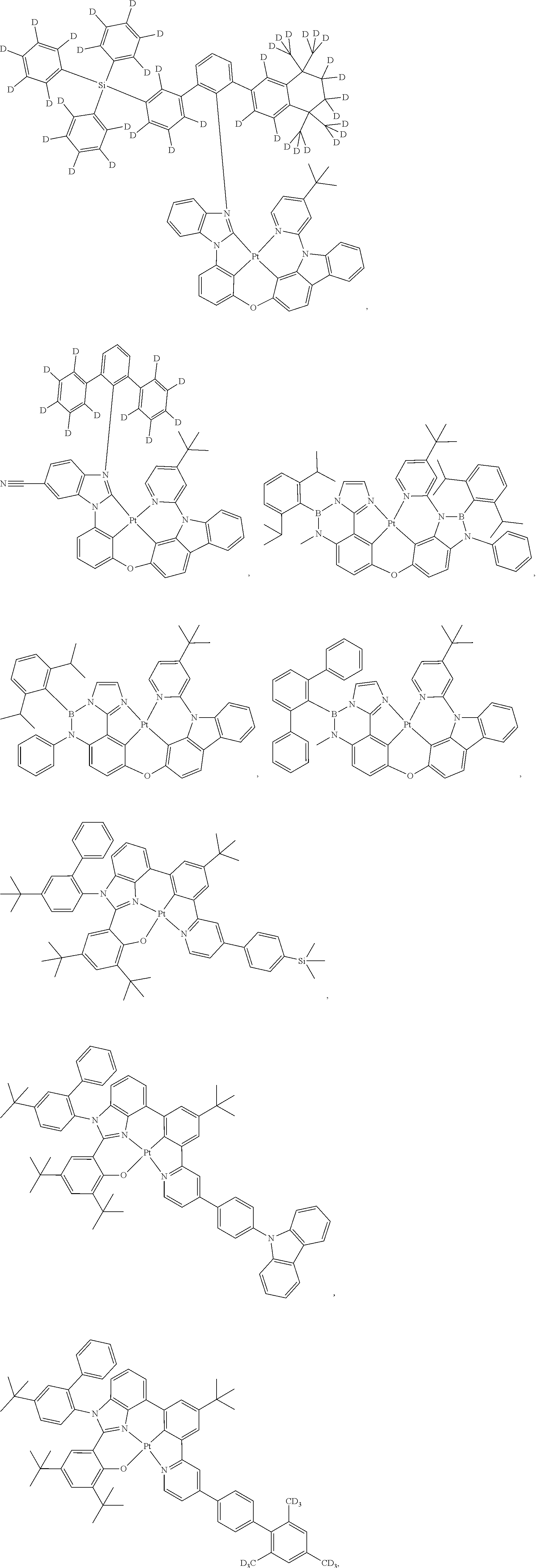

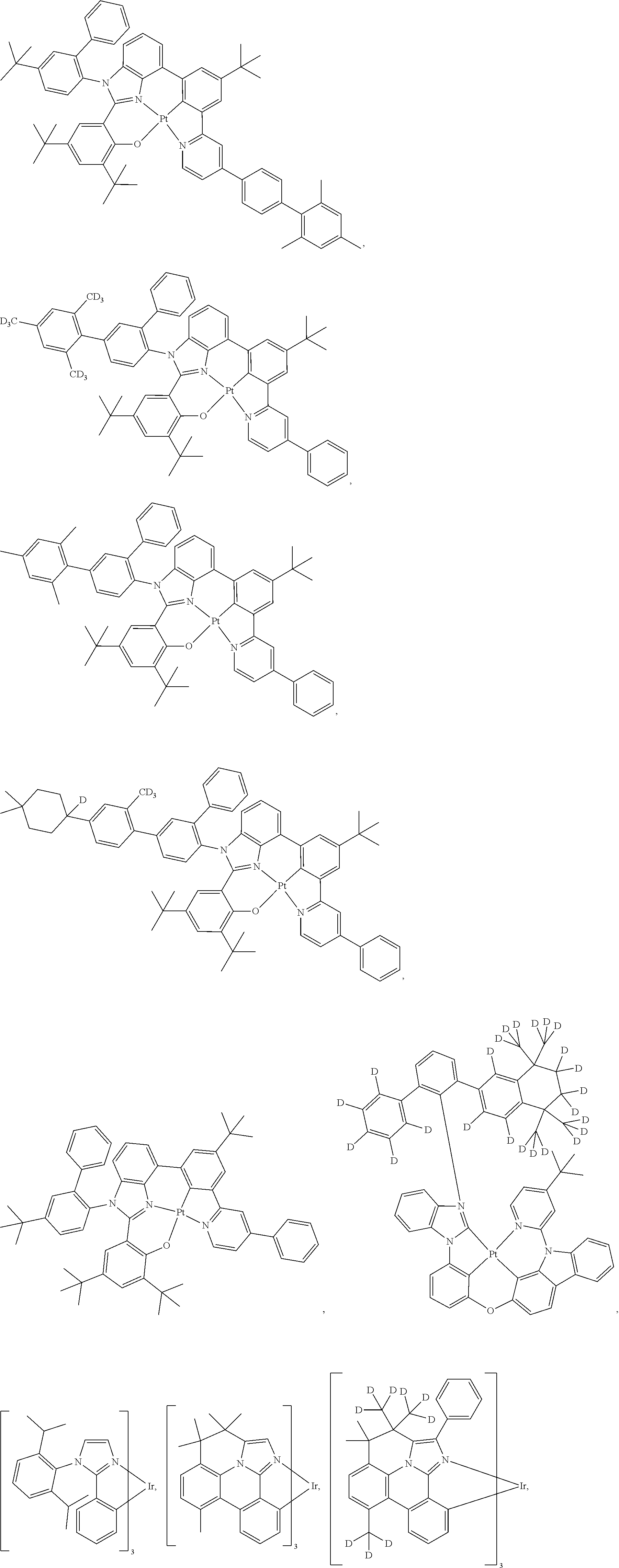

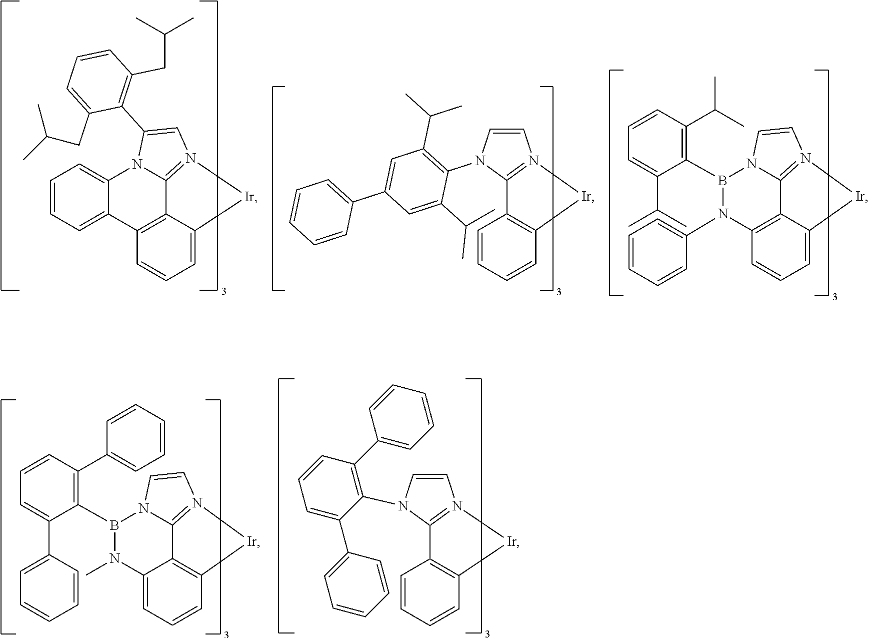

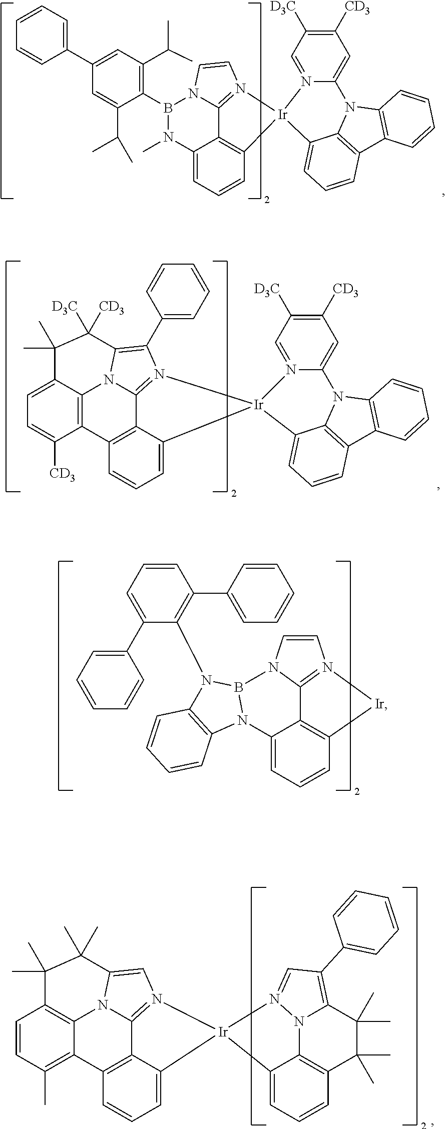

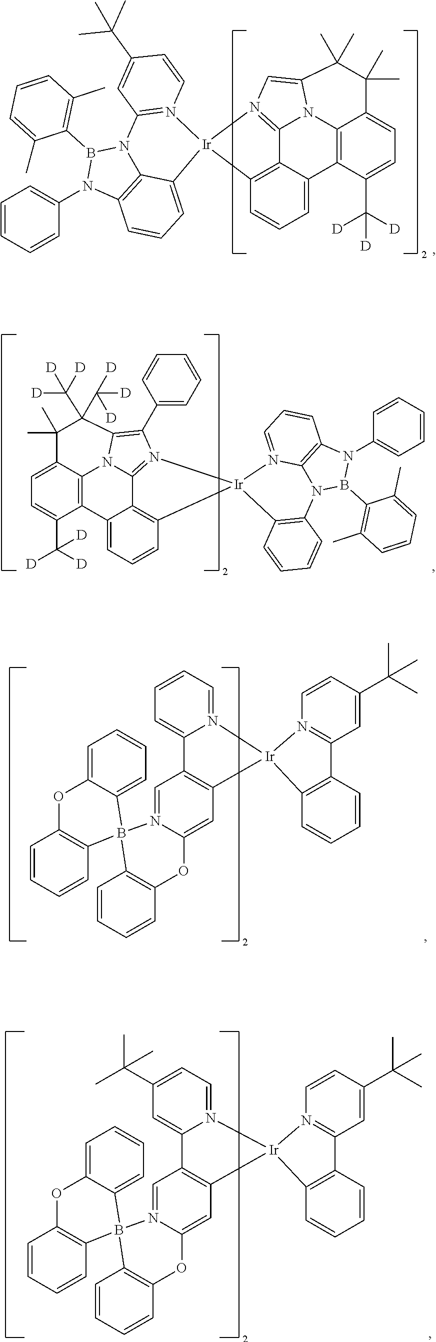

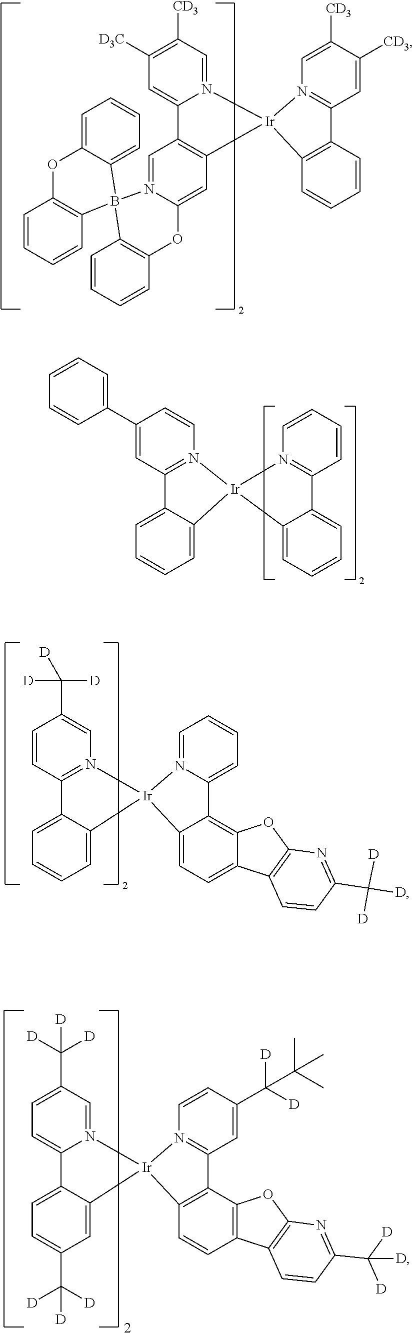

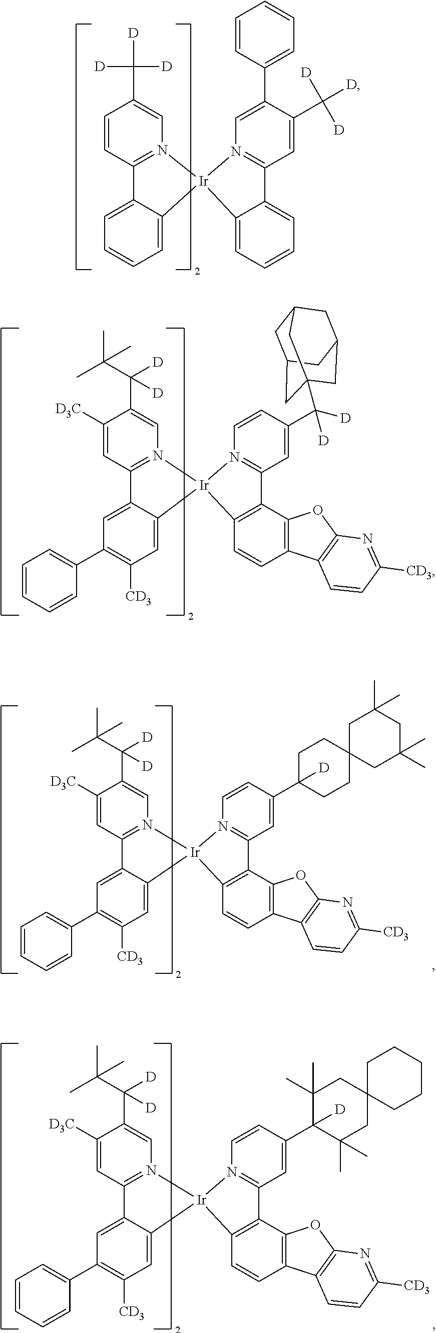

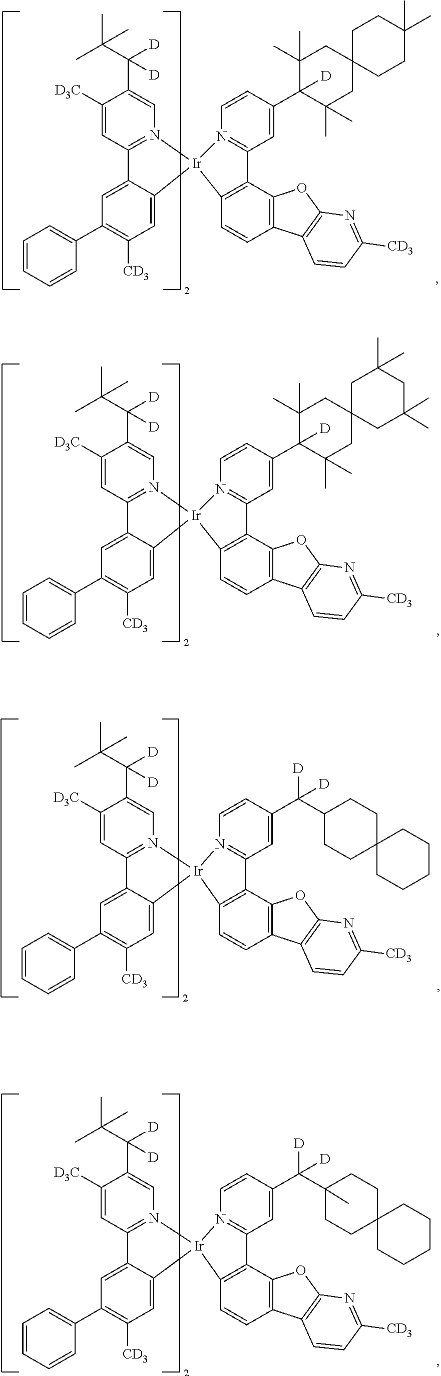

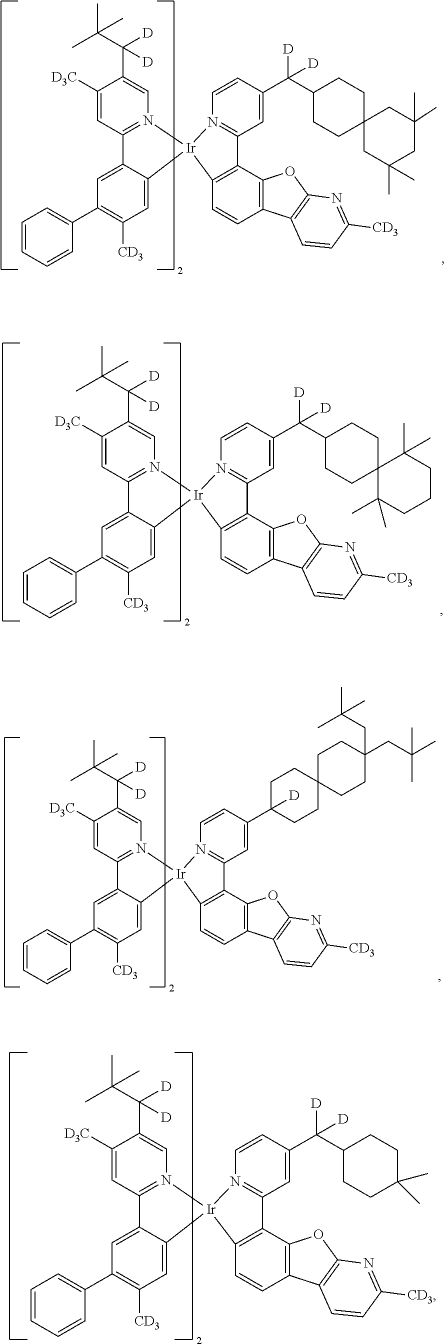

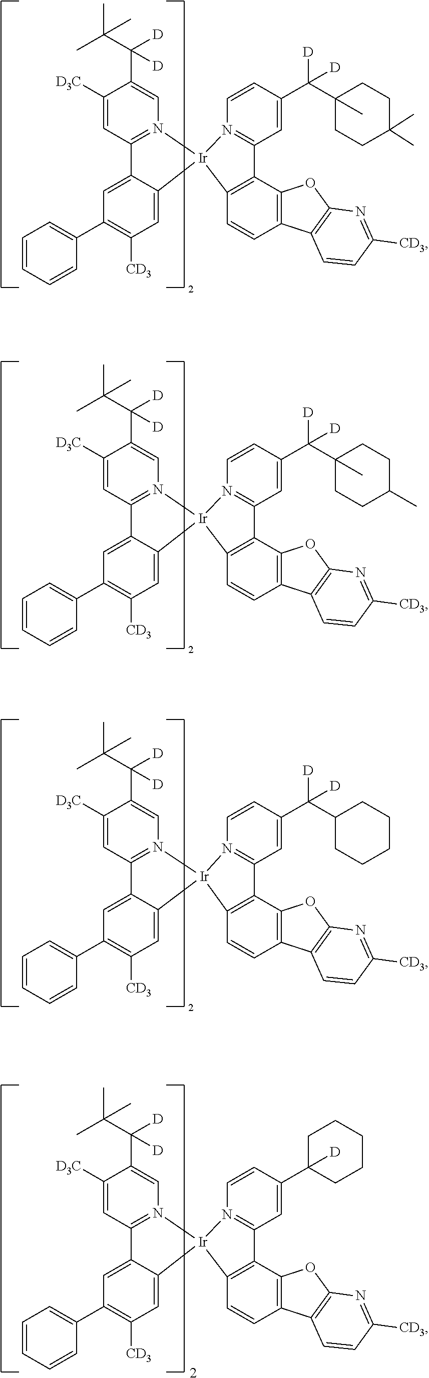

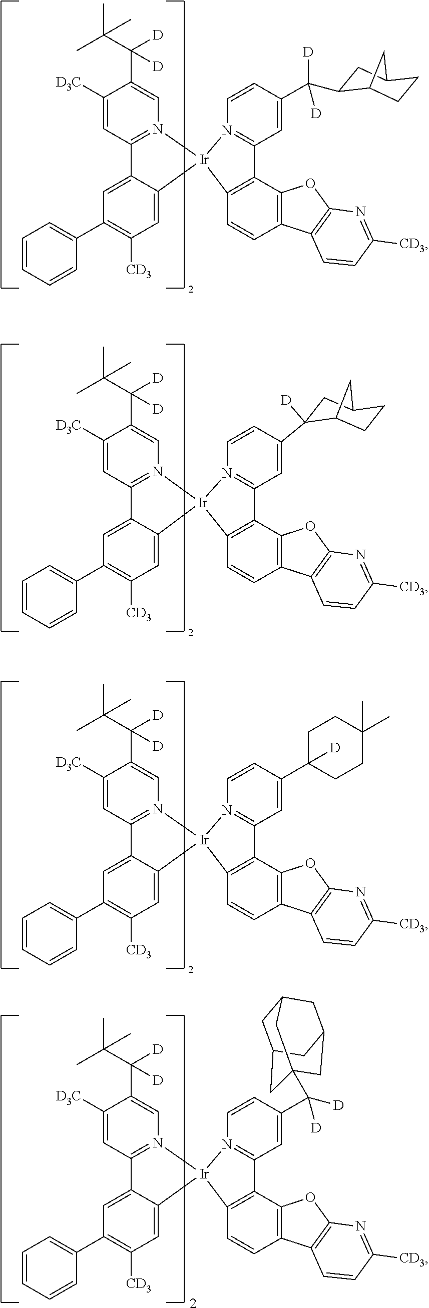

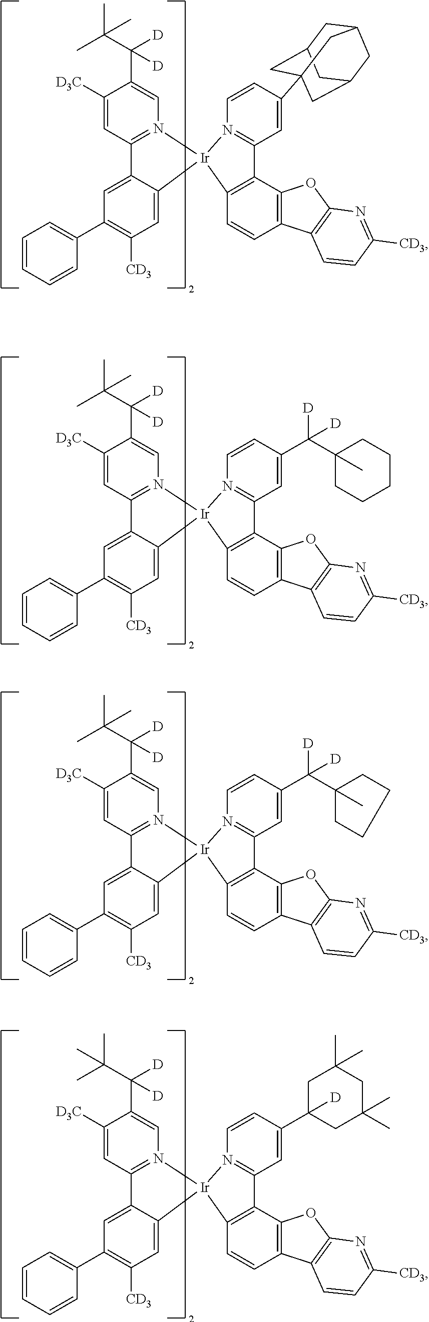

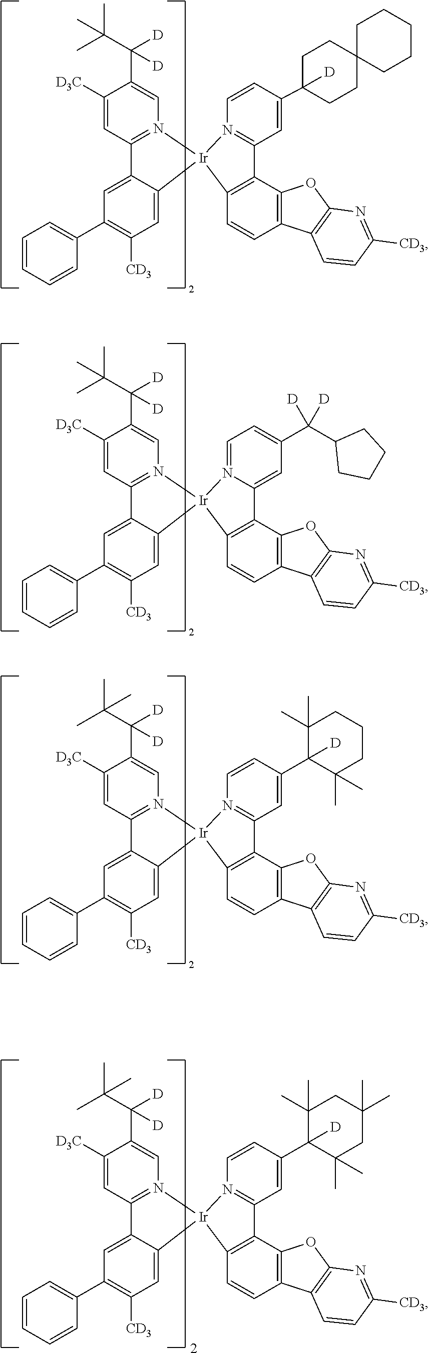

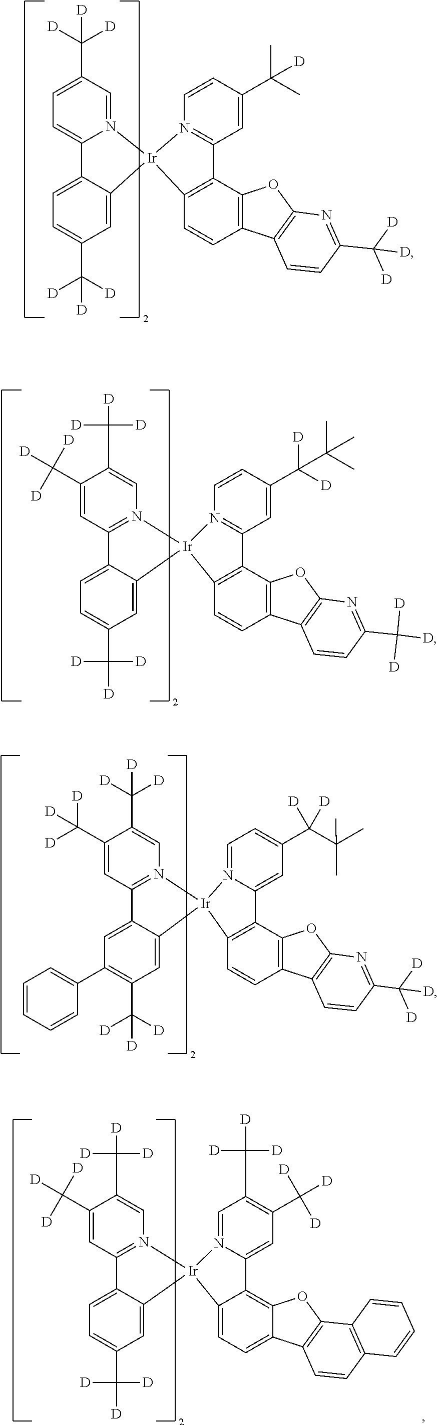

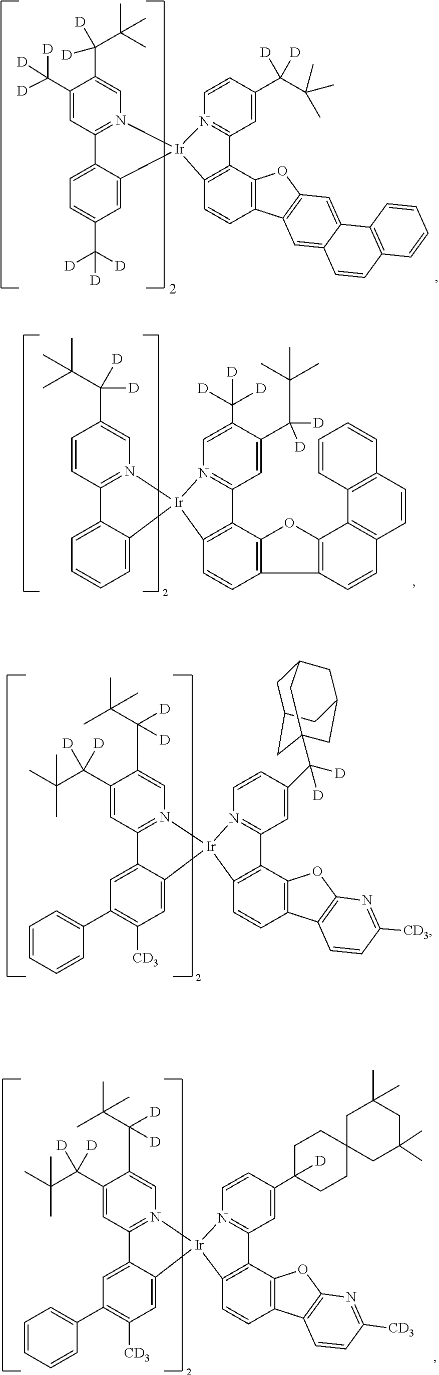

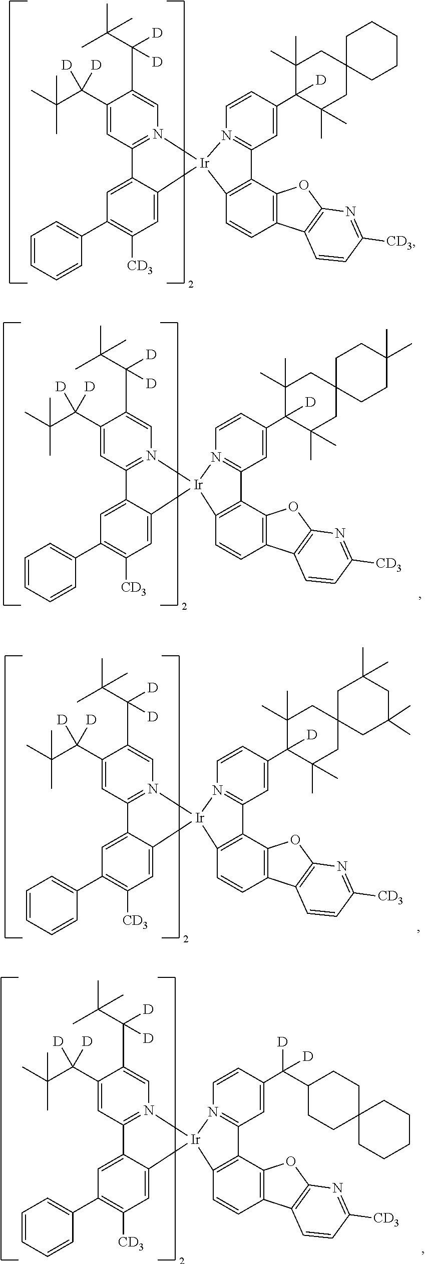

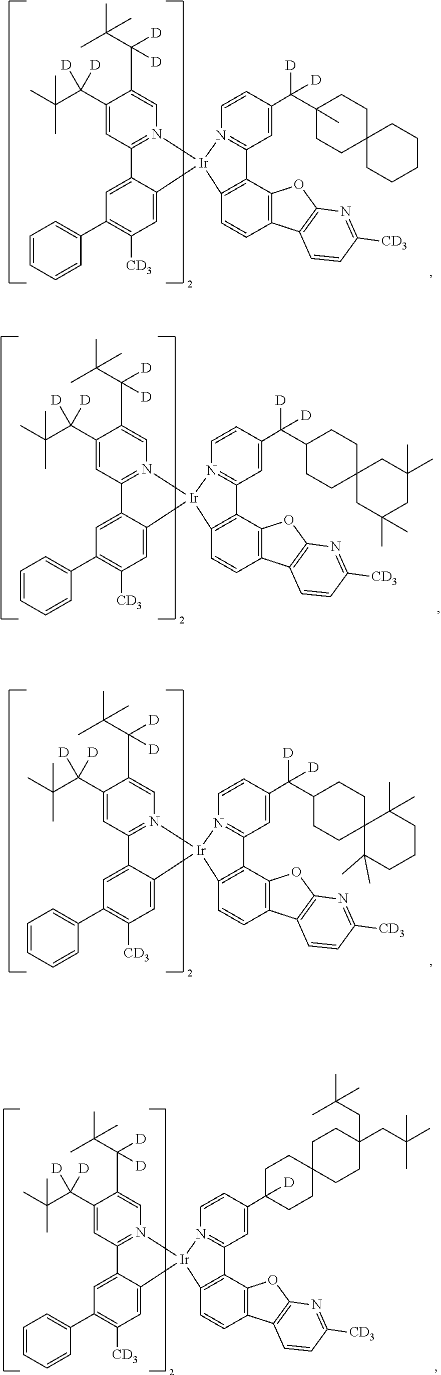

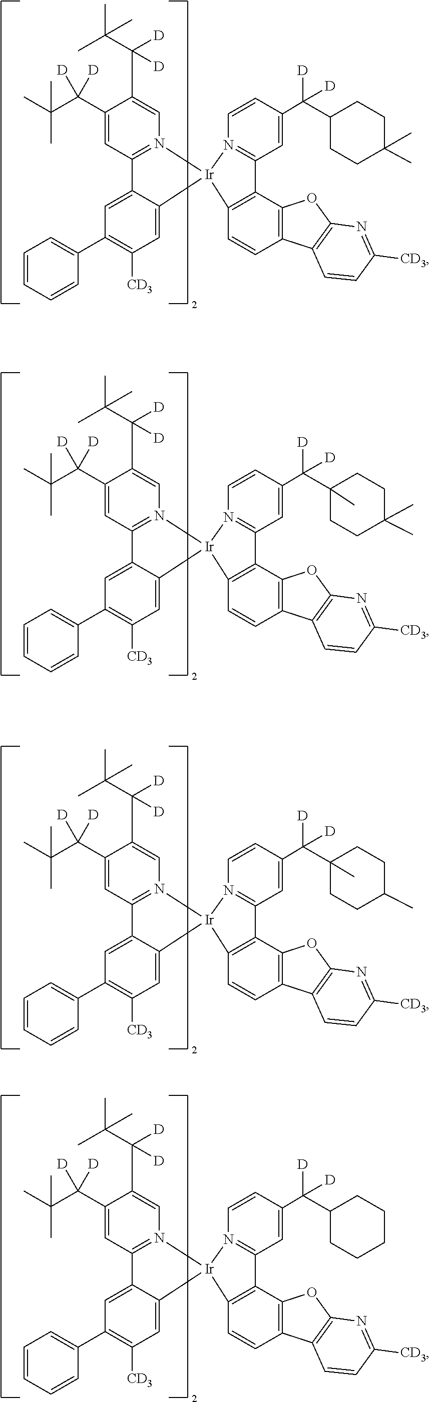

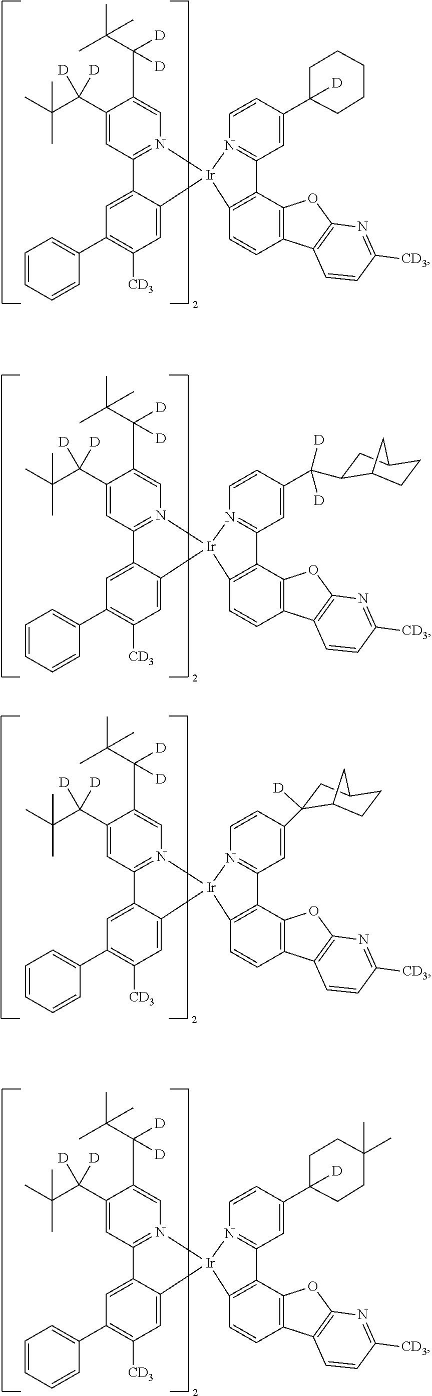

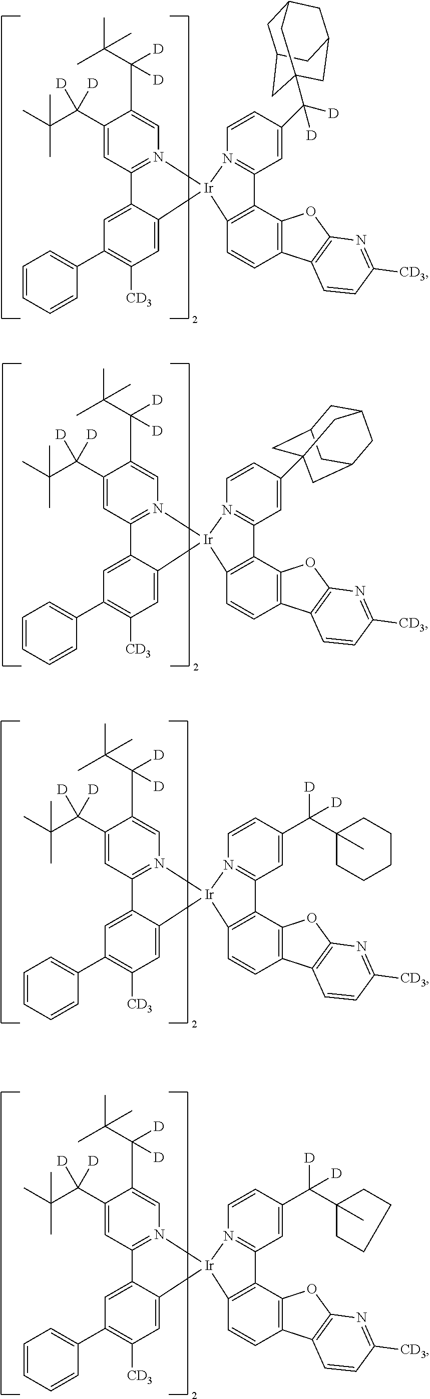

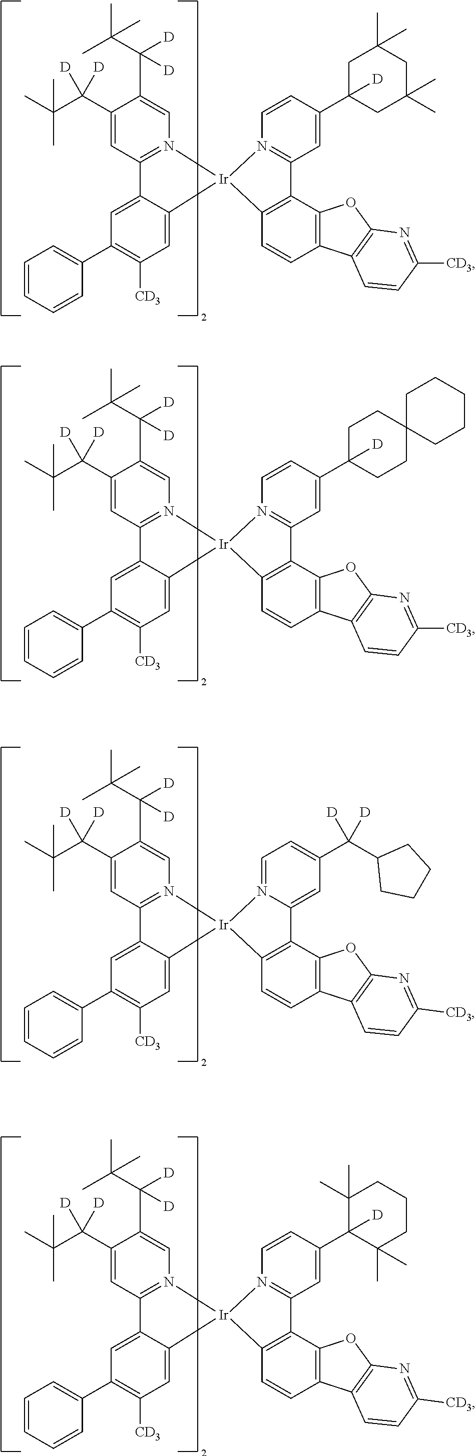

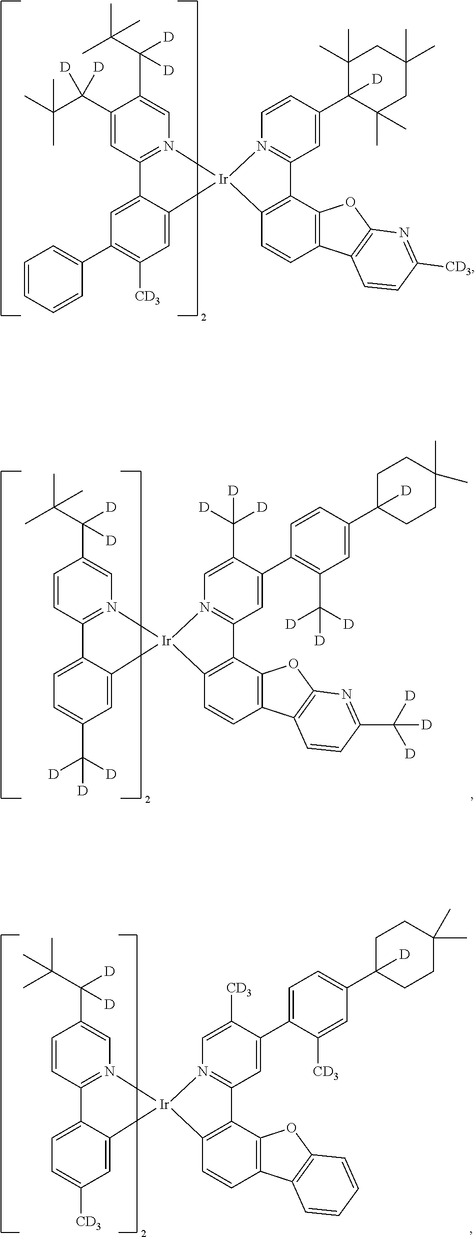

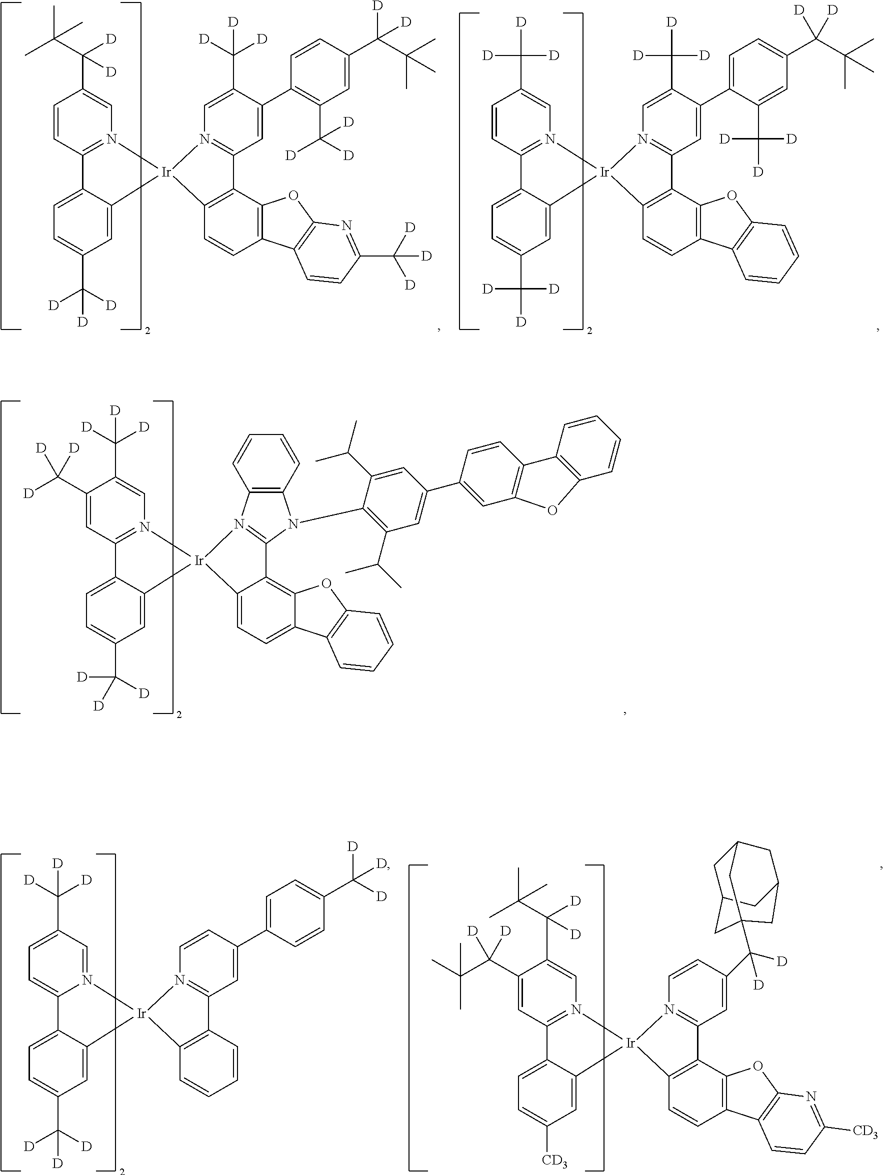

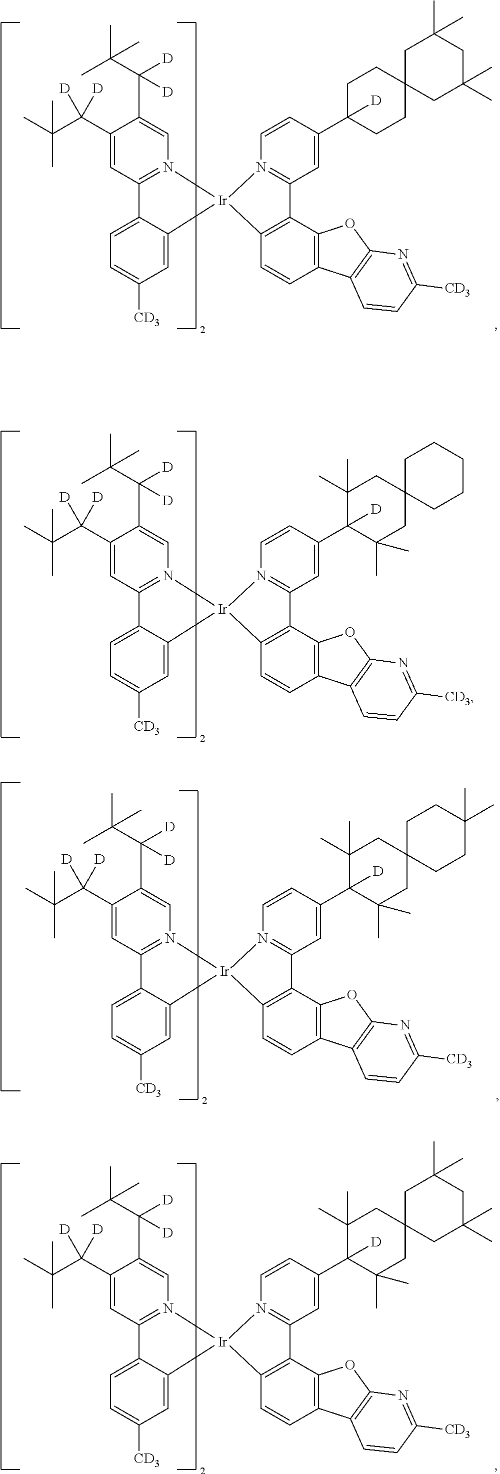

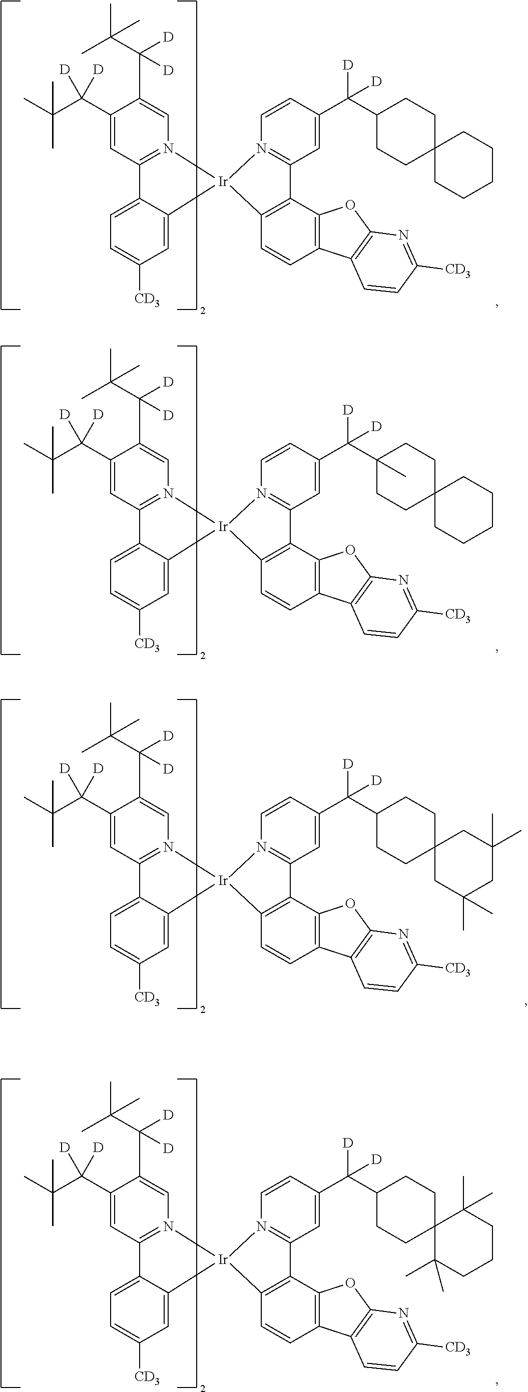

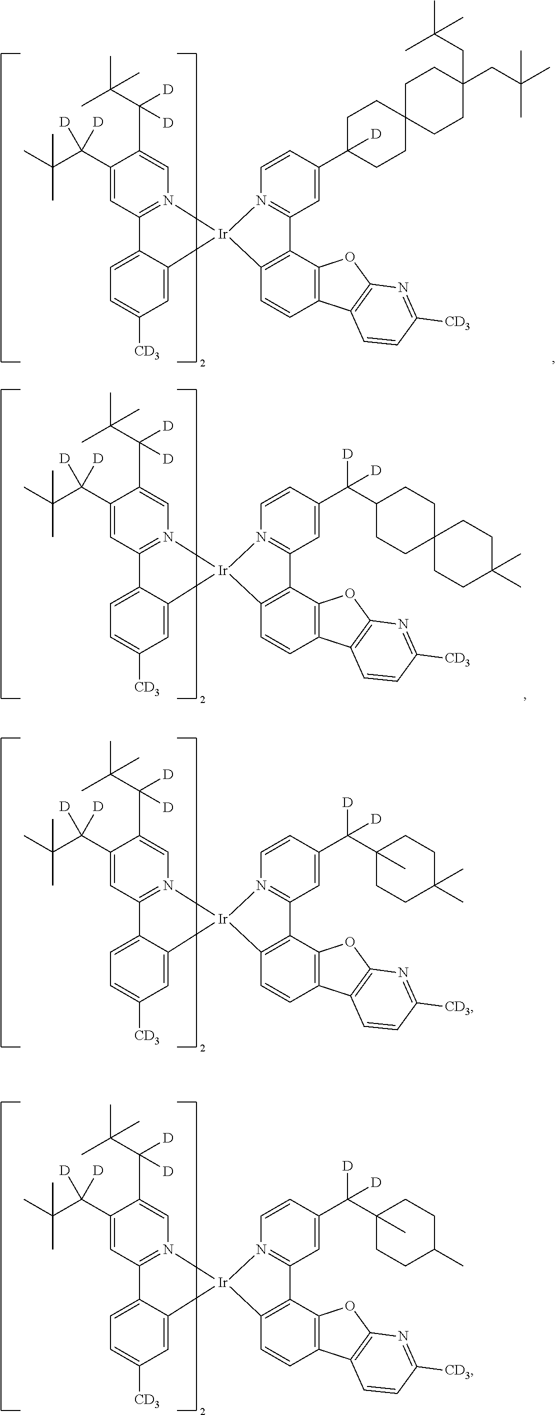

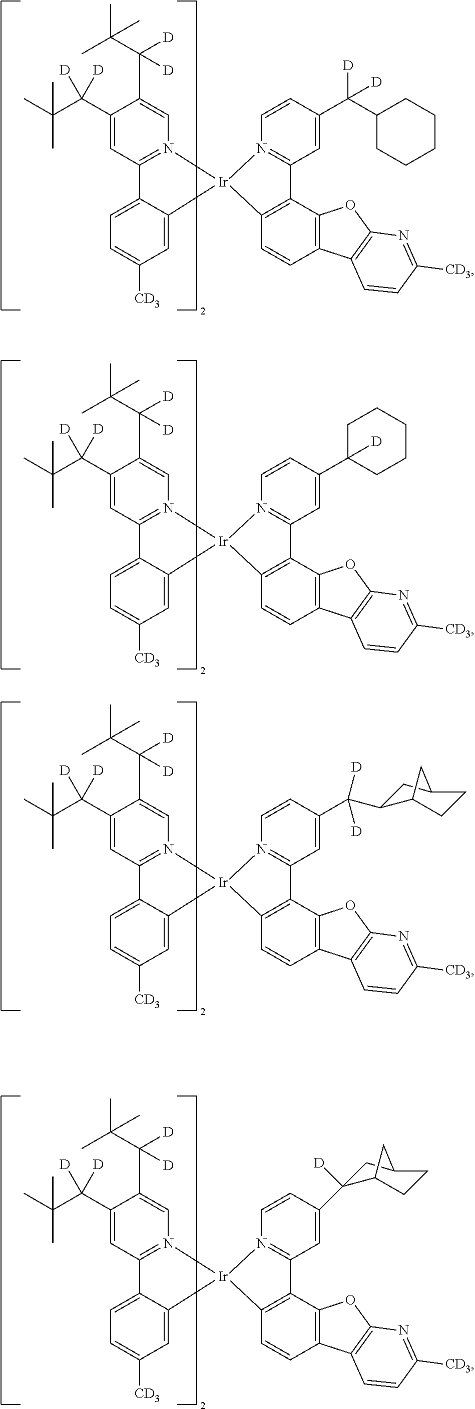

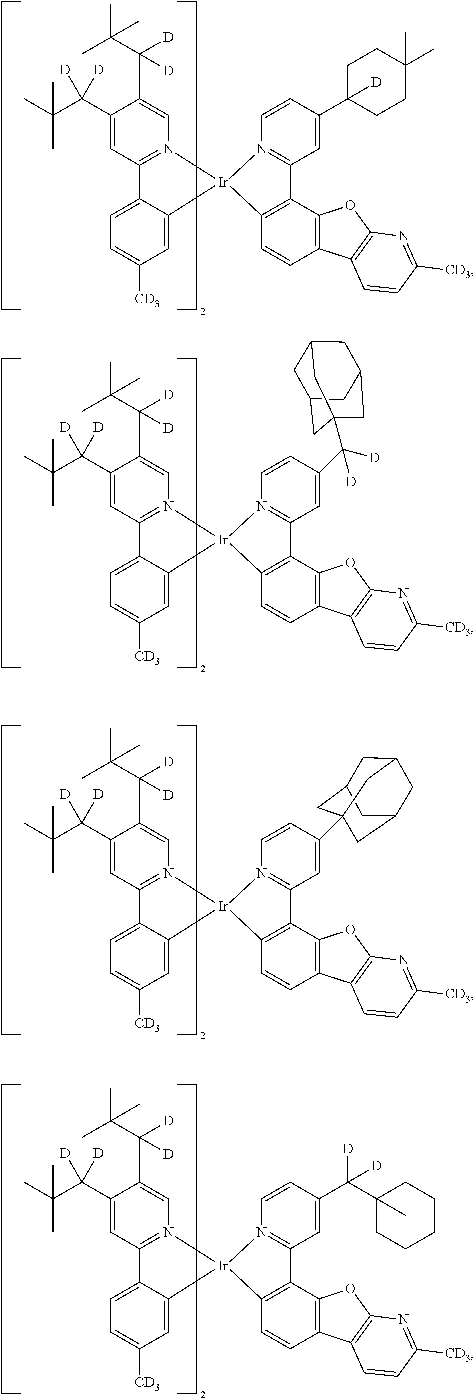

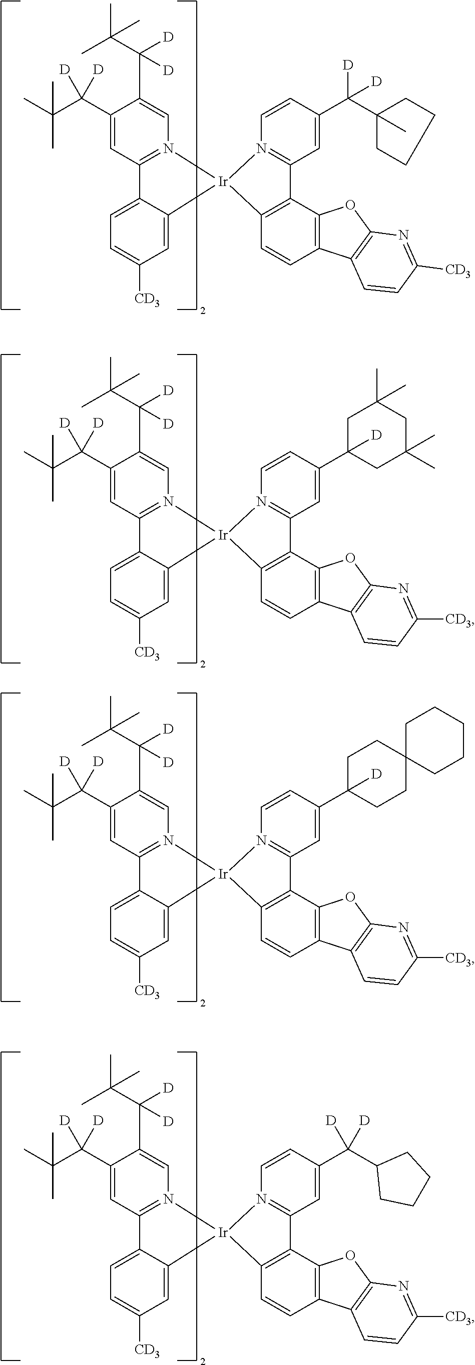

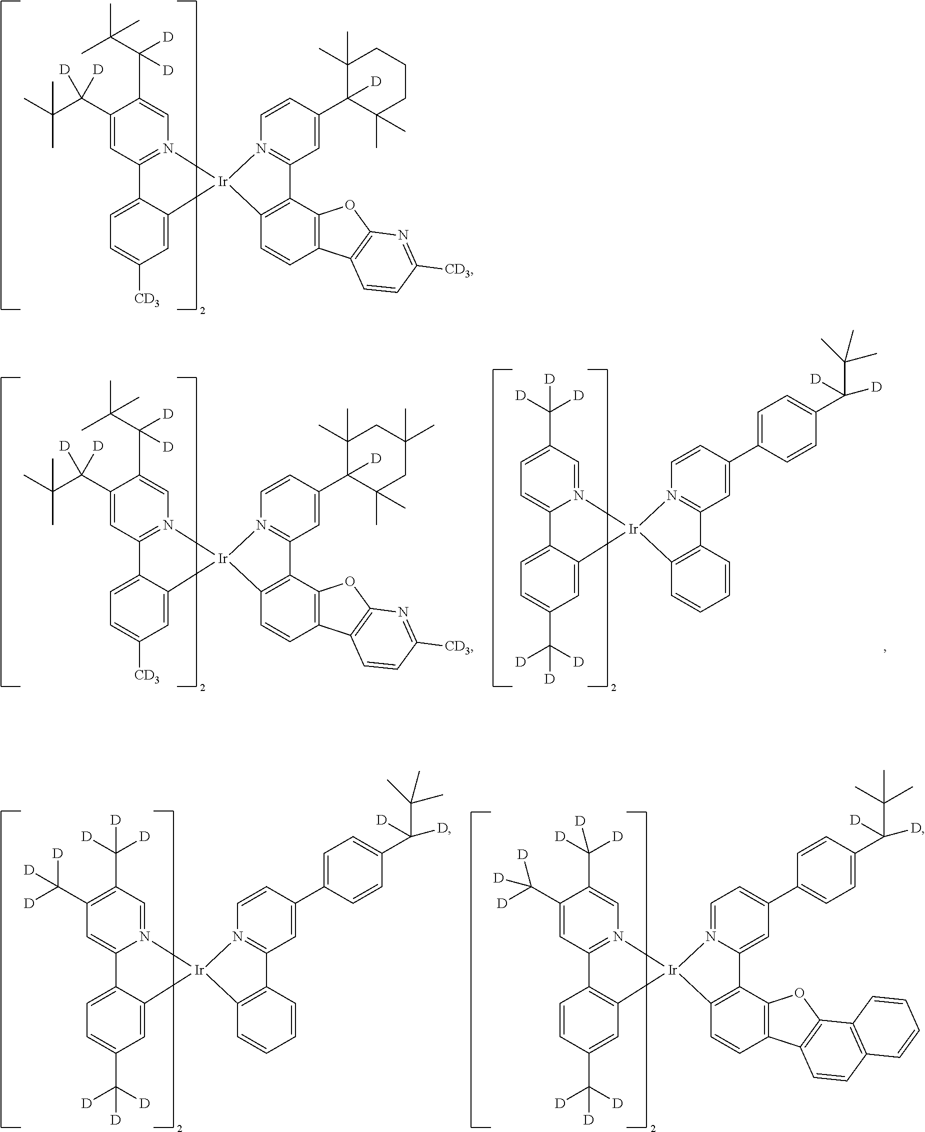

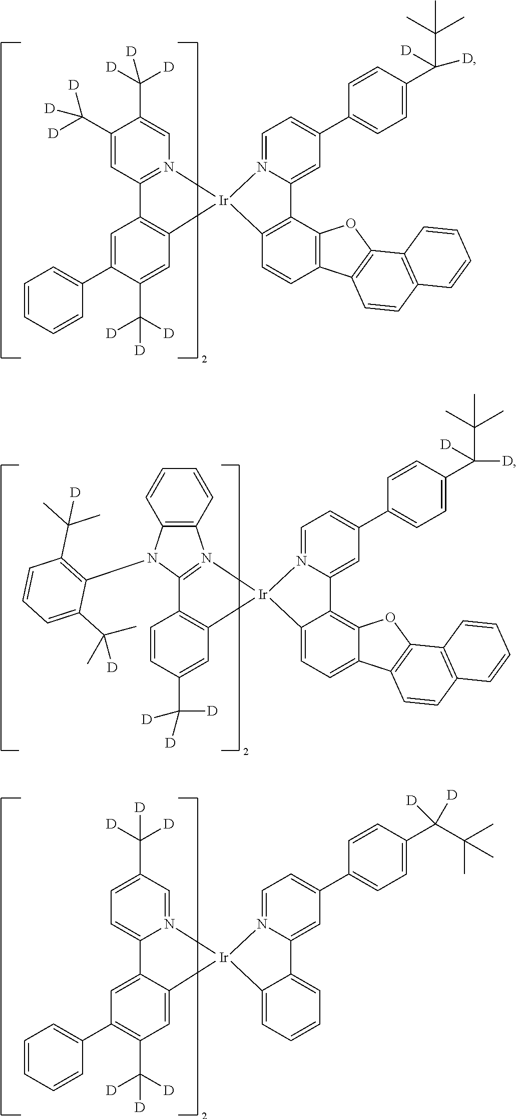

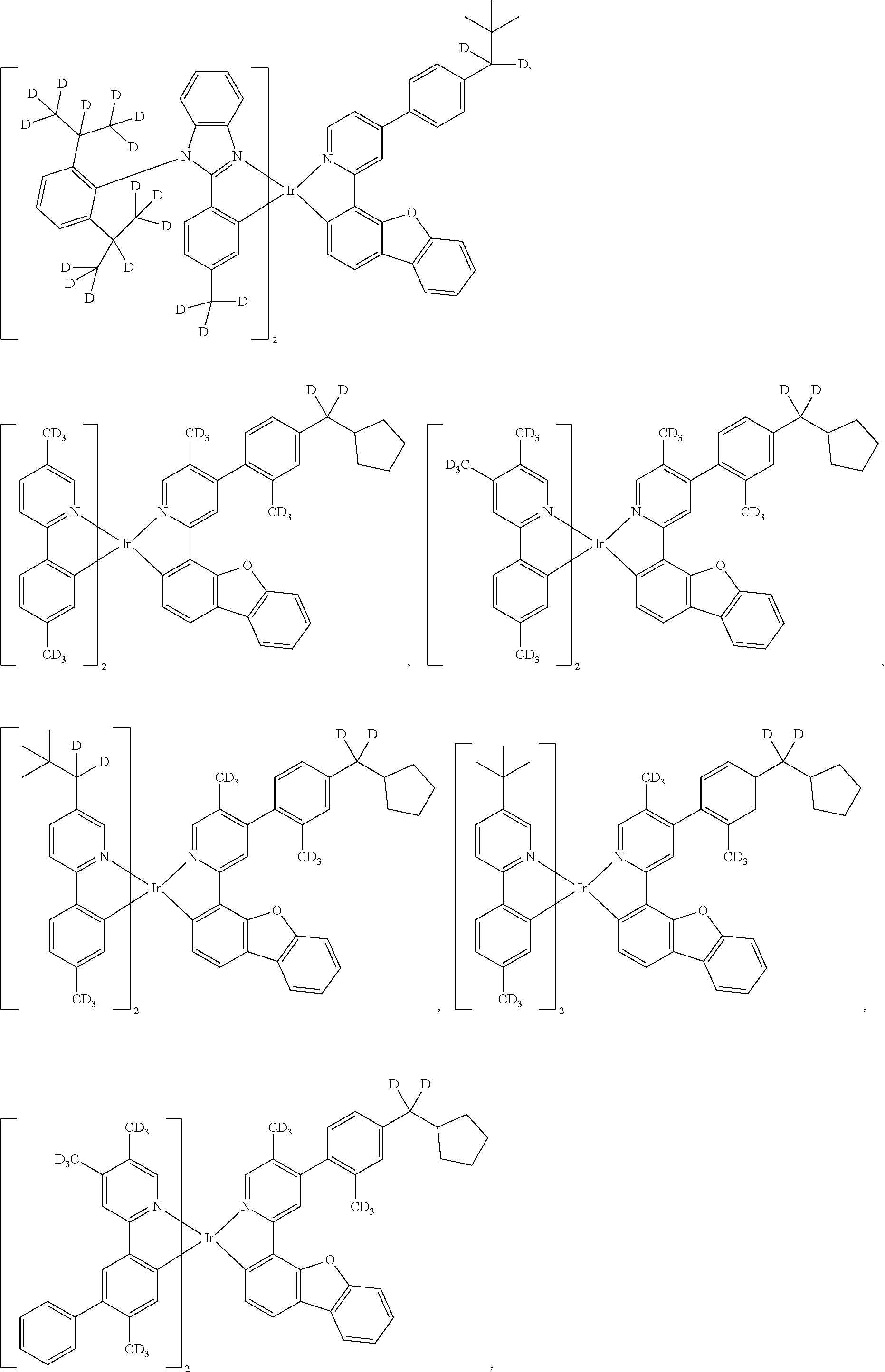

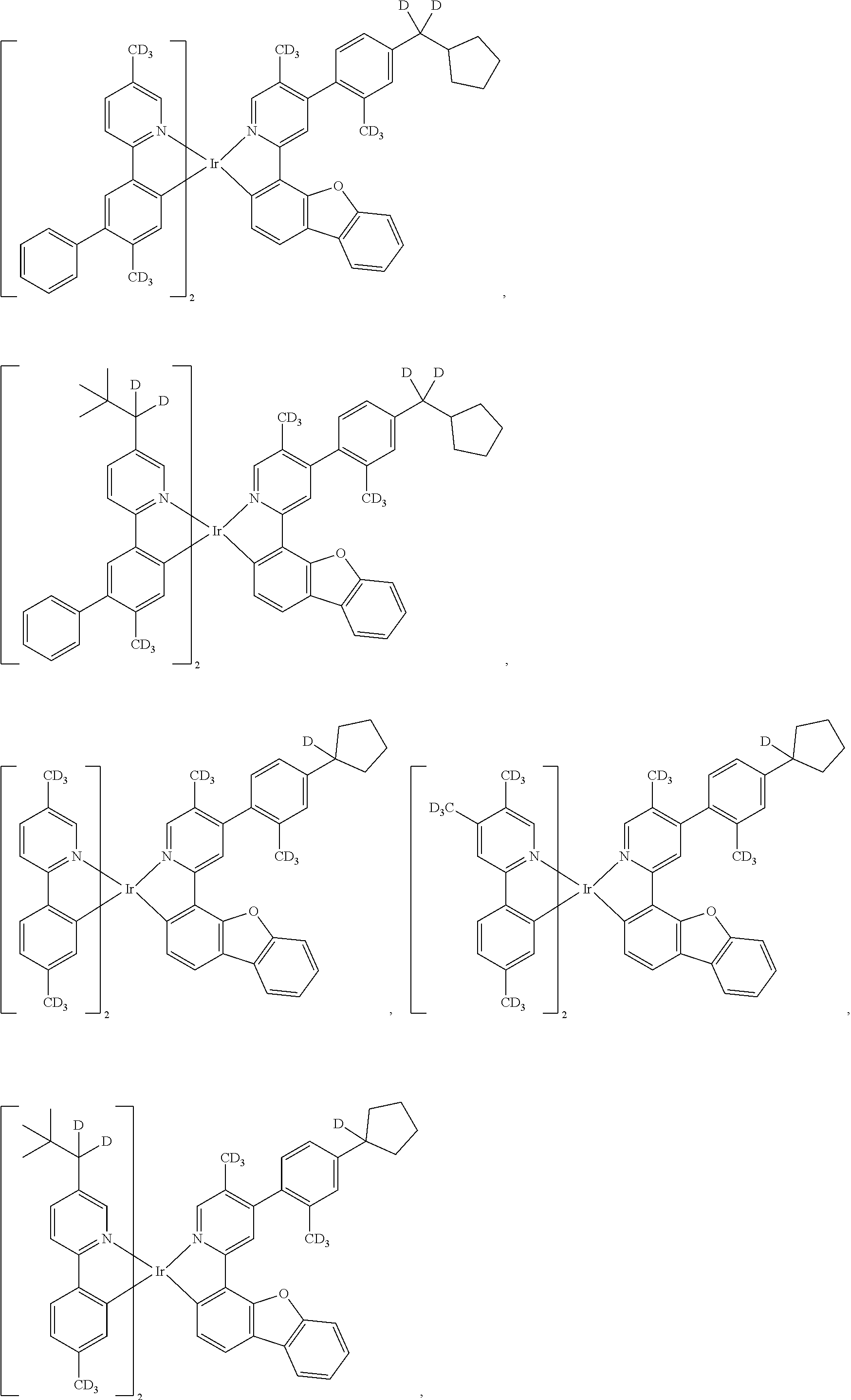

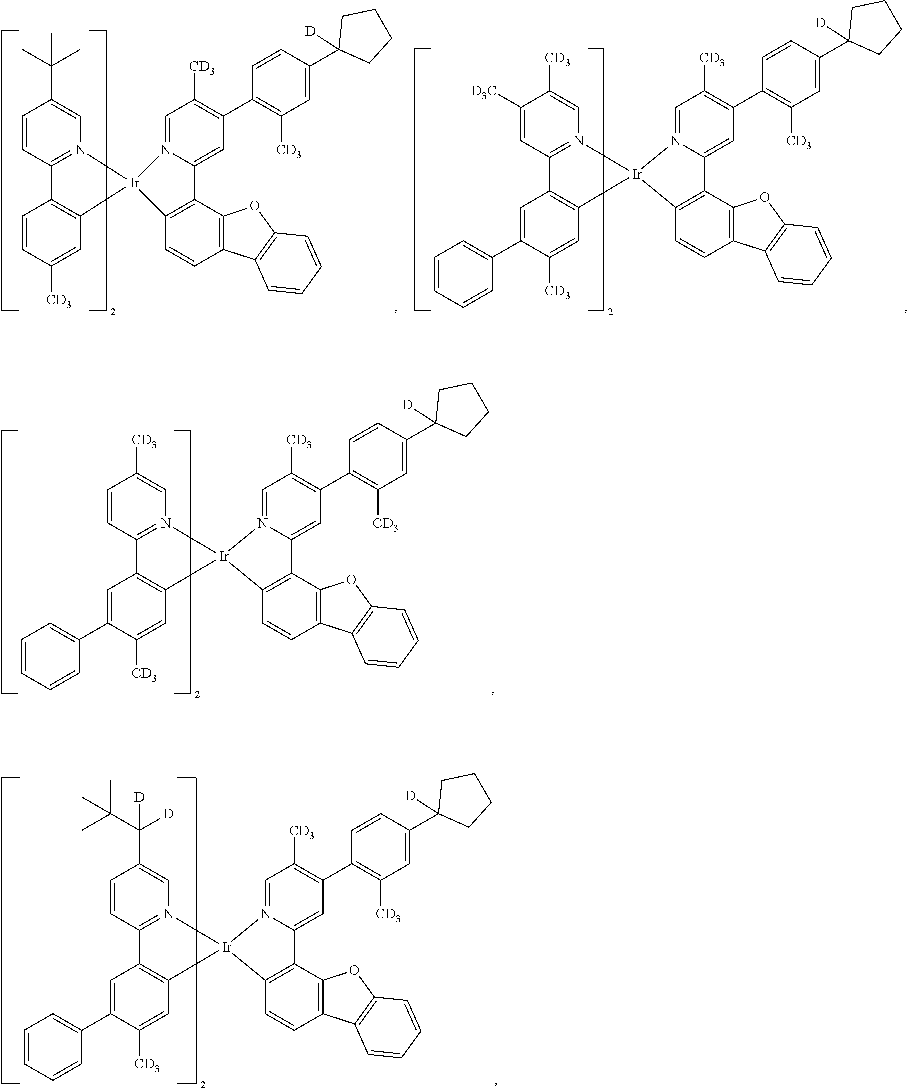

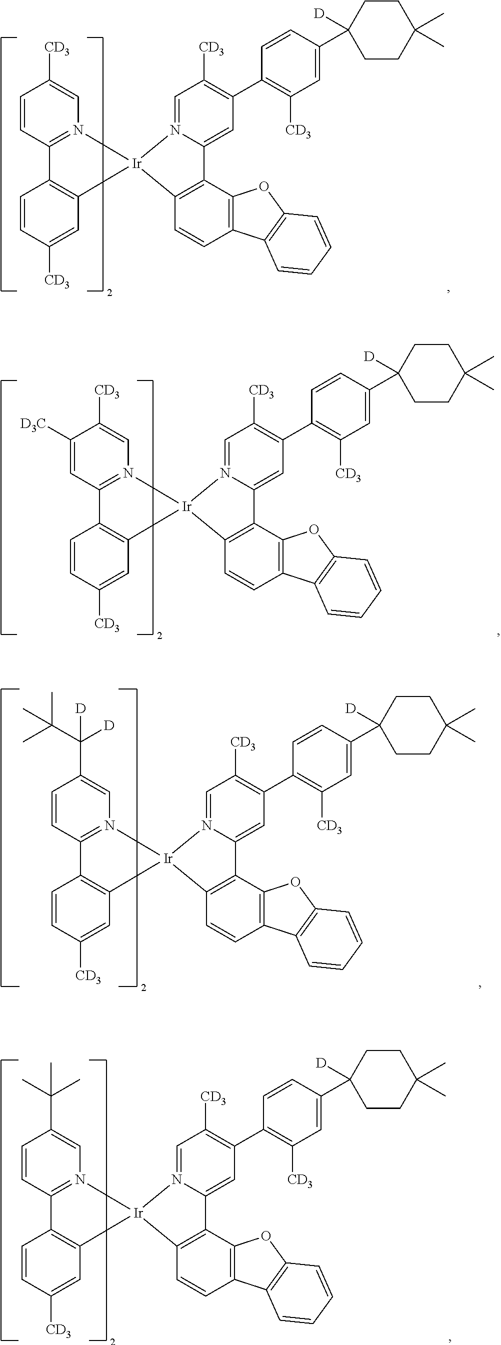

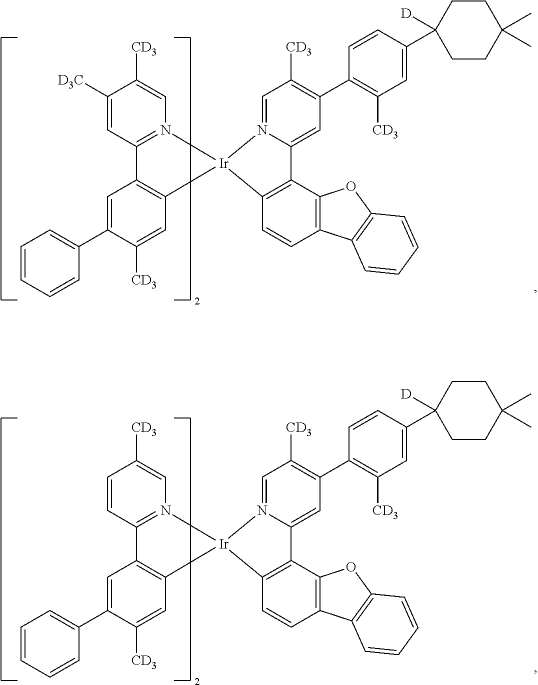

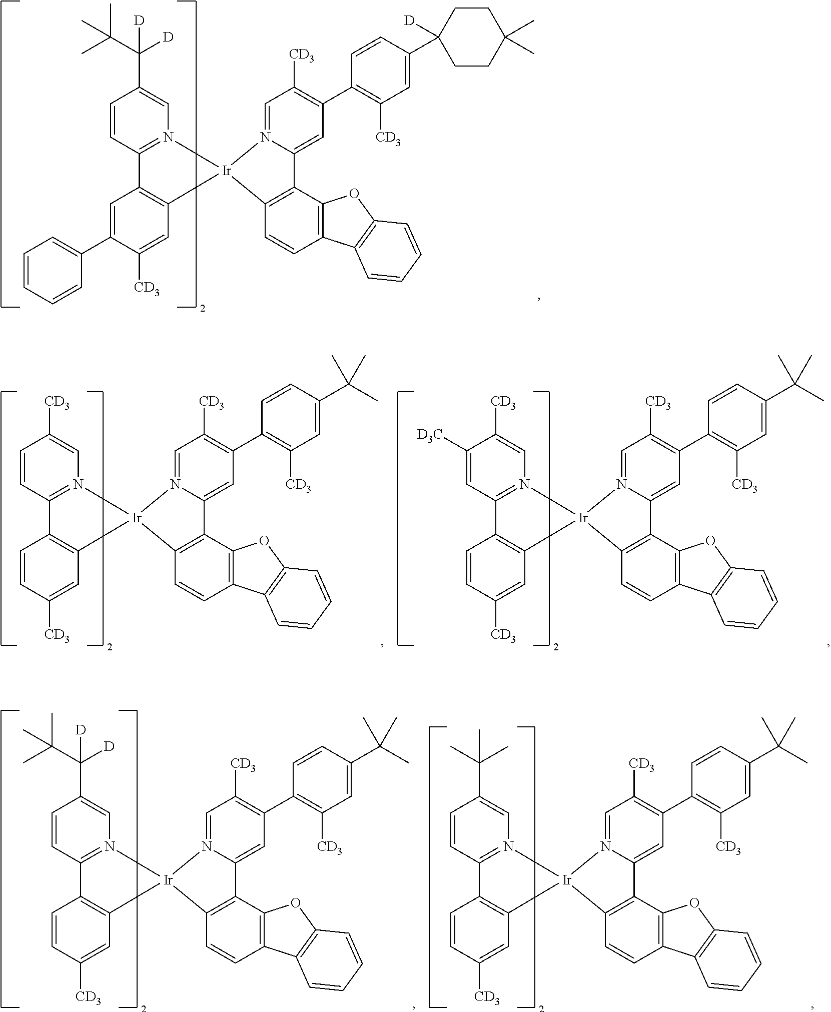

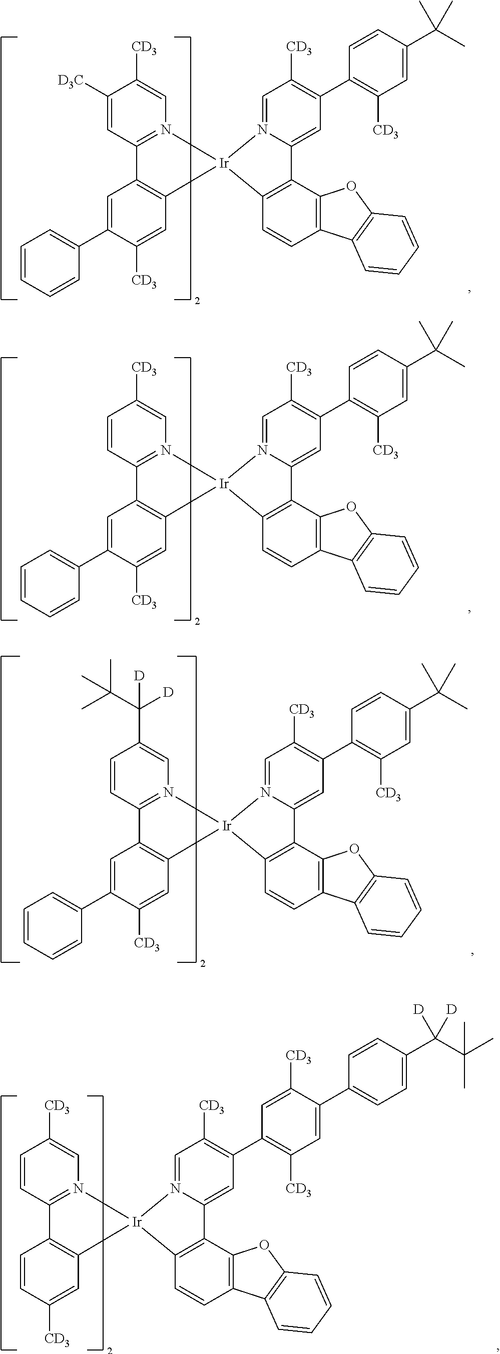

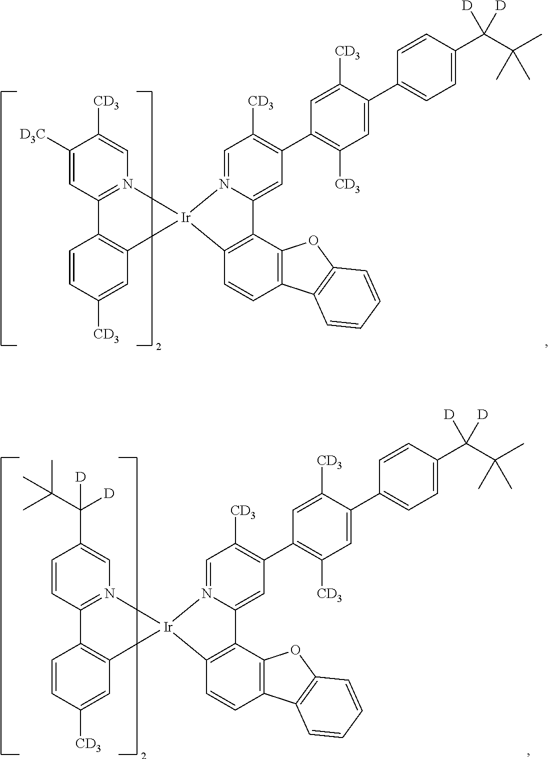

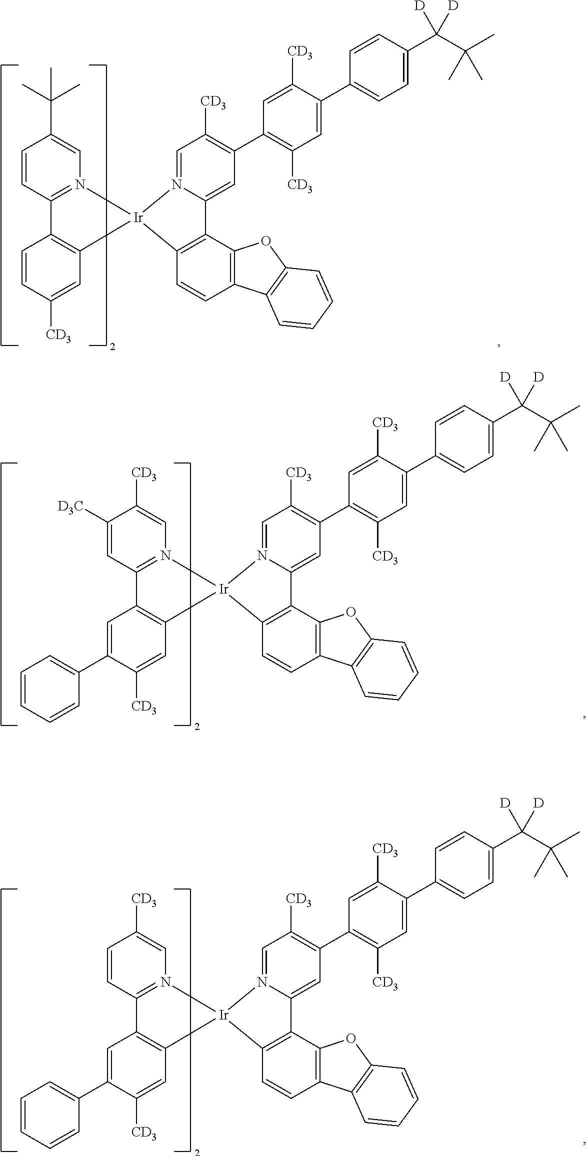

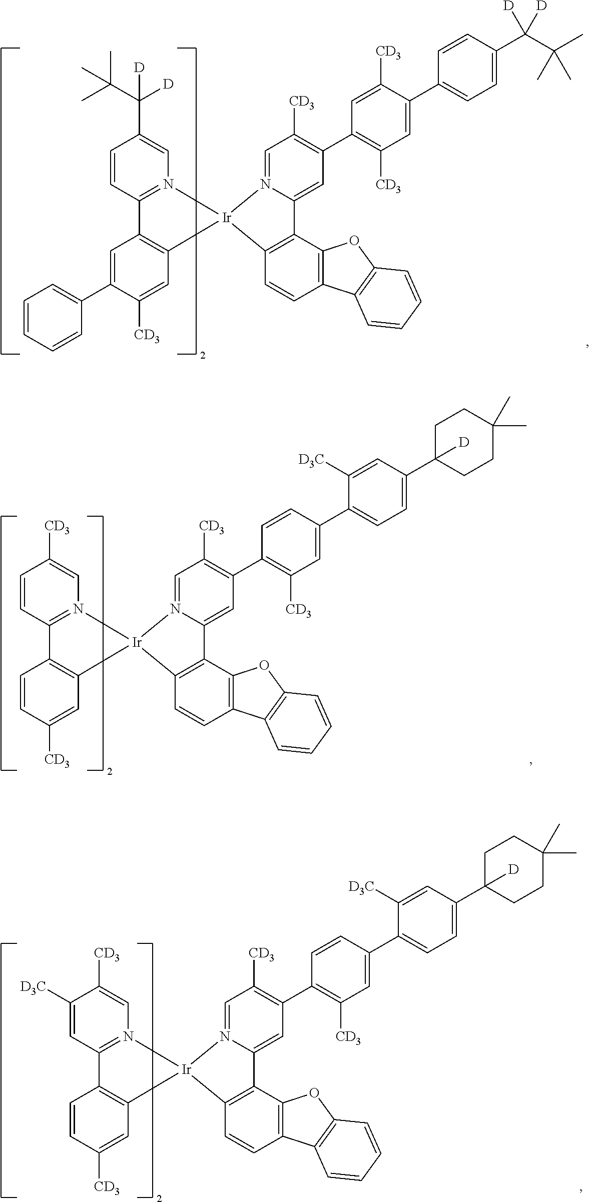

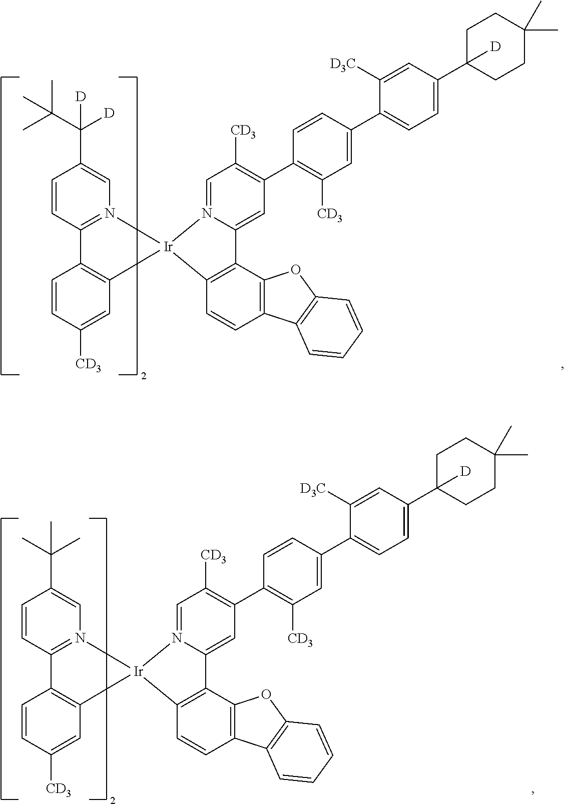

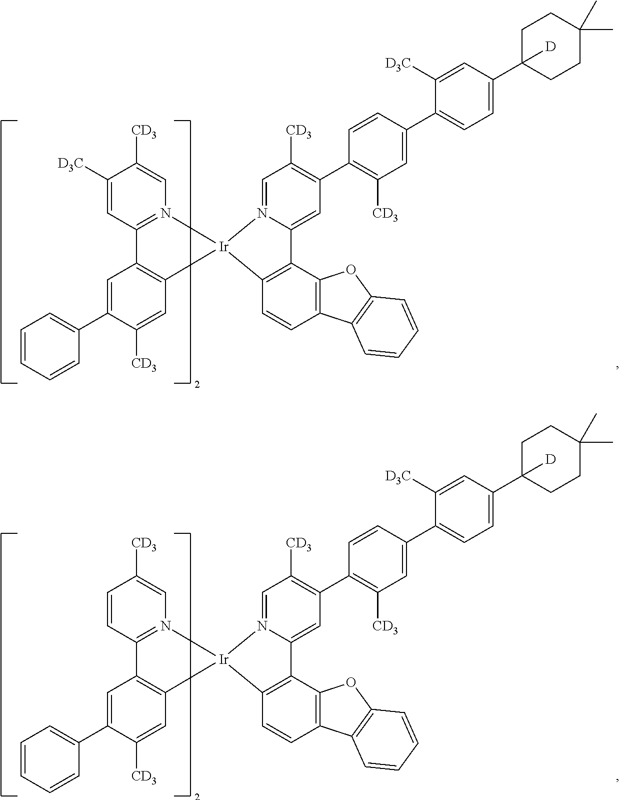

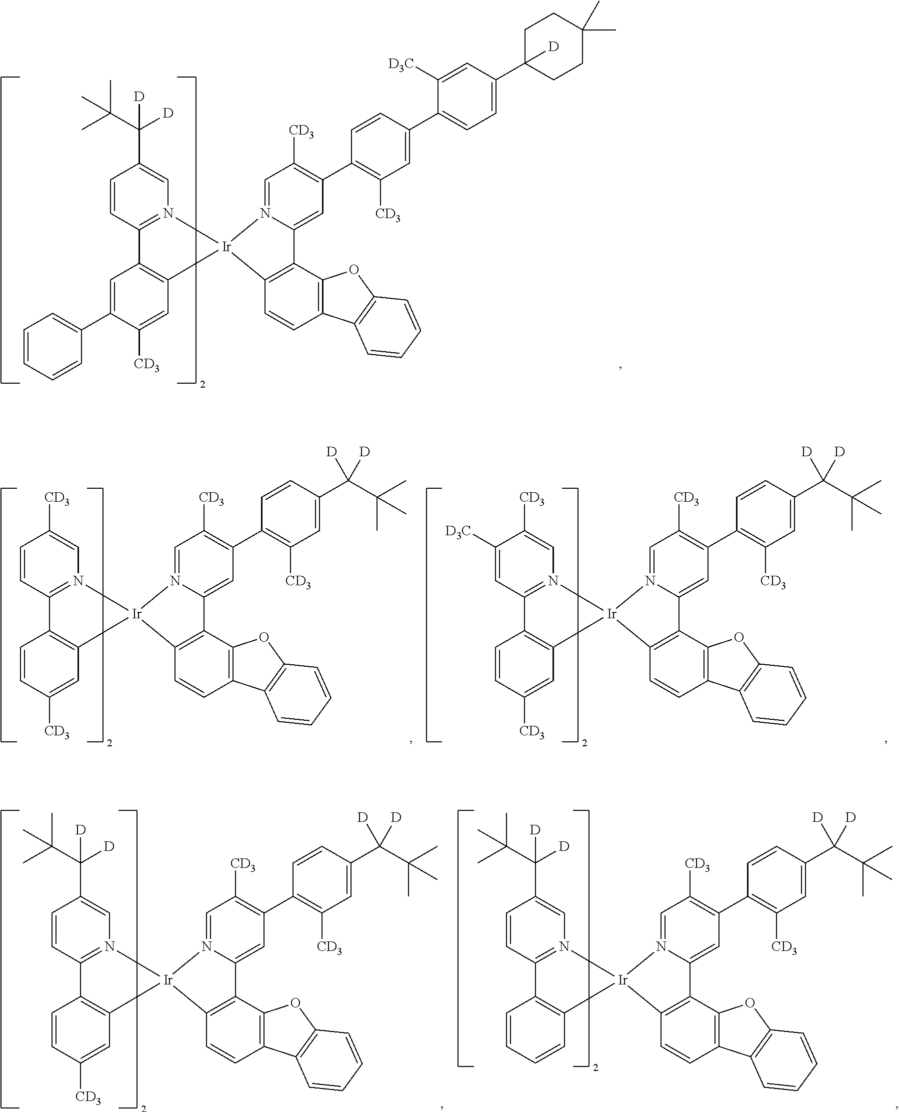

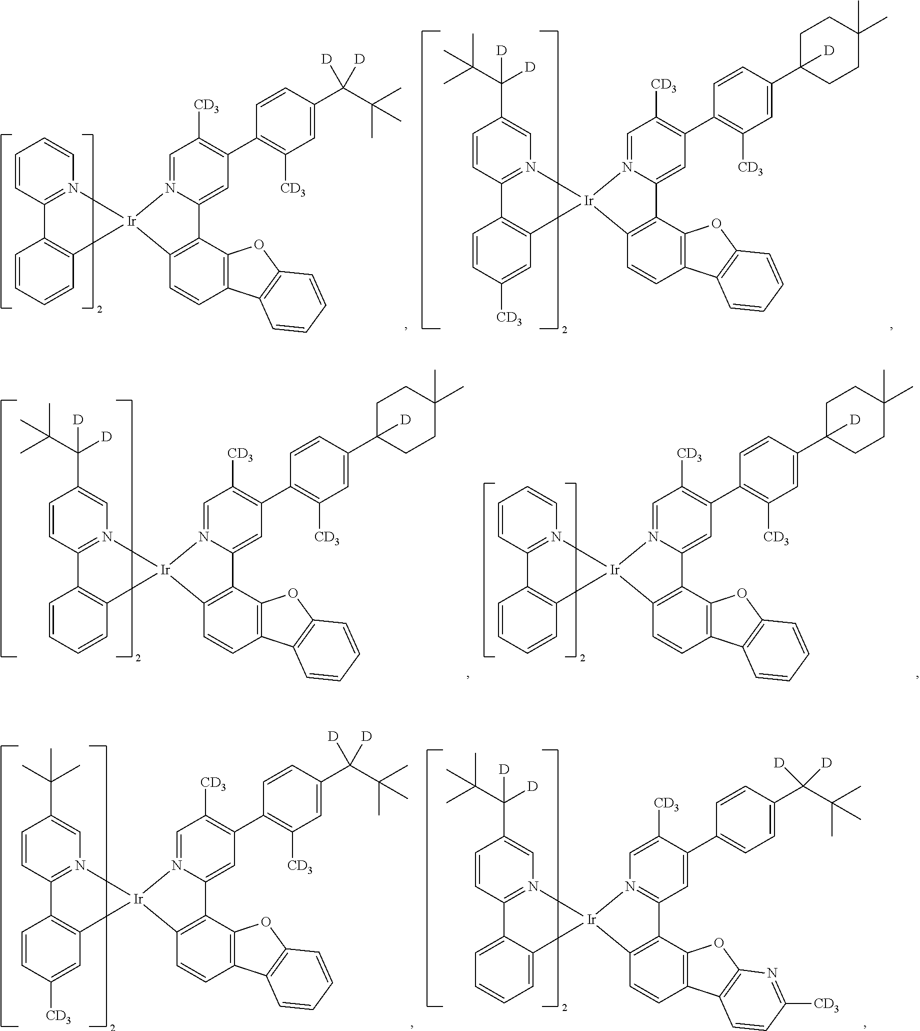

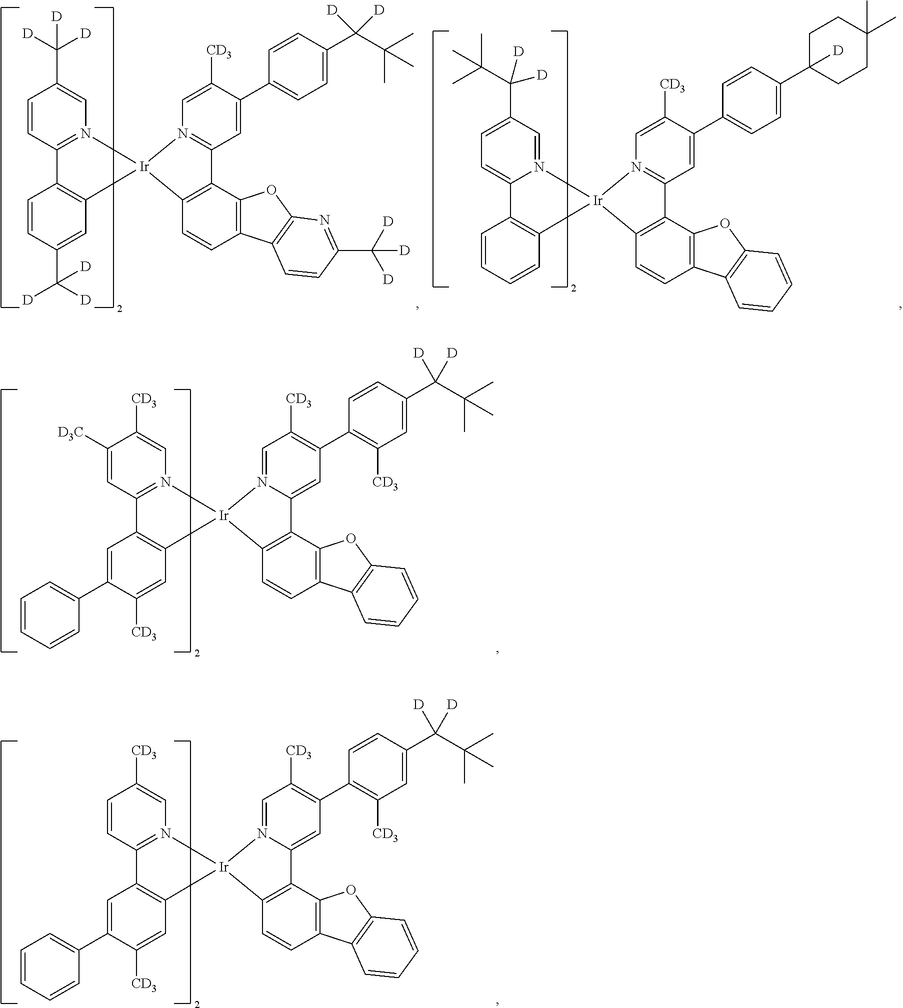

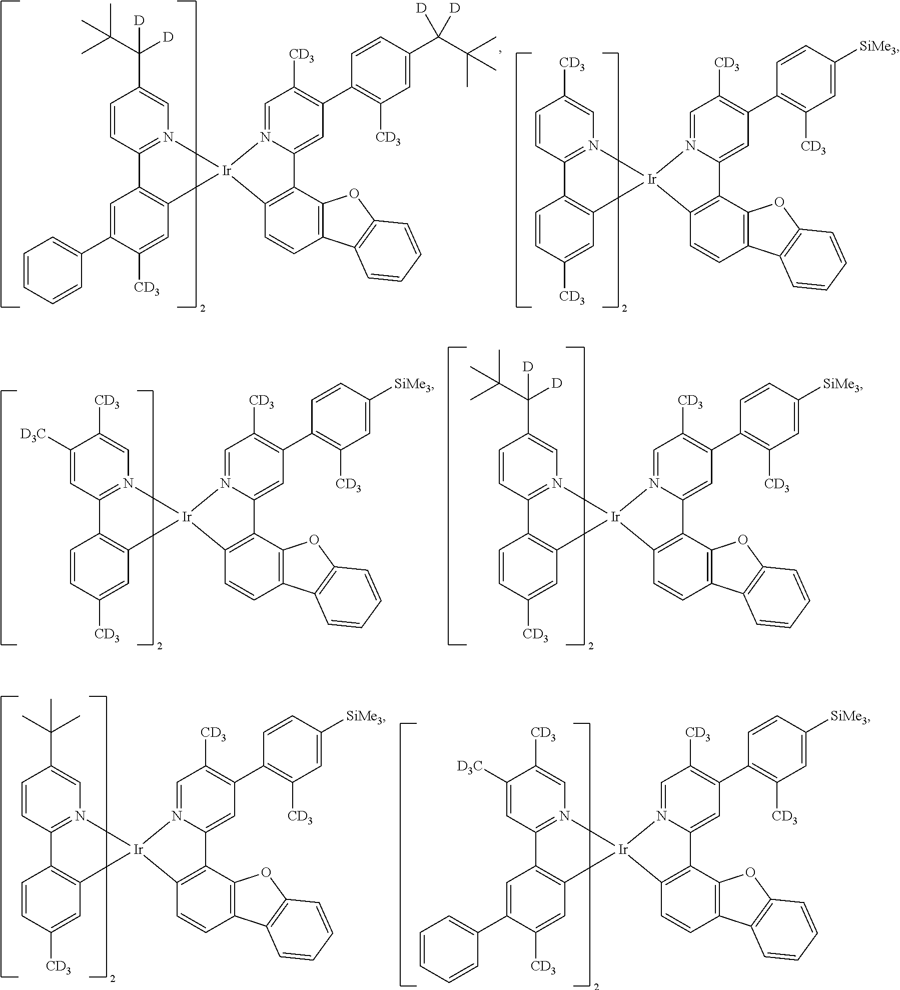

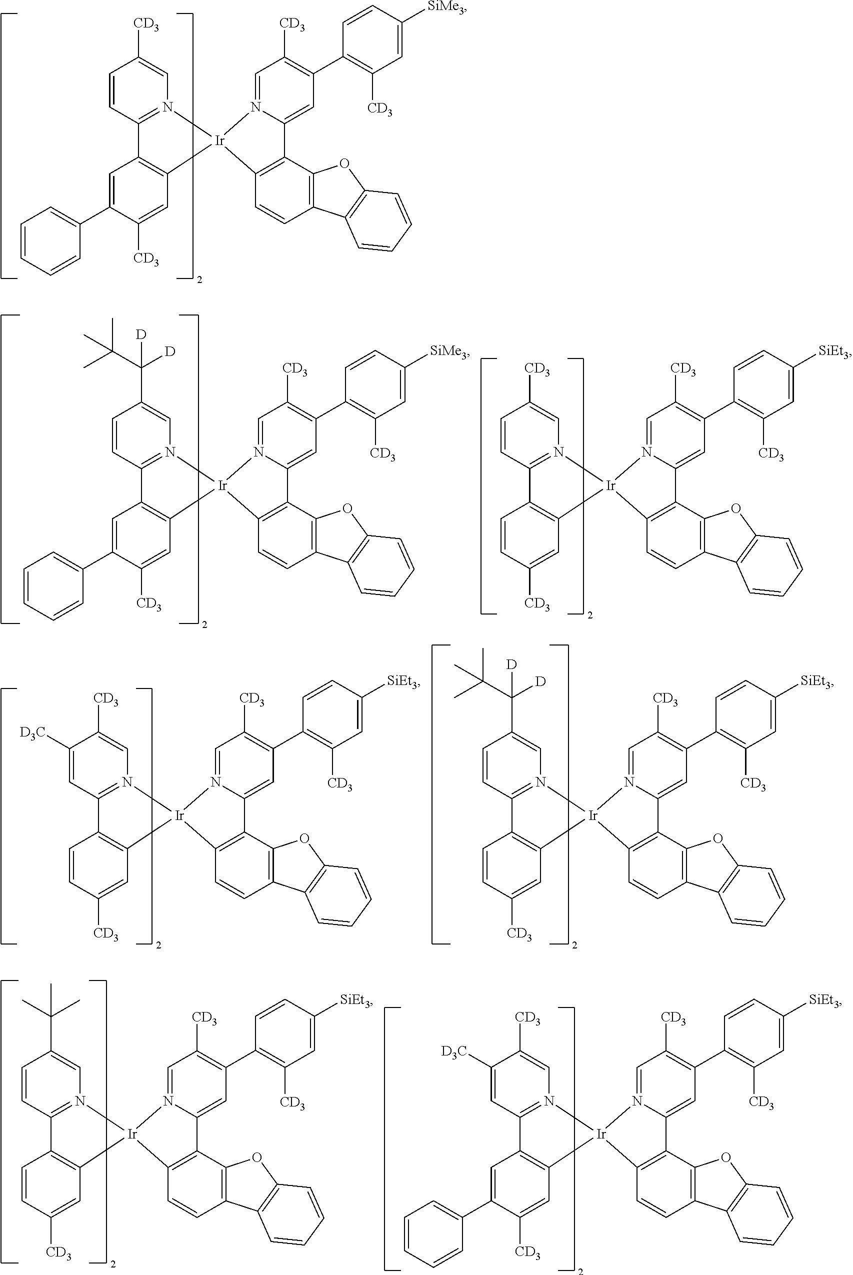

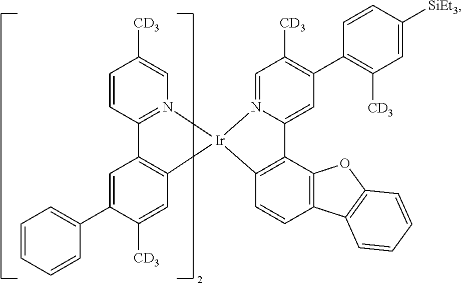

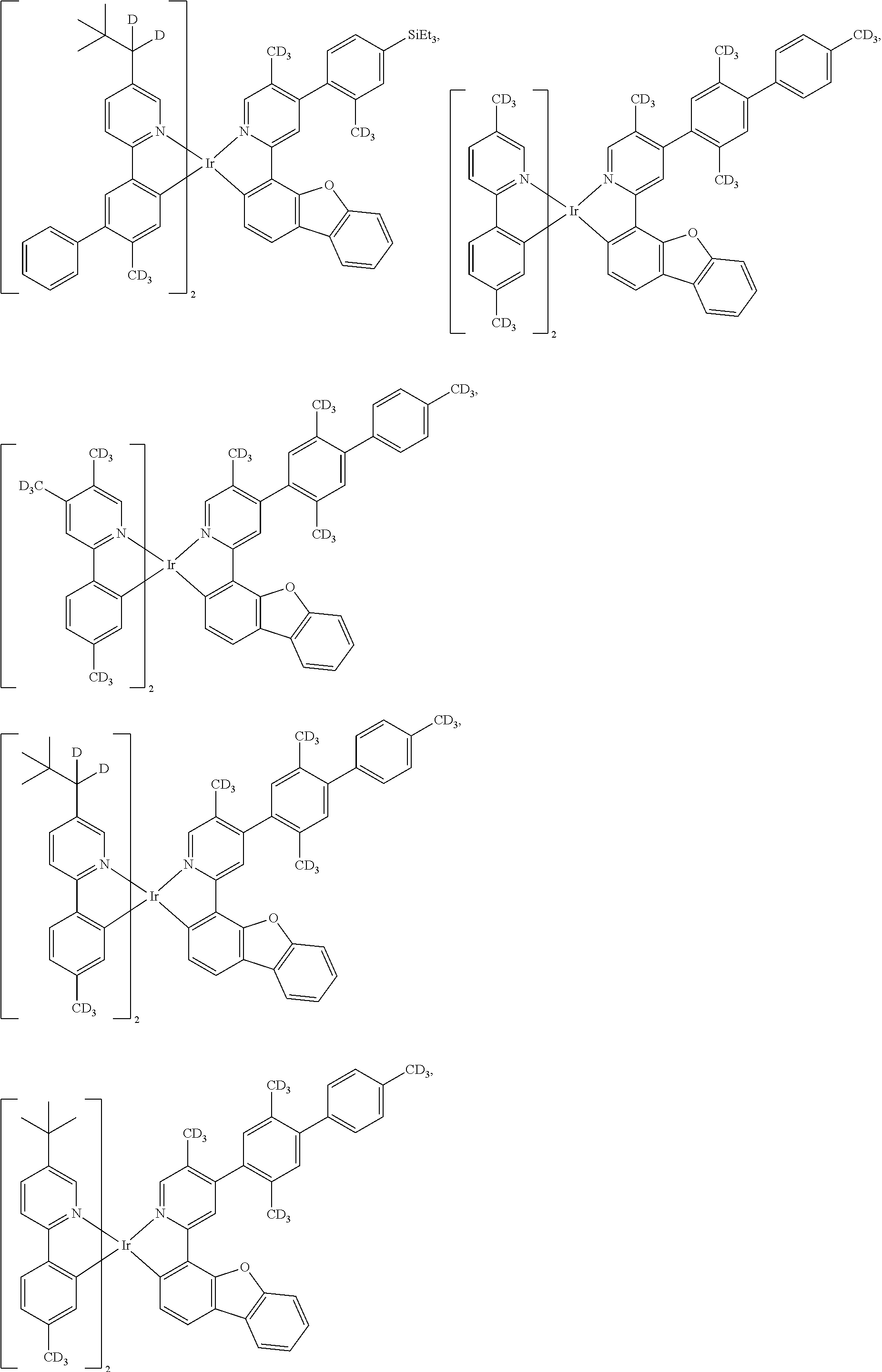

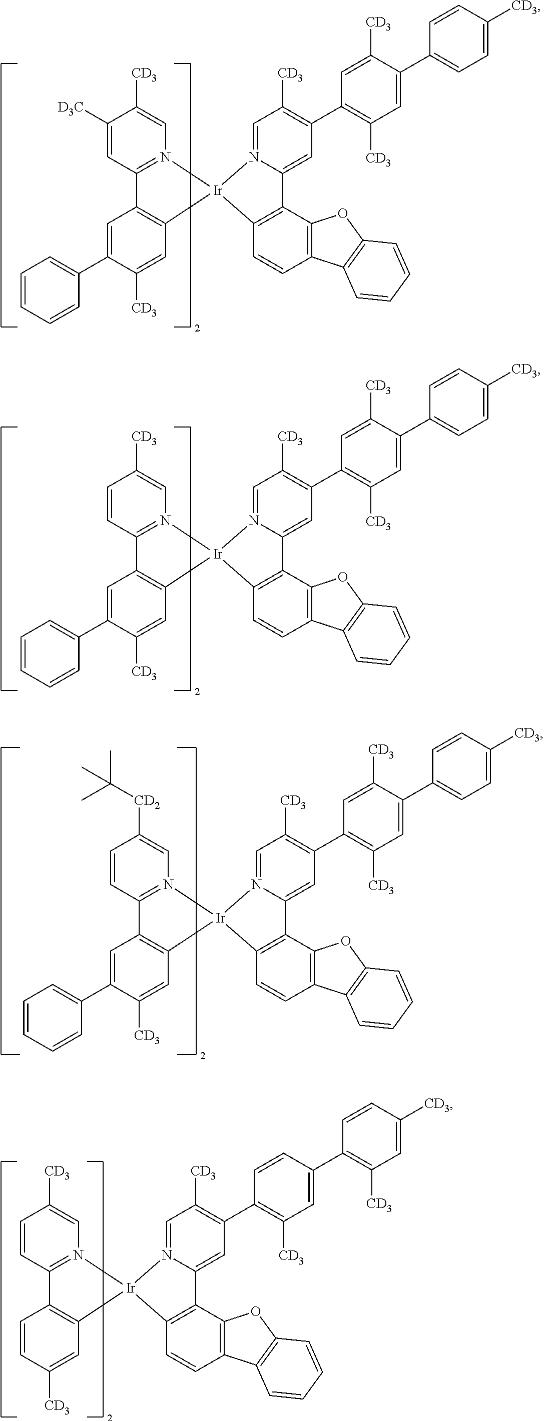

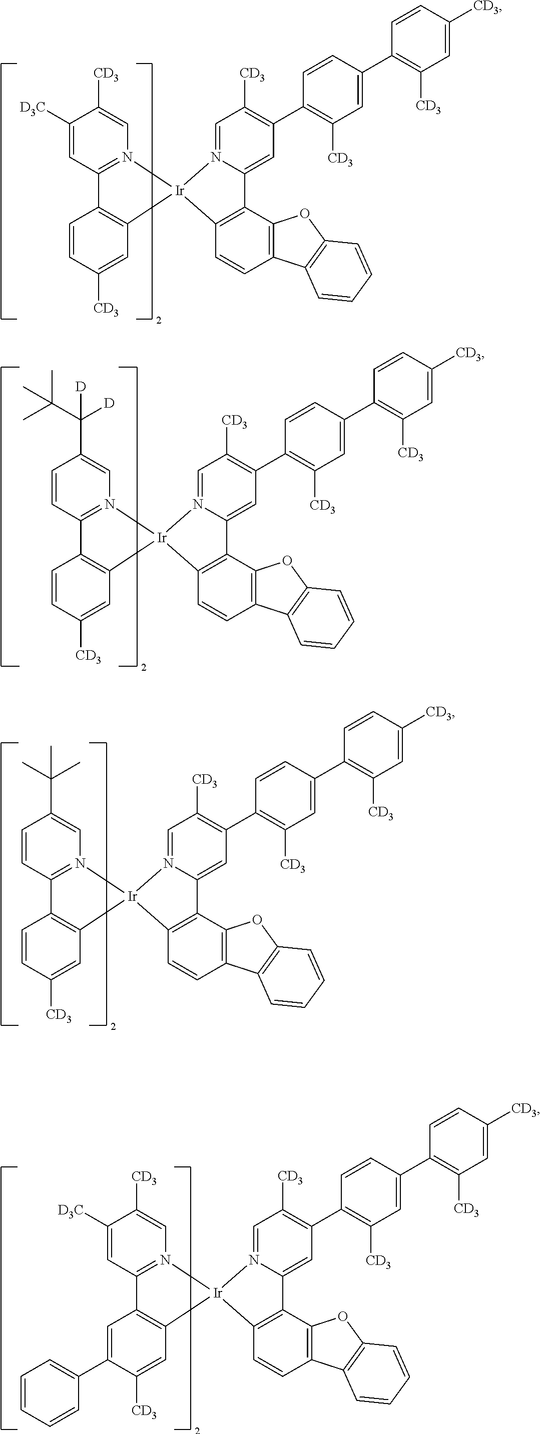

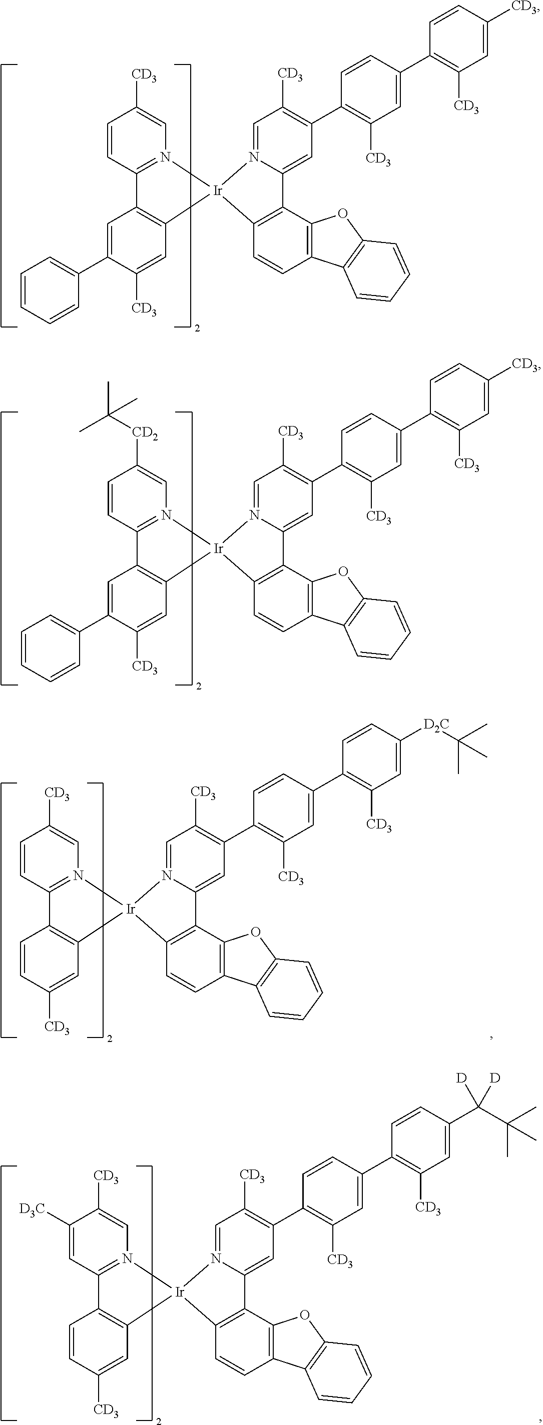

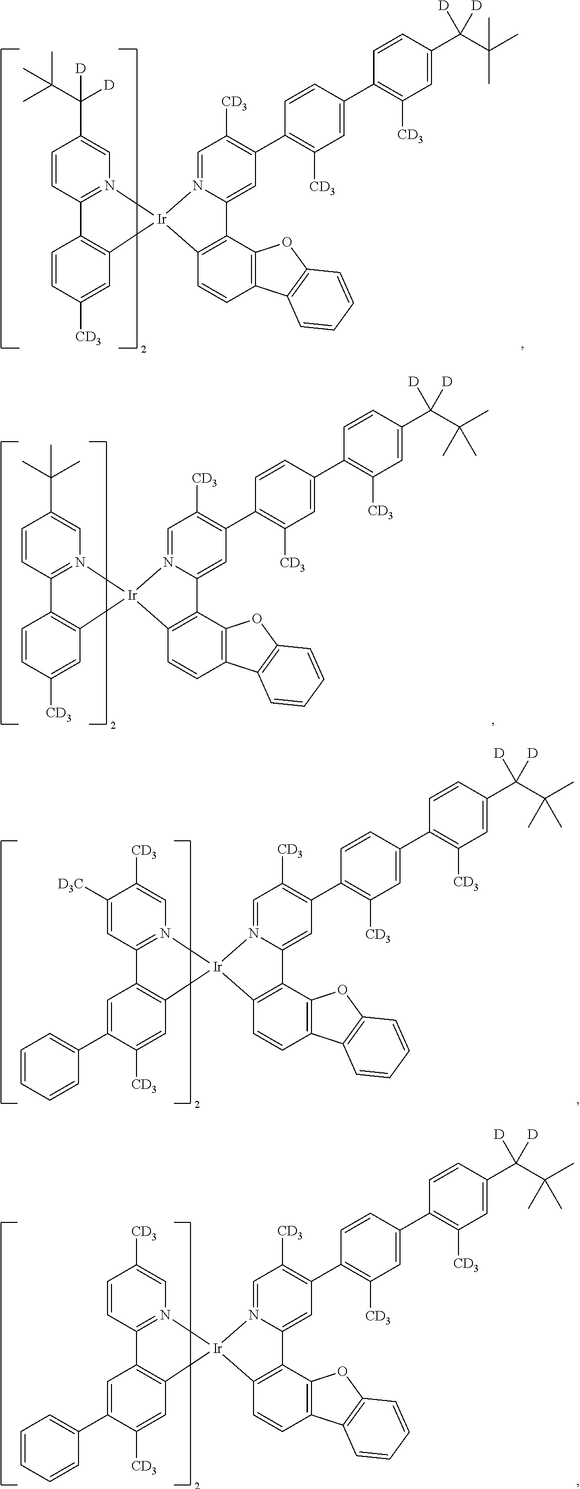

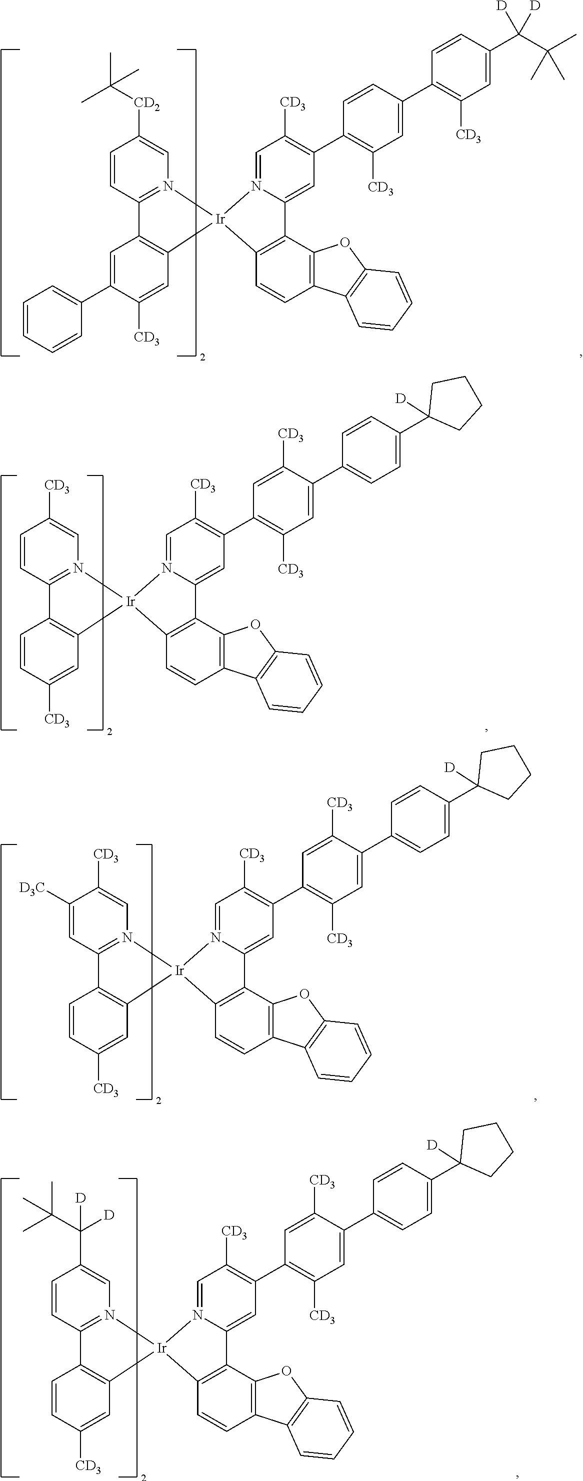

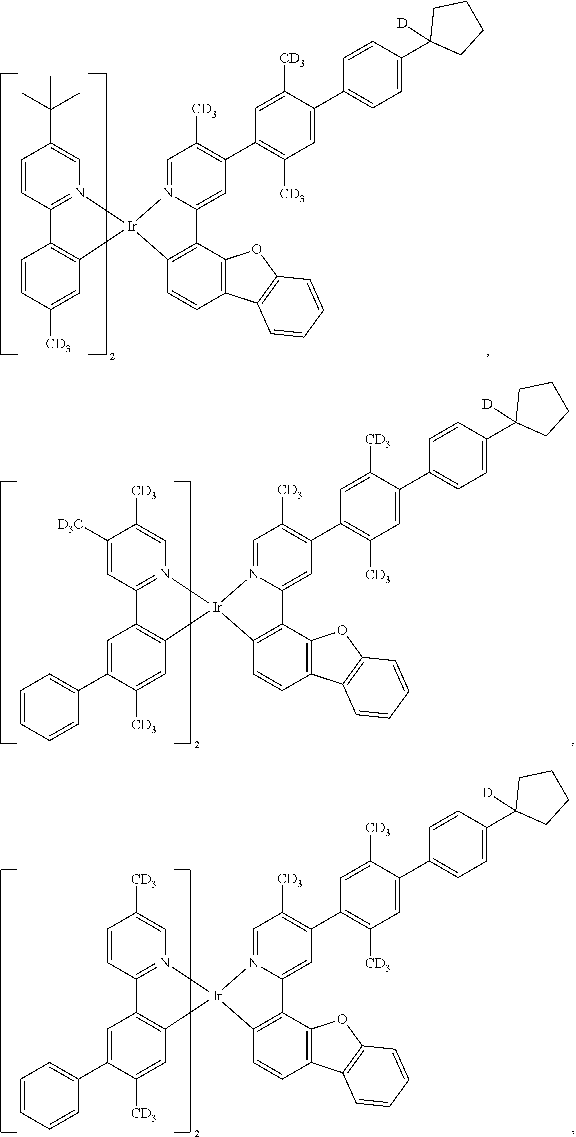

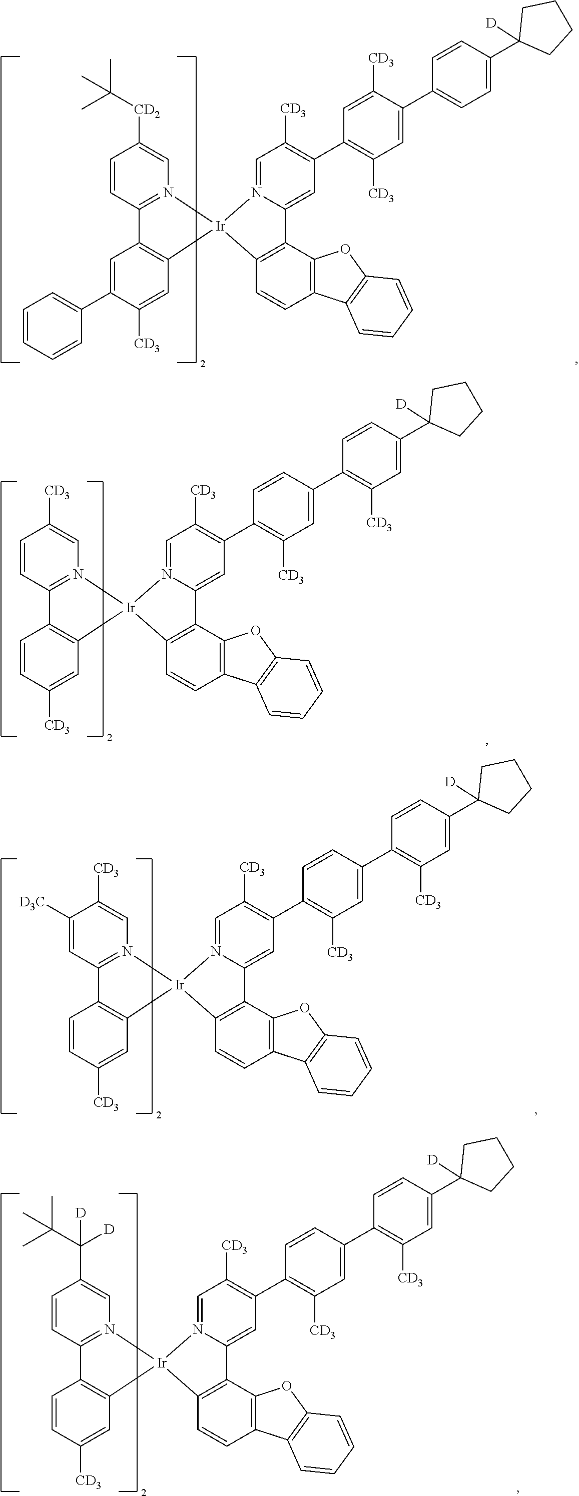

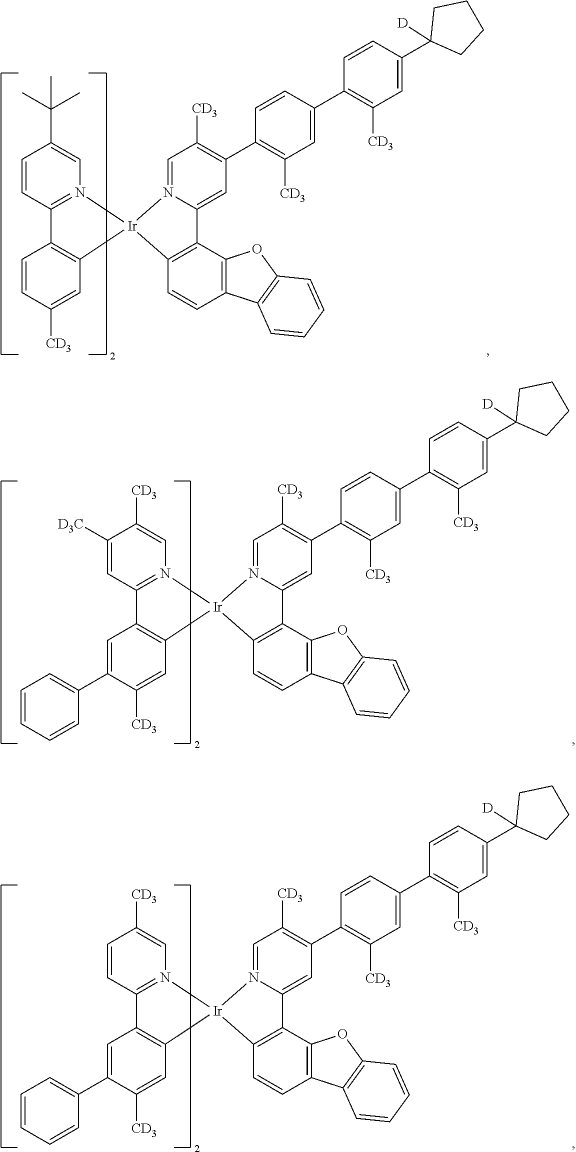

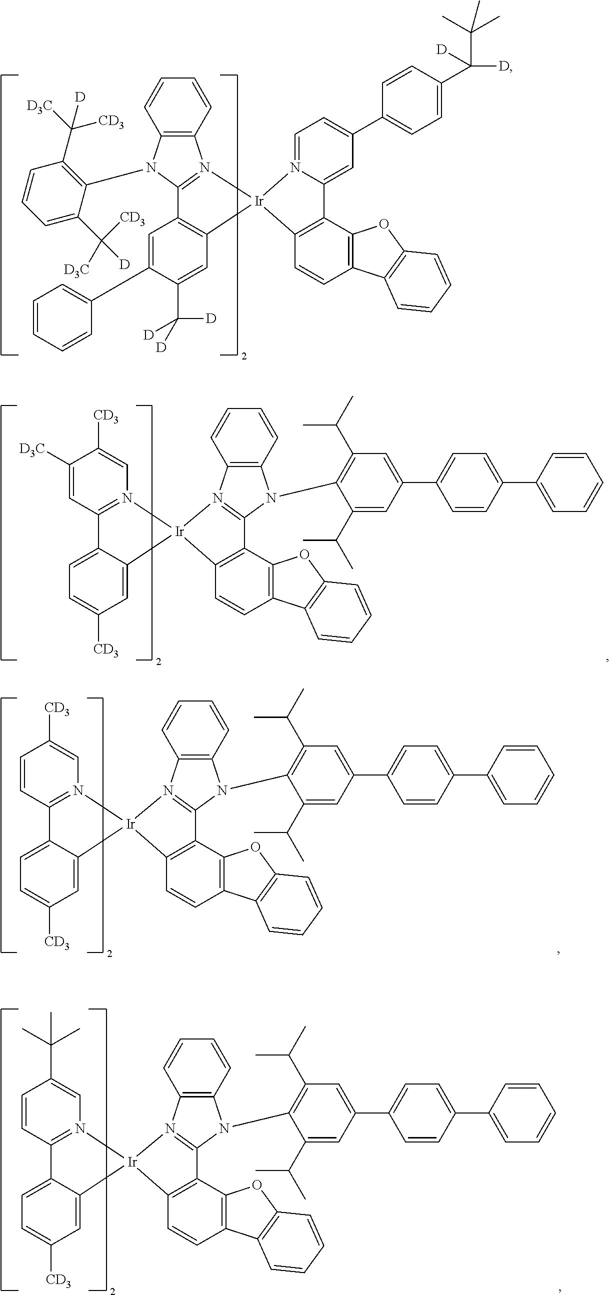

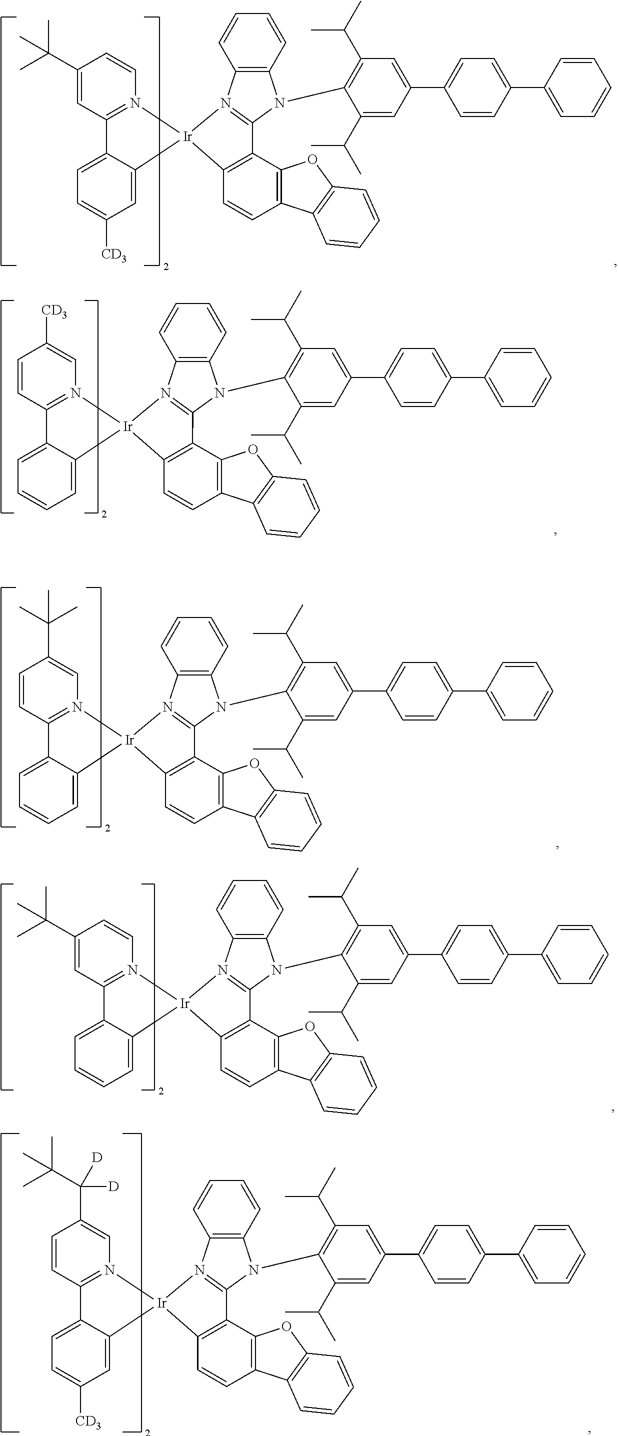

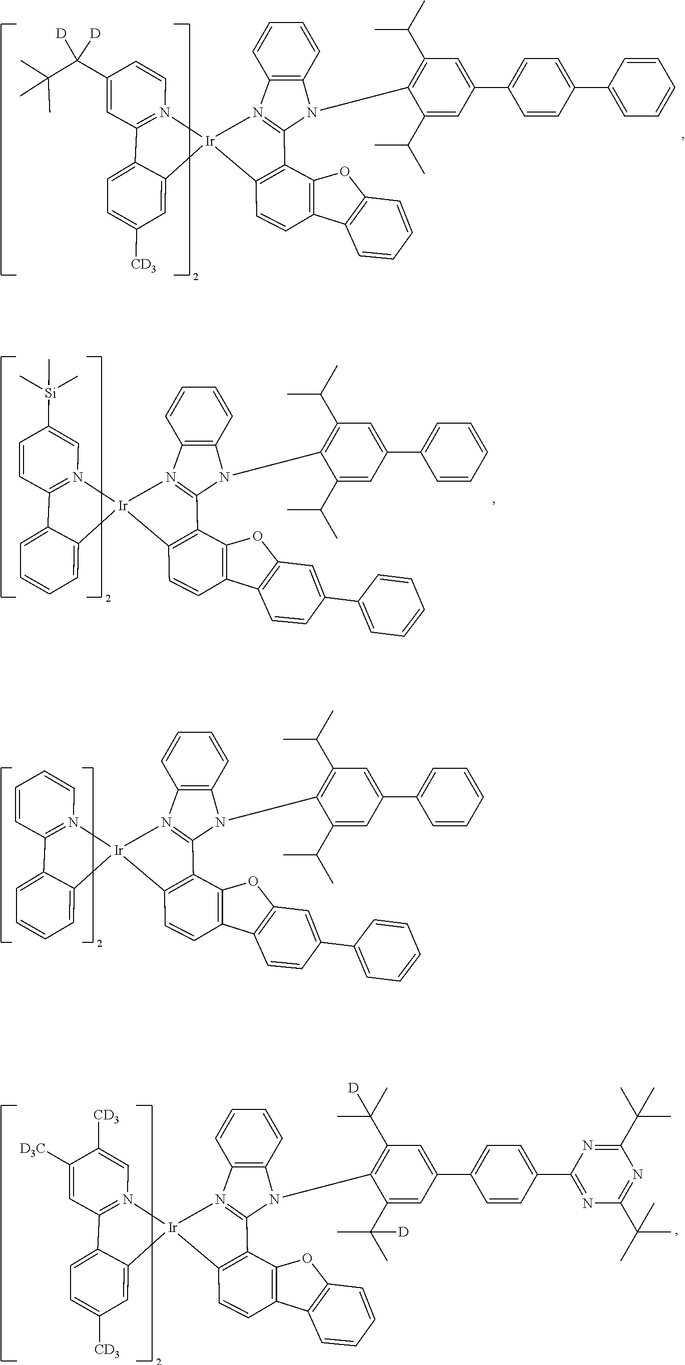

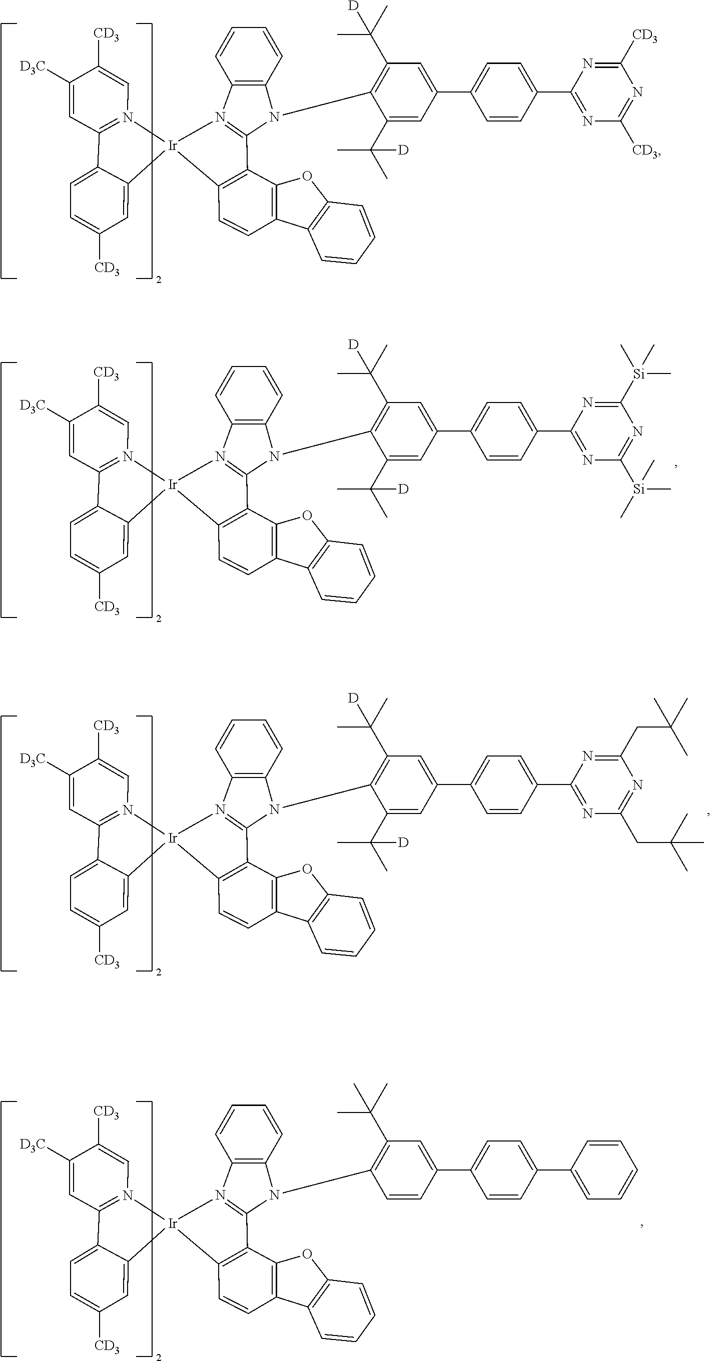

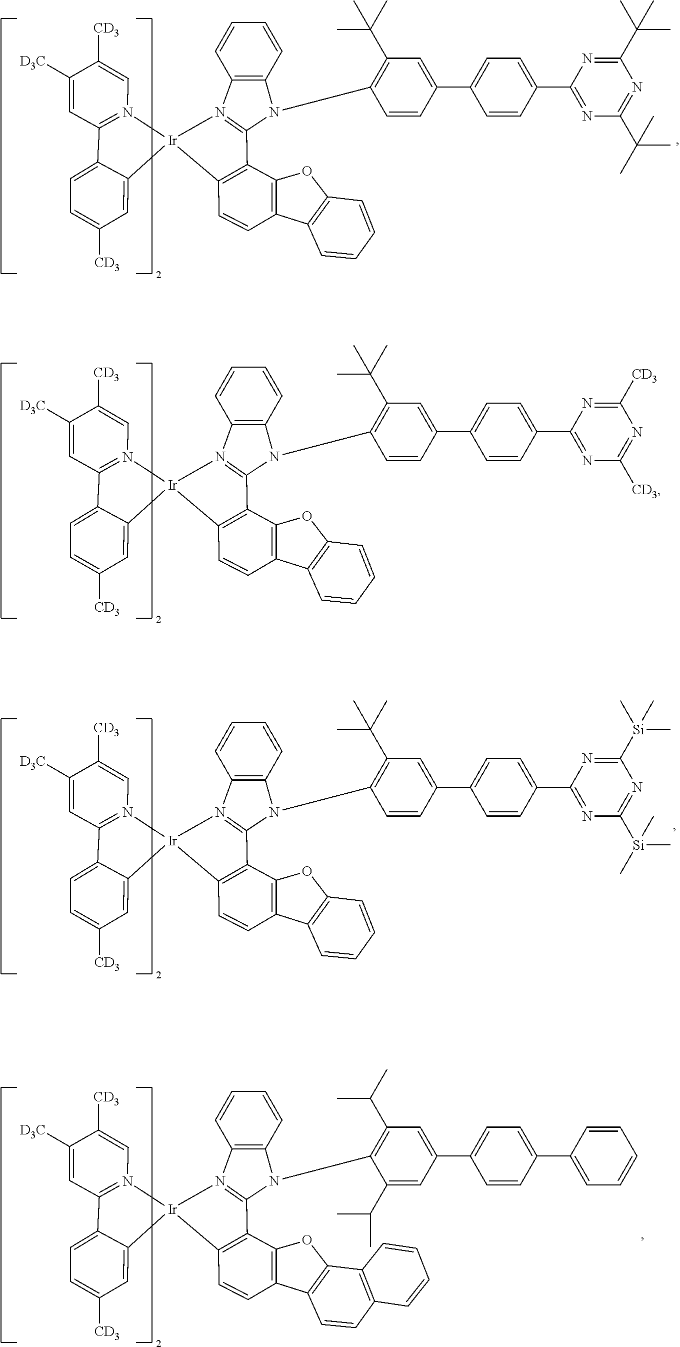

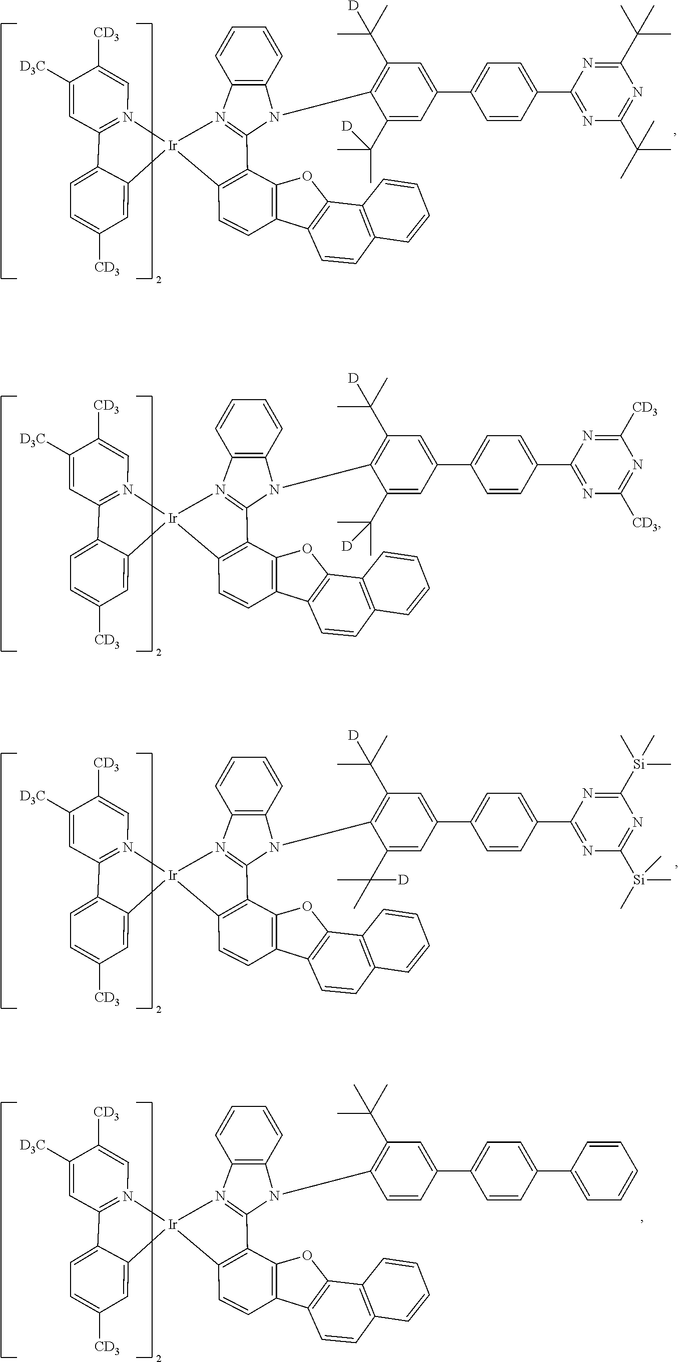

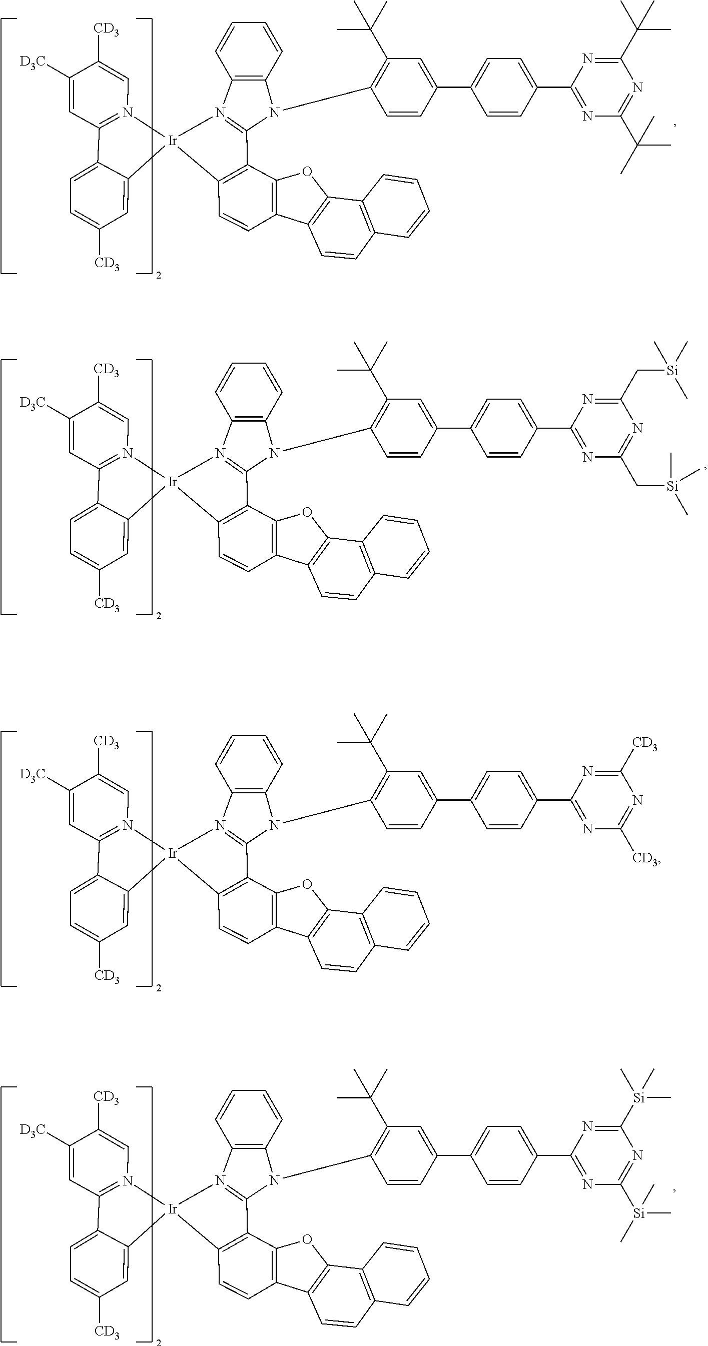

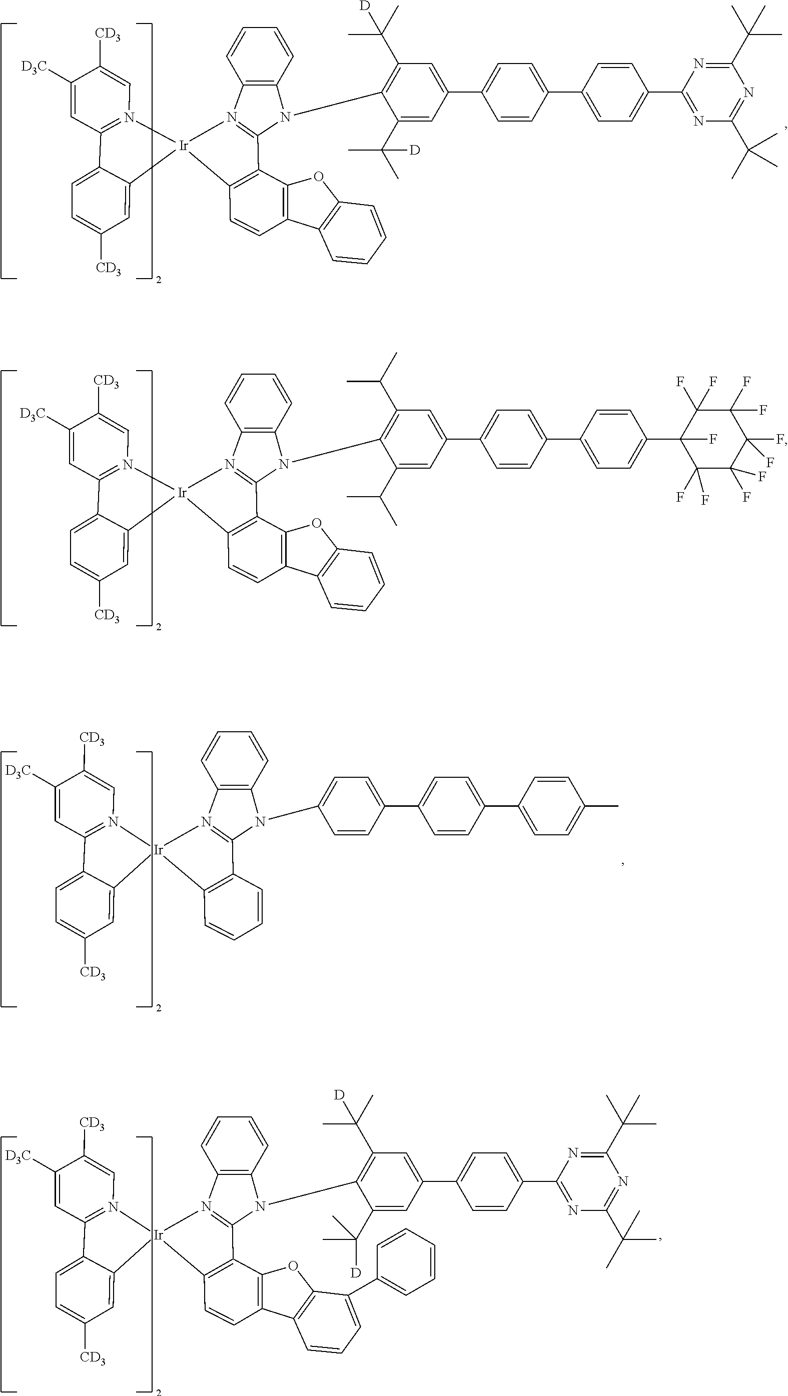

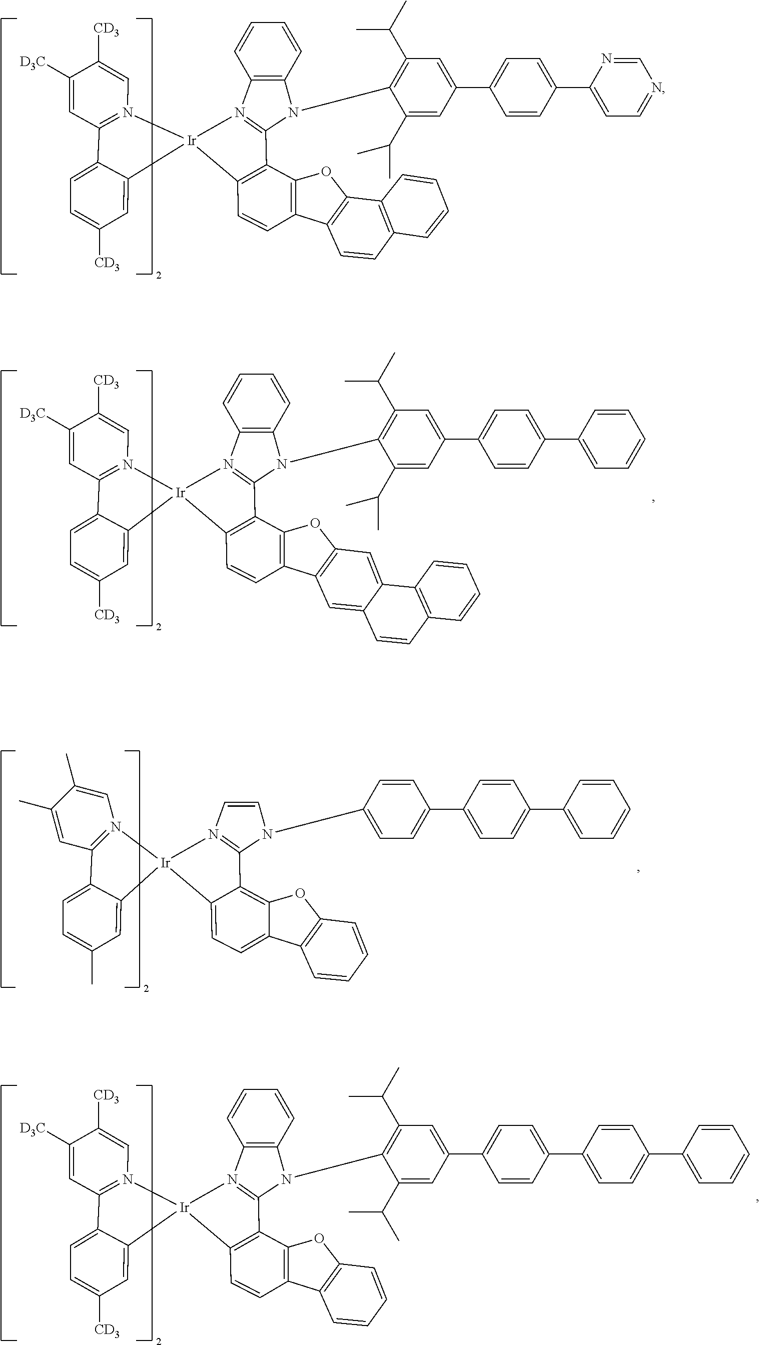

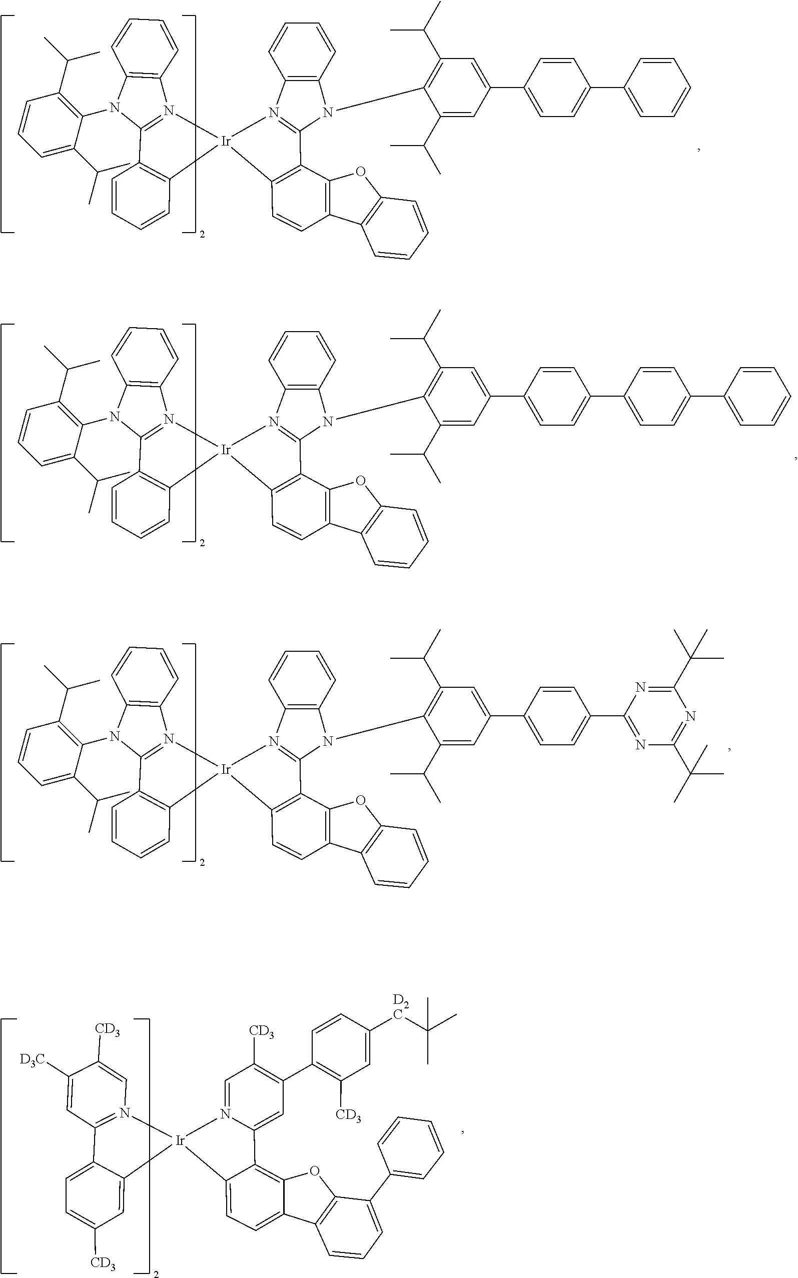

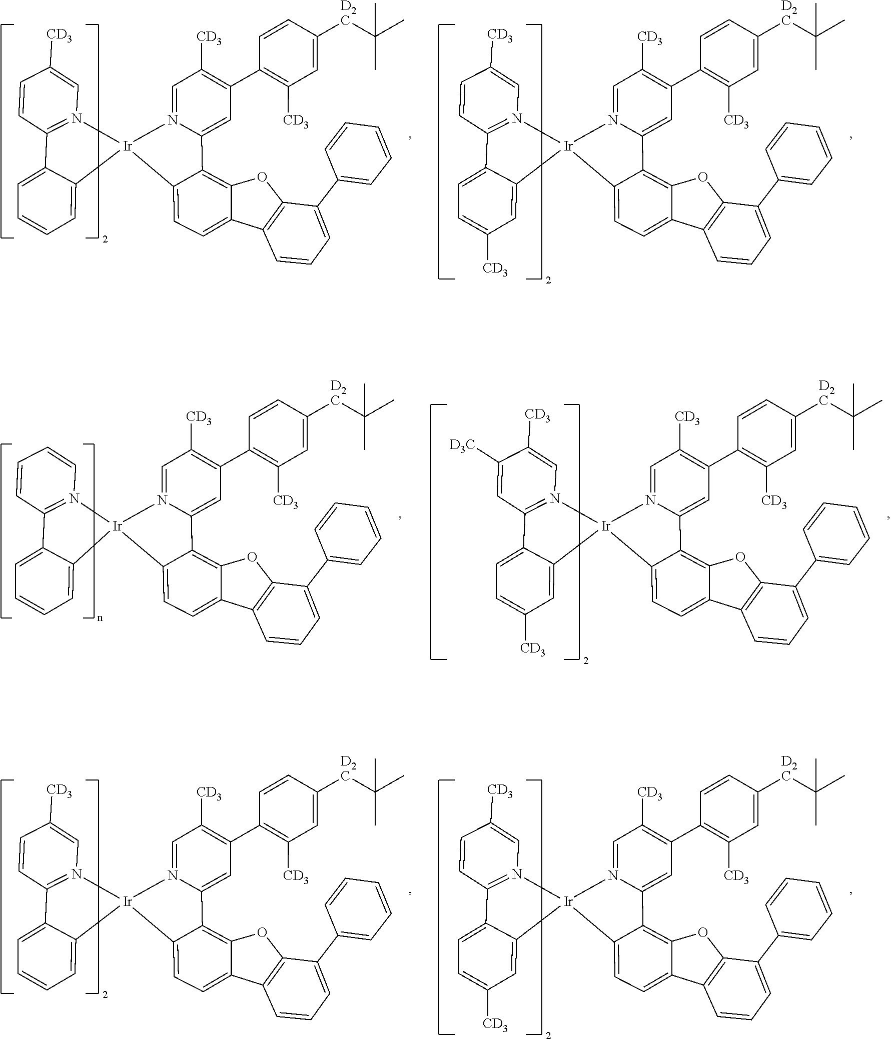

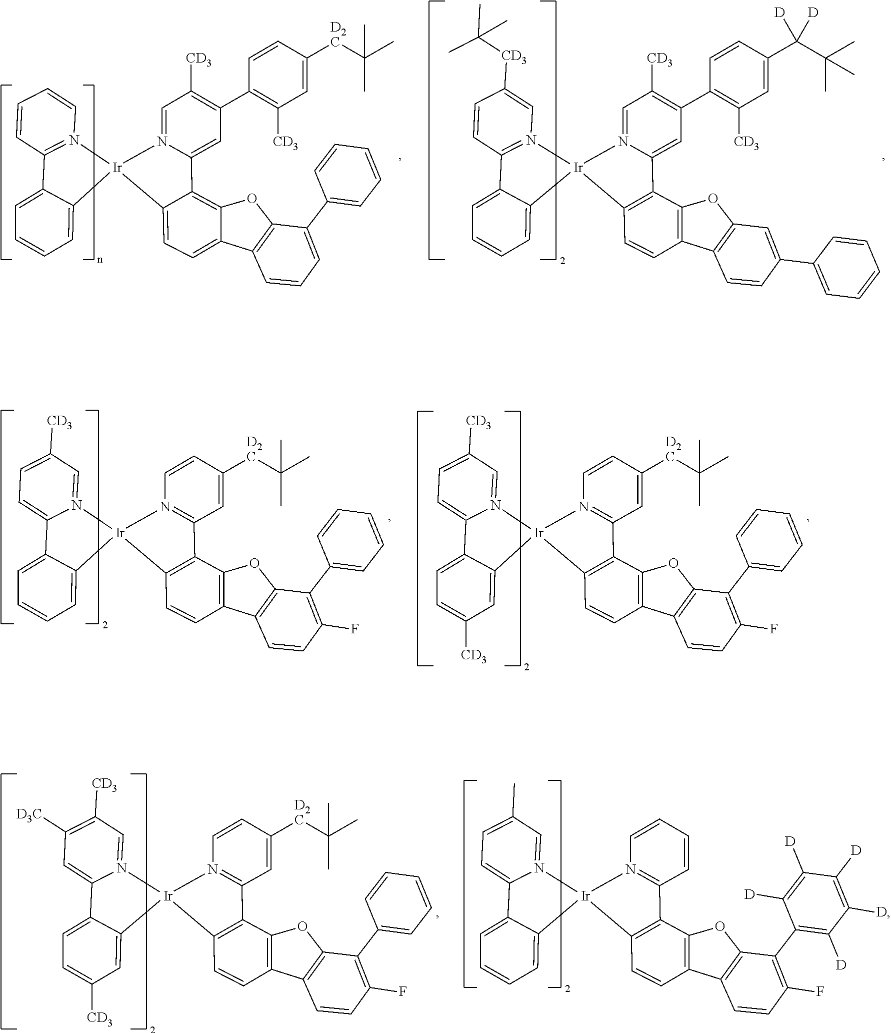

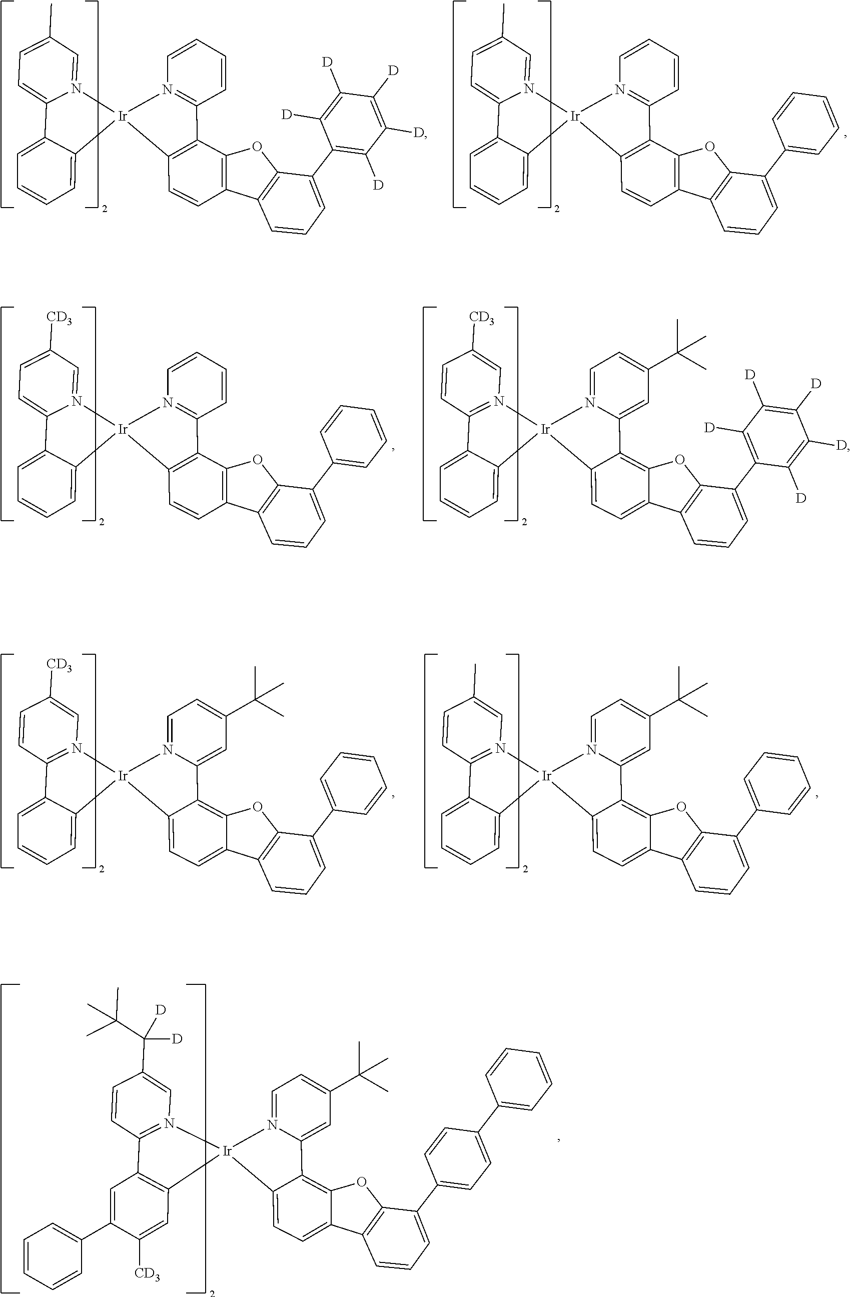

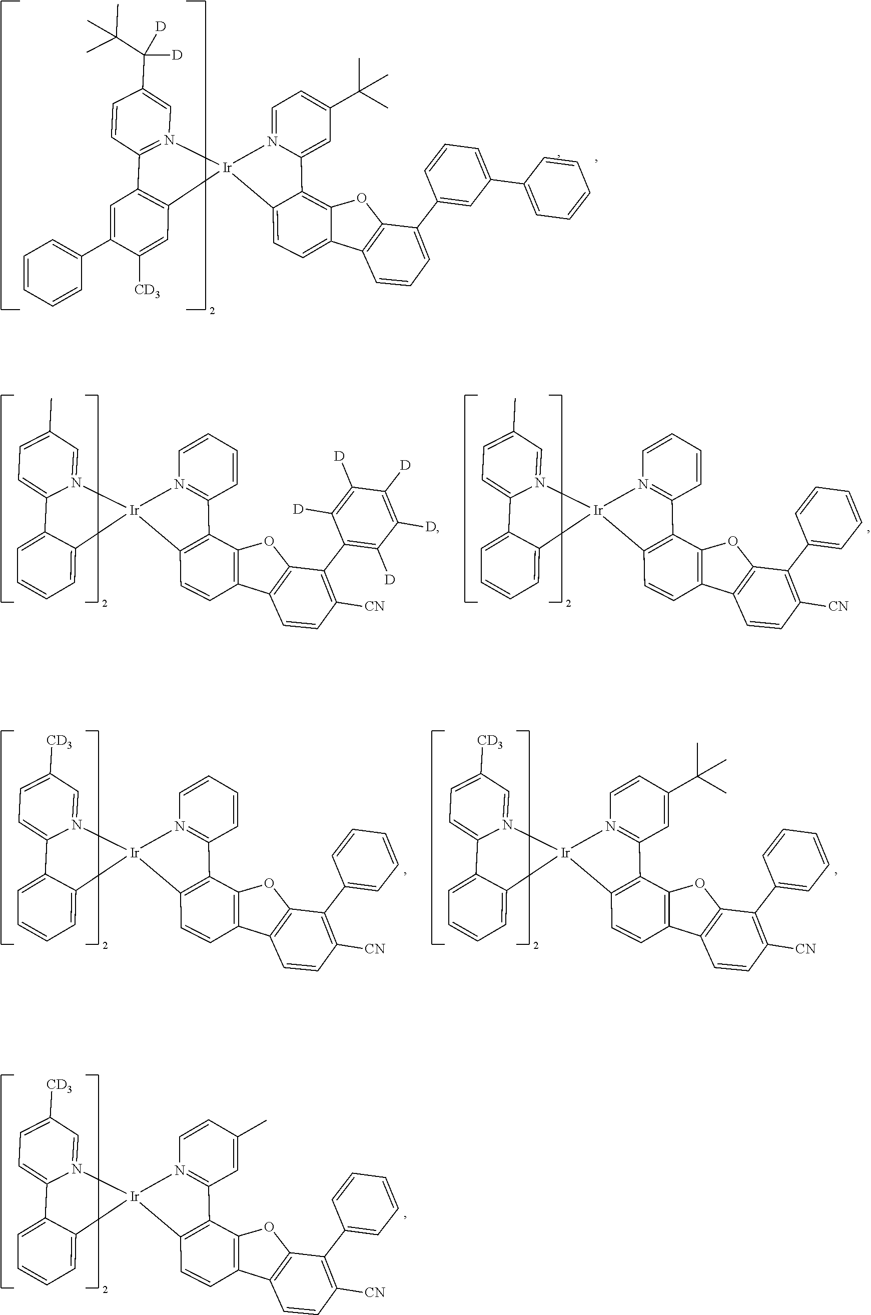

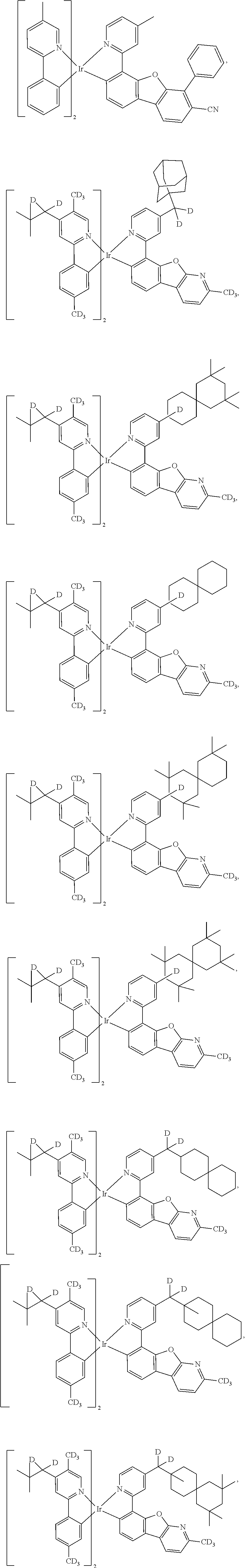

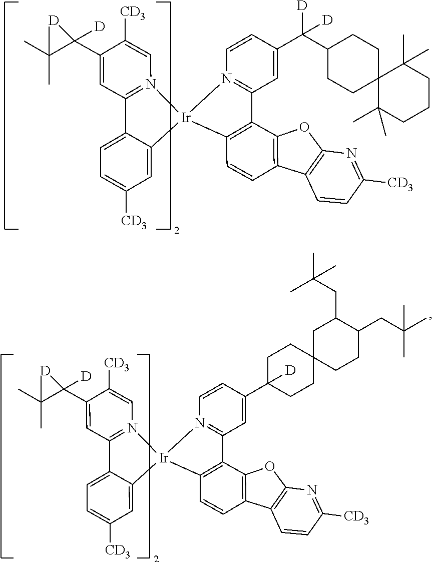

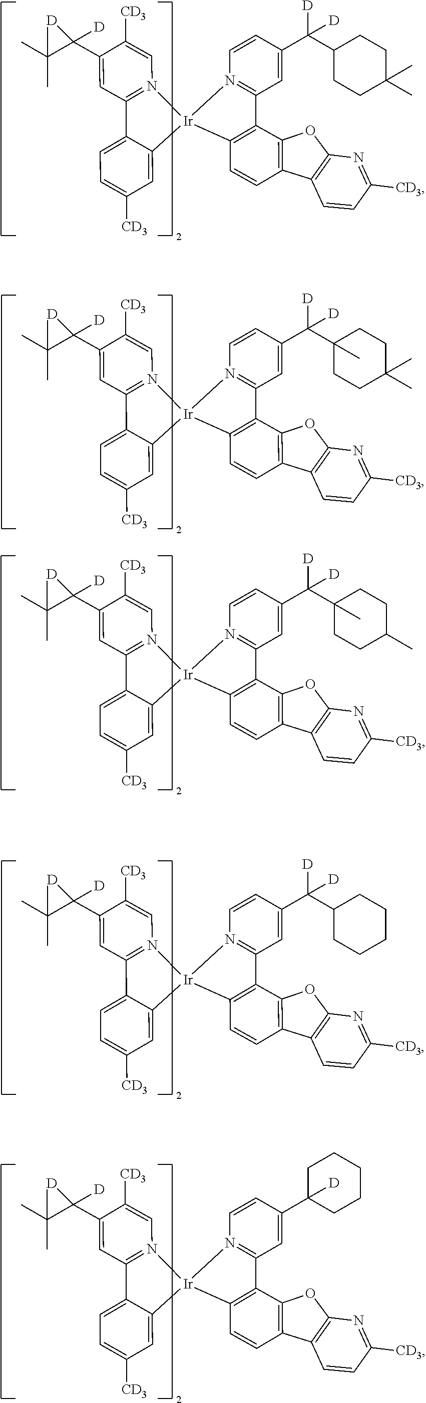

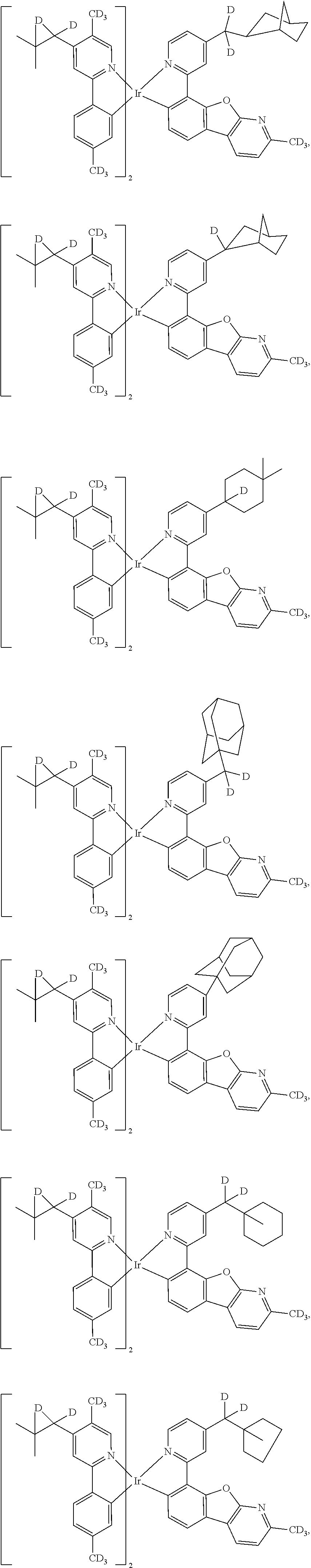

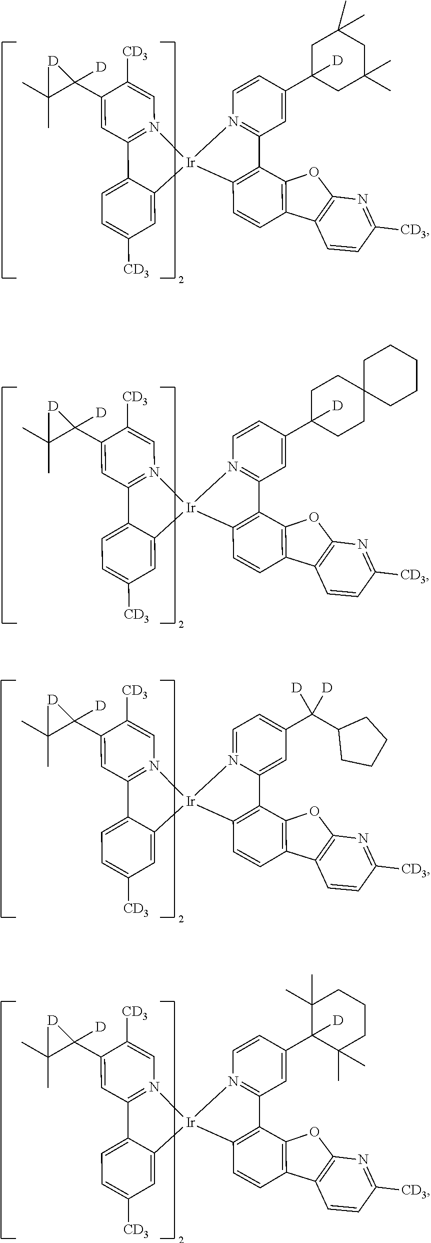

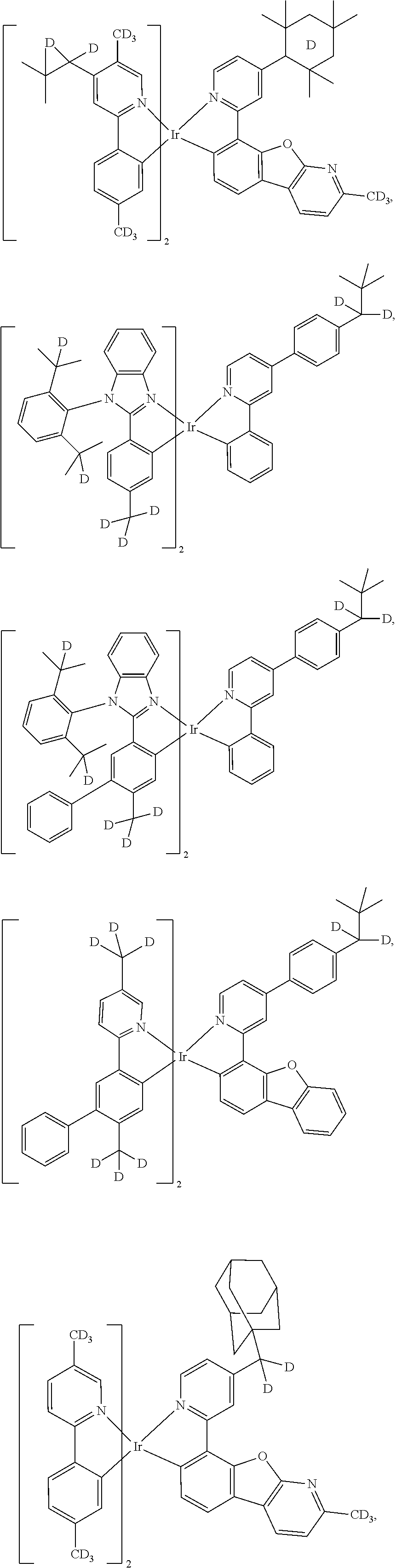

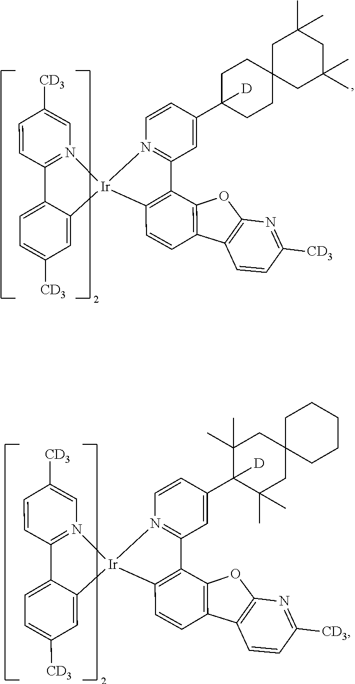

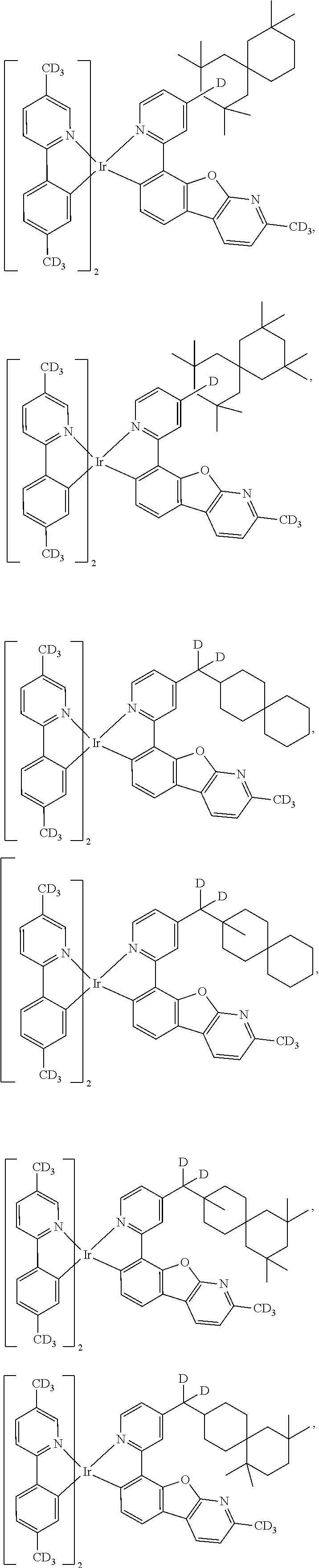

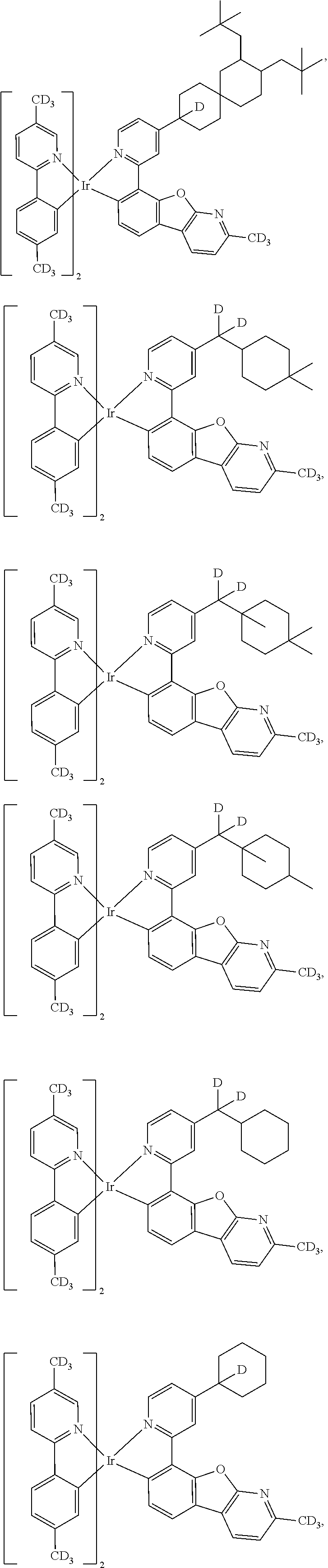

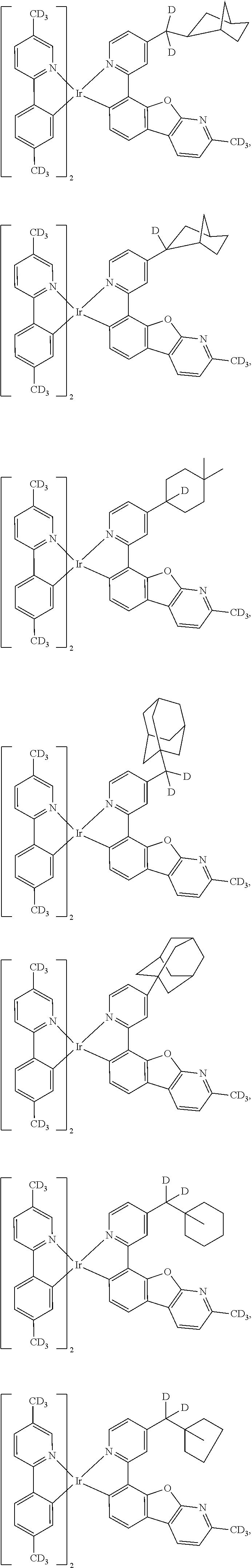

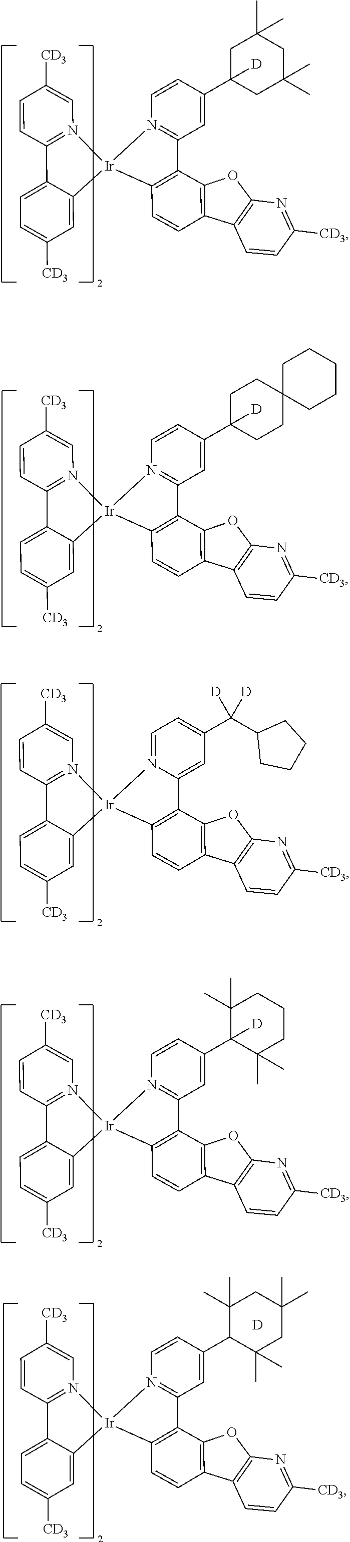

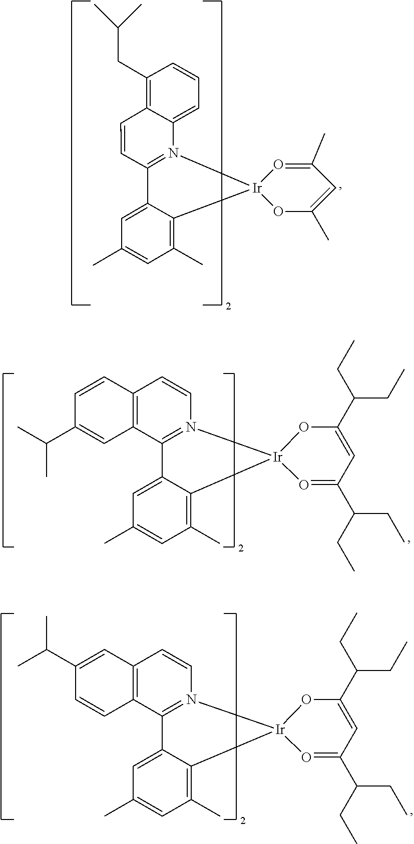

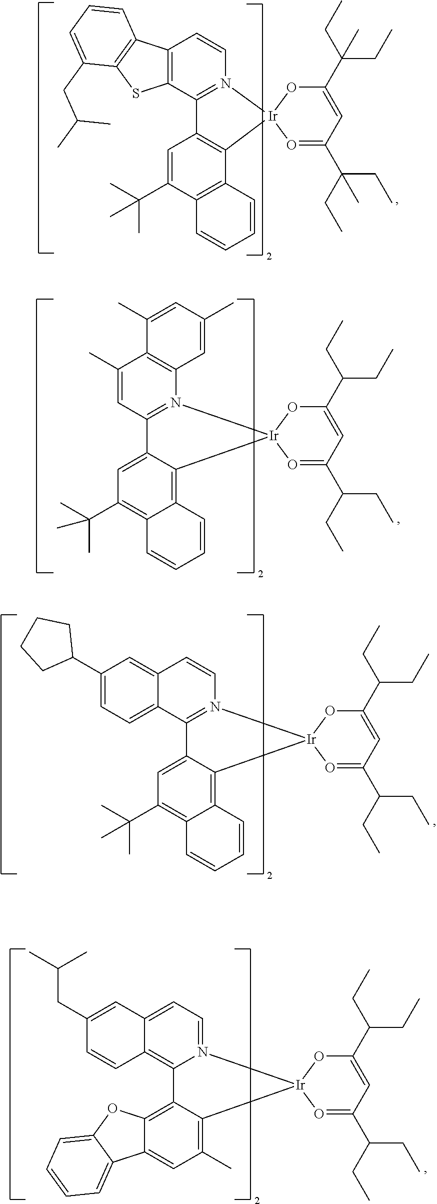

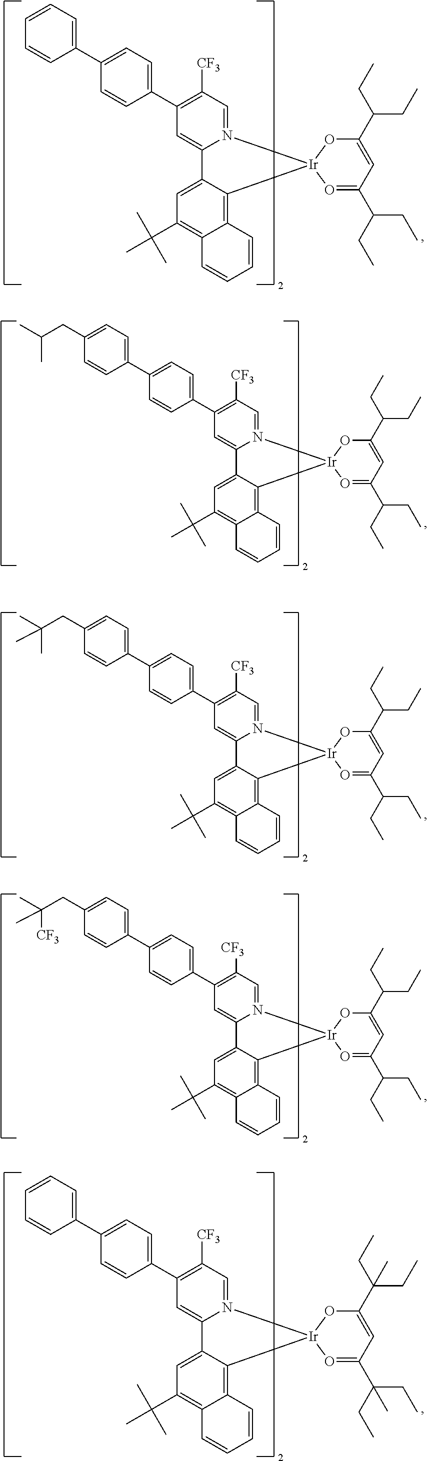

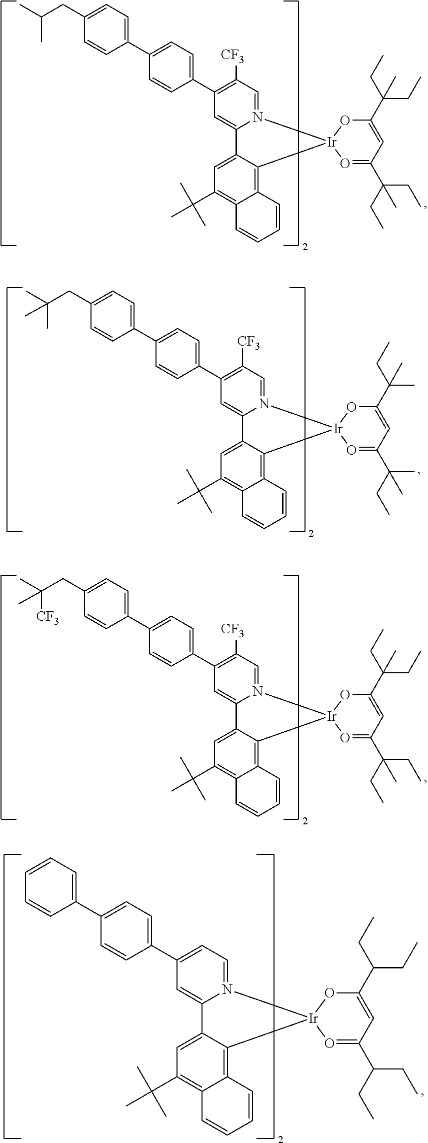

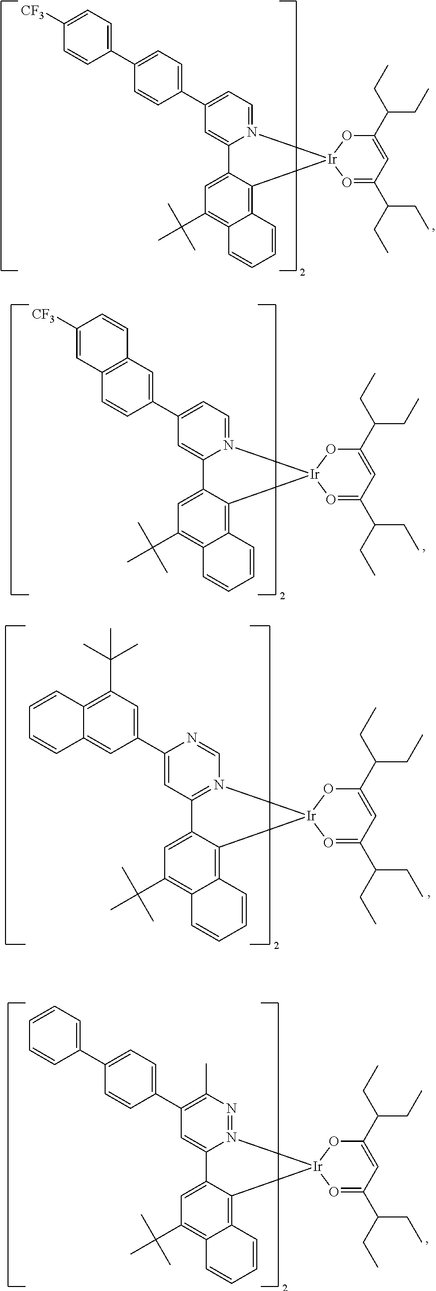

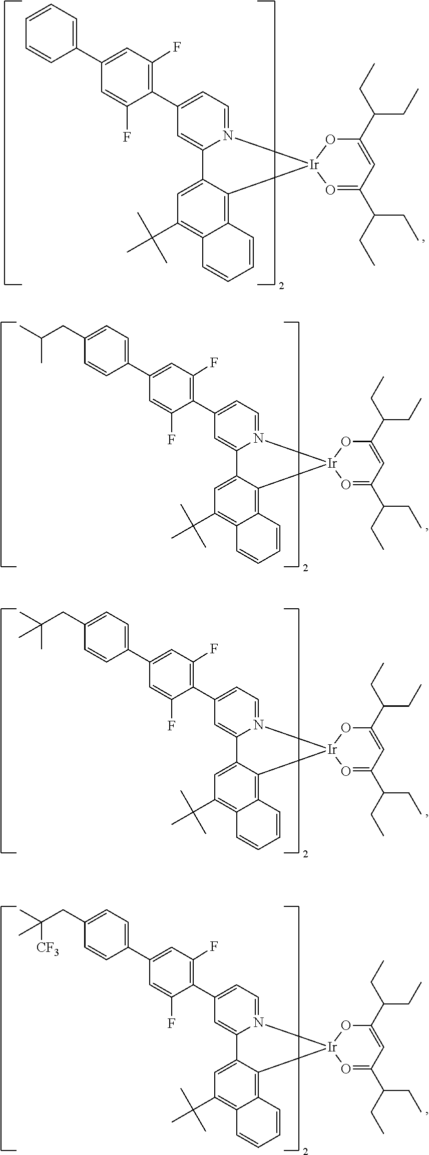

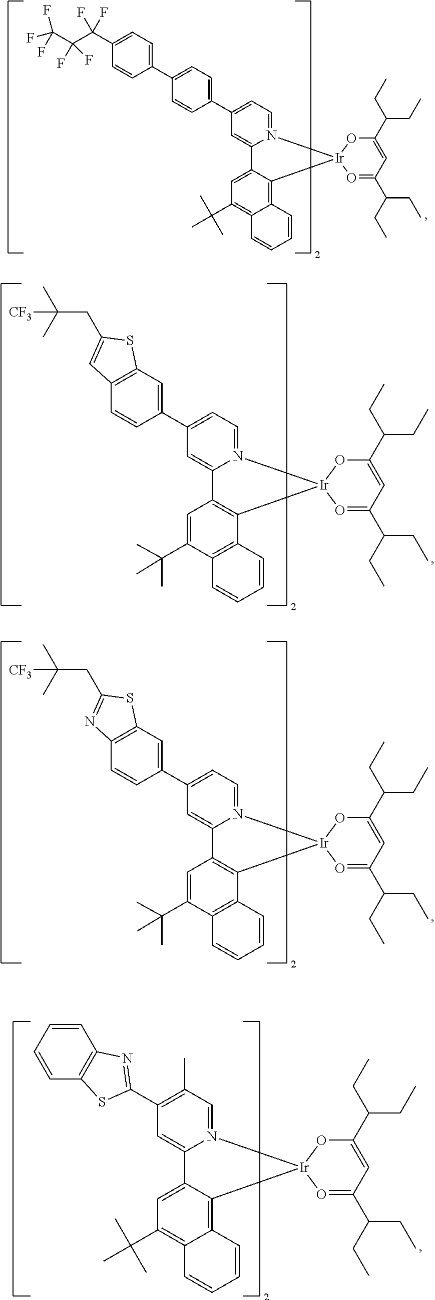

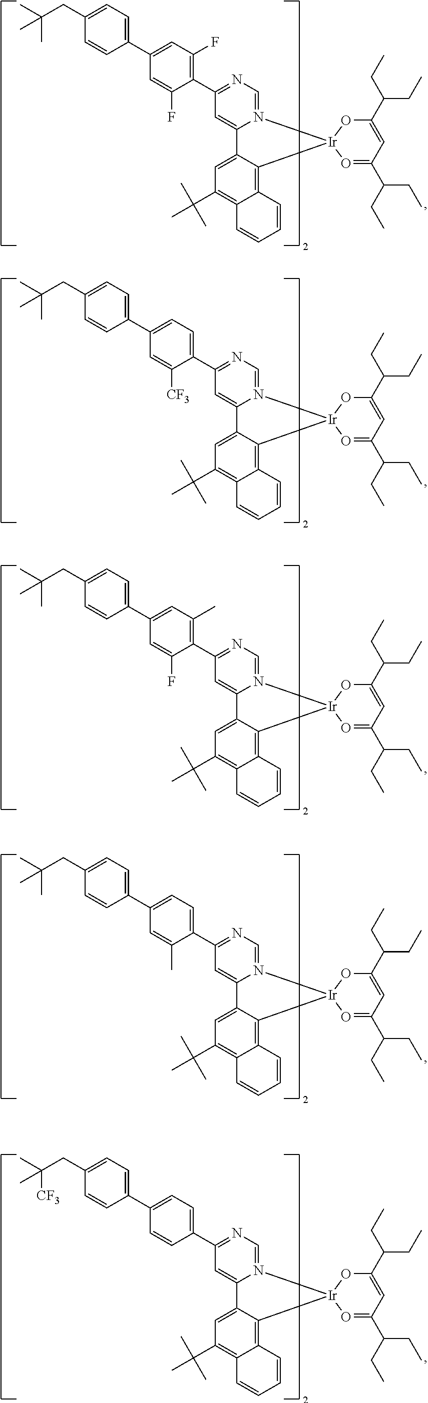

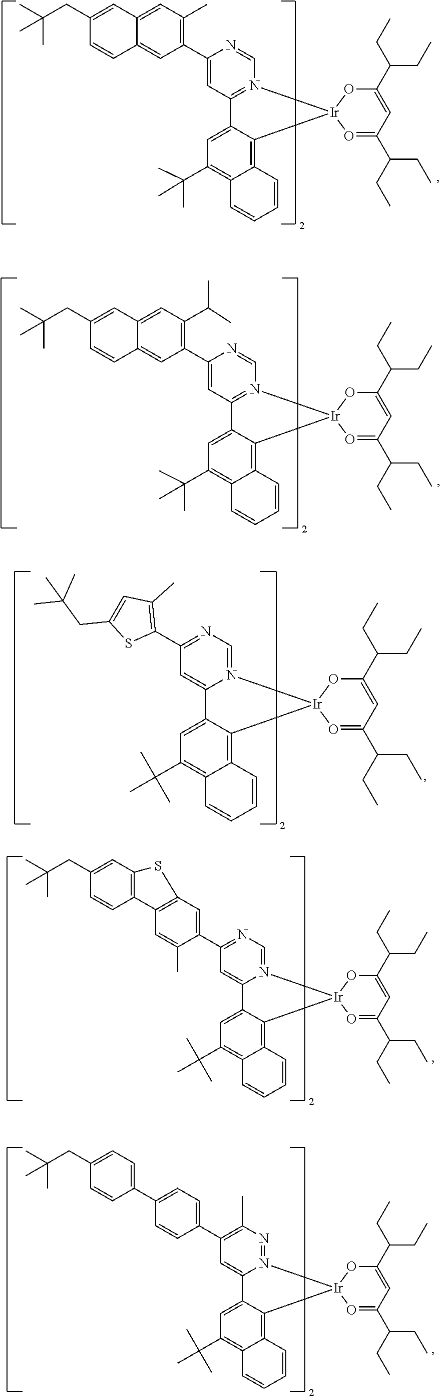

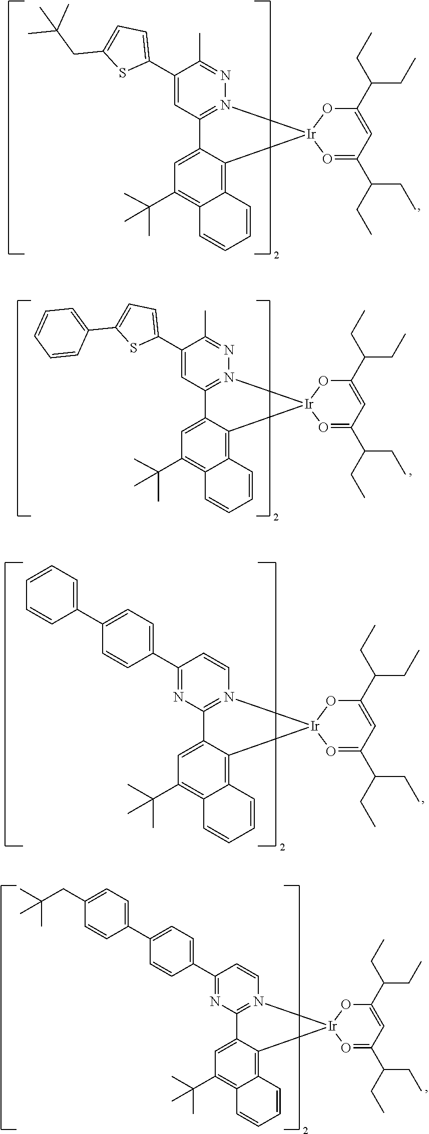

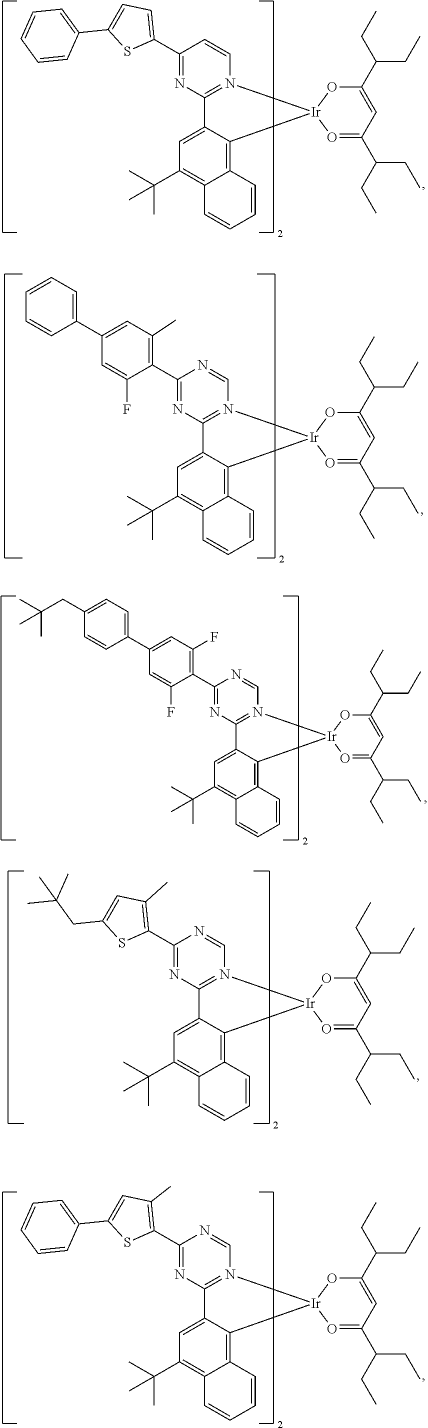

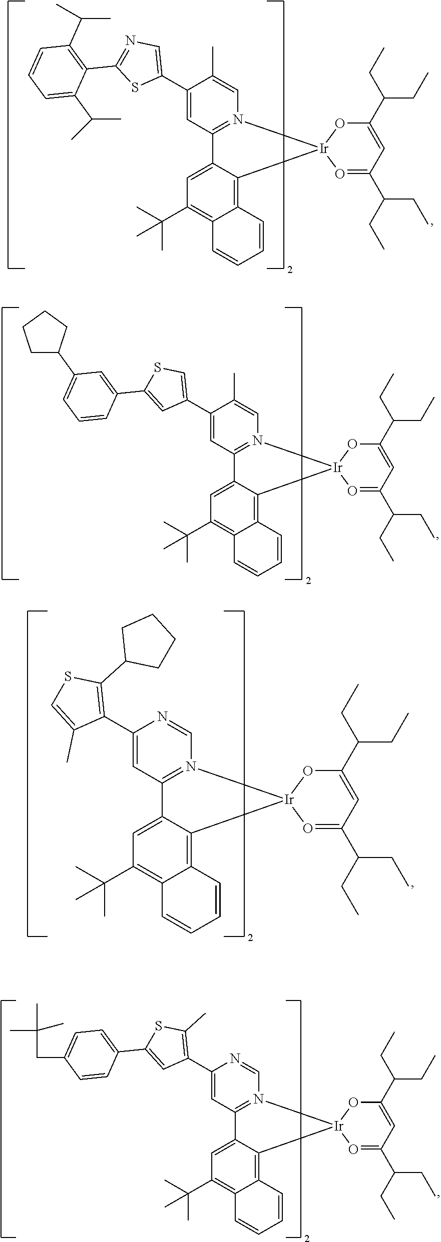

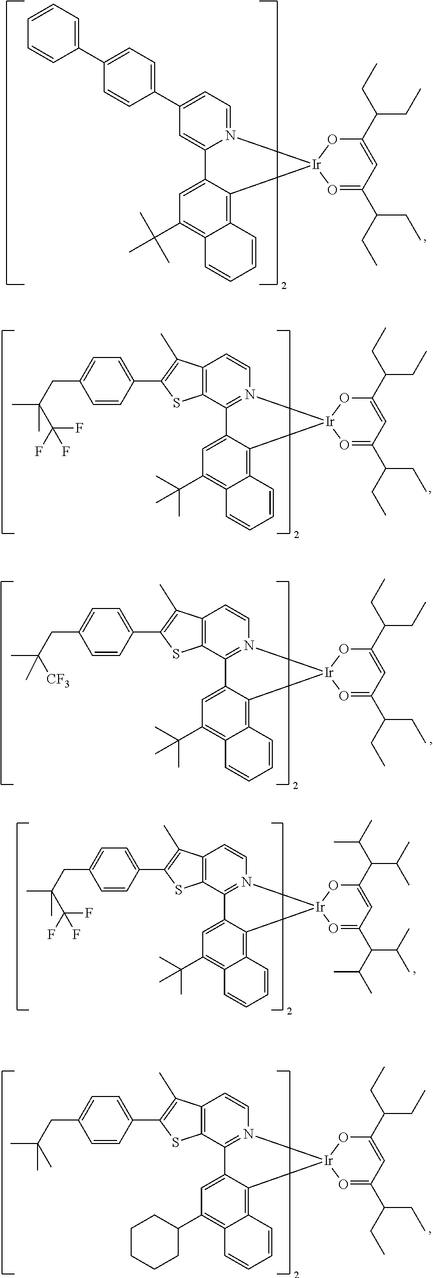

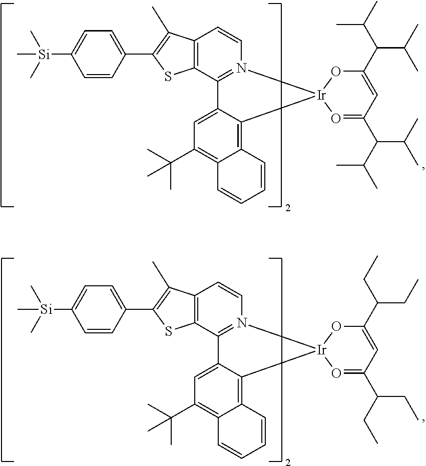

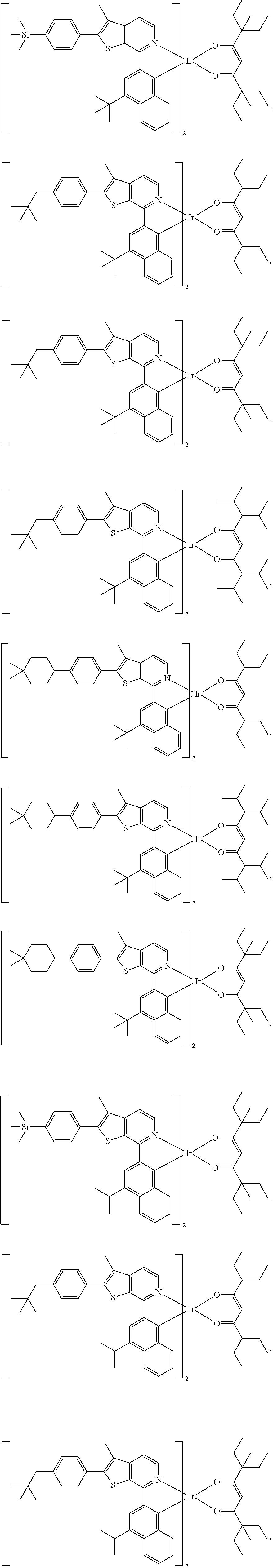

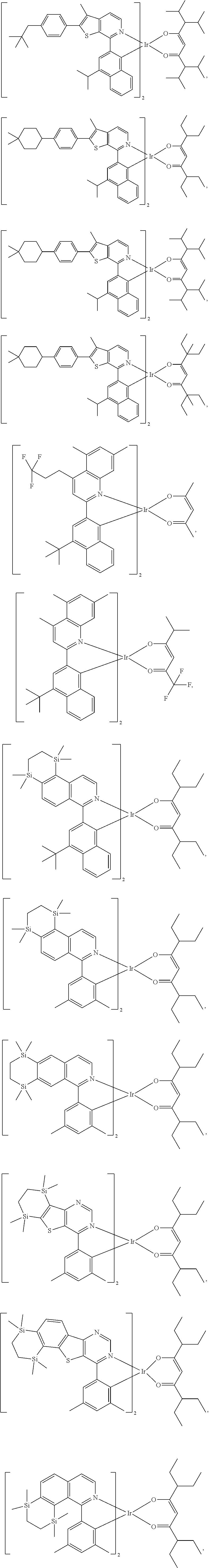

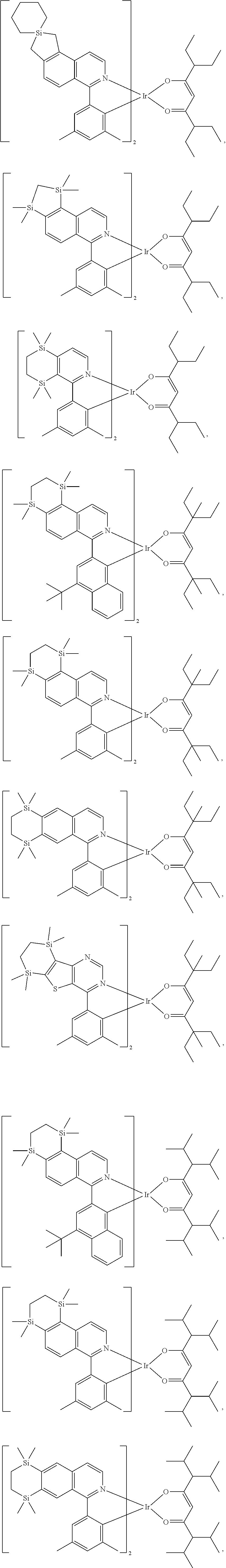

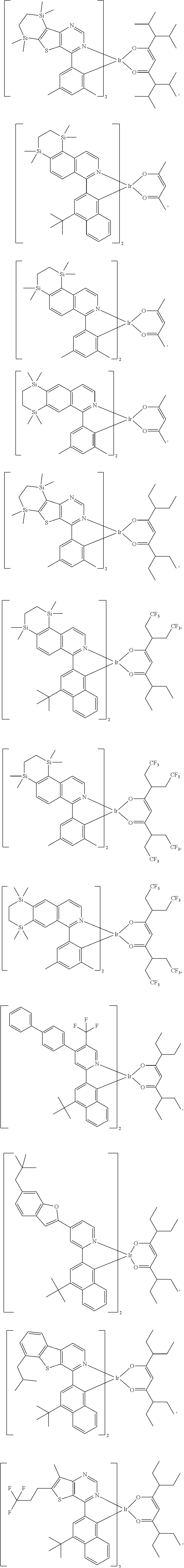

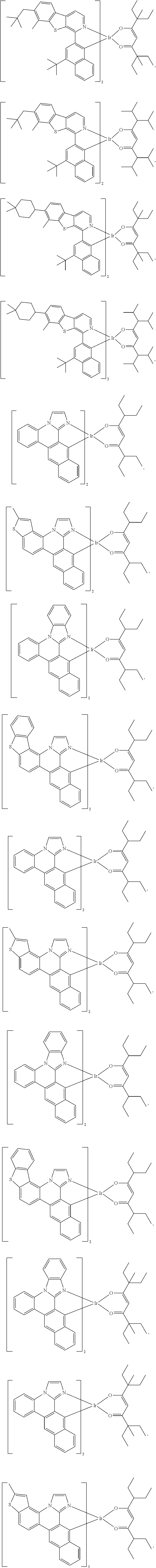

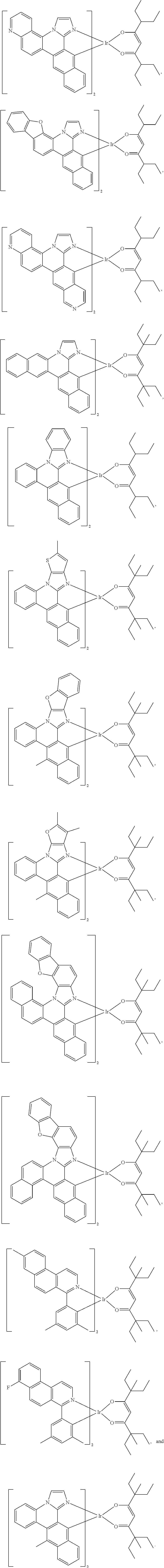

In some embodiments, the OLED has one or more characteristics selected from the group consisting of being flexible, being rollable, being foldable, being stretchable, and being curved. In some embodiments, the OLED is transparent or semi-transparent. In some embodiments, the OLED further comprises a layer comprising carbon nanotubes.

In some embodiments, the OLED further comprises a layer comprising a delayed fluorescent emitter. In some embodiments, the OLED comprises a RGB pixel arrangement or white plus color filter pixel arrangement. In some embodiments, the OLED is a mobile device, a hand held device, or a wearable device. In some embodiments, the OLED is a display panel having less than 10 inch diagonal or 50 square inch area. In some embodiments, the OLED is a display panel having at least 10 inch diagonal or 50 square inch area. In some embodiments, the OLED is a lighting panel.

In some aspects, an emissive region is provided. The emissive region can include a first compound, D1, which is a first metal complex; and a second compound, D2, which is a second metal complex, where D1 and D2 are different. The emissive region, the first compound, D1, and the second compound, D2, can be according to any embodiment described herein.

In some embodiments of the emissive region, the emissive region further comprises a host.

In some embodiments, the compound can be an emissive dopant. In some embodiments, the compound can produce emissions via phosphorescence, fluorescence, thermally activated delayed fluorescence, i.e., TADF (also referred to as E-type delayed fluorescence), triplet-triplet annihilation, or combinations of these processes.

The OLED disclosed herein can be incorporated into one or more of a consumer product, an electronic component module, and a lighting panel. The organic layer can be an emissive layer and the compound can be an emissive dopant in some embodiments, while the compound can be a non-emissive dopant in other embodiments.

The organic layer can also include a host. In some embodiments, two or more hosts are preferred. In some embodiments, the hosts used maybe a) bipolar, b) electron transporting, c) hole transporting or d) wide band gap materials that play little role in charge transport. In some embodiments, the host can include a metal complex. The host can be an inorganic compound.

### Formulations and Pre-Mixed Co-Evaporation Sources

In some aspects, formulation comprising a first compound, D1, which is a first metal complex; and a second compound, D2, which is a second metal complex, is provided. In the formulation, D1 and D2 are different; the emission a mixture of D1 and D2 does not primarily originate from D2 or from an exciplex between D1 and D2; the emission from a mixture of D1 and D2 has an emission peak maximum between 300 to 2000 nm; and if the emission from the mixture of D1 and D2 has a peak between 550 and 700 nm, then D2 is not a Be, Al, or Ir complex. The first compound, D1, and the second compound, D2, can be according to any embodiment described herein.

In another aspect, a premixed co-evaporation source that is a mixture that at least comprises a first compound (D1) and a second compound (D2) is provided. The co-evaporation source is a co-evaporation source for vacuum deposition process or organic vapor jet printing process; the first compound and the second compound are different; the first compound, D1, is a first metal complex; wherein the second compound, D2, is a second metal complex; the first compound has an evaporation temperature T1 of 150 to 400° C.; the second compound has an evaporation temperature T2 of 150 to 400° C.; an absolute value of T1−T2 is less than 20° C.; the first compound has a concentration C1 in said mixture and a concentration C2 in a film formed by evaporating the mixture in a vacuum deposition tool at a constant pressure between 1×10⁻⁶ Torr to 1×10⁻⁹ Torr, at a 2 Å/sec deposition rate on a surface positioned at a predefined distance away from the mixture being evaporated; and an absolute value of (C1−C2)/C1 is less than 5%.

In some embodiments of the premixed co-evaporation source, the first compound has evaporation temperature T1 of 150 to 400° C. and the second compound has evaporation temperature T2 of 150 to 400° C.

In some embodiments of the premixed co-evaporation source, the first compound has evaporation temperature T1 of 200 to 400° C. and the second compound has evaporation temperature T2 of 200 to 400° C.

In some embodiments of the premixed co-evaporation source, an absolute value of (C1−C2)/C1 is less than 3%.

In some embodiments of the premixed co-evaporation source, the first compound has a vapor pressure of P₁ at T1 at 1 atm, and the second compound has a vapor pressure of P₂ at T2 at 1 atm; and the ratio of P₁/P₂ is within the range of 0.90:1 to 1.10:1.

In some embodiments of the premixed co-evaporation source, D1 and D2 have a different ligand structure.

In some embodiments of the premixed co-evaporation source, D1 has a metal center M1, D2 has a metal center M2, and M1 and M2 are the same.

In some embodiments of the premixed co-evaporation source, D1 has a metal center M1, D2 has a metal center M2, and M1 and M2 are different.

In some embodiments of the premixed co-evaporation source, M2 is selected from the group consisting of Ir, Pt, Pd, Au, Ag, and Cu. In some embodiments of the premixed co-evaporation source, M2 is Pd.

In some embodiments of the premixed co-evaporation source, D1 has at least one ligand with the same structure as a ligand in D2.

In some embodiments of the premixed co-evaporation source, D1 and D2 have the same ligand structure.

In some embodiments of the premixed co-evaporation source, the first compound has a first mass loss rate and the second compound has a second mass loss rate, wherein the ratio between the first mass loss rate and the second mass loss rate is within the range of 0.90:1 to 1.10:1. In some such embodiments, the ratio between the first mass loss rate and the second mass loss rate is within the range of 0.95:1 to 1.05:1. In some such embodiments, the ratio between the first mass loss rate and the second mass loss rate is within the range of 0.97:1 to 1.03:1.

Combination with Other Materials

The materials described herein are as various examples useful for a particular layer in an OLED. They may also be used in combination with a wide variety of other materials present in the device. For example, emissive dopants disclosed herein may be used by themselves in the EML, or in conjunction with a wide variety of other emitters, hosts, transport layers, blocking layers, injection layers, electrodes and other layers that may be present. The materials described or referred to below are non-limiting examples of materials that may be useful in combination with the compounds and the devices disclosed herein, and one of skill in the art can readily consult the literature to identify other materials that may be useful in combination.

### a) Conductivity Dopants:

A charge transport layer can be doped with conductivity dopants to substantially alter its density of charge carriers, which will in turn alter its conductivity. The conductivity is increased by generating charge carriers in the matrix material, and depending on the type of dopant, a change in the Fermi level of the semiconductor may also be achieved. Hole-transporting layer can be doped by p-type conductivity dopants and n-type conductivity dopants are used in the electron-transporting layer. In some embodiments, conductivity dopants comprise at least one chemical moiety selected from the group consisting of cyano, fluorinated aryl or heteroaryl, fluorinated alkyl or cycloalkyl, alkylene, heteroaryl, amide, benzodithiophene, and highly conjugated heteroaryl groups extended by non-ring double bonds.

### b) HIL/HTL:

A hole injecting/transporting material to be used in the present disclosure is not particularly limited, and any compound may be used as long as the compound is typically used as a hole injecting/transporting material. Examples of the material include, but are not limited to: a phthalocyanine or porphyrin derivative; an aromatic amine derivative; an indolocarbazole derivative; a polymer containing fluorohydrocarbon; a polymer with conductivity dopants; a conducting polymer, such as PEDOT/PSS; a self-assembly monomer derived from compounds such as phosphonic acid and silane derivatives; a metal oxide derivative, such as MoOX; a p-type semiconducting organic compound, such as 1,4,5,8,9,12-Hexaazatriphenylenehexacarbonitrile; a metal complex, and a cross-linkable compounds.

Examples of aromatic amine derivatives used in HIL or HTL include, but not limit to the following general structures:

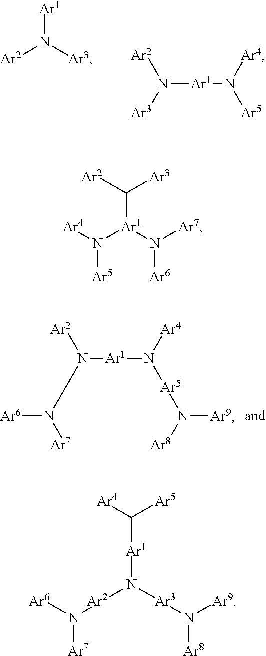

Each of Ar¹ to Ar⁹ is selected from the group consisting of aromatic hydrocarbon cyclic compounds such as benzene, biphenyl, triphenyl, triphenylene, naphthalene, anthracene, phenalene, phenanthrene, fluorene, pyrene, chrysene, perylene, and azulene; the group consisting of aromatic heterocyclic compounds such as dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, and selenophenodipyridine; and the group consisting of 2 to 10 cyclic structural units which are groups of the same type or different types selected from the aromatic hydrocarbon cyclic group and the aromatic heterocyclic group and are bonded to each other directly or via at least one of oxygen atom, nitrogen atom, sulfur atom, silicon atom, phosphorus atom, boron atom, chain structural unit and the aliphatic cyclic group. Each of Ar¹ to Ar⁹ may be unsubstituted or may be substituted by a general substituent as described above, any two substituents can be joined or fused into a ring.

In some embodiments, each Ar¹ to Ar⁹ independently comprises a moiety selected from the group consisting of:

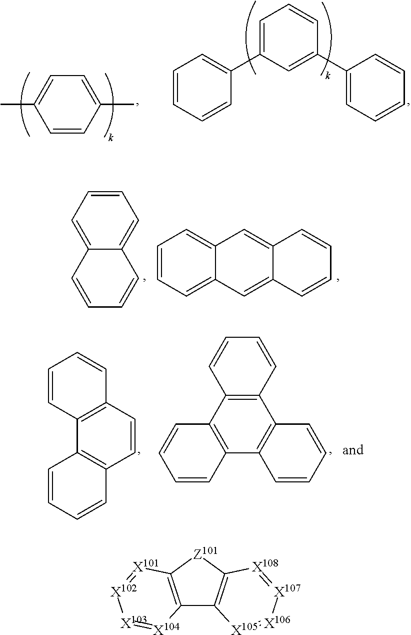

wherein k is an integer from 1 to 20; X¹⁰¹ to X¹⁰⁸ is C or N; Z¹⁰¹ is C, N, O, or S.

Examples of metal complexes used in HIL or HTL include, but are not limited to the following general formula:

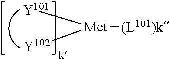

wherein Met is a metal, which can have an atomic weight greater than 40; (Y¹⁰¹-Y¹⁰²) is a bidentate ligand, the coordinating atoms of Y¹⁰¹ and Y¹⁰² are independently selected from C, N, O, P, and S; L¹⁰¹ is an another ligand; k′ is an integer value from 1 to the maximum number of ligands that may be attached to the metal; and k′+k″ is the maximum number of ligands that may be attached to the metal.

In some embodiments, (Y¹⁰¹-Y¹⁰²) is a 2-phenylpyridine or 2-phenylimidazole derivative. In some embodiments, (Y¹⁰¹-Y¹⁰²) is a carbene ligand. In some embodiments, Met is selected from Ir, Pt, Pd, Os, Cu, and Zn. In some embodiments, the metal complex has a smallest oxidation potential in solution vs. Fc⁺/Fc couple less than about 0.6 V.

In some embodiments, the HIL/HTL material is selected from the group consisting of phthalocyanine and porphryin compounds, starburst triarylamines, CFX fluorohydrocarbon polymer, conducting polymers (e.g., PEDOT:PSS, polyaniline, polypthiophene), phosphonic acid and silane SAMs, triarylamine or polythiophene polymers with conductivity dopants, Organic compounds with conductive inorganic compounds (such as molybdenum and tungsten oxides), n-type semiconducting organic complexes, metal organometallic complexes, cross-linkable compounds, polythiophene based polymers and copolymers, triarylamines, triaylamine with spirofluorene core, arylamine carbazole compounds, triarylamine with (di)benzothiophene/(di)benzofuran, indolocarbazoles, isoindole compounds, and metal carbene complexes.

### c) EBL:

An electron blocking layer (EBL) may be used to reduce the number of electrons and/or excitons that leave the emissive layer. The presence of such a blocking layer in a device may result in substantially higher efficiencies, and/or longer lifetime, as compared to a similar device lacking a blocking layer. Also, a blocking layer may be used to confine emission to a desired region of an OLED. In some embodiments, the EBL material has a higher LUMO (closer to the vacuum level) and/or higher triplet energy than one or more emitters closest to the EBL interface. In some embodiments, the compound used in EBL contains at least one carbazole group and/or at least one arylamine group. In some embodiments the HOMO level of the compound used in the EBL is shallower than the HOMO level of one or more of the hosts in the EML. In some embodiments, the compound used in EBL contains the same molecule or the same functional groups used as one of the hosts described herein.

### d) Hosts:

The light emitting layer of the organic EL device of the present disclosure preferably contains at least a light emitting material as the dopant, and a host material. Examples of the host material are not particularly limited, and any metal complexes or organic compounds may be used as long as the host won't fully quench the emission of the dopant.

Examples of metal complexes used as host are preferred to have the following general formula:

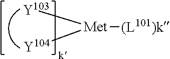

wherein Met is a metal; (Y¹⁰³-Y¹⁰⁴) is a bidentate ligand, the coordinating atoms of Y¹⁰³ and Y¹⁰⁴ are independently selected from C, N, O, P, and S; L¹⁰¹ is an another ligand; k′ is an integer value from 1 to the maximum number of ligands that may be attached to the metal; and k′+k″ is the maximum number of ligands that may be attached to the metal.

In some embodiments, the metal complexes are:

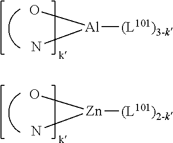

wherein (O—N) is a bidentate ligand, having metal coordinated to atoms O and N.

In some embodiments, Met is selected from Ir and Pt. In a further embodiments, (Y¹⁰³-Y¹⁰⁴) is a carbene ligand.

In some embodiments, the host compound contains at least one of the following groups selected from the group consisting of aromatic hydrocarbon cyclic compounds such as benzene, biphenyl, triphenyl, triphenylene, tetraphenylene, naphthalene, anthracene, phenalene, phenanthrene, fluorene, pyrene, chrysene, perylene, and azulene; the group consisting of aromatic heterocyclic compounds such as dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, aza-dibenzothiophene, aza-dibenzofuran, aza-dibenzoselenophene, aza-carbazole, aza-indolocarbazole, aza-triphenylene, aza-tetraphenylene, 5λ²-benzo[d]benzo[4,5]imidazo[3,2-a]imidazole, 5,9-dioxa-13b-boranaphtho[3,2,1-de]anthracene; and the group consisting of 2 to 10 cyclic structural units which are groups of the same type or different types selected from the aromatic hydrocarbon cyclic group and the aromatic heterocyclic group and are bonded to each other directly or via at least one of oxygen atom, nitrogen atom, sulfur atom, silicon atom, phosphorus atom, boron atom, chain structural unit and the aliphatic cyclic group. Each option within each group may be unsubstituted or may be substituted by the general substituents as described herein or may be further fused.

In some embodiments, the host compound comprises at least one of the moieties selected from the group consisting of:

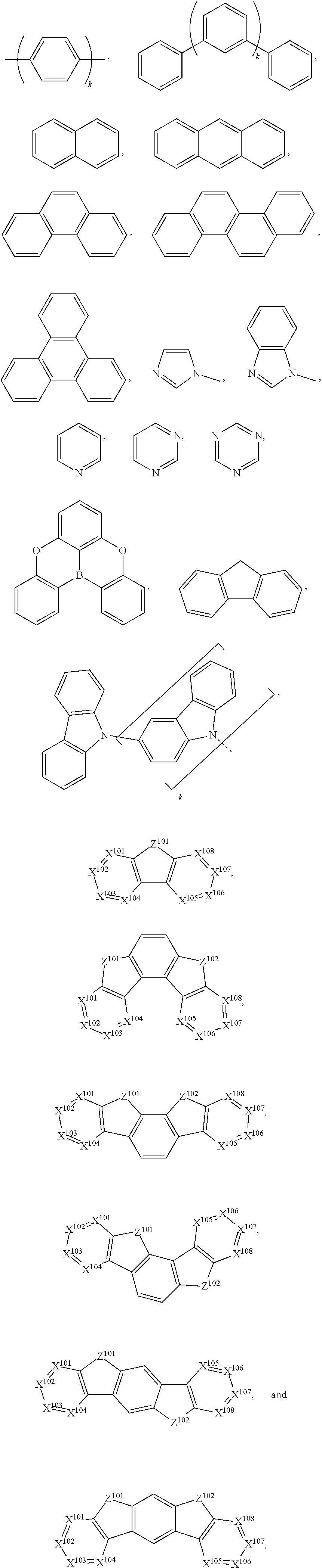

wherein k is an integer from 0 to 20 or 1 to 20. X¹⁰¹ to X¹⁰⁸ are independently selected from C or N. Z¹⁰¹ and Z¹⁰² are independently selected from C, N, O, or S.

In some embodiments, the host material is selected from the group consisting of arylcarbazoles, metal 8-hydroxyquinolates, (e.g., alq3, balq), metal phenoxybenzothiazole compounds, conjugated oligomers and polymers (e.g., polyfluorene), aromatic fused rings, zinc complexes, chrysene based compounds, aryltriphenylene compounds, poly-fused heteroaryl compounds, donor acceptor type molecules, dibenzofuran/dibenzothiophene compounds, polymers (e.g., pvk), spirofluorene compounds, spirofluorene-carbazole compounds, indolocabazoles, 5-member ring electron deficient heterocycles (e.g., triazole, oxadiazole), tetraphenylene complexes, metal phenoxypyridine compounds, metal coordination complexes (e.g., Zn, Al with N\hat{ }N ligands), dibenzothiophene/dibenzofuran-carbazole compounds, silicon/germanium aryl compounds, aryl benzoyl esters, carbazole linked by non-conjugated groups, aza-carbazole/dibenzofuran/dibenzothiophene compounds, and high triplet metal organometallic complexes (e.g., metal-carbene complexes).

### e) Emitter Materials in EML:

One or more emitter materials may be used in conjunction with the compound or device of the present disclosure. The emitter material can be emissive or non-emissive in the current device as described herein. Examples of the emitter materials are not particularly limited, and any compounds may be used as long as the compounds are capable of producing emissions in a regular OLED device. Examples of suitable emitter materials include, but are not limited to, compounds which are capable of producing emissions via phosphorescence, non-delayed fluorescence, delayed fluorescence, especially the thermally activated delayed fluorescence, i.e., TADF (also referred to as E-type delayed fluorescence), triplet-triplet annihilation, or combinations of these processes.

In some embodiments, the emitter material has the formula of M(L¹)ₓ(L²)_{y}(L³)_{z};
wherein L¹, L², and L³ can be the same or different;
wherein x is 1, 2, or 3;
wherein y is 0, 1, or 2;
wherein z is 0, 1, or 2;
wherein x+y+z is the oxidation state of the metal M;
wherein L¹ is selected from the group consisting of the structures of LIST 1 defined herein wherein each L² and L³ are independently selected from the group consisting of

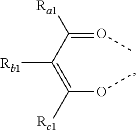

and the structures of LIST 4; wherein:
M is selected from the group consisting of Ir, Rh, Re, Ru, Os, Pt, Pd, Zn, Au, Ag, and Cu;
T is selected from the group consisting of B, Al, Ga, and In;
K^{1′} is a direct bond or is selected from the group consisting of NRₑ, PRₑ, O, S, and Se;
each Y¹ to Y⁵ are independently selected from the group consisting of carbon and nitrogen;
Y′ is selected from the group consisting of BRₑ, NRₑ, PRₑ, O, S, Se, C═O, S═O, SO₂, CRₑR_{f}, SiRₑR_{f}, and GeRₑR_{f};
each Rₐ, R_{b}, R_{c}, and R_{d} can independently represent from mono to the maximum possible number of substitutions, or no substitution;
each Rₐ₁, R_{b1}, R_{c1}, R_{d1}, Rₐ, R_{b}, R_{c}, R_{d}, Rₑ, and R_{f} is independently a hydrogen or a substituent selected from the group consisting of the general substituents as defined herein; and
wherein any two substituents can be fused or joined to form a ring or form a multidentate ligand.

In some embodiments, the emitter material is selected from the group consisting of the structures of LIST 5 as defined herein.

In some embodiments of the LIST 2, each unsubstituted aromatic carbon atom can be replaced with N to form an aza-ring. In some embodiments, the maximum number of N atom in one ring is 1 or 2.

In some embodiments of the OLED, the delayed fluorescence material comprises at least one donor group and at least one acceptor group. In some embodiments, the delayed fluorescence material is a metal complex. In some embodiments, the delayed fluorescence material is a non-metal complex. In some embodiments, the delayed fluorescence material is a Zn, Cu, Ag, or Au complex.

In some embodiments of the OLED, the delayed fluorescence material has the formula of M(L⁵)(L⁶), wherein M is Cu, Ag, or Au, L⁵ and L⁶ are different, and L⁵ and L⁶ are independently selected from the group consisting of:

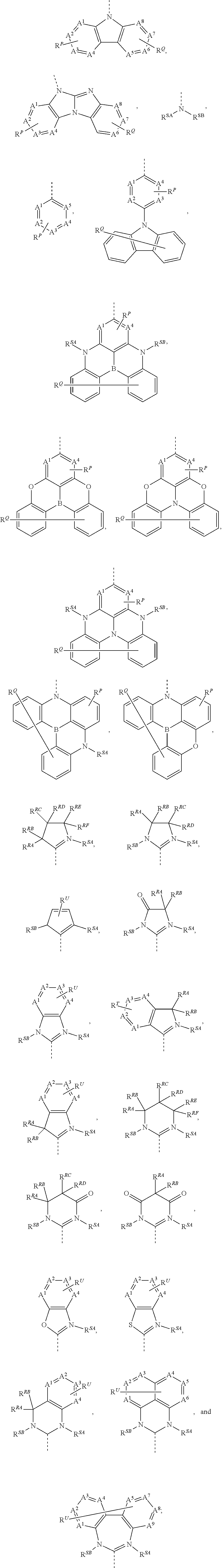

wherein A¹-A⁹ are each independently selected from C or N;
each R^{P}, R^{Q}, and R^{U} independently represents mono-, up to the maximum substitutions, or no substitutions;
wherein each R^{P}, R^{P}, R^{U}, R^{SA}, R^{SB}, R^{RA}, R^{RB}, R^{RC}, R^{RD}, R^{RE}, and R^{RF} is independently a hydrogen or a substituent selected from the group consisting of the general substituents as defined herein; any two substituents can be joined or fused to form a ring.

In some embodiments of the OLED, the delayed fluorescence material comprises at least one of the donor moieties selected from the group consisting of:

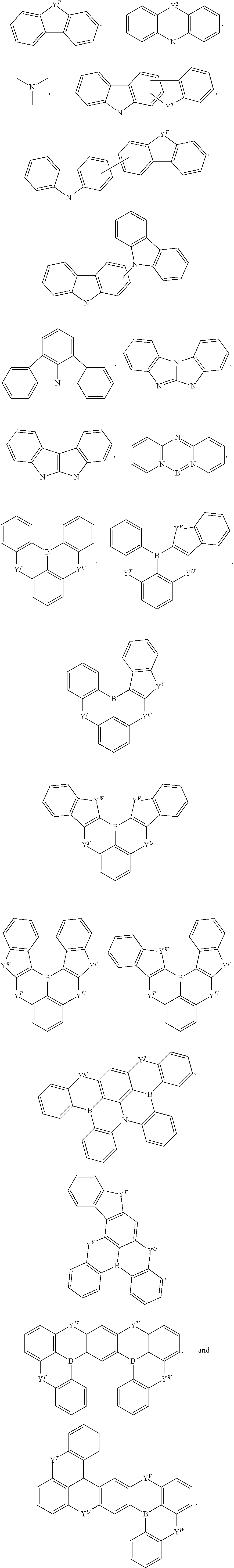

wherein Y^{T}, Y^{U}, Y^{V}, and Y^{W} are each independently selected from the group consisting of B, C, Si, Ge, N, P, O, S, Se, C═O, S═O, and SO₂.

In some of the above embodiments, any carbon ring atoms up to maximum of a total number of three, together with their substituents, in each phenyl ring of any of above structures can be replaced with N.

In some embodiments, the delayed fluorescence material comprises at least one of the acceptor moieties selected from the group consisting of nitrile, isonitrile, borane, fluoride, pyridine, pyrimidine, pyrazine, triazine, aza-carbazole, aza-dibenzothiophene, aza-dibenzofuran, aza-dibenzoselenophene, aza-triphenylene, imidazole, pyrazole, oxazole, thiazole, isoxazole, isothiazole, triazole, thiadiazole, and oxadiazole. In some embodiments, the acceptor moieties and the donor moieties as described herein can be connected directly, through a conjugated linker, or a non-conjugated linker, such as a sp³ carbon or silicon atom.

In some embodiments, the fluorescent material comprises at least one of the chemical moieties selected from the group consisting of:

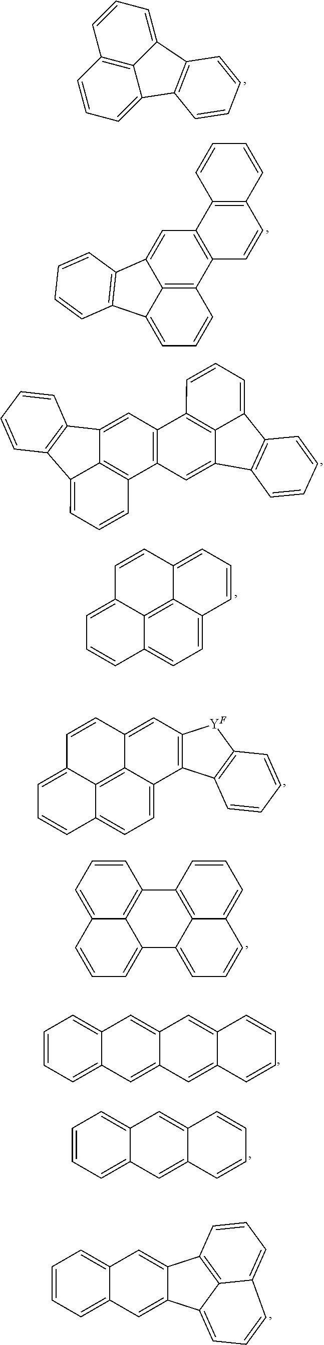

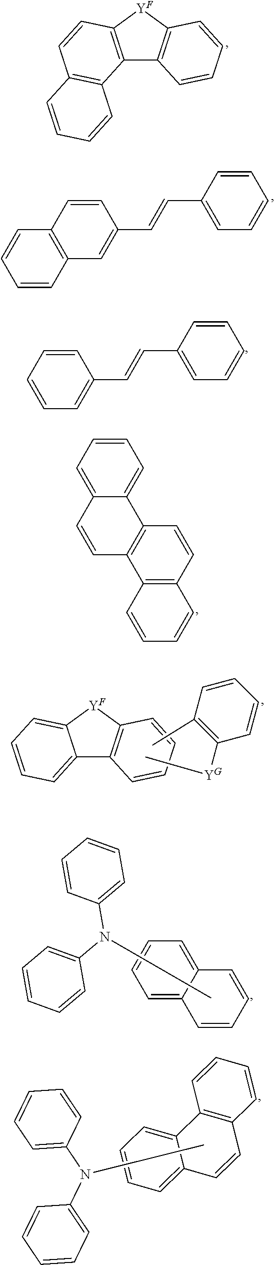

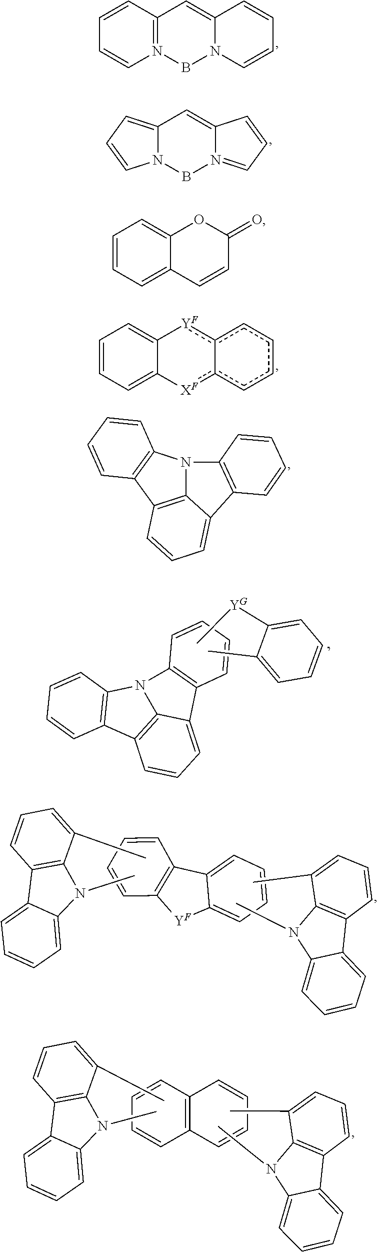

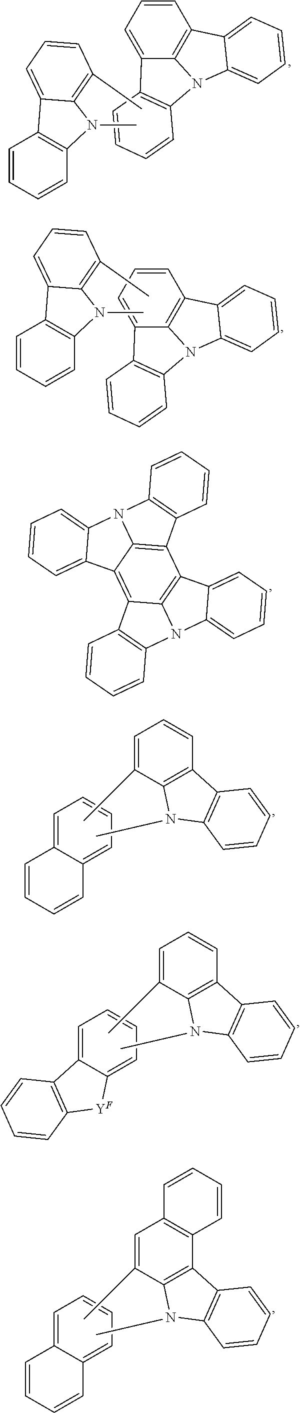

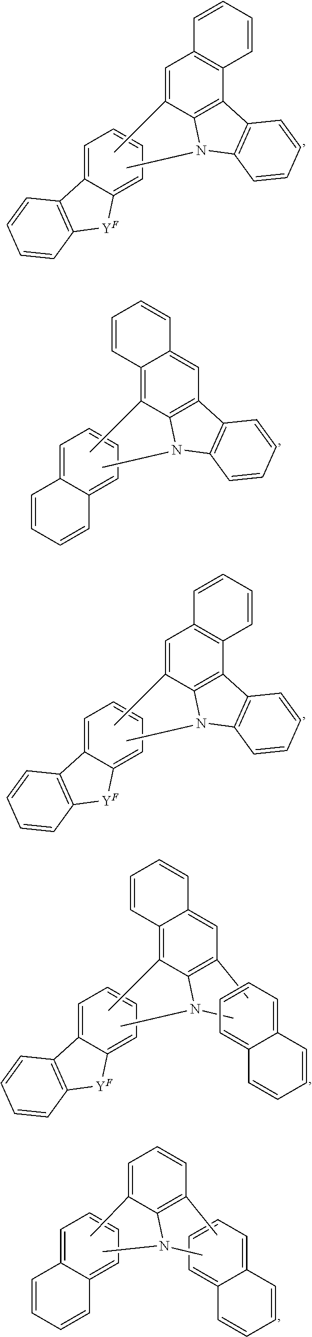

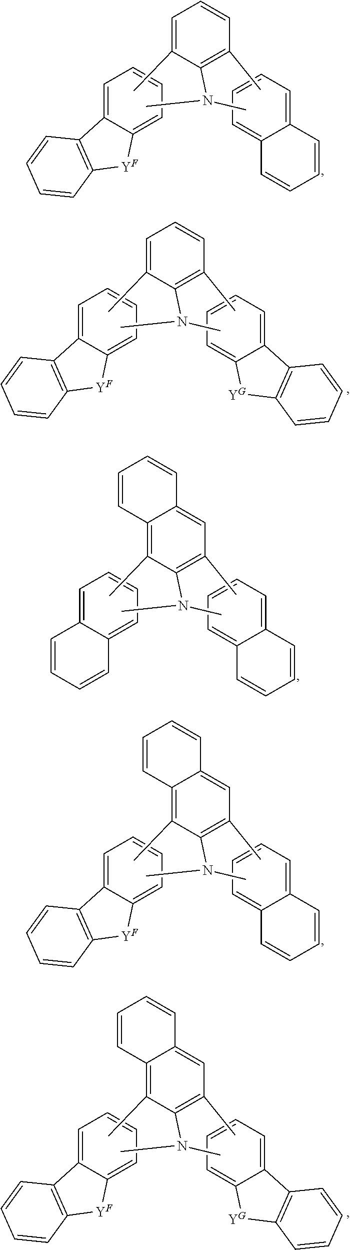

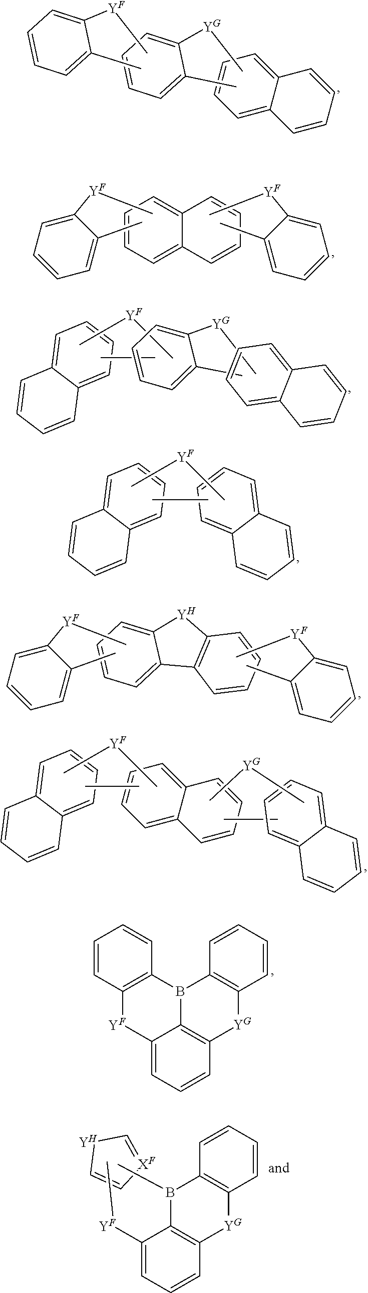

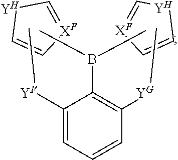

wherein Y^{F}, Y^{G}, Y^{H}, and Y^{I} are each independently selected from the group consisting of B, C, Si, Ge, N, P, O, S, Se, C═O, S═O, and SO₂;
wherein X^{F} and X^{G} are each independently selected from the group consisting of C and N.

In some of the above embodiments, any carbon ring atoms up to maximum of a total number of three, together with their substituents, in each phenyl ring of any of above structures can be replaced with N.

### f) HBL:

A hole blocking layer (HBL) may be used to reduce the number of holes and/or excitons that leave the emissive layer. The presence of such a blocking layer in a device may result in substantially higher efficiencies and/or longer lifetime as compared to a similar device lacking a blocking layer. Also, a blocking layer may be used to confine emission to a desired region of an OLED. In some embodiments, the HBL material has a lower HOMO (further away from the vacuum level) and/or higher triplet energy than one or more of the emitters closest to the HBL interface.

In some embodiments, the compound used in HBL contains the same molecule or the same functional groups used as host described above.

In some embodiments, compound used in HBL comprises at least one of the following moieties selected from the group consisting of:

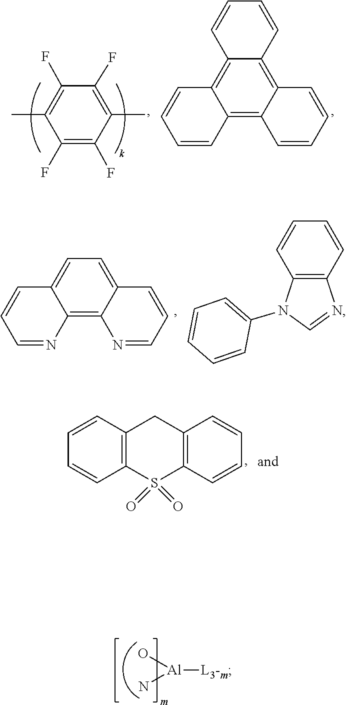

wherein k is an integer from 1 to 20; L¹⁰¹ is another ligand, k′ is an integer from 1 to 3.

### g) ETL:

Electron transport layer (ETL) may include a material capable of transporting electrons. Electron transport layer may be intrinsic (undoped), or doped. Doping may be used to enhance conductivity. Examples of the ETL material are not particularly limited, and any metal complexes or organic compounds may be used as long as they are typically used to transport electrons.

In some embodiments, compound used in ETL comprises at least one of the following moieties in the molecule:

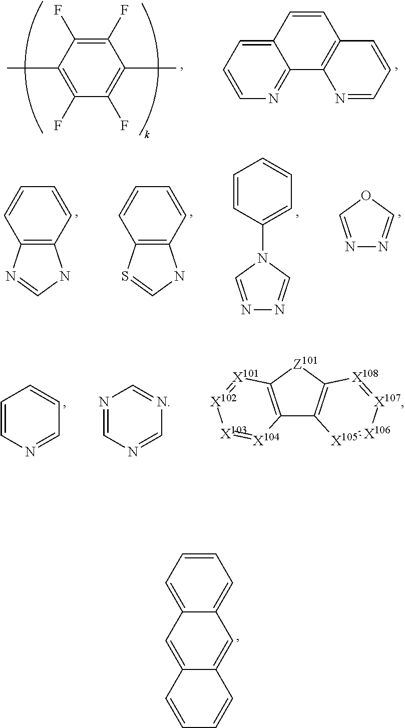

and fullerenes; wherein k is an integer from 1 to 20, X¹⁰¹ to X¹⁰⁸ is selected from C or N; Z¹⁰¹ is selected from the group consisting of C, N, O, and S.

In some embodiments, the metal complexes used in ETL contains, but not limit to the following general formula:

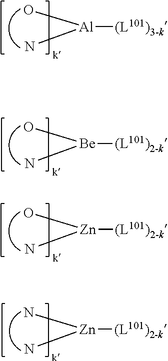

wherein (O—N) or (N—N) is a bidentate ligand, having metal coordinated to atoms O, N or N, N; L¹⁰¹ is another ligand; k′ is an integer value from 1 to the maximum number of ligands that may be attached to the metal.

In some embodiments, the ETL material is selected from the group consisting of anthracene-benzoimidazole compounds, aza triphenylene derivatives, anthracene-benzothiazole compounds, metal 8-hydroxyquinolates, metal hydroxybenoquinolates, bathocuprine compounds, 5-member ring electron deficient heterocycles (e.g., triazole, oxadiazole, imidazole, benzoimidazole), silole compounds, arylborane compounds, fluorinated aromatic compounds, fullerene (e.g., C60), triazine complexes, and Zn (N\hat{ }N) complexes.

### h) Charge Generation Layer (CGL)

In tandem or stacked OLEDs, the CGL plays an essential role in the performance, which is composed of an n-doped layer and a p-doped layer for injection of electrons and holes, respectively. Electrons and holes are supplied from the CGL and electrodes. The consumed electrons and holes in the CGL are refilled by the electrons and holes injected from the cathode and anode, respectively; then, the bipolar currents reach a steady state gradually. Typical CGL materials include n and p conductivity dopants used in the transport layers.

In any above-mentioned compounds used in each layer of the OLED device, the hydrogen atoms can be partially or fully deuterated. The minimum amount of hydrogen of the compound being deuterated is selected from the group consisting of 30%, 40%, 50%, 60%, 70%, 80%, 90%, 95%, 99%, and 100%. As used herein, percent deuteration has its ordinary meaning and includes the percent of all possible hydrogen and deuterium atoms that are replaced by deuterium atoms. In some embodiments, the deuterium atoms are attached to an aromatic ring. In some embodiments, the deuterium atoms are attached to a saturated carbon atom, such as an alkyl or cycloalkyl carbon atom. In some other embodiments, the deuterium atoms are attached to a heteroatom, such as Si, or Ge atom.

It is understood that the various embodiments described herein are by way of example only and are not intended to limit the scope of the invention. For example, many of the materials and structures described herein may be substituted with other materials and structures without deviating from the spirit of the invention. The present invention as claimed may therefore include variations from the particular examples and preferred embodiments described herein, as will be apparent to one of skill in the art. It is understood that various theories as to why the invention works are not intended to be limiting.

What is claimed is:

1. An organic light emitting device (OLED) comprising:
an anode;
an emissive region;
a cathode;
wherein the emissive region comprises:
a first compound, D1, which is a first metal complex; and
a second compound, D2, which is a second metal complex;
wherein D1 and D2 are different; and
wherein the emission from the OLED does not primarily originate from D2 or from an exciplex between D1 and D2;
wherein the emission from the OLED has an emission peak maximum between 300 to 2000 nm; and
wherein, if the emission from the OLED has a peak between 550 and 700 nm, then D2 is not a Be, Al, or Ir complex.

2. The OLED of claim 1, wherein D2 comprises a metal M2 selected from the group consisting of Ir, Pt, Pd, Au, Ag, Cu, Zn, Zr, Ru, Re, Co, Ti, Os, Rh, Be, Mn, Mg, Ni, Fe, Tl, Pb, Po, At, Sb, Te, Al, Ga, In, Bi, and Sn; and/or
wherein D1 comprises a metal M1 selected from the group consisting of Ir, Os, Rh, Pt, Pd, Au, Ag, and Cu; and/or
wherein M1 and M2 are different.

3. The OLED of claim 1, wherein D1 is an Ir or Pt complex; and/or wherein D2 is a Pt or Pd complex; and/or wherein D1 has at least one ligand with the same structure as a ligand in D2.

4. The OLED of claim 1, wherein the emissive region comprises more than one layer; and/or
wherein D1 and D2 are in the same layer of the emissive region.

5. The OLED of claim 1, wherein D2 is a host material.

6. The OLED of claim 5, wherein D2 has the formula of M(L¹)ₓ(L²)_{y}(L³)_{z};
wherein L¹, L², and L³ can be the same or different;
wherein x is 1, 2, or 3;
wherein y is 0, 1, or 2;
wherein z is 0, 1, or 2;
wherein x+y+z is the oxidation state of the metal M;
wherein L¹ is selected from the group consisting of the structures of the following LIST 1: 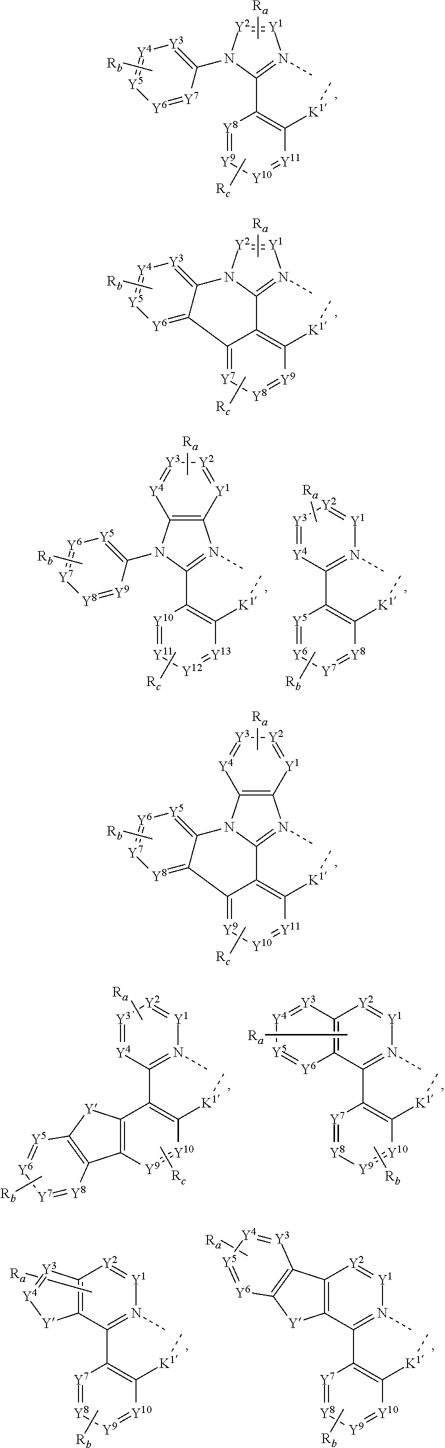 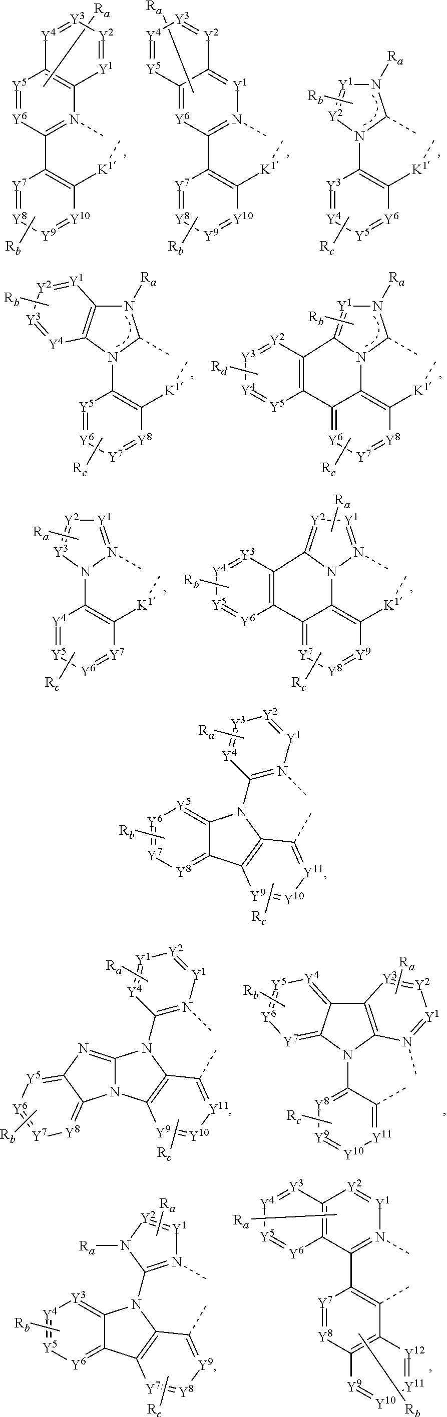 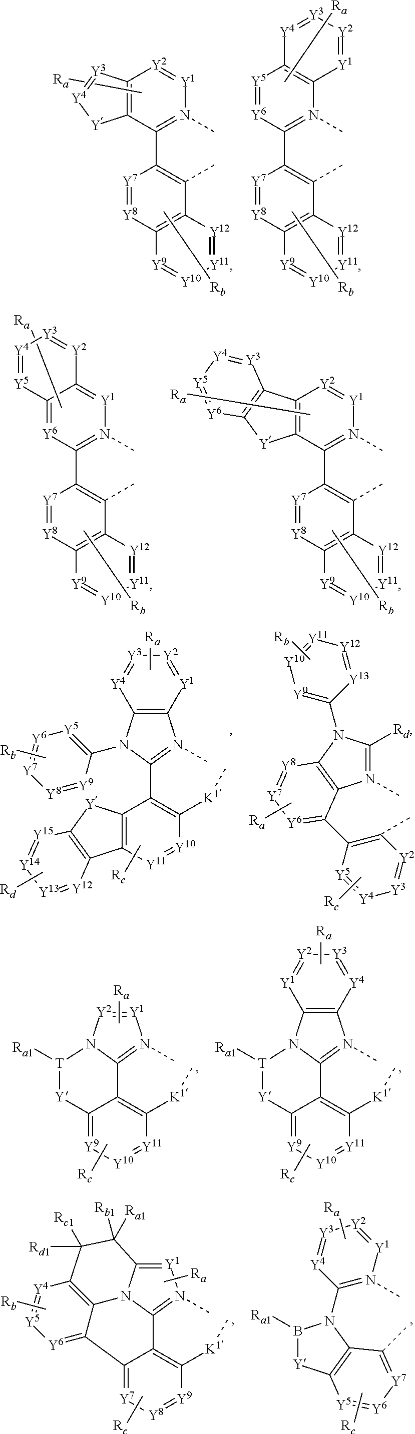 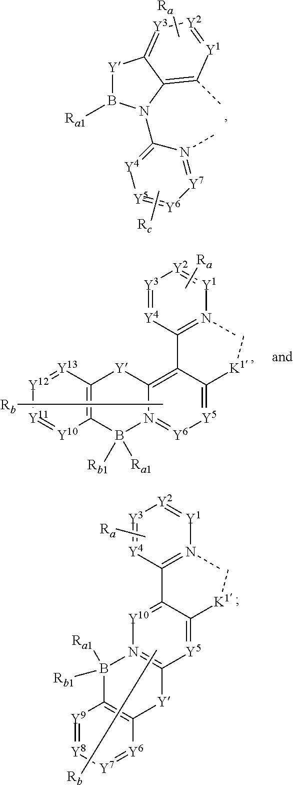
wherein L² and L³ are independently selected from the group consisting of 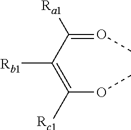
and the structures of the LIST 1;
wherein:
T is selected from the group consisting of B, Al, Ga, and In;
K^{1′} is a direct bond or is selected from the group consisting of NRₑ, PRₑ, O, S, and Se;
each Y¹ to Y¹³ are independently selected from the group consisting of carbon and nitrogen;
Y′ is selected from the group consisting of BRₑ, NRₑ, P Rₑ, O, S, Se, C═O, S═O, SO₂, CRₑR_{f}, SiRₑR_{f}, and GeRₑR_{f};
Rₑ and R_{f} can be fused or joined to form a ring;
each Rₐ, R_{b}, R_{c}, and R_{d} can independently represent from mono to the maximum possible number of substitutions, or no substitution;
each Rₐ₁, R_{b1}, R_{c1}, R_{d1}, Rₐ, R_{b}, R_{c}, R_{d}, Rₑ, and R_{f} is independently a hydrogen or a substituent selected from the group consisting of deuterium, halogen, alkyl, cycloalkyl, heteroalkyl, heterocycloalkyl, boryl, arylalkyl, alkoxy, aryloxy, amino, silyl, germyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof; and
any two adjacent substituents of Rₐ₁, R_{b1}, R_{c1}, R_{d1}, Rₐ, R_{b}, R_{c}, and R_{d} can be fused or joined to form a ring or form a multidentate ligand.

7. The OLED of claim 5, wherein D2 has a structure of Formula I: 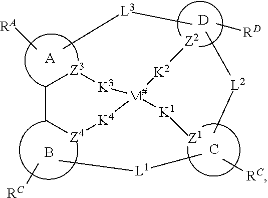
wherein:
M^{#} is Pd or Pt;
moieties A, B, C, and D are each independently monocyclic or polycyclic ring structure comprising 5-membered and/or 6-membered carbocyclic or heterocyclic rings;
Z¹, Z², Z³, and Z⁴ are each independently C or N;
K¹, K², K³, and K⁴ are each independently selected from the group consisting of a direct bond, O, and S, wherein at least two of K¹, K², K³, and K⁴ are direct bonds;
L¹, L², and L³ are each independently selected from the group consisting of absent a bond, a direct bond, BR, BRR′, NR, PR, P(O)R, O, S, Se, C═O, C═S, C═Se, C═NR, C═CRR′, S═O, SO₂, CR, CRR′, SiRR′, GeRR′, alkylene, cycloalkyl, aryl, cycloalkylene, arylene, heteroarylene, and combinations thereof, wherein at least one of L¹ and L² is present;
R^{A}, R^{B}, R^{C} and R^{D} each independently represents zero, mono, or up to a maximum allowed number of substitutions to its associated ring;
each of R, R′, R^{A}, R^{B}, R^{C}, and R^{D} is independently a hydrogen or a substituent selected from the group consisting of deuterium, fluorine, alkyl, cycloalkyl, heteroalkyl, alkoxy, aryloxy, amino, silyl, boryl, alkenyl, cycloalkenyl, heteroalkenyl, aryl, heteroaryl, nitrile, isonitrile, sulfanyl, and combinations thereof; and
any two R, R′, R^{A}, R^{B}, R^{C}, R^{E}, and R^{F} can be joined or fused together to form a ring.

8. The OLED of claim 5, wherein D2 has a structure selected from the group consisting of: 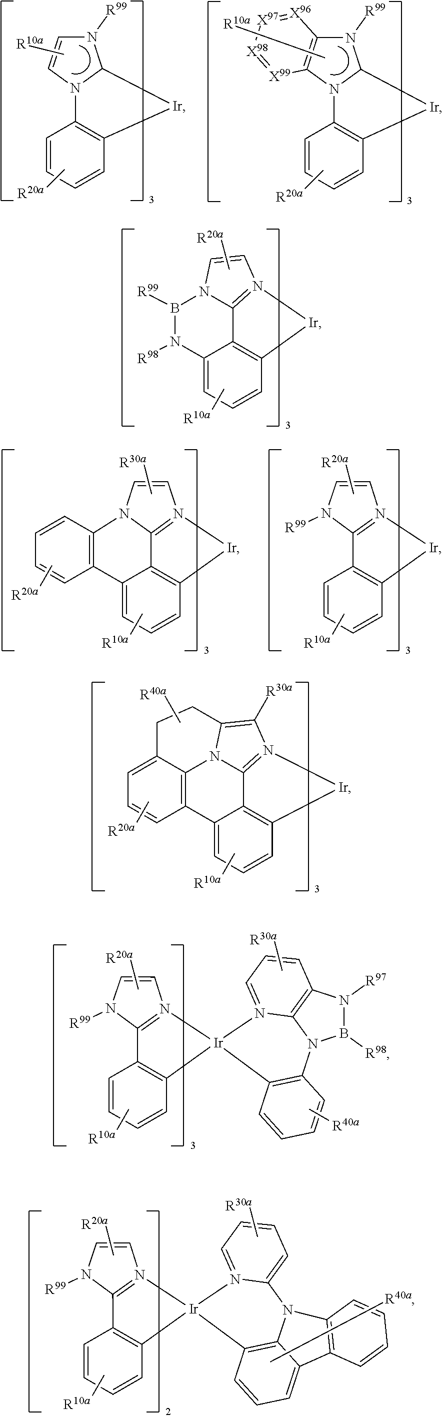 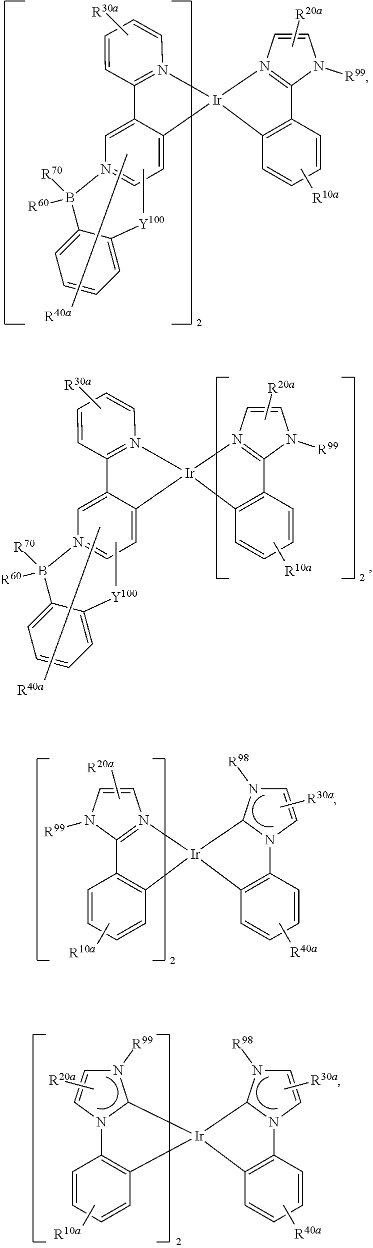 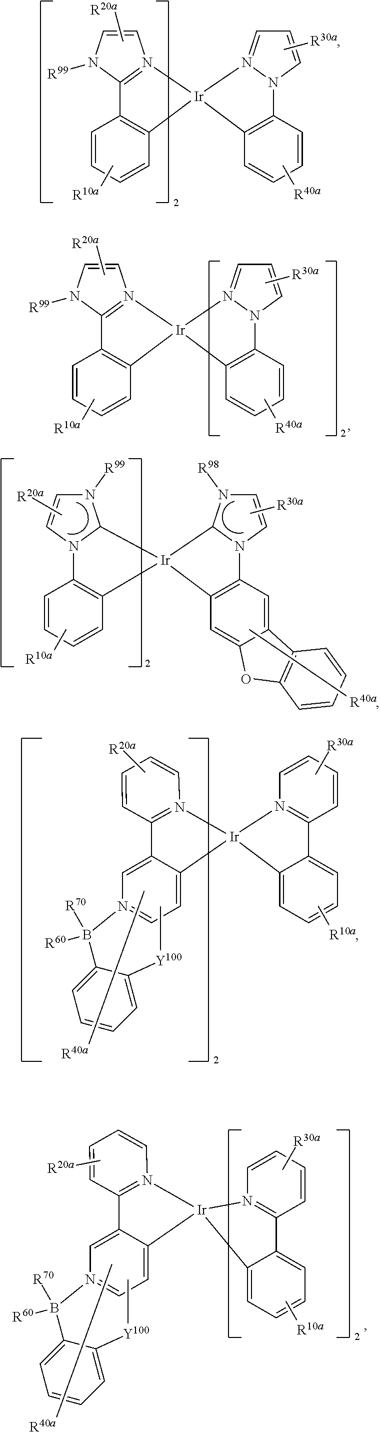 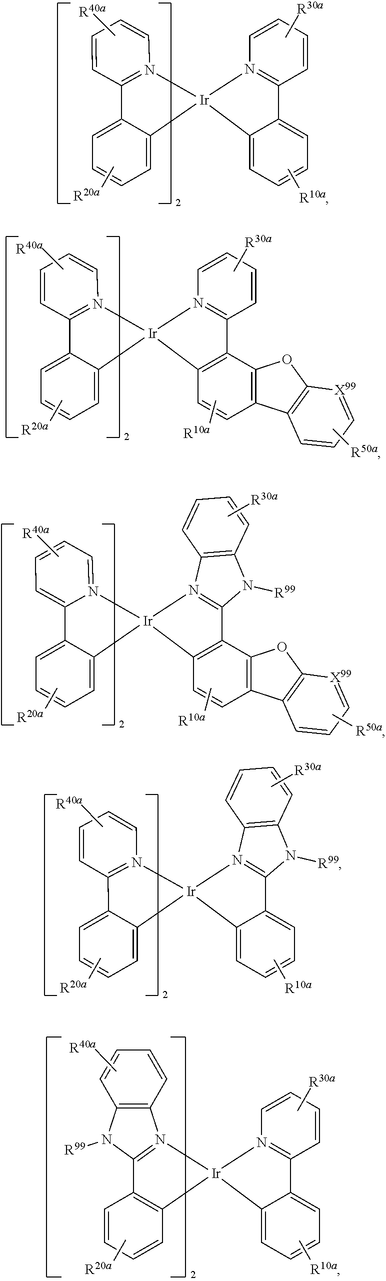 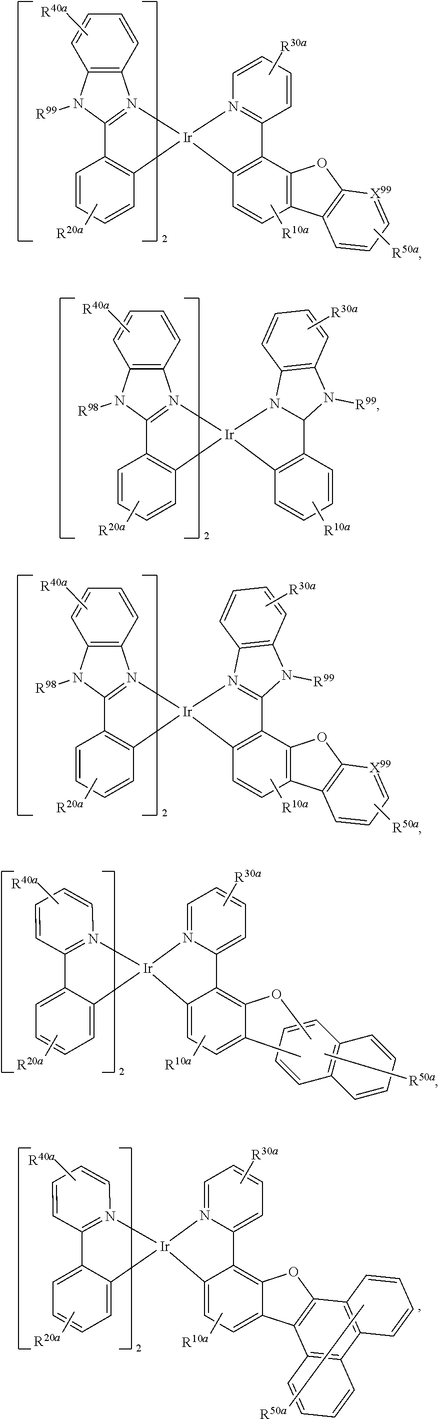 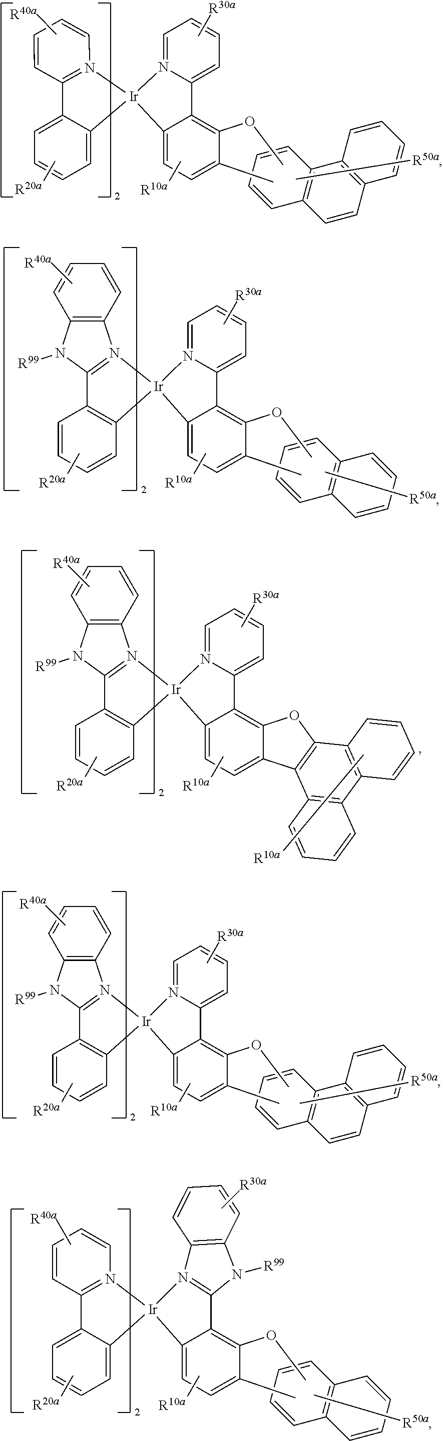 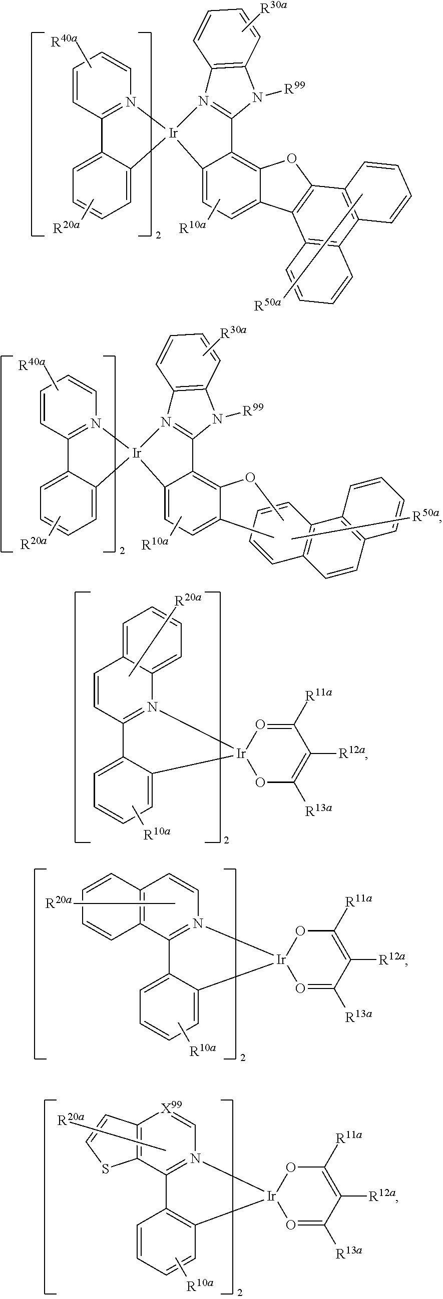 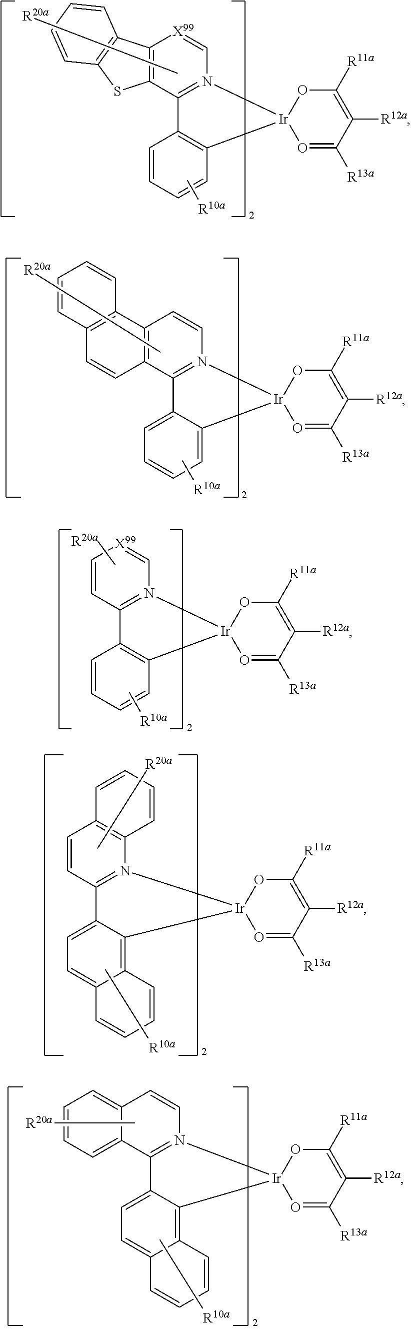 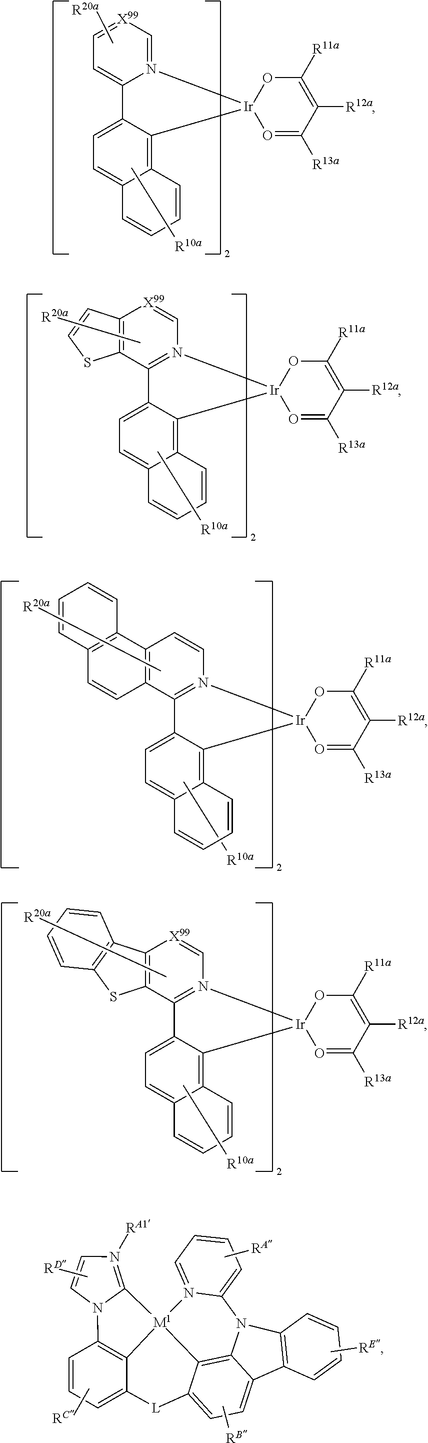 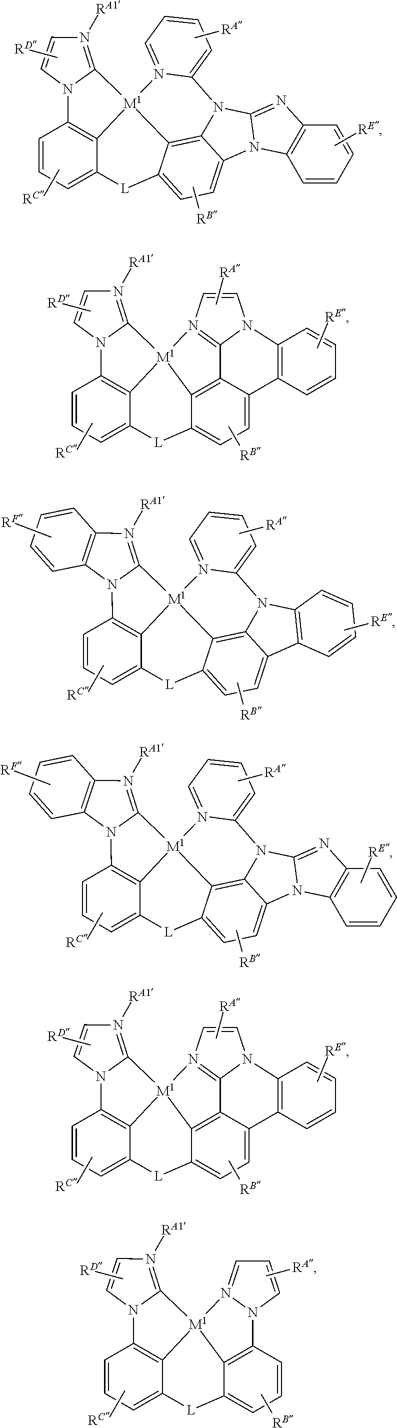 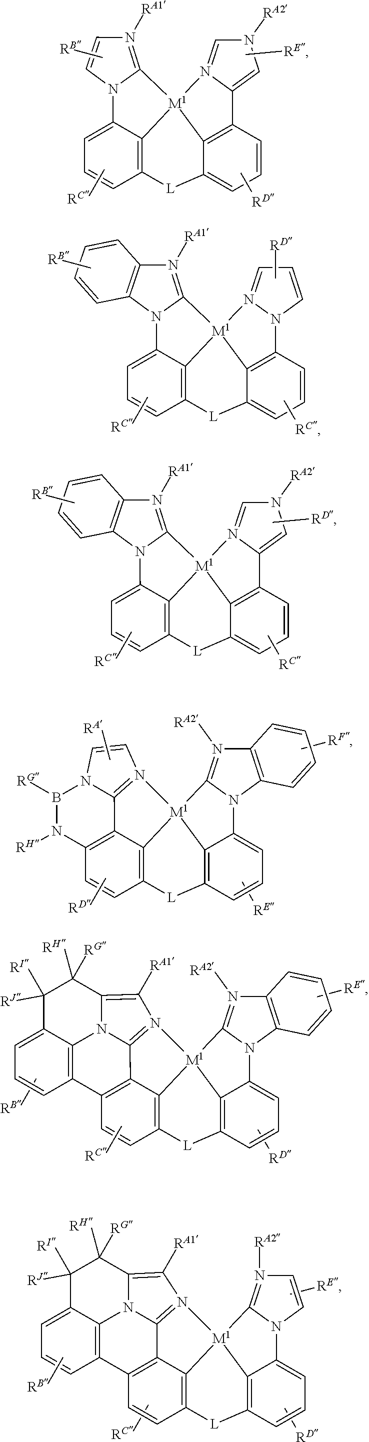 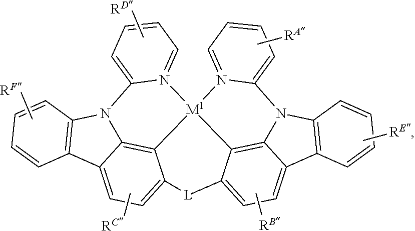 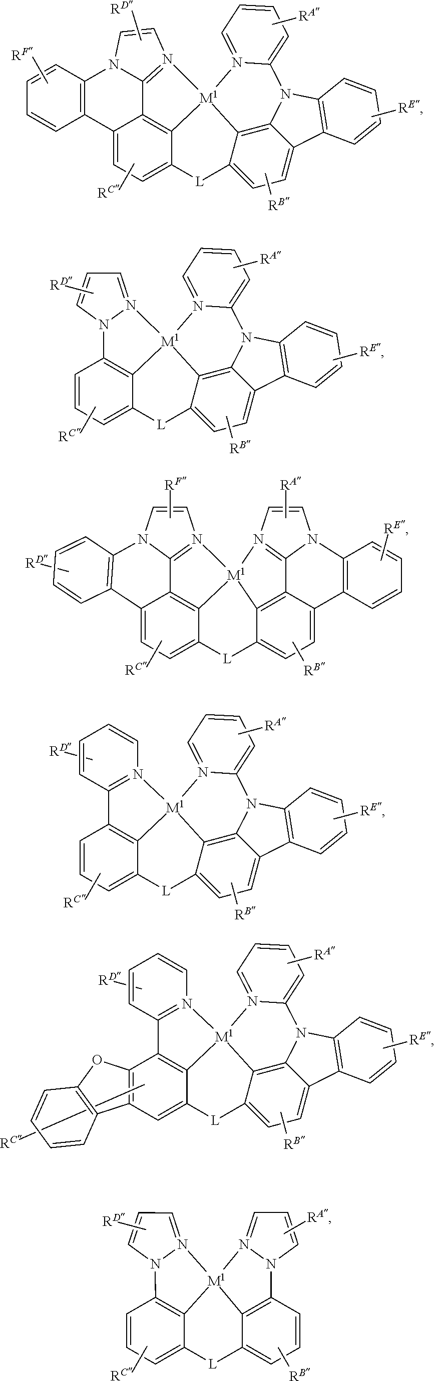 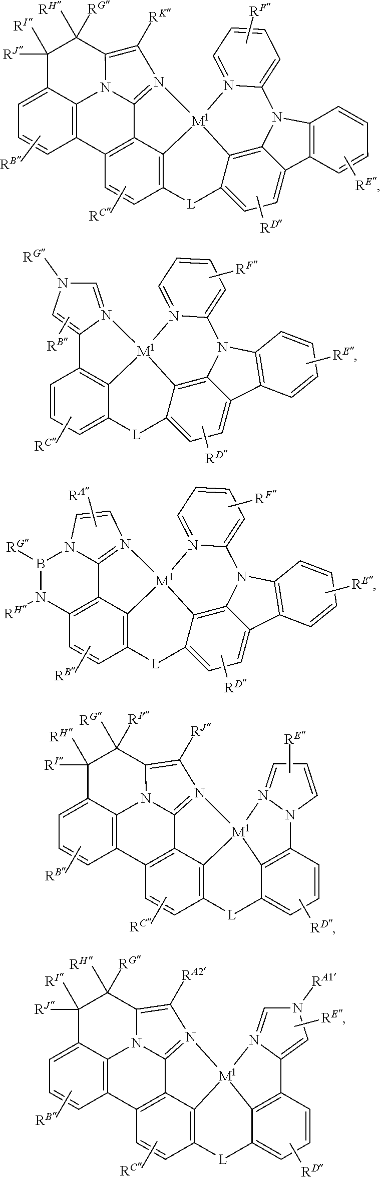 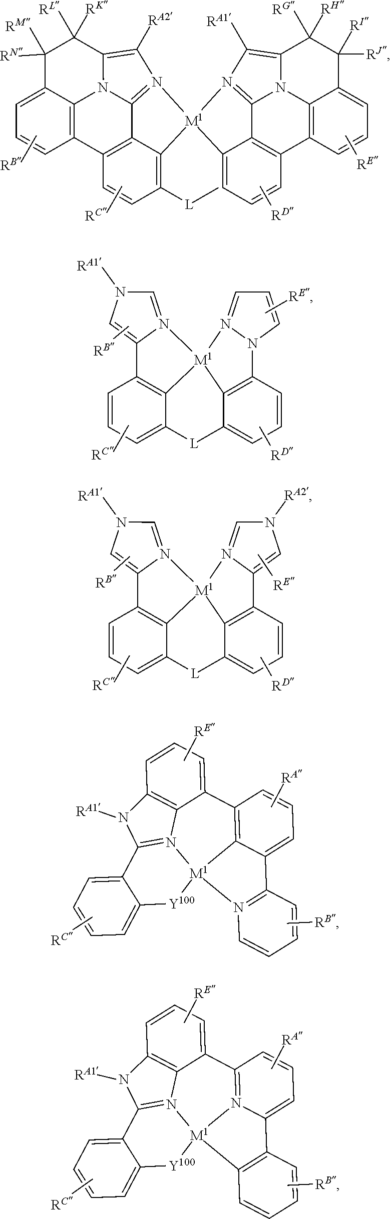 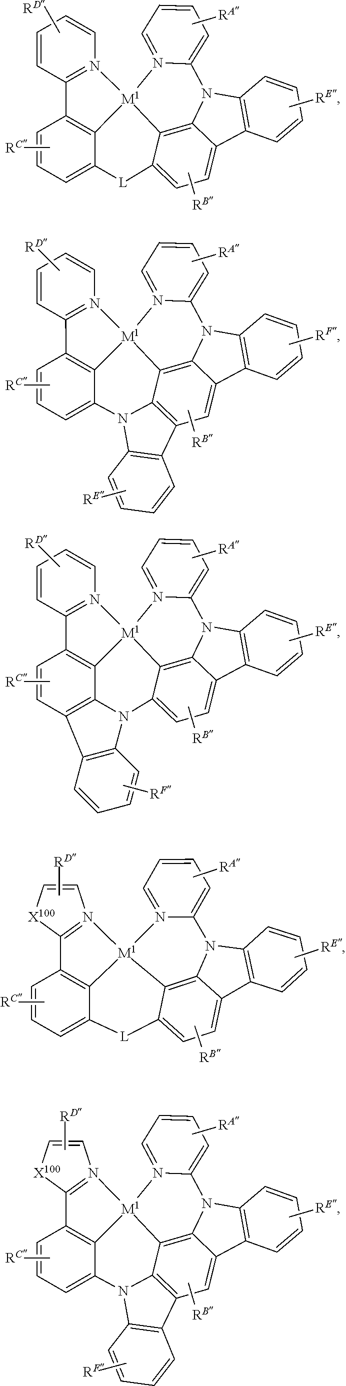 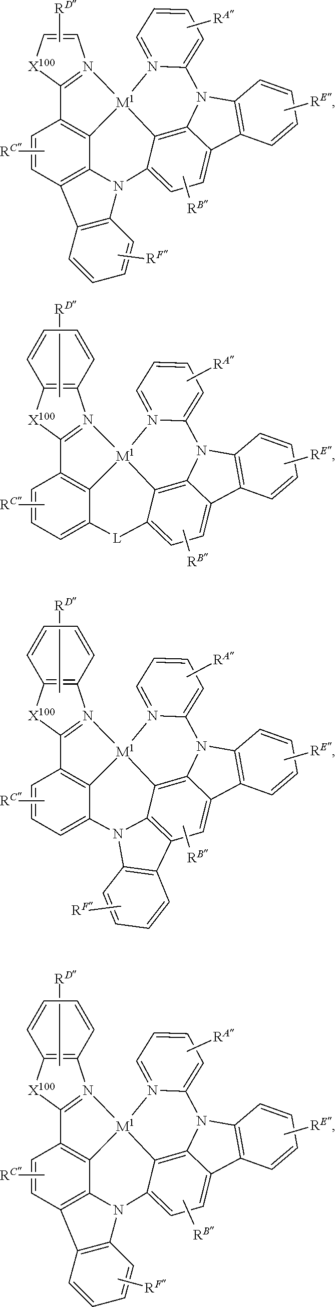 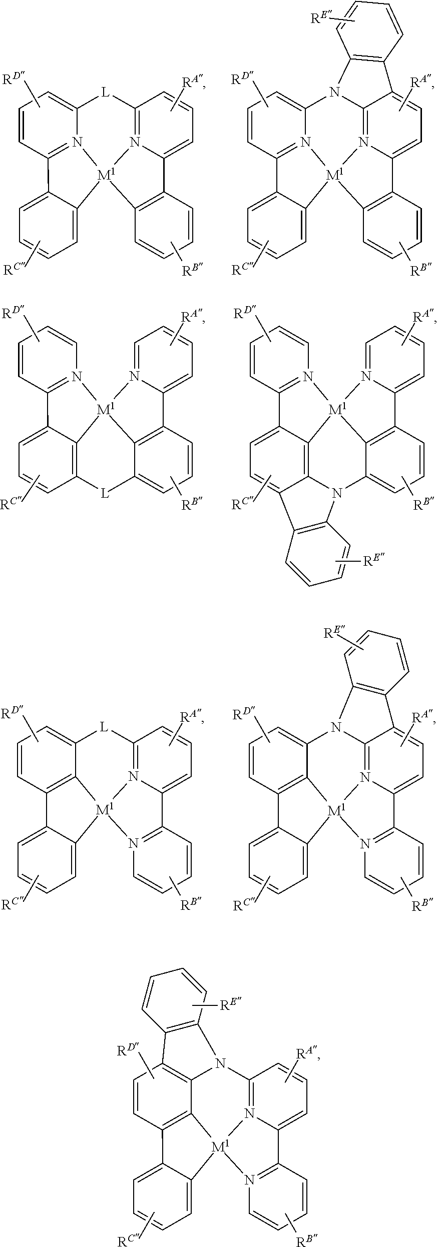 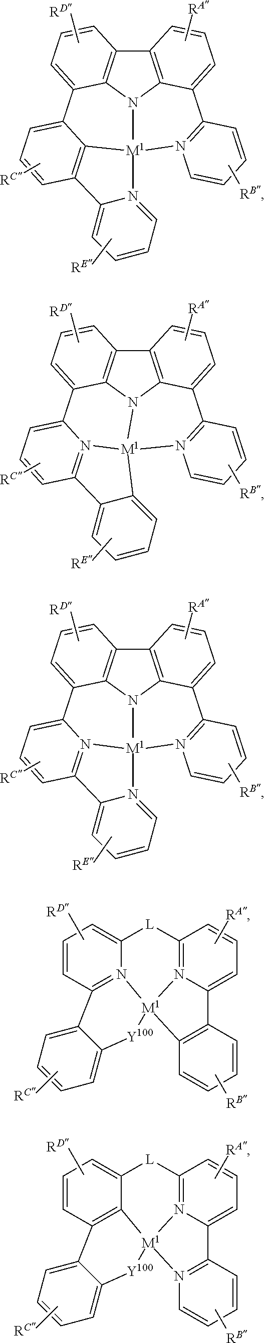 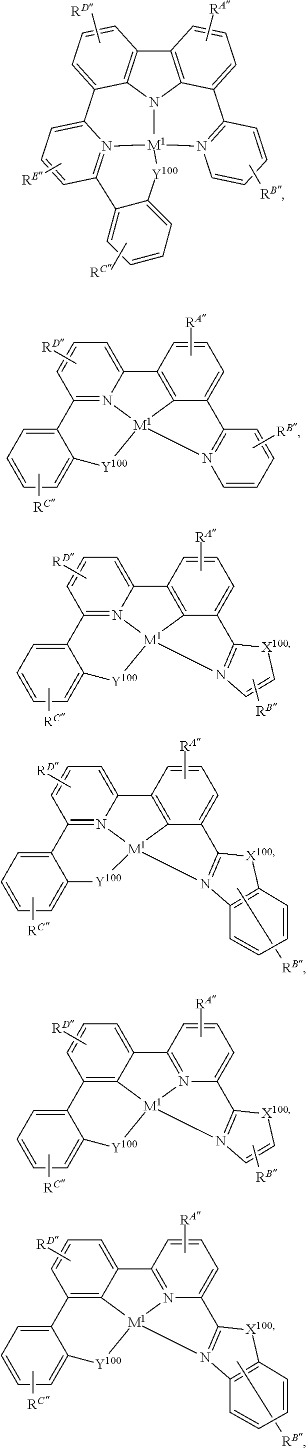 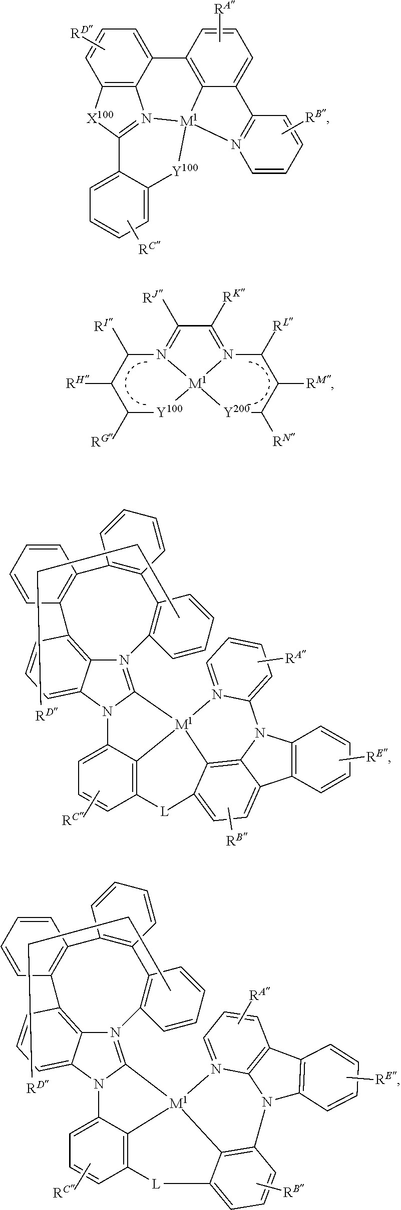 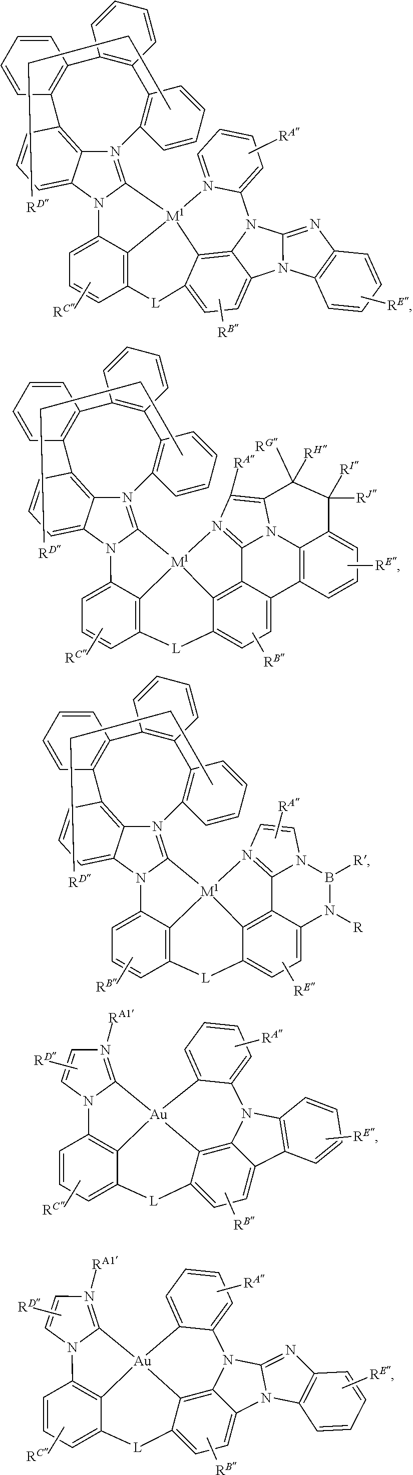 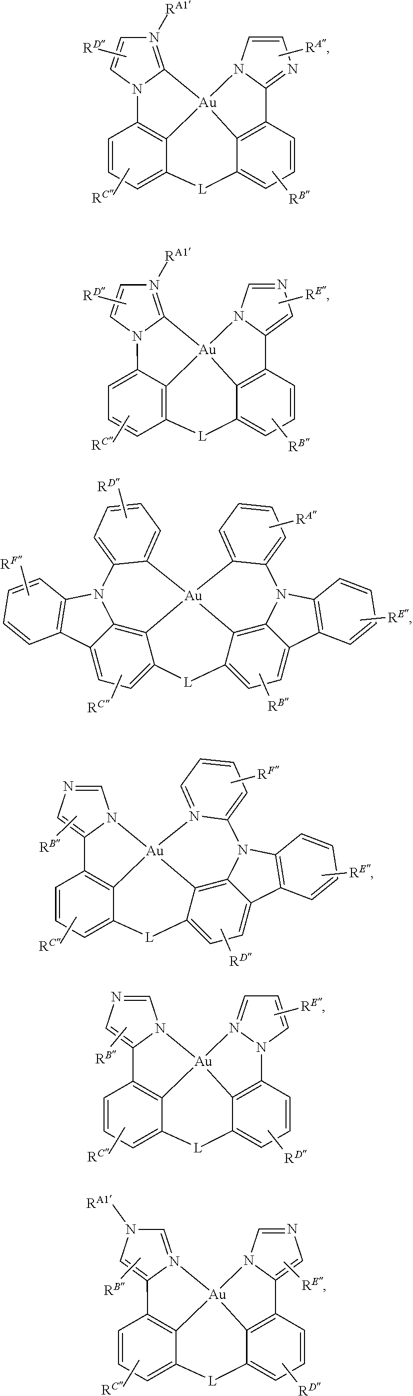 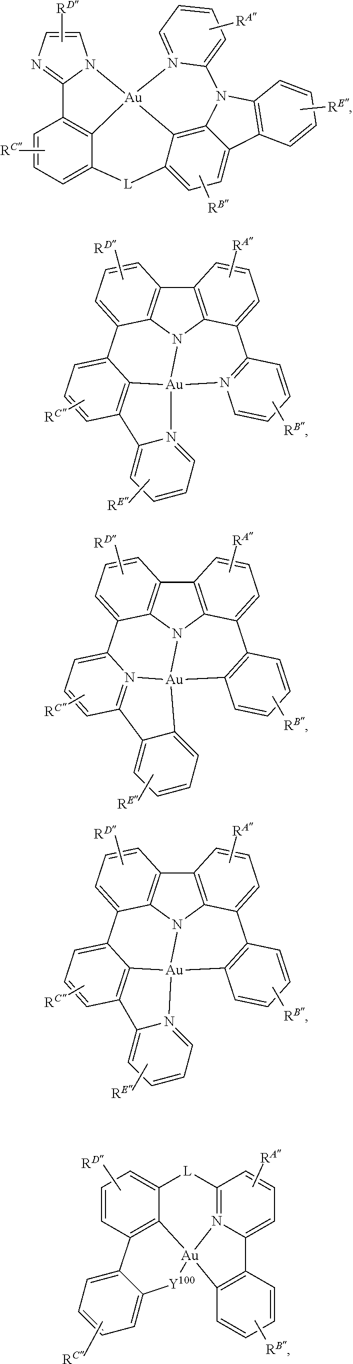 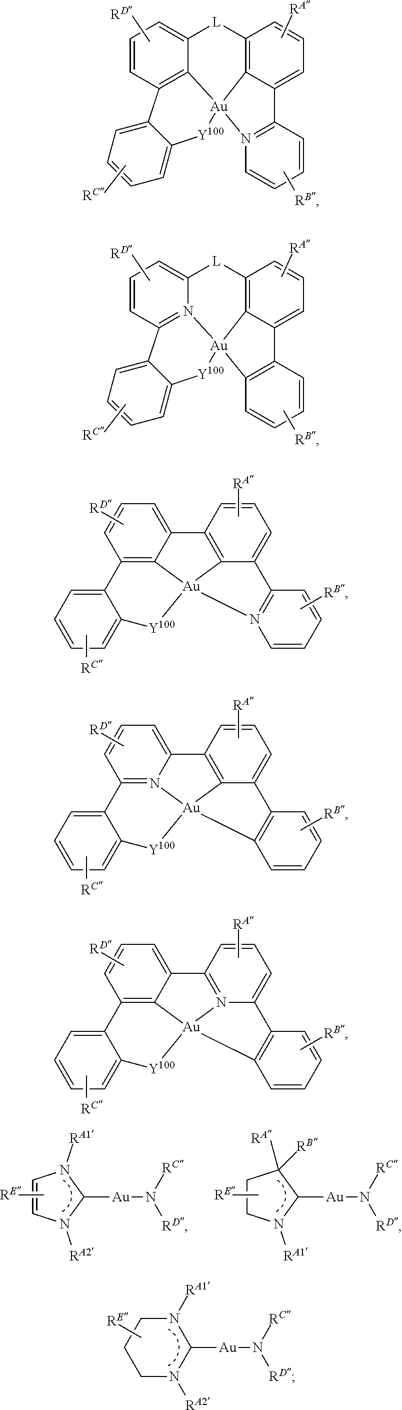
wherein:
M1 is selected from the group consisting of Pt, Pd, and Ni;
each of X⁹⁶ to X⁹⁹ is independently C or N;
each of Y¹⁰⁰ and Y²⁰⁰ is independently selected from the group consisting of a NR″, O, S, and Se;
L is independently selected from the group consisting of a direct bond, BR″, BR″R′″, NR″, PR″, O, S, Se, C═O, C═S, C═Se, C═NR″, C═CR″R′″, S═O, SO₂, CR″, CR″R′″, SiR″R′″, GeR″R′″, alkyl, cycloalkyl, aryl, heteroaryl, and combinations thereof;
X¹⁰⁰ for each occurrence is selected from the group consisting of O, S, Se, NR″, and CR″R′″;
each R^{10a}, R^{20a}, R^{30a}, R^{40a}, R^{50a}, R^{A″}, R^{B″}, R^{C″}, R^{D″}, R^{E″}, and R^{F}″ independently represents mono-, up to the maximum substitutions, or no substitutions;
each R, R′, R″, R′″, R^{10a}, R^{11a}, R^{12a}, R^{13a}, R^{20a}, R^{30a}, R^{40a}, R^{50a}, R⁶⁰, R⁷⁰, R⁹⁷, R⁹⁸, R⁹⁹, R^{A1′}, R^{A2′}, R^{A″}, R^{B″}, R^{C″}, R^{D″}, R^{E″}, R^{F″}, R^{G″}, R^{H″}, R^{I″}, R^{J″}, R^{K″}, R^{L″}, R^{M″}, and R^{N}″ is independently a hydrogen or a substituent selected from the group consisting of deuterium, halogen, alkyl, cycloalkyl, heteroalkyl, heterocycloalkyl, boryl, arylalkyl, alkoxy, aryloxy, amino, silyl, germyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof; and
any two adjacent R, R′, R″, R′″, R^{10a}, R^{11a}, R^{12a}, R^{13a}, R^{20a}, R^{30a}, R^{40a}, R^{50a}, R⁶⁰, R⁷⁰, R⁹⁷, R⁹⁸, R⁹⁹, R^{A1′}, R^{A2′}, R^{A″}, R^{B″}, R^{C″}, R^{D″}, R^{E″}, R^{F″}, R^{G″}, R^{H″}, R^{I″}, R^{J″}, R^{K″}, R^{L″}, R^{M″}, and R^{N}″ are optionally joined or fused to form a ring.

9. The OLED of claim 1, wherein the compound D2 has a structure selected from the group consisting of:

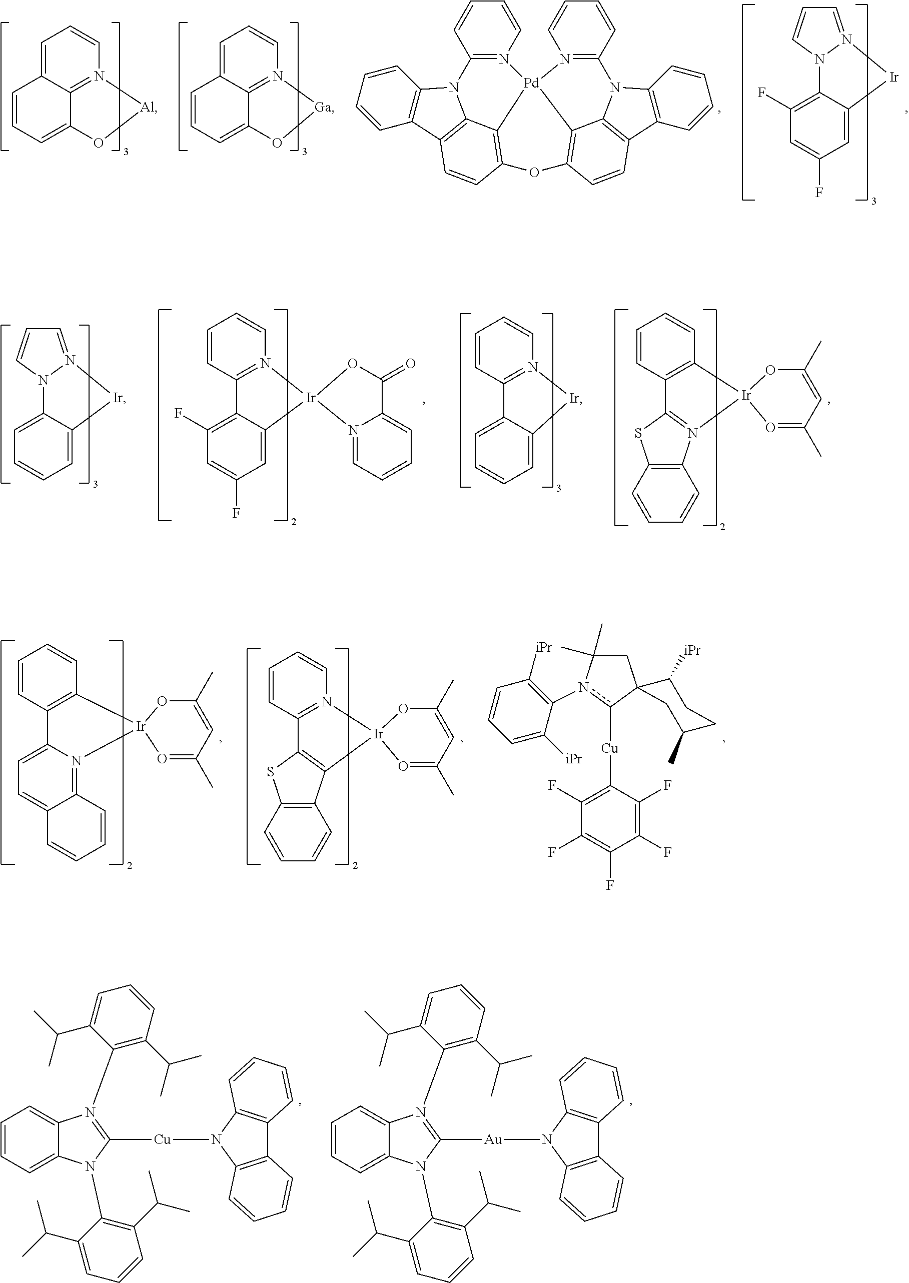

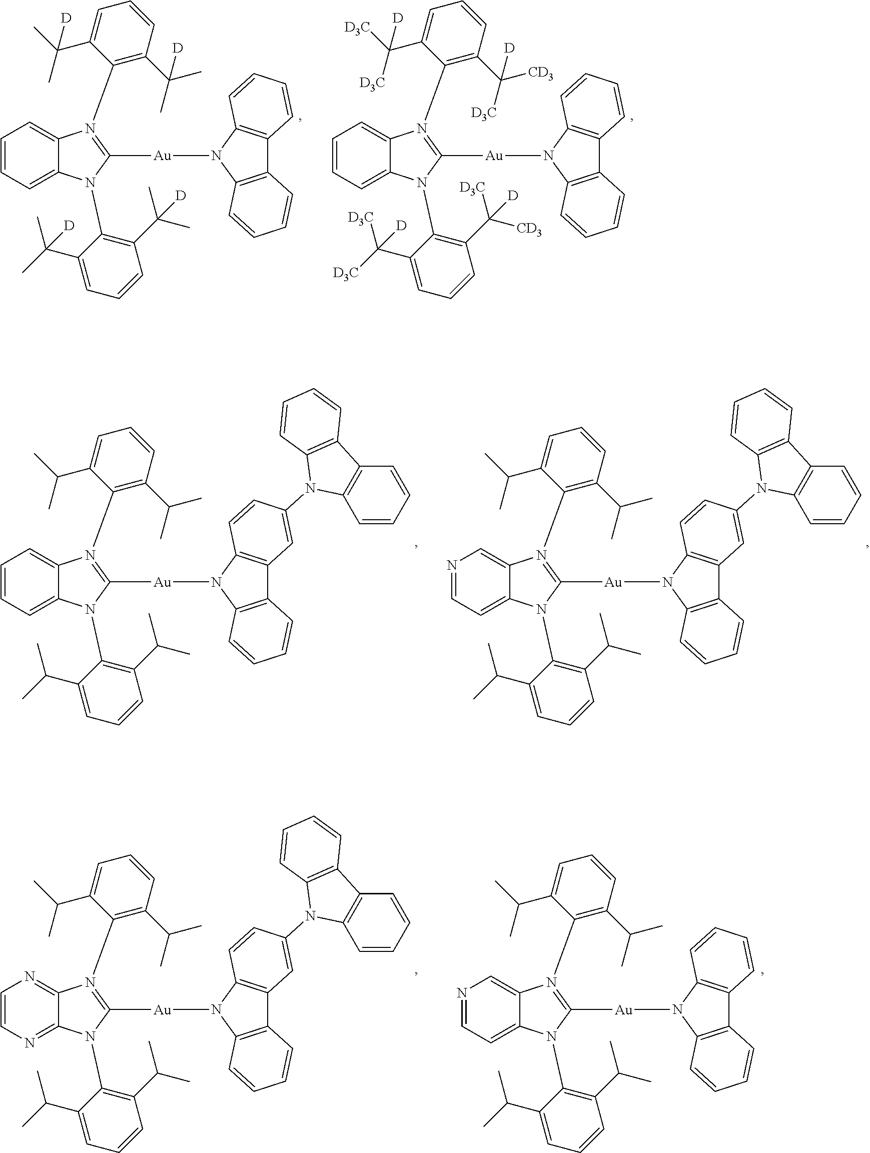

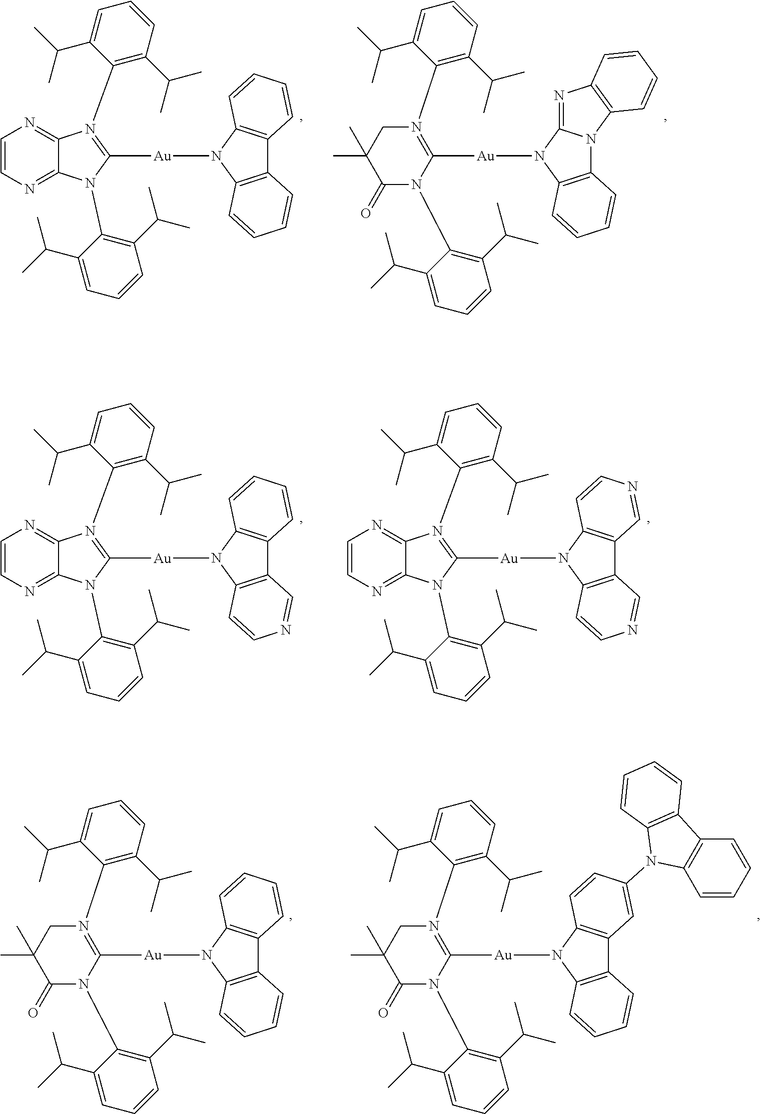

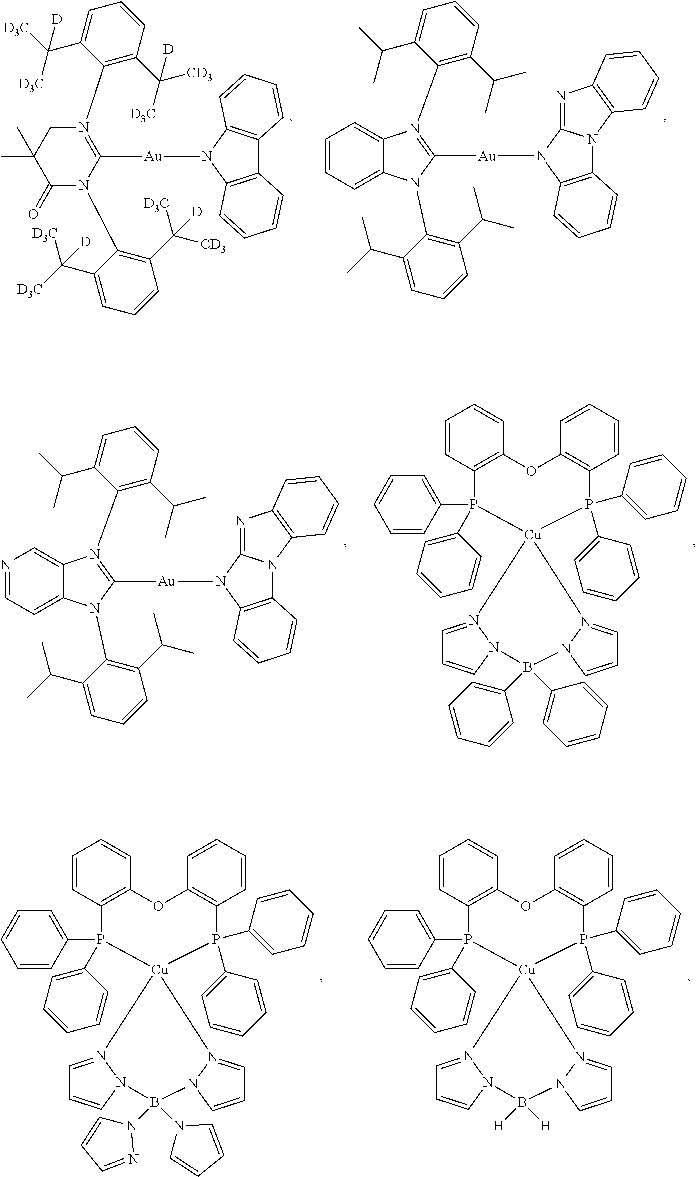

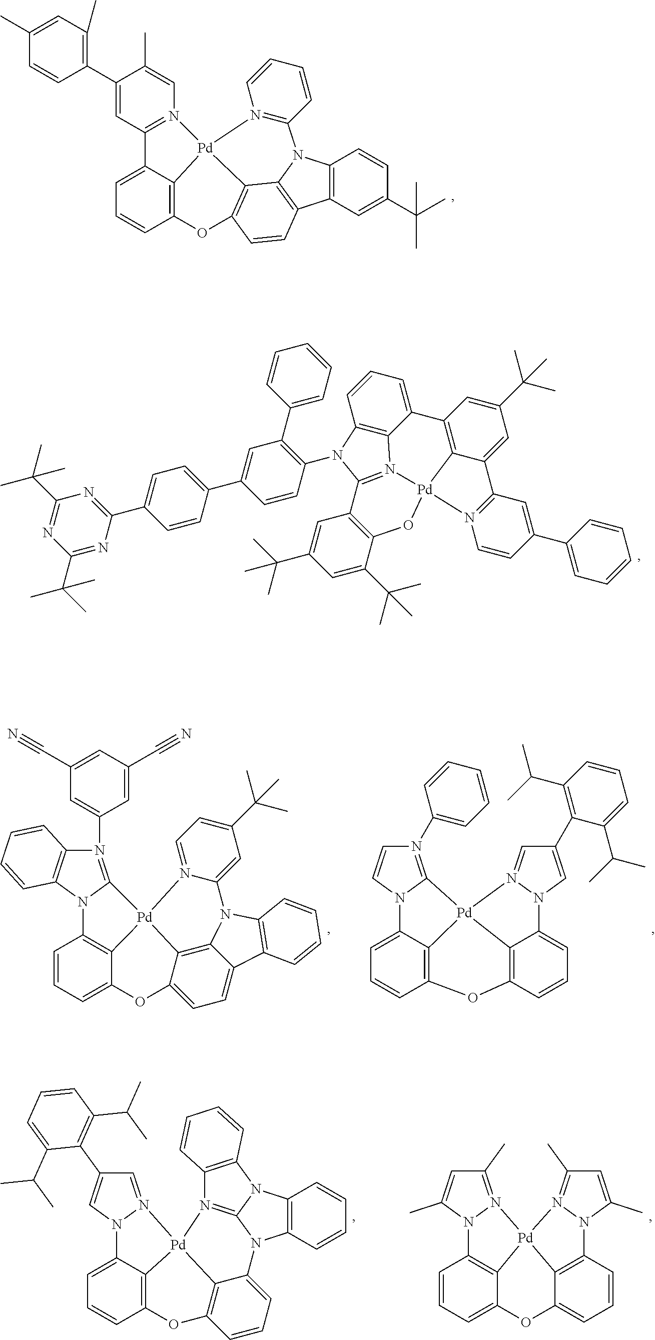

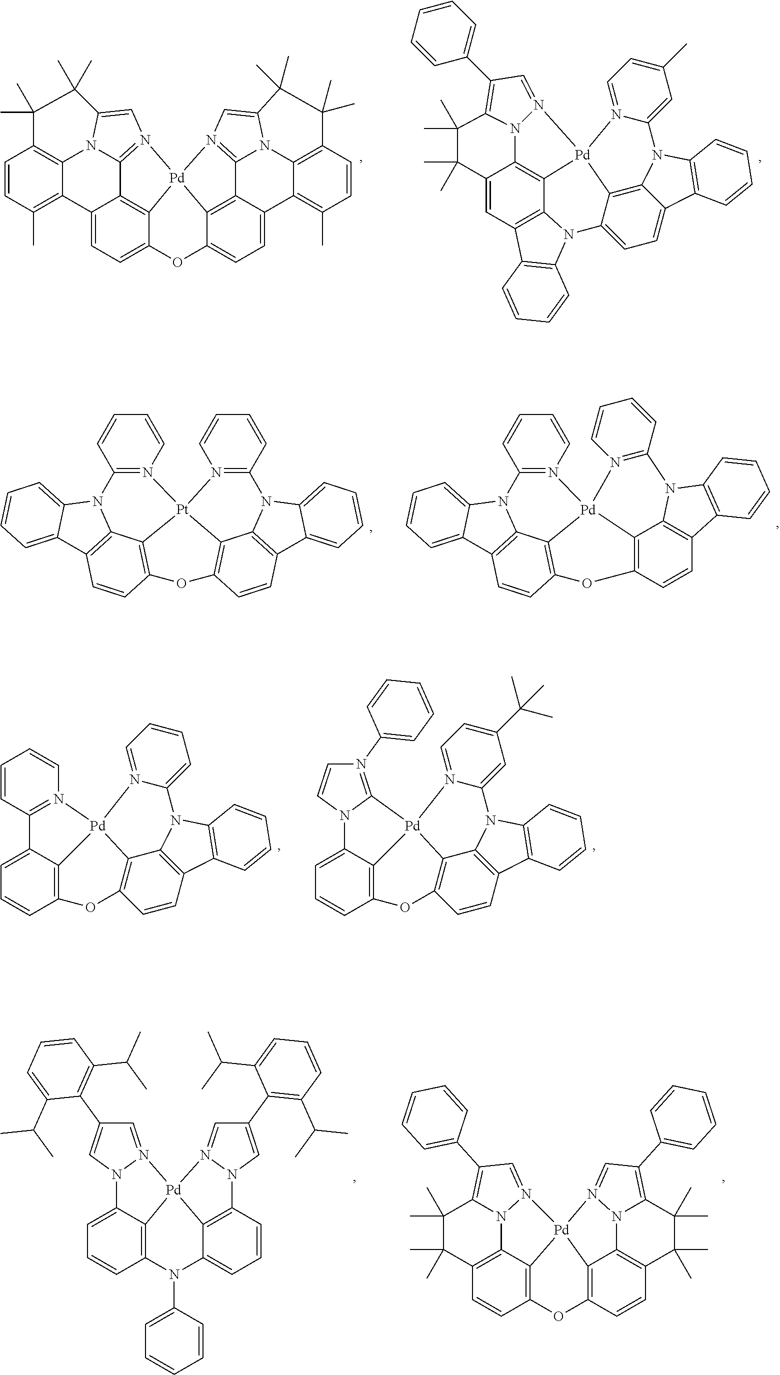

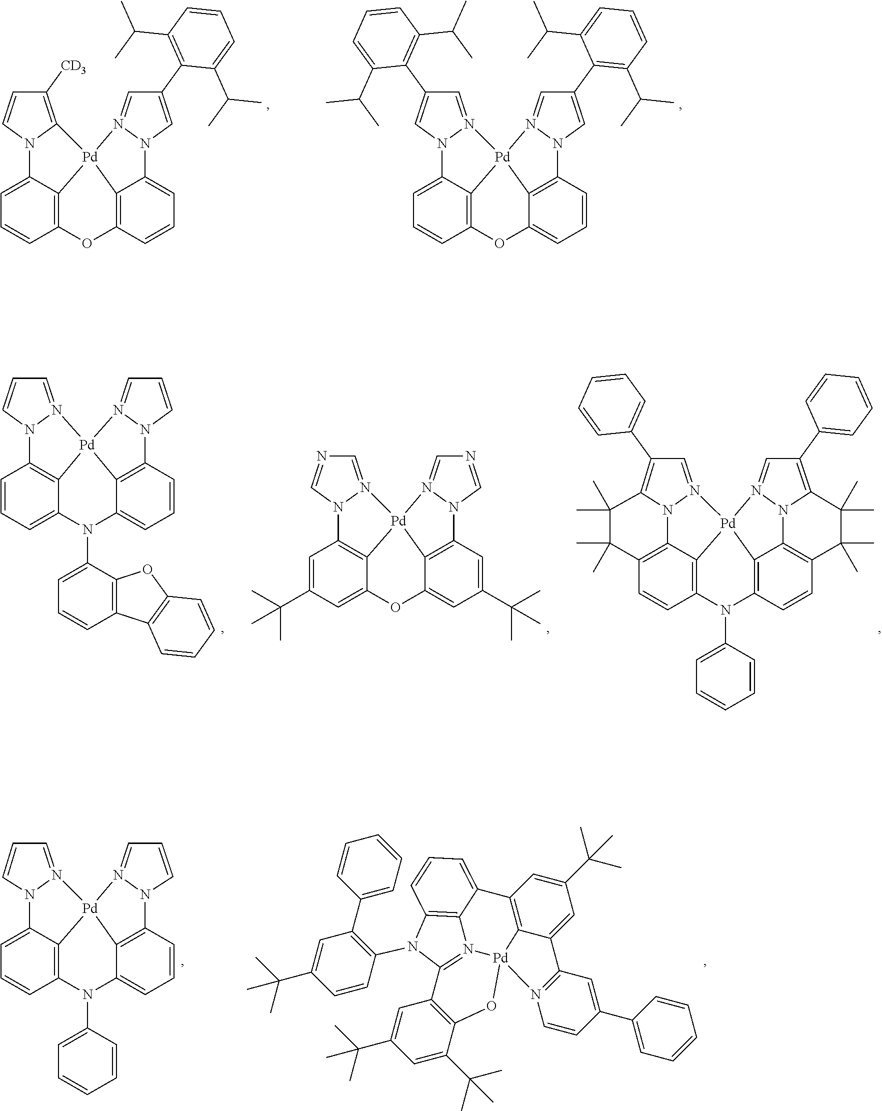

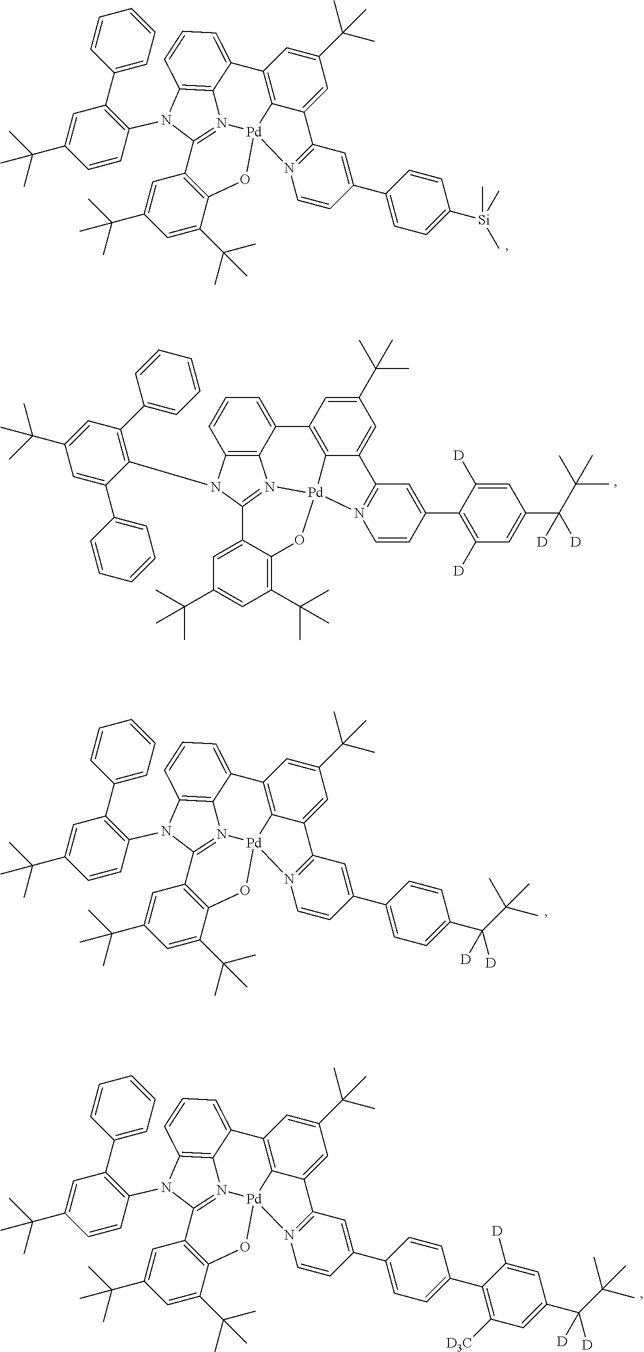

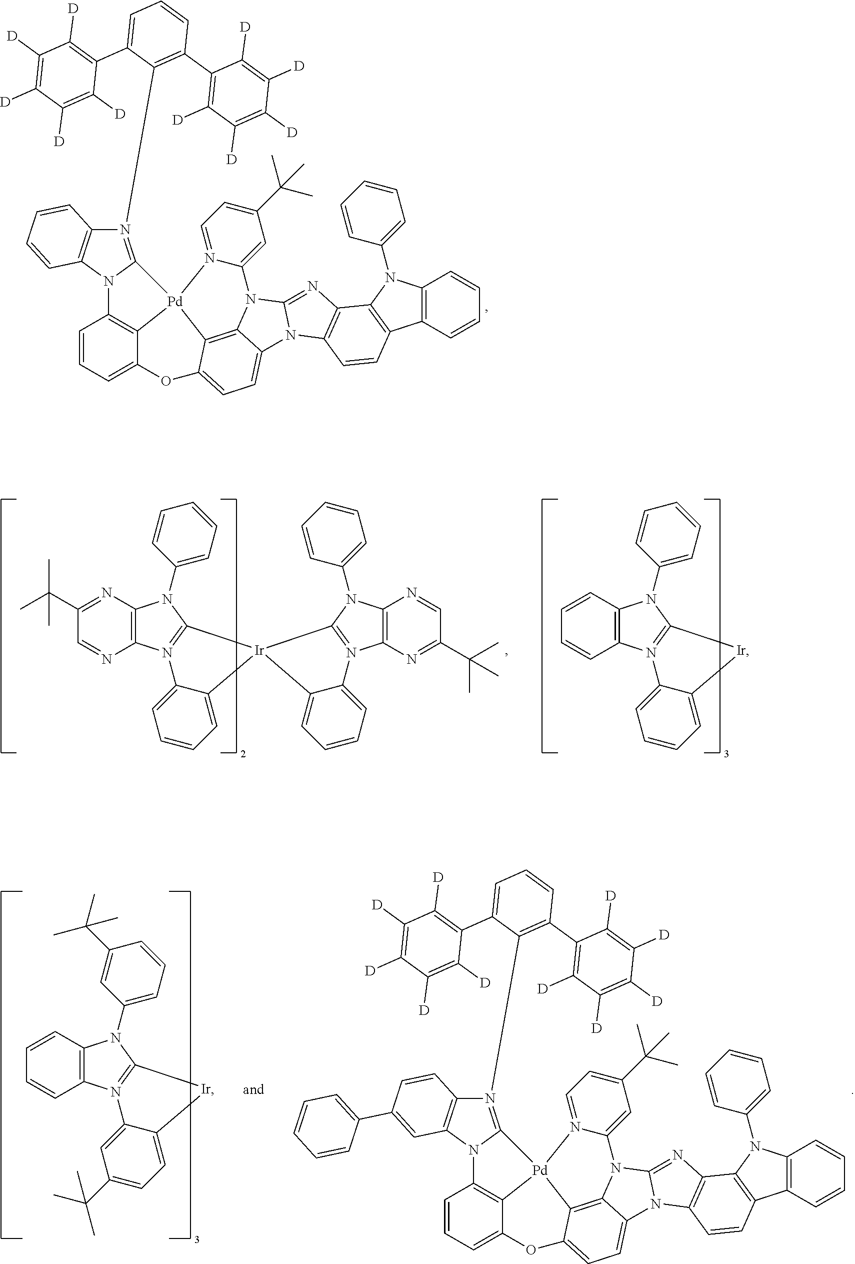

10. The OLED of claim 1, wherein the OLED further comprises a first host compound H1.

11. The OLED of claim 10, wherein the emissive region further comprises the first host compound H1.

12. The OLED of claim 10, wherein the first host compound H1 comprises at least one chemical moiety selected from the group consisting of triphenylene, carbazole, indolocarbazole, dibenzothiophene, dibenzofuran, dibenzoselenophene, 5λ²-benzo[d]benzo[4,5]imidazo[3,2-a]imidazole, 5,9-dioxa-13b-boranaphtho[3,2,1-de]anthracene, triazine, boryl, silyl, aza-triphenylene, aza-carbazole, aza-indolocarbazole, aza-dibenzothiophene, aza-dibenzofuran, aza-dibenzoselenophene, aza-5λ²-benzo[d]benzo[4,5]imidazo[3,2-a]imidazole, and aza-(5,9-dioxa-13b-boranaphtho[3,2,1-de]anthracene).

13. The OLED of claim 10, wherein the first host compound H1 is selected from the group consisting of: 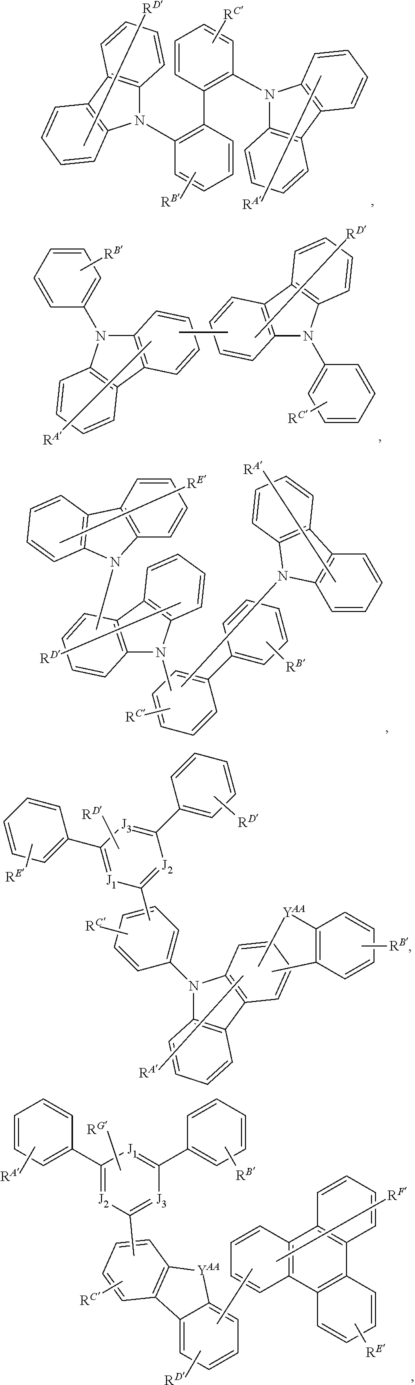 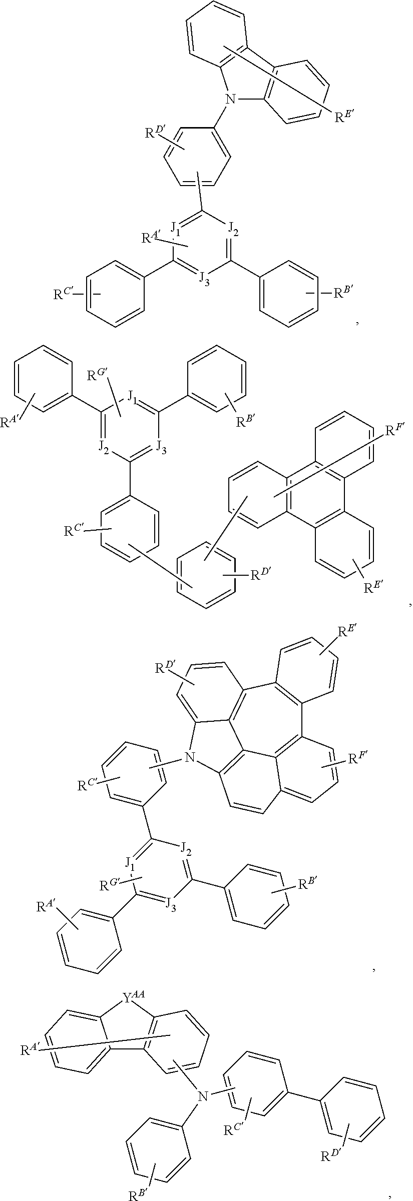 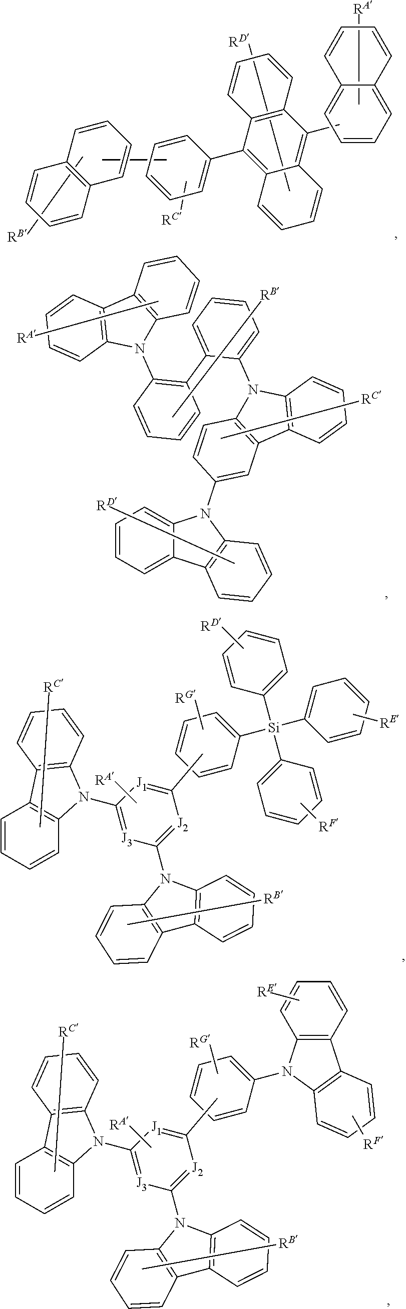 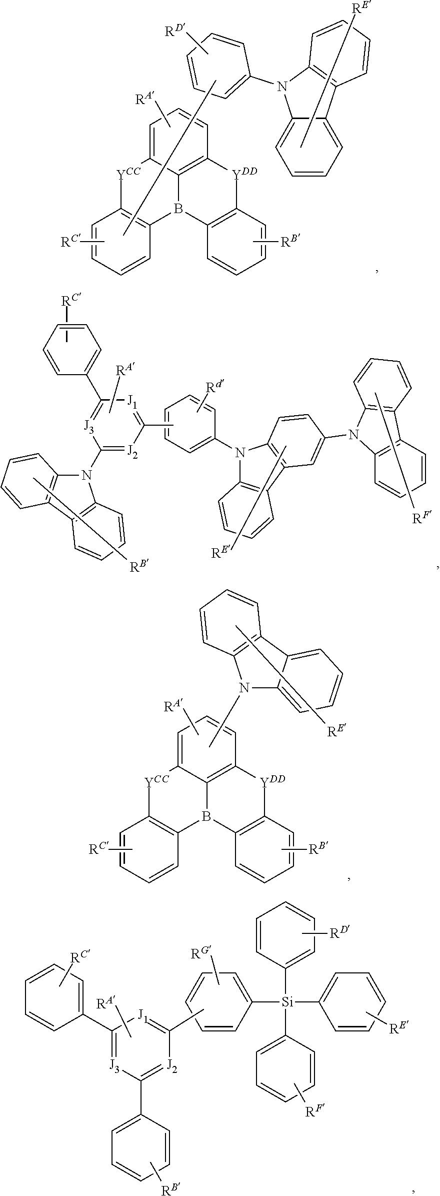 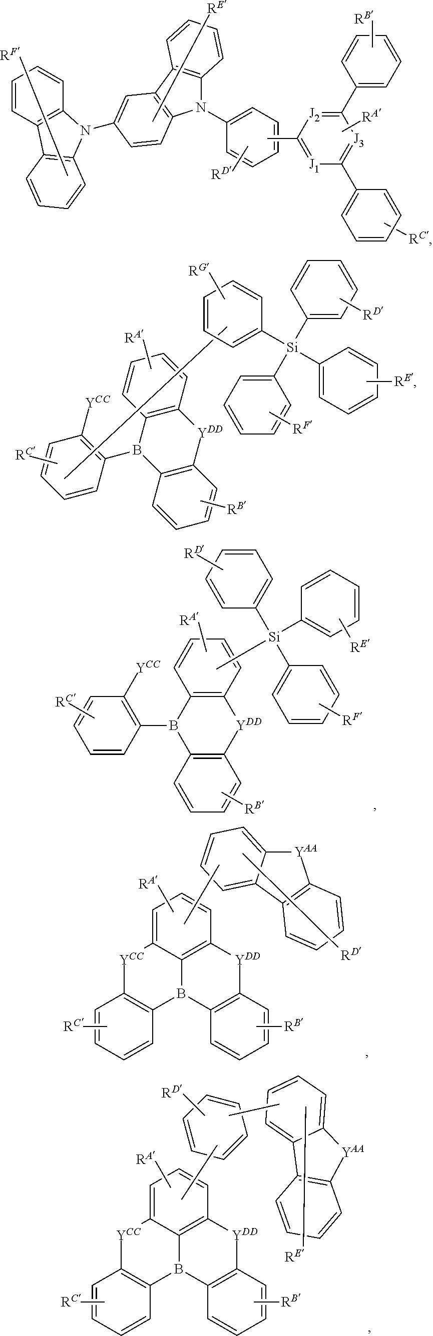 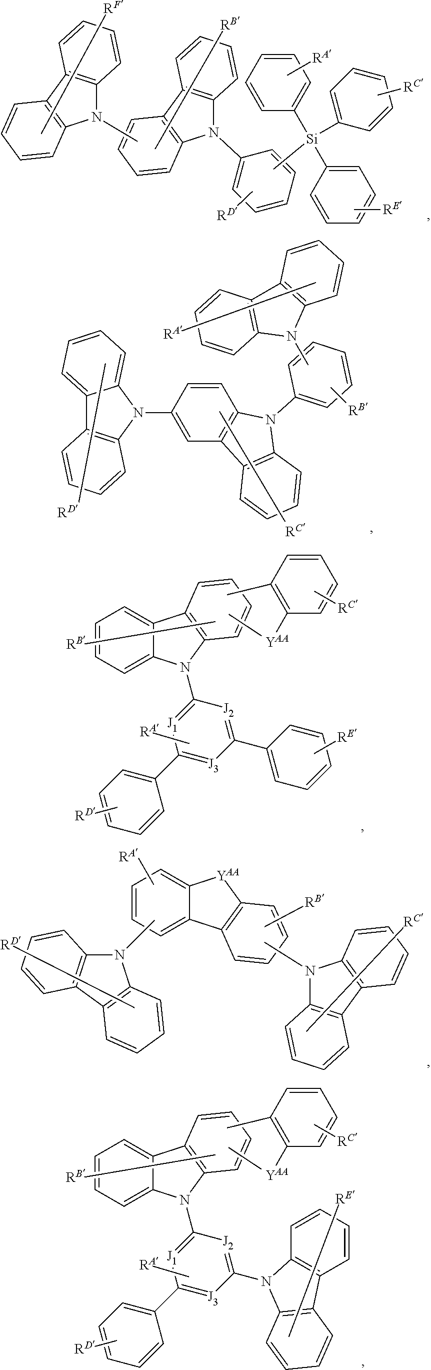 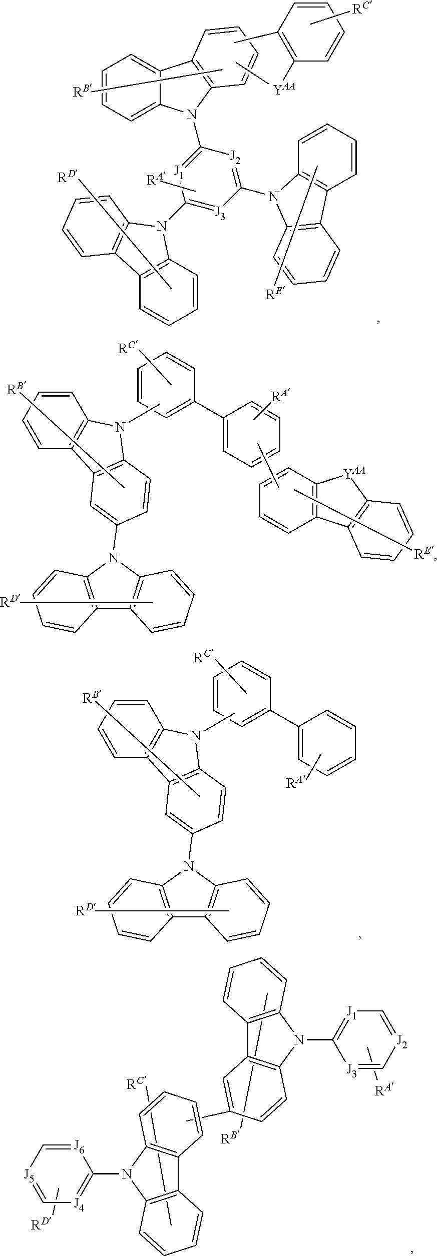 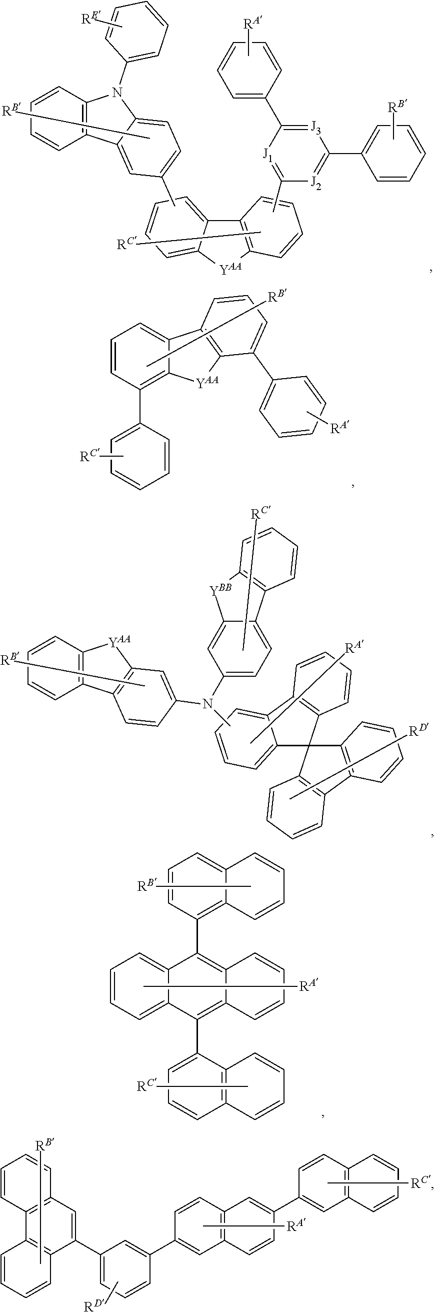 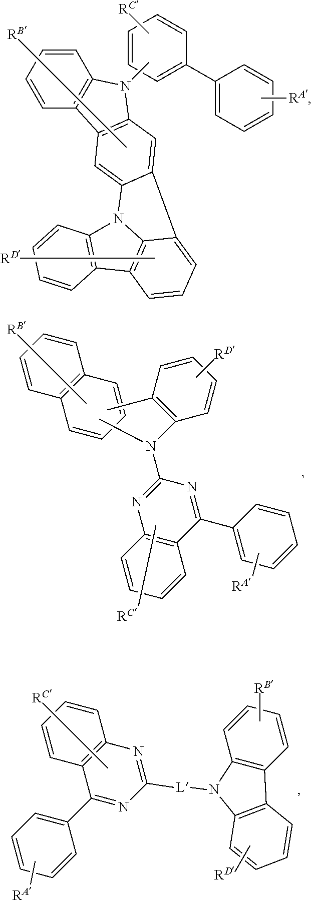 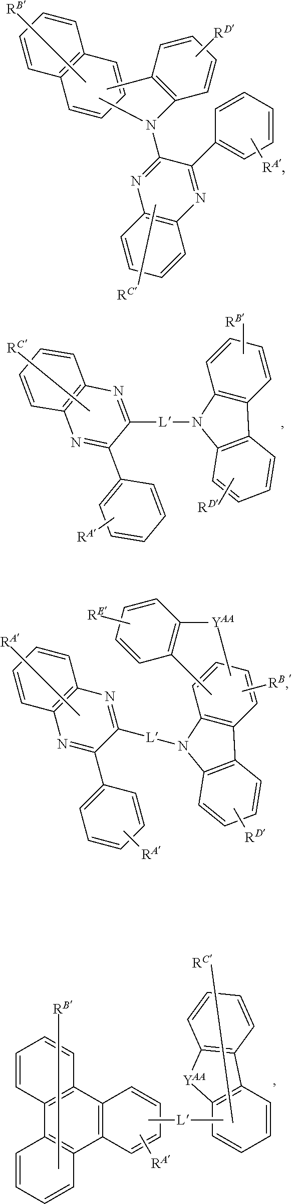 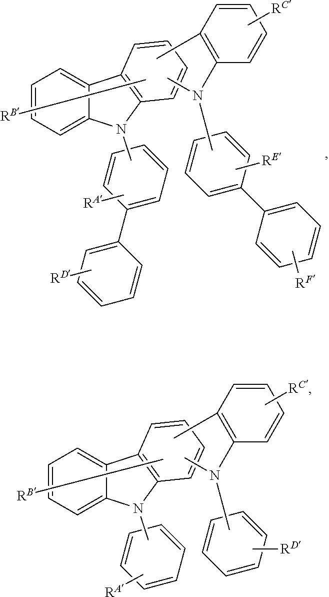 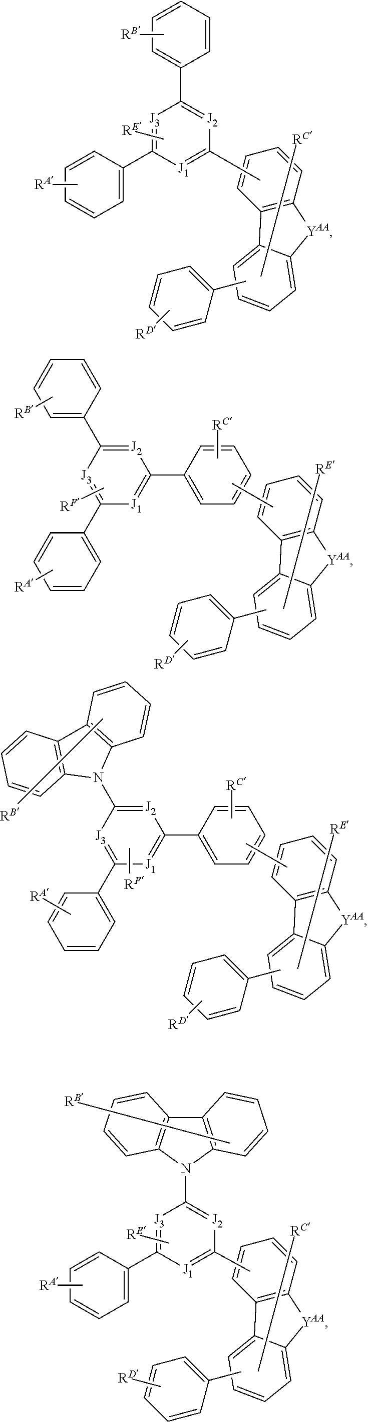 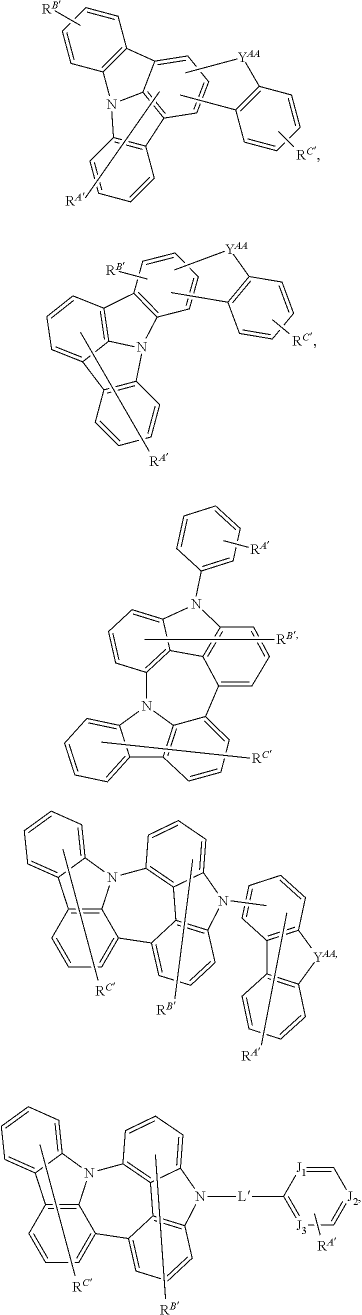 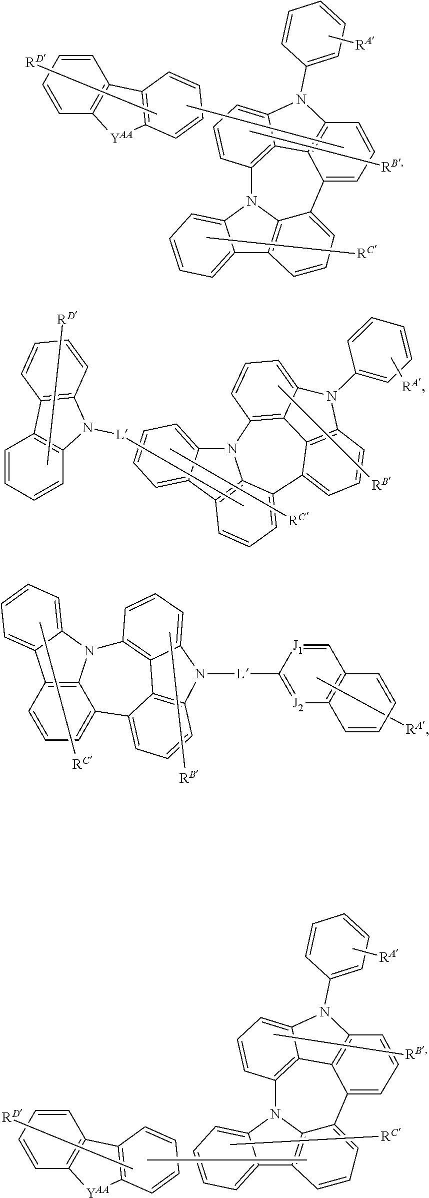 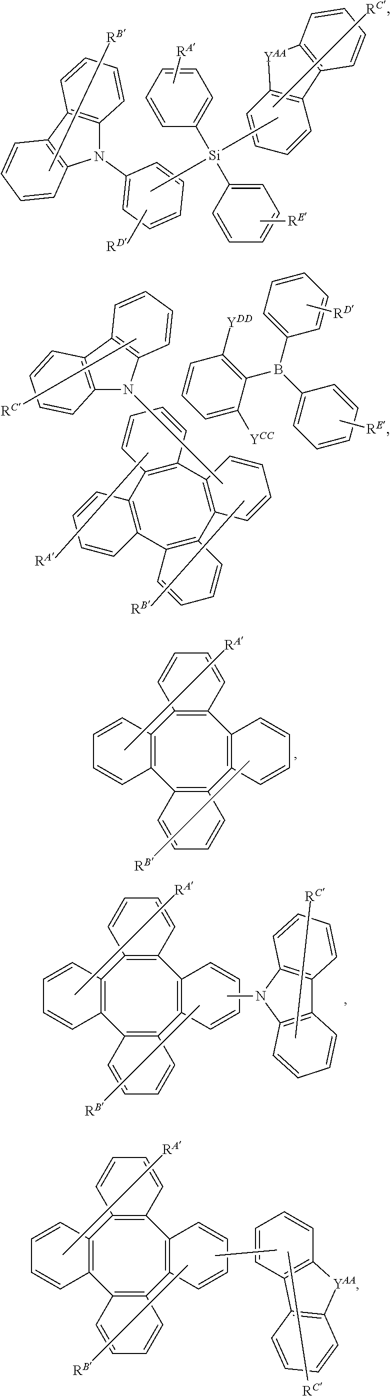 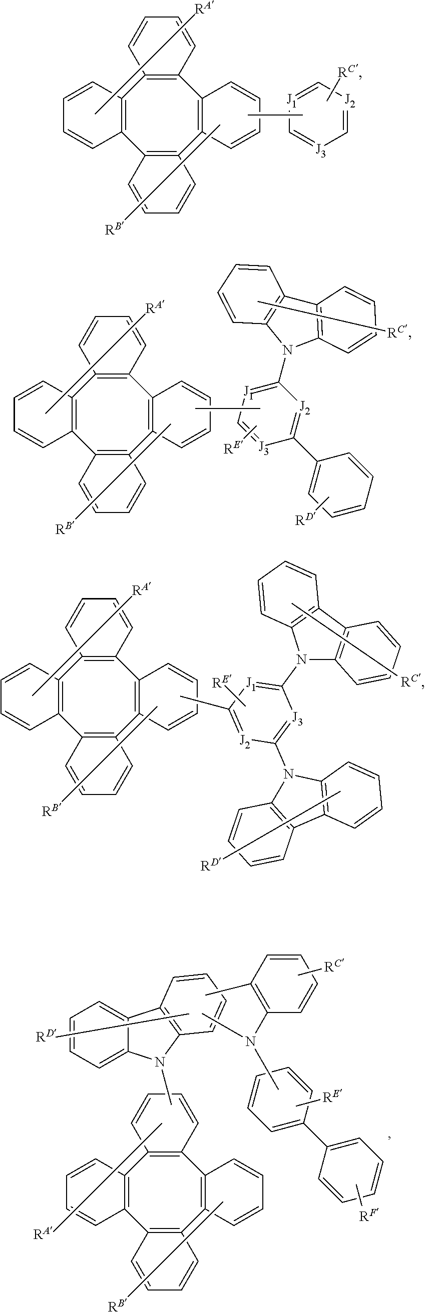 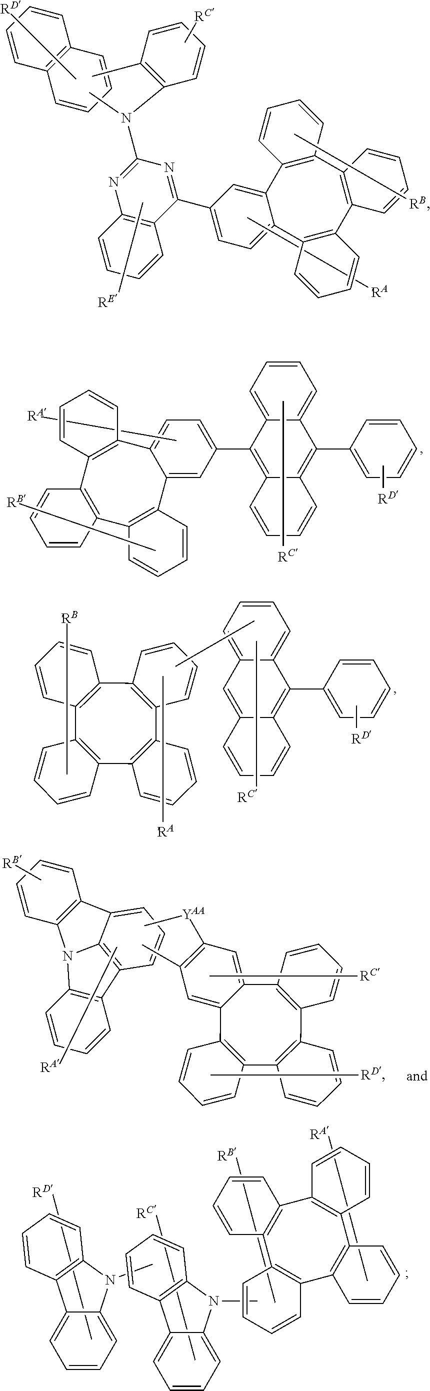
wherein:
each of X¹ to X²⁴ is independently C or N;
L′ is a direct bond or an organic linker;
each Y^{A} is independently selected from the group consisting of absent a bond, O, S, Se, CRR′, SiRR′, GeRR′, NR, BR, BRR′;
each of R^{A′}, R^{B′}, R^{C′}, R^{D′}, R^{E′}, R^{F′}, and R^{G′} independently represents mono, up to the maximum substitutions, or no substitutions;
each of R, R′, R^{A′}, R^{B′}, R^{C′}, R^{D′}, R^{E′}, R^{F′}, and R^{G′} is independently a hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, boryl, and combinations thereof; and
two adjacent of R^{A′}, R^{B′}, R^{C′}, R^{D′}, R^{E′}, R^{F′}, and R^{G′} are optionally joined or fused to form a ring.

14. The OLED of claim 1, wherein the OLED further comprises a second host compound H2.

15. The OLED of claim 1, wherein the compound D1 is a metal coordination complex having a metal-carbon bond; and/or
wherein the compound D1 is a metal coordination complex having a metal-nitrogen bond; and/or
wherein the compound D1 is a metal coordination complex having a metal-oxygen bond; and/or
wherein the metal is Ir or Pt.

16. The OLED of claim 1, wherein the compound D1 has the formula of M(L¹)ₓ(L²)_{y}(L³)_{z};
wherein L¹, L², and L³ can be the same or different;
wherein x is 1, 2, or 3;
wherein y is 0, 1, or 2;
wherein z is 0, 1, or 2;
wherein x+y+z is the oxidation state of the metal M;
wherein L¹ is selected from the group consisting of the structures of the following LIST 1: 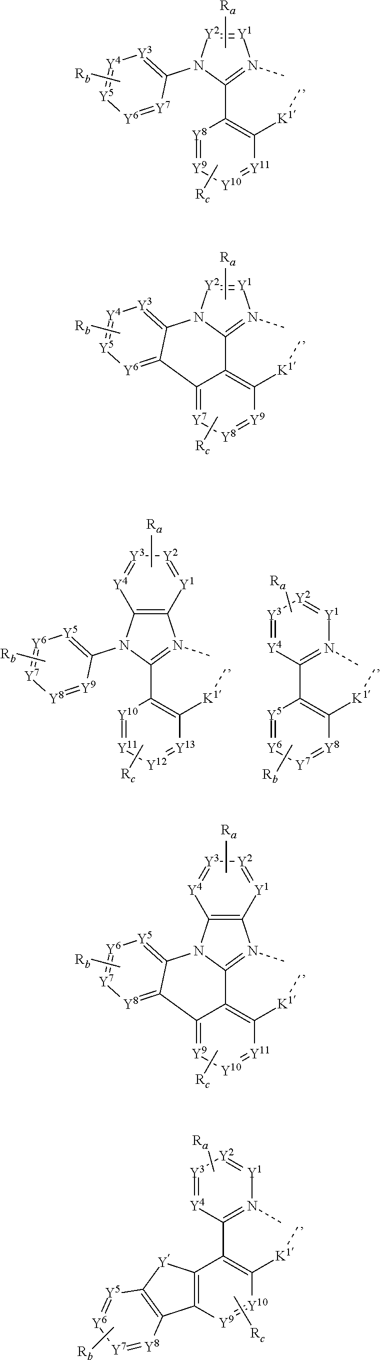 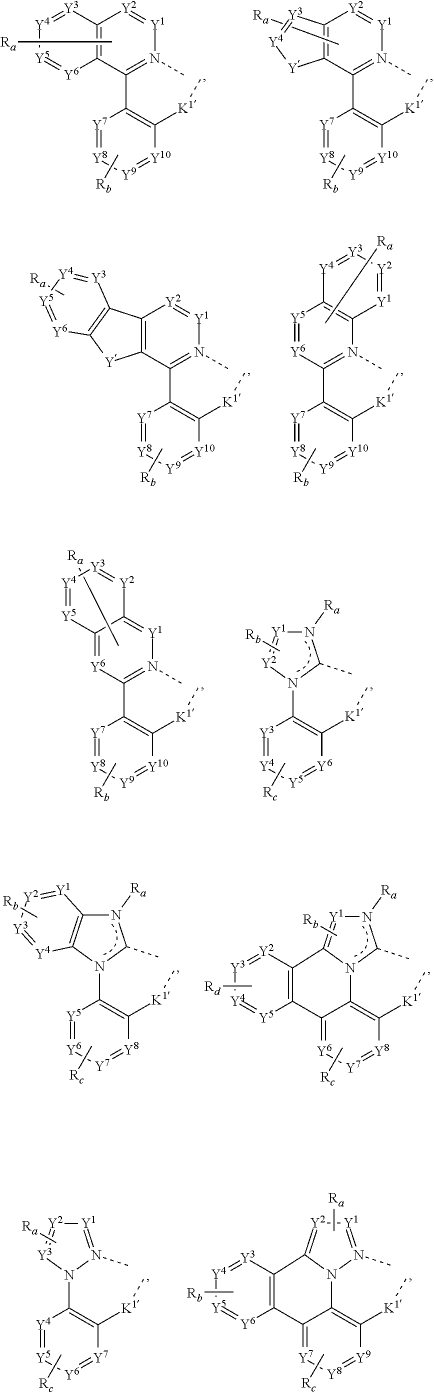 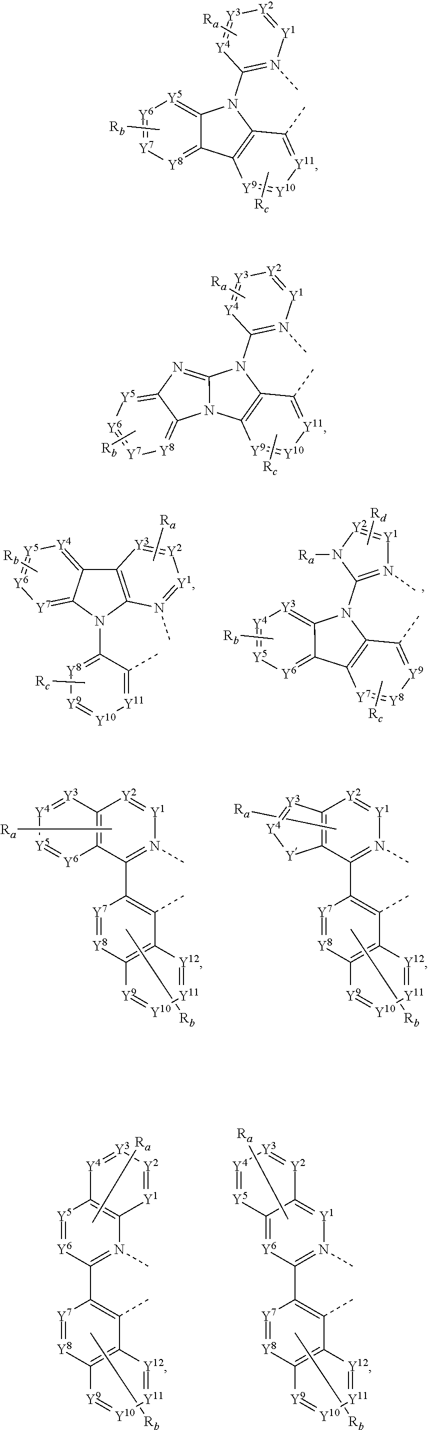 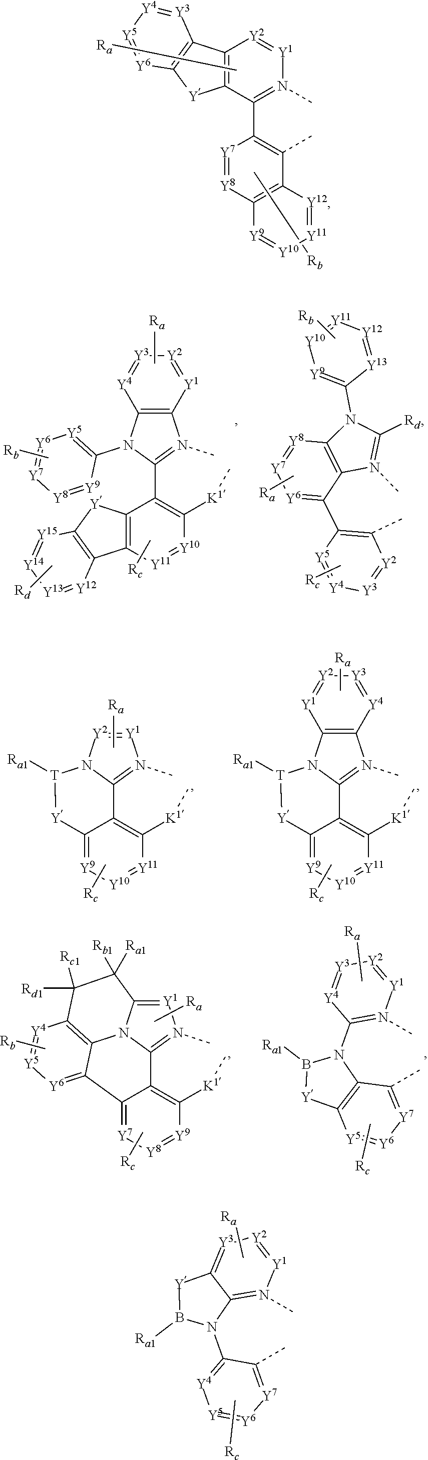 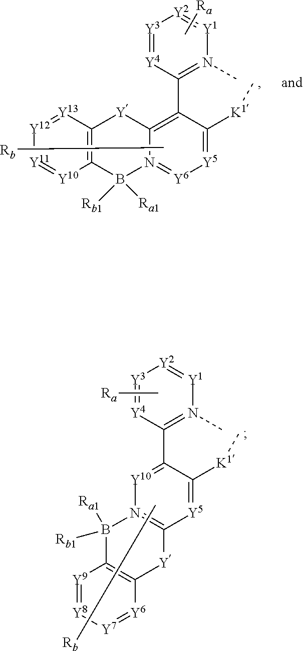
wherein L² and L³ are independently selected from the group consisting of 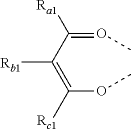
and the structures of the LIST 1;
wherein:
T is selected from the group consisting of B, Al, Ga, and In;
K^{1′} is a direct bond or is selected from the group consisting of NRₑ, PRₑ, O, S, and Se;
each Y¹ to Y¹³ are independently selected from the group consisting of carbon and nitrogen;
Y′ is selected from the group consisting of BRₑ, NRₑ, P Rₑ, O, S, Se, C═O, S═O, SO₂, CRₑR_{f}, SiRₑR_{f}, and GeRₑR_{f};
Rₑ and R_{f} can be fused or joined to form a ring;
each Rₐ, R_{b}, R_{c}, and R_{d} can independently represent from mono to the maximum possible number of substitutions, or no substitution;
each Rₐ₁, R_{b1}, R_{c1}, R_{d1}, Rₐ, R_{b}, R_{c}, R_{d}, Rₑ, and R_{f} is independently a hydrogen or a substituent selected from the group consisting of deuterium, halogen, alkyl, cycloalkyl, heteroalkyl, heterocycloalkyl, boryl, arylalkyl, alkoxy, aryloxy, amino, silyl, germyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof; and
any two adjacent substituents of Rₐ₁, R_{b1}, R_{c1}, R_{d1}, Rₐ, R_{b}, R_{c}, and R_{d} can be fused or joined to form a ring or form a multidentate ligand.

17. The OLED of claim 1, wherein D1 is selected from the group consisting of: 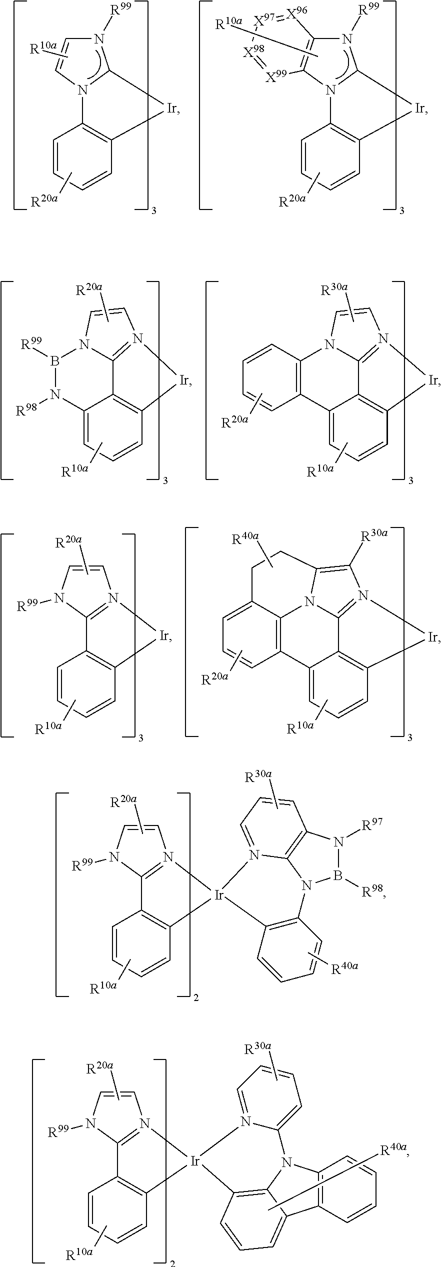 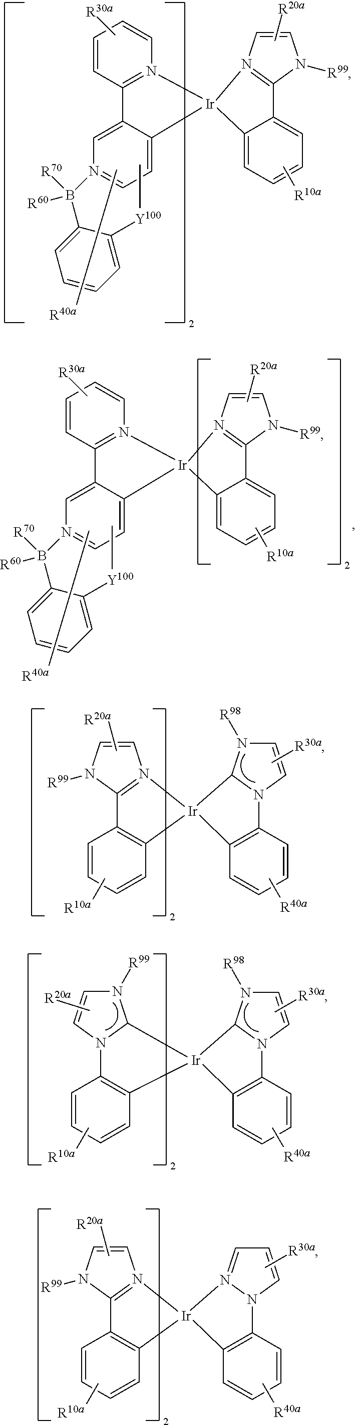 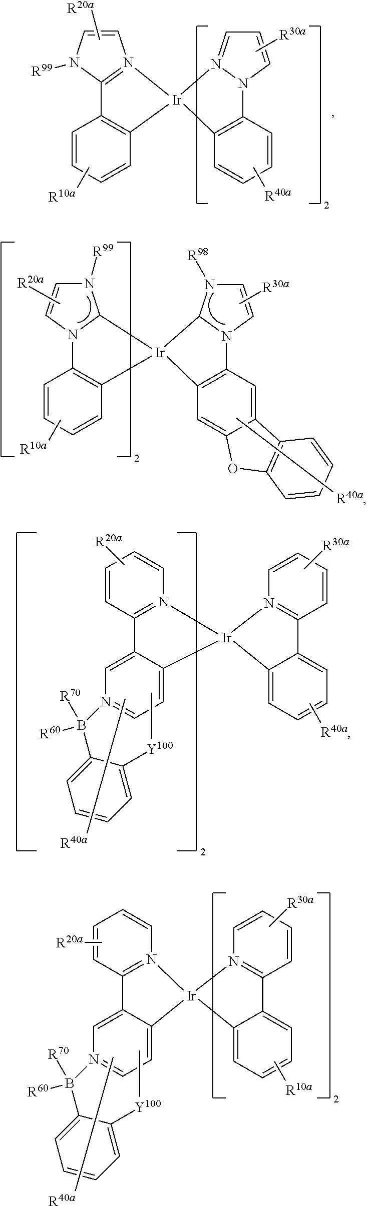 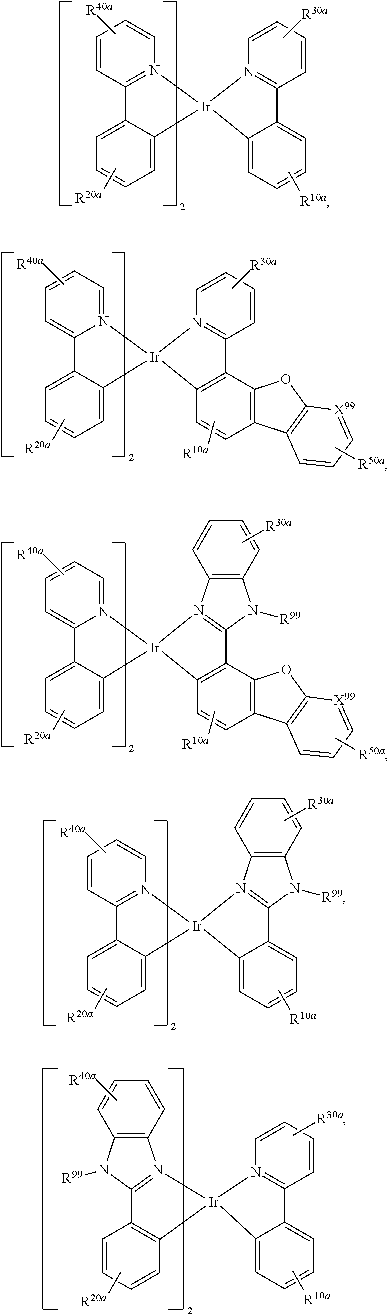 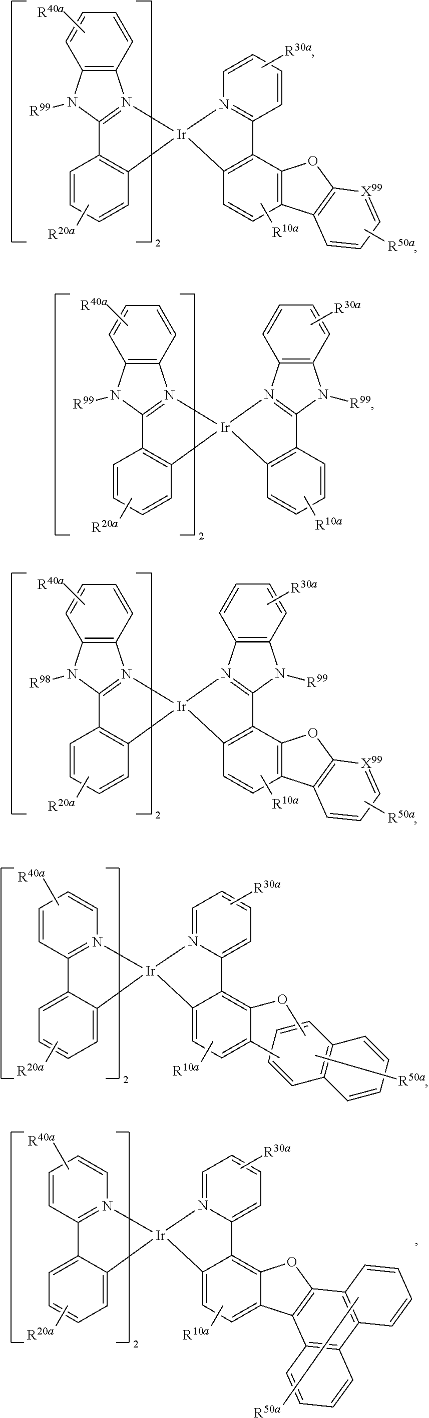 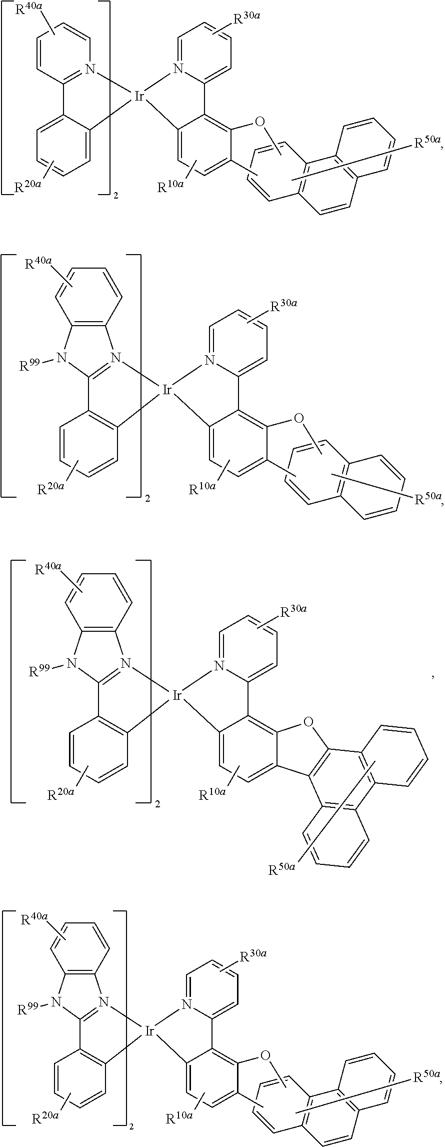 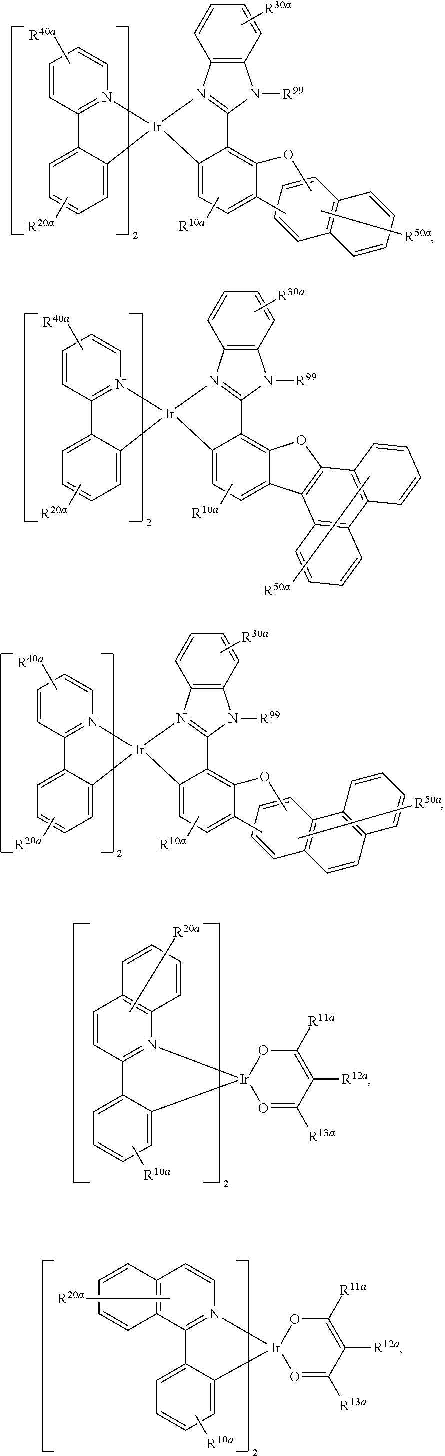 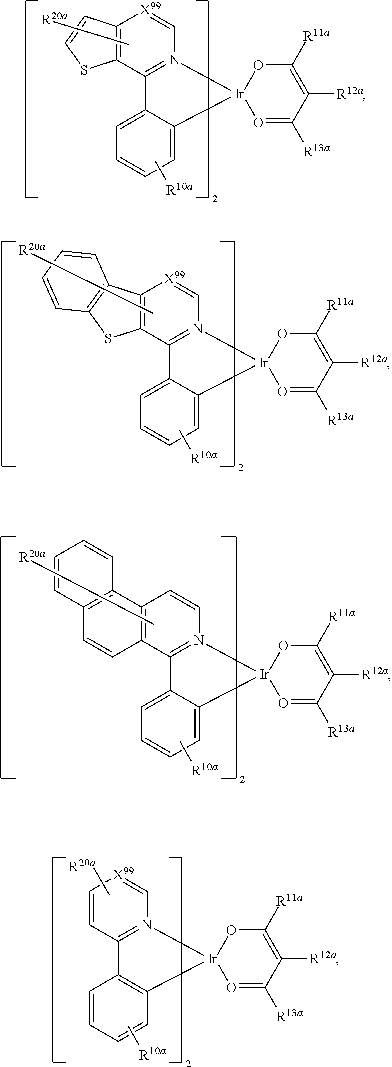 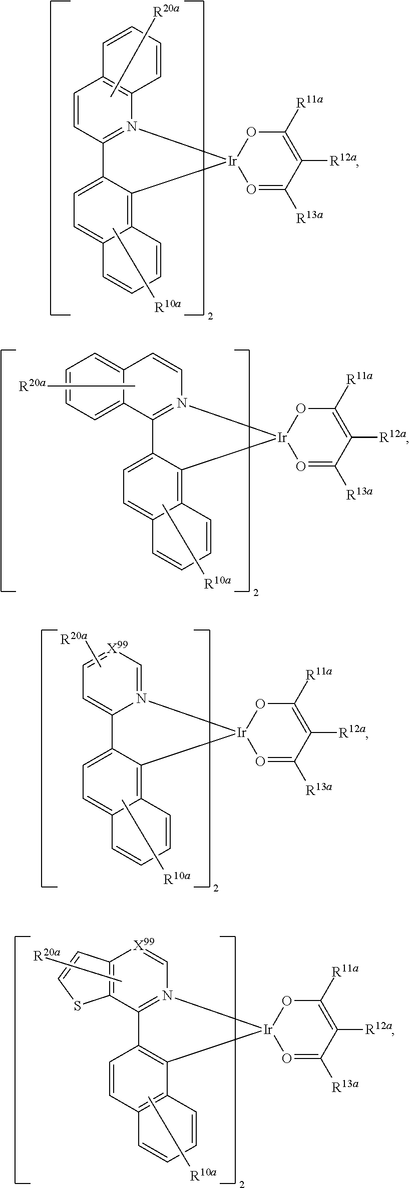 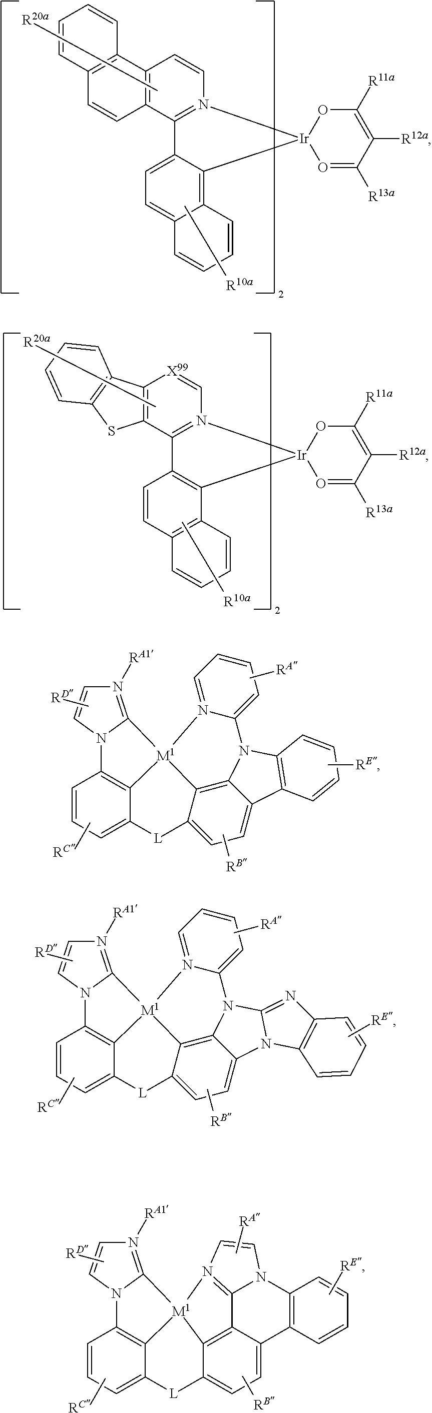 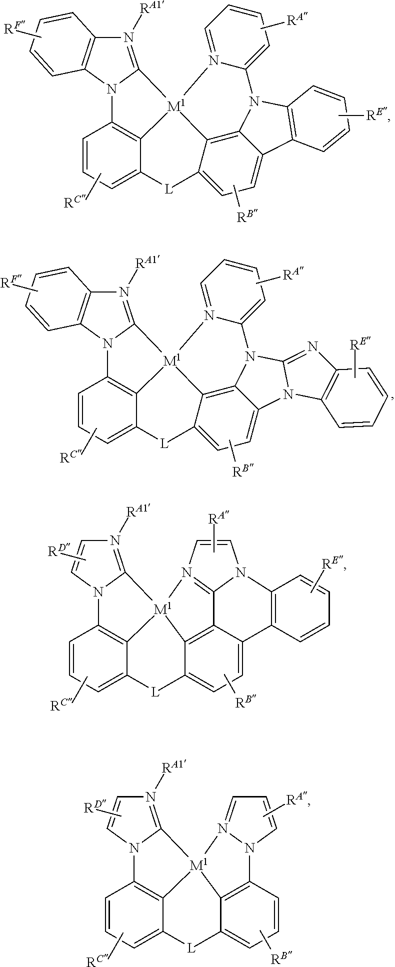 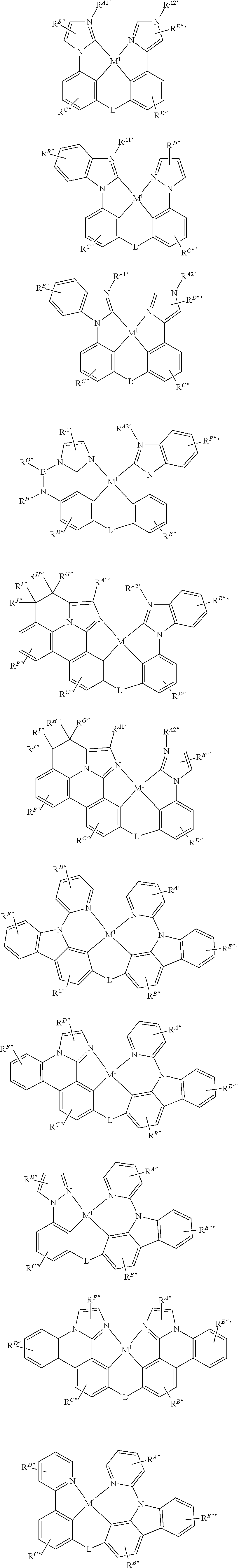 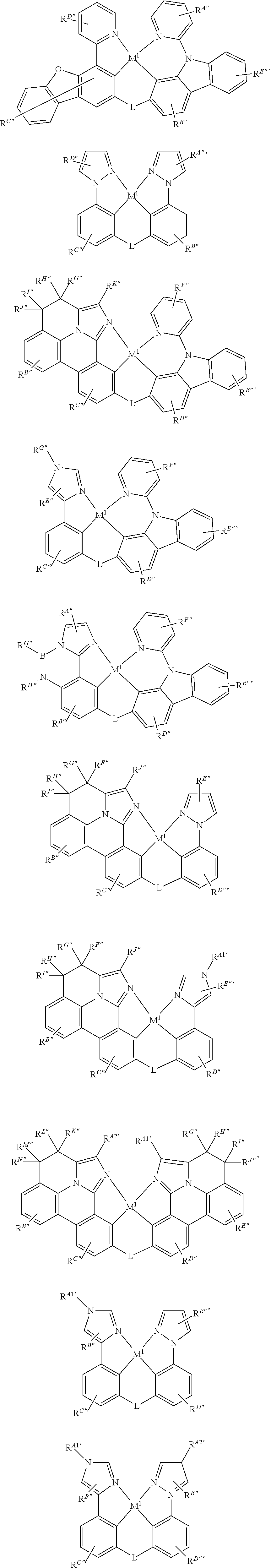 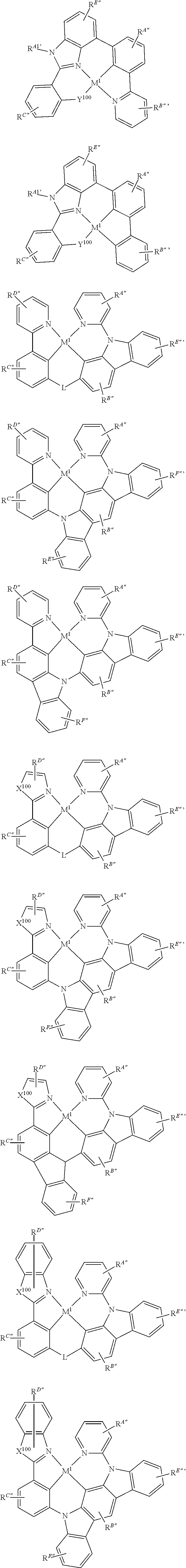 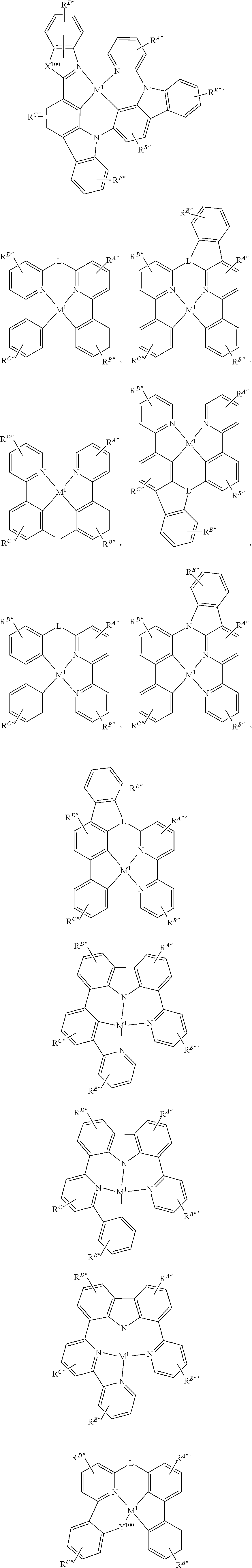 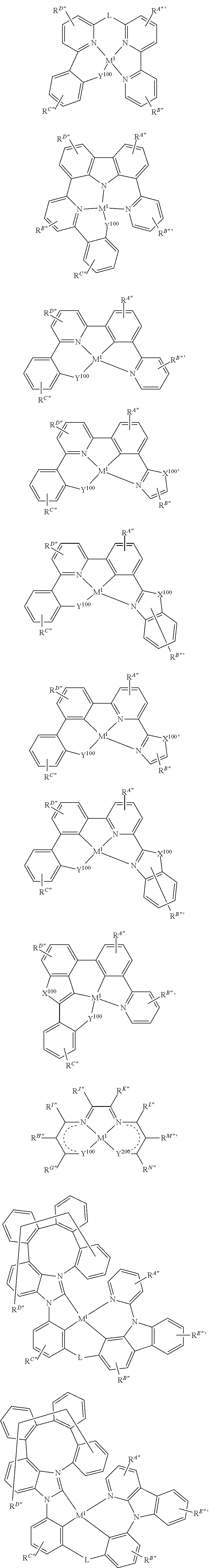 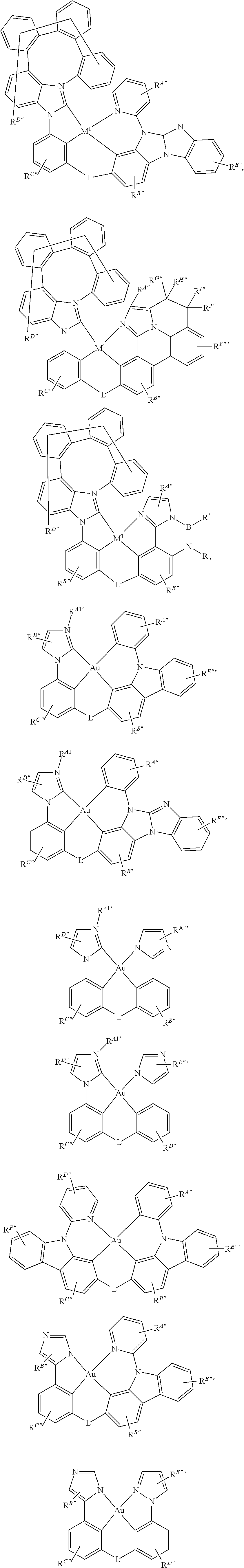 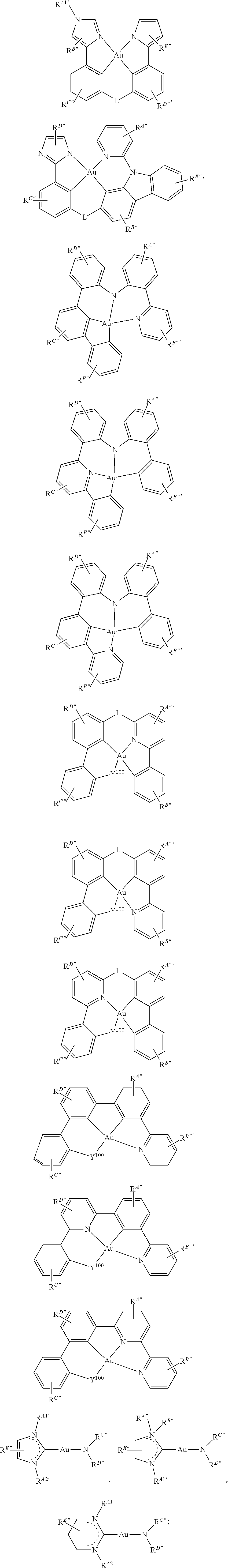
wherein:
each of X⁹⁶ to X⁹⁹ is independently C or N;
each of Y¹⁰⁰ and Y²⁰⁰ is independently selected from the group consisting of a NR″, O, S, and Se;
L is independently selected from the group consisting of a direct bond, BR″, BR″R′″, NR″, PR″, O, S, Se, C═O, C═S, C═Se, C═NR″, C═CR″R′″, S═O, SO₂, CR″, CR″R′″, SiR″R′″, GeR″R′″, alkyl, cycloalkyl, aryl, heteroaryl, and combinations thereof;
X¹⁰⁰ for each occurrence is selected from the group consisting of O, S, Se, NR″, and CR″R′″;
each R^{10a}, R^{20a}, R^{30a}, R^{40a}, R^{50a}, R^{A″}, R^{B″}, R^{C″}, R^{D″}, R^{E″}, and R^{F}″ independently represents mono-, up to the maximum substitutions, or no substitutions;
each R, R′, R″, R′″, R^{10a}, R^{11a}, R^{12a}, R^{13a}, R^{20a}, R^{30a}, R^{40a}, R^{50a}, R⁶⁰, R⁷⁰, R⁹⁷, R⁹⁸, R⁹⁹, R^{A1′}, R^{A2′}, R^{A}″ R^{B″}, R^{C″}, R^{D″}, R^{E″}, R^{F″}, R^{G″}, R^{H″}, R^{I″}, R^{J″}, R^{K″}, R^{L″}, R^{M″}, and R^{N}″ is independently a hydrogen or a substituent selected from the group consisting of deuterium, halogen, alkyl, cycloalkyl, heteroalkyl, heterocycloalkyl, boryl, arylalkyl, alkoxy, aryloxy, amino, silyl, germyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof; and any two substituents can be joined or fused into a ring.

18. The OLED of claim 1, wherein the compound D1 is selected from the group consisting of:

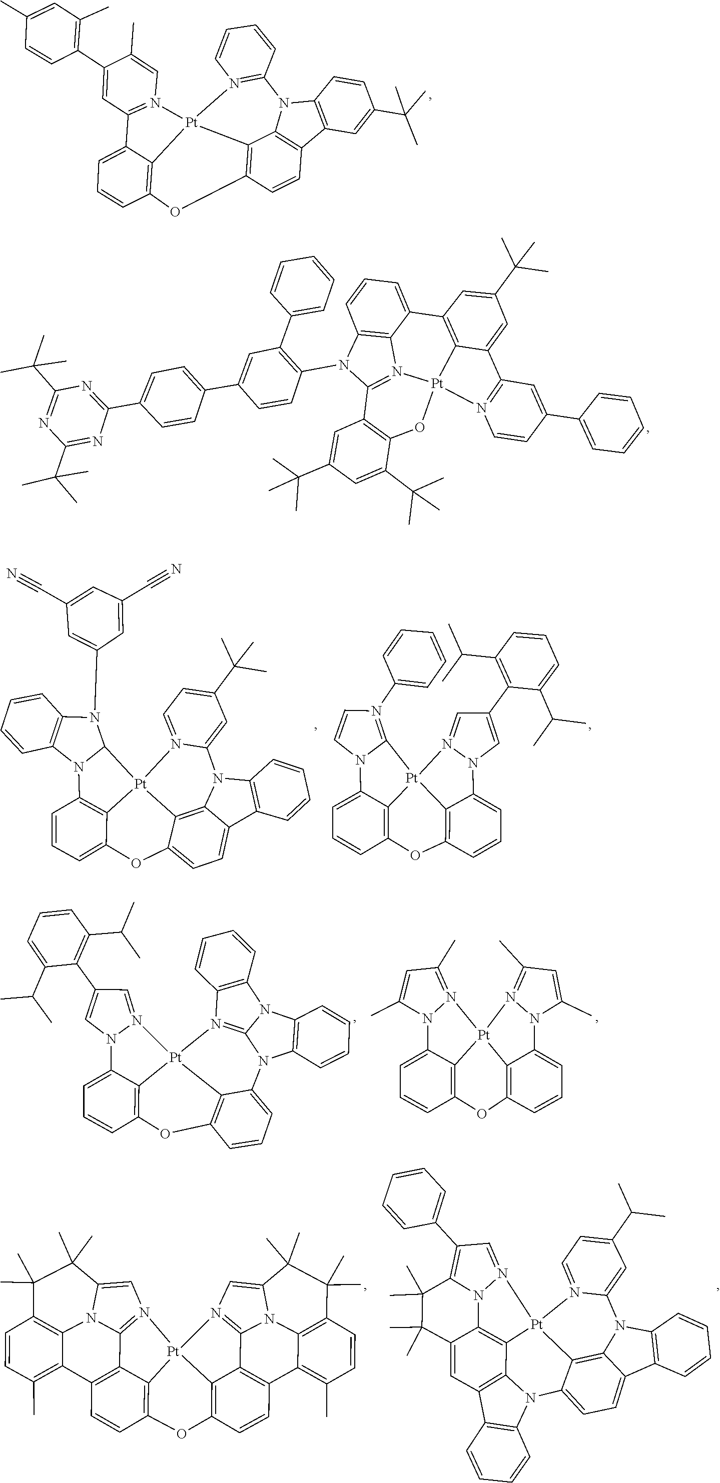

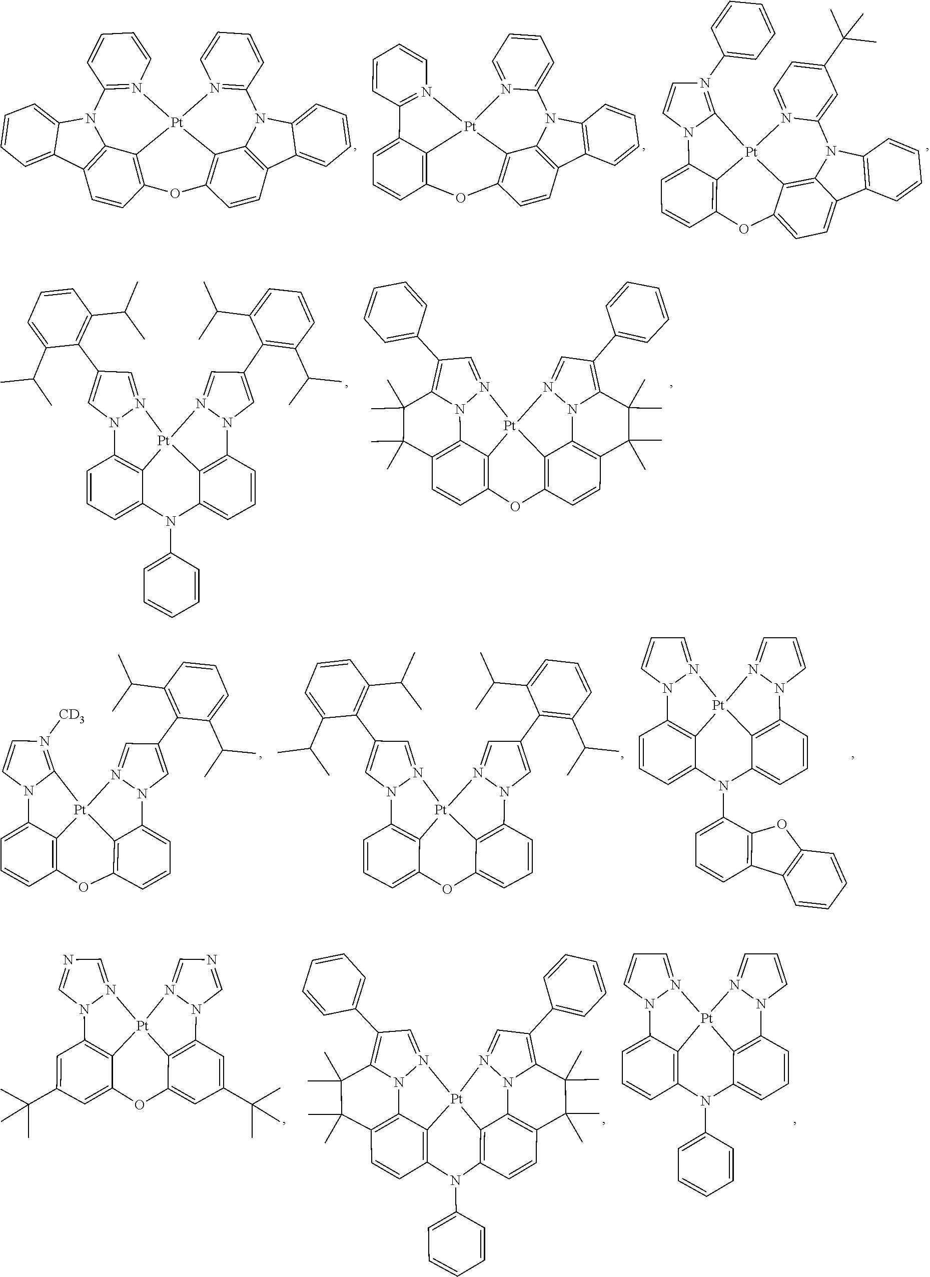

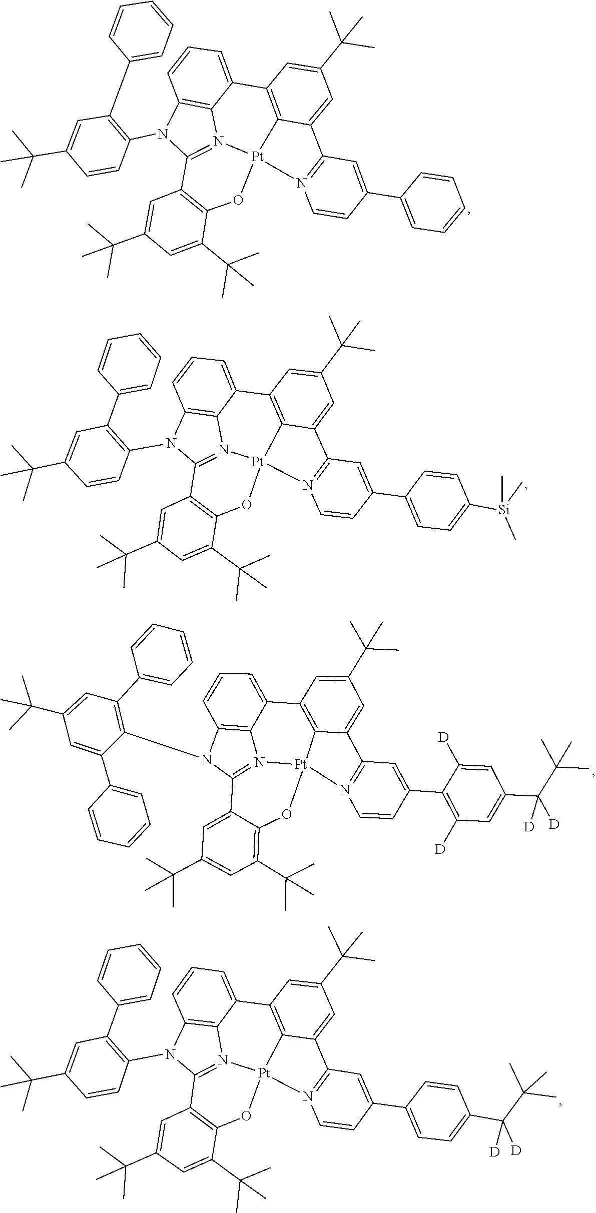

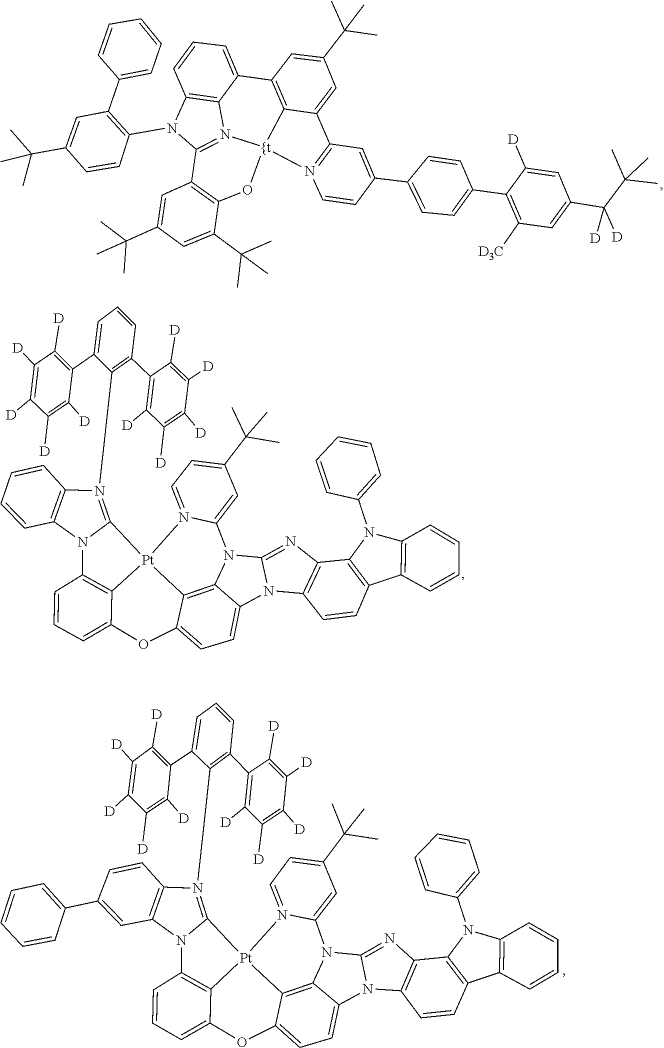

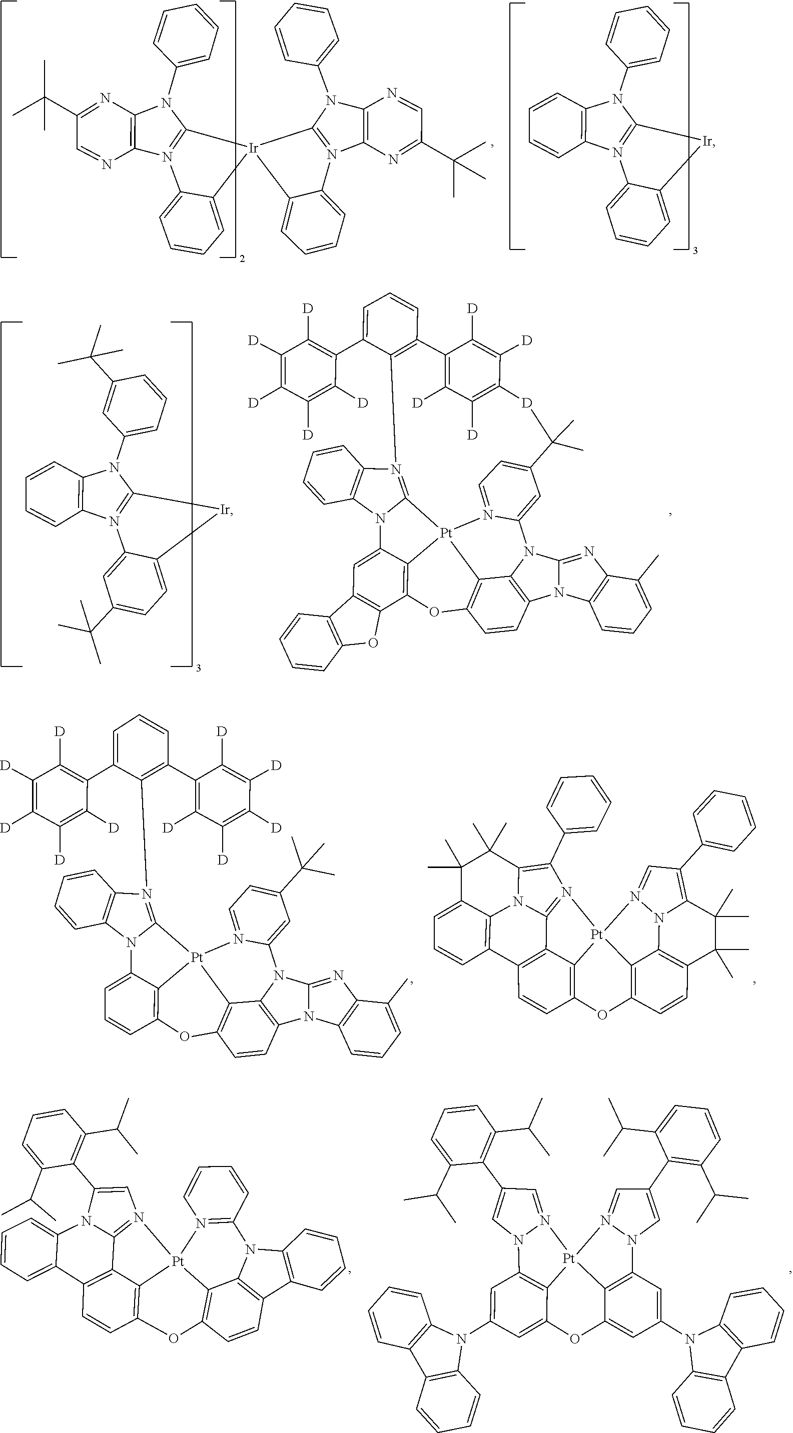

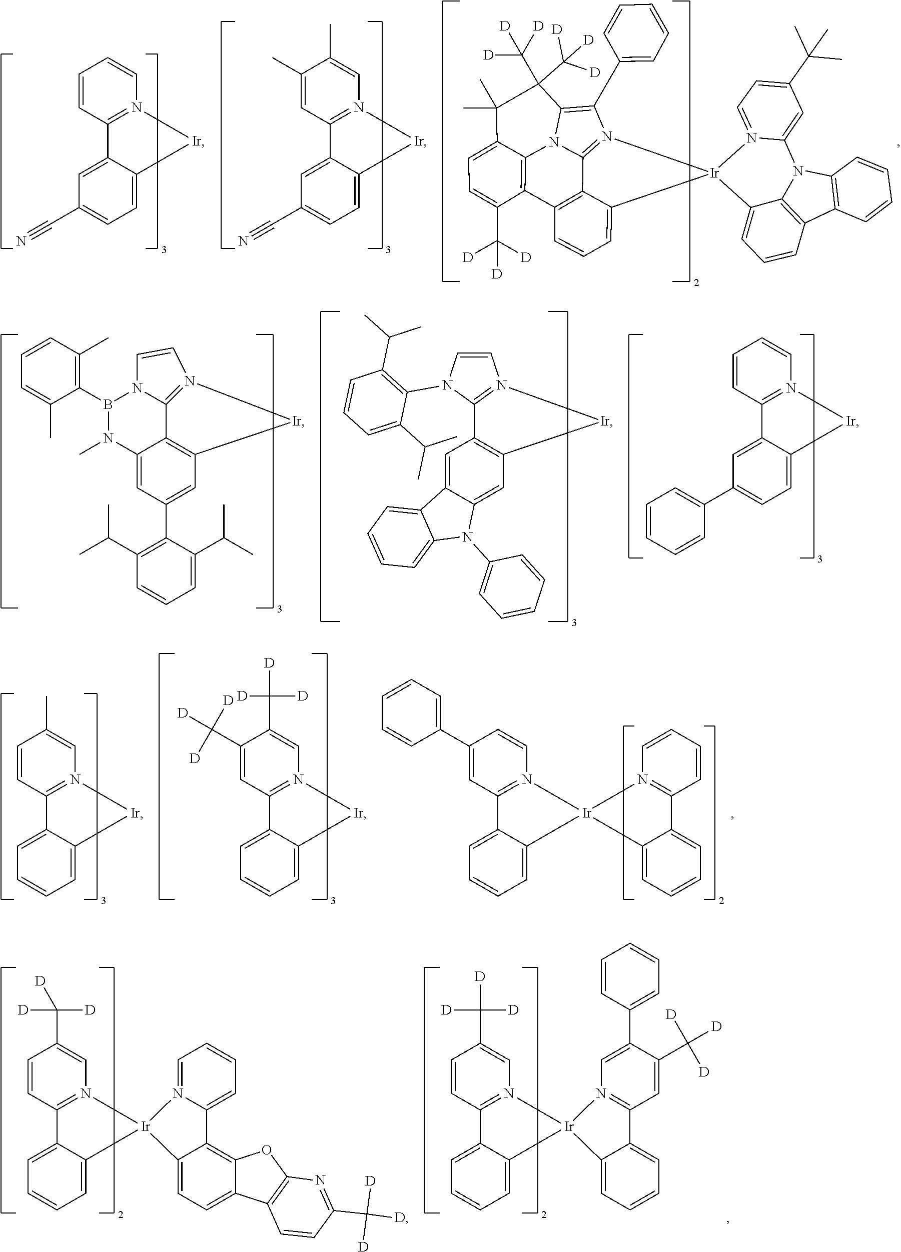

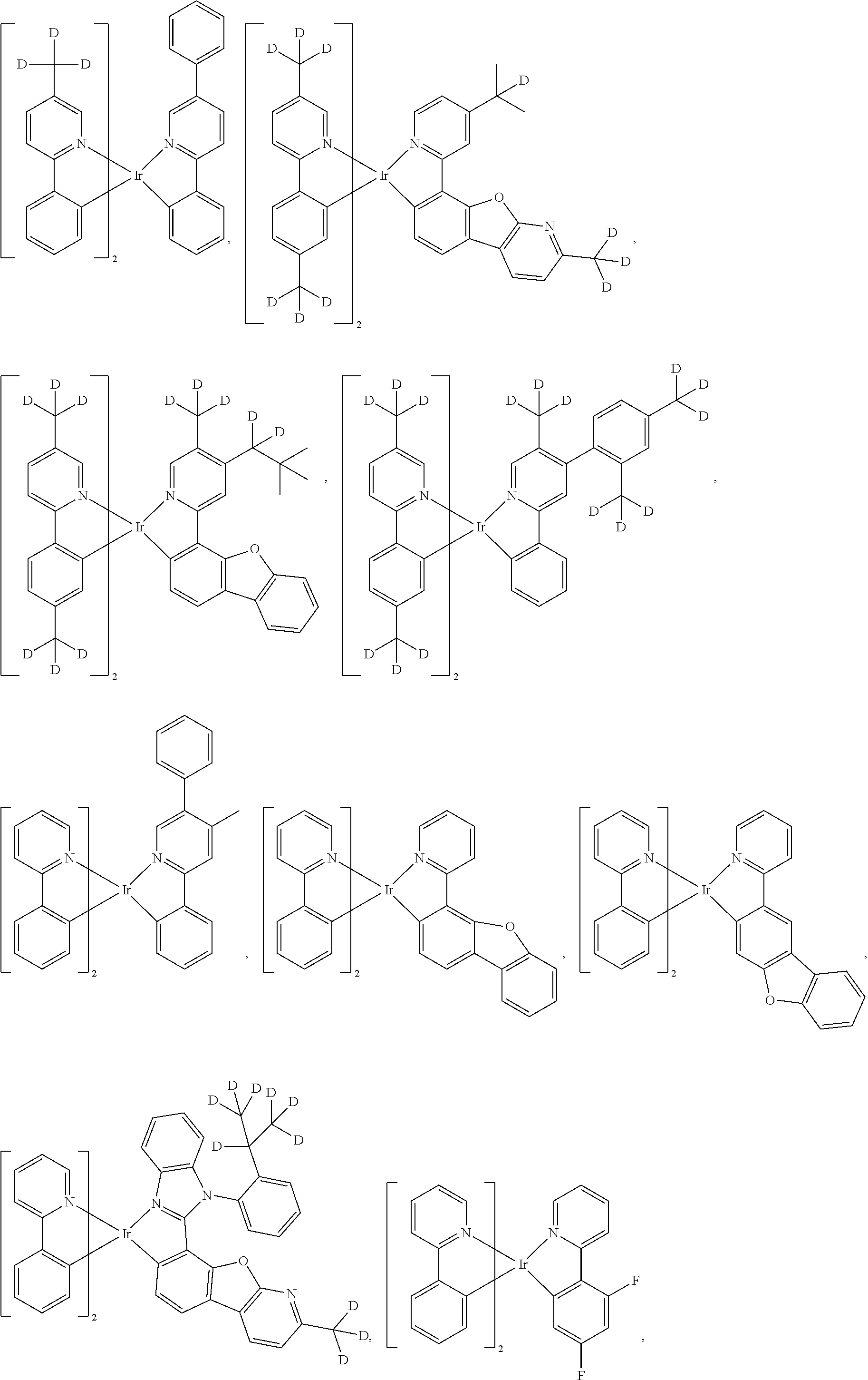

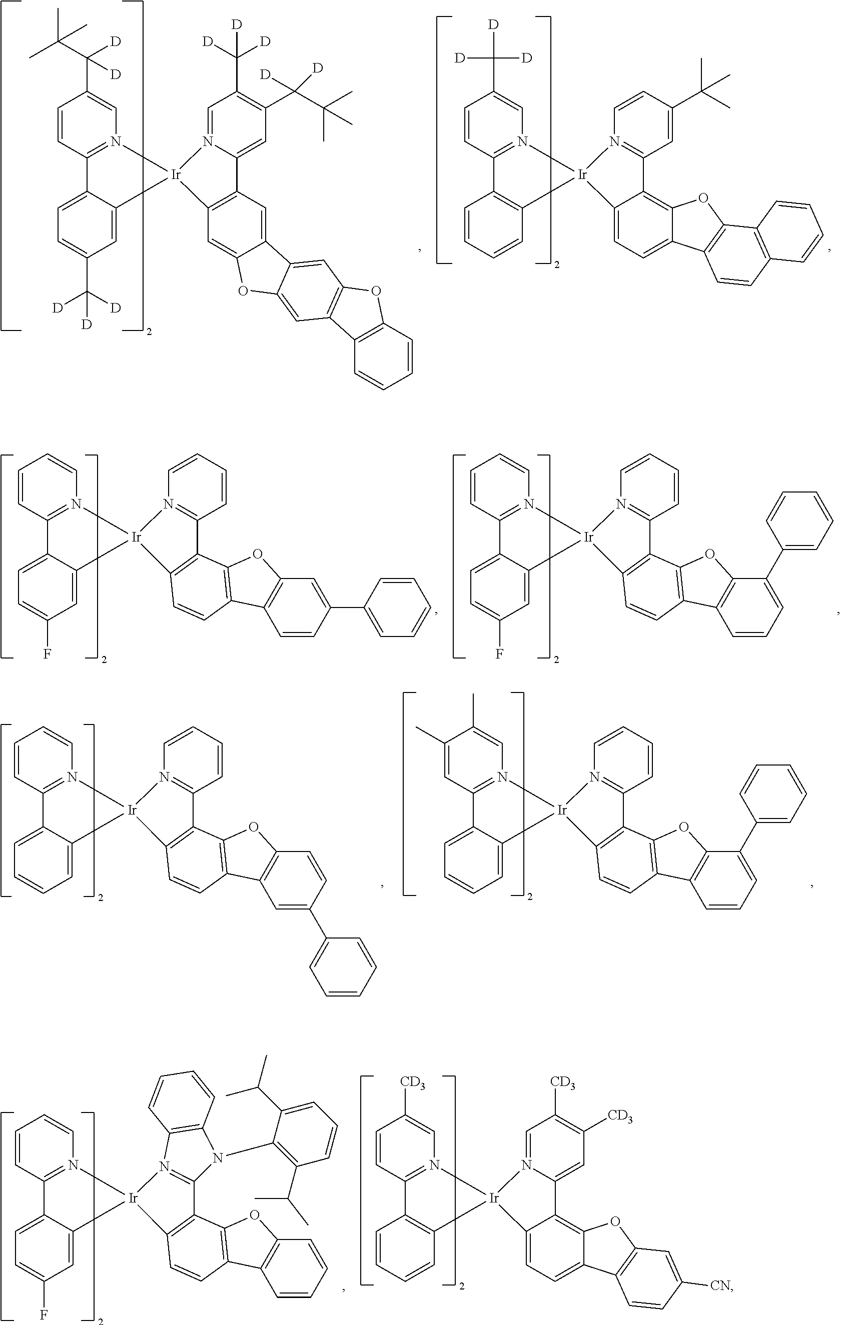

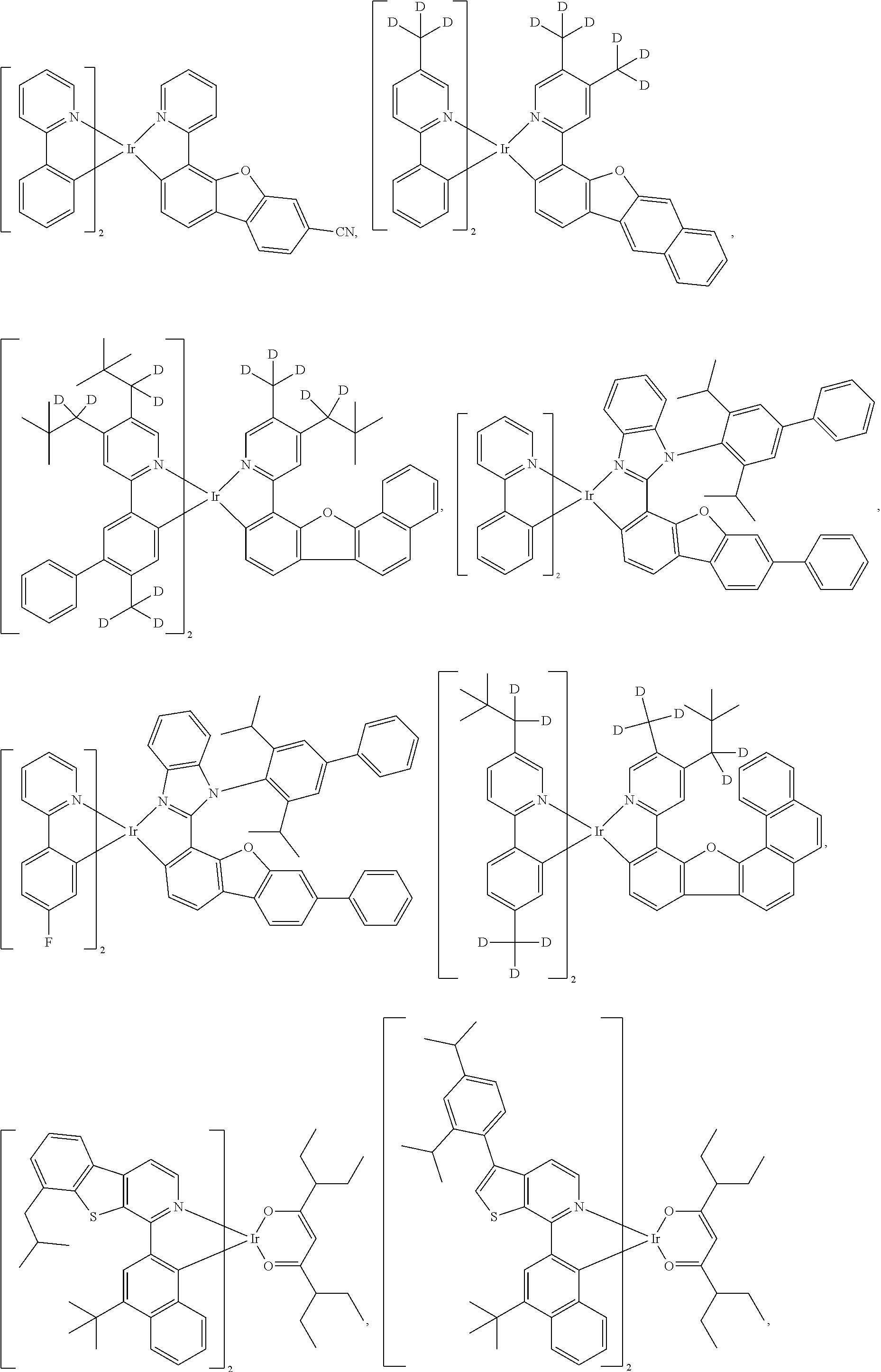

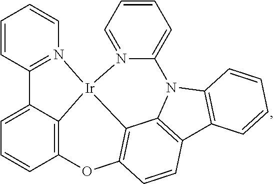

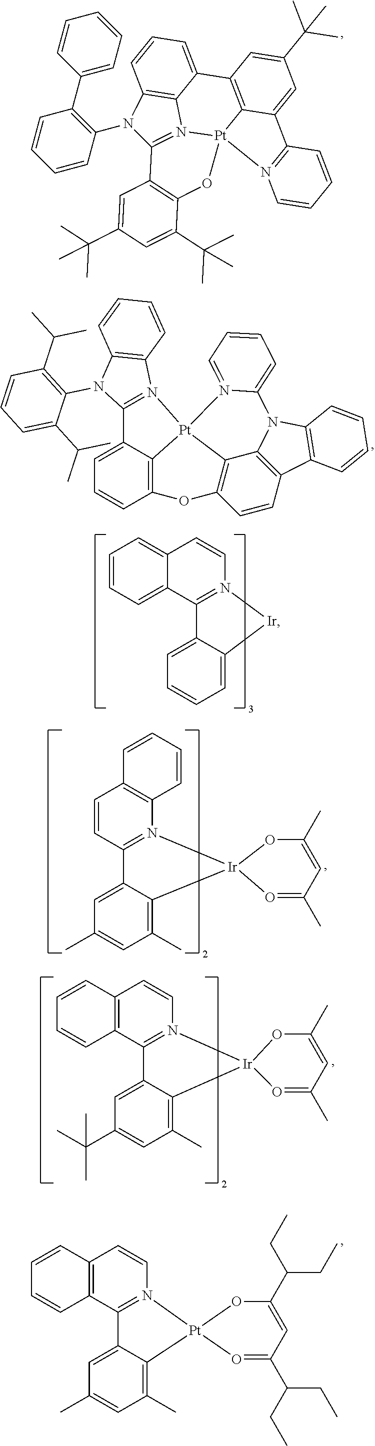

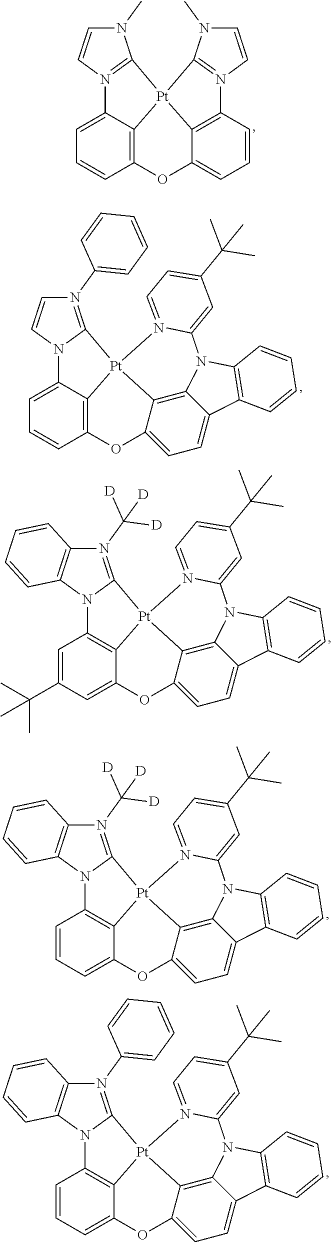

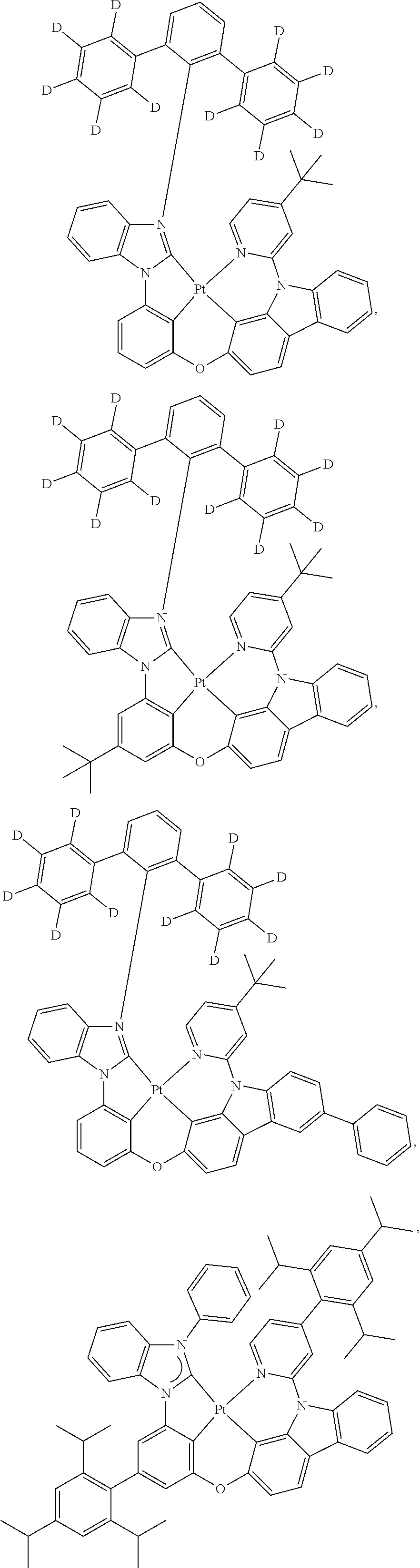

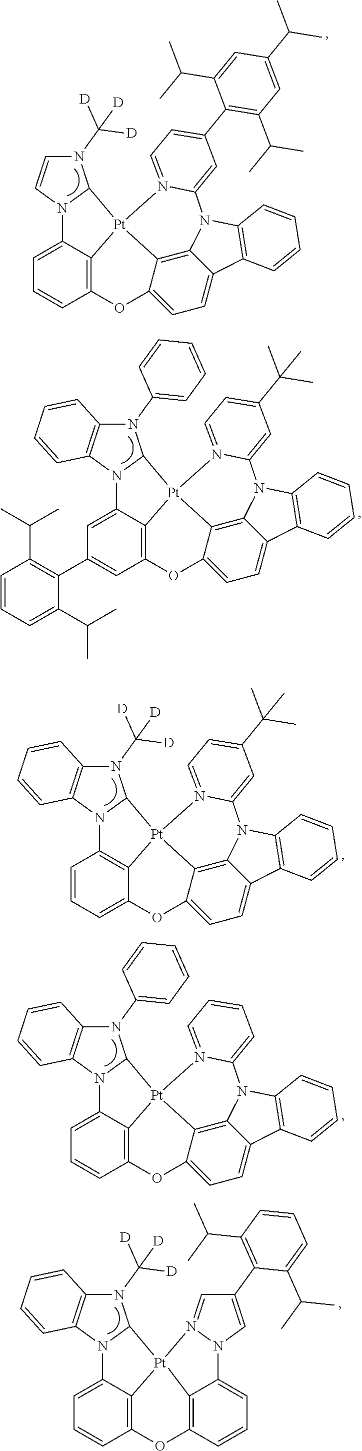

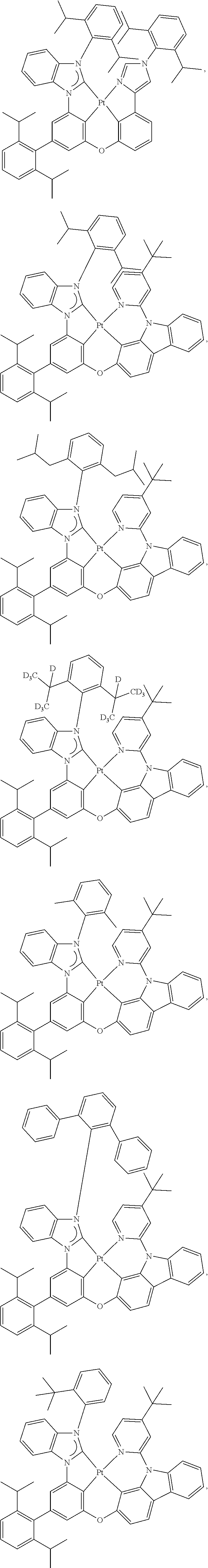

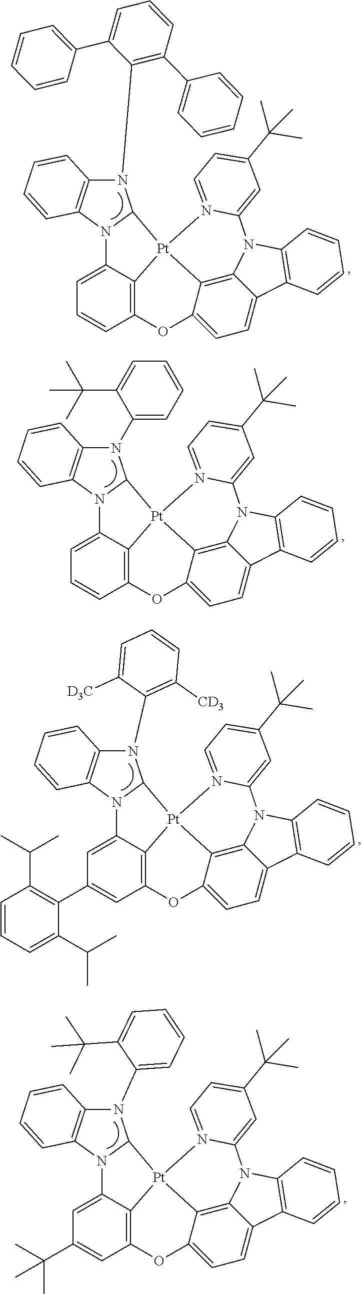

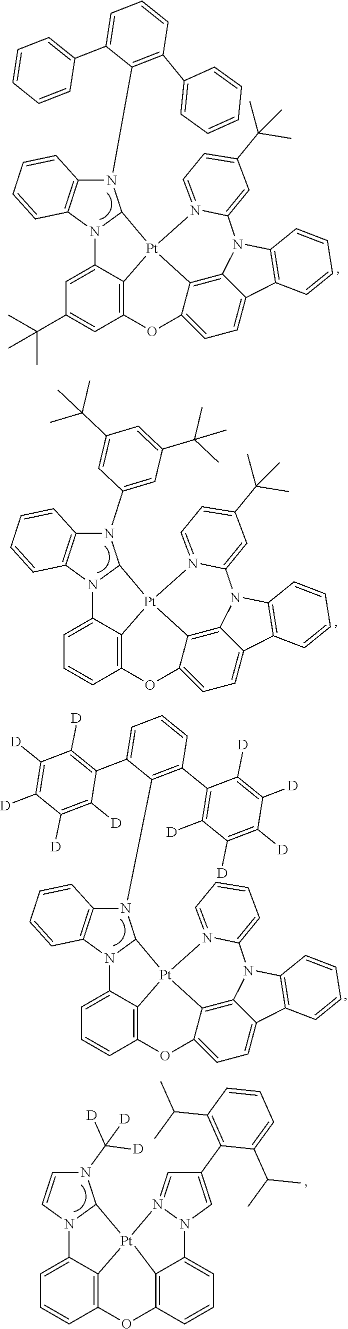

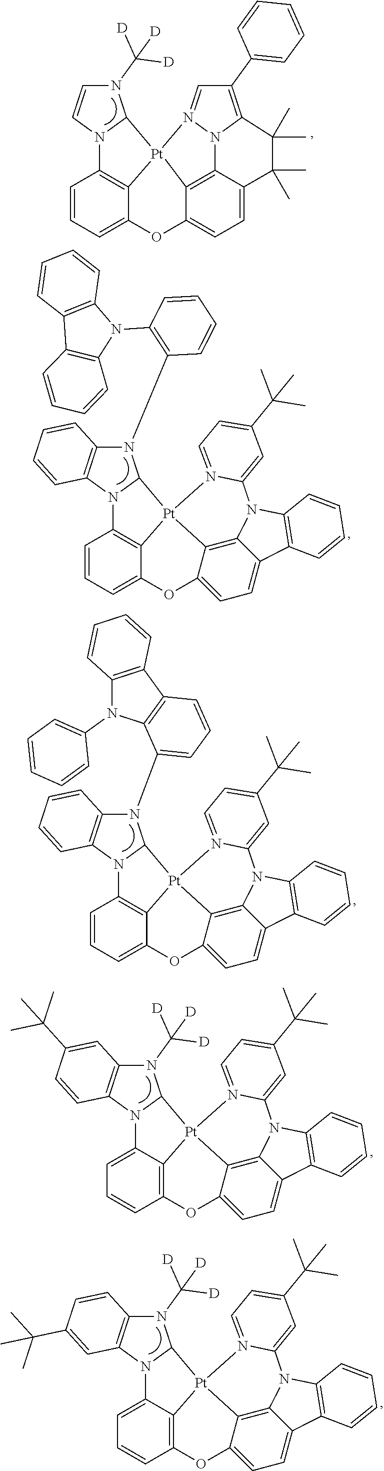

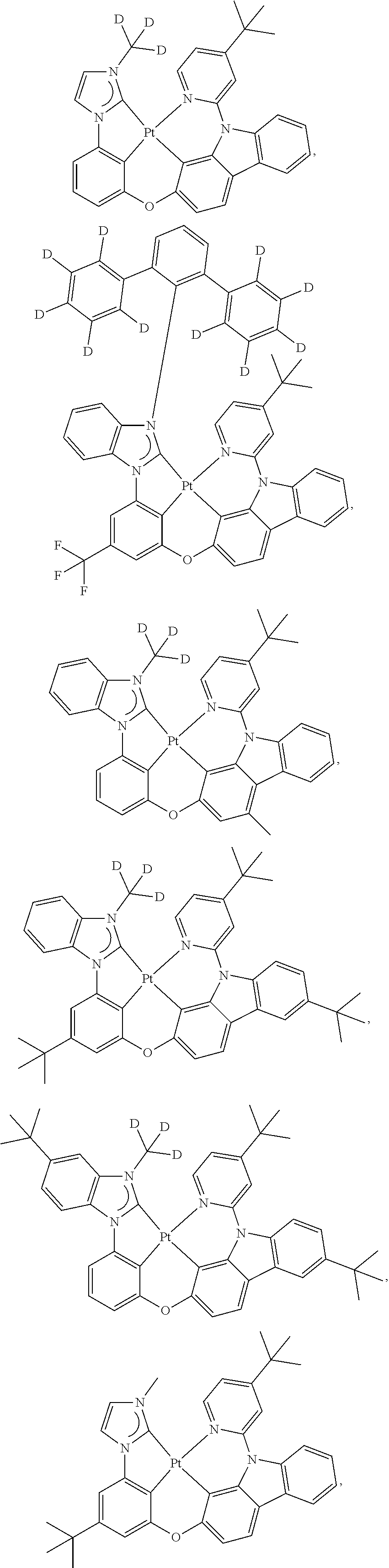

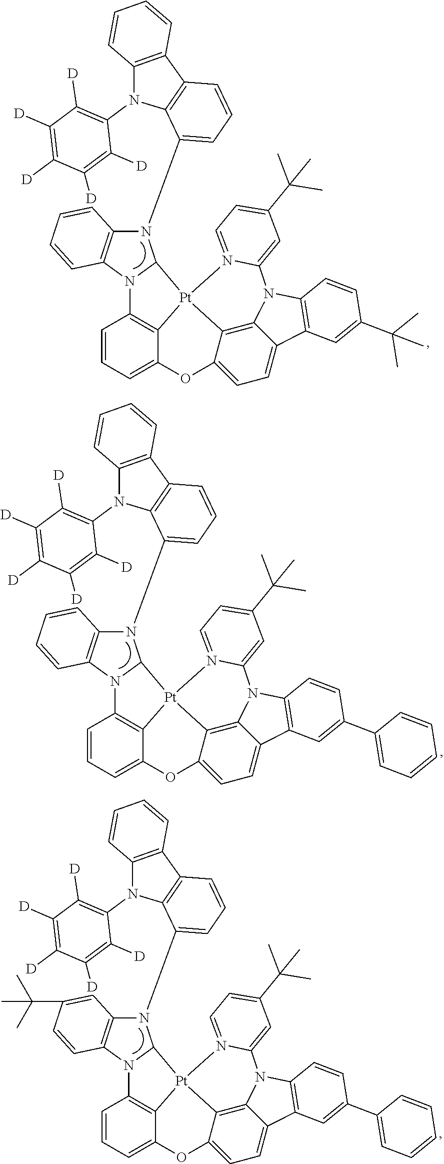

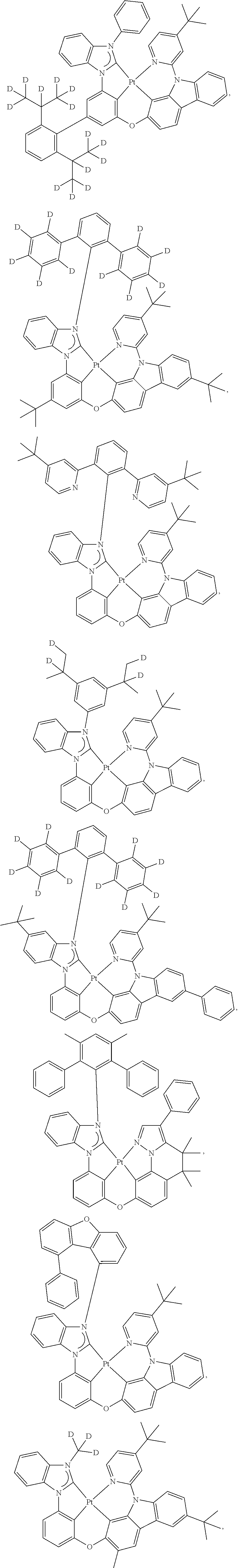

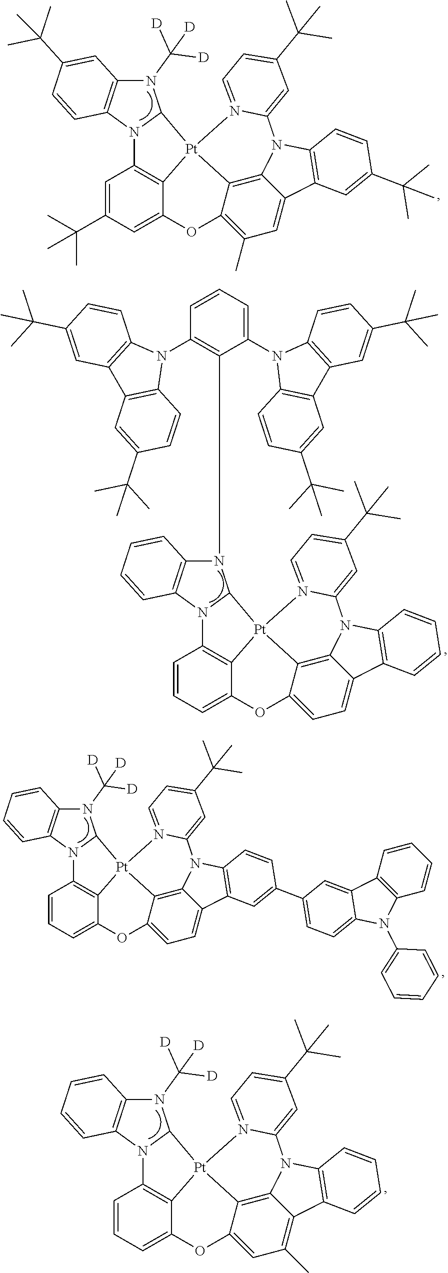

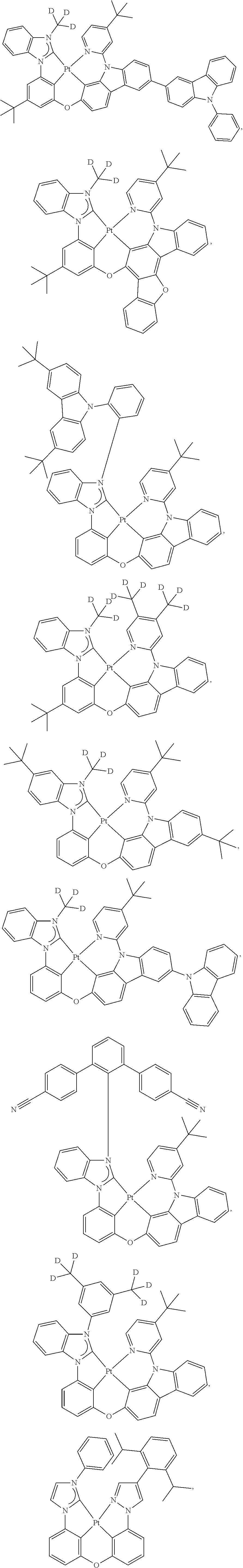

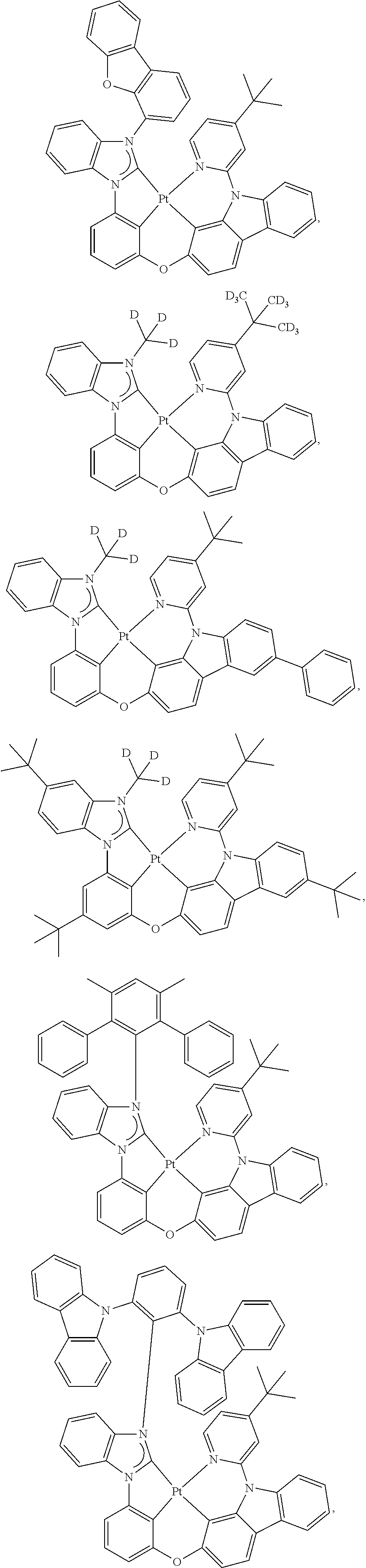

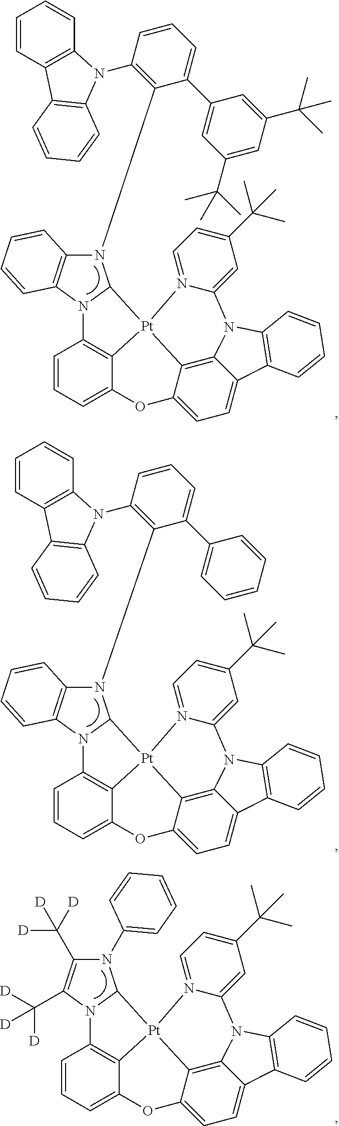

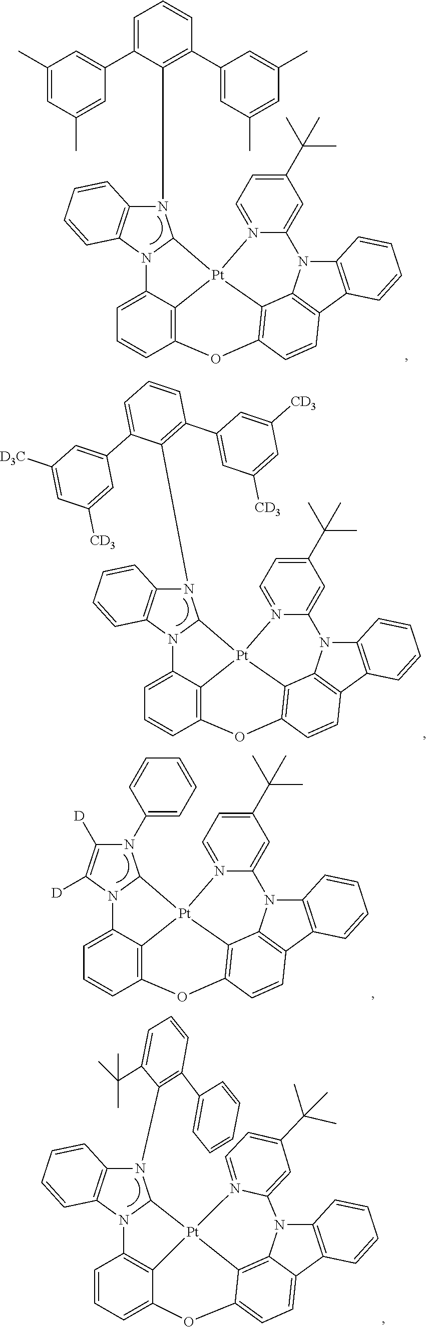

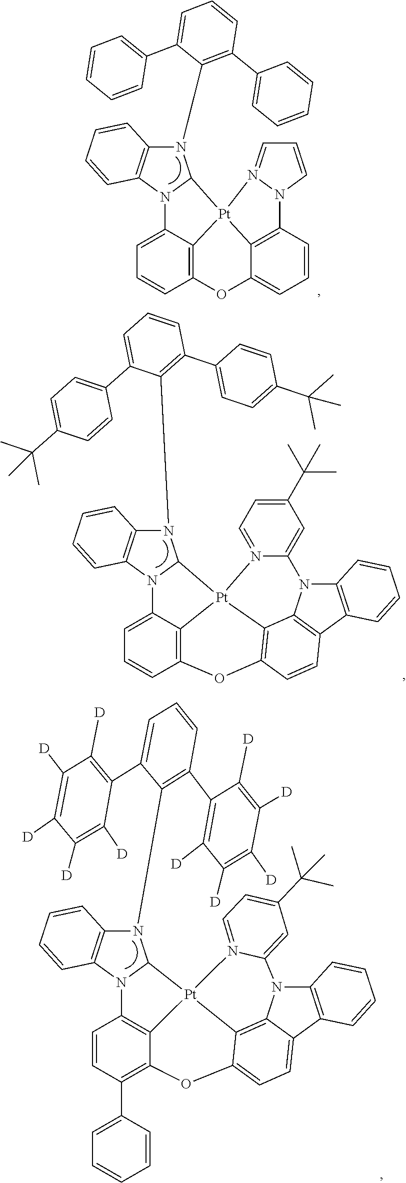

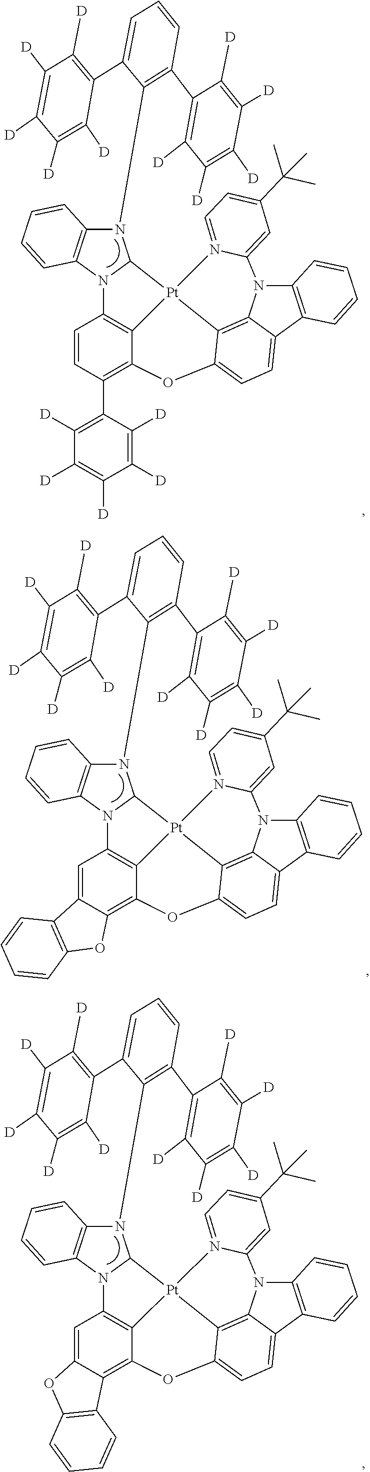

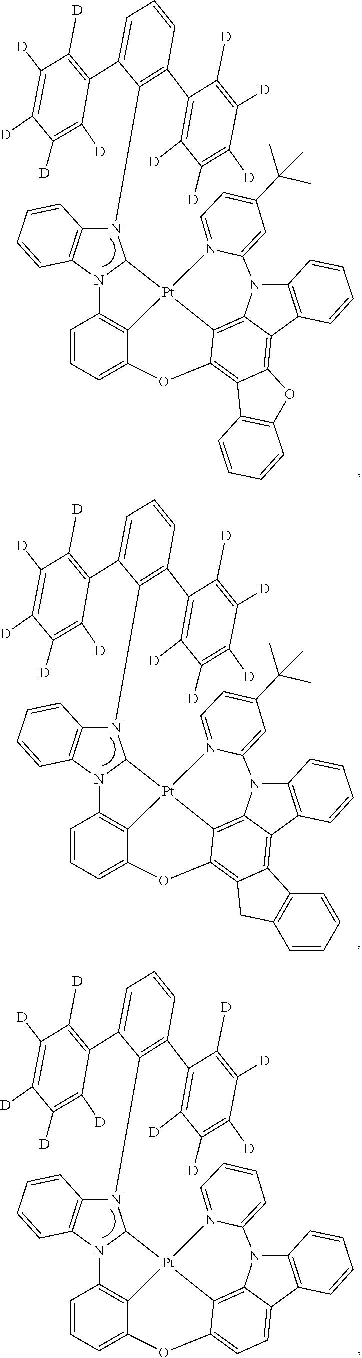

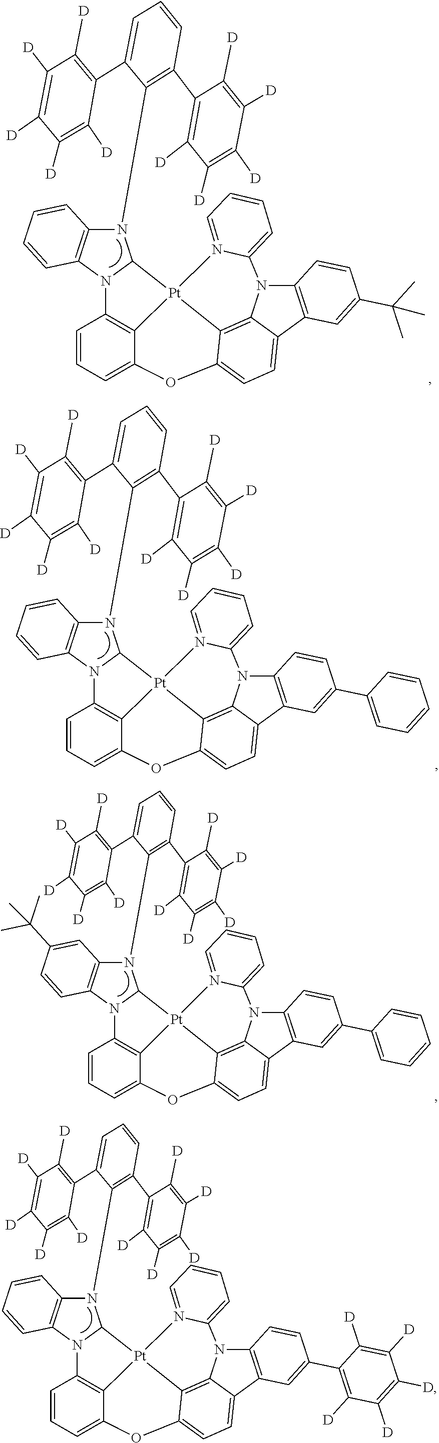

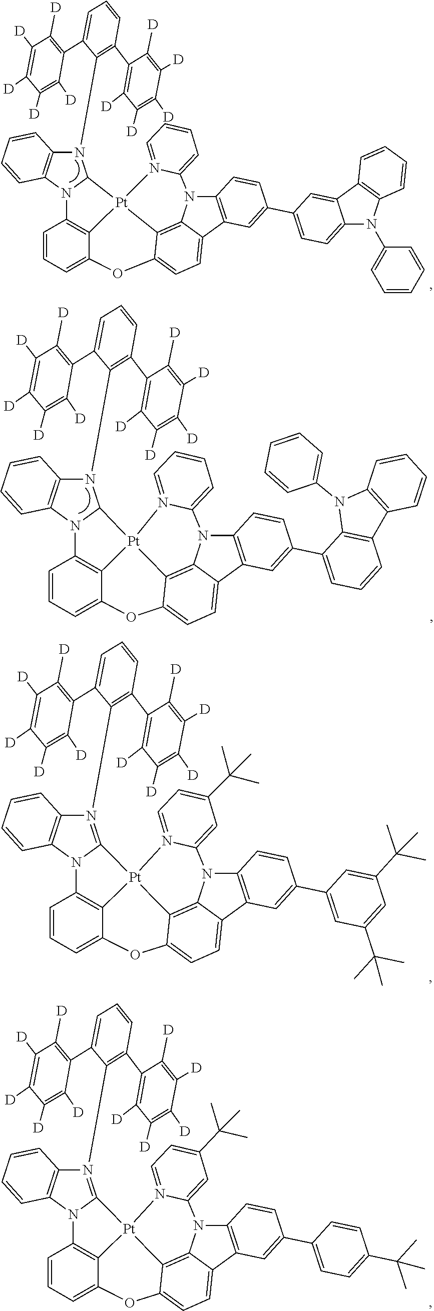

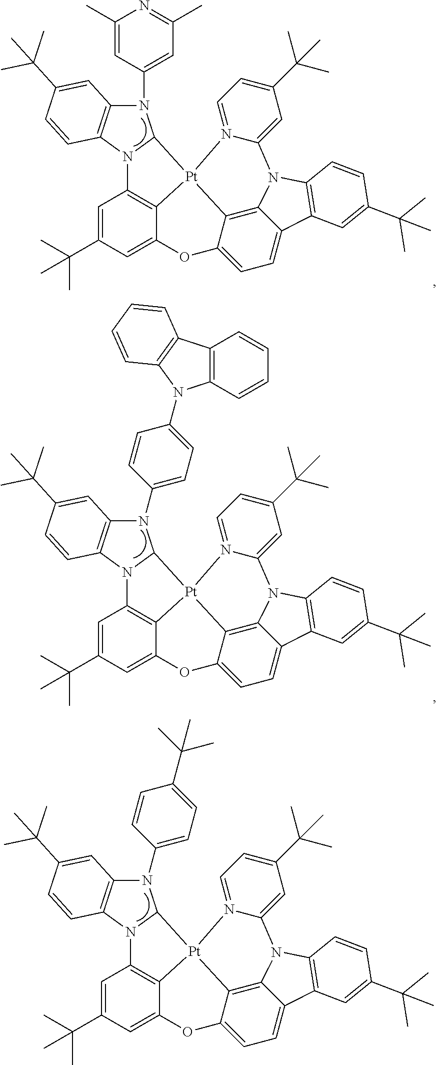

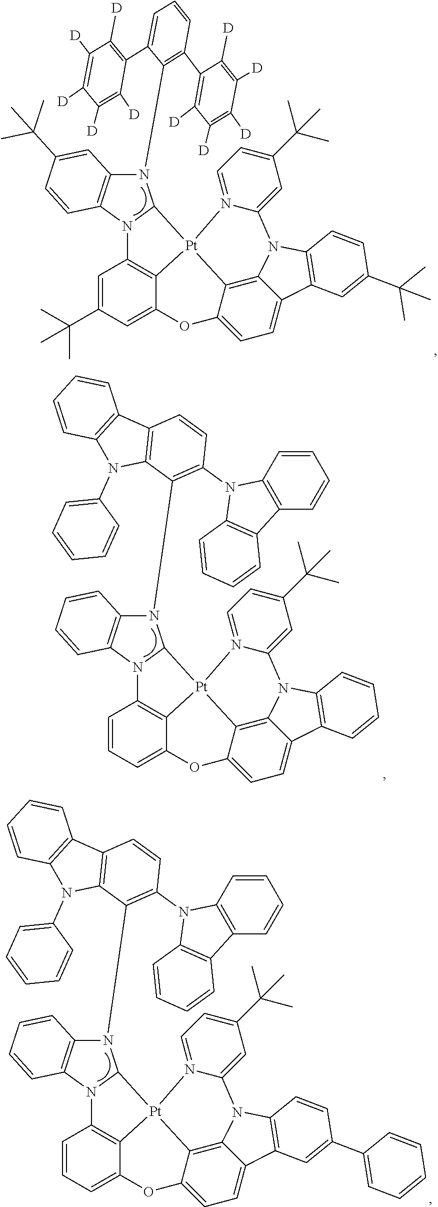

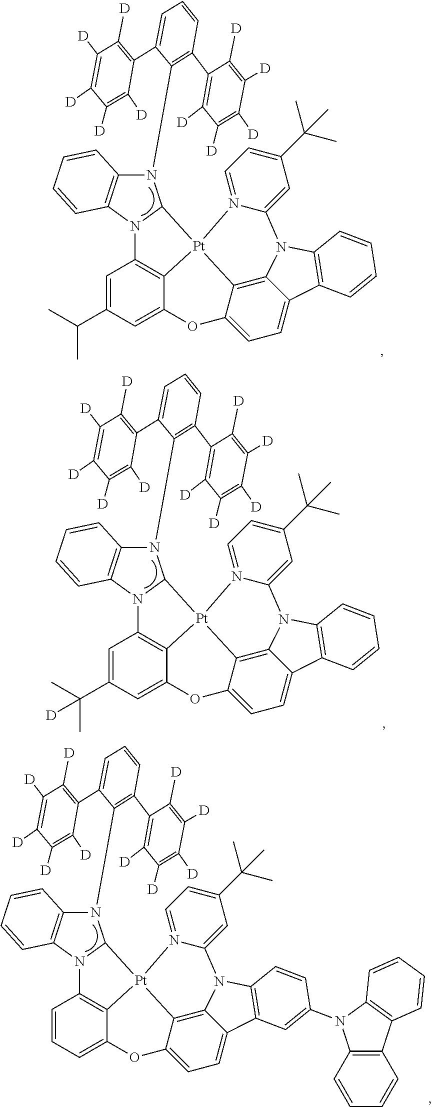

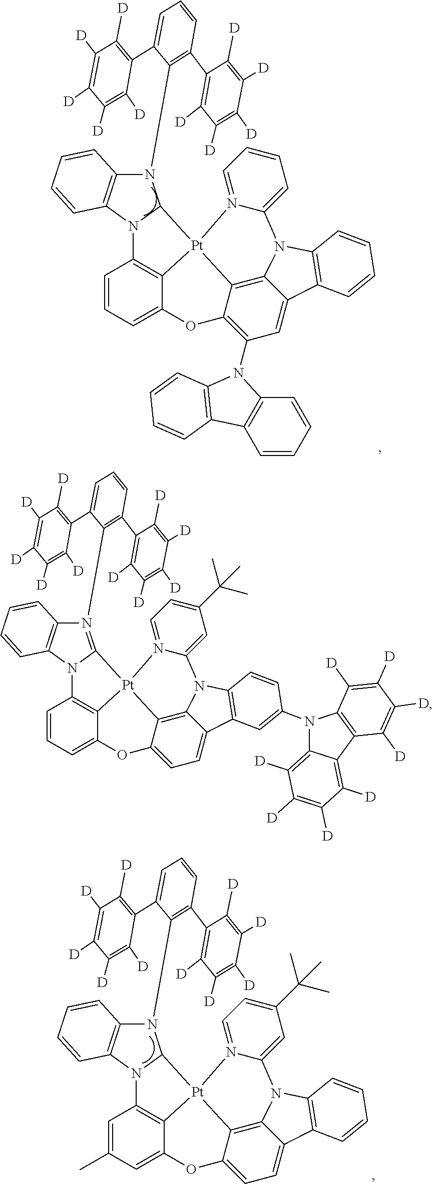

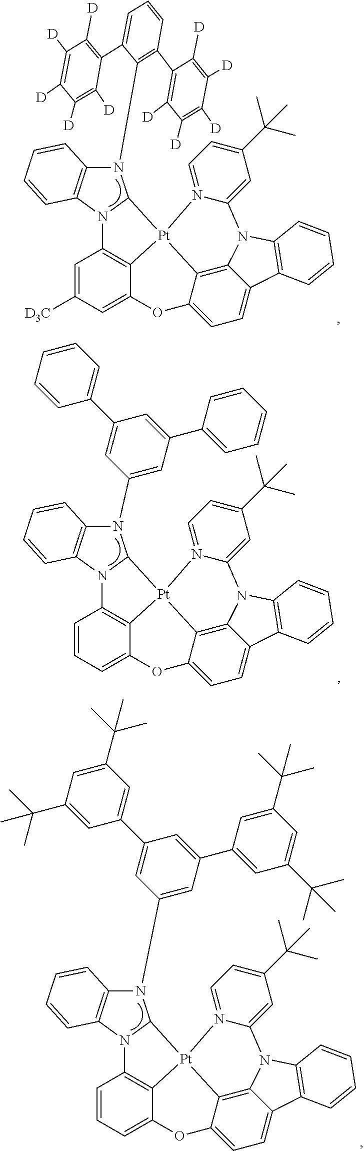

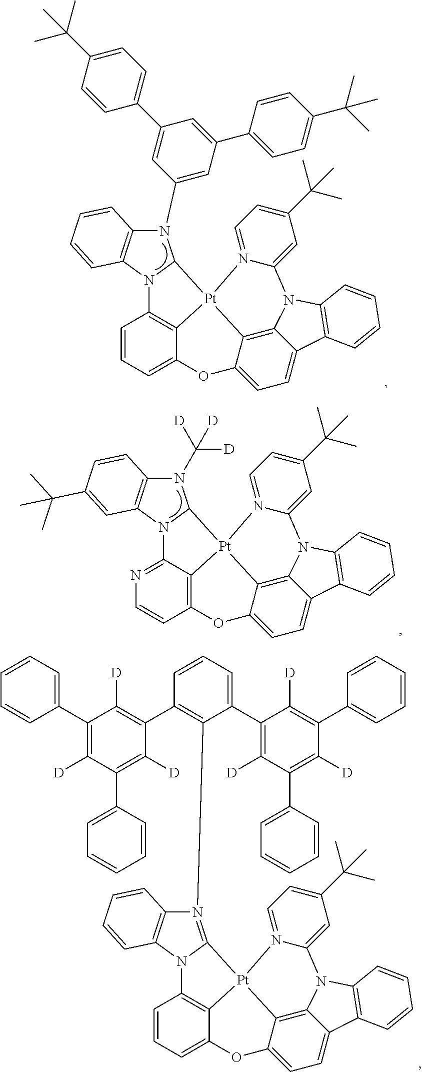

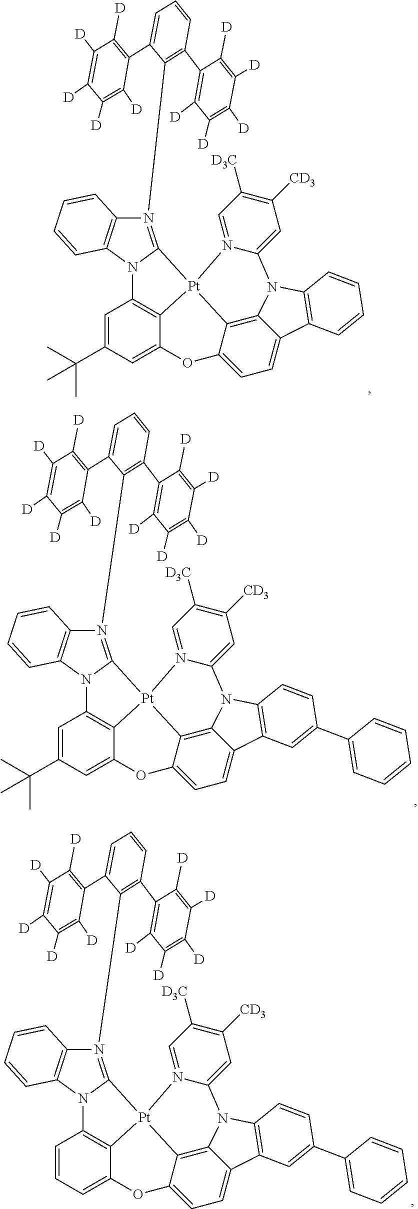

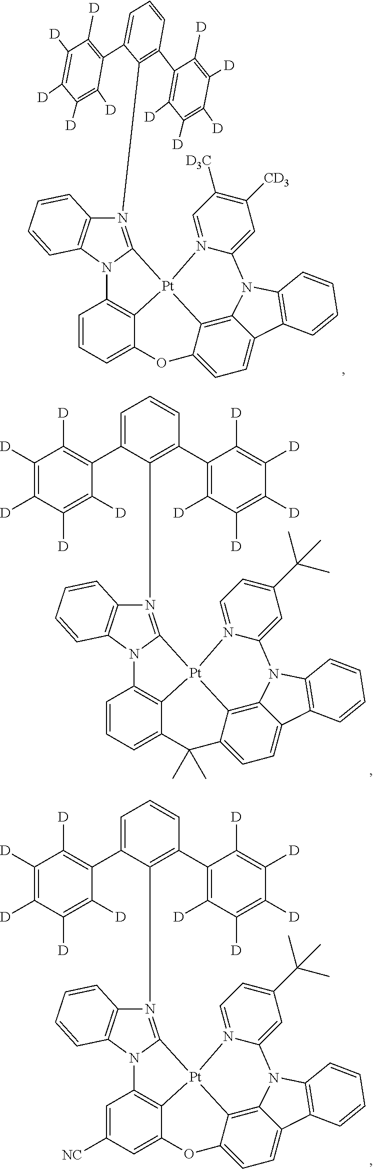

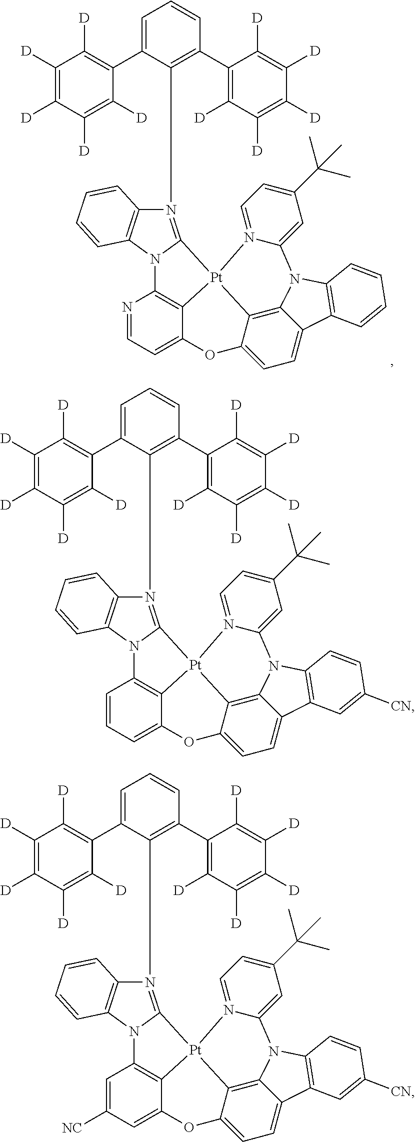

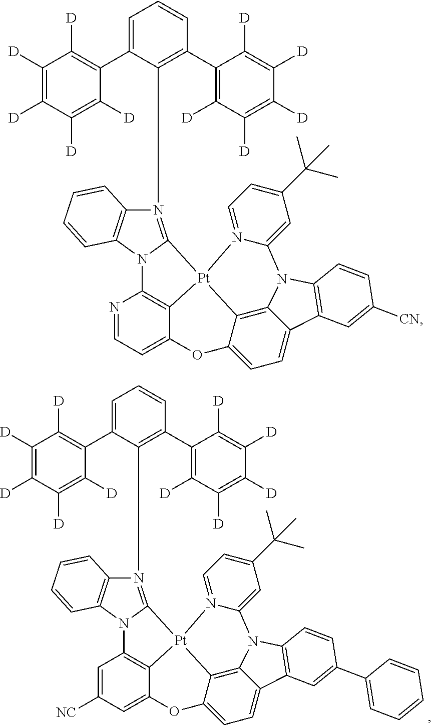

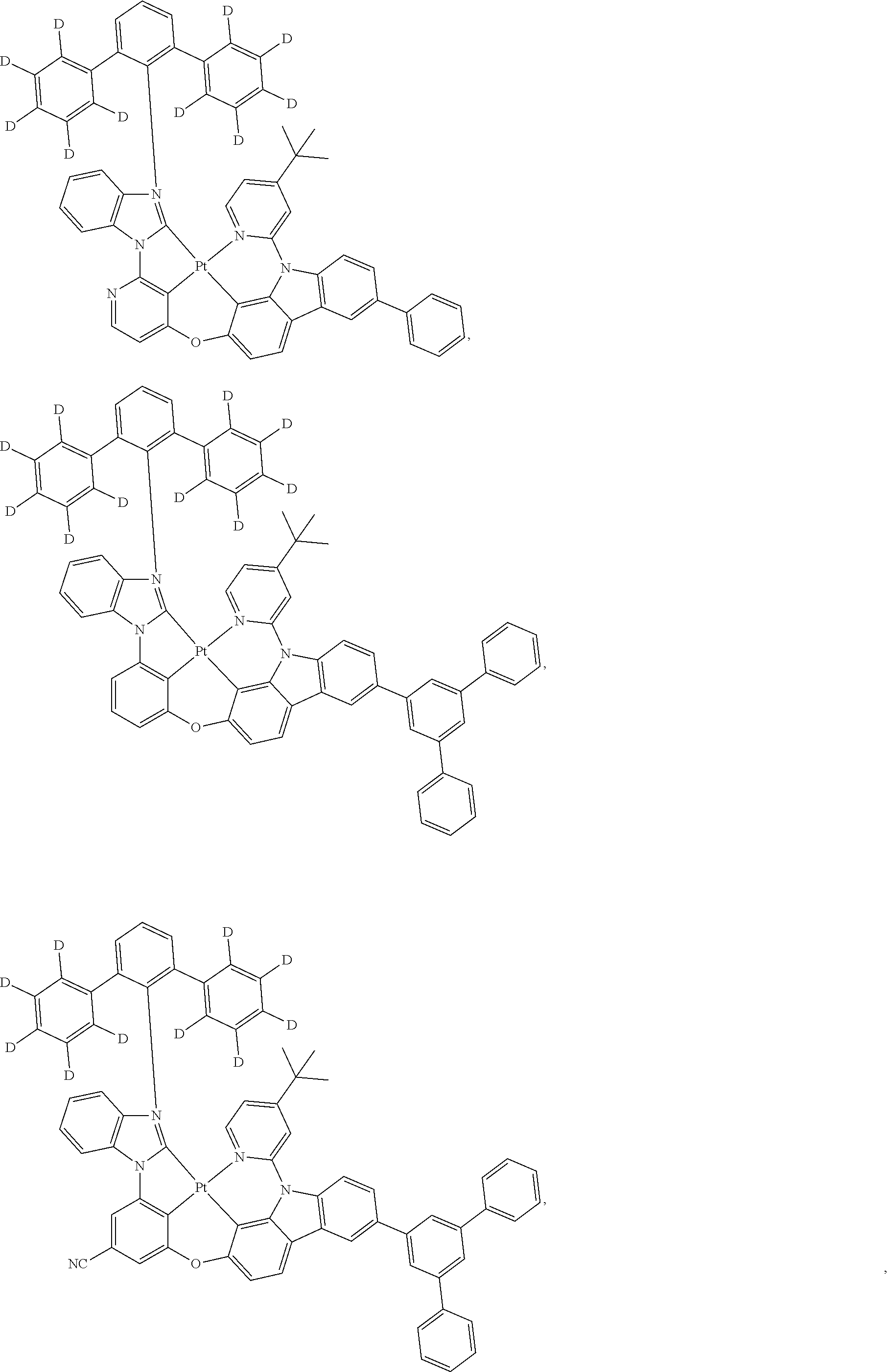

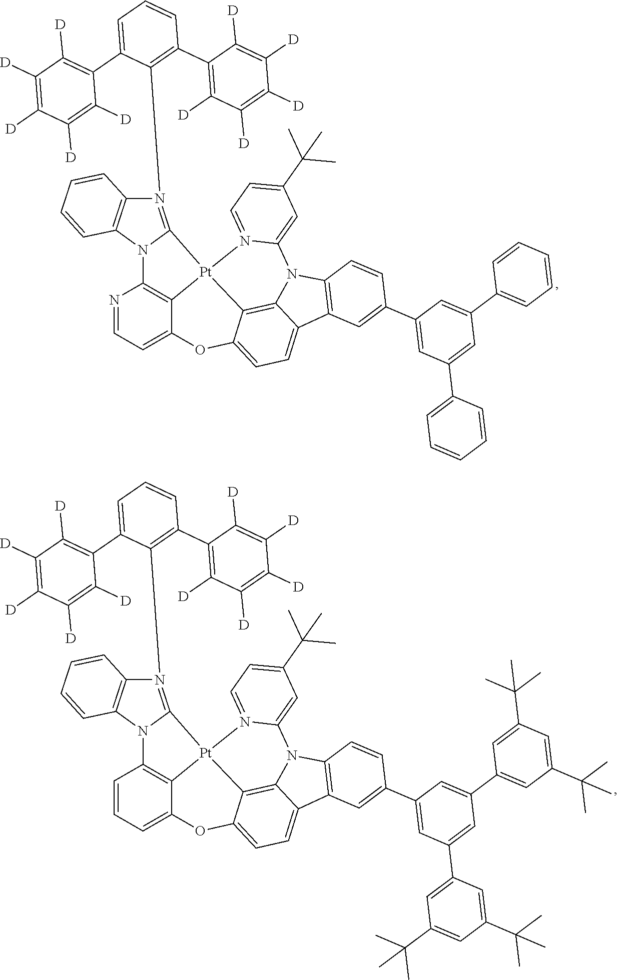

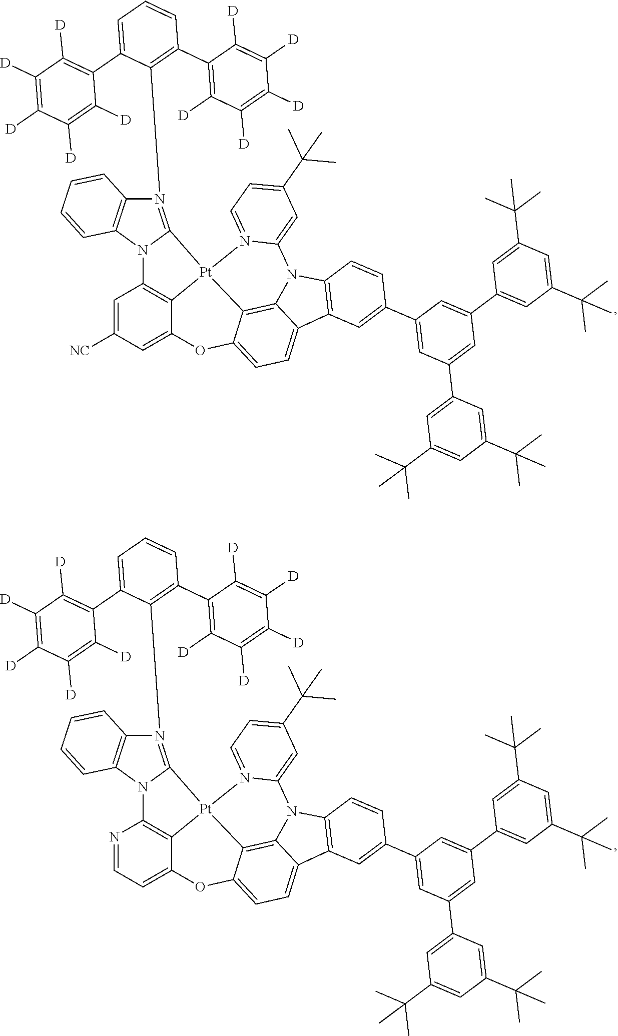

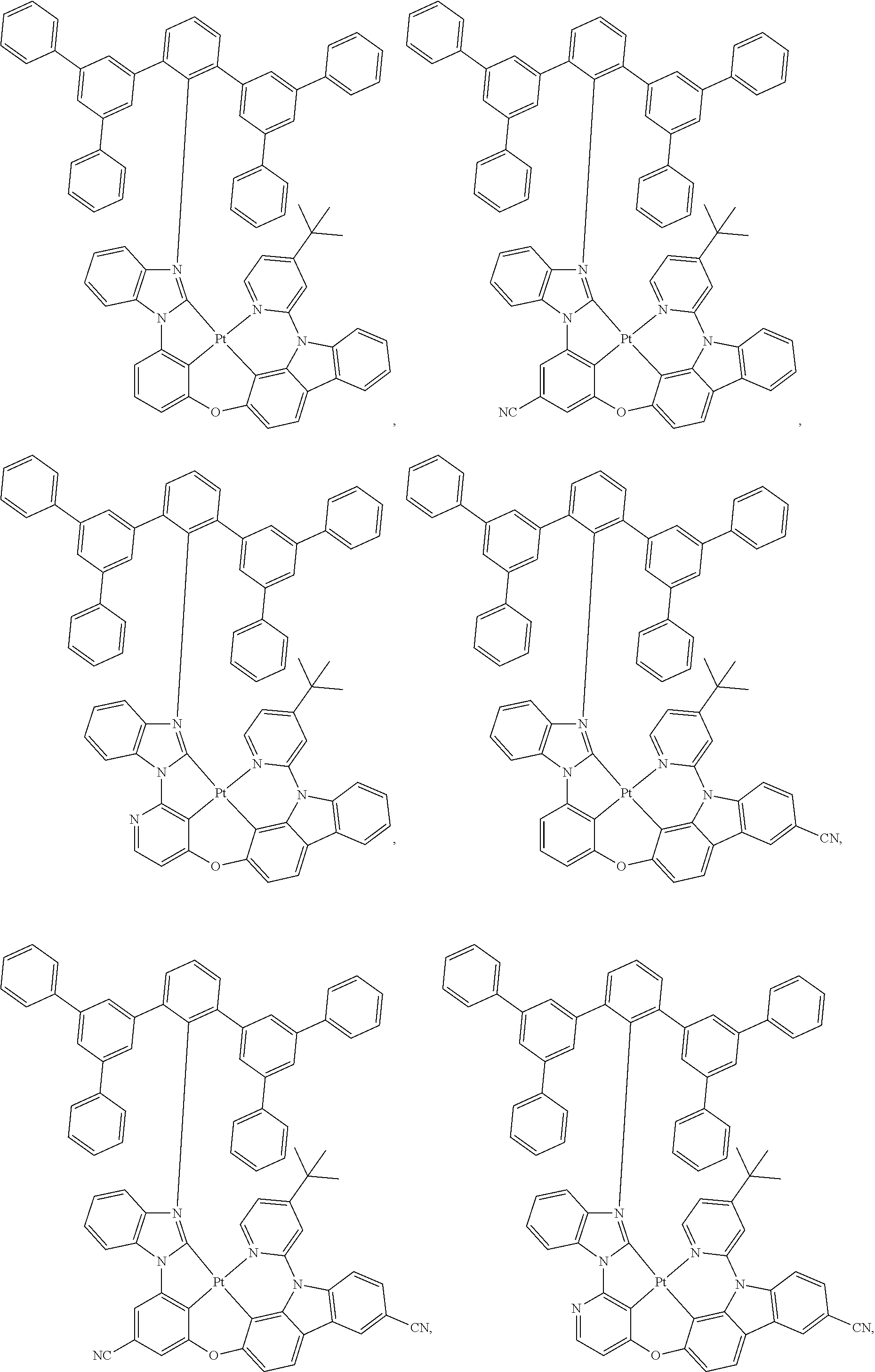

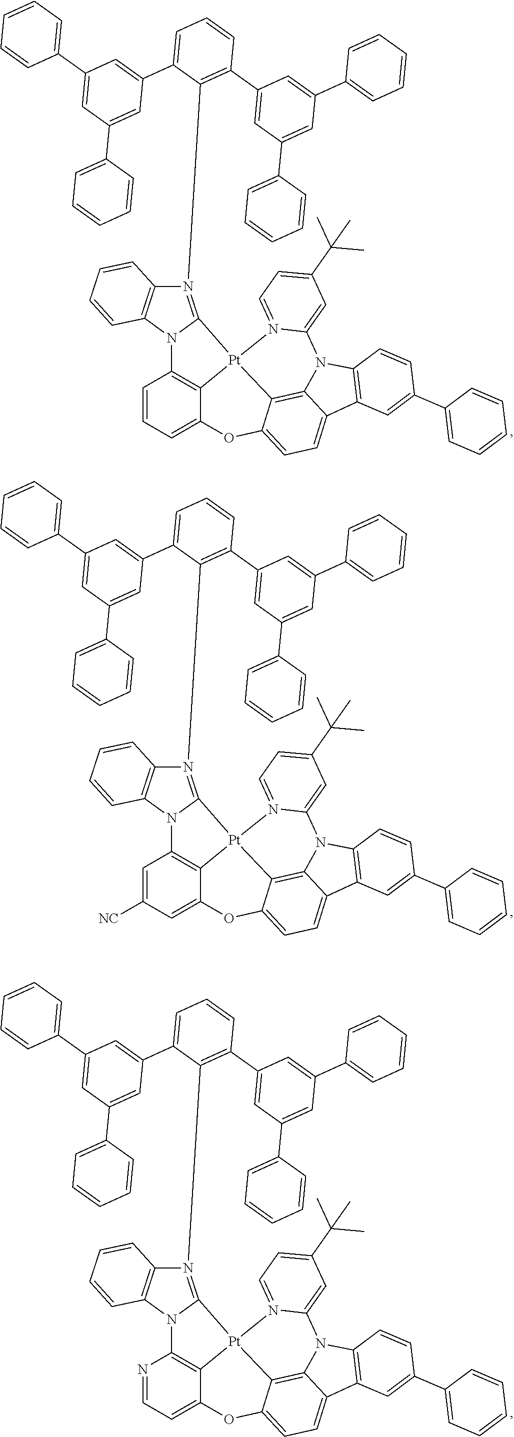

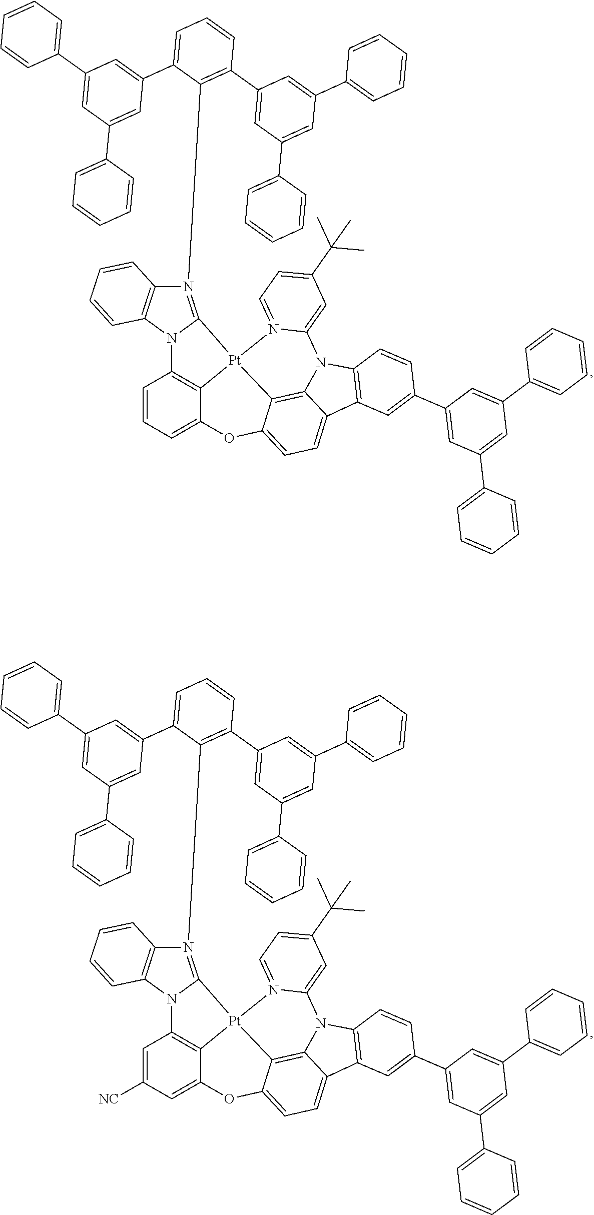

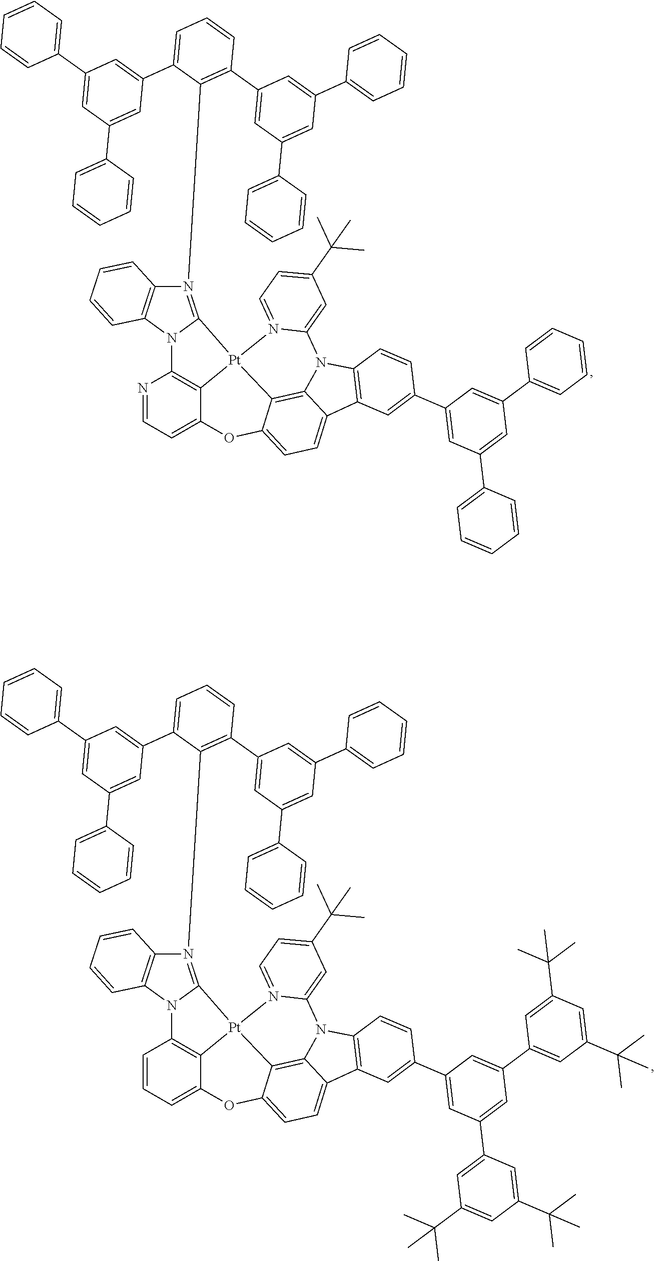

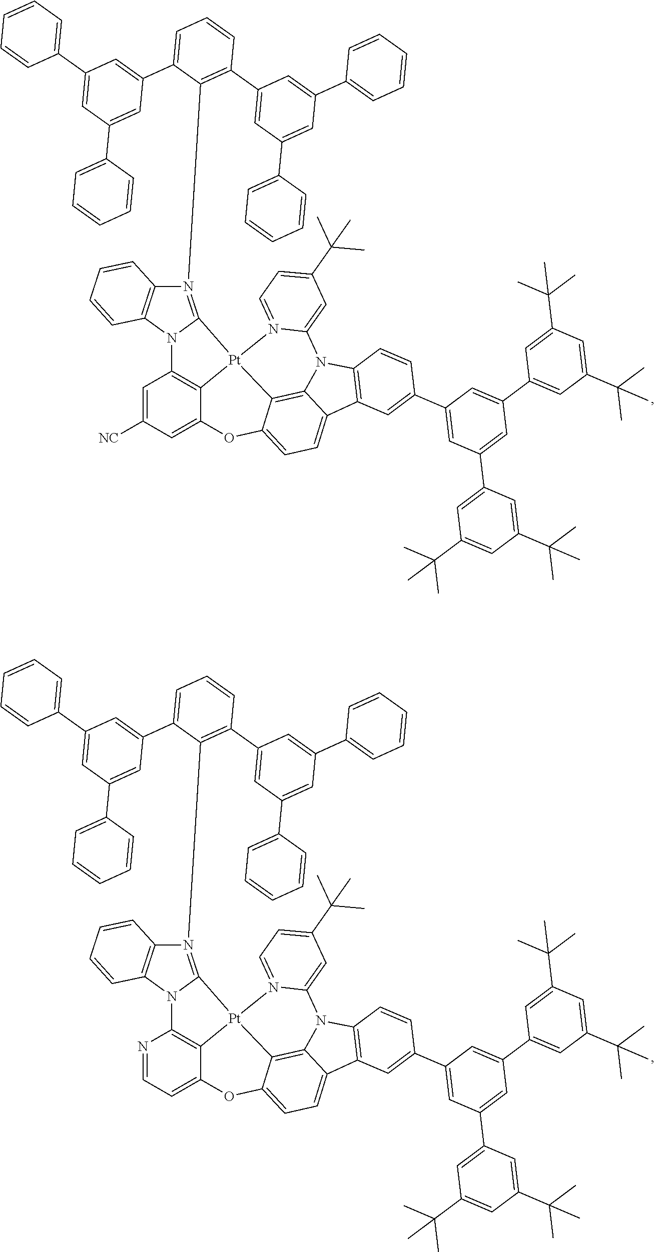

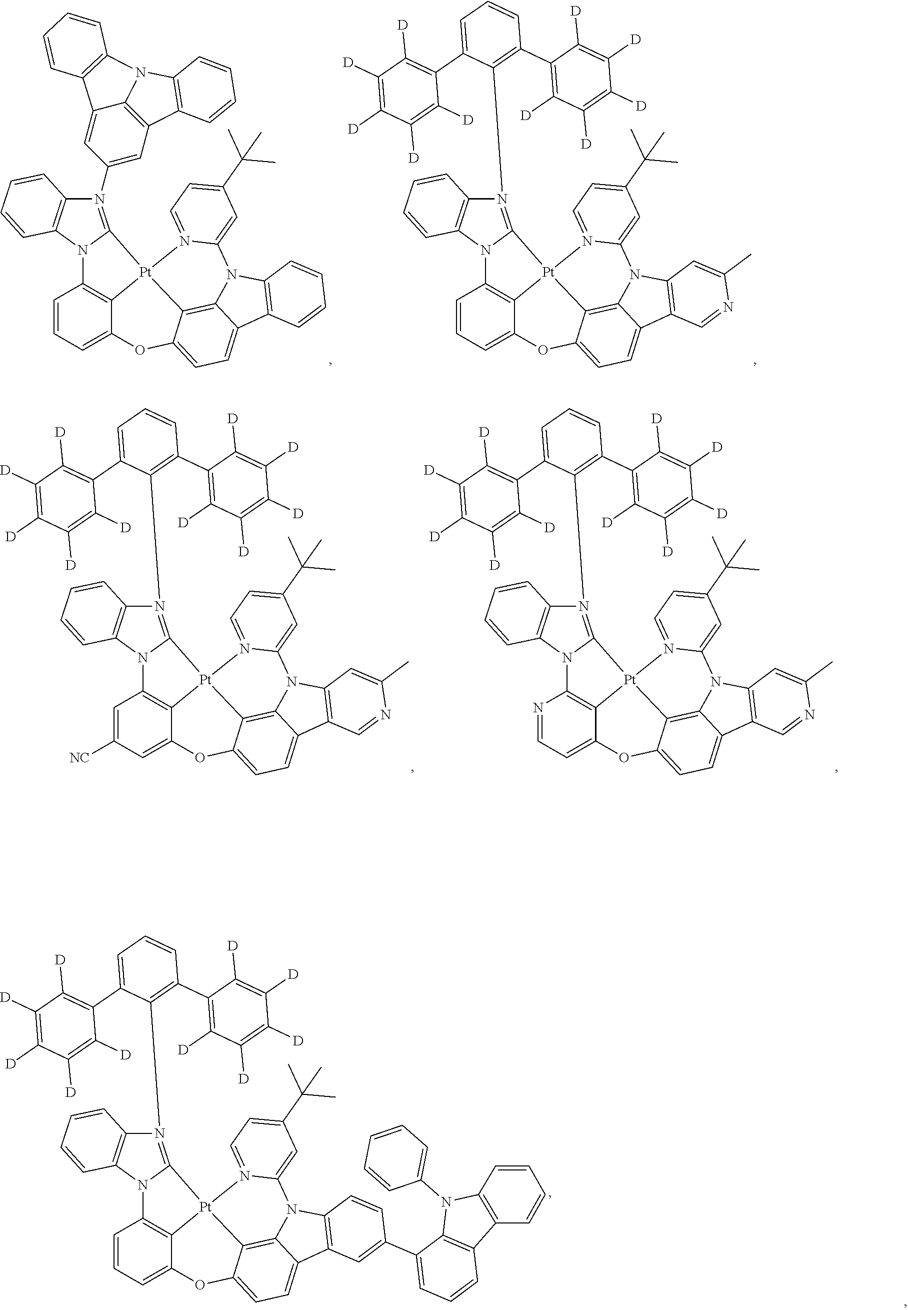

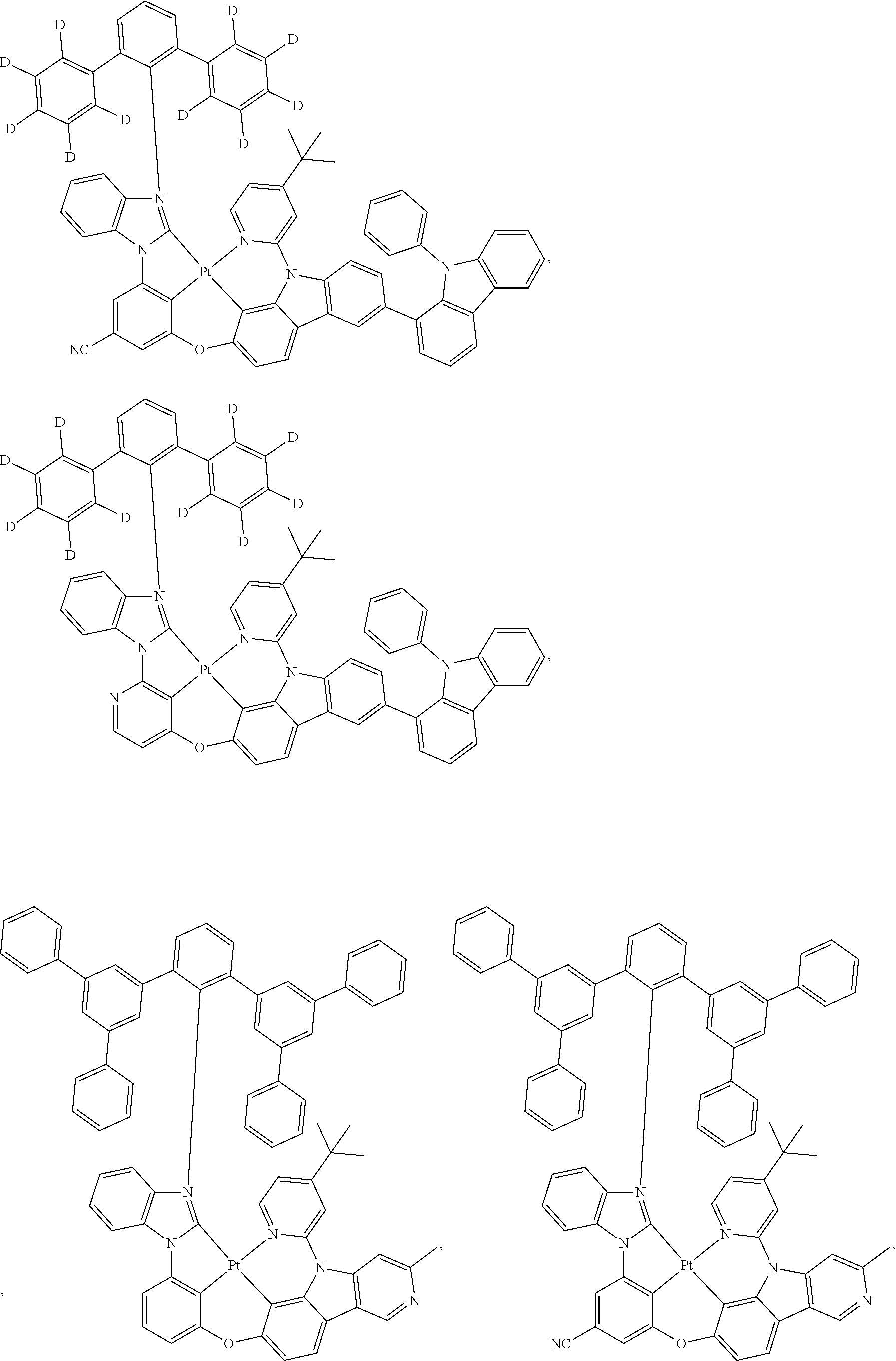

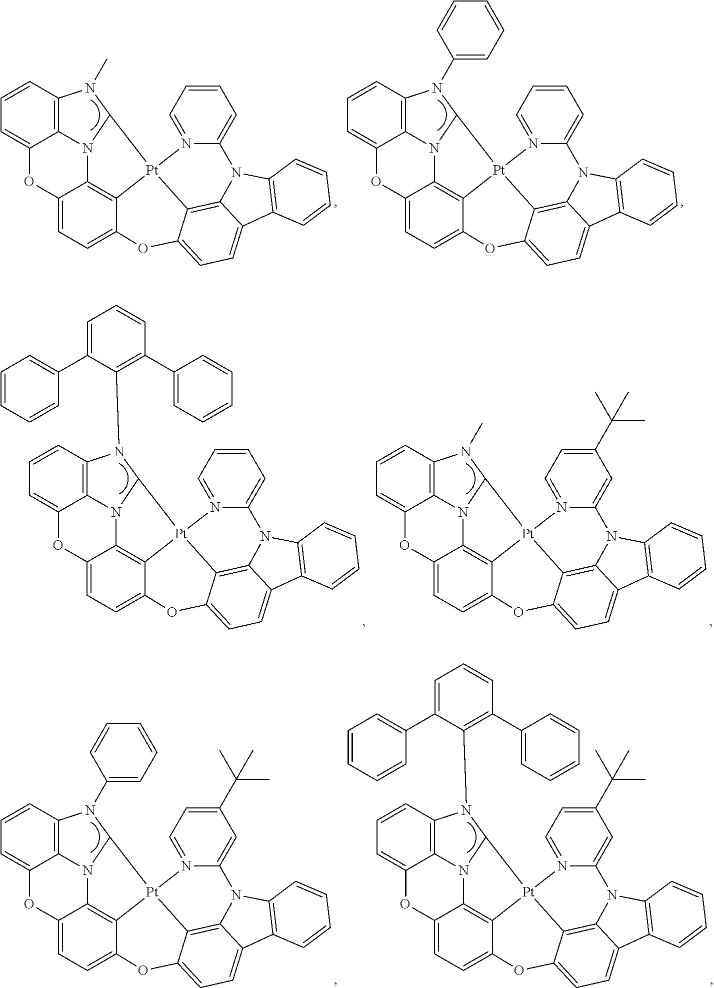

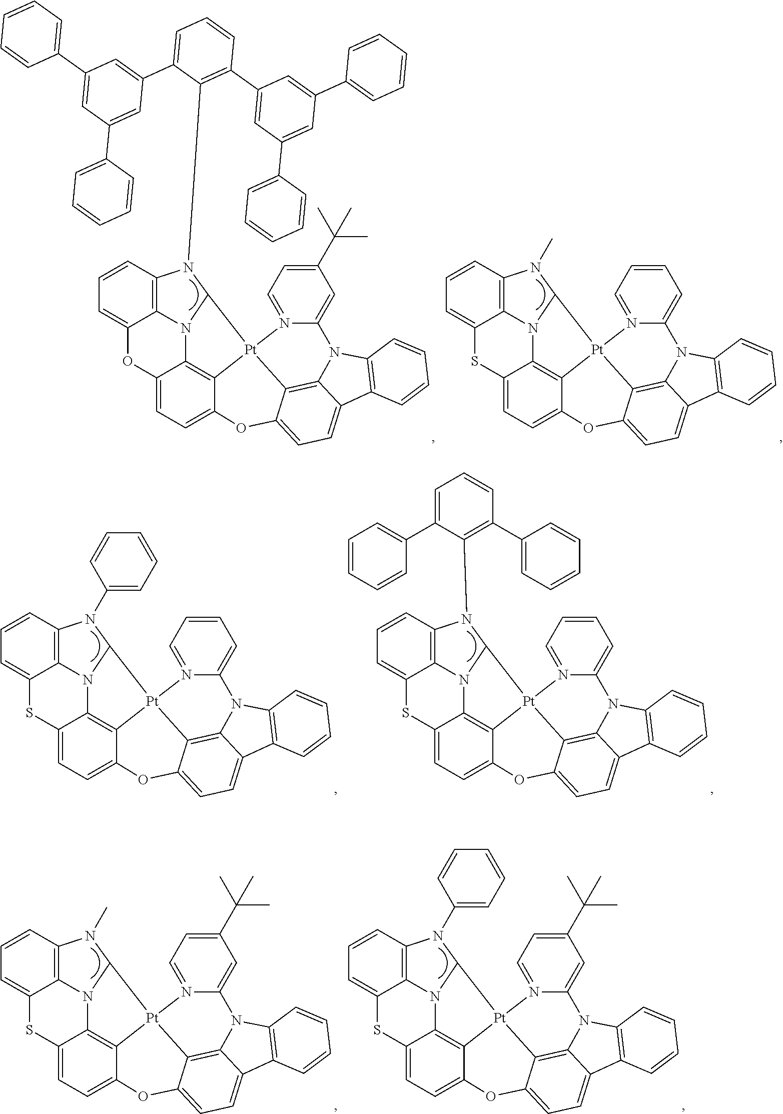

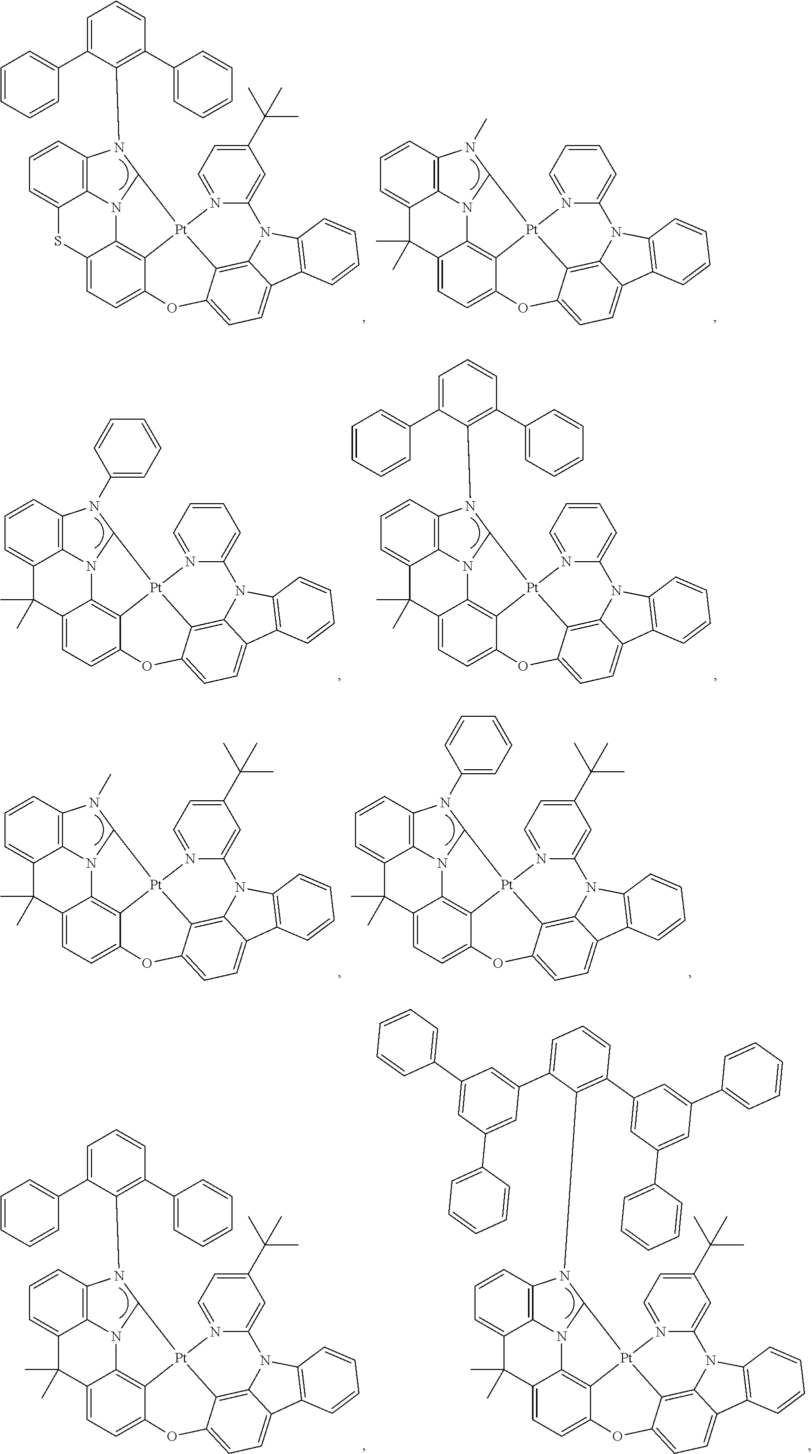

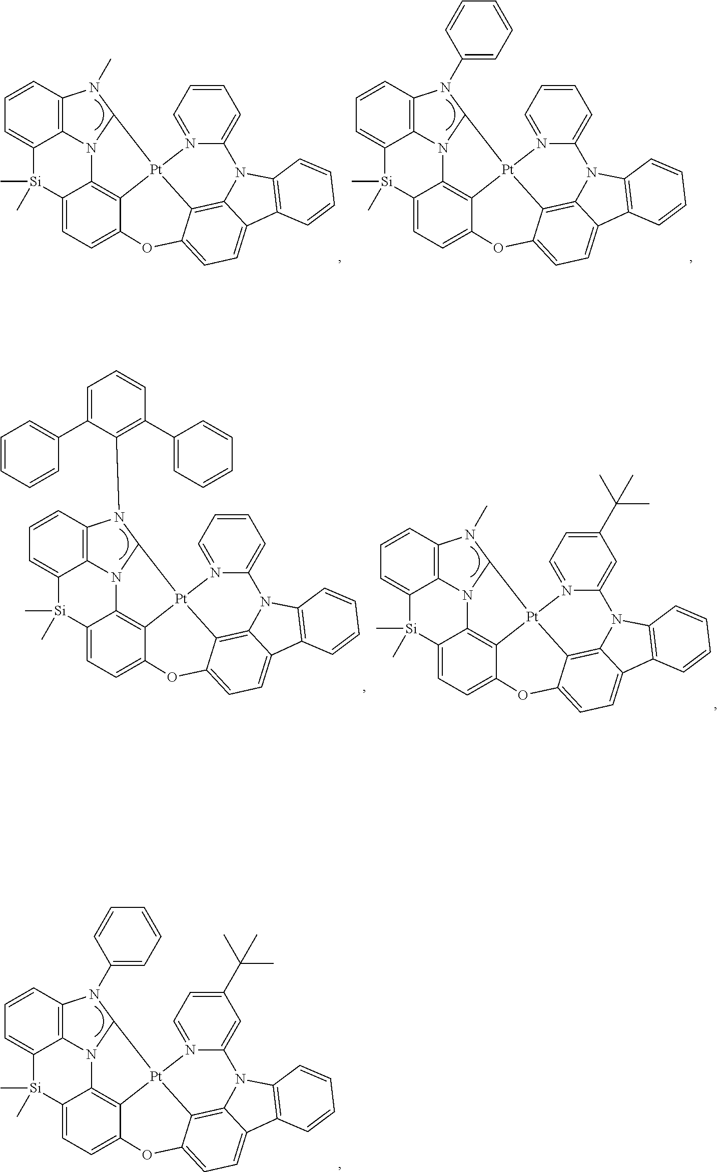

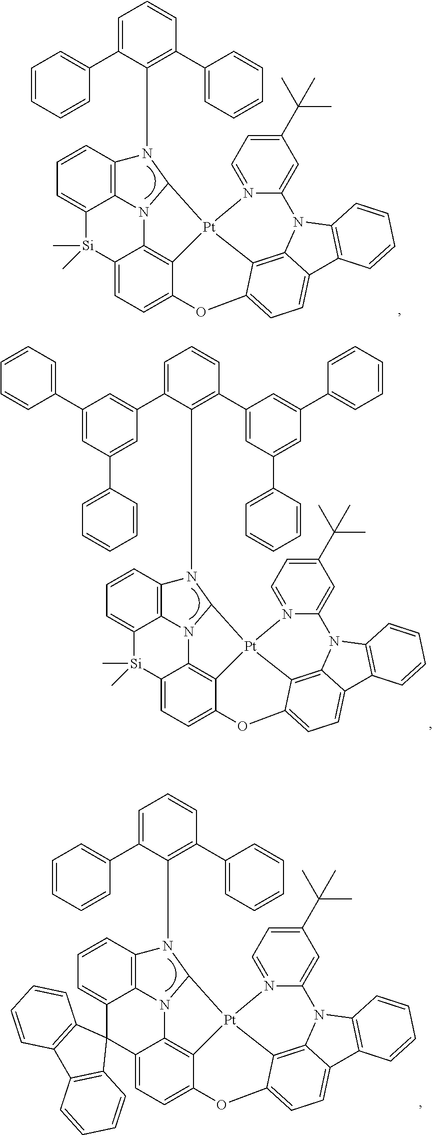

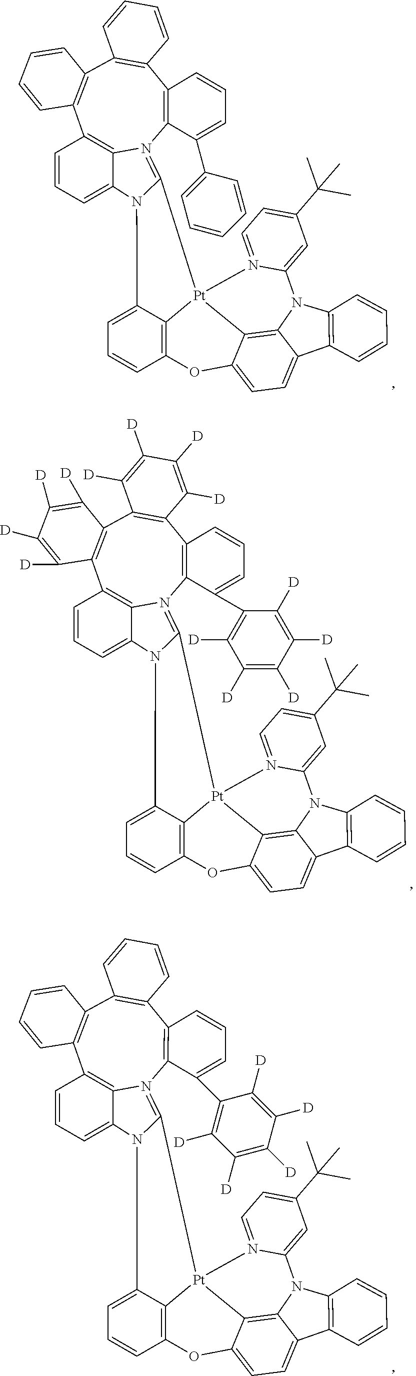

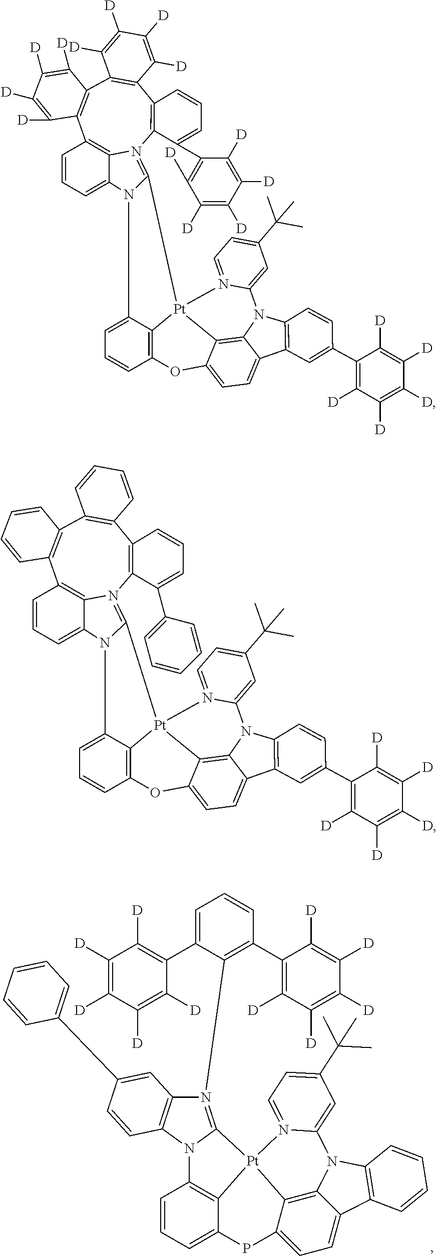

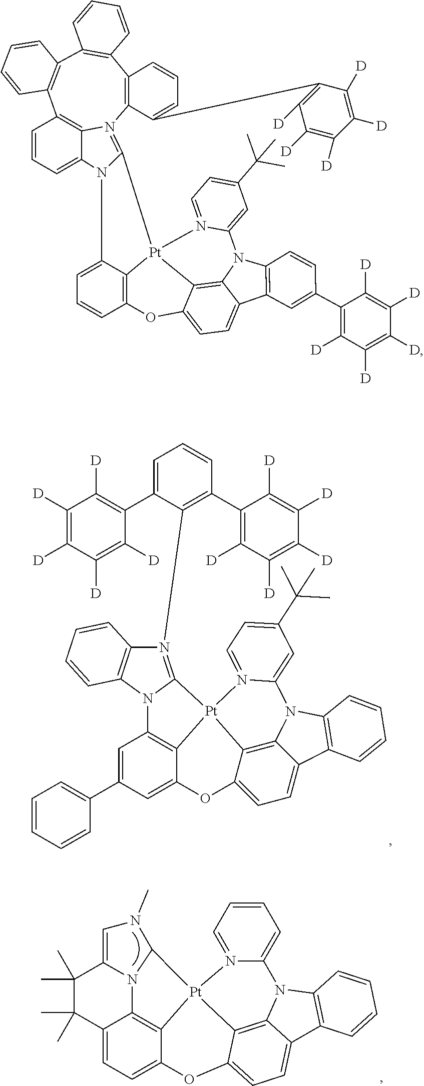

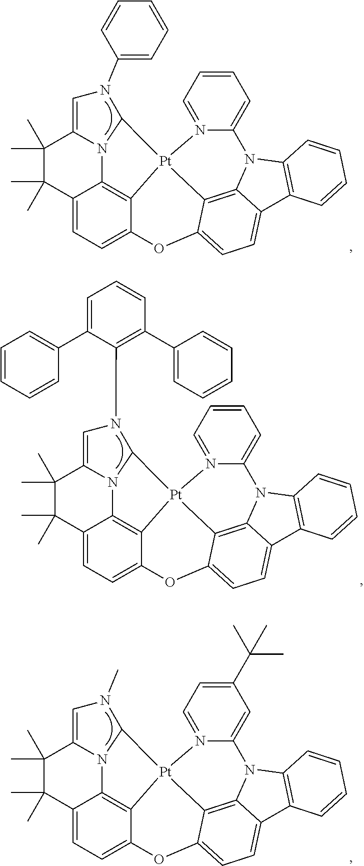

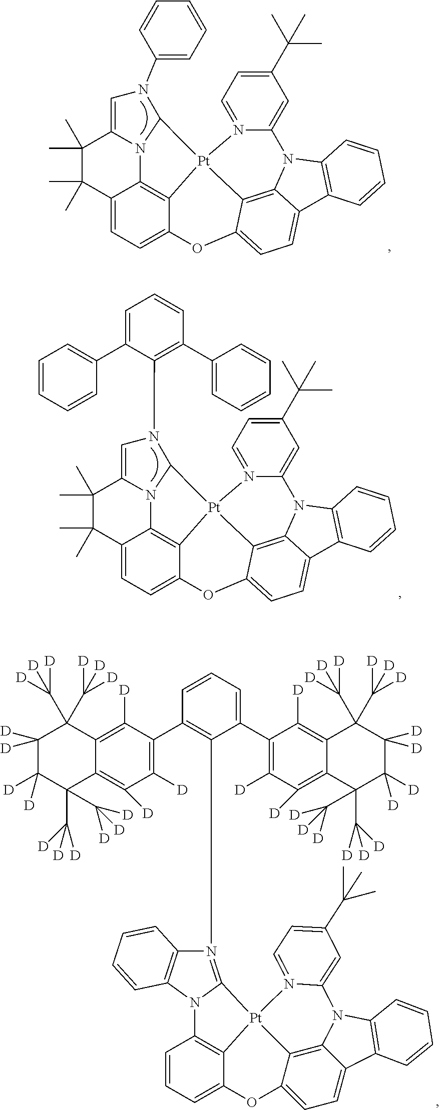

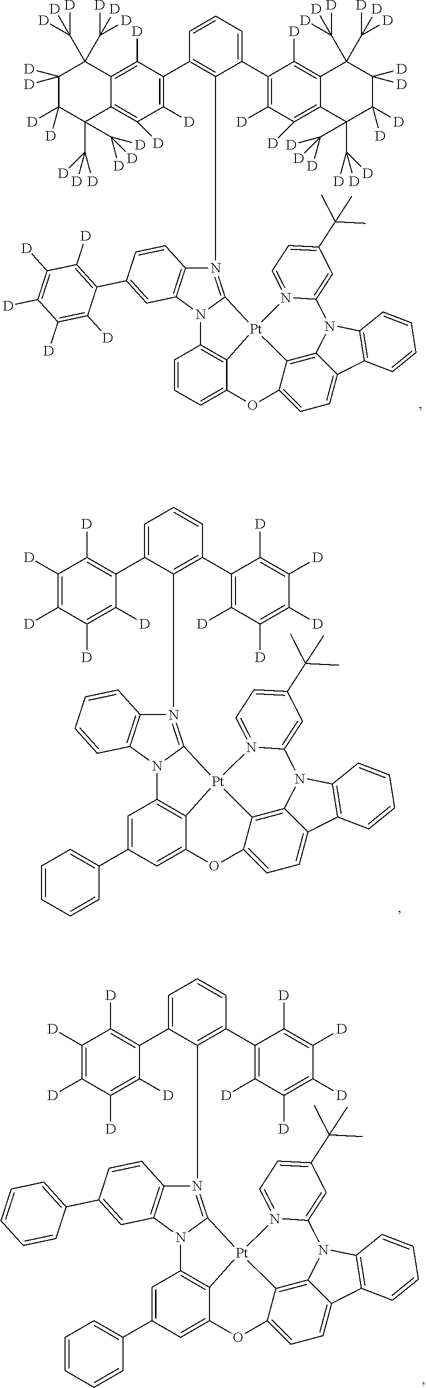

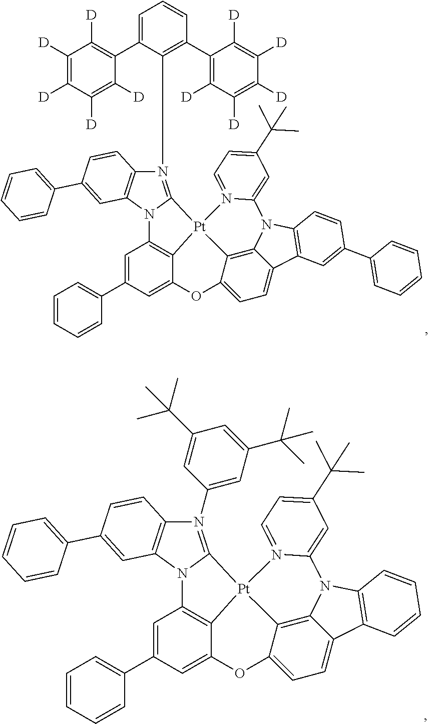

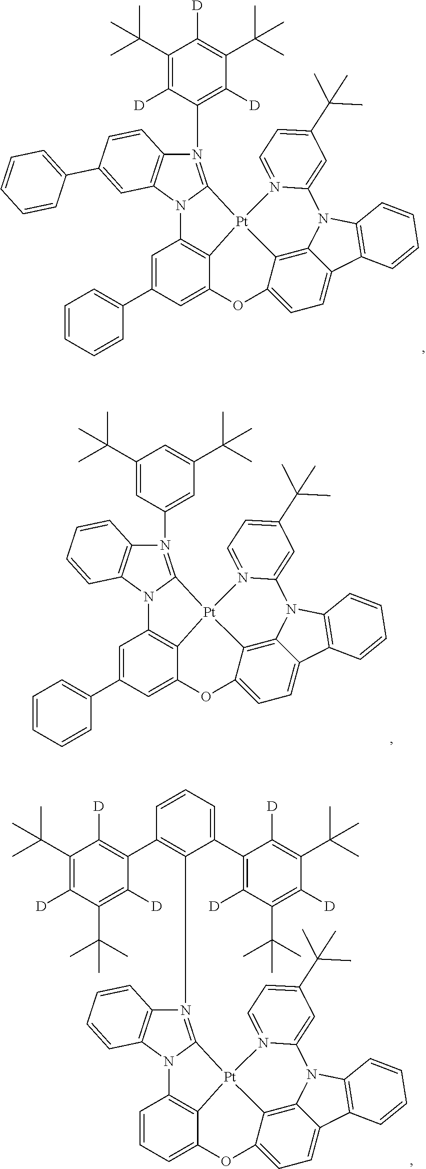

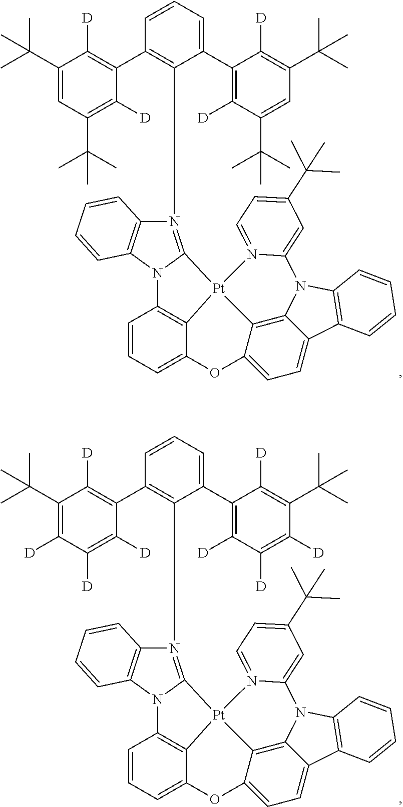

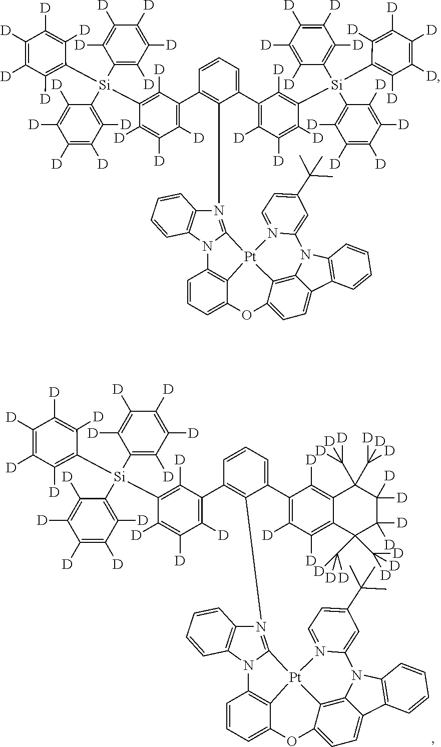

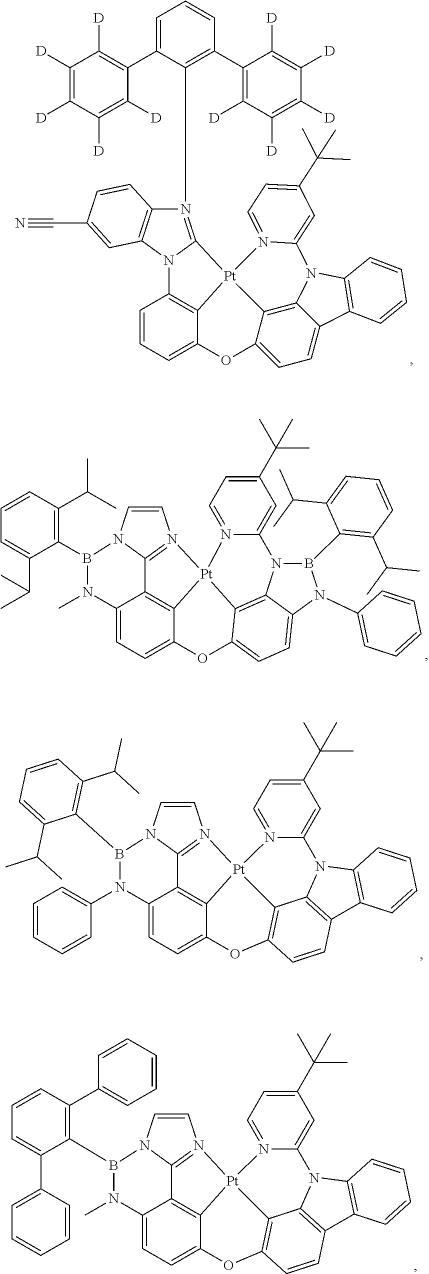

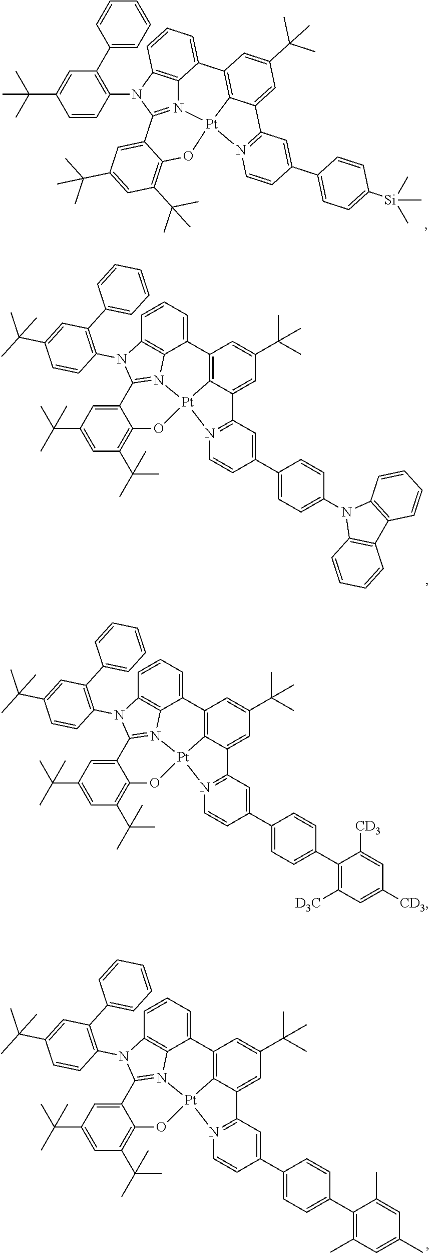

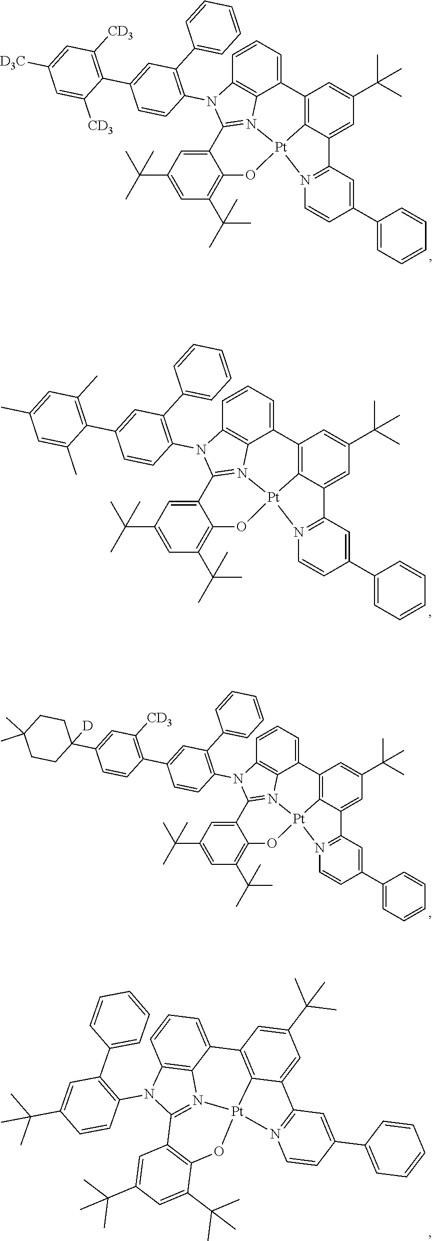

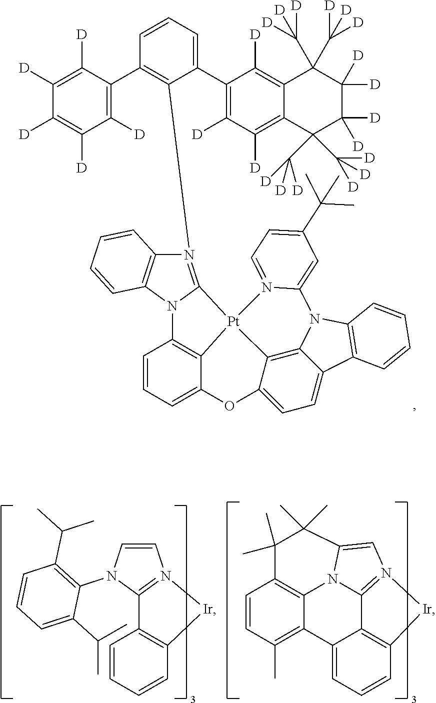

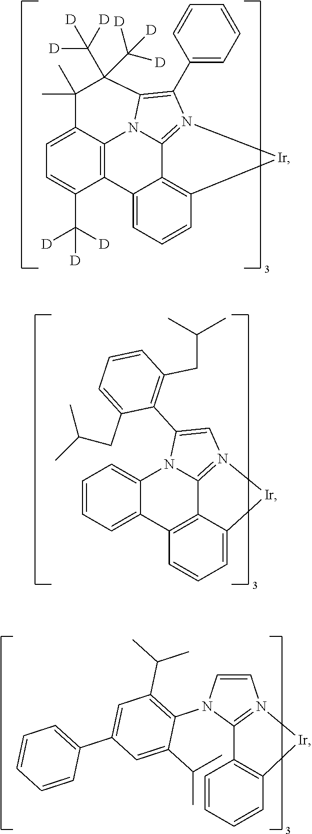

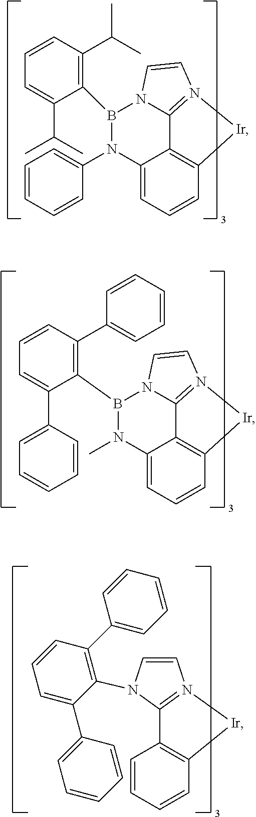

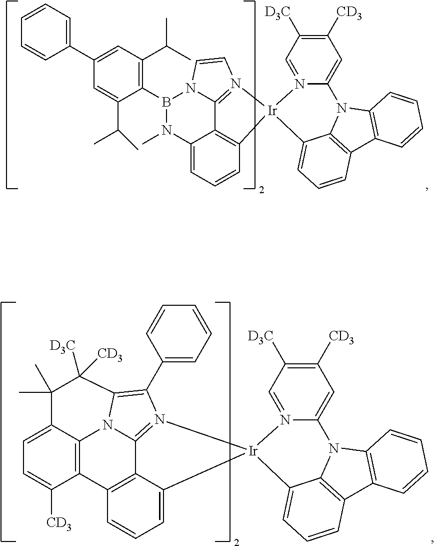

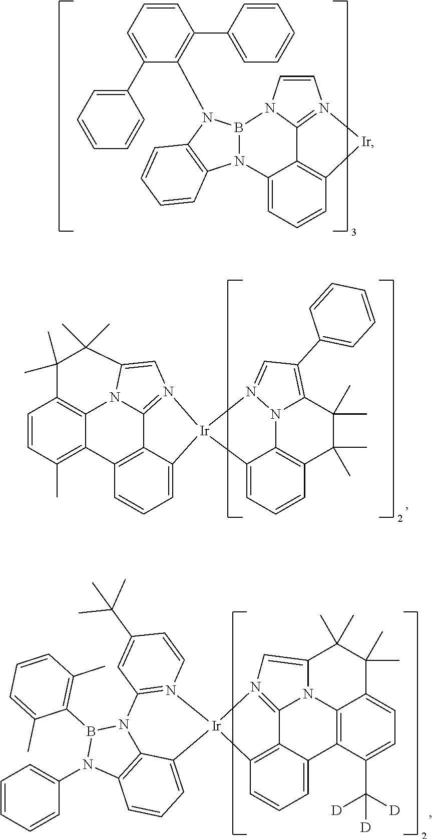

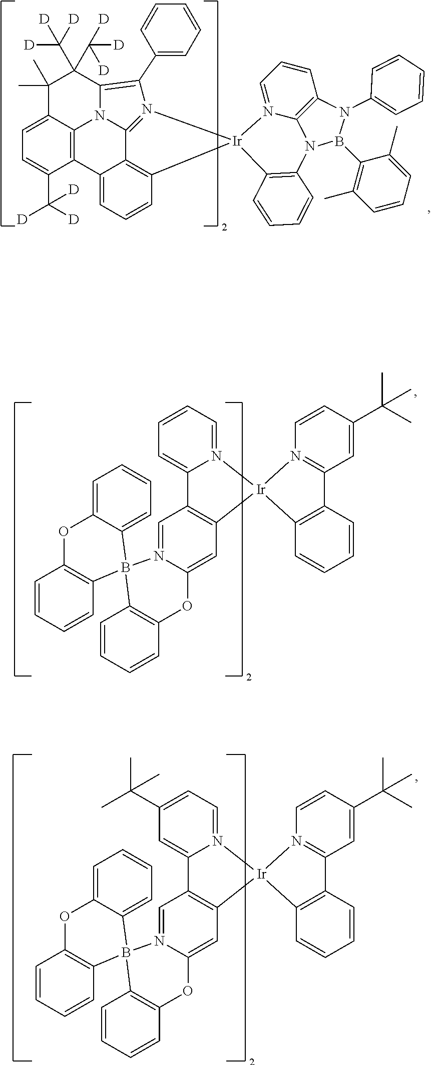

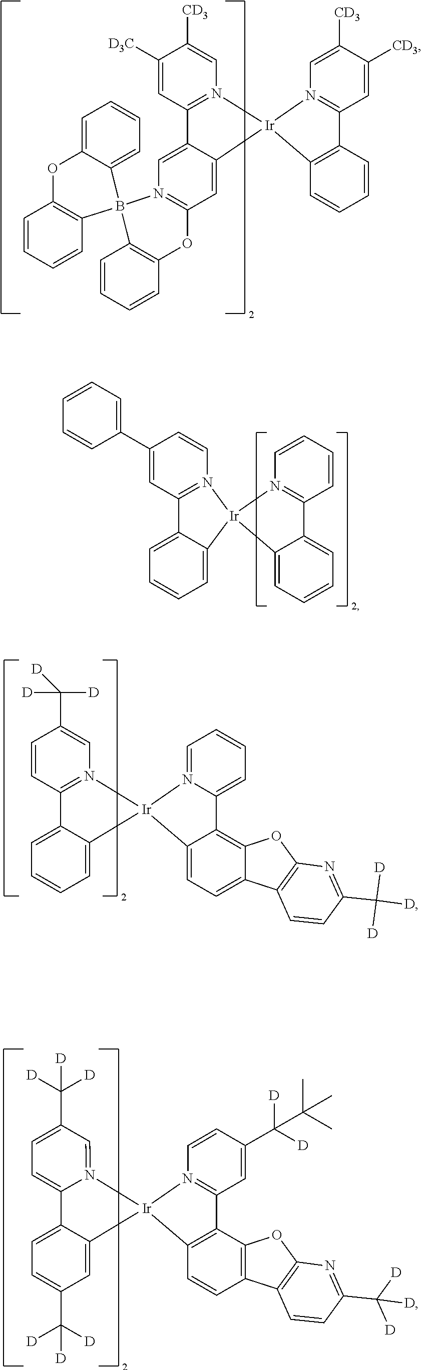

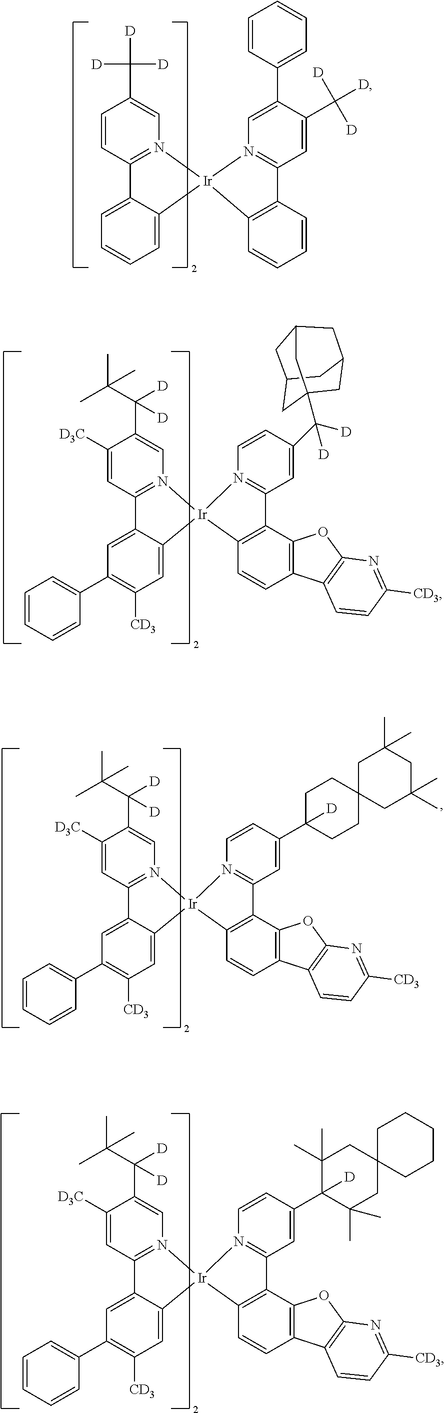

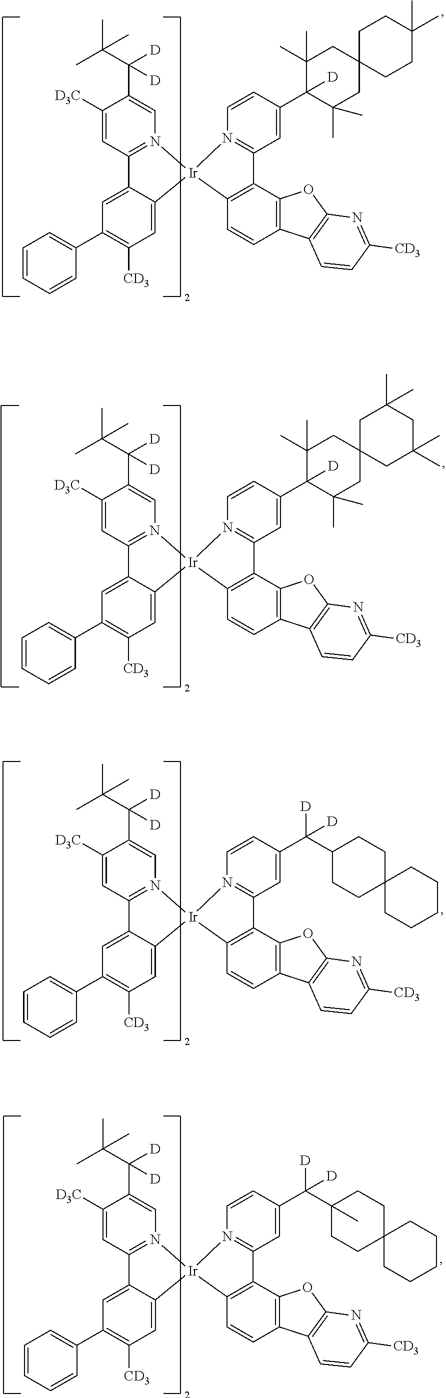

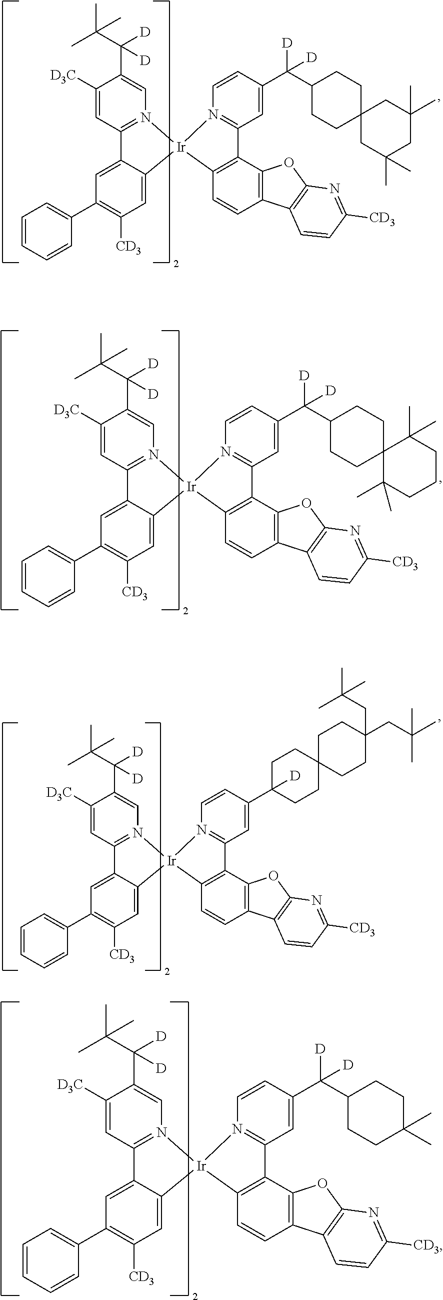

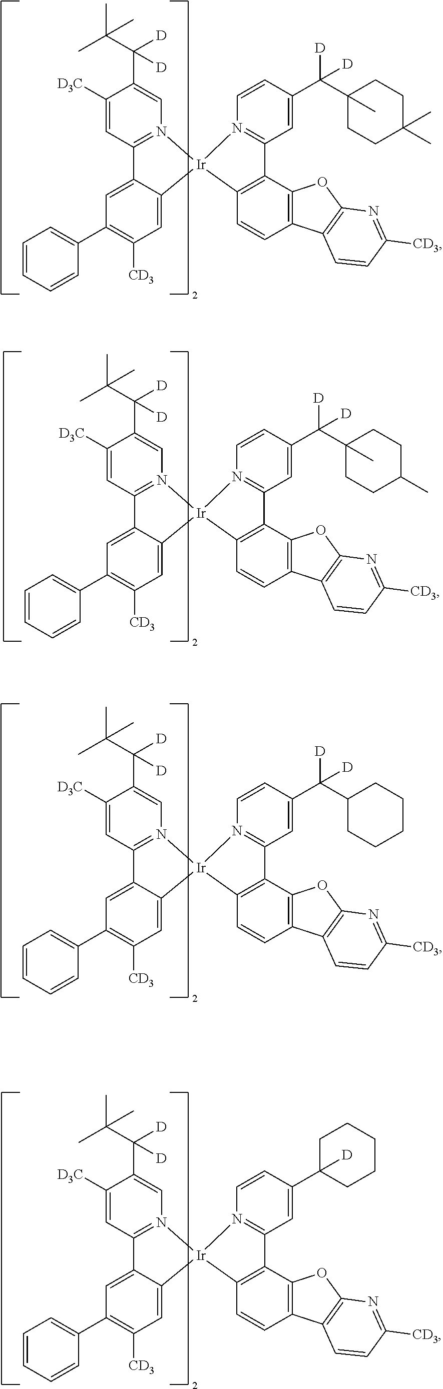

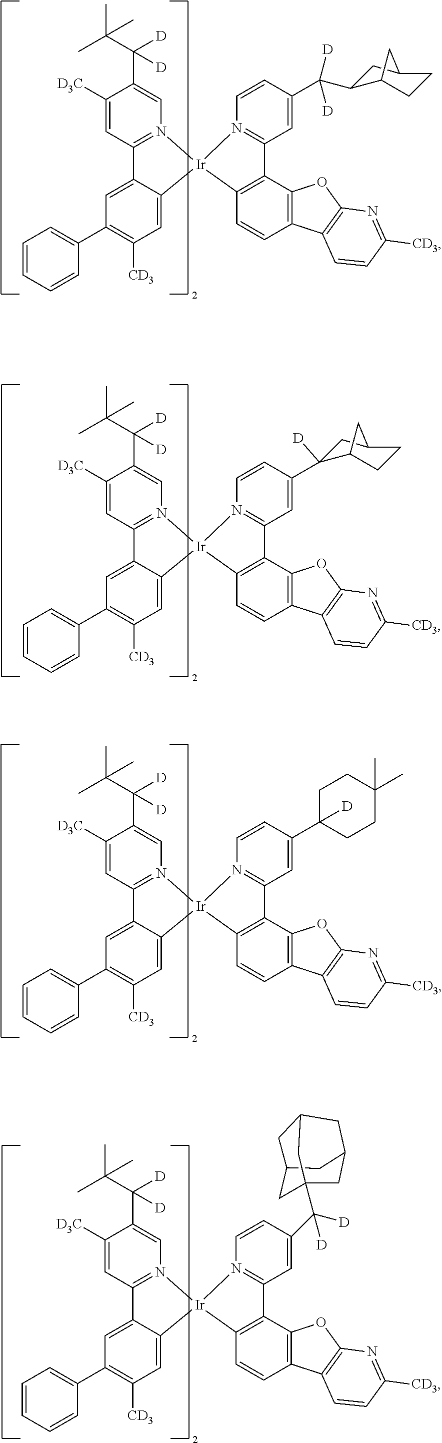

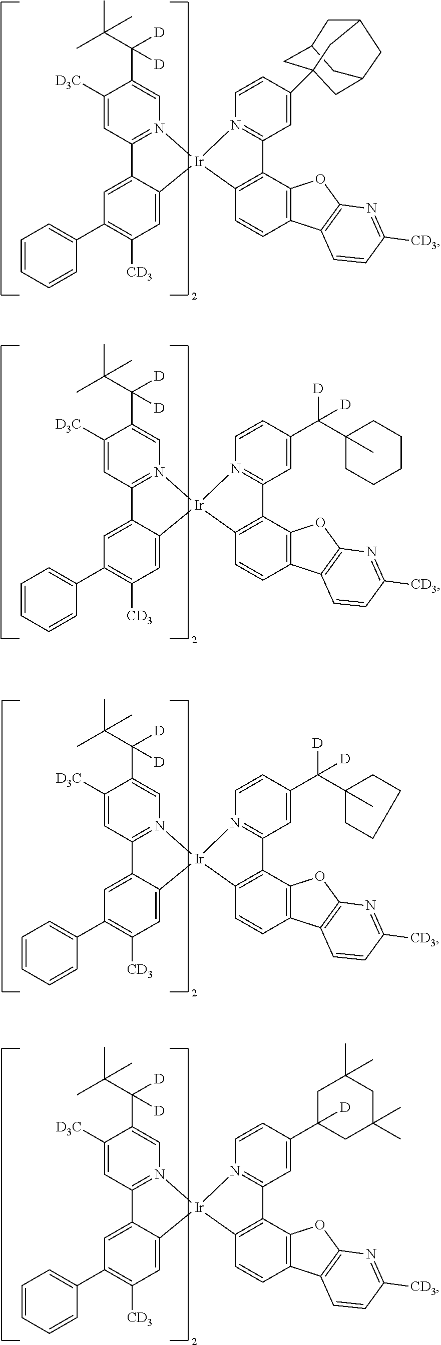

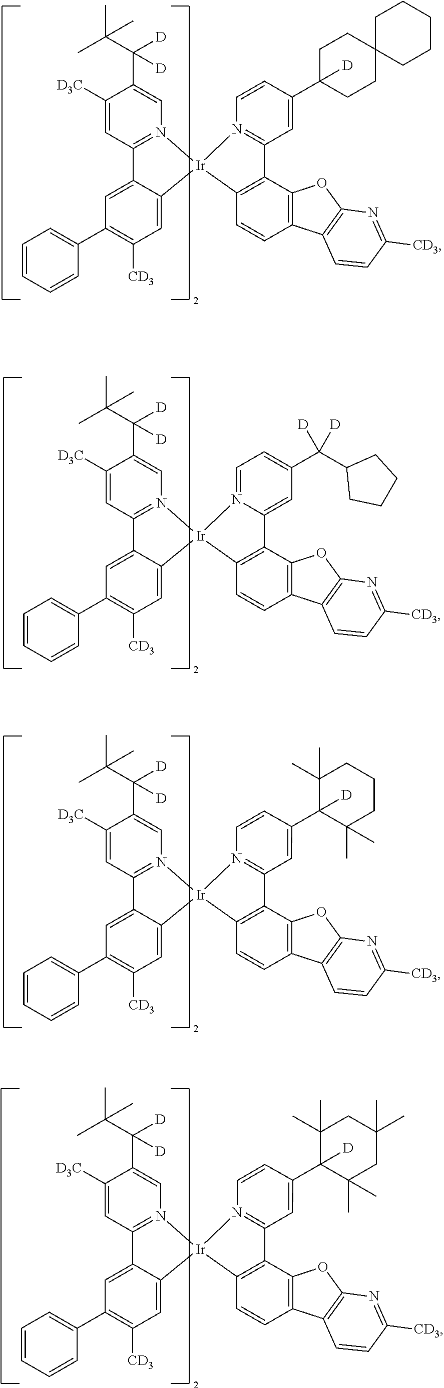

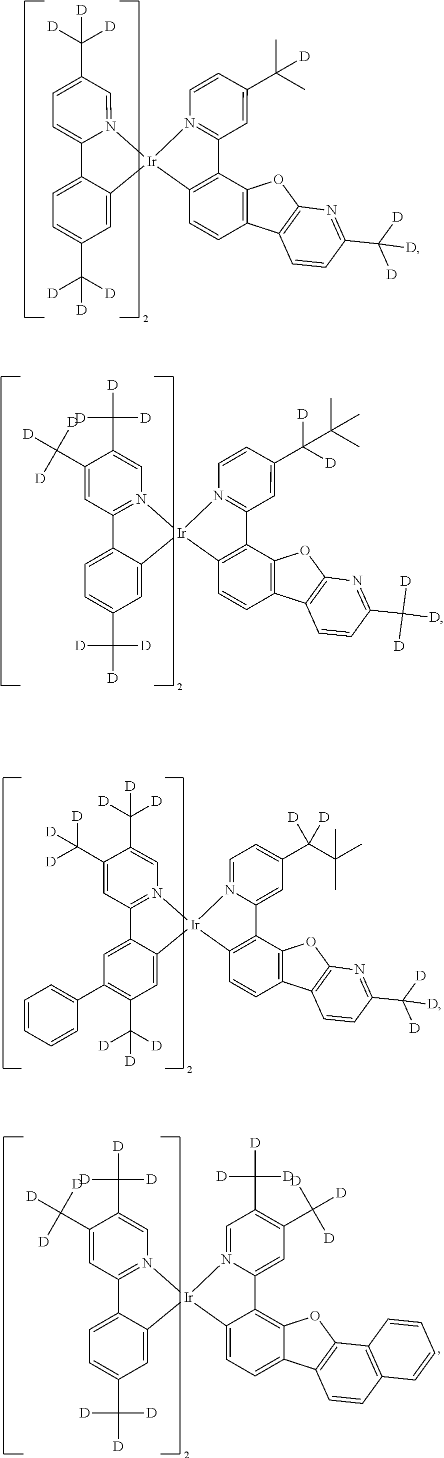

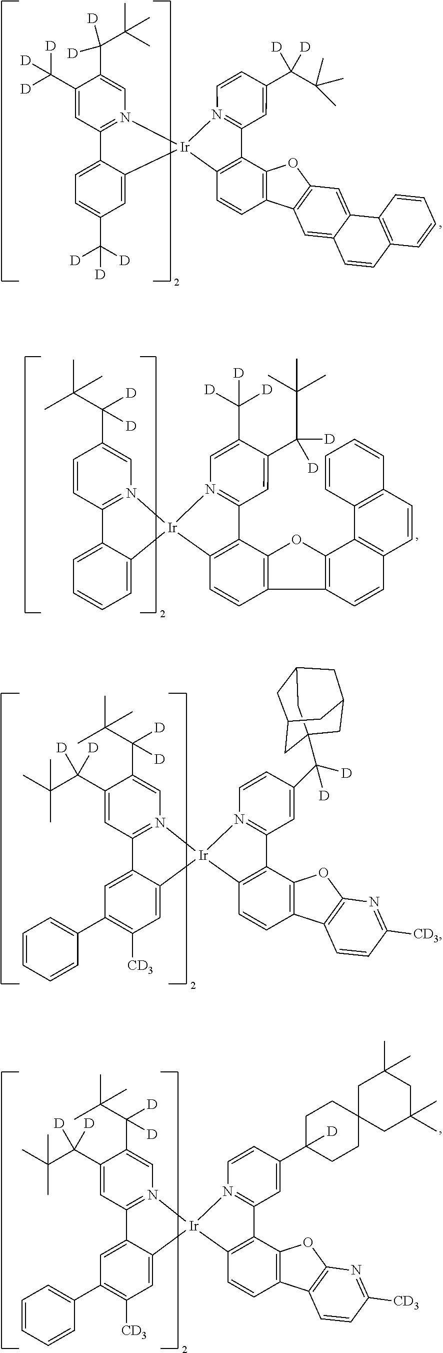

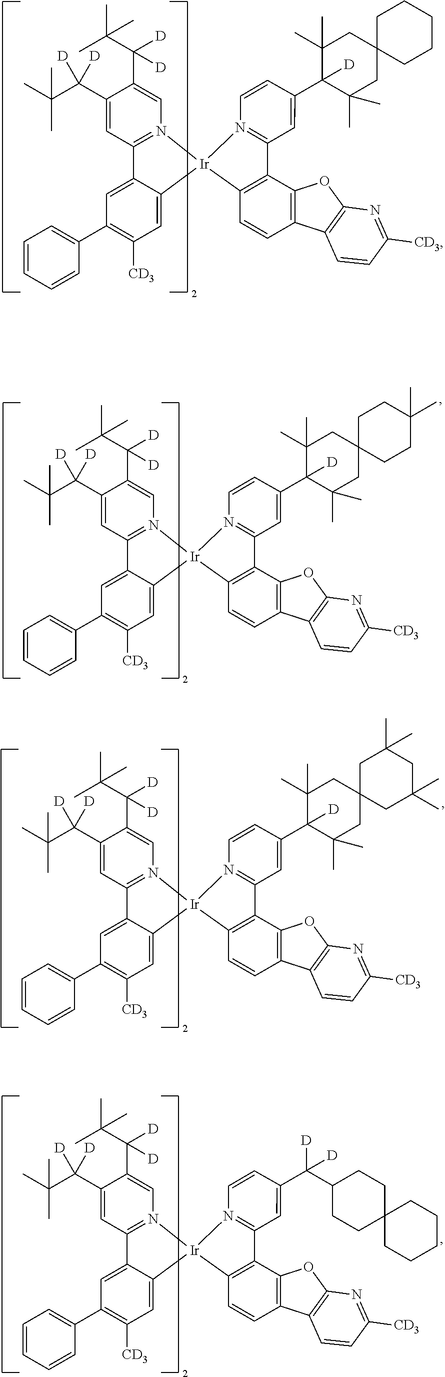

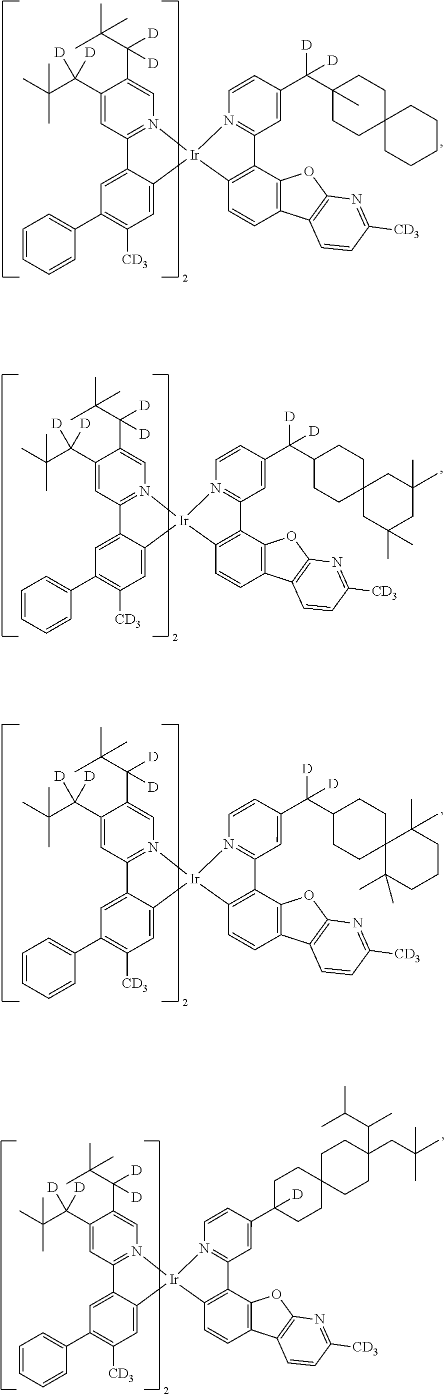

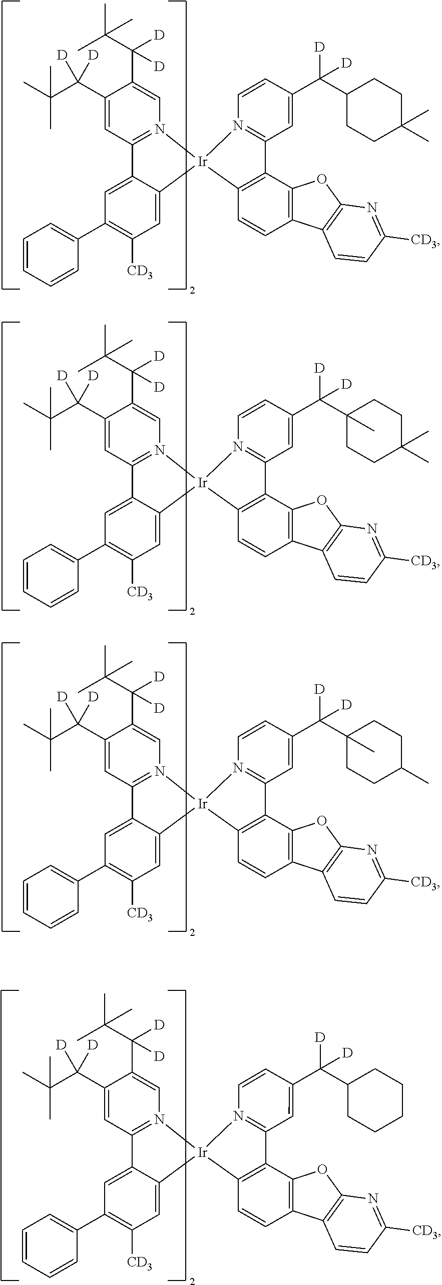

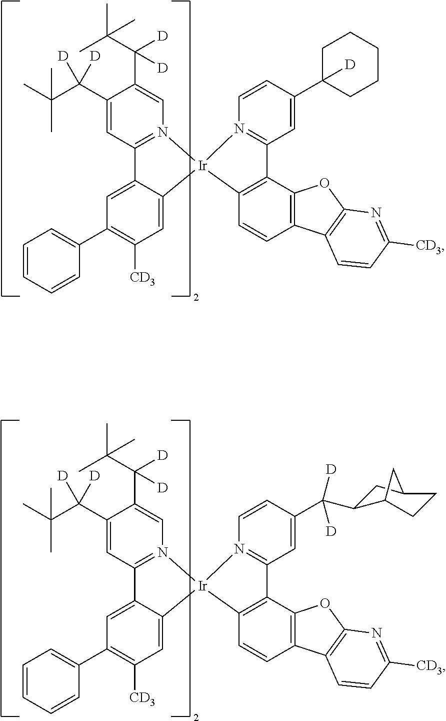

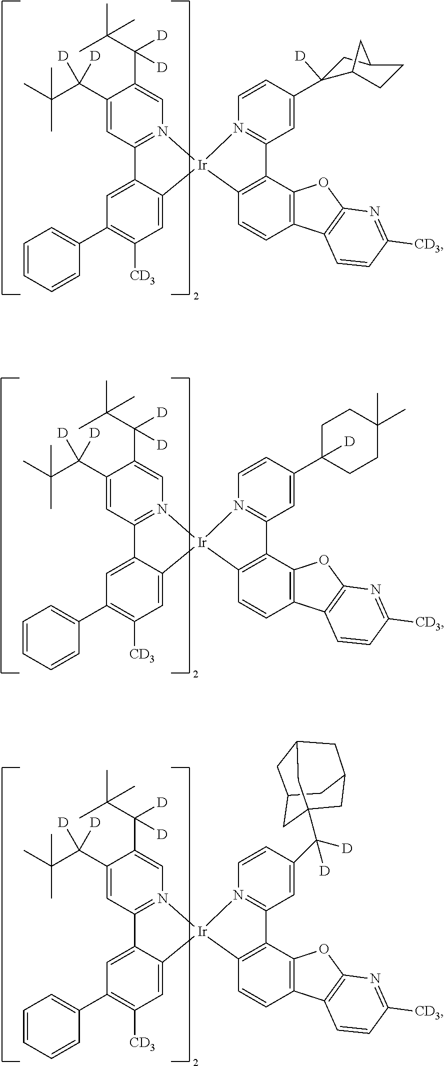

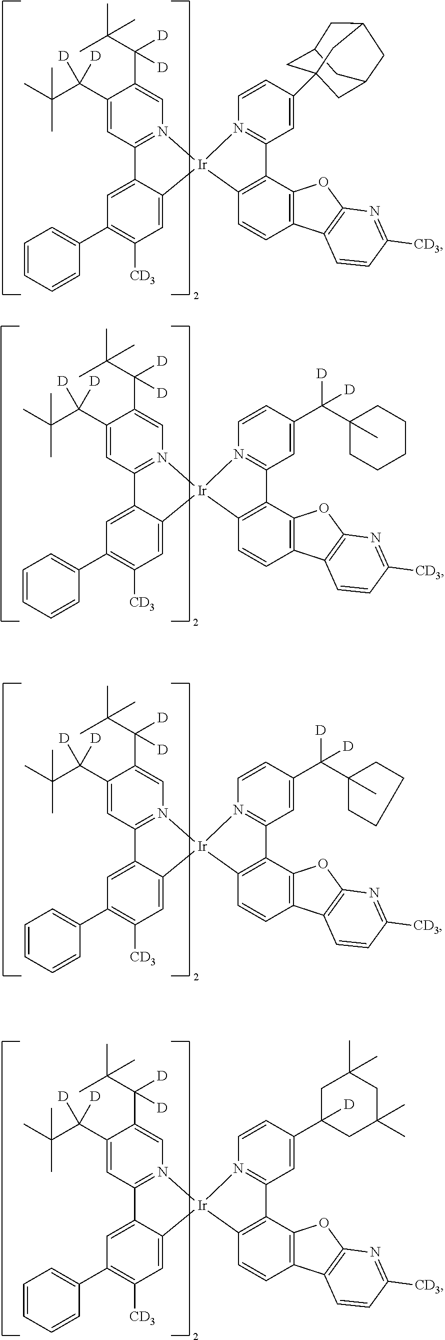

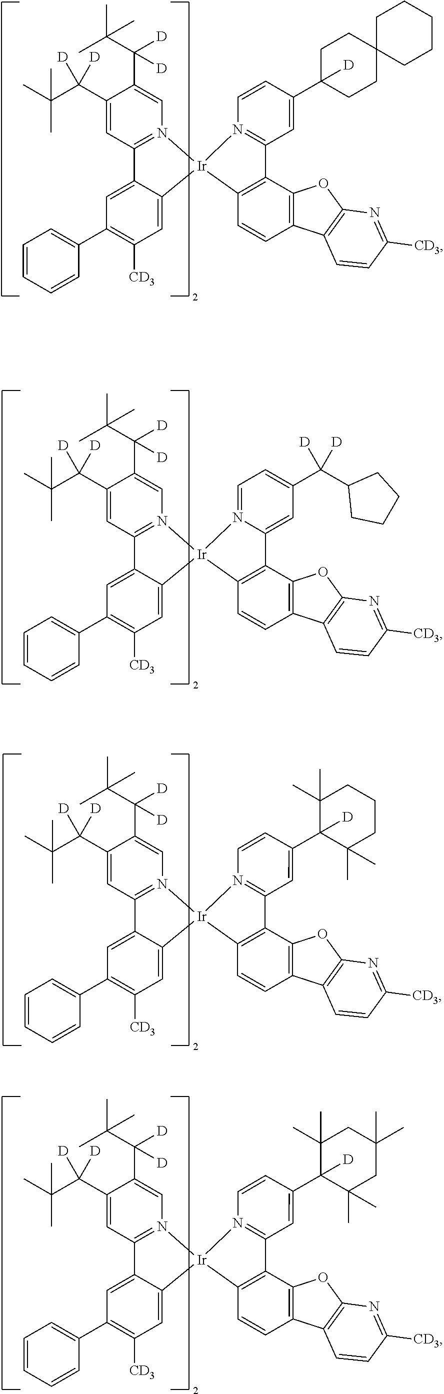

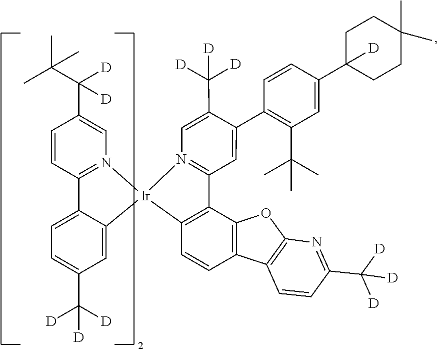

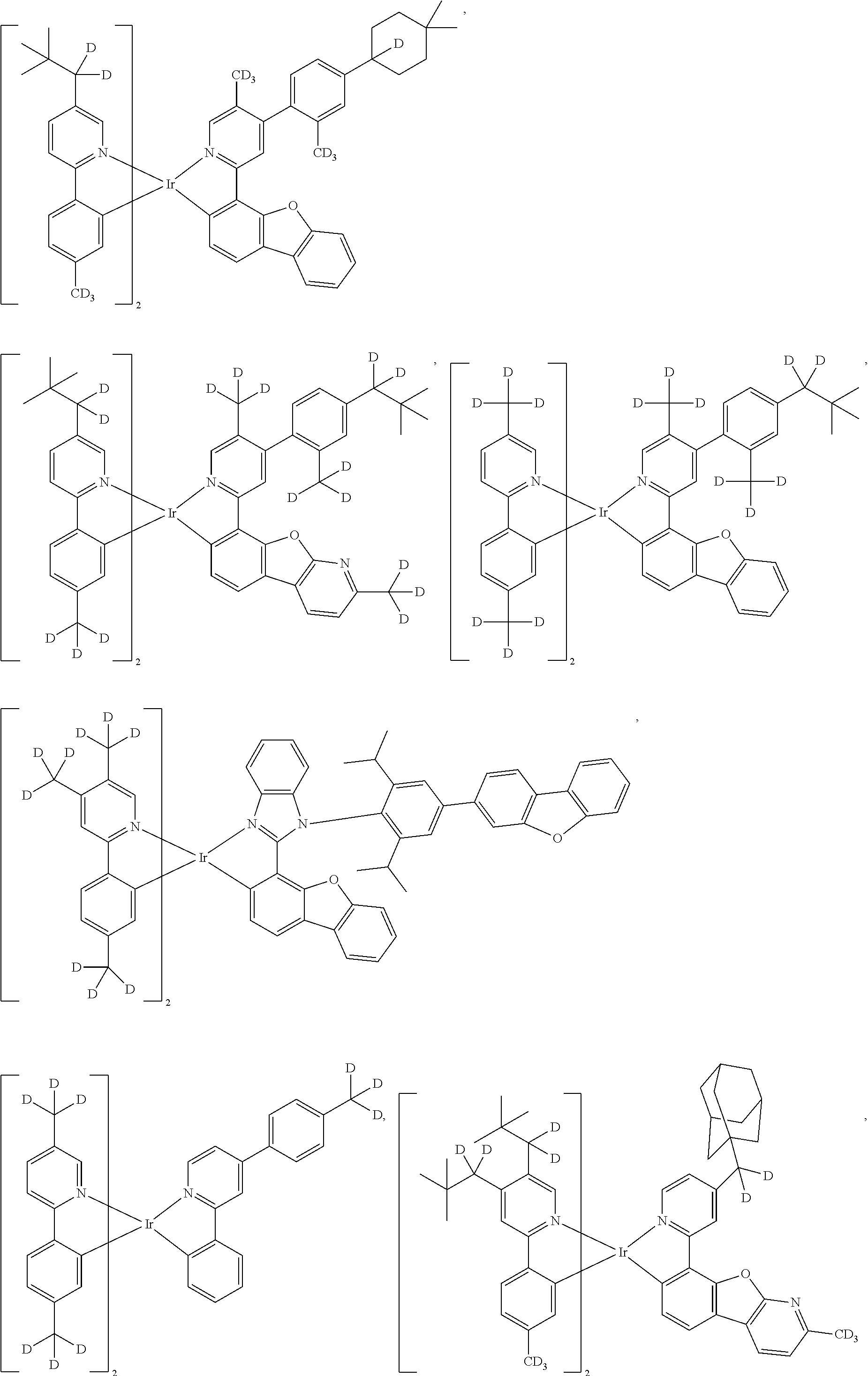

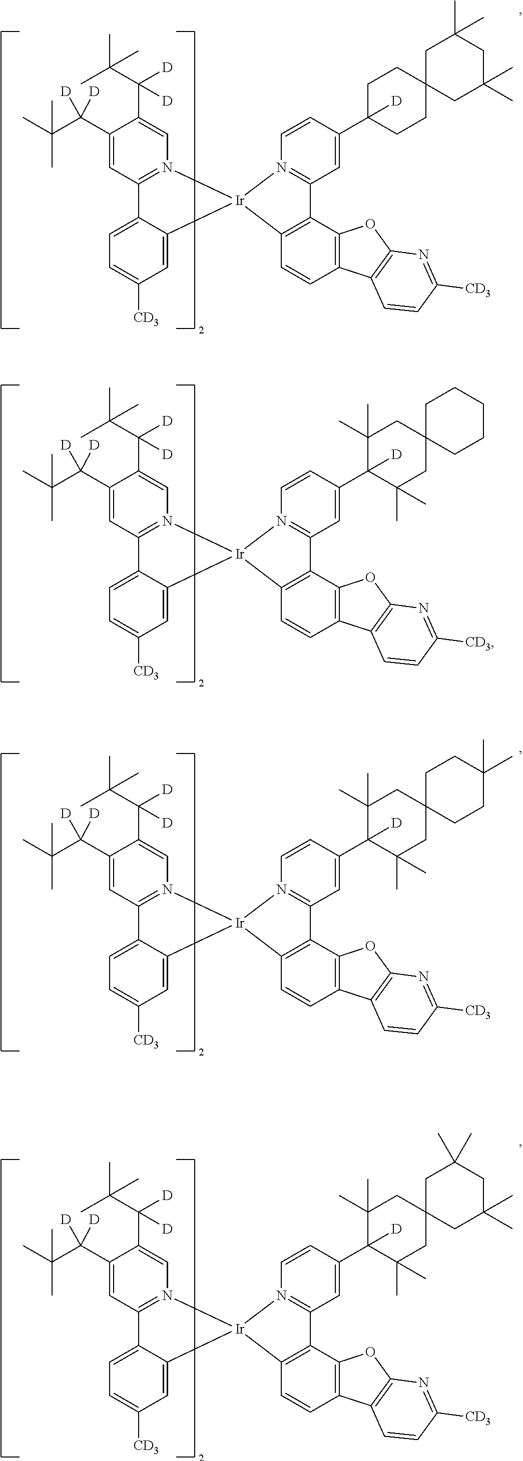

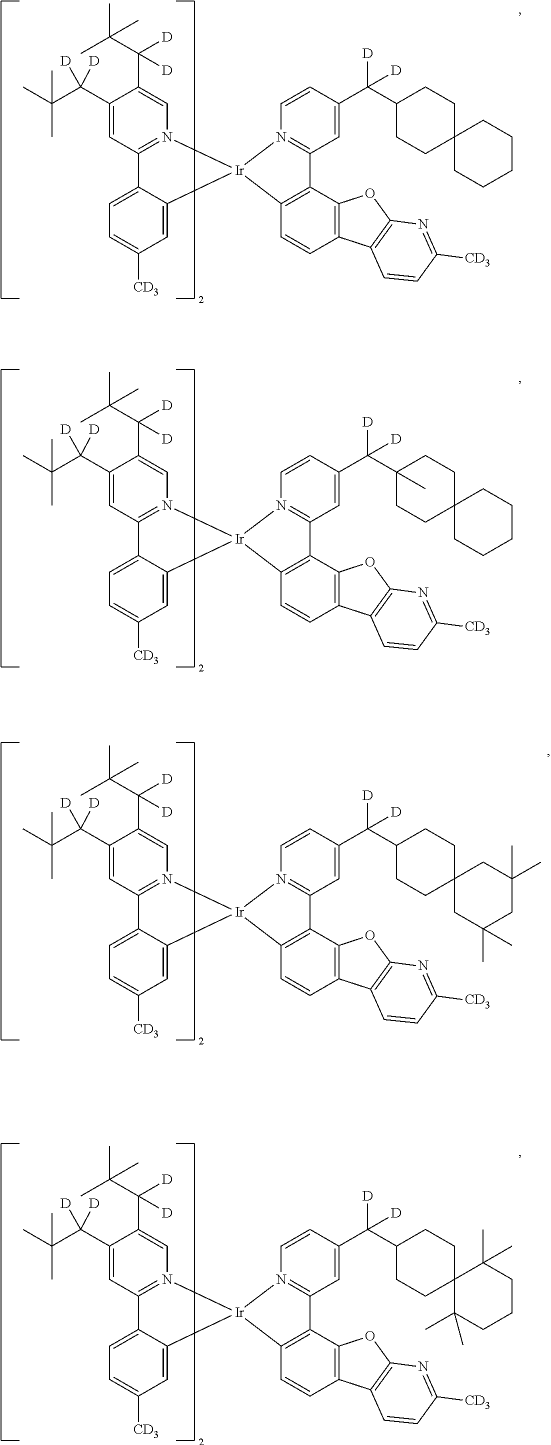

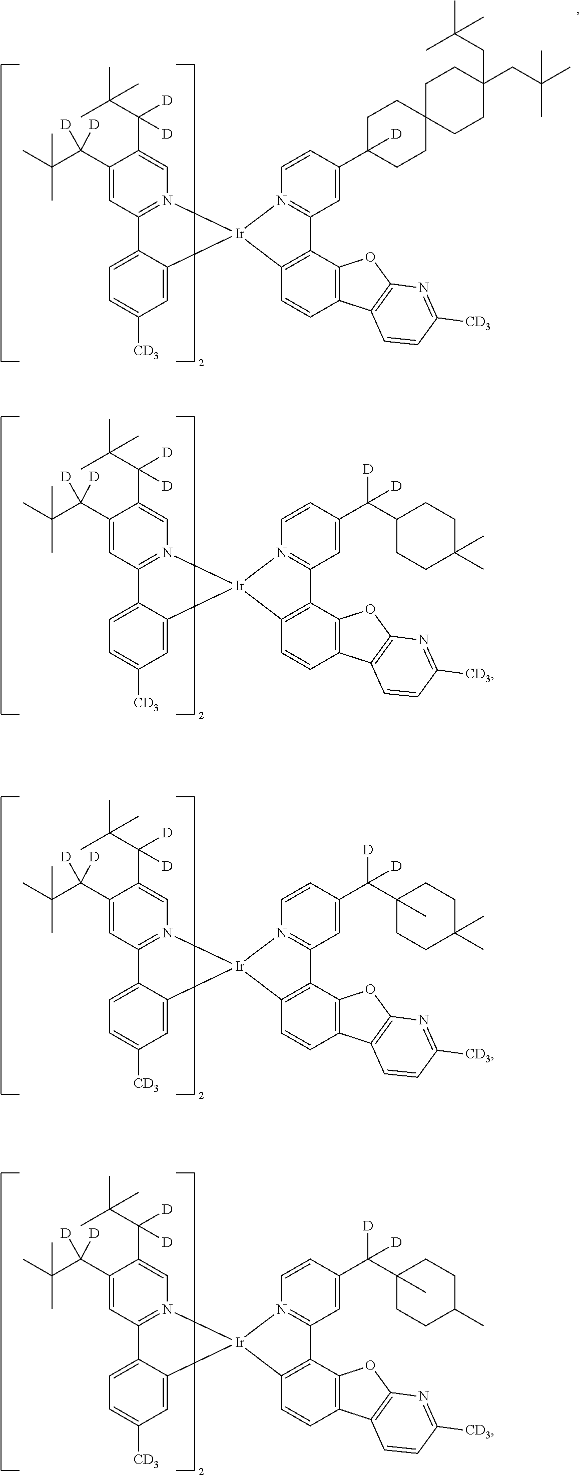

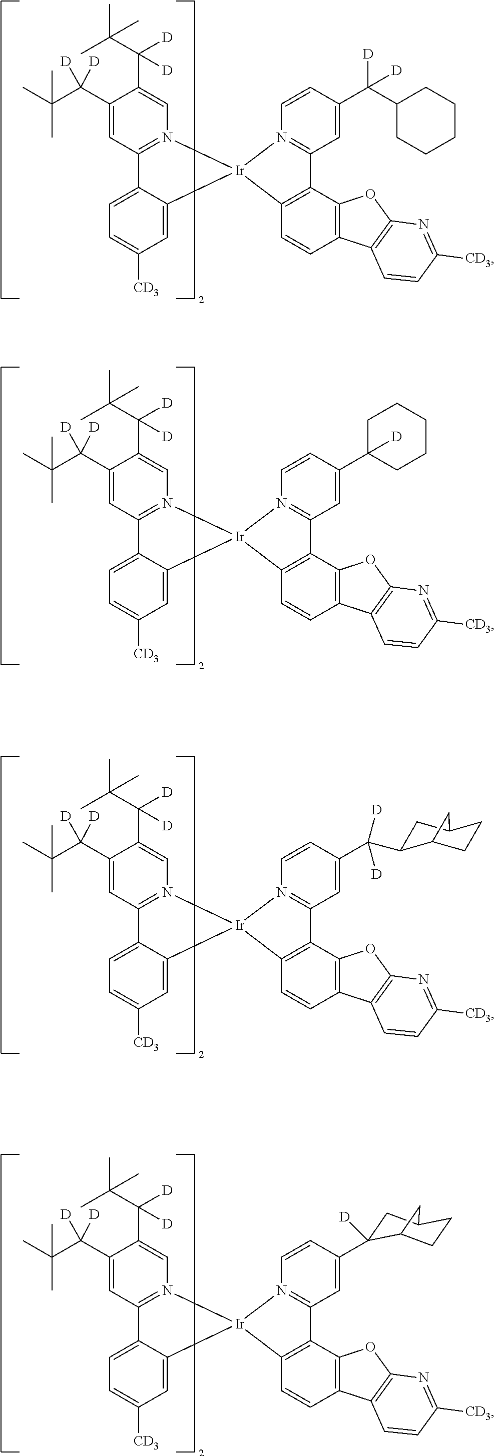

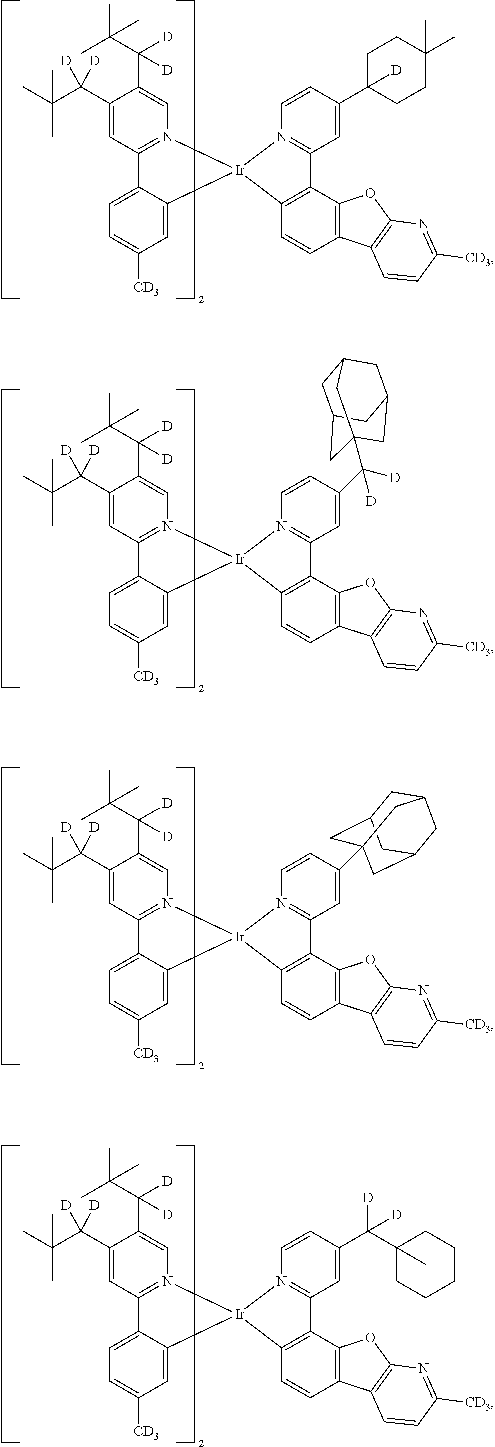

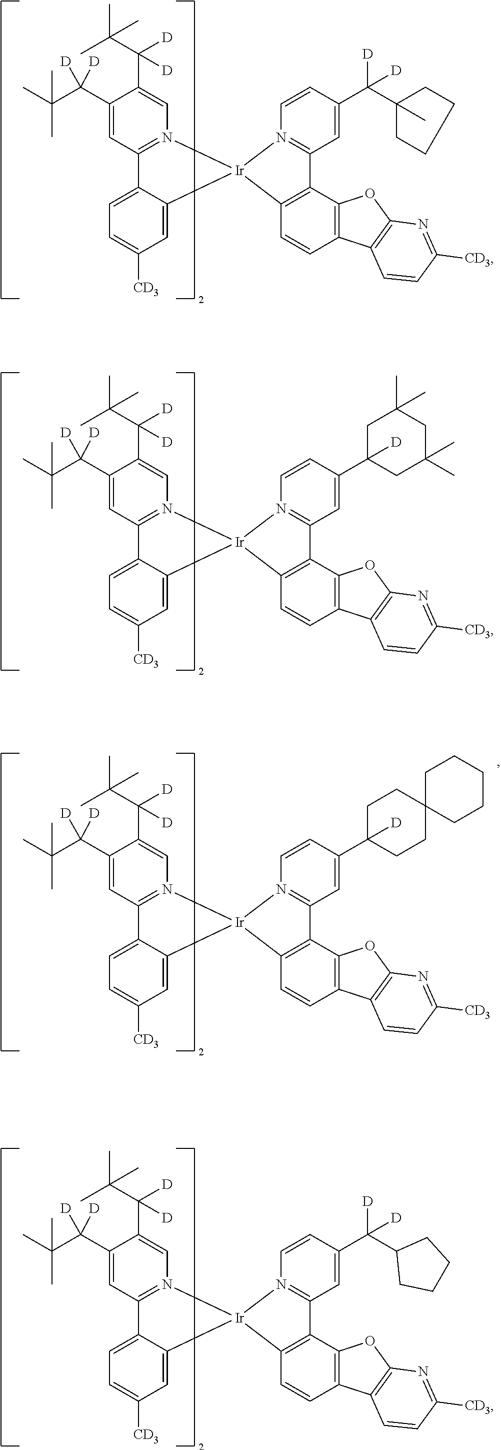

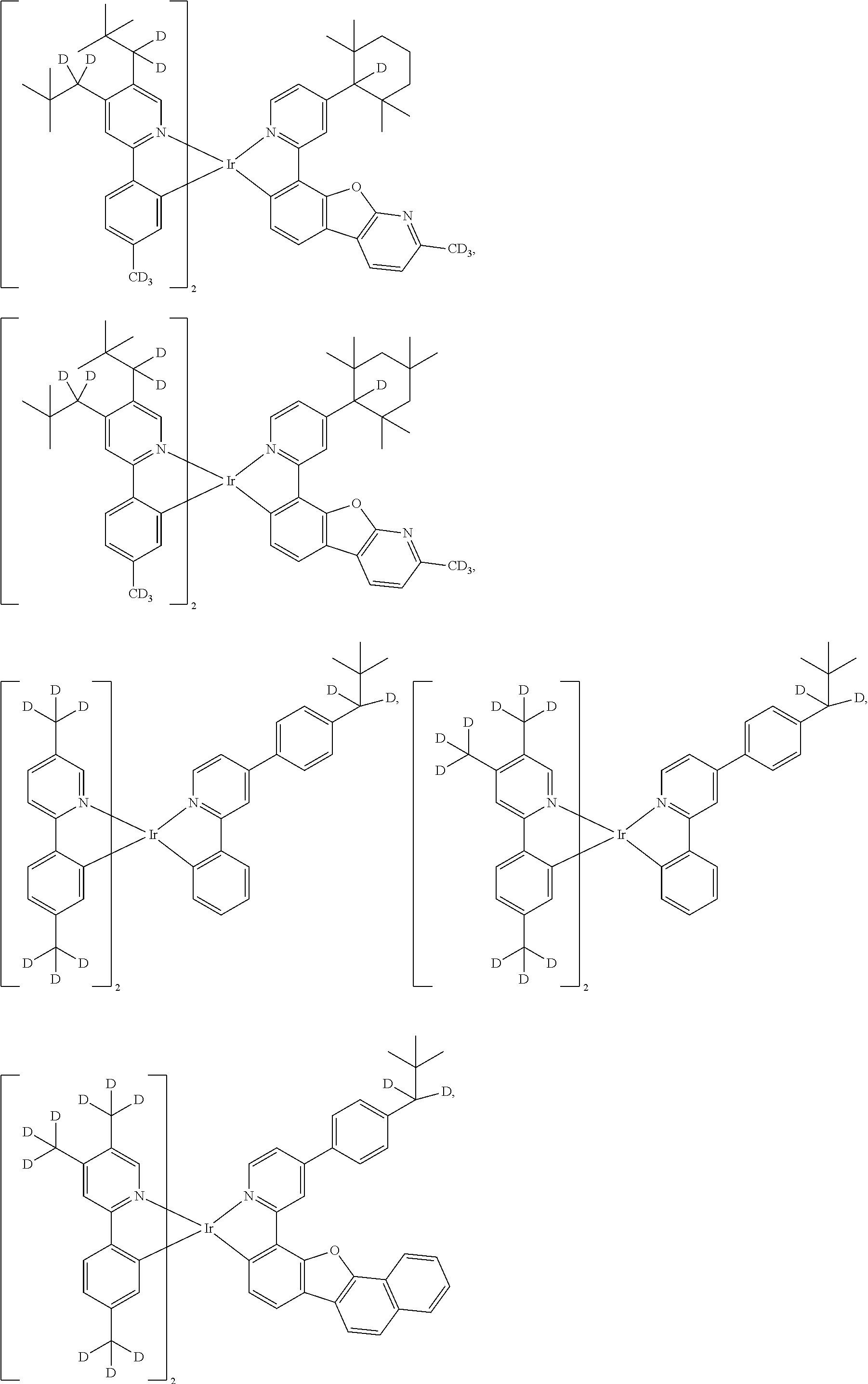

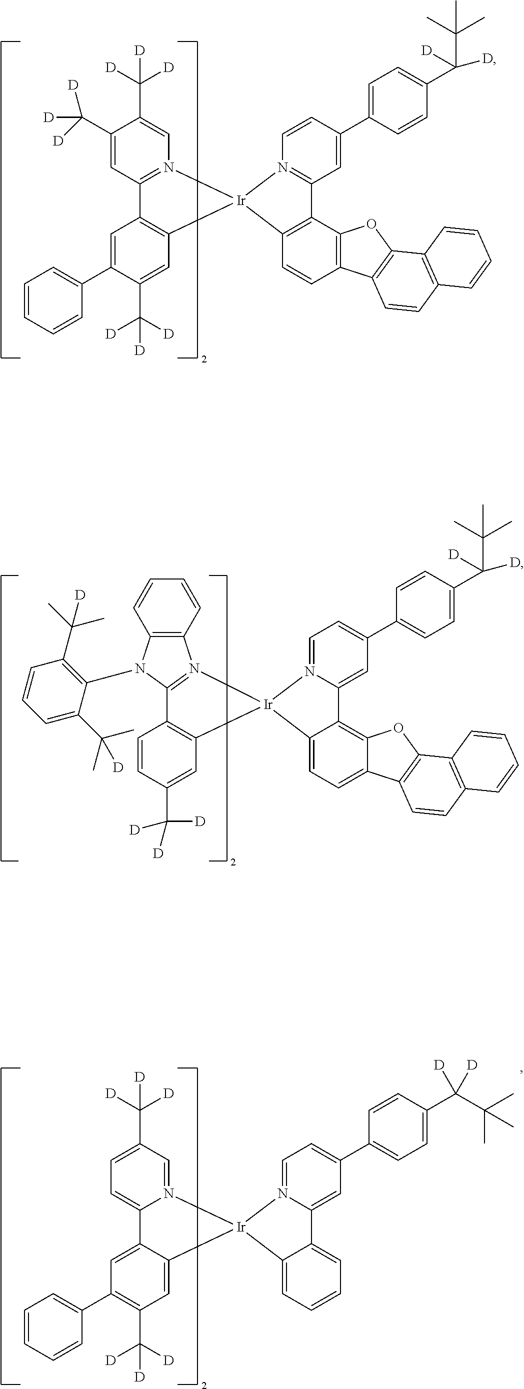

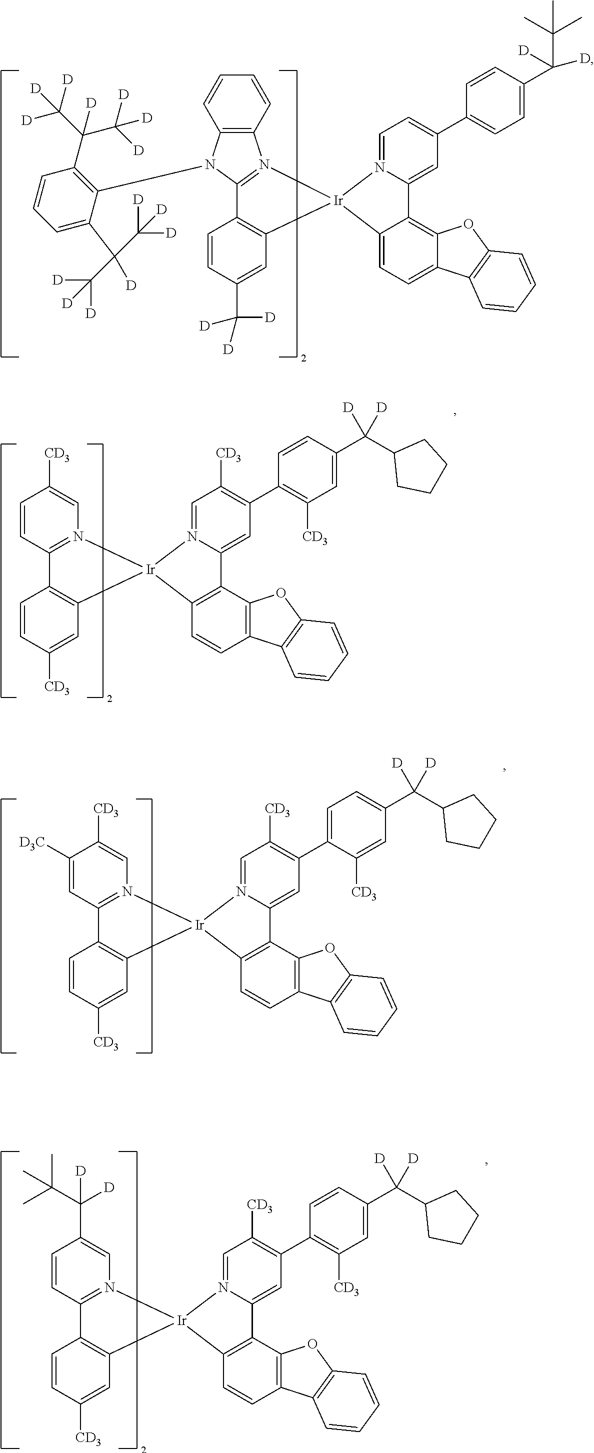

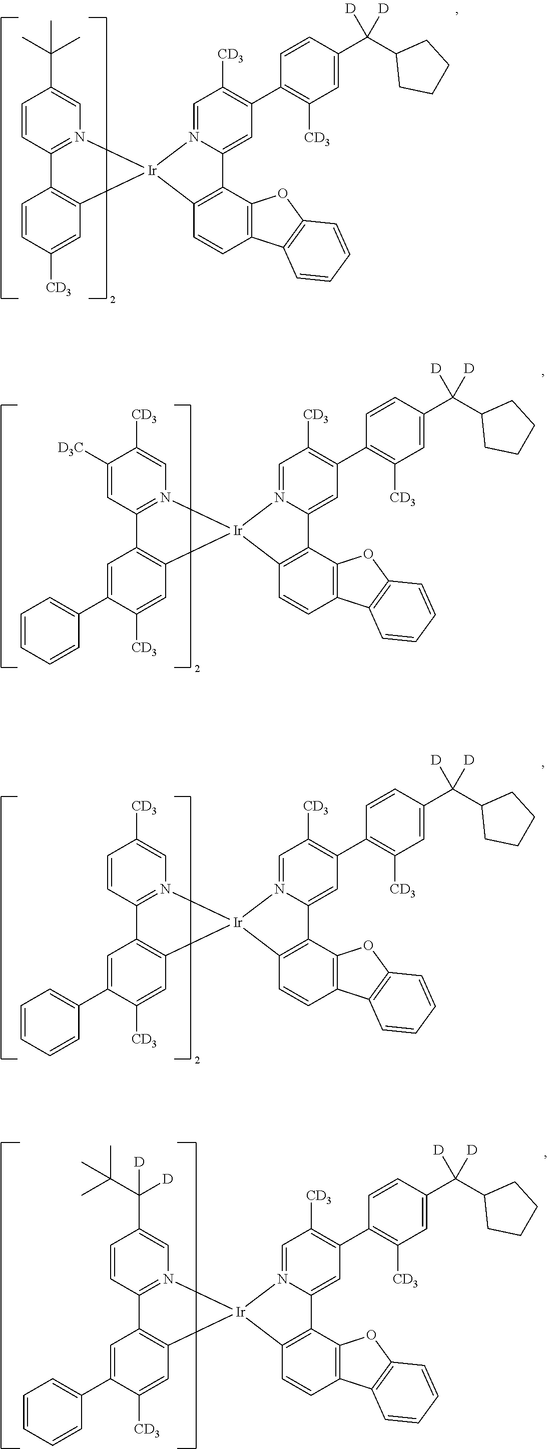

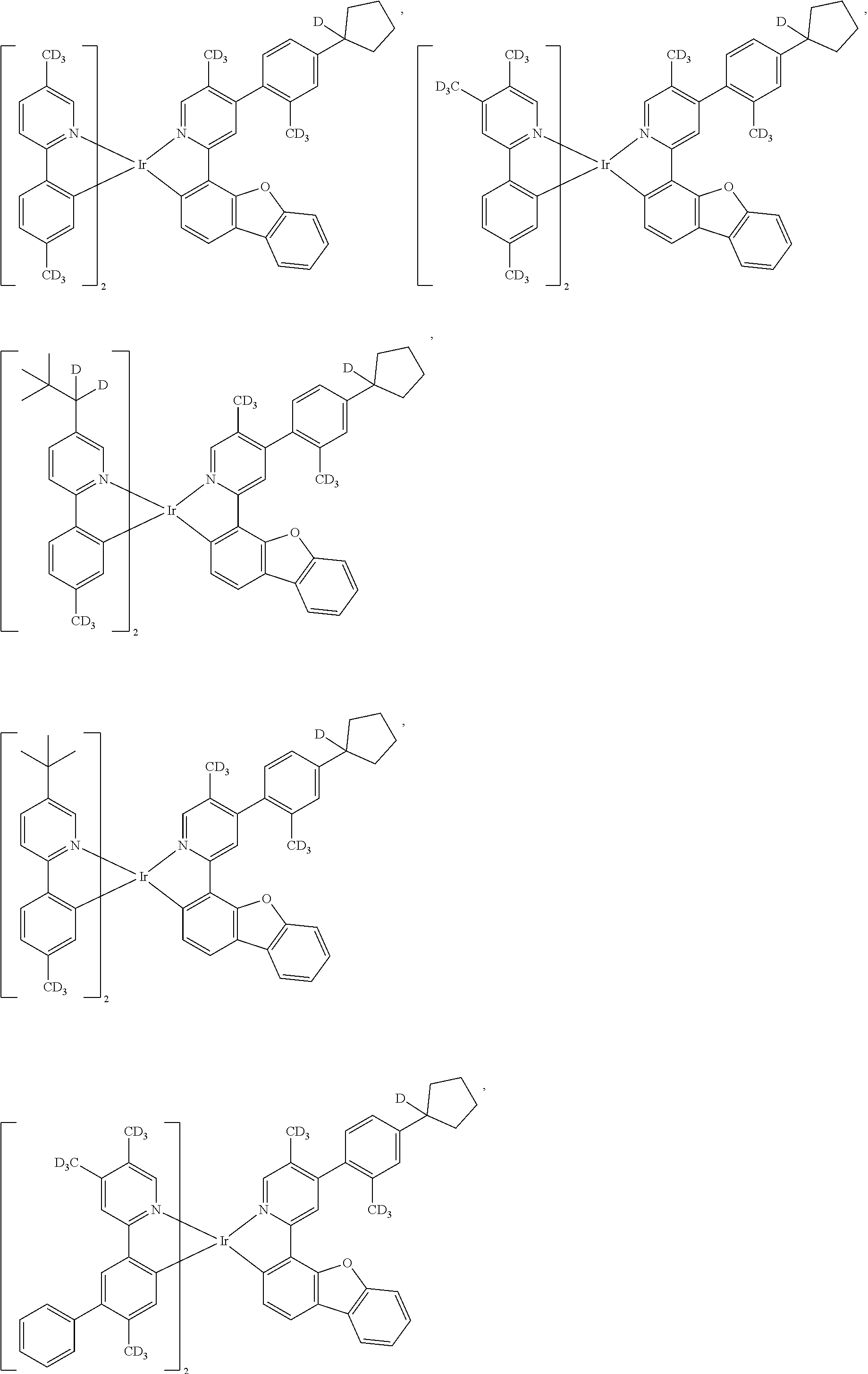

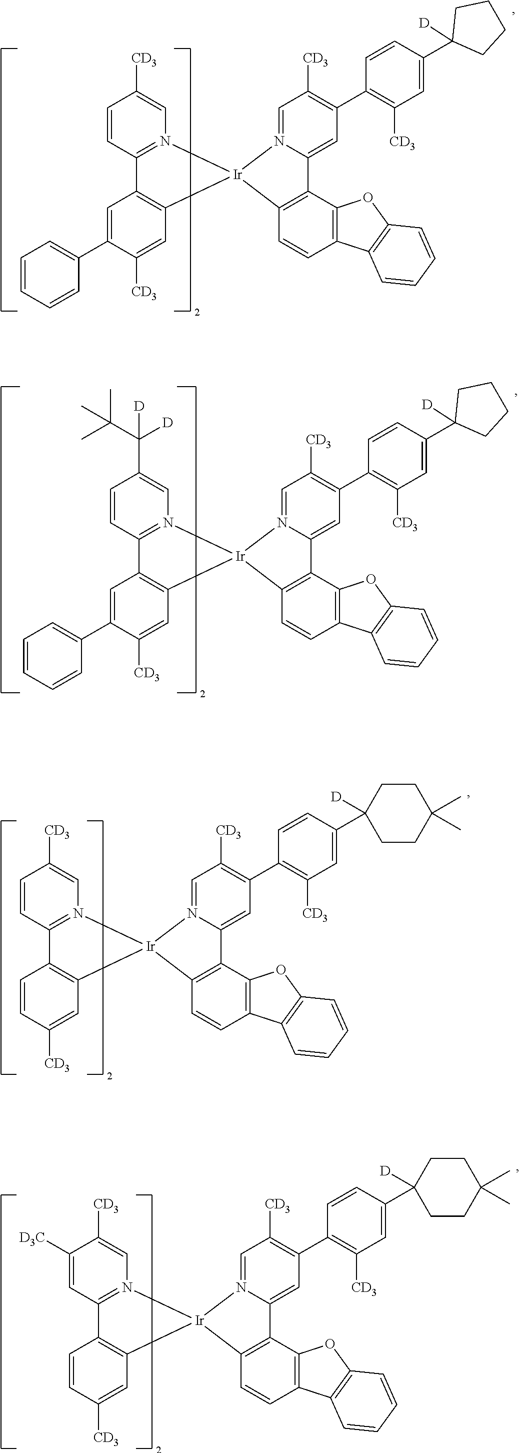

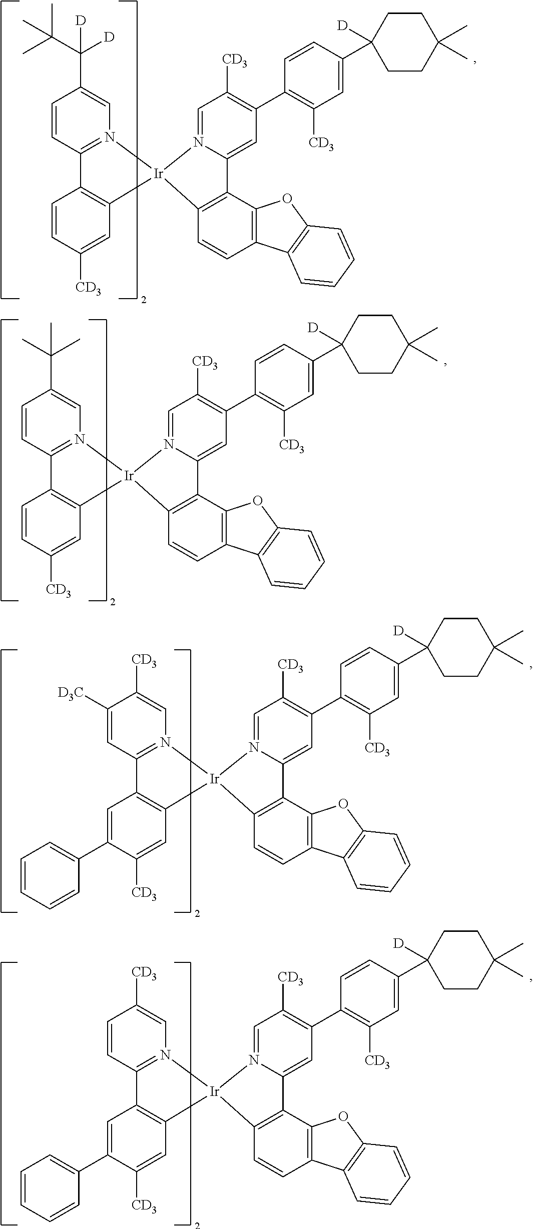

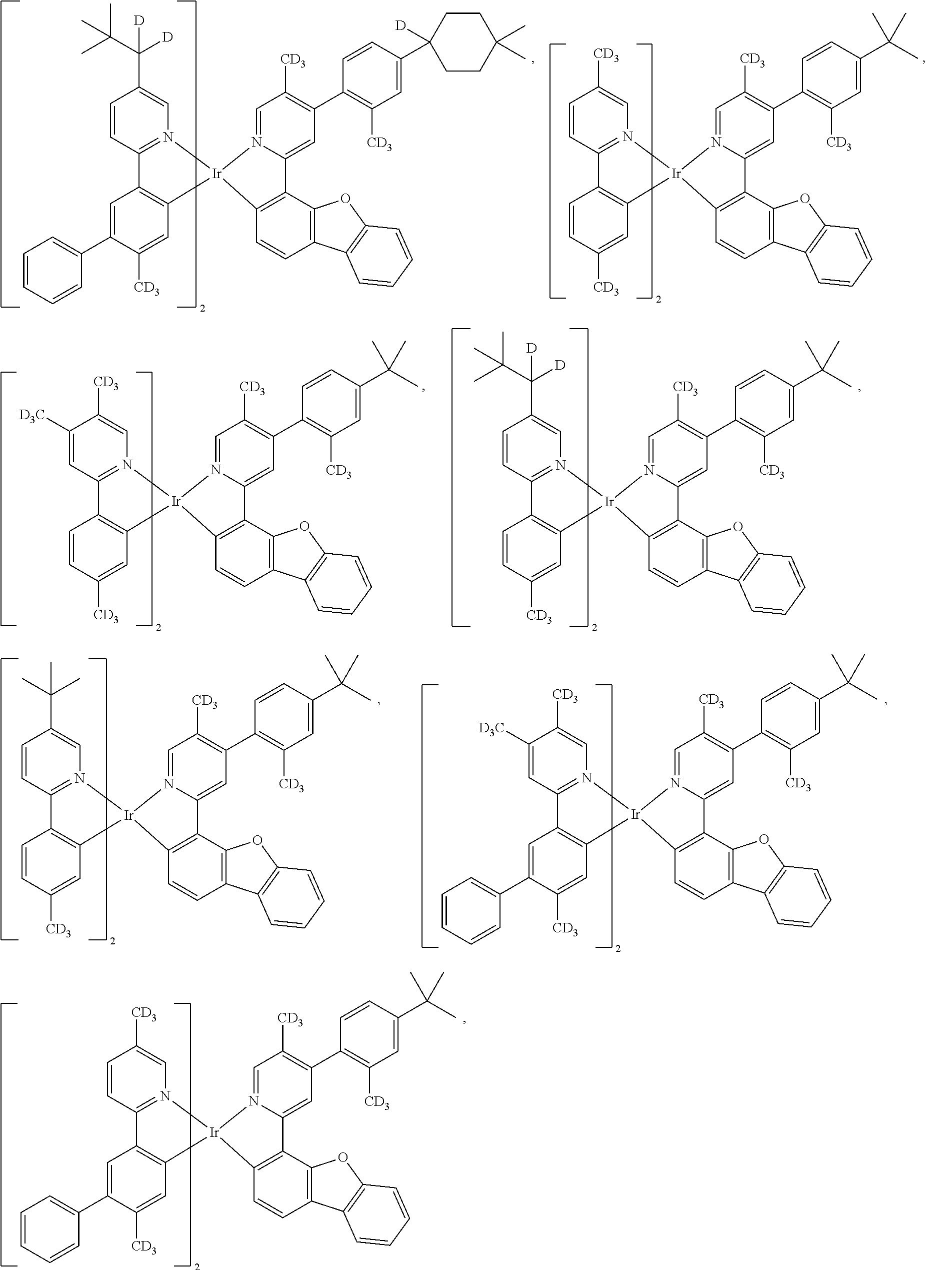

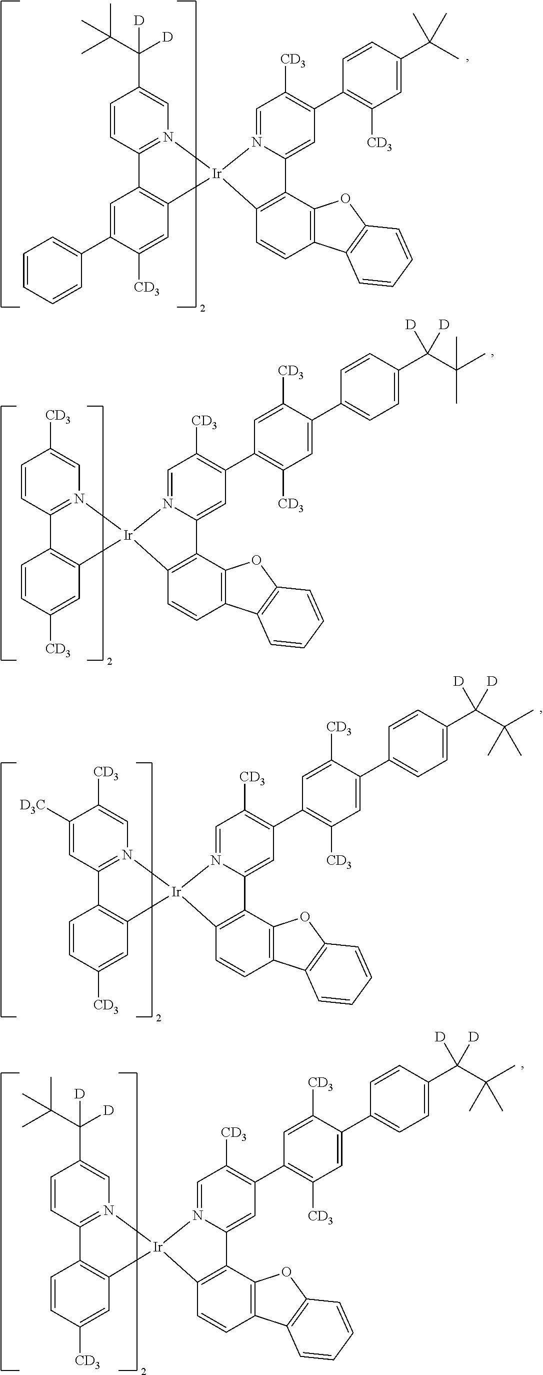

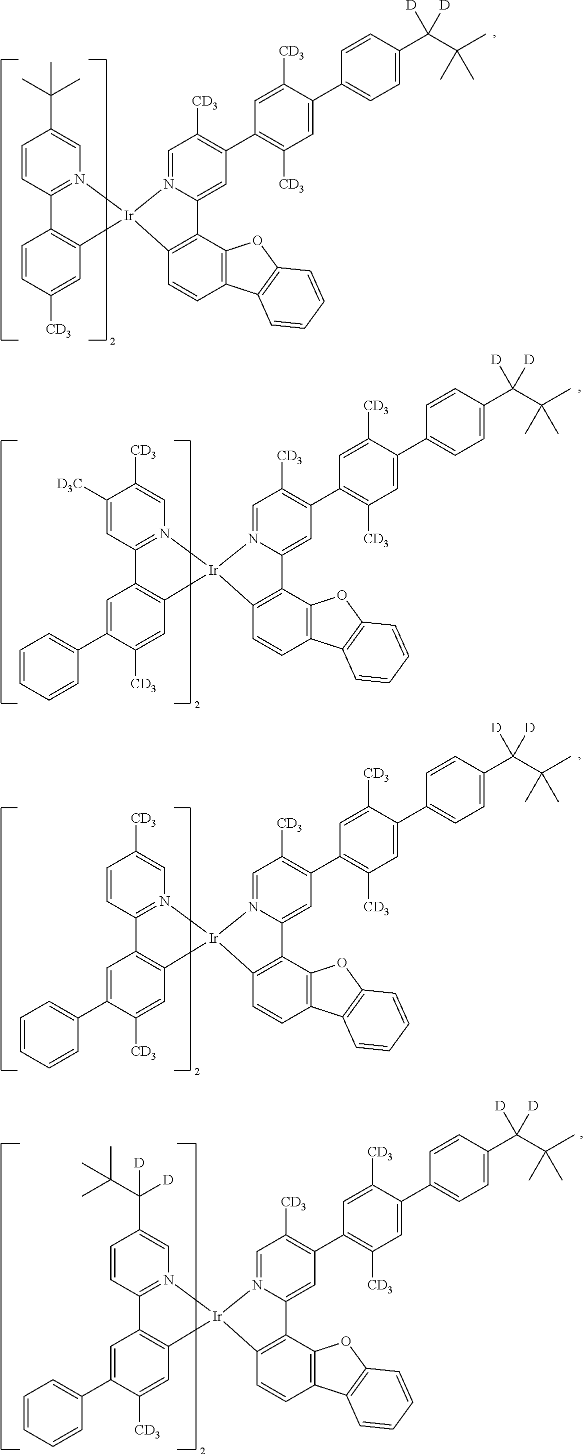

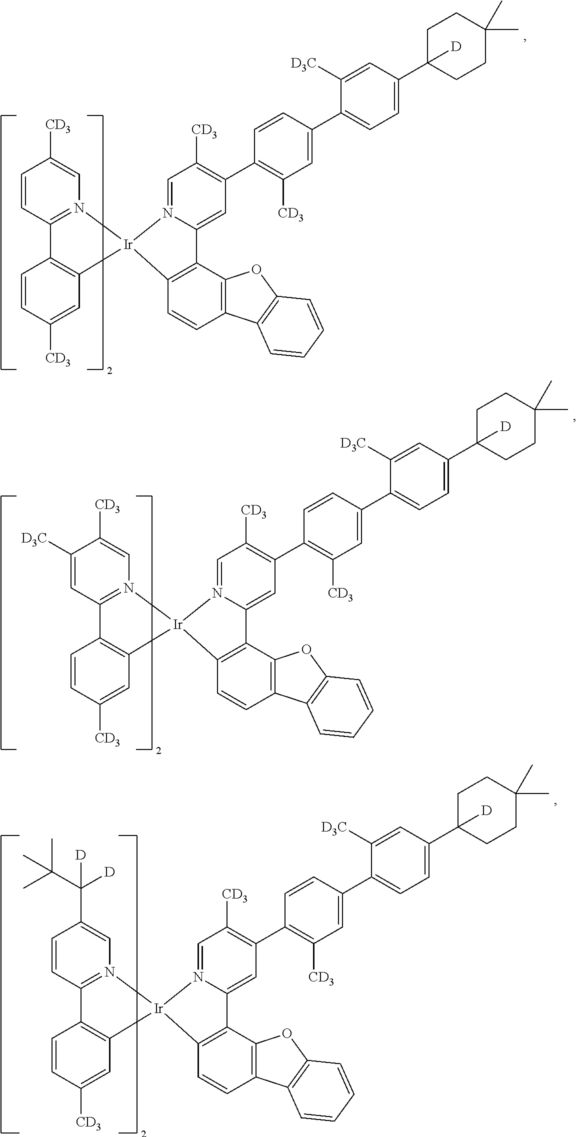

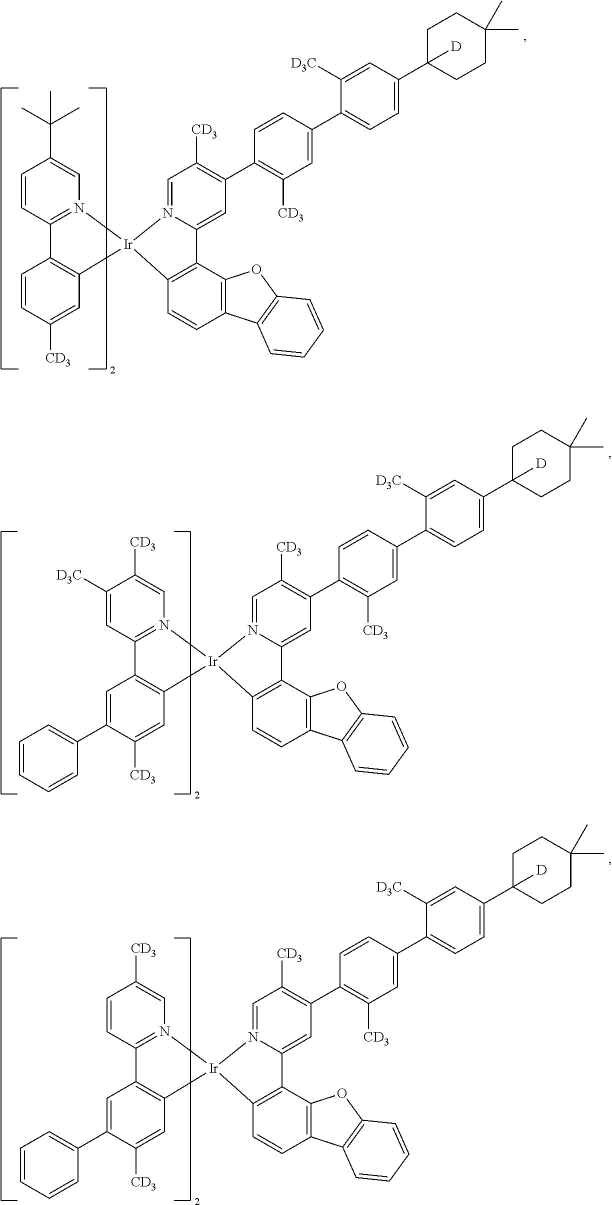

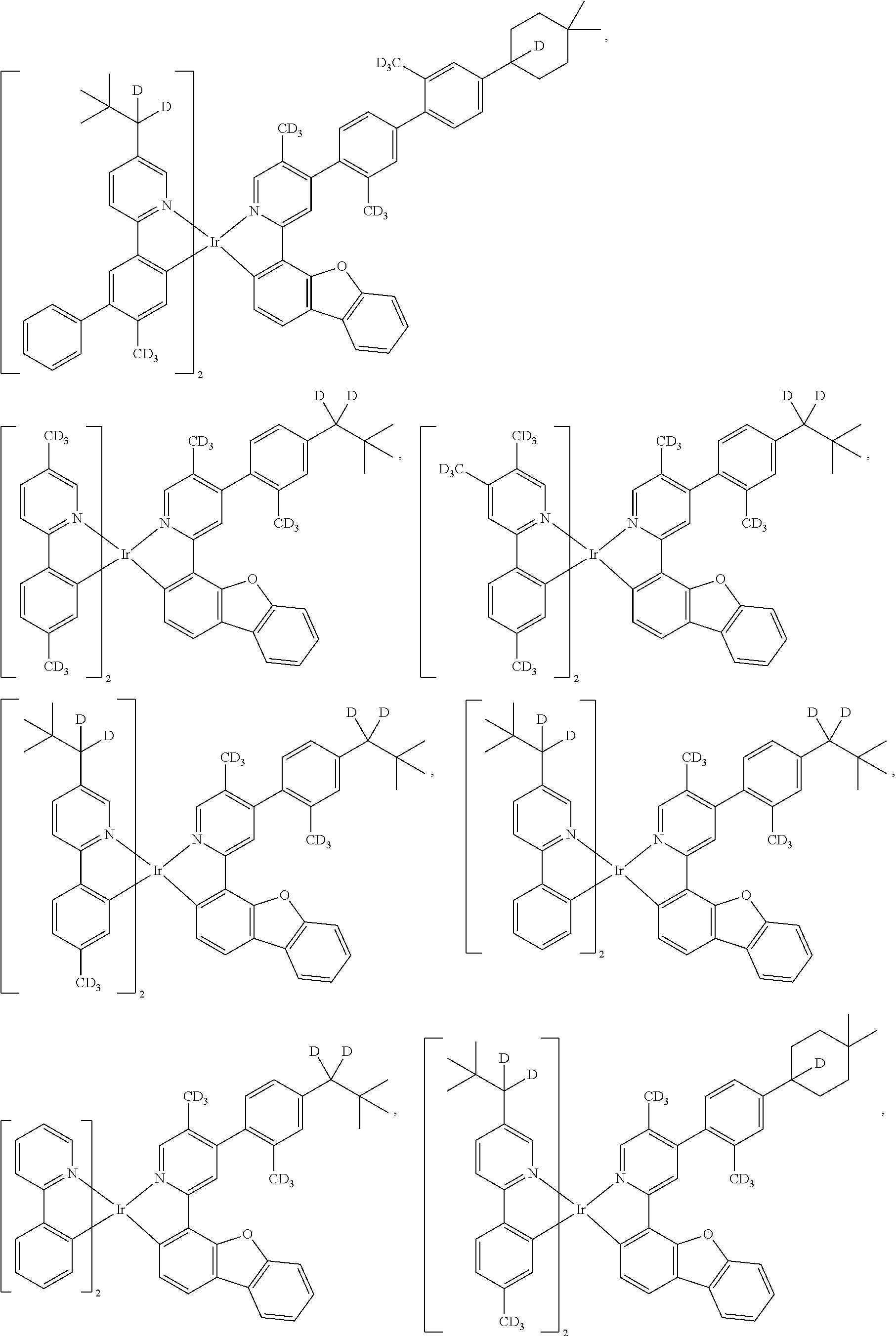

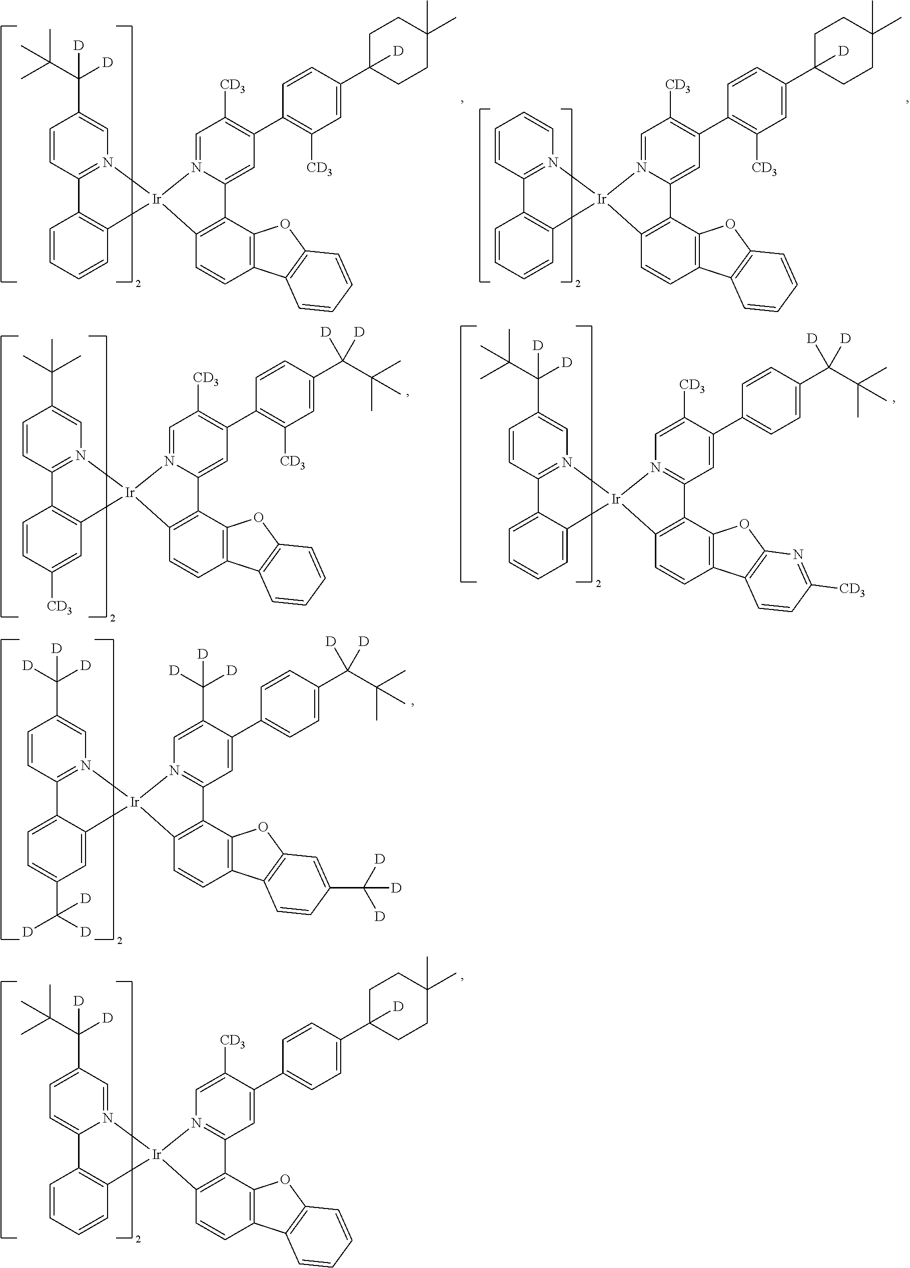

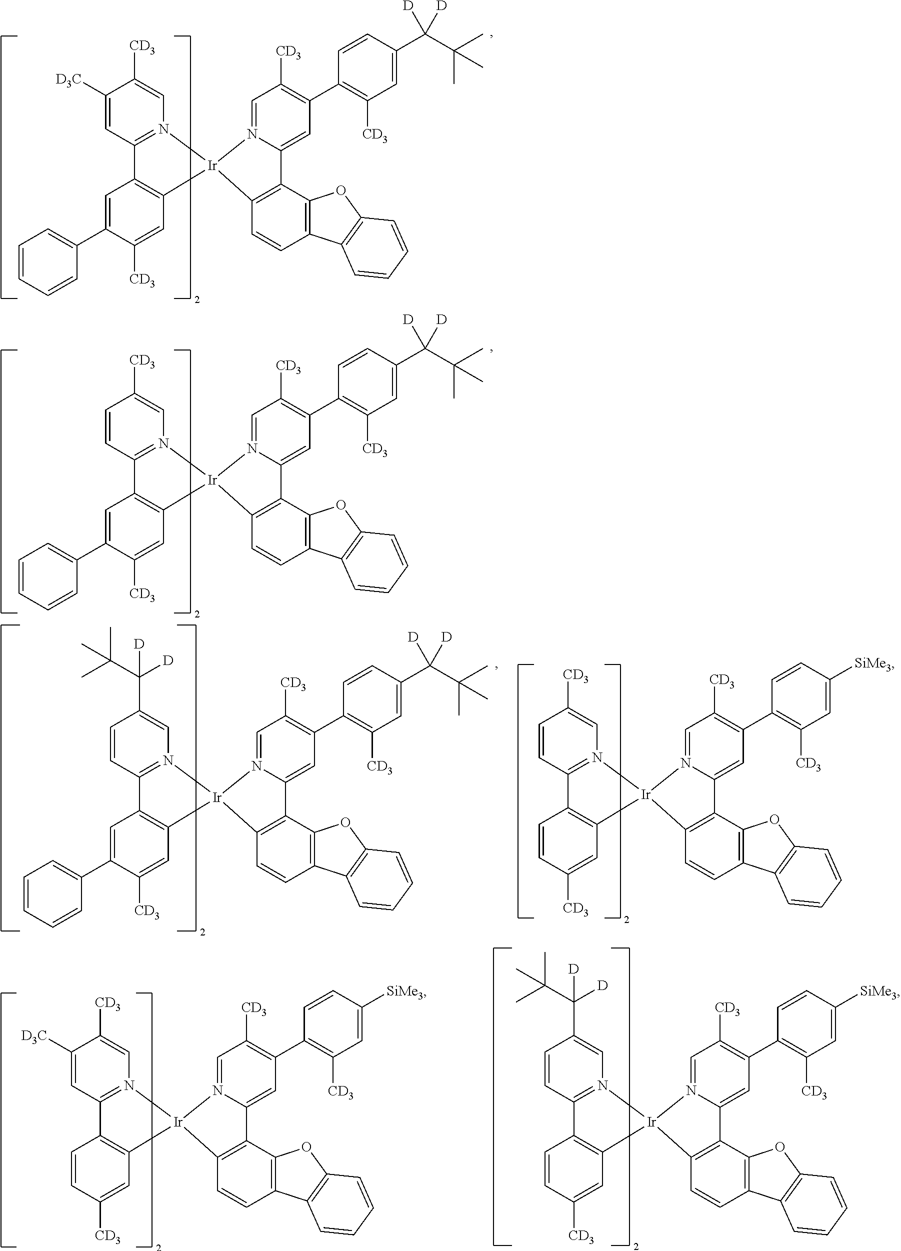

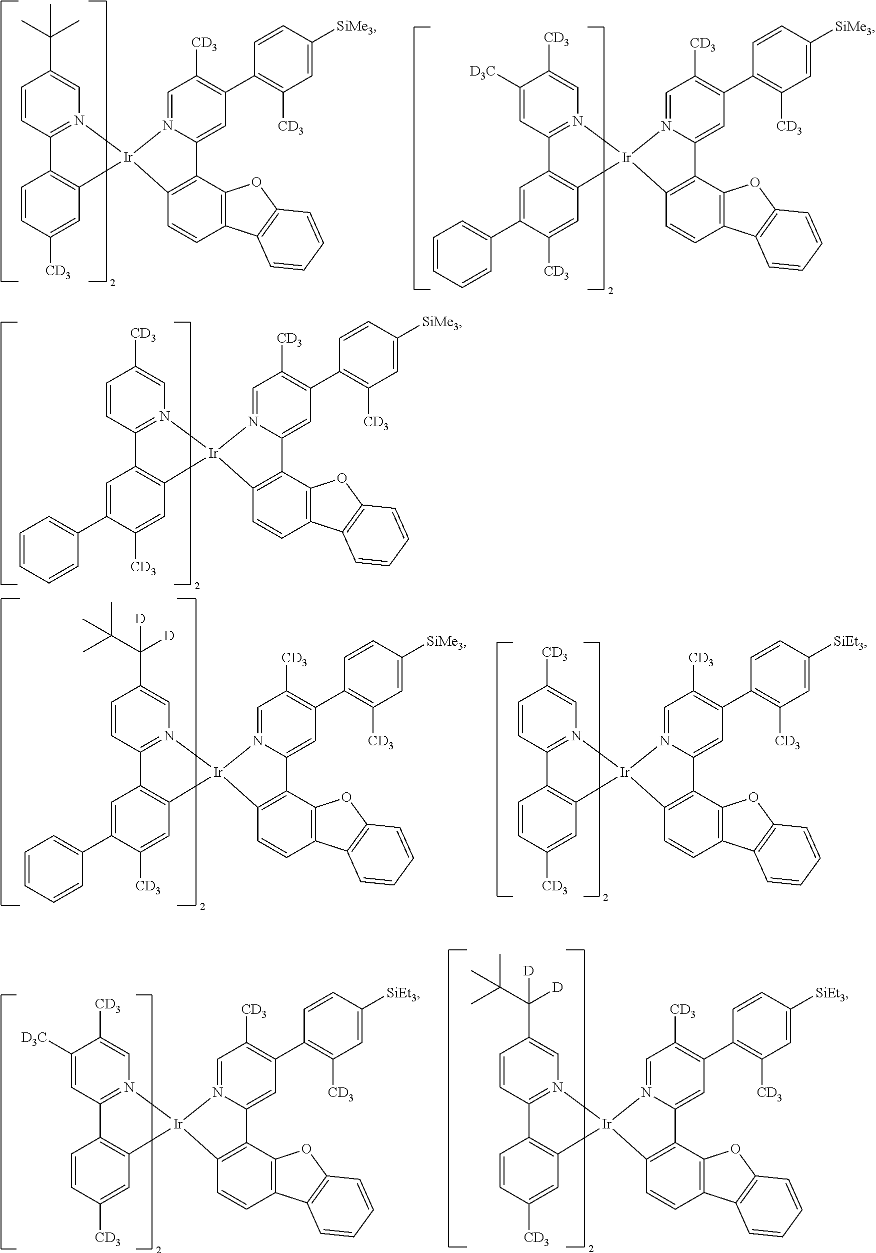

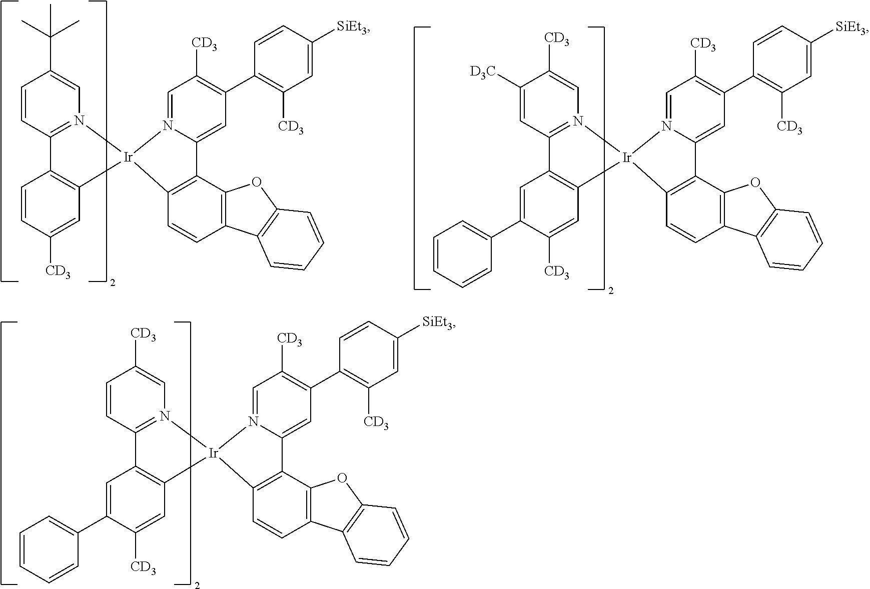

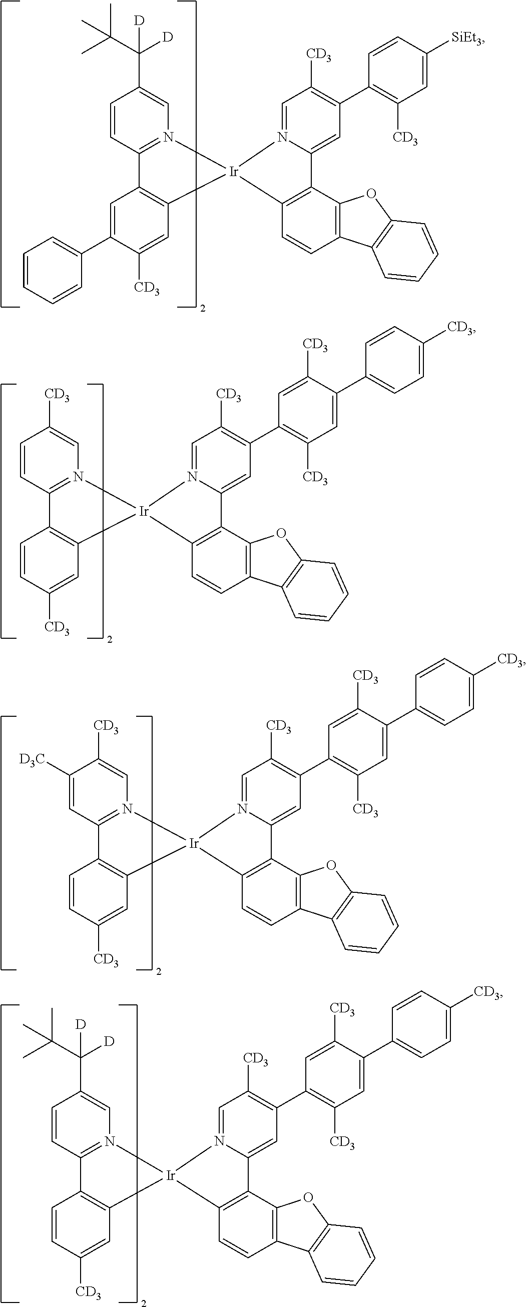

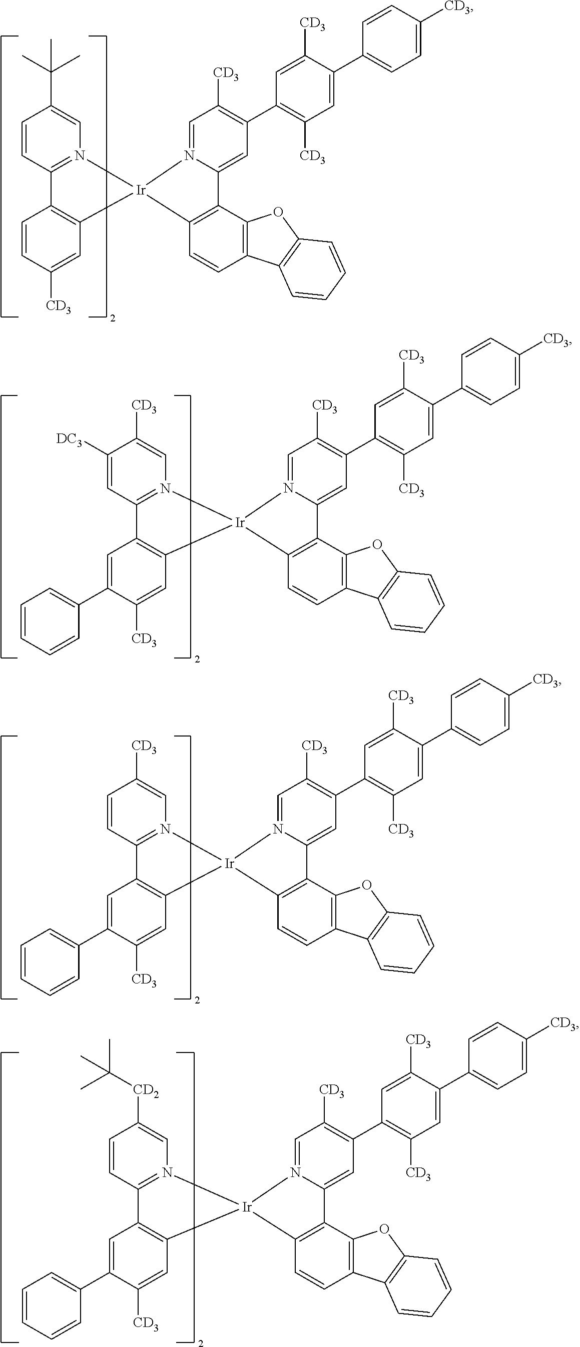

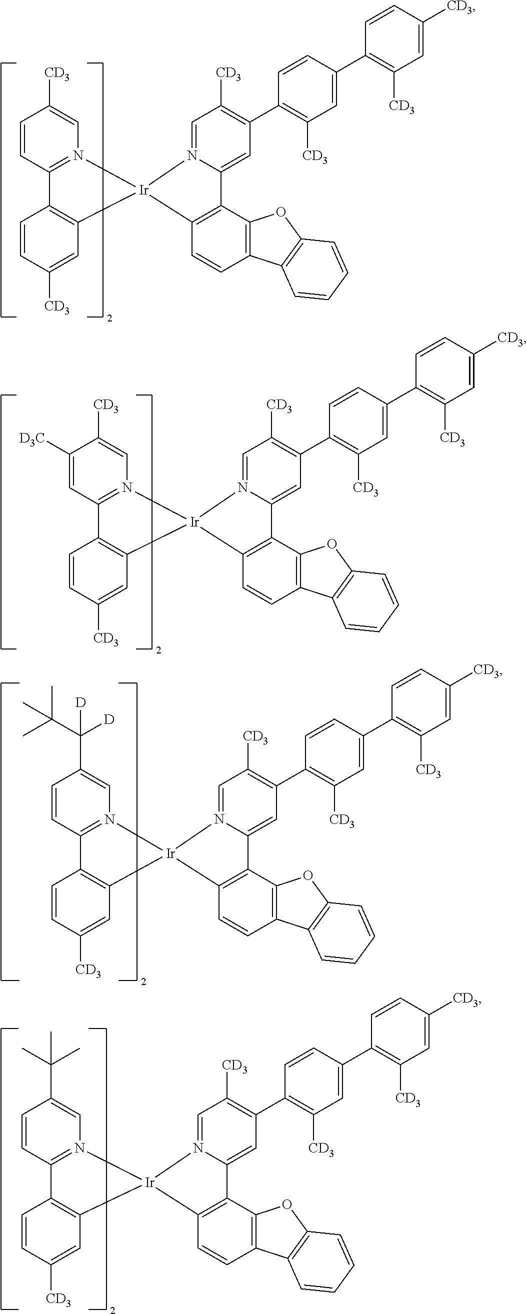

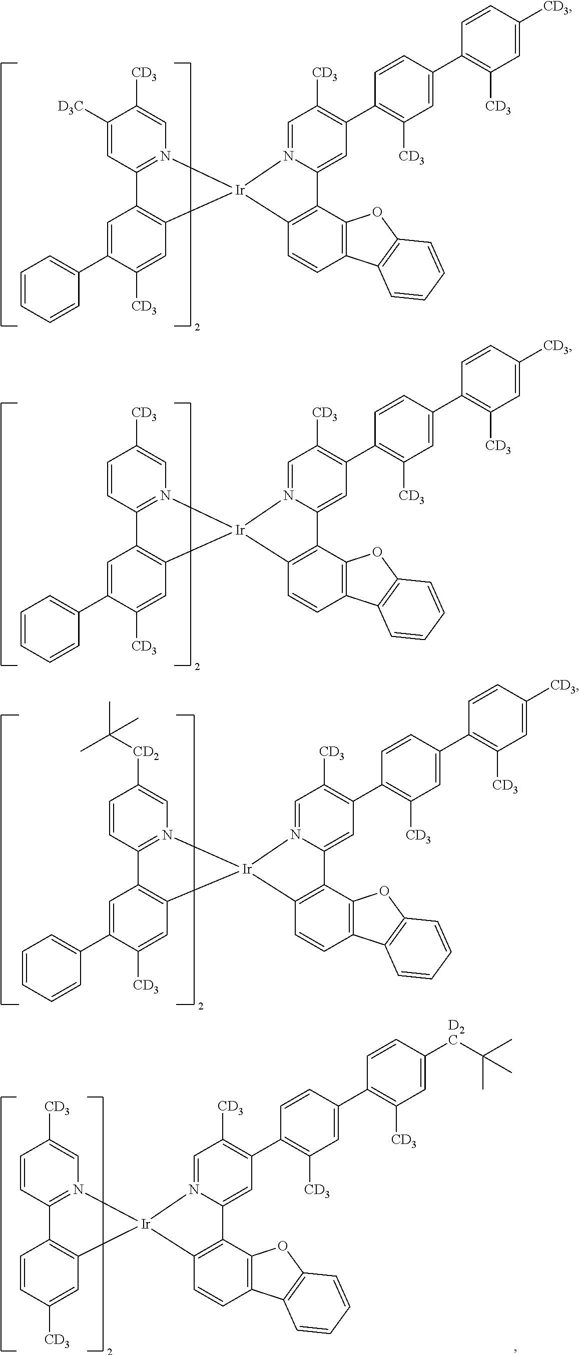

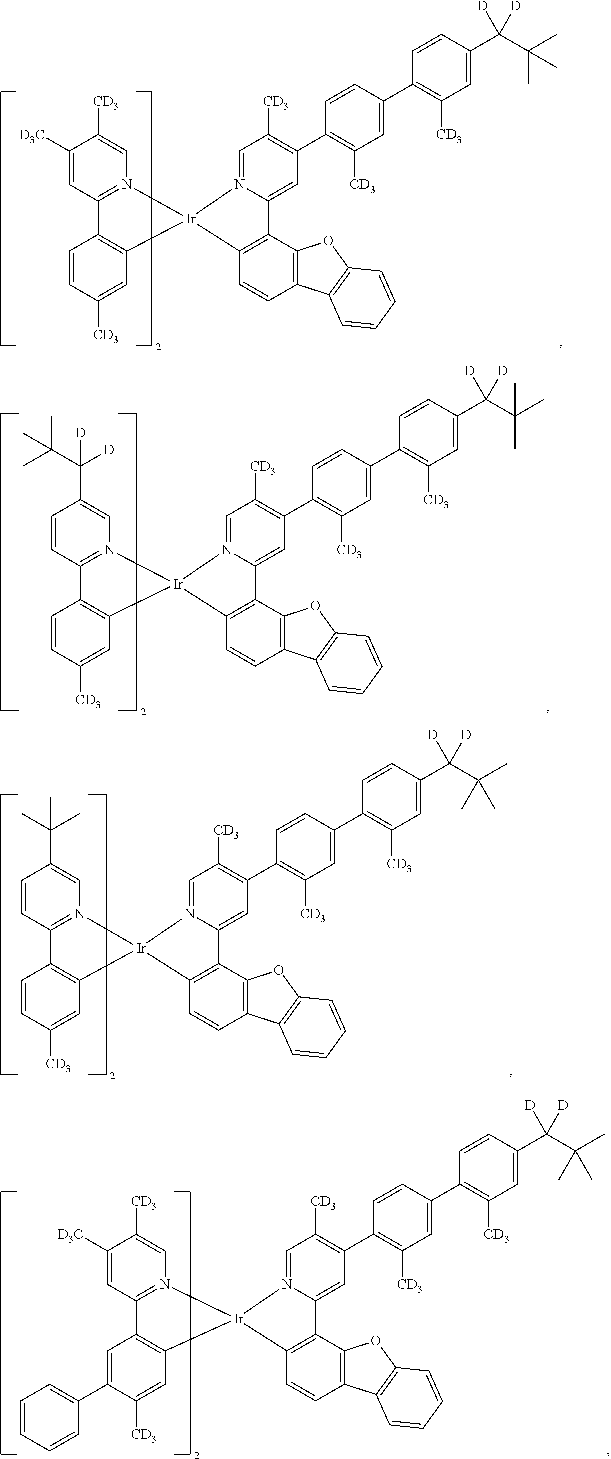

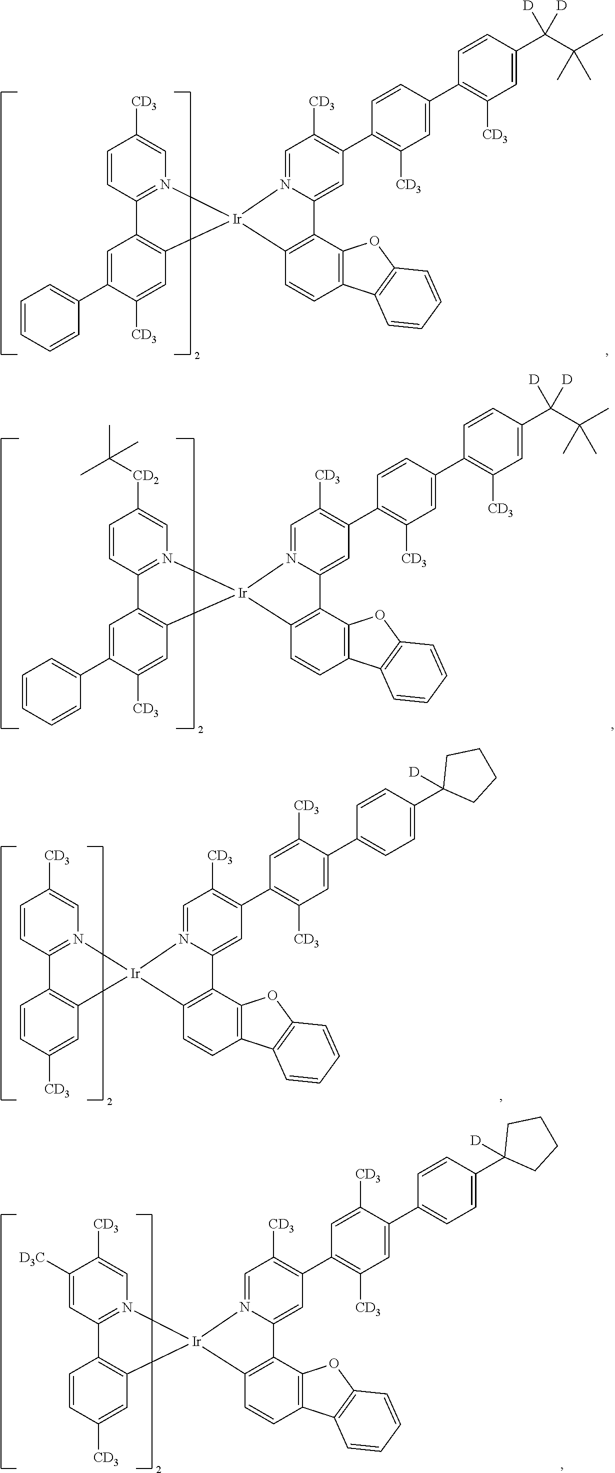

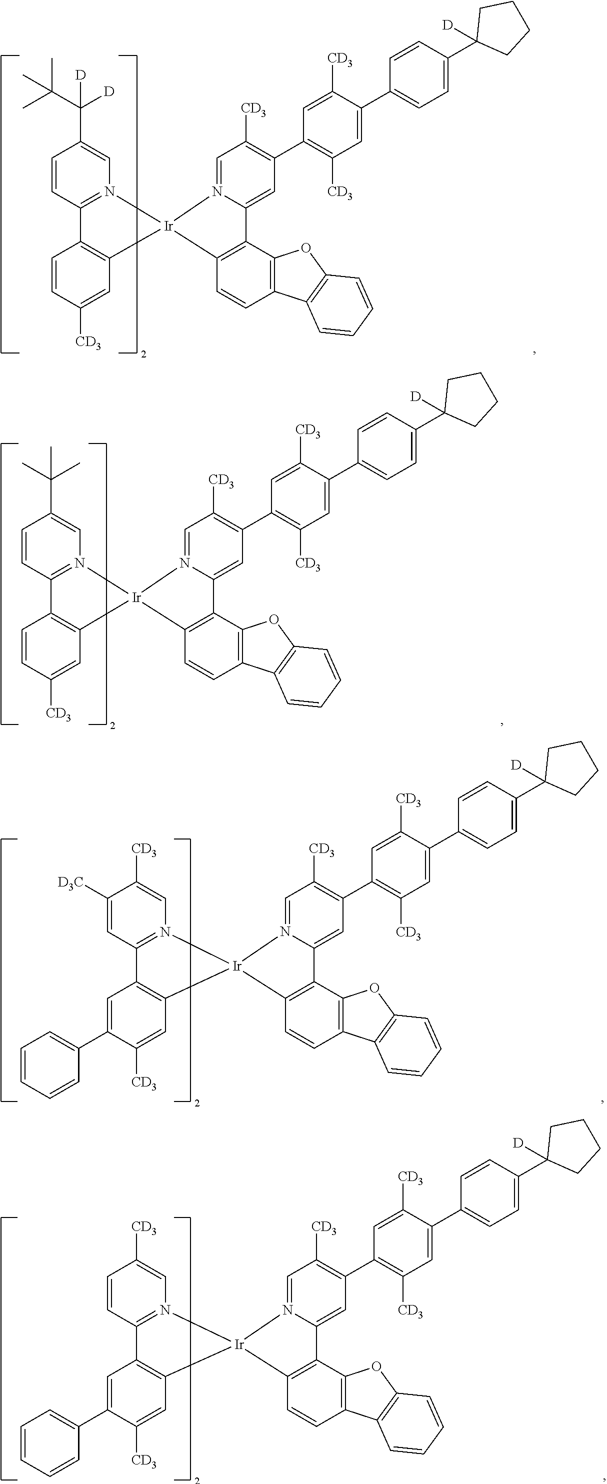

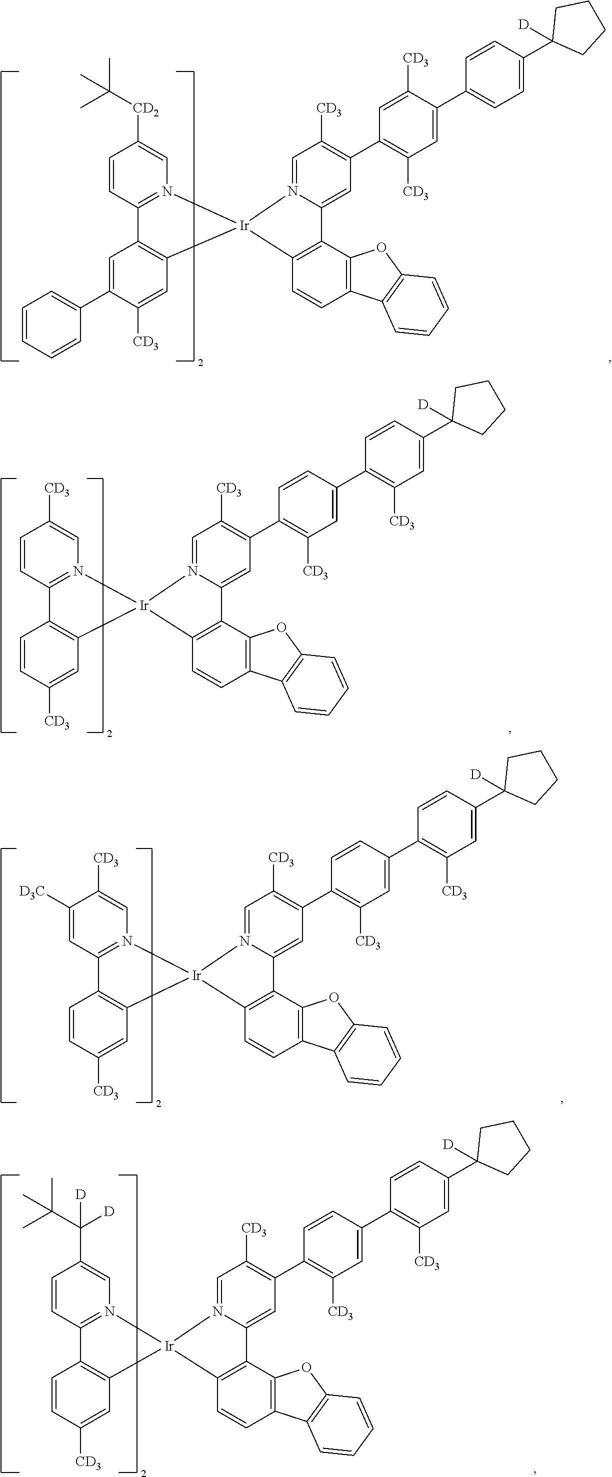

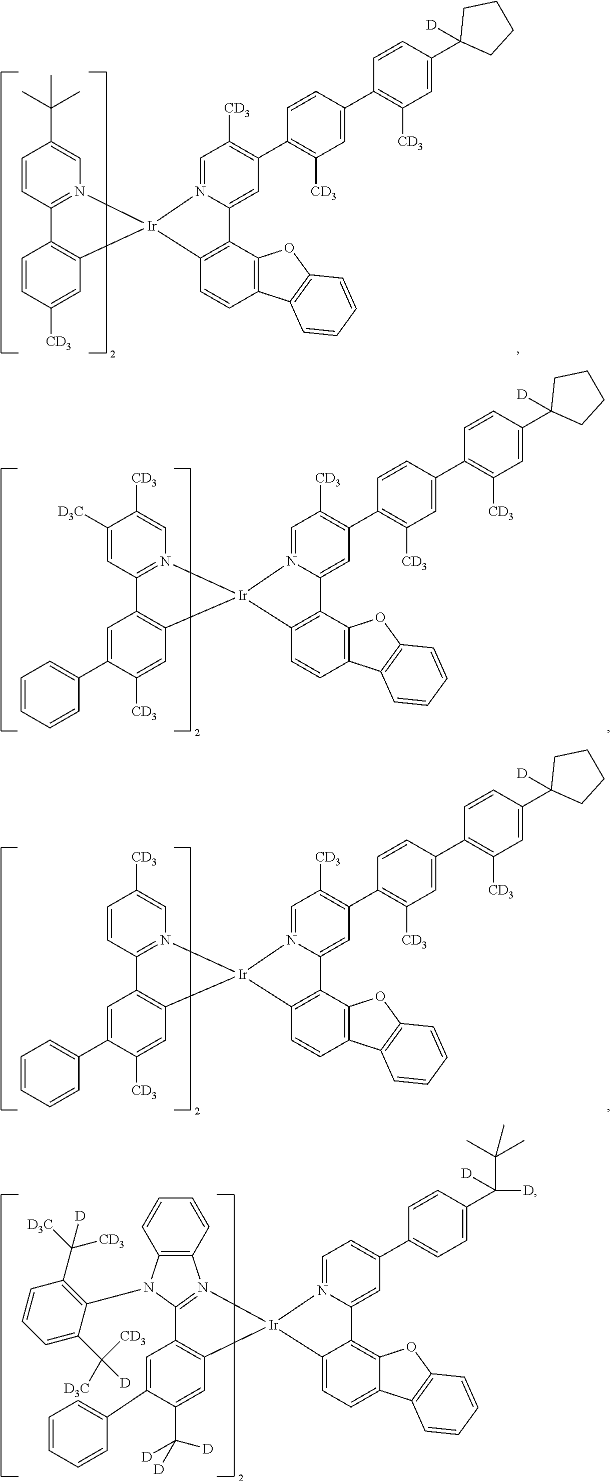

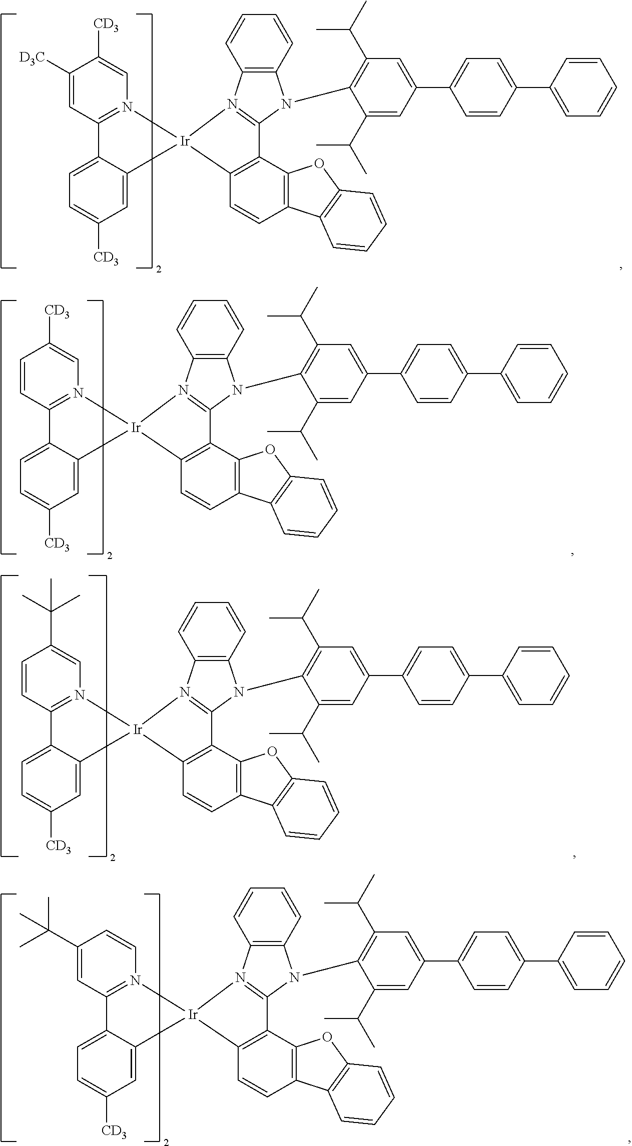

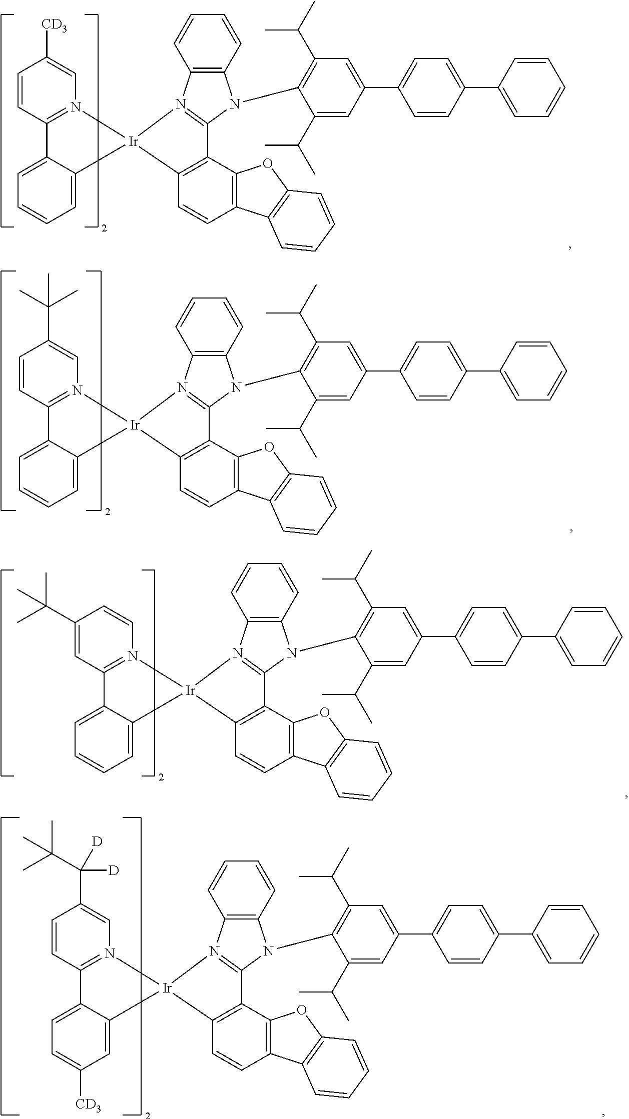

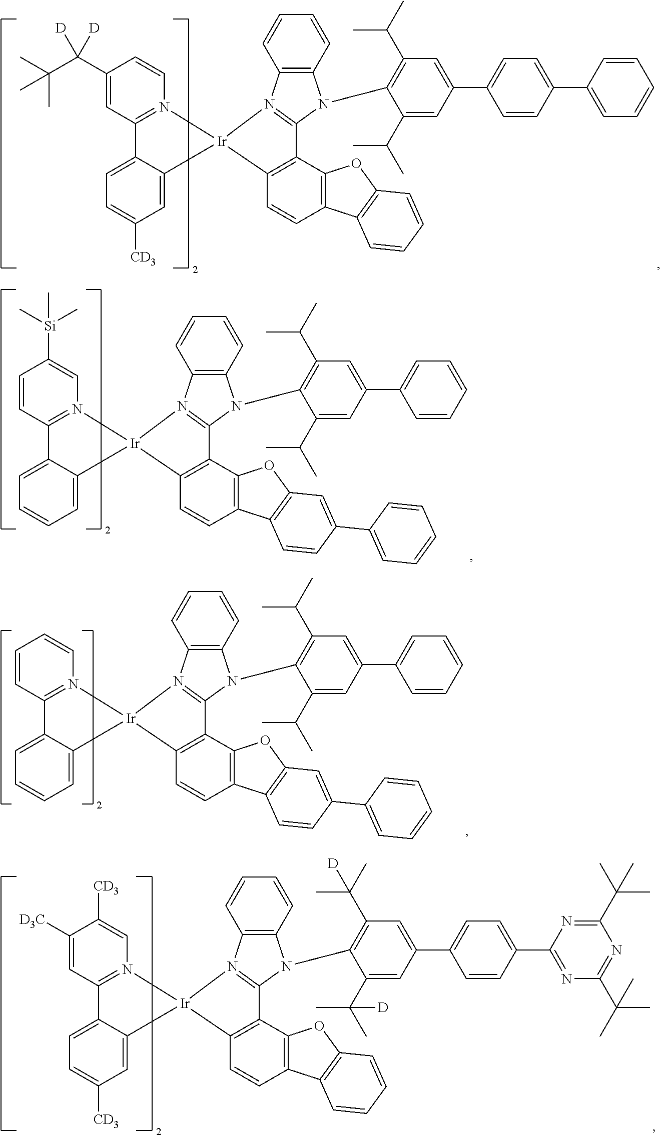

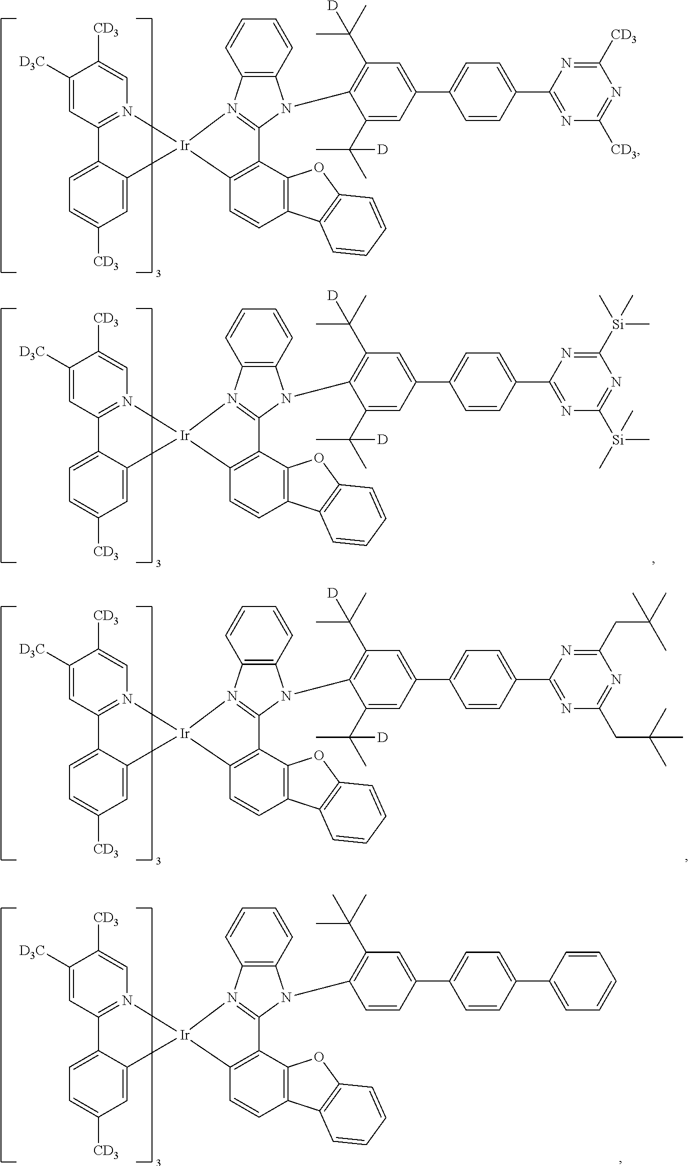

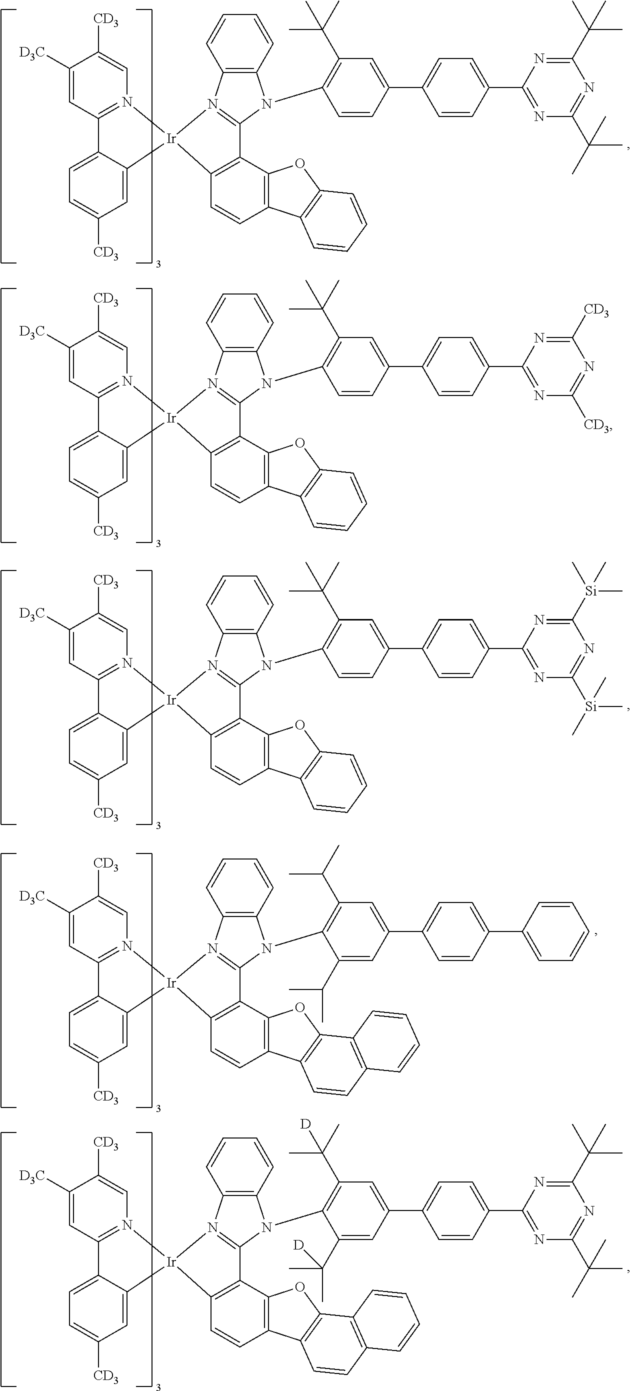

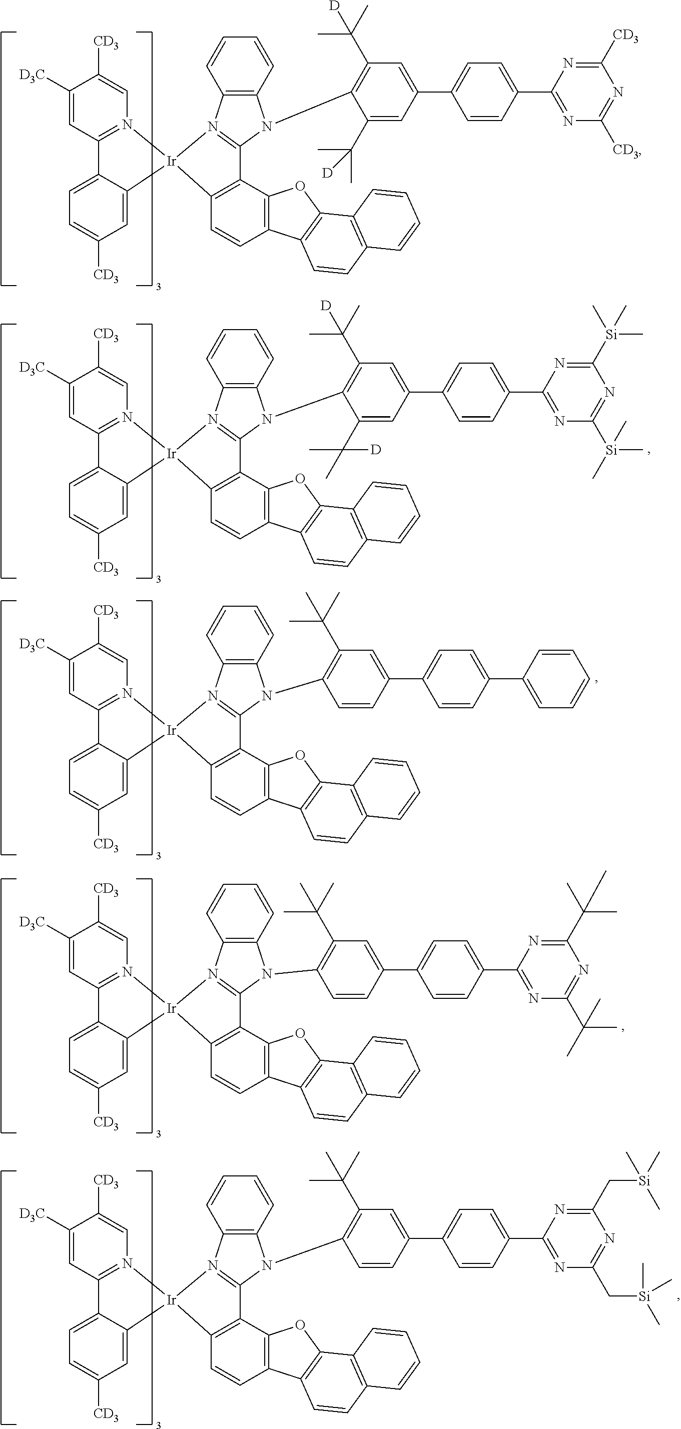

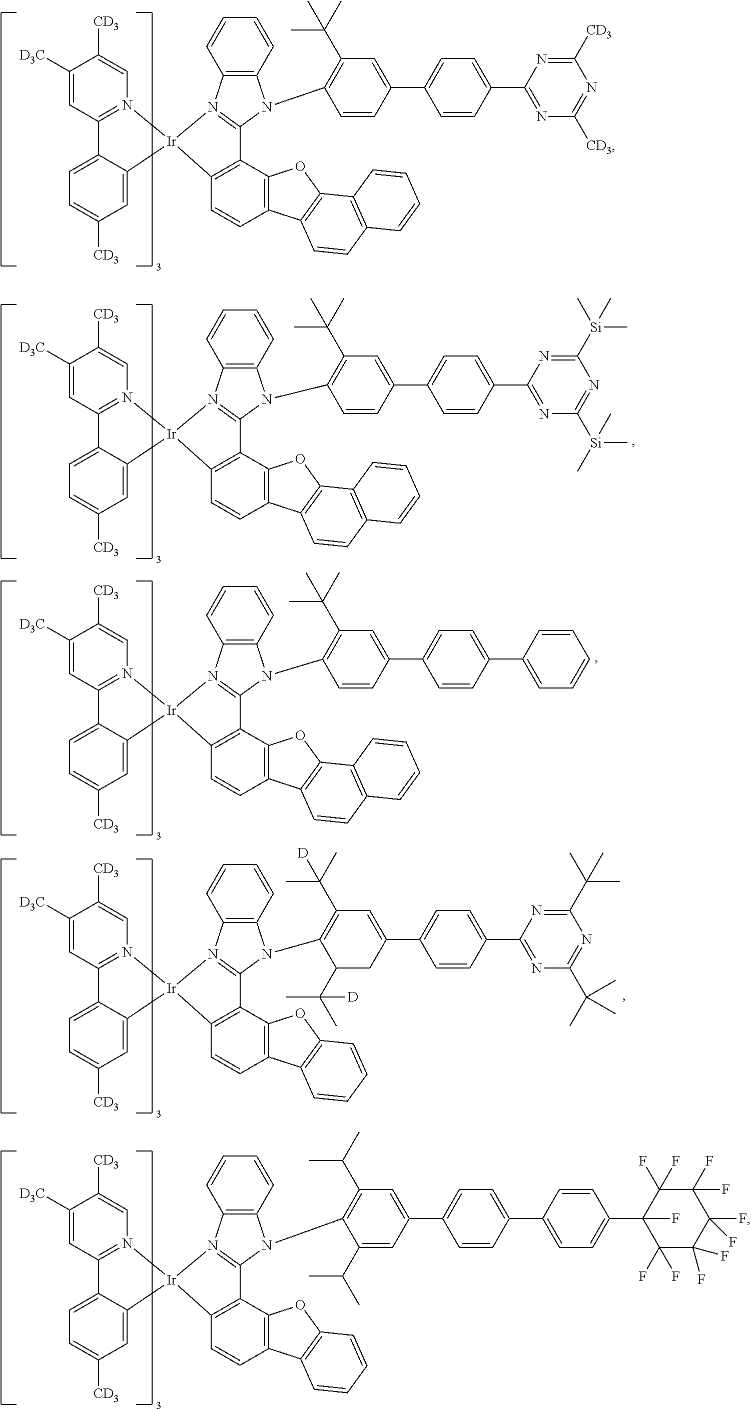

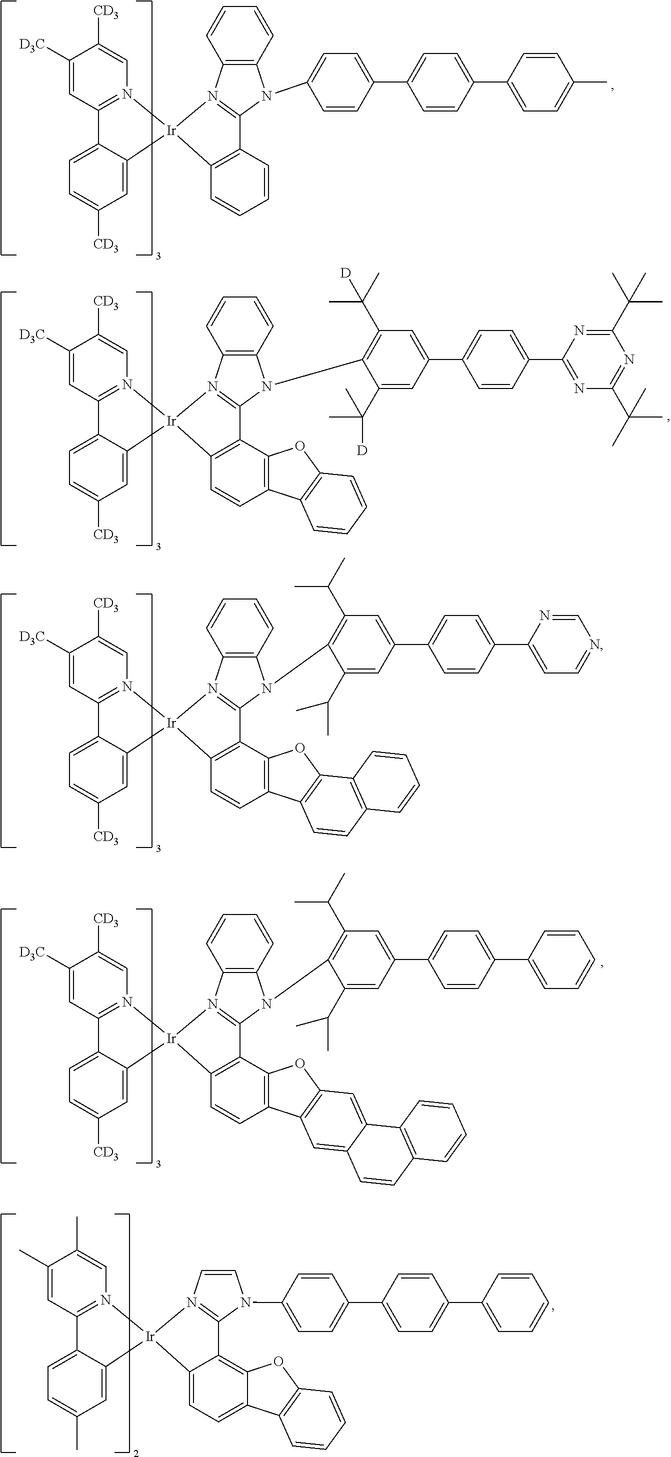

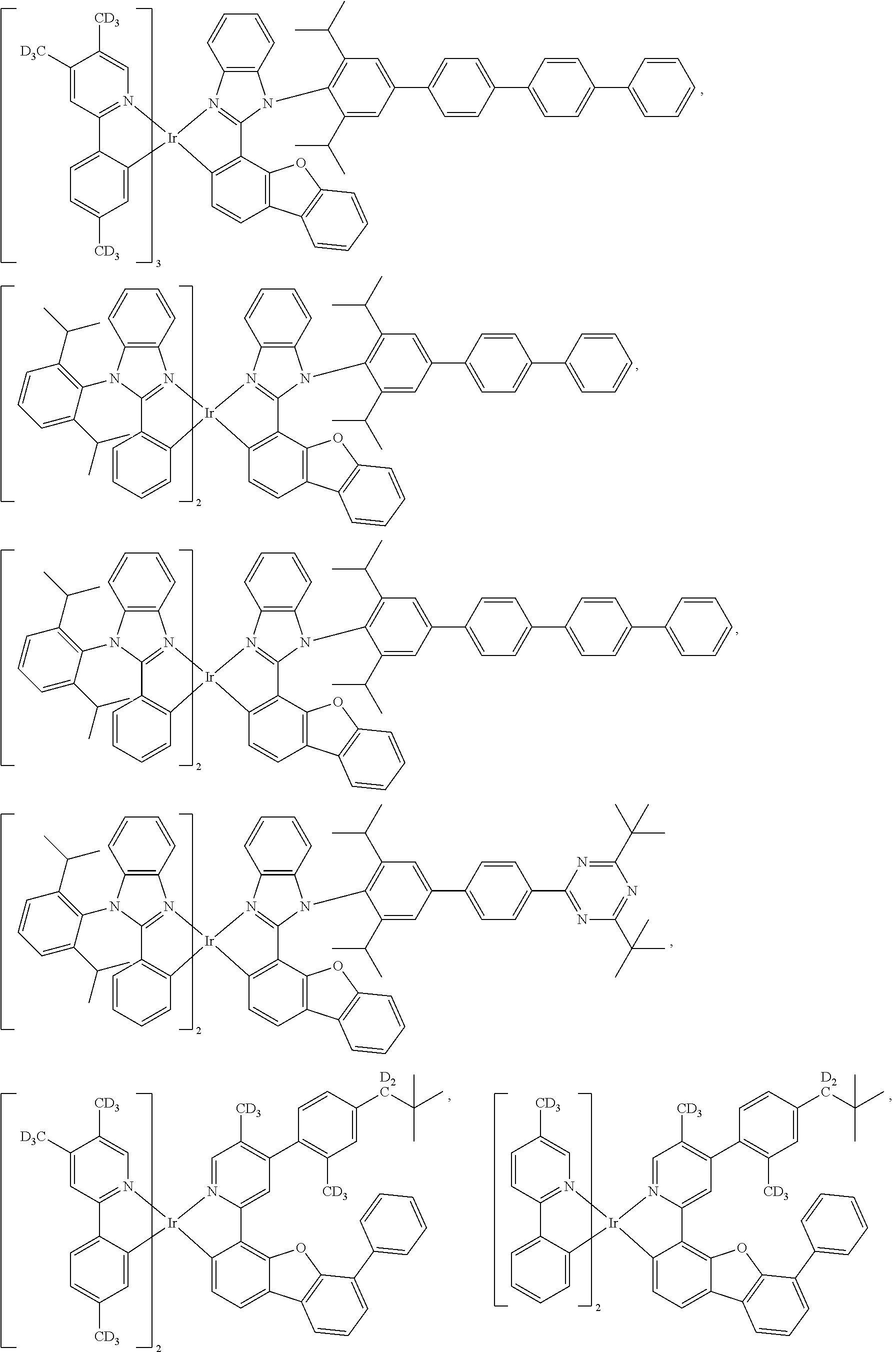

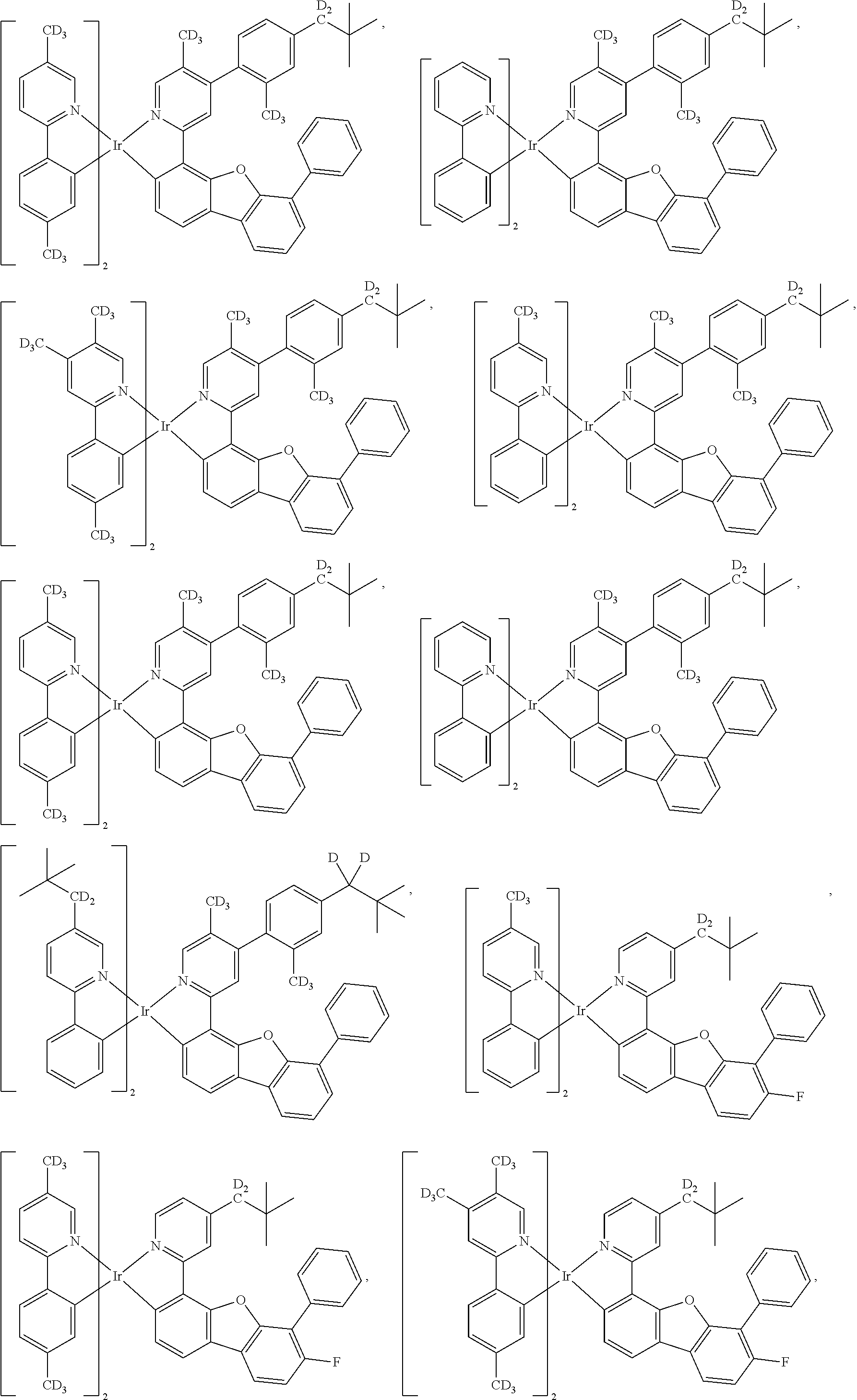

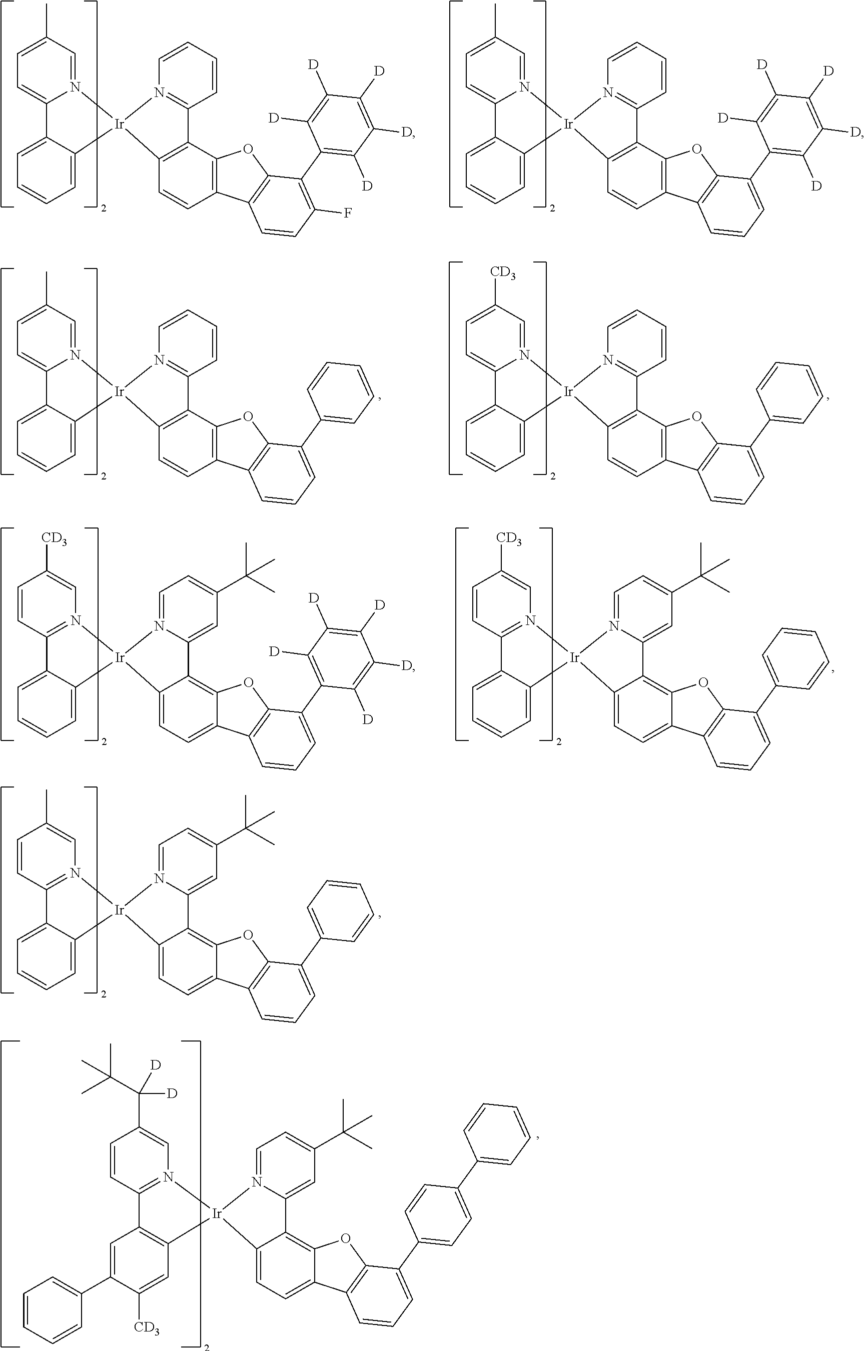

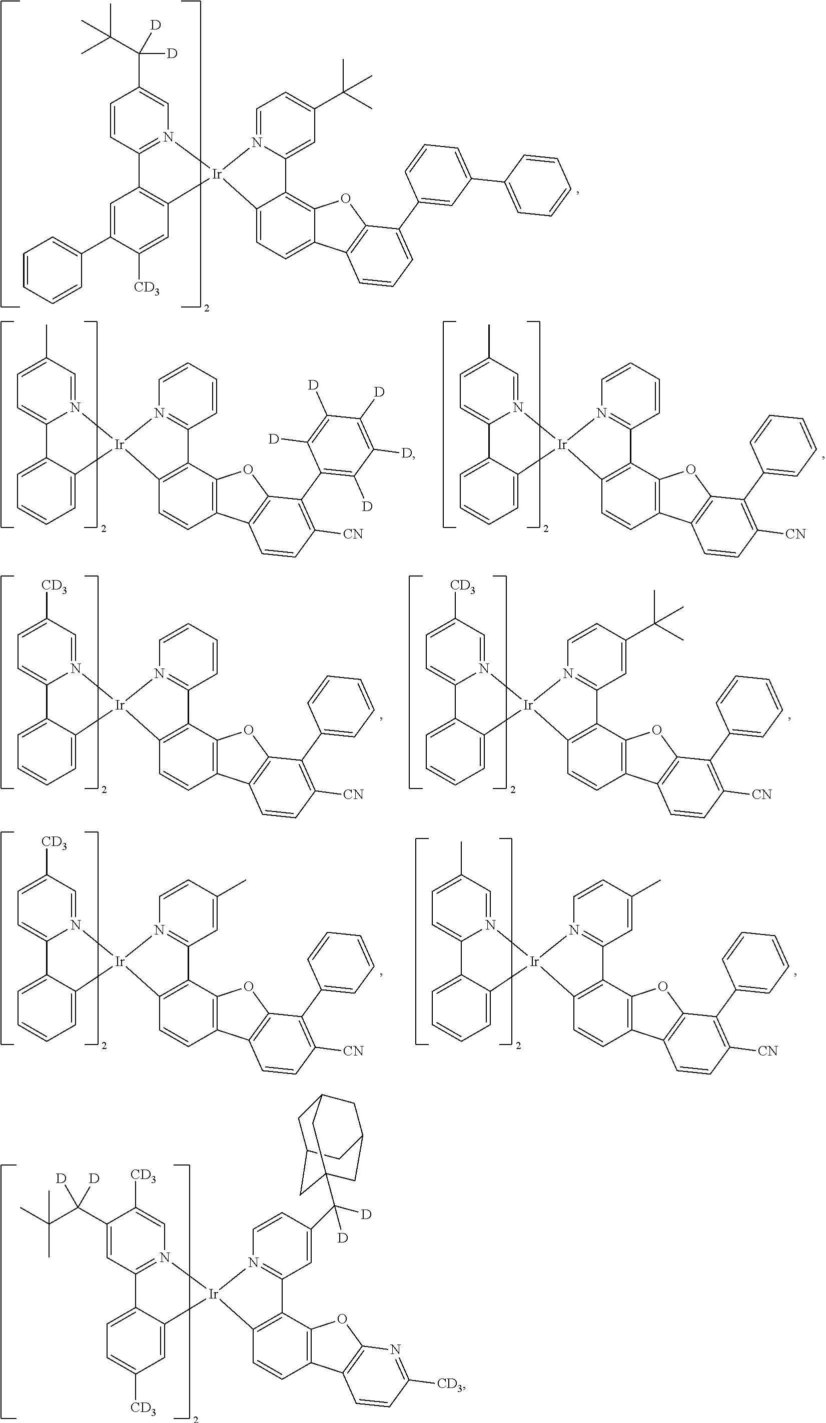

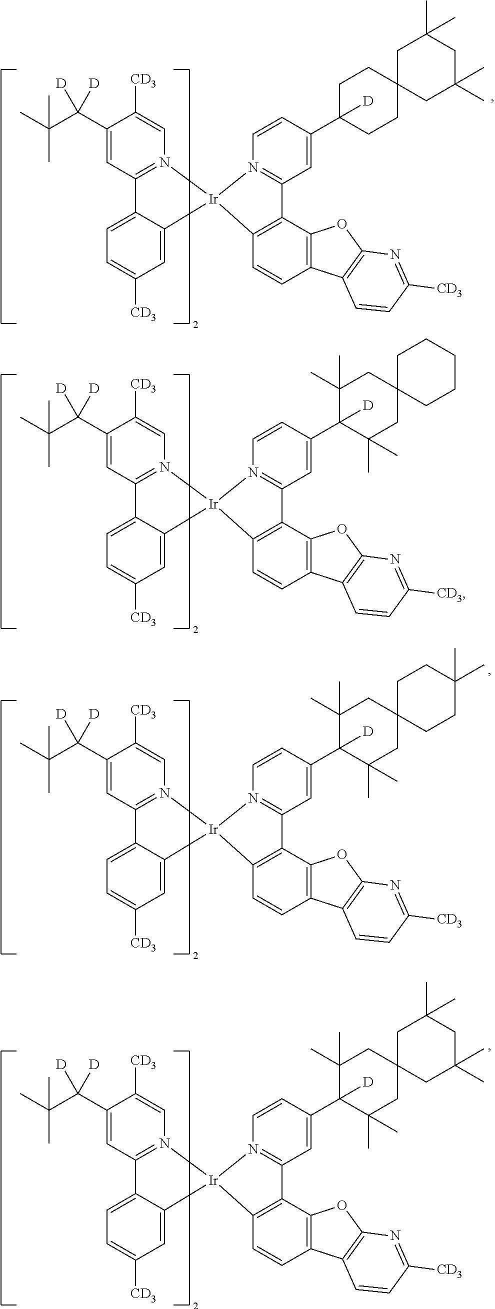

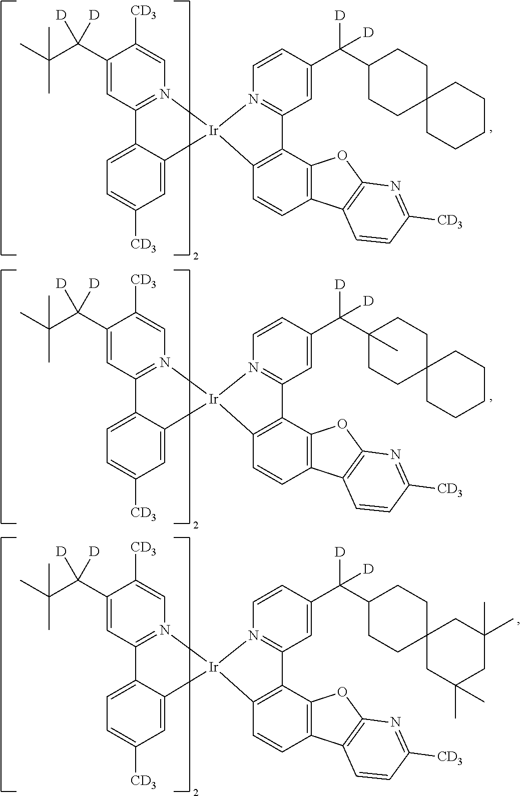

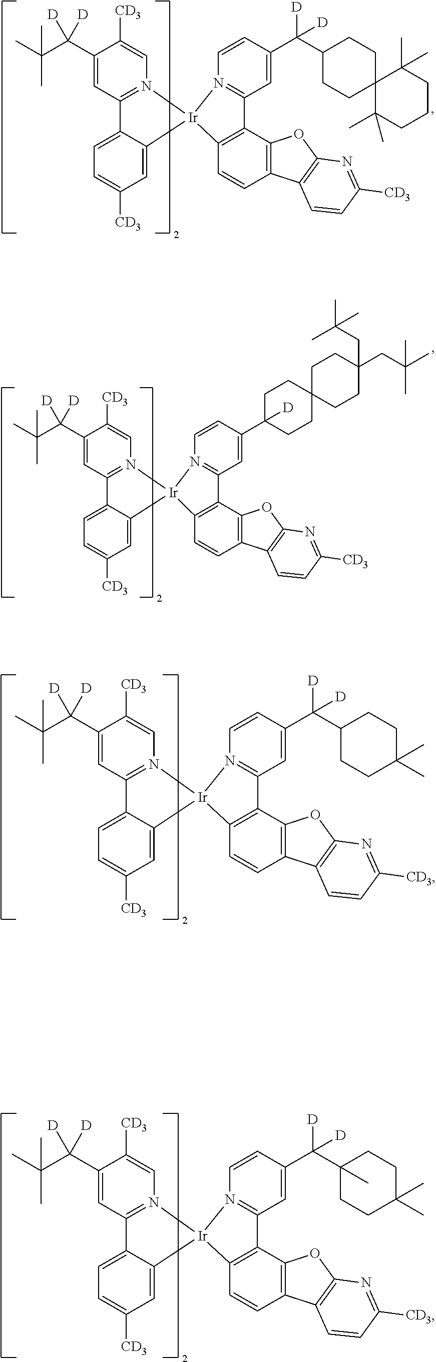

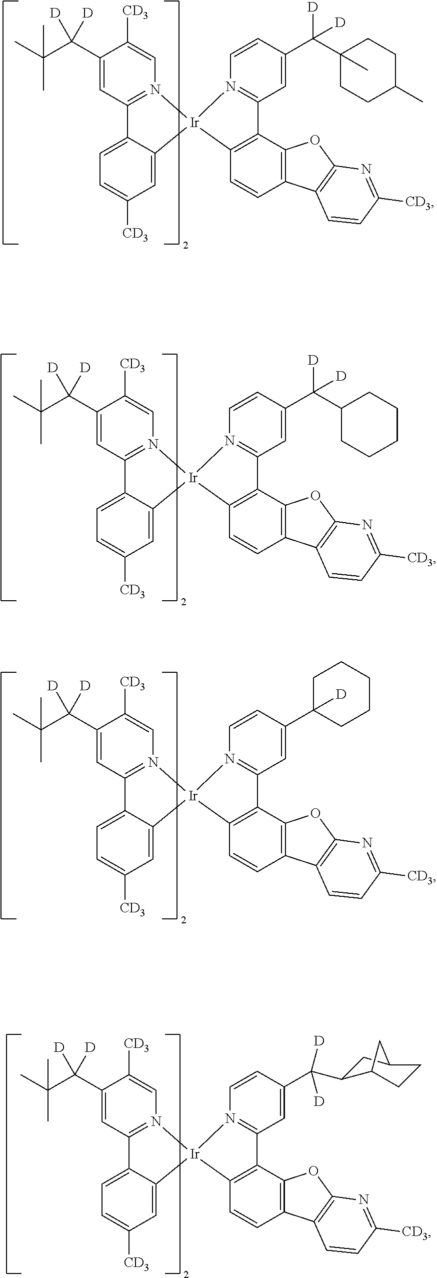

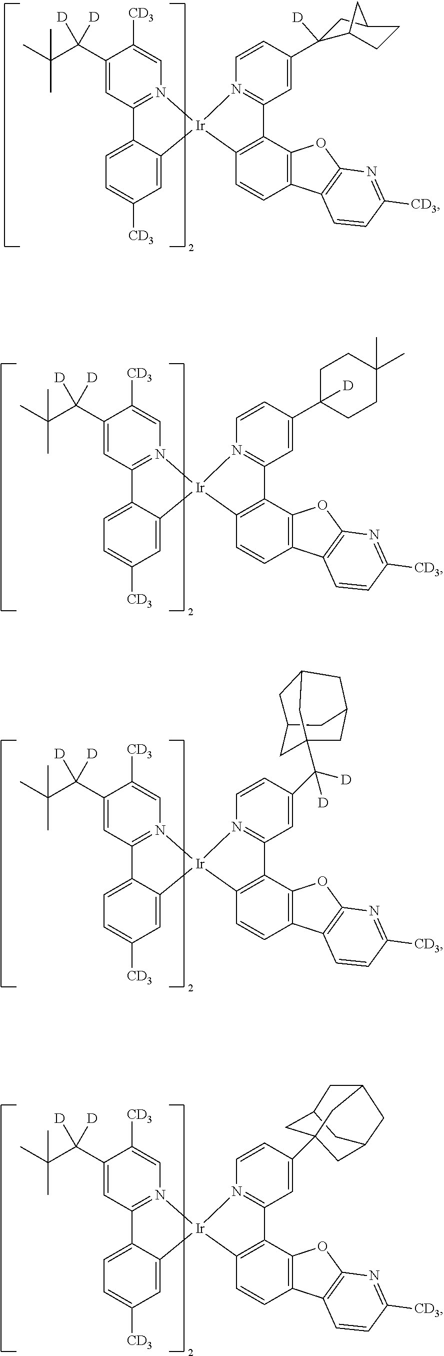

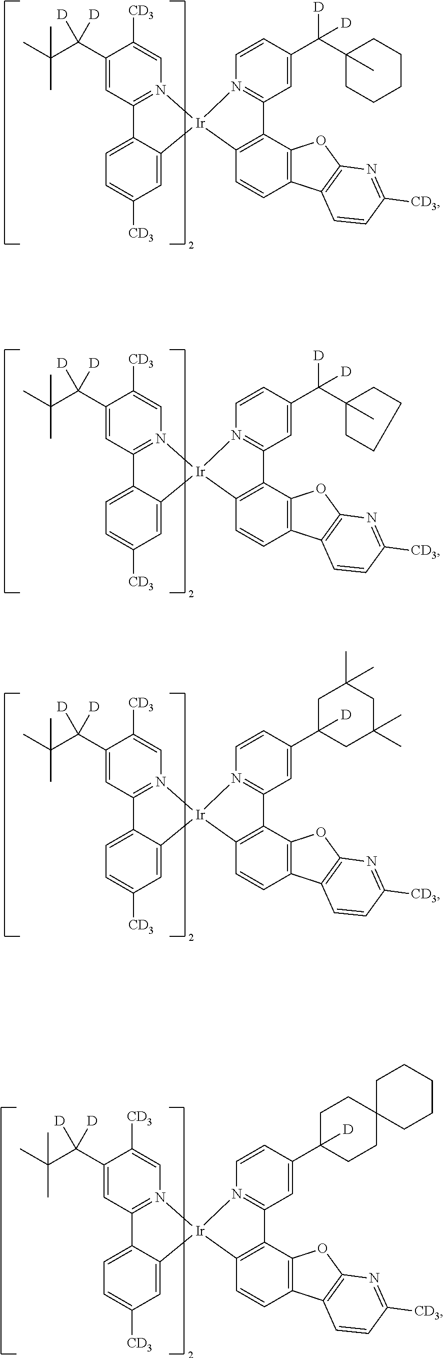

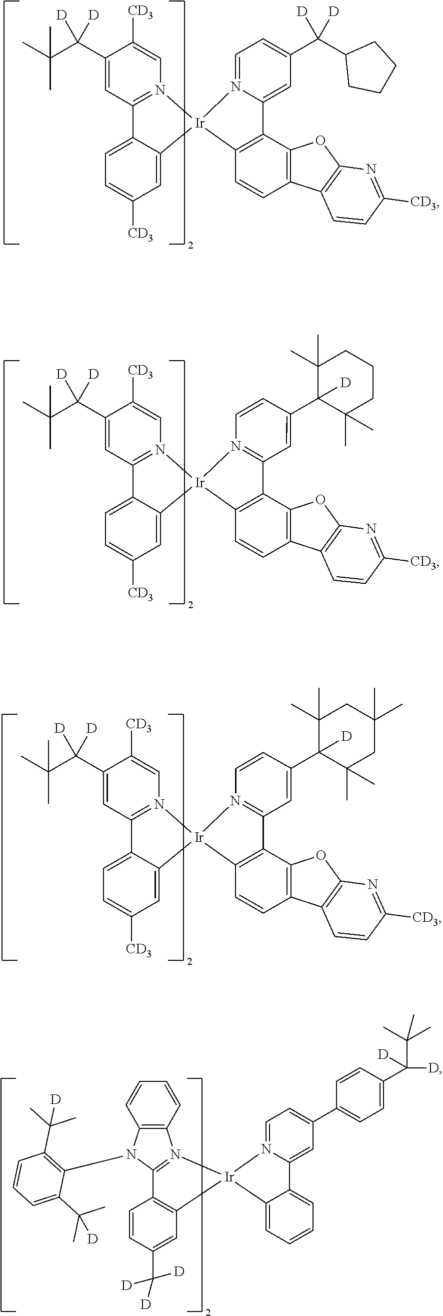

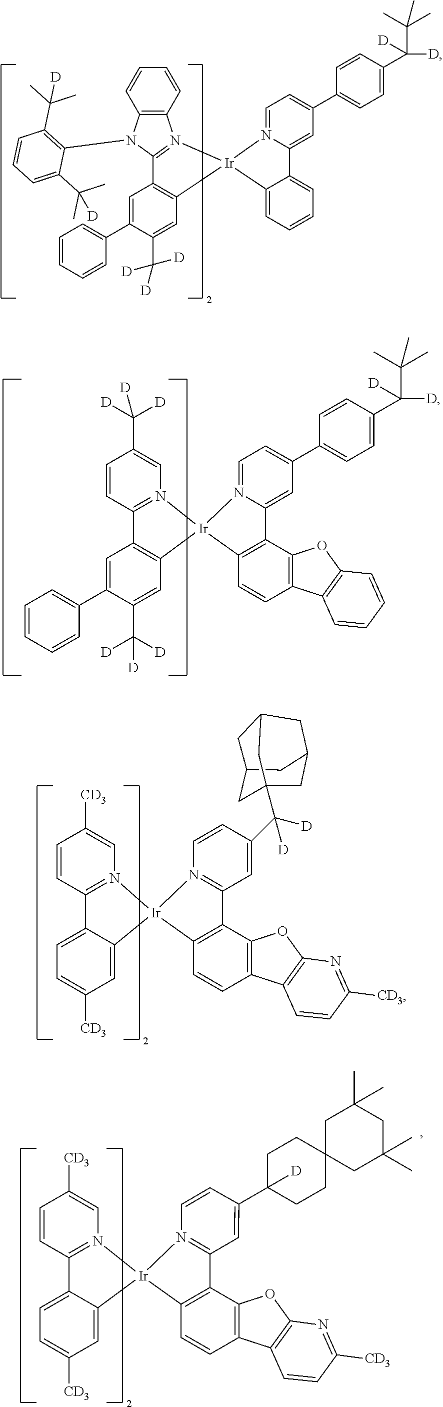

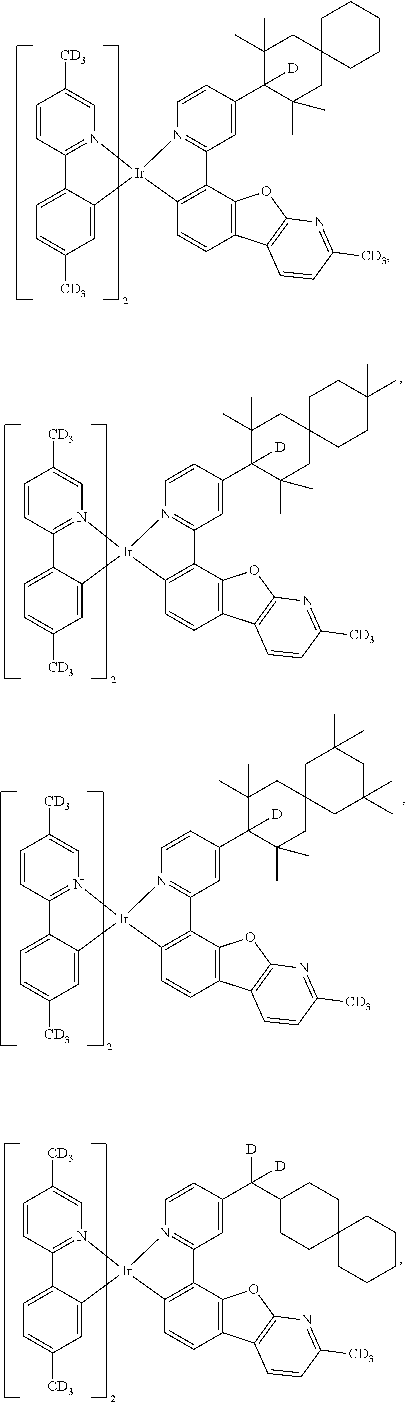

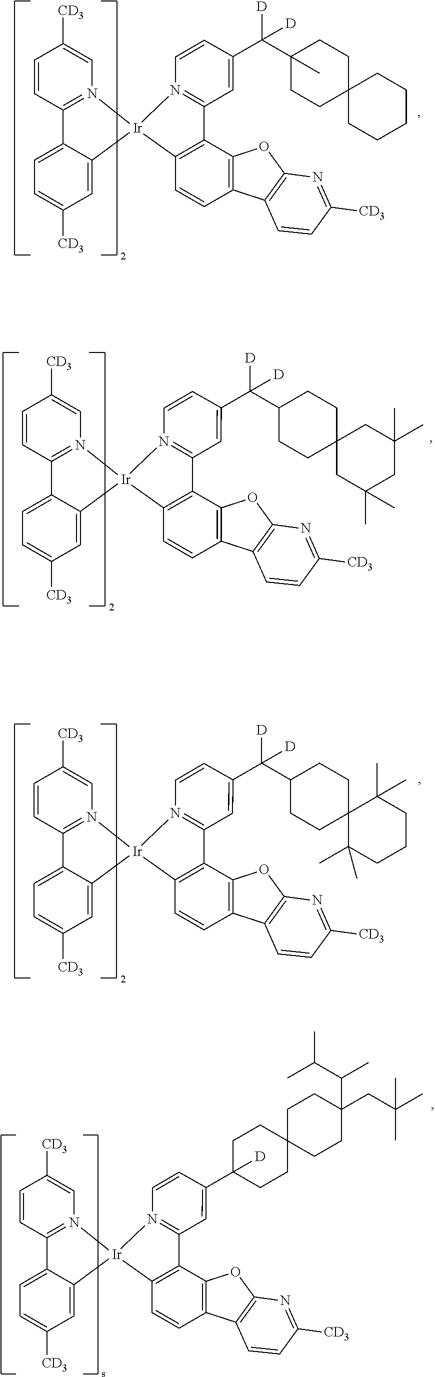

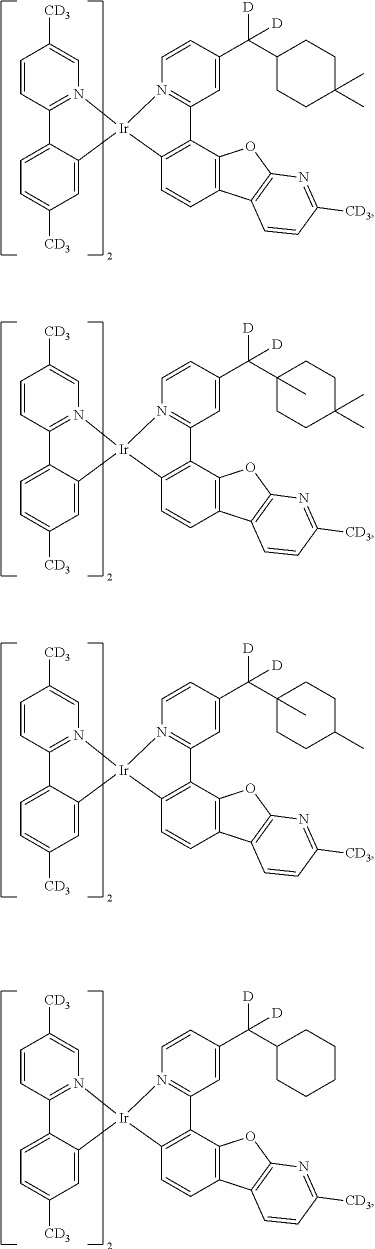

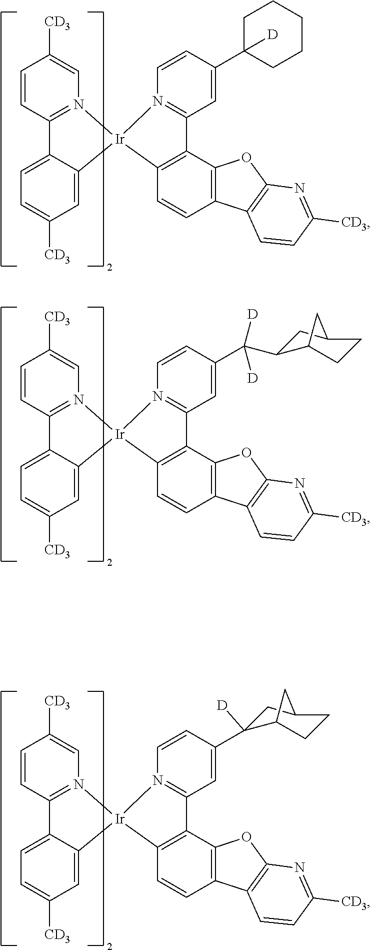

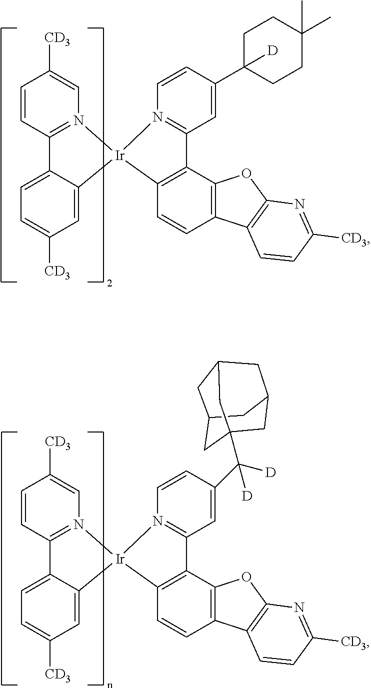

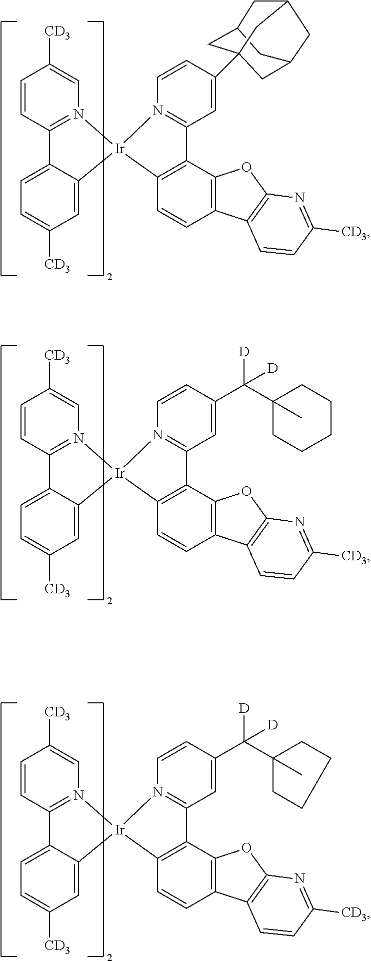

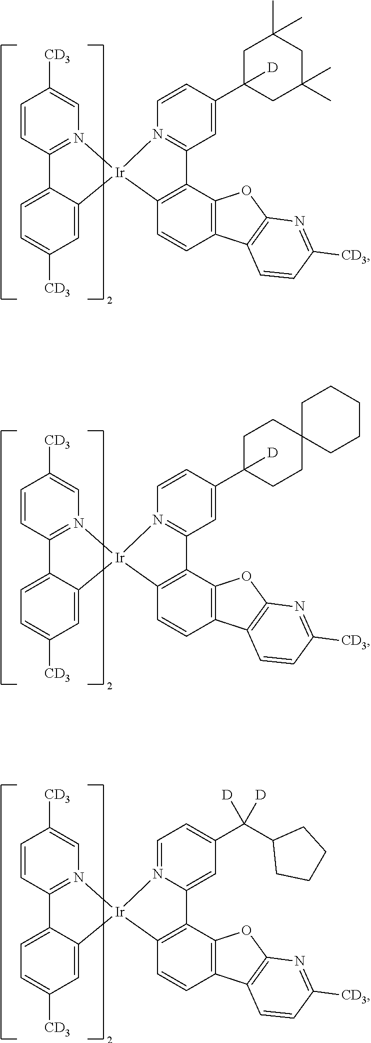

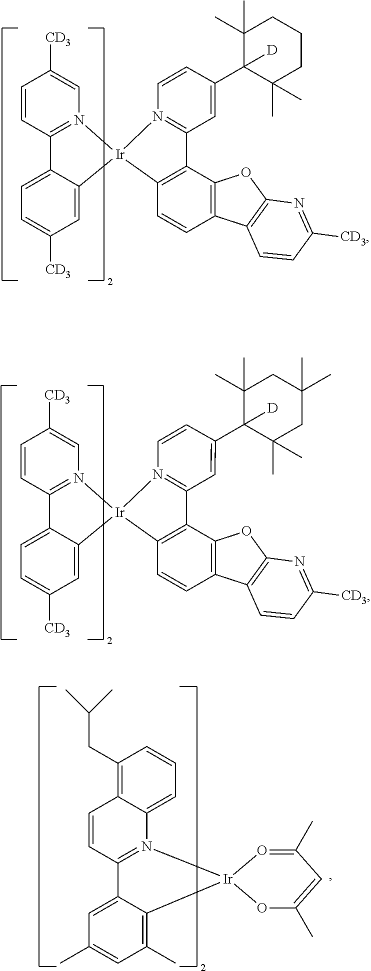

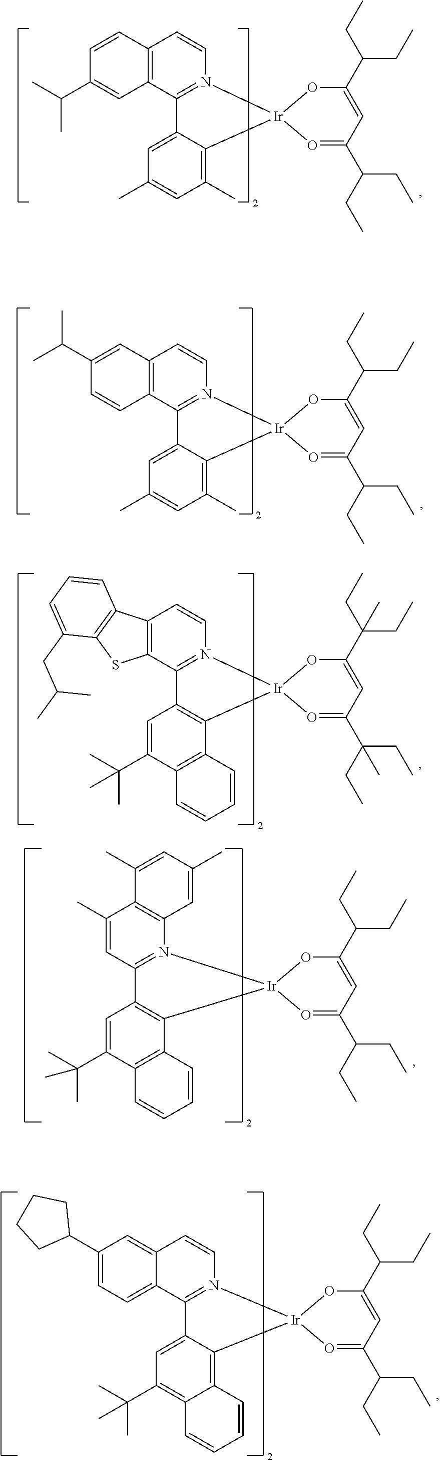

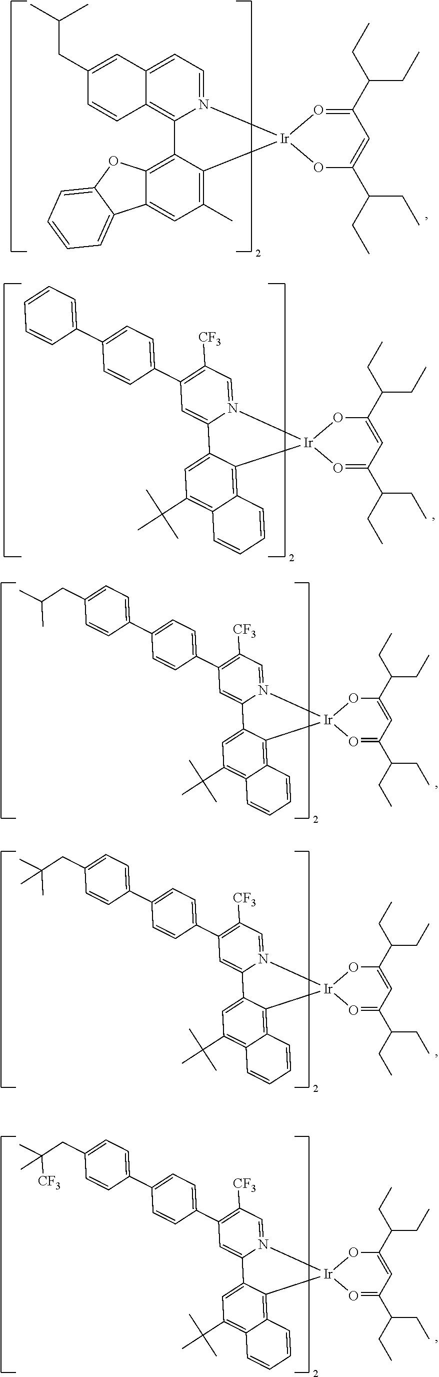

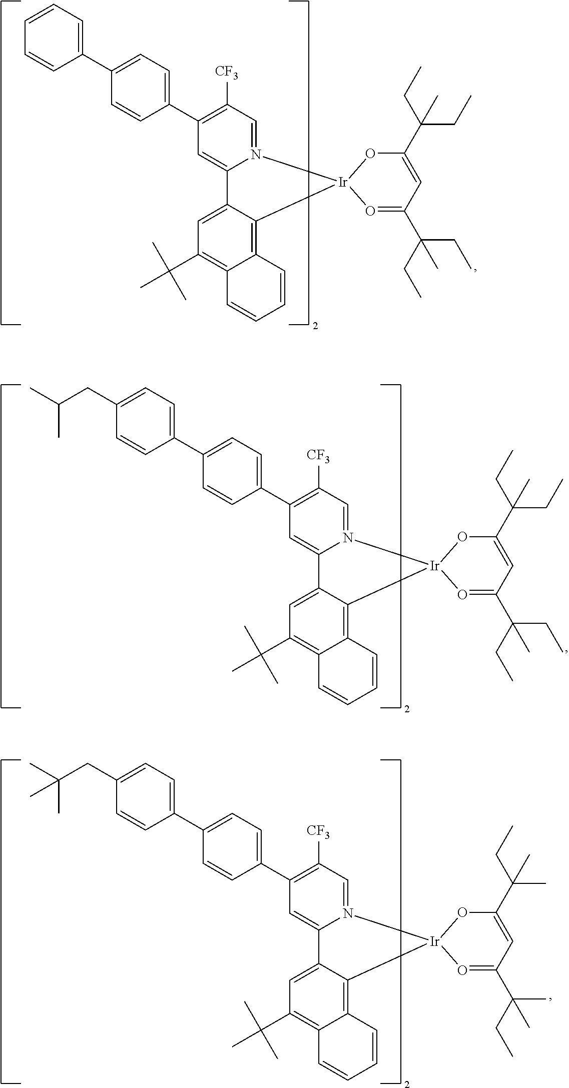

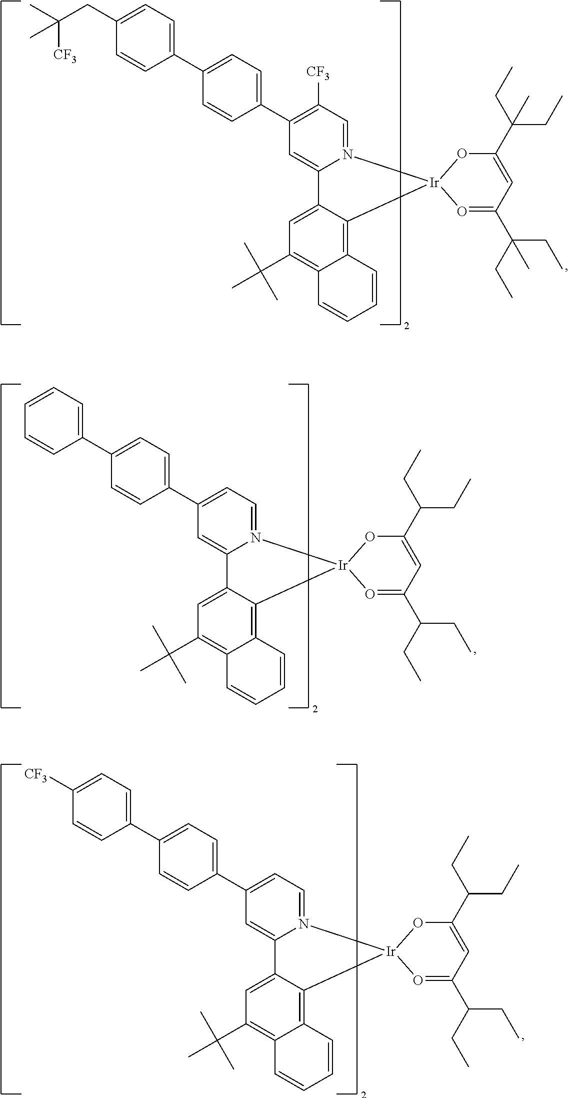

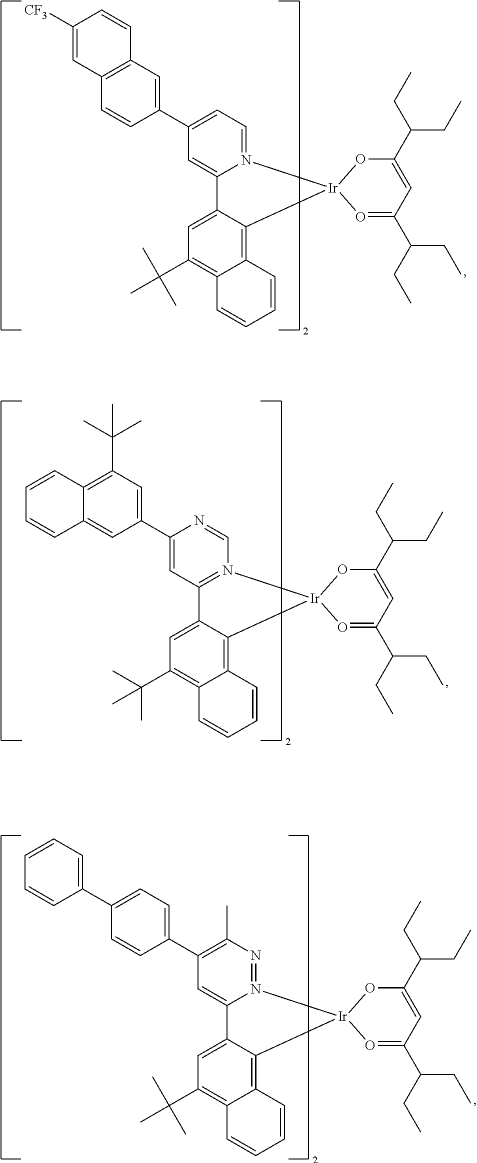

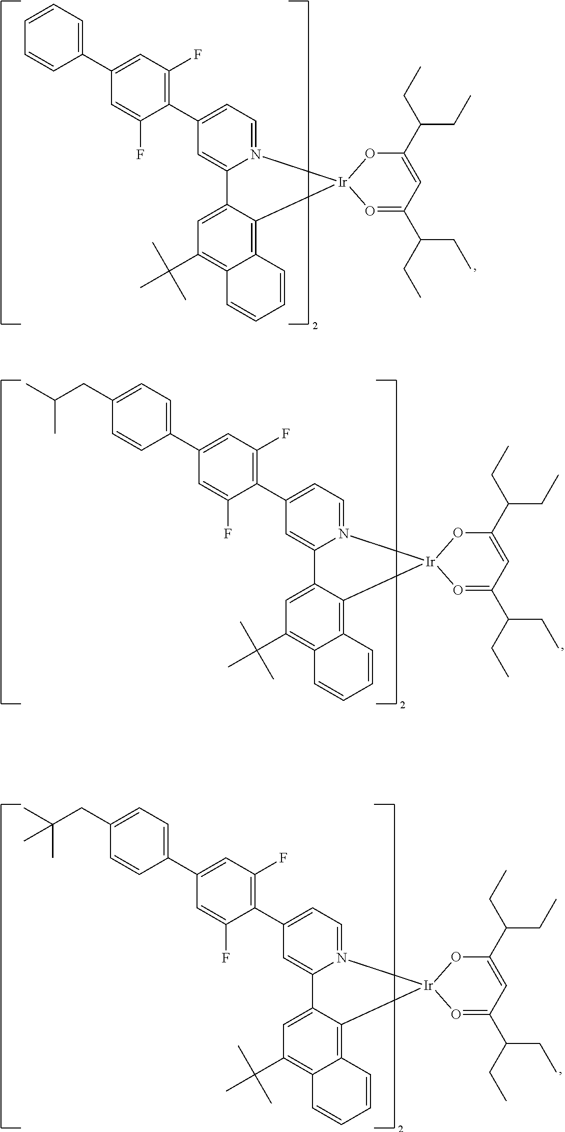

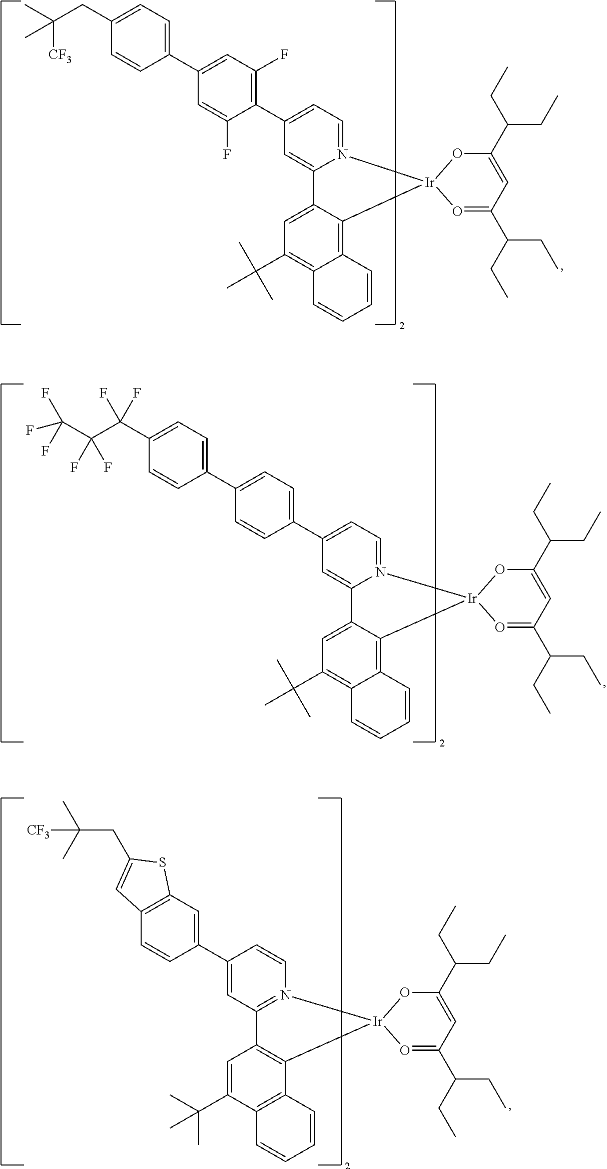

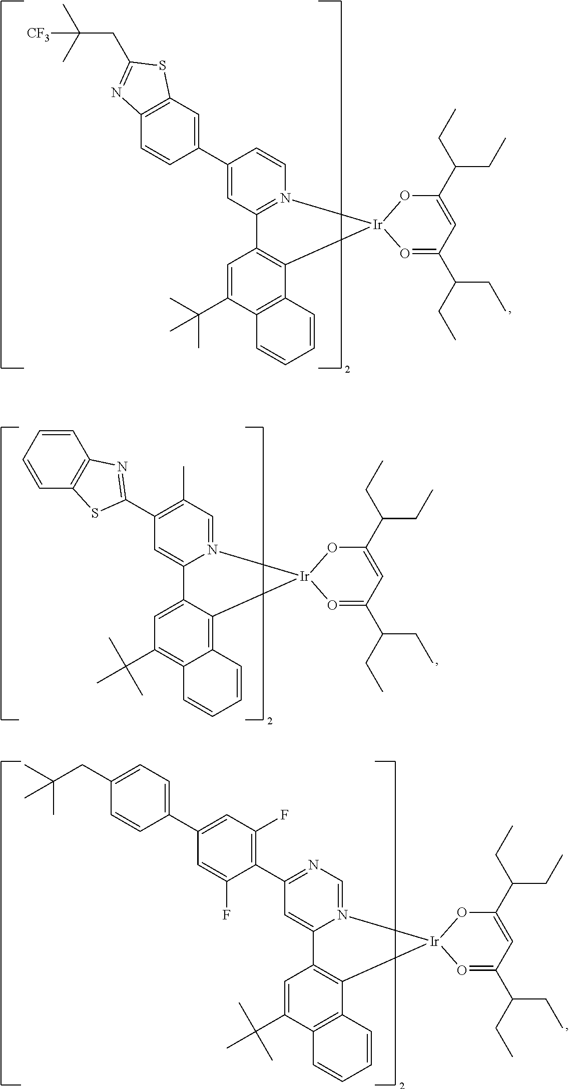

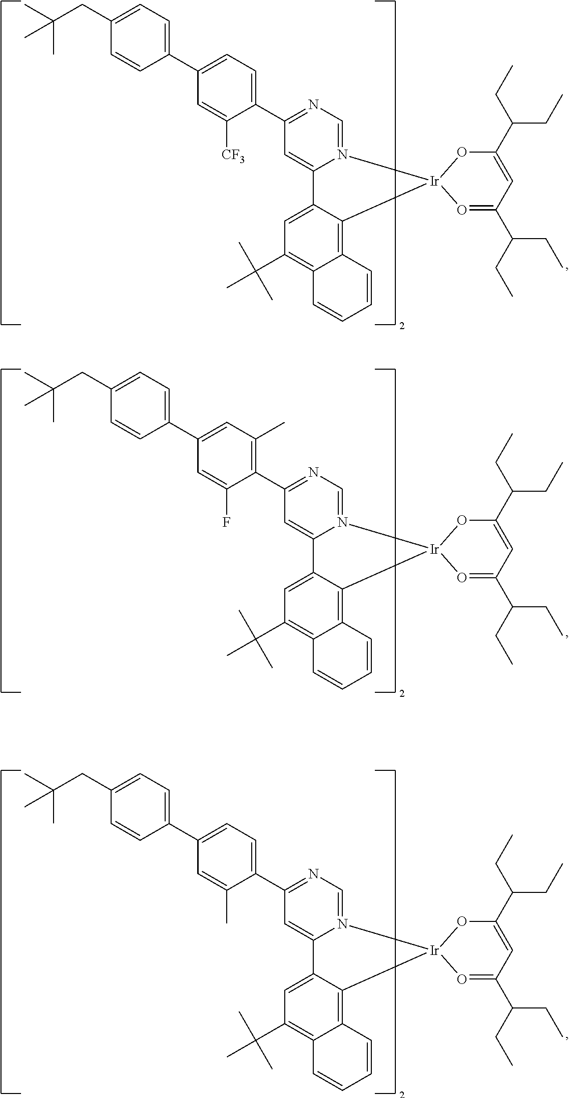

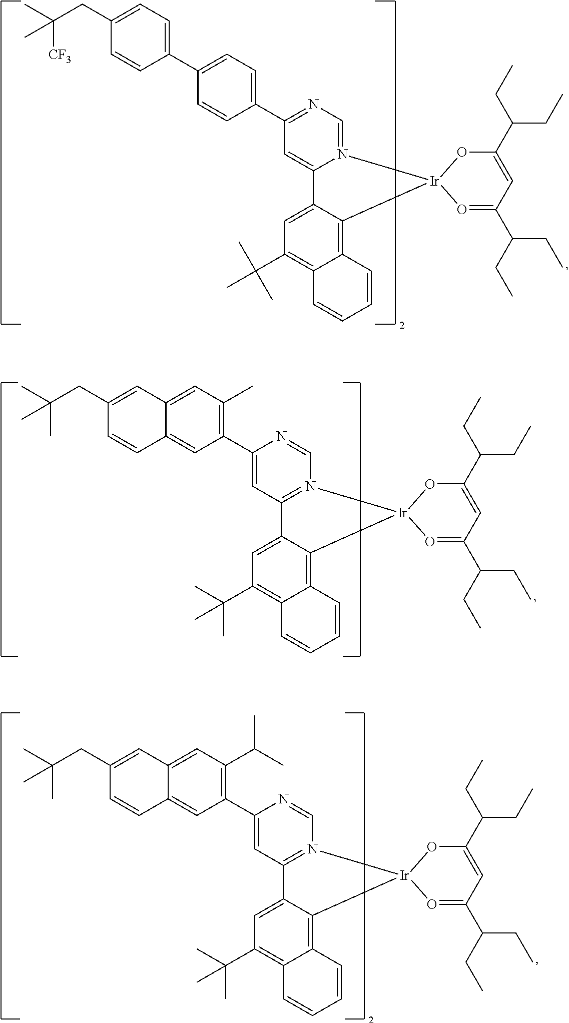

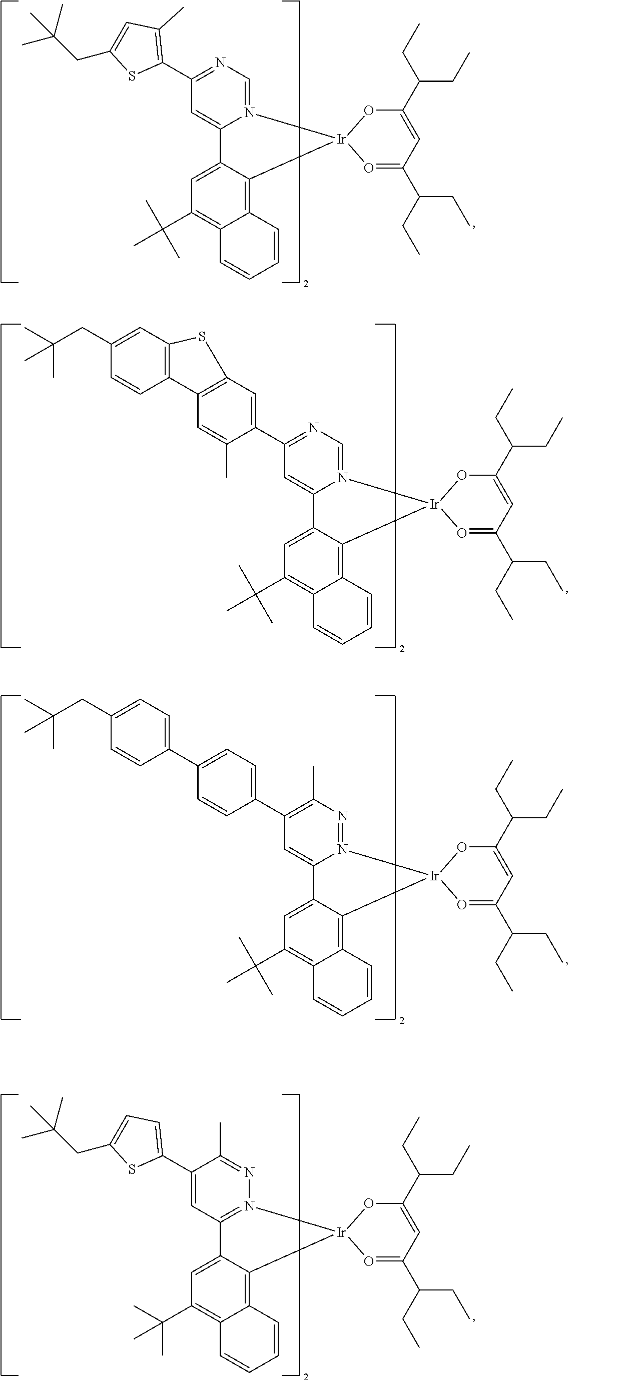

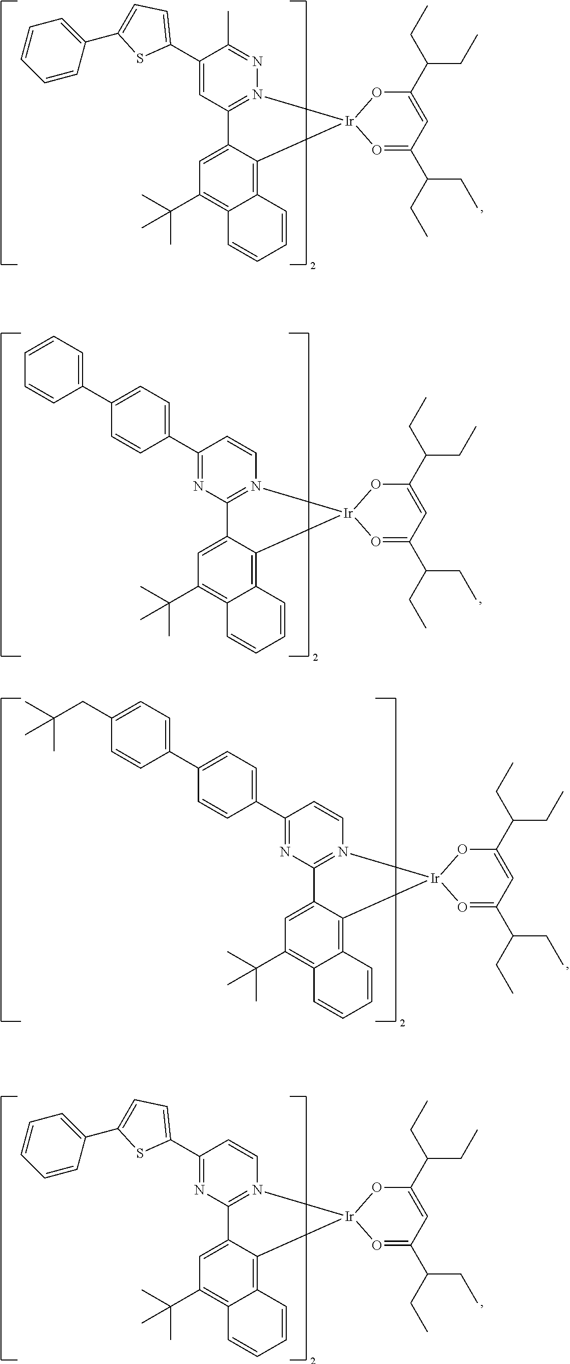

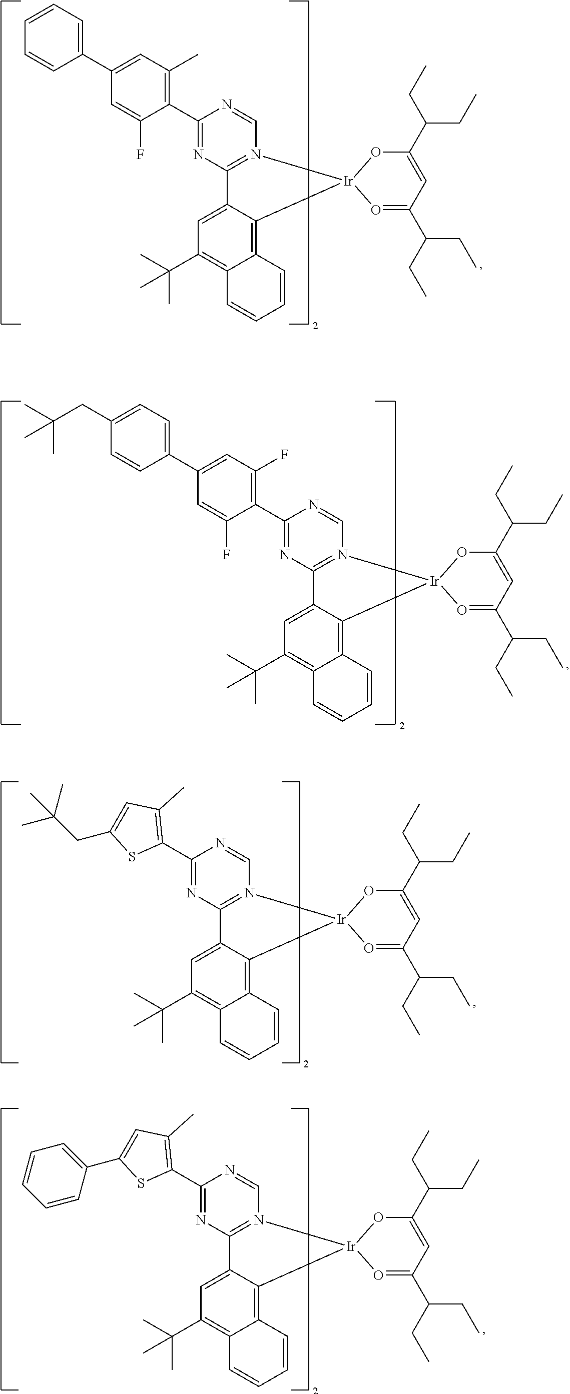

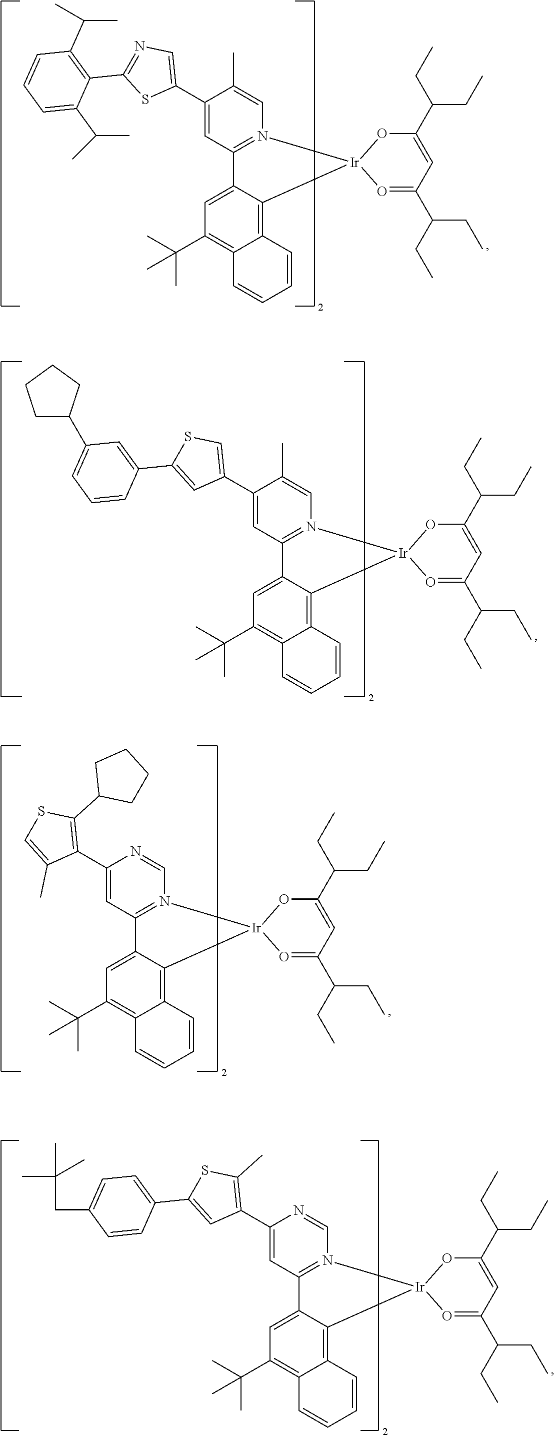

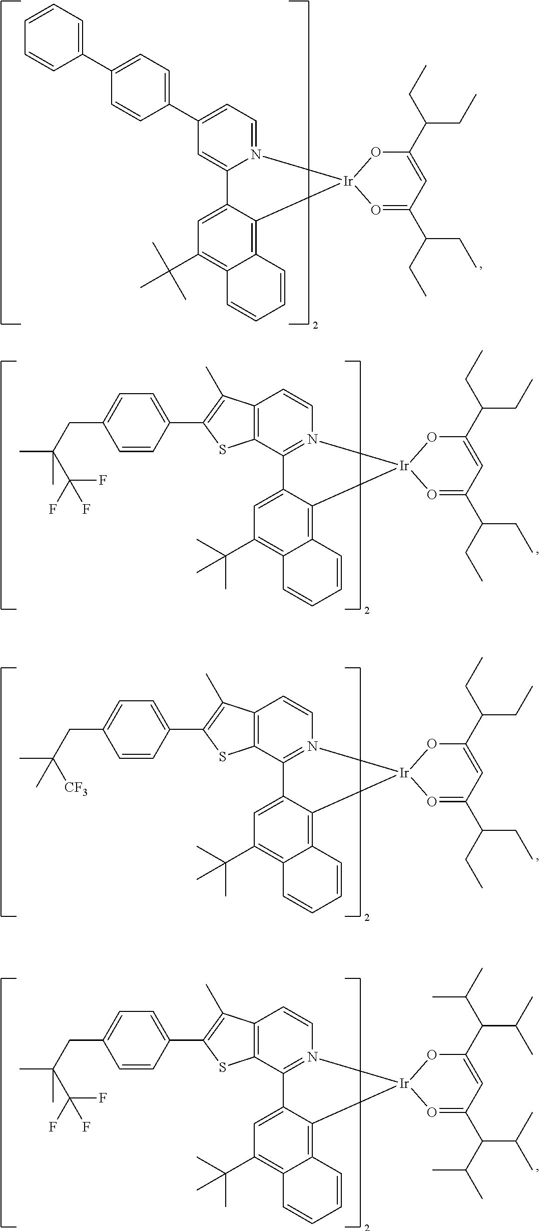

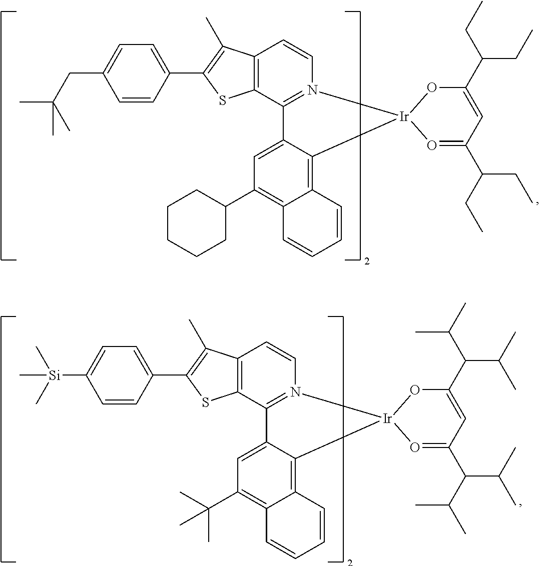

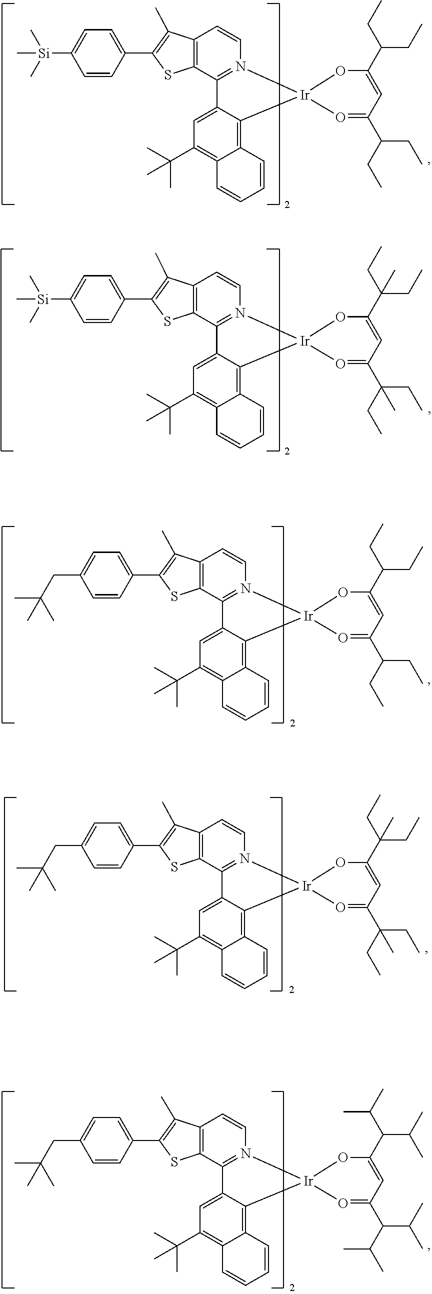

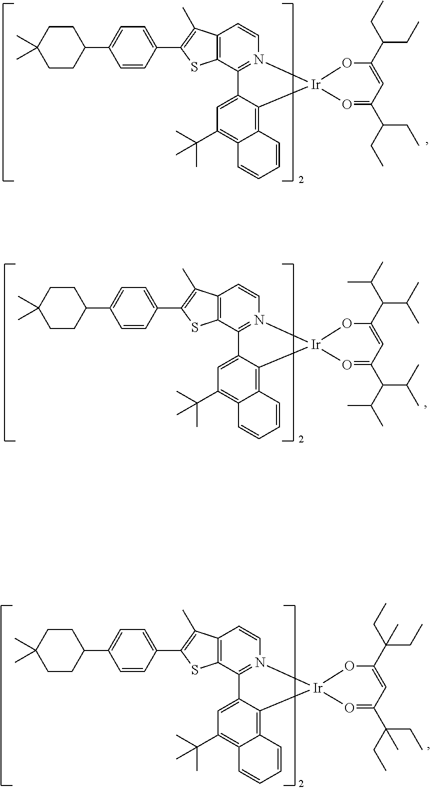

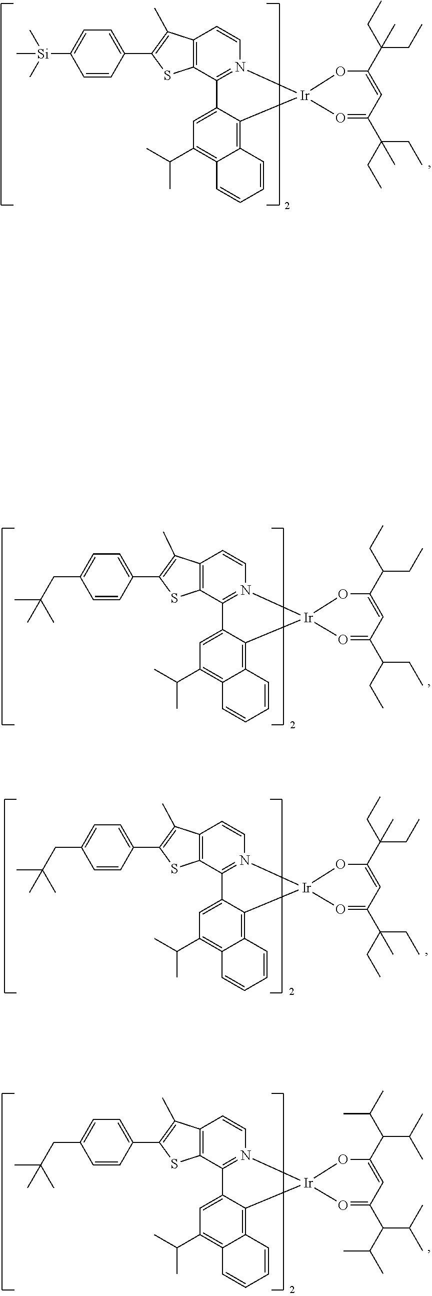

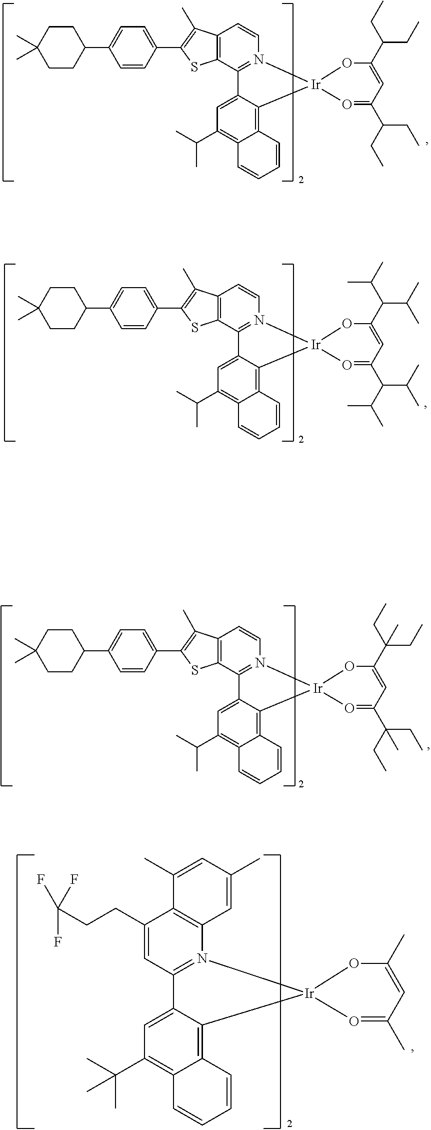

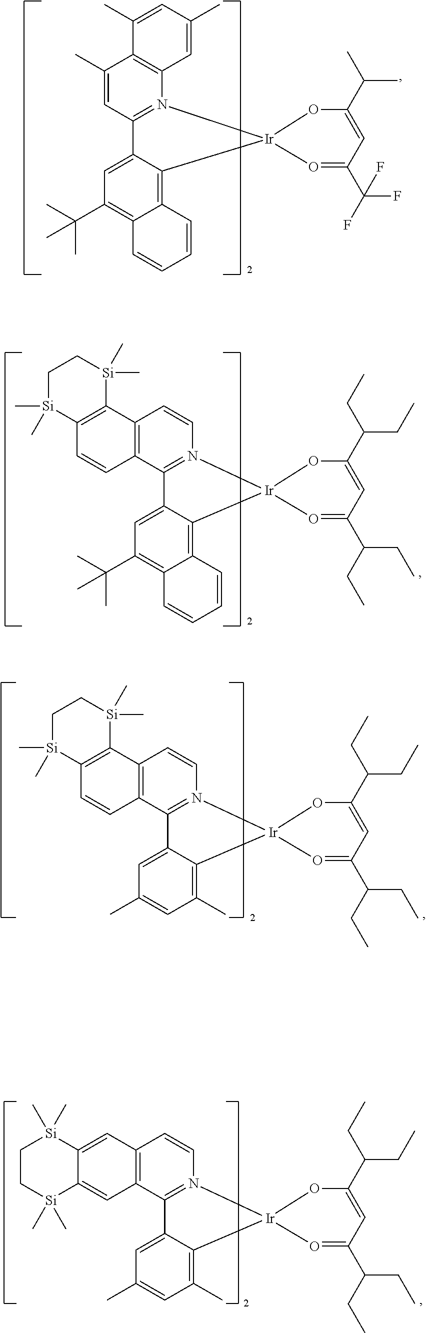

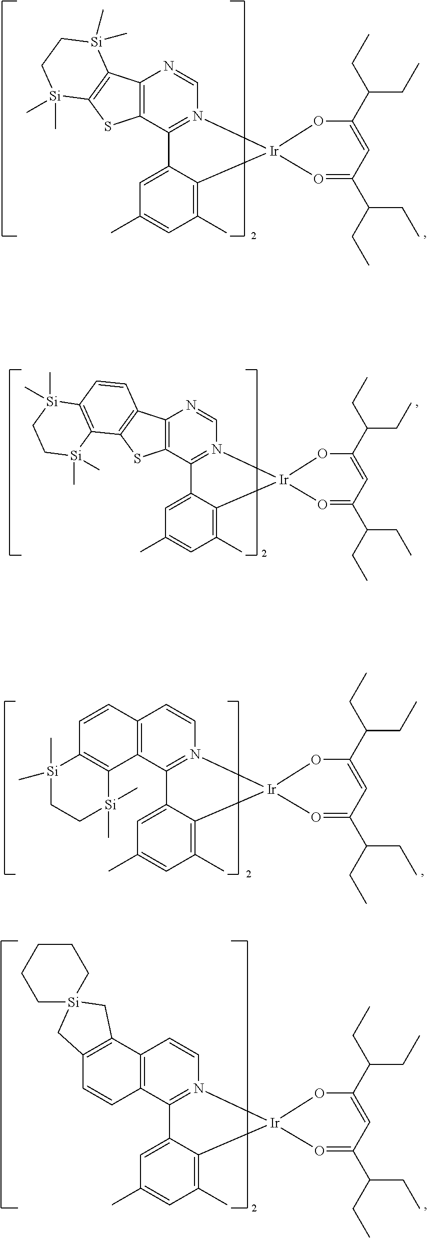

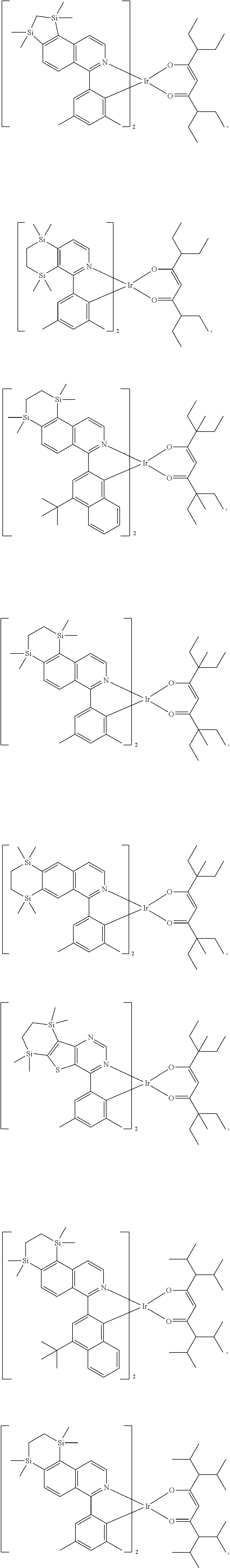

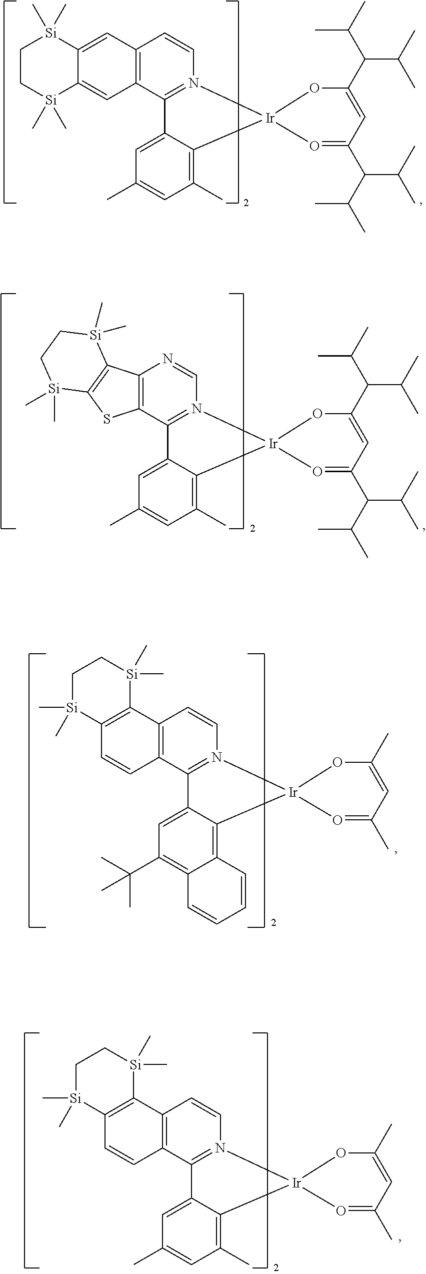

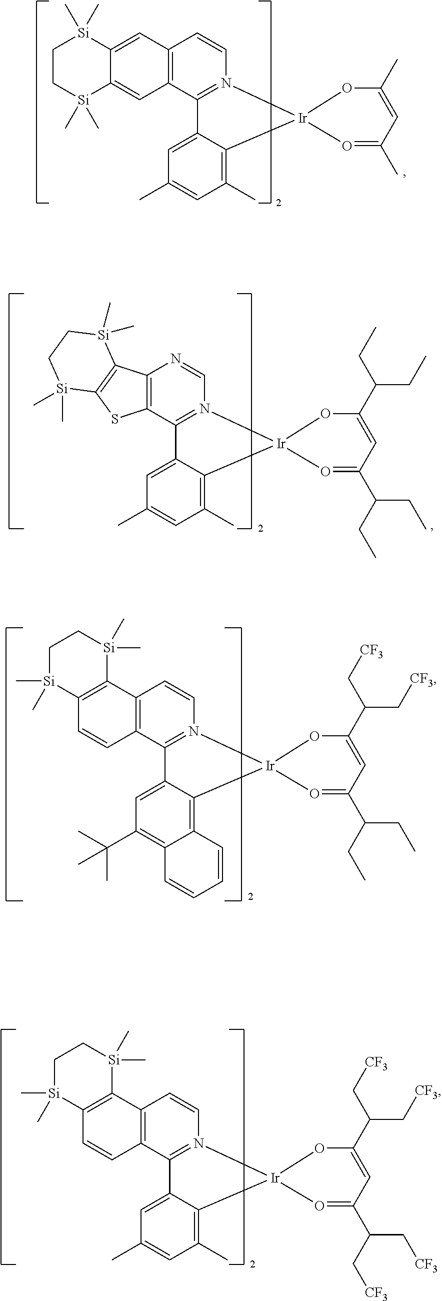

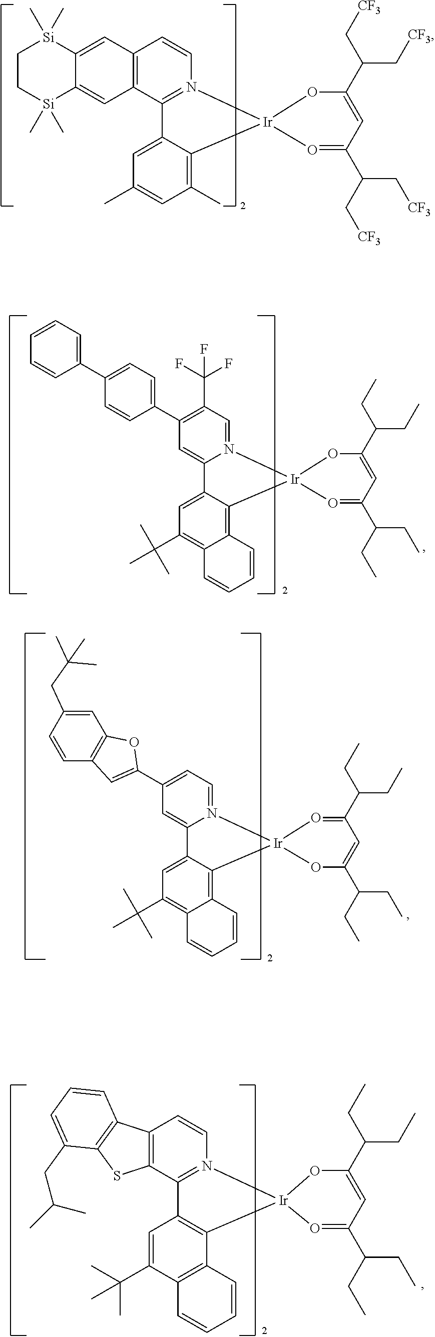

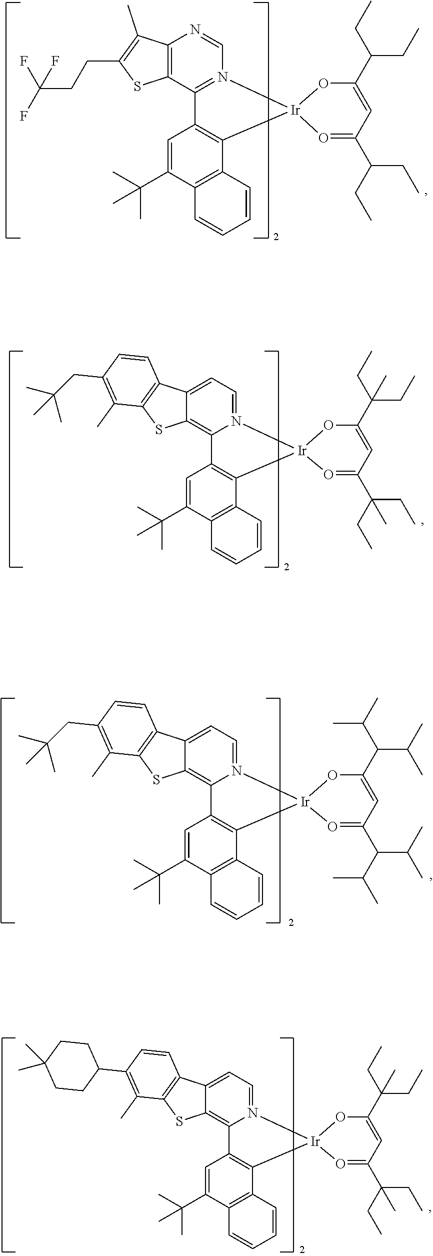

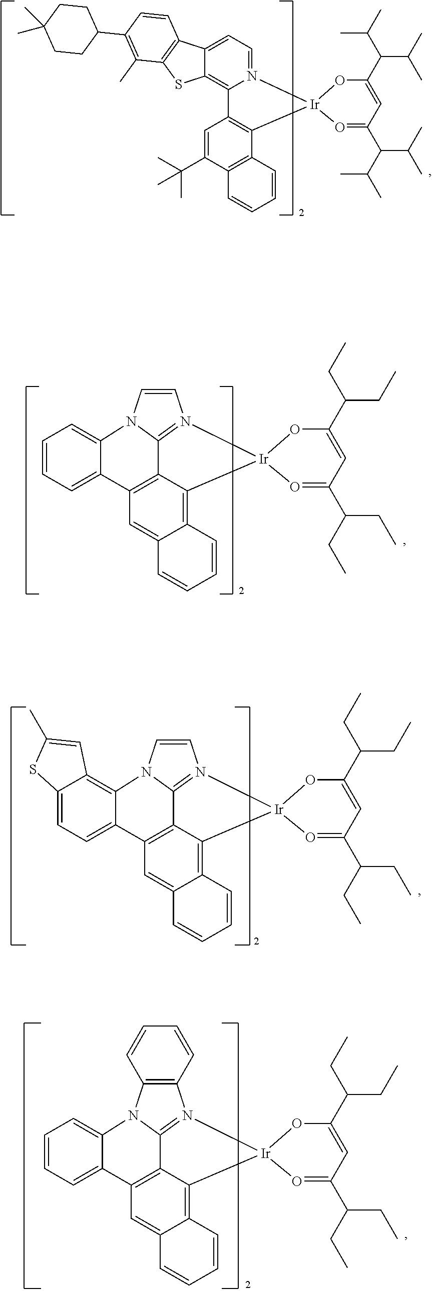

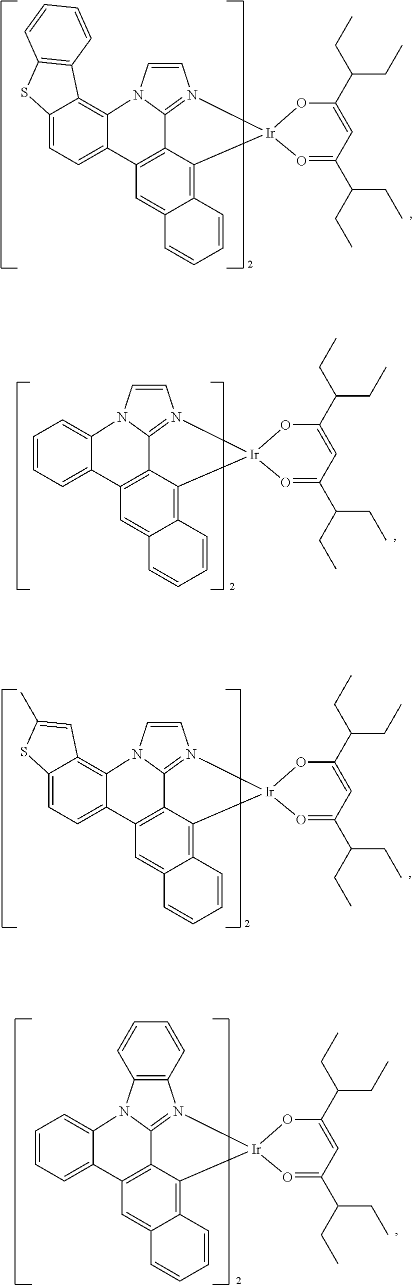

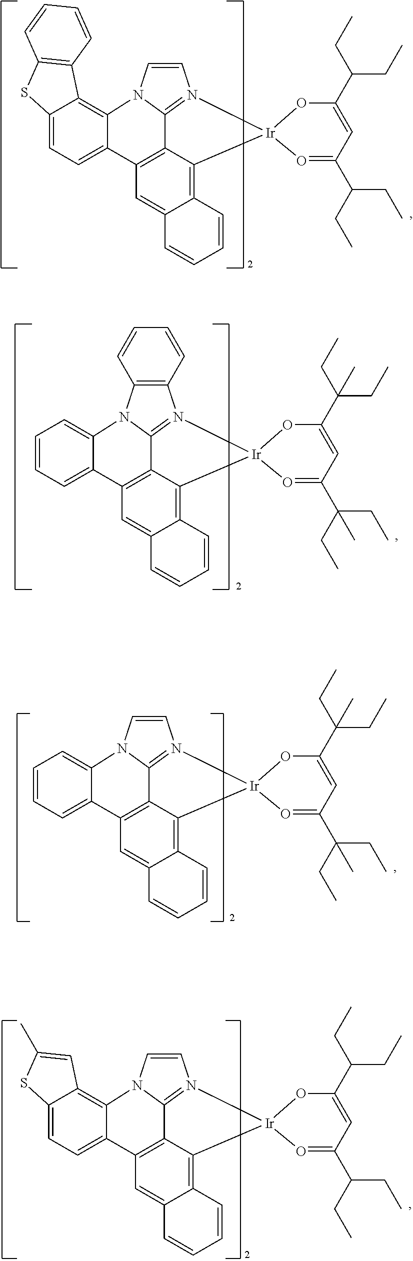

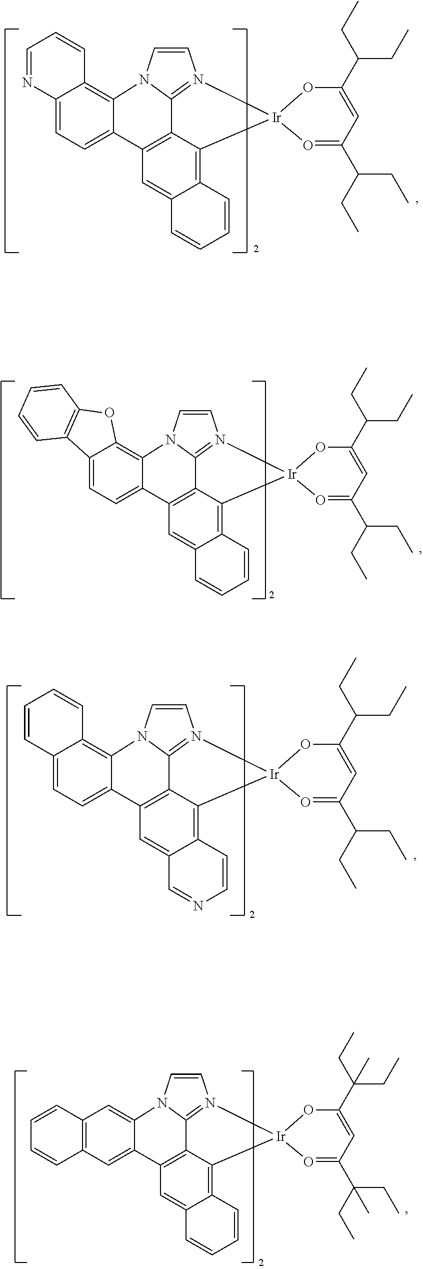

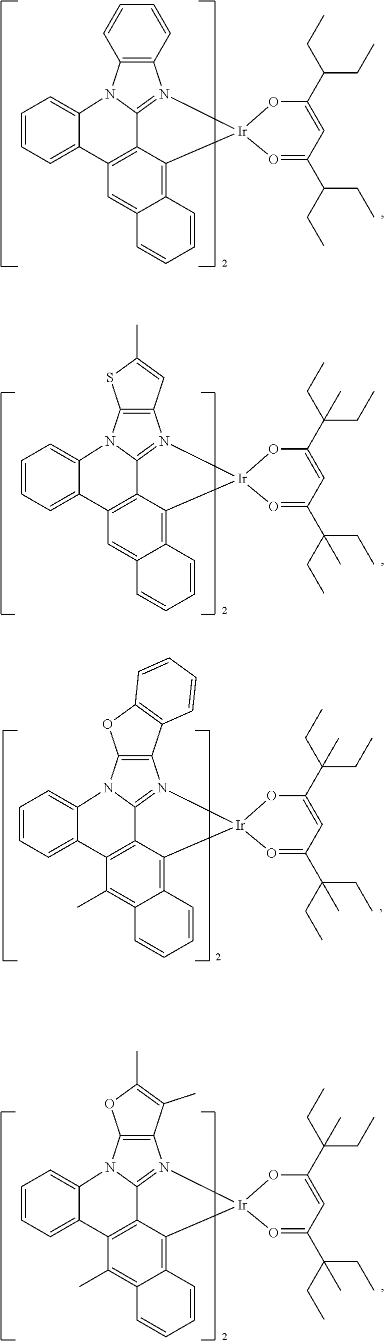

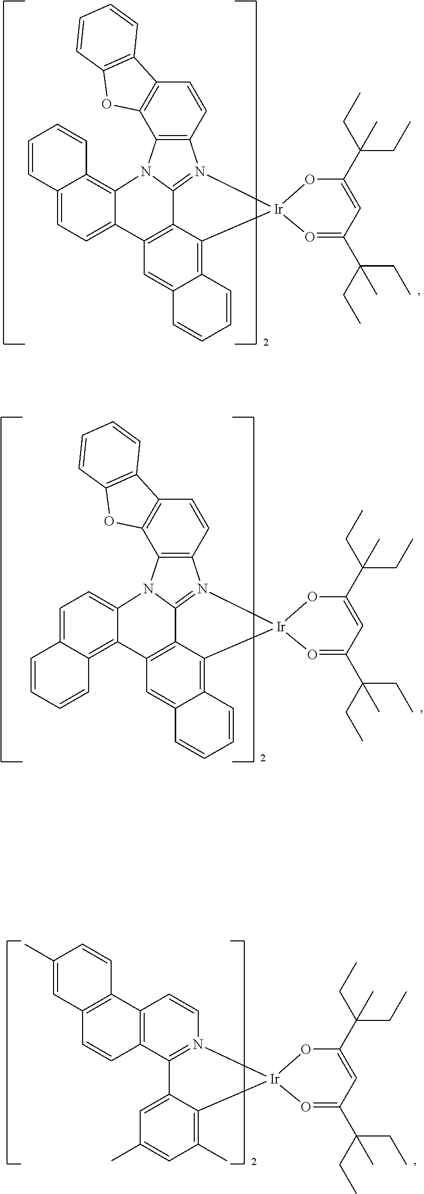

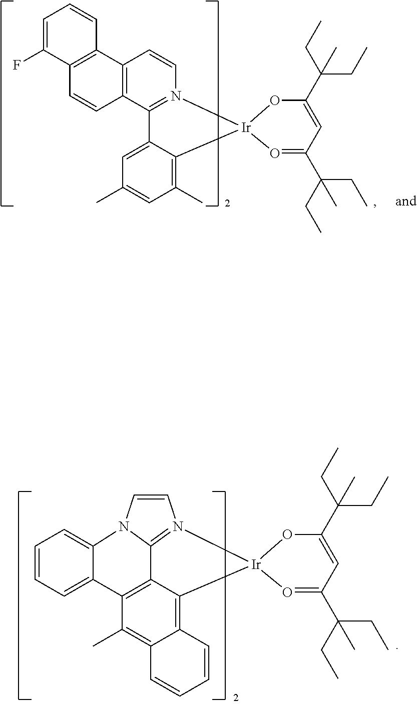

19. A consumer product comprising an OLED according to claim 1.

20. A premixed co-evaporation source that is a mixture that at least comprises a first compound and a second compound;
wherein the co-evaporation source is a co-evaporation source for vacuum deposition process or organic vapor jet printing process;
wherein the first compound and the second compound are different;
wherein the first compound, D1, is a first metal complex;
wherein the second compound, D2, is a second metal complex;
wherein the first compound has an evaporation temperature T1 of 150 to 400° C.;
wherein the second compound has an evaporation temperature T2 of 150 to 400° C.;
wherein an absolute value of T1−T2 is less than 20° C.;
wherein the first compound has a concentration C1 in said mixture and a concentration C2 in a film formed by evaporating the mixture in a vacuum deposition tool at a constant pressure between 1×10⁻⁶ Torr to 1×10⁻⁹ Torr, at a 2 Å/sec deposition rate on a surface positioned at a predefined distance away from the mixture being evaporated; and
wherein an absolute value of (C1−C2)/C1 is less than 5%.
